(19) United States
(12) Patent Application Publication
(10) Pub. No.: US 2022/0153741 A1
(43) Pub. Date: May 19, 2022

(54) HETEROCYCLIC COMPOUNDS AS INHIBITORS OF KRAS G12C

(71) Applicant: Guangdong Newopp Biopharmaceuticals Co., Ltd., Guangzhou (CN)

(72) Inventors: Zhaoyin Wang, Guangzhou (CN); Nanxin Li, Guangzhou (CN)

(73) Assignee: Guangdong Newopp Biopharmaceuticals Co., Ltd., Guangzhou (CN)

(21) Appl. No.: 17/556,086

(22) Filed: Dec. 20, 2021

Related U.S. Application Data
(63) Continuation-in-part of application No. PCT/CN2020/097802, filed on Jun. 23, 2020.
(60) Provisional application No. 62/995,304, filed on Jan. 23, 2020.
(60) Provisional application No. 62/921,513, filed on Jun. 24, 2019.

(51) Int. Cl. C07D 471/22; C07D 498/22; C07D 513/22; C07D 471/18; C07D 498/18; C07D 513/18; C07D 515/22

(52) U.S. Cl. CPC C07D 471/22; C07D 498/22; C07D 513/22; C07D 515/22; C07D 498/18; C07D 513/18; C07D 471/18

(57) ABSTRACT

Heterocyclic compounds or pharmaceutically acceptable salt thereof are provided as inhibitors of the KRAS G12C mutant, and compositions containing these compounds which may be used to treat various disease conditions associated with KRAS G12C, such as cancers.

### RELATED APPLICATIONS

This application claims priority benefit of, and is a continuation of part of International Application Serial PCT/CN2020/097802 filed 23 Jun. 2020; that in turn claims priority benefit of U.S. Provisional Application Ser. No. 62/921,513 filed 24 Jun. 2019; U.S. Provisional Application Ser. No. 62/995,304 filed 23 Jan. 2020; the contents of the aforementioned priority applications are all hereby incorporated by reference.

### BACKGROUND

RAS proteins are small, membrane-bound guanine nucleotide-binding proteins; they act as molecular switches by cycling between active GTP-bound and inactive GDP-bound conformations. There are 3 major isoforms of RAS proteins: KRAS, NRAS and HRAS. They play a crucial role in the regulation of cell proliferation, differentiation and survival. Hyperactivating mutations of major RAS isoforms are among the most common lesions found in cancer, with KRAS mutations being by far the most common in human cancer. Most of these mutations have been shown to cause an increase in the active GTP-bound population, leading to oncogenic transformation. The most frequent site of oncogenic mutation in KRAS is residue G12, with G12C mutation (glycine-12 to cysteine) as one of the frequent mutations at this residue. KRAS G12C mutation is found in ˜14% of lung adenocarcinoma and 1%-4% of pancreatic and colorectal adenocarcinomas, respectively. Given the role and the frequency of KRAS G12C mutation in human cancers, there is a strong need for new medical treatments for patients with cancers characterized by KRAS G12C mutation.

### SUMMARY OF THE INVENTION

The present invention describes inhibitors of KRAS G12C. The present invention further describes pharmaceutical formulations that include an inhibitor of KRAS G12C.

In one aspect, the invention features a compound of Formula I, or a pharmaceutically acceptable salt thereof:

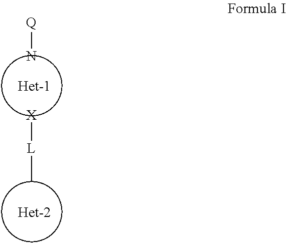

Wherein

Q is a moiety capable of forming a covalent bond with a nucleophile and preferably structures of exemplary Q are shown below:

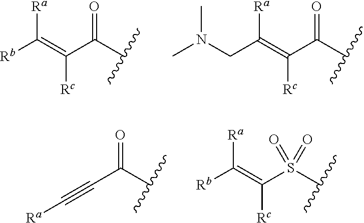

X is N, C, CR³, or CF; R³ is H, —CN, or C₁₋₆alkyl;

-L- is a single bond, a double bond, —NH— or —N(C₁₋₆alkyl)-;

Each R^{a}, R^{b}, and R^{c} is, independently, H, halogen, substituted or unsubstituted C₁₋₄alkyl, substituted or unsubstituted C₁₋₄cycloalkyl, or cyano; or R_{c} can be connected with a carbon atom of Het-1 to form a bicyclic ring.

Het-1 is selected from the following bicyclic and tricyclic moieties:

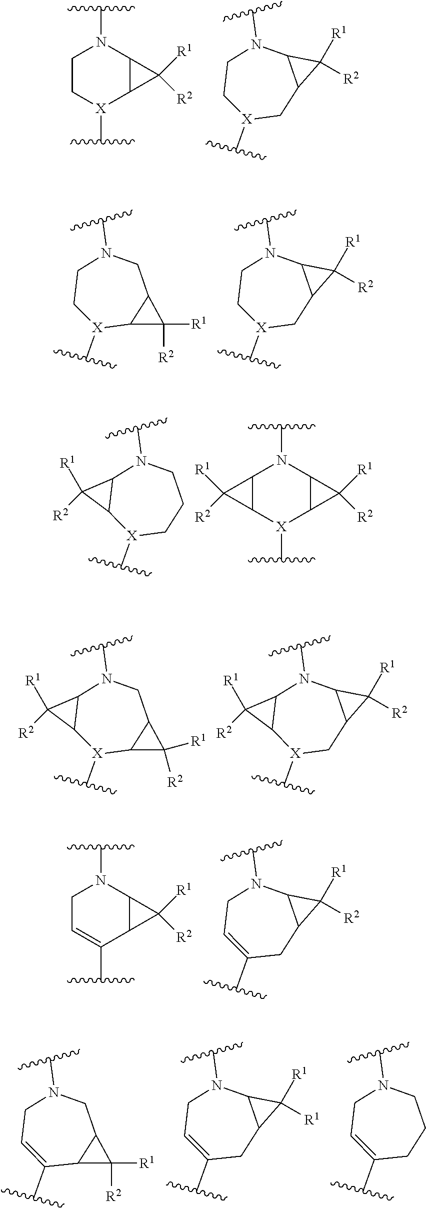

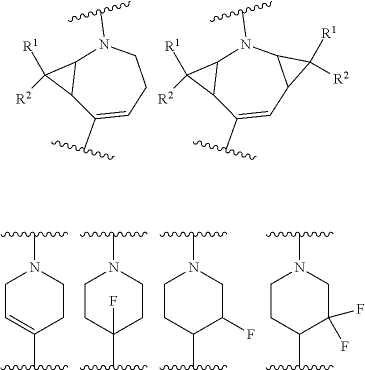

can also be selected from the following moieties:

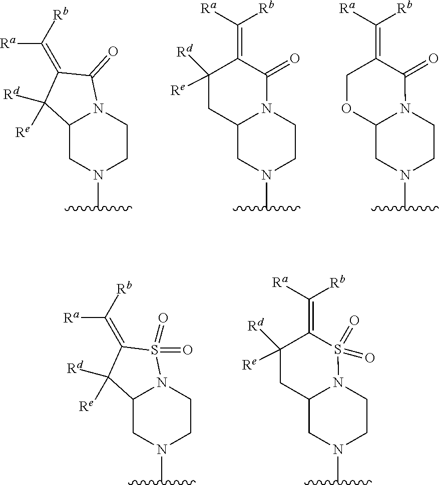

R^{e} and R^{d} are independently selected from hydrogen, halo;

R¹ and R² are independently selected from hydrogen, halo, cyano, C₁₋₆ alkoxy, hydroxy, C(O)NH₂, C(O)NHC₁₋₆alkyl, C(O)N(C₁₋₆alkyl)₂, C₁₋₆alkylsulfonyl, S(O)₂NH₂, S(O)₂NHC₁₋₆alkyl, NHC(O)NH₂, NHC(O)NHC₁₋₆alkyl, C₁₋₆alkyl, NHC(O)OC₁₋₆alkyl, C(O)—C₁₋₆alkyl, —C(O)C₁₋₆alkyl, C₁₋₆heteroalkyl, heterocyclyl, or heterocyclylalkyl; or R¹ and R², together with the carbon atom to which they are attached, can form a 3 to 6 membered carbocyclic ring.

Het-2 is selected from the following heterocyclic moieties:

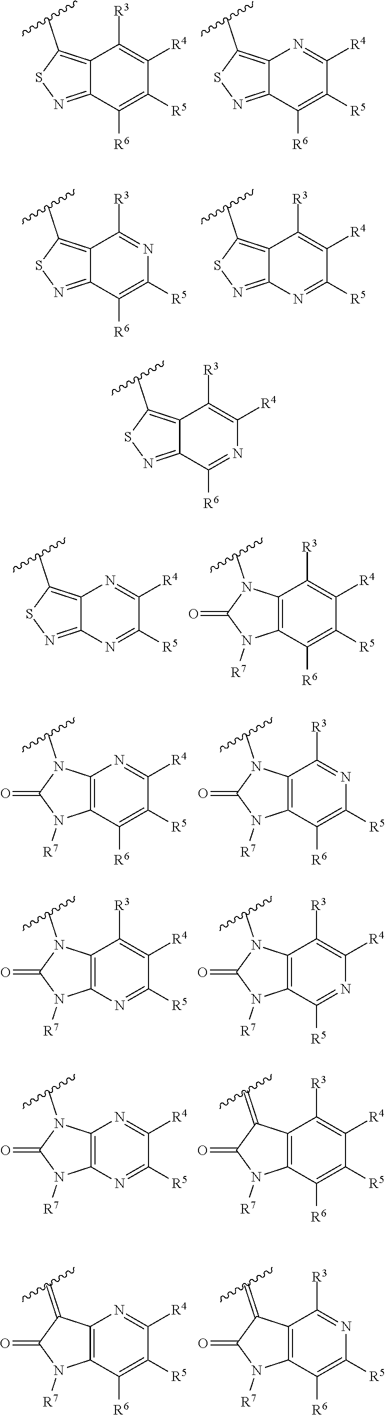

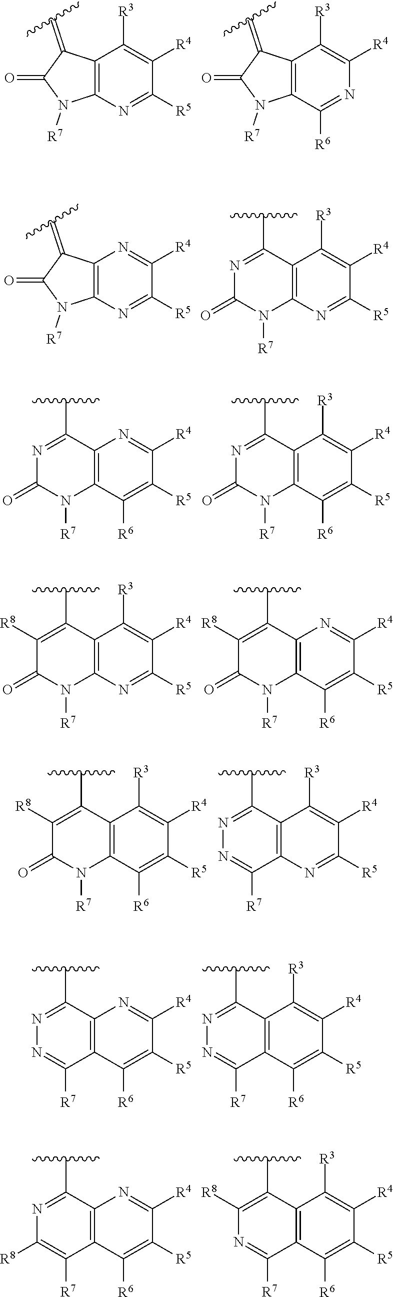

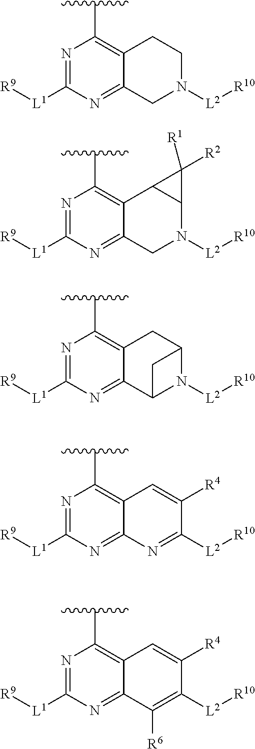

R³ and R⁶ are each independently H, OH, C₁₋₆alkyl, C₃₋₁₀cycloalkyl, C₃₋₁₀heterocylcoalkyl, C₁₋₆haloalkyl, C₁₋₆alkoxy, NH—C₁₋₆alkyl, N(C₁₋₆alkyl)₂, CN or halo; R⁴ is hydrogen, halo, C₁₋₆alkyl. C₁₋₆haloalkyl, C₁₋₆alkoxy, C₃₋₈cycloalkyl, C₂₋₄alkenyl, C₂₋₄alkynyl, aryl or heteroaryl;

R⁵ is halo, C₁₋₆alkyl. C₁₋₆haloalkyl, C₁₋₆alkoxy, OH, OR′, N(R′)₂, C₂₋₄alkenyl, C₂₋₄alkynyl, C₀₋₃alkylene-C₃₋₈cycloalkyl, C₀₋₃alkylene-C₃ halocycloalkyl, aryl or heteroaryl, C₀₋₃alkylene-C₆₋₄aryl or C₀₋₃alkylene-C₂₋₄heteroaryl, and each R′ is independently H, C₁₋₆alkyl. C₁₋₆haloalkyl, C₃₋₈cycloalkyl, C₂₋₄alkenyl, C₂₋₄alkynyl, aryl or heteroaryl; or two R′ substituents, together with the nitrogen atom to which they are attached, form a 3-8-membered carbocycle or 3-8-membered heterocycle containing O, S or NR′;

R⁷ is C₁₋₈alkyl, C₀₋₃alkylene-C₆₋₁₄aryl, C₀₋₃alkylene-C₂₋₁₄heteroaryl, C₀₋₃alkylene-C₃₋₁₀cycloalkyl, C₀₋₃alkylene-C₂₋₁₀heterocycloalkyl, C₁₋₆alkoxy, O—C₀₋₃alkylene-C₆₋₁₄aryl, O—C₀₋₃alkylene-C₃₋₁₄heteroaryl, O—C₀₋₃alkylene-C₁₋₁₀cycloalkyl, O—C₀₋₃alkylene-C₂₋₁₄heterocycloalkyl, NH—C₁₋₈alkyl, N(C₁₋₈alkyl)₂, NH—C₀₋₃alkylene-C₆₋₁₄aryl, NH—C₀₋₃alkylene-C₂₋₁₄heteroaryl, NH—C₀₋₃alkylene-C₁₋₁₀cycloalkyl, NH—C₀₋₃alkylene-C₂₋₁₄heterocycloalkyl, halo, —CN or C₁₋₆alkylene-amine;

R⁸ is H, OH, C₁₋₆alkyl, C₁₋₆cycloalkyl, C₁₋₆haloalkyl, C₁₋₆halocycloalkyl, C₁₋₆alkoxy, NH—C₁₋₆alkyl, N(C₁₋₆alkyl)₂, or CN;

L¹ is a bond, O, S or NR¹¹;

L² is a bond, —C(O)—, or C₁₋₃alkylene;

R⁹ is hydrogen, C₁₋₈alkyl, hydroxyC₁₋₈alkyl, dihydroxyC₁₋₈alkyl, C₁₋₈alkyl-NH—C₁₋₈alkyl, C₁₋₈alkyl-N(C₁₋₈alkyl)₂, —C₁₋₄alkylene-NR¹¹R¹², C₂₋₁₀heterocyclyl, C₂₋₁₀heterocyclylalkyl, C₆₋₁₄aryl, C₂₋₁₄heteroaryl, or C₃₋₁₄heteroarylalkyl, wherein R⁹ may be optionally substituted with one or more R³;

R⁹-L¹- can also be absent;

R¹⁰ is hydrogen, C₃₋₁₀cycloalkyl, C₃₋₁₀heterocyclyl, C₆₋₁₄aryl, C₇₋₂₀aralkyl or C₃₋₁₄heteroaryl, wherein each of the C₃₋₁₀cycloalkyl, C₃₋₁₀heterocyclyl, C₆₋₁₄aryl, C₇₋₂₀aralkyl or C₃₋₁₄heteroaryl may be optionally substituted with one or more R⁵ or R⁶;

optionally R³, R⁴, R⁵, R⁶, R⁷, R⁵, R⁹, and R¹⁰ are each independently substituted with one or more: halo, cyano, C₁₋₆ alkoxy, hydroxy, amino, C(O)NH₂, C(O)NHC₁₋₆alkyl, C(O)NHC₃₋₆cycloalkyl, C(O)N(C₁₋₆alkyl)₂, SC₁₋₆alkyl, S(O)C₁₋₆alkyl, S(O)₂C₁₋₆alkyl, SC₃₋₆cycloalkyl, S(O)C₃₋₆cycloalkyl, S(O)₂C₁₋₆cycloalkyl, S(O)₂NH₂, S(O)₂NHC₁₋₆alkyl, S(O)₂NHC₃₋₆alkyl, NHC(O)NH₂, NHC(O)NHC₁₋₆alkyl, NHC(O)NHC₁₋₆cycloalkyl, C₁₋₆alkyl, C₃₋₆cycloalkyl, NHC(O)OC₁₋₆alkyl, C(O)—C₁₋₆cycloalkyl, C(O)C₁₋₆alkylamino, C₁₋₆heteroalkyl, P(O)(C₁₋₆alkyl)₂, heterocyclyl, or heterocyclylalkyl;

the substituents on R⁵ and R⁷ can be connected through a carbon-carbon bond, a carbon-carbon double bond, a carbon-nitrogen bond, an amide bond, an ether bond, an ester bond and a sulfide bond to form a macrocyclic ring;

R¹¹ is H or C₁₋₃alkyl; R¹² is independently hydrogen, acyl, C₁₋₈alkyl, C₁₋₈haloalkyl or C₁-8hydroxyalkyl;

R¹³ is independently hydrogen, oxo, acyl, hydroxyl, C₁₋₈hydroxyalkyl, cyano, halogen, C₁₋₈alkyl, aralkyl, C₁₋₈haloalkyl, C₁₋₈heteroalkyl, C₁₋₁₀cycloalkyl, C₁₋₁₀heterocyclylalkyl, C₁₋₈alkoxy, N(C₁₋₈alkyl)₂, C₁₋₈alkyl-N(C₁₋₈alkyl)₂, or —C₁₋₄alkylene-NR¹¹R¹², wherein the C₁₋₈alkyl may be optionally substituted with one or two substituents selected from R¹, or C₁₋₈cycloalkyl;

each alkyl, alkenyl, alkynyl, cycloalkyl, aryl, heteroaryl can be substituted with the substituents which are defined in the section of Definitions.

In another aspect, the invention provided a compound of Formula II, or a pharmaceutically acceptable salt thereof:

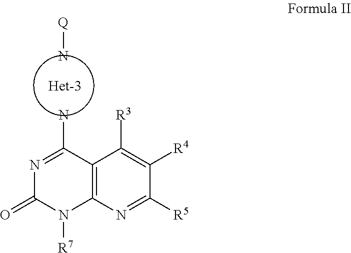

Wherein

R³, R⁴, R⁵ and R⁷ are defined as above;

Q is selected from the following moieties:

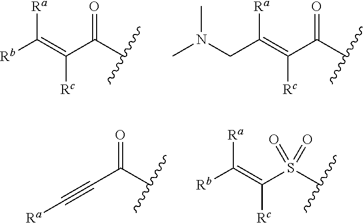

R^{a}, R^{b} and R^{c} are defined as above;

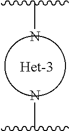

is selected from the following bicyclic and tricyclic moieties:

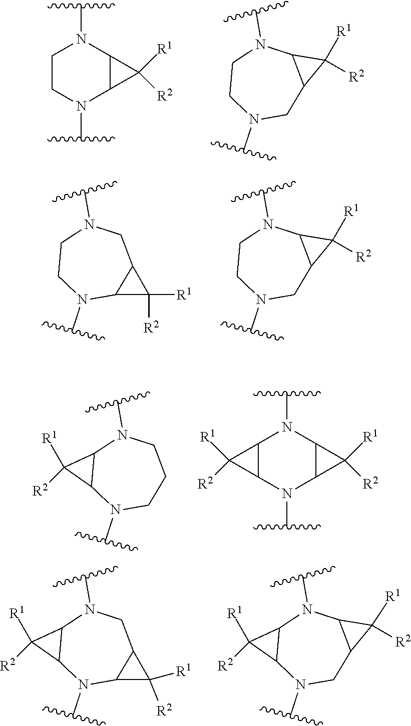

R¹ and R² are defined as above;

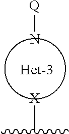

can also be selected from the following moieties:

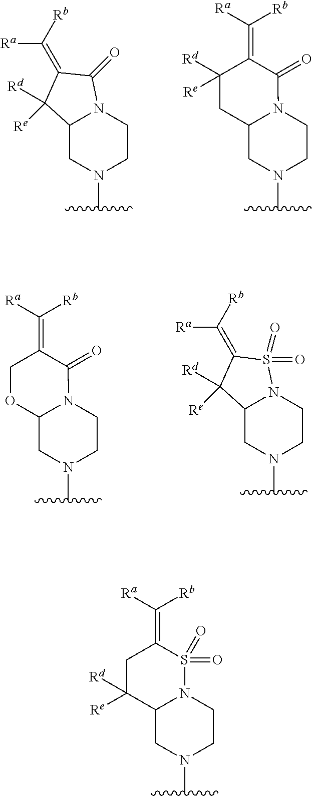

R^{e} and R^{d} are independently selected from hydrogen, halo;

R^{a} and R^{b} are defined as above.

In another aspect, the invention provided a compound of Formula III, or a pharmaceutically acceptable salt thereof:

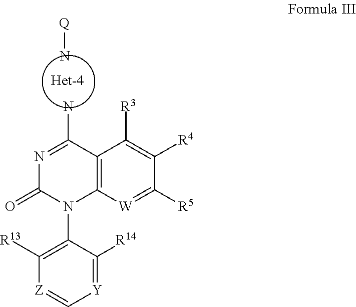

Wherein

R³, R⁴ and R⁵ and are defined as above;

Q is selected from the following moieties:

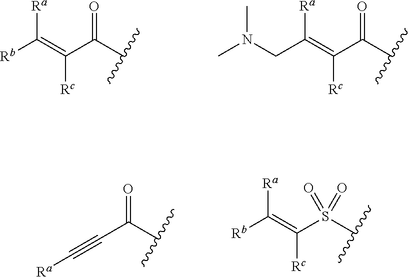

R^{a}, R^{b} and R^{c} are defined as above;

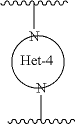

is selected from the following bicyclic and tricyclic moieties:

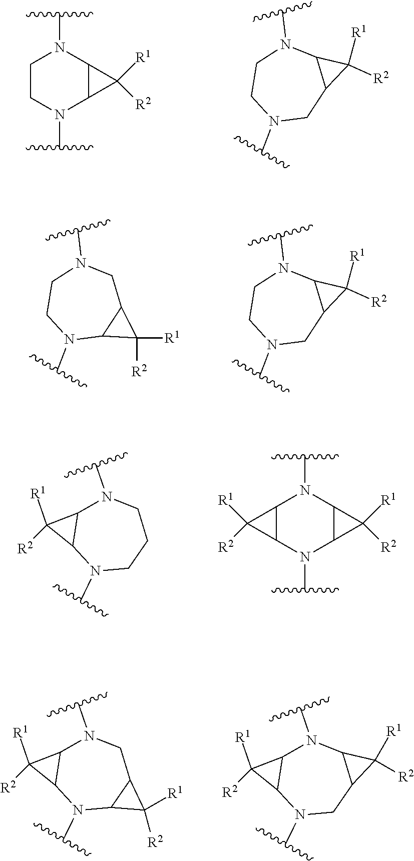

R¹ and R² are defined as above;

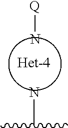

can also be selected from the following moieties:

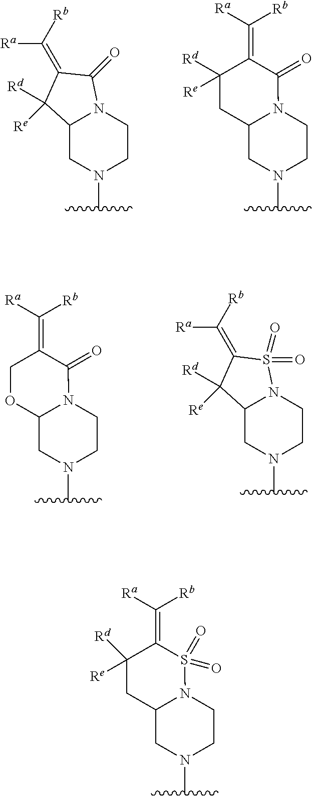

R^{e} and R^{d} are independently selected from hydrogen, halo;

R^{a} and R^{b} are defined as above;

Z and Y are each independently N or CR³;

W is N or CR⁶;

R⁶ is defined as above;

R¹³ and R¹⁴ are independently a branched or a linear C₁₋₆ alkyl, a branched or a linear C₁₋₆ alkenyl, C₃₋₆cycloalkyl, C₃₋₆ heterocycyl, —SC₁₋₆alkyl, —S(O)C₁₋₆alkyl, —S(O)₂C₁₋₆alkyl, —P(O)(C₁₋₆alkyl)₂, —OC₁₋₆alkyl. —OC₃₋₆ heterocycyl, —OC₃₋₆ cycyl, —SC₃₋₆ heterocycyl, —SC₃₋₆cycyl with a proviso that R¹³ and R¹⁴ are not a branched or a linear C₁₋₆ alkyl, C₃₋₆cycloalkyl at the same time.

In one aspect, the invention features a compound or Formula IV, or a pharmaceutically acceptable salt thereof:

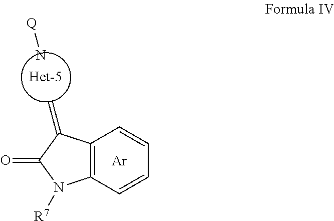

wherein

Q and R⁷ are defined as above;

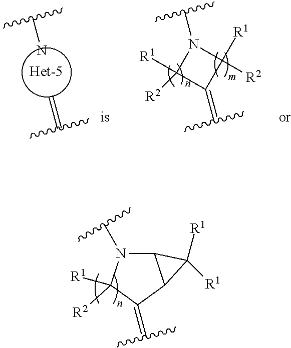

R¹ and R² are defined as above;

n and m are independently 0, 1, 2, 3, 4 and 5;

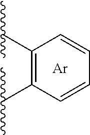

is selected from the following moieties:

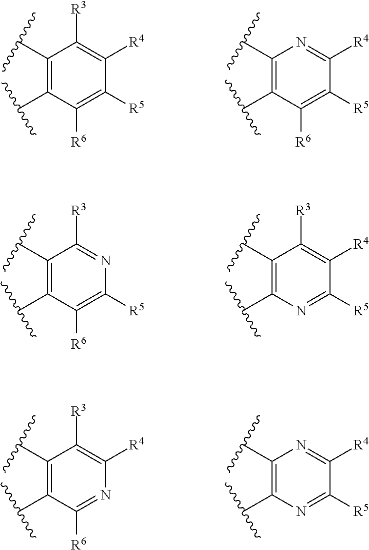

R³, R⁴, R⁵ and R⁶ are defined as above.

In one aspect, the invention provided a compound of Formula V, or a pharmaceutically acceptable salt thereof:

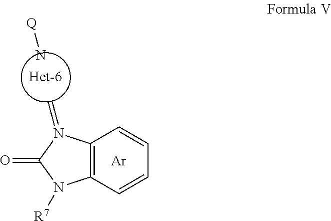

wherein

Q and R⁷ are defined as above;

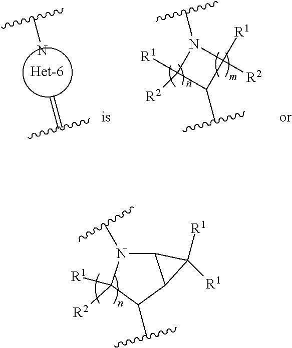

R¹ and R² are defined as above;

n and m are independently 0, 1, 2, 3, 4, or 5;

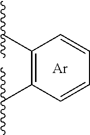

is selected from the following moieties:

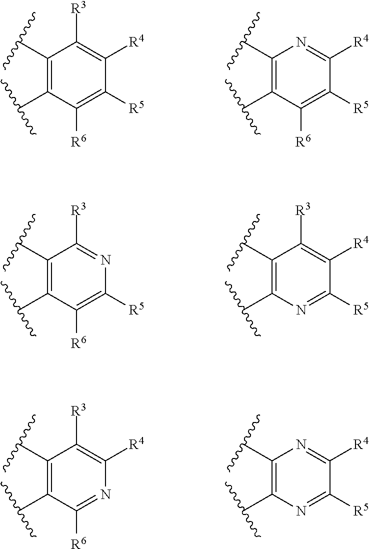

R³, R⁴, R⁵ and R⁶ are defined as above.

In one aspect, the invention provided a compound of Formula VI, or a pharmaceutically acceptable salt thereof:

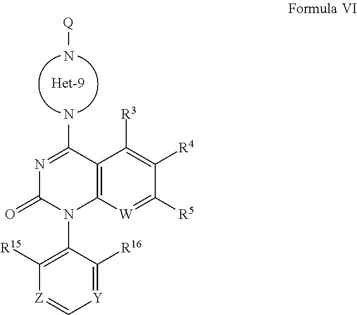

wherein

Q is defined as above;

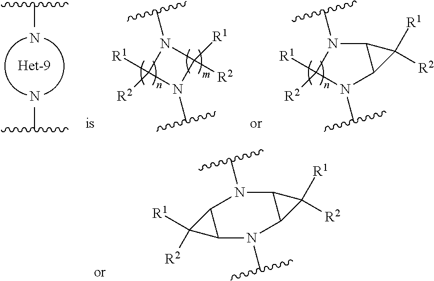

Z, Y, R¹, R², R³, R⁴ and R⁵ are defined as above;

W is N or CR⁶;

R⁶ is defined as above;

n and m are independently 0, 1, 2, 3, 4, or 5;

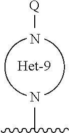

can also be selected from the following moieties:

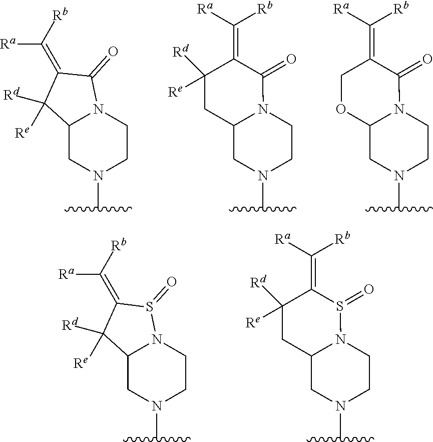

R^{e} and R^{d} are independently selected from hydrogen, halo;

R^{a} and R^{b} are defined as above;

R³, R⁴, and R⁵ are defined as above;

R¹⁵ is a branched or a linear C₁₋₆ alkyl, C₃₋₆cycloalkyl, C₃₋₆ heterocycyl, —SC₁₋₆alkyl, —OC₁₋₆alkyl, —OC₃₋₆ heterocycyl, —OC₃₋₆ cycyl, —SC₃₋₆ heterocycyl, or —SC₃₋₆cycyl;

R¹⁶ is —S(O)C₁₋₆alkyl, —S(O)₂C₁₋₆alkyl, —S(O)₂NHC₁₋₆alkyl, —S(O)₂N(C₁₋₆alkyl)₂, —P(O)(C₁₋₆alkyl)₂.

In one aspect, the invention provided a compound of Formula VII, or a pharmaceutically acceptable salt thereof:

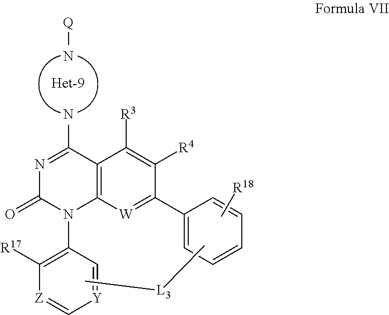

wherein

Q is defined as above;

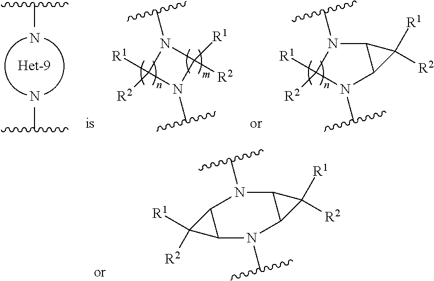

Z, Y, R¹, R², R³ and R⁴ are defined as above;

W is N or CR⁶;

R⁶ is defined as above;

n and m are independently 0, 1, 2, 3, 4, or 5;

R¹⁷ and R¹⁸ are independently selected from the group consisting of halogen, a branched or a linear C₁₋₆ alkyl, C₃₋₆cycloalkyl, C₃₋₆ heterocycyl, —SC₁₋₆alkyl, —OC₁₋₆alkyl. —OC₃₋₆ heterocycyl, —OC₃₋₆ cycyl, —SC₃₋₆ heterocycyl, —SC₃₋₆cycyl, —S(O)C₁₋₆alkyl, —S(O)₂C₁₋₆alkyl, —S(O)₂NHC₁₋₆alkyl, —S(O)₂N(C₁₋₆alkyl)₂, and —P(O)(C₁₋₆alkyl)₂;

L₃ is selected from the group consisting of —(CH₂)_{q}C(O)—, —O(CH₂)_{q}C(O)—, —NR¹⁹(CH₂)_{q}NR²⁰—, (CH₂)_{q}NR²⁰—, —O(CH₂)_{q}O—, —(CH₂)_{q}C(O)NR¹⁹—, —S(CH₂)_{q}C(O)—, —S(CH₂)_{q}C(O)—; —O(CH₂)_{q}C(O)NR¹⁹—, —O(CH₂)_{q}NR¹⁹—, —S(CH₂)_{q}O—, —O(CH₂)_{q}S—, —S(CH₂)_{q}S—, —NR¹⁹(CH₂)_{q}C(O)NR²⁰—, —NR¹⁹(CH₂)_{q}—, —NR¹⁹(CH₂)_{q}O—, —OC(O)(CH₂)_{q}—, —OC(O)(CH₂)_{q}—, —OC(O)(CH₂)_{q}S—, —(CH₂)_{q}CH═CH(CH₂)ᵣ—, —O(CH₂)_{q}CH═CH(CH₂)ᵣ—, —(CH₂)_{q}CH═CH(CH₂)ᵣO—, —O(CH₂)_{q}CH═CH(CH₂)ᵣO—, —S(CH₂)_{q}CH═CH(CH₂)ᵣ—, —(CH₂)_{q}CH═CH(CH₂)ᵣS—, —O(CH₂)_{q}CH═CH(CH₂)ᵣS—, —S(CH₂)_{q}CH═CH(CH₂)ᵣO—, —C(CH₂)_{q}S(CH₂)ᵣ—, and —C(CH₂)_{q}O(CH₂)ᵣ—;

q and r are independently selected from 1 to 6; preferably q and r are each independently 1, 2, 3, 4, 5 or 6;

R¹⁹ and R²⁰ are independently selected from hydrogen, C₁₋₆alkyl, C₃₋₆cycloalkyl;

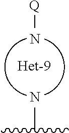

can also be selected from the following moieties:

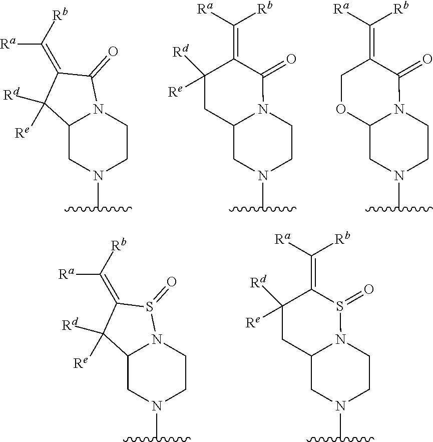

R^{e} and R^{d} are independently selected rom hydrogen, halo;

R^{a} and R^{b} are defined as above.

In one aspect, the invention provided a compound of Formula VIIA, or a pharmaceutically acceptable salt thereof:

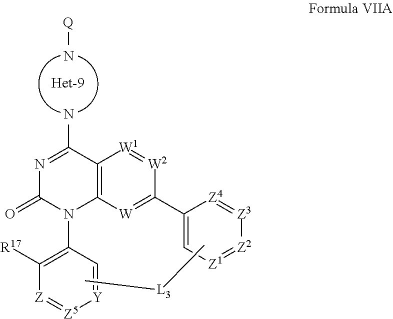

wherein

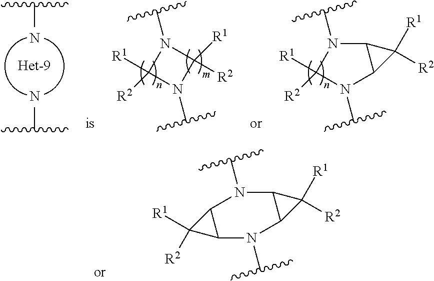

R¹ and R² are independently selected from hydrogen, halo, C₀₋₆alkylene-CN, C₀₋₆alkyleneNR¹⁹R²⁰, C₁₋₆alkoxy, hydroxy, C₀₋₆alkylene-C(O)NH₂, C₀₋₆alkylene-C(O)NHC₁₋₆alkyl, C₀₋₆alkylene-C(O)N(C₁₋₆alkyl)₂, C₀₋₆alkylene-S(O)₂—C₁₋₆alkyl, C₀₋₆alkylene-S(O)₂NH₂, C₀₋₆alkylene-S(O)₂NHC₁₋₆alkyl, C₀₋₆alkylene-S(O)₂N(C₁₋₆alkyl)₂, C₀₋₆alkylene-NHC(O)NH₂, C₀₋₆alkylene-NHC(O)NHC₁₋₆alkyl, C₀₋₆alkylene-NR¹⁹C(O)N(C₁₋₆alkyl)₂, C₁₋₆alkyl, C₀₋₆alkylene-NHC(O)OC₁₋₆alkyl, C₀₋₆alkylene-C(O)—C₁₋₆alkyl, C₁₋₆heteroalkyl, C₀₋₆alkylene-heterocyclyl, or C₀₋₆alkylene-heterocyclylalkyl; or R¹ and R², together with the carbon atom to which they are attached, can form a 3 to 6 membered carbocyclic ring;

Z and Y are independently N or CR³;

W is N or CR⁶;

W¹ is N or CR³;

W² is N or CR⁴;

Z¹, Z², Z³, Z⁴ and Z⁵ are independently N or CR¹⁸;

R³, R⁴ and R⁶ are independently H, OH, CN or halo, C₁₋₆alkyl, C₃₋₁₀cycloalkyl, C₃₋₁₀heteroalkyl, C₃₋₁₀heterocylcoalkyl, C₁₋₆haloalkyl, C₁₋₆alkoxy, NH—C₁₋₆alkyl, N(C₁₋₆alkyl)₂, C₃₋₈cycloalkyl, C₂₋₄alkenyl, C₂₋₄alkynyl, C₂₋₆heterocyclyl, aryl or heteroaryl;

R¹⁷ and R¹⁸ are independently selected from halogen, CN, a branched or a linear C₁₋₆ alkyl, C₃₋₆cycloalkyl, C₃₋₆ heterocycyl, —SC₁₋₆alkyl, —OC₁₋₆alkyl. —OC₃₋₆ heterocycyl, —OC₃₋₆ cycyl, NH—C₁₋₆alkyl, N(C₁₋₆alkyl)₂, —SC₃₋₆ heterocycyl, —SC₃₋₆cycyl, —S(O)C₁₋₆alkyl, —S(O)₂C₁₋₆alkyl, —S(O)₂NH₂, —S(O)₂NHC₁₋₆alkyl, —S(O)₂N(C₁₋₆alkyl)₂, —P(O)(C₁₋₆alkyl)₂, C₂₋₆heterocyclyl, an C₆₋₁₀aryl or a C₁₋₅heteroaryl;

L₃ is selected from —(CH₂)_{q}, —(CH₂)_{q}C(O)—, —O(CH₂)_{q}C(O)—, —NR¹⁹(CH₂)_{q}NR²⁰, —(CH₂)_{q}NR²⁰—, —O(CH₂)_{q}O—, —(CH₂)_{q}C(O)NR¹⁹—, —(CH₂)_{q}C(S)NR¹⁹—, —(CH₂)_{q}CHCF₃NR¹⁹—, —(CH₂)_{q}NR¹⁹C(O)—, —(CH₂)_{q}NR¹⁹CHCF₃—, —C(O)NR¹⁹(CH₂)_{q}—, —CHCF₃NR¹⁹(CH₂)_{q}—, —C(S)NR¹⁹(CH₂)_{q}—, —O(CH₂)_{q}C(S)NR¹⁹—, —S(O)ᵥ(CH₂)_{q}C(O)—, —O(CH₂)_{q}C(O)NR¹⁹—, —NR¹⁹C(O)(CH₂)_{q}C(O)NR²⁰—, —C(O)NR¹⁹(CH₂)_{q}C(O)NR²⁰—, —C(O)NR¹⁹(CH₂)_{q}NR²⁰C(O)—, —NR¹⁹C(O)(CH₂)_{q}NR²⁰C(O)—, O(CH₂)_{q})NR¹⁹—, —S(O)ᵥ(CH₂)_{q}O—, —O(CH₂)_{q}S(O)ᵥ—, —S(O)ᵥ(CH₂)_{q}—, —(CH₂)_{q}S(O)ᵥ—, —S(O)ᵥ(CH₂)_{q}S(O)ᵥ—, —NR¹⁹(CH₂)_{q}C(O)NR²⁰—, —NR¹⁹(CH₂)_{q}—, —NR¹⁹C(O)(CH₂)_{q}—, —NR¹⁹CHCF₃(CH₂)_{q}—, —NR¹⁹(CH₂)_{q}O—, —(CH₂)ᵣOC(O)(CH₂)_{q}—, —OC(O)(CH₂)_{q}—, —OC(O)(CH₂)_{q}S(O)ᵥ—, —(CH₂)_{q}CH═CH(CH₂)ᵣ—, —NR¹⁹(CH₂)_{q}CH═CH(CH₂)ᵣ—, NR¹⁹C(O)(CH₂)_{q}CH═CH(CH₂)ᵣ—, —(CH₂)_{q}CH═CH(CH₂)ᵣC(O)NR²⁰, —(CH₂)_{q}NR¹⁹C(O)NR²⁰(CH₂)ᵣ—, —(CH₂)_{q}NR¹⁹C(S)NR²⁰(CH₂)ᵣ—, —(CH₂)_{q}NR¹⁹S(O)₂NR²⁰(CH₂)ᵣ—, —(CH₂)_{q}S(O)ᵥ(CH₂)ᵣ—, —(CH₂)_{q}S(O)₂NR²⁰(CH₂)ᵣ—, —(CH₂)_{q}NR¹⁹S(O)ᵥ(CH₂)ᵣ—, —(CH₂)_{q}SS(CH₂)ᵣ—, —(CH₂)_{q}S(CH₂)ᵣ—, —(CH₂)_{q}O(CH₂)ᵣ—, —(CH₂)_{q}NR¹⁹(CH₂)ᵣ—, —(CH₂)_{q}C≡C(CH₂)ᵣ—, —O(CH₂)_{q}CH═CH(CH₂)ᵣ—, —O(CH₂)_{q}CH≡CH(CH₂)ᵣ—, —(CH₂)_{q}CH═CH(CH₂)ᵣO—, —(CH₂)_{q}CH≡CH(CH₂)ᵣO—, —O(CH₂)_{q}CH═CH(CH₂)ᵣO—, —O(CH₂)_{q}CH≡CH(CH₂)ᵣO—, —S(O)ᵥ(CH₂)_{q}CH═CH(CH₂)ᵣ—, S(O)ᵥ(CH₂)_{q}CH≡CH(CH₂)ᵣ—, —(CH₂)_{q}CH═CH(CH₂)ᵣS(O)ᵥ—, (CH₂)_{q}CH≡CH(CH₂)ᵣS(O)ᵥ—, —O(CH₂)_{q}CH═CH(CH₂)ᵣS(O)ᵥ—, —O(CH₂)_{q}CH≡CH(CH₂)ᵣS(O)ᵥ—, —S(O)ᵥ(CH₂)_{q}CH═CH(CH₂)ᵣO—, S(O)ᵥ(CH₂)_{q}CH≡CH(CH₂)ᵣO—, —C(CH₂)_{q}S(CH₂)ᵣ —, —C(CH₂)_{q}O(CH₂)ᵣ—, —C(O)NR¹⁹S(O)₂(CH₂)_{q}—, or —(CH₂)_{q}S(O)₂NR¹⁹C(O)—; or L₃ is L₄-L₅-L₆;

L₄ and L₆ are independently selected from —(CH₂)_{q}—, —O(CH₂)_{q}—, —S(CH₂)_{q}—, —NR¹⁹(CH₂)_{q}—, —(CH₂)_{q}NR²⁰—, —(CH₂)_{q}O—, —(CH₂)_{q}S—, —(CH₂)_{q}C(O)—, —C(O)(CH₂)_{q}—, —(CH₂)_{q}C(O)NR¹⁹, —NR¹⁹(C(O)(CH₂)_{q}—, —(CH₂)_{q}CH═CH(CH₂)ᵣ—, —O(CH₂)_{q}CH═CH(CH₂)ᵣ—, —(CH₂)_{q}CH═CH(CH₂)ᵣO—, —S(CH₂)_{q}CH═CH(CH₂)ᵣ—, —(CH₂)_{q}CH═CH(CH₂)ᵣS—, —O(CH₂)_{q}CH═CH(CH₂)ᵣS—, or —S(CH₂)_{q}CH═CH(CH₂)ᵣO—;

L⁵ is a C₂₋₆heterocyclyl, an C₆₋₁₀aryl or a C₁₋₉heteroaryl;

Each of the oxo group in L₃, L₄, L₅ and L₆ can be independently replaced with a thiocarbonyl group, —C(S)—, an oxetane group, or an imine group, —C(═NR¹⁹)—;

q and r are independently selected from 0 to 10; preferably q and r are each independently 0, 1, 2, 3, 4, 5, 6, 7, 8, 9, or 10;

v is 0, 1 or 2;

R¹⁹ and R²⁰ are independently selected from hydrogen, C₁₋₆alkyl, C₃₋₁₀heteroalkyl, C₃₋₆cycloalkyl, C₆₋₁₀aryl or a C₁₋₅heteroaryl, C₂₋₆heterocyclyl; or R¹⁹ and R²⁰ can be connected to form a ring;

Q is a moiety capable of forming a covalent bond with a nucleophile and preferably structures of exemplary Q are shown below:

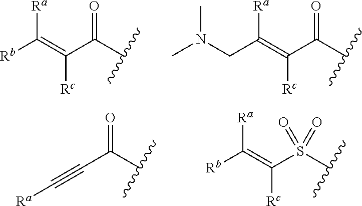

Each R^{a}, R^{b}, and R^{c} is, independently, H, halogen, substituted or unsubstituted C₁₋₄alkyl, substituted or unsubstituted C₁₋₄cycloalkyl, C₃₋₁₀heteroalkyl, or cyano.

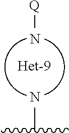

can also be selected from the following moieties:

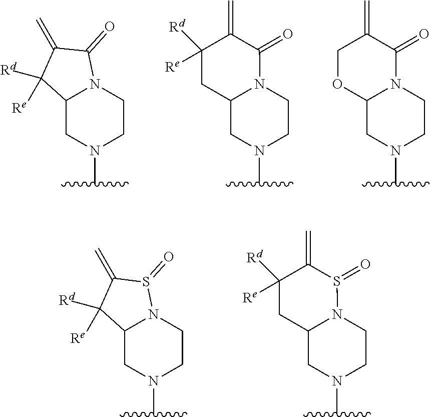

R^{e} and R^{d} are independently selected from hydrogen, halo, C₁₋₆alkyl, halogenated C₁₋₆alkyl, CN.

In another aspect, the invention provided a compound of Formula VIIB, or a pharmaceutically acceptable salt thereof:

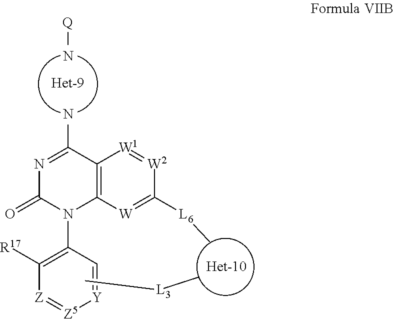

wherein

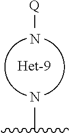

and Q are defined as above;

R¹⁷, Z, Z⁵, W, W¹, W², and L₃ are defined as above;

L₆ is selected from —(CH₂)_{q}—, —(CH₂)_{q}C(O)—, —O(CH₂)_{q}C(O)—, —NR¹⁹(CH₂)_{q}NR²⁰, —(CH₂)_{q}NR²⁰—, —O(CH₂)_{q}O—, —(CH₂)_{q}C(O)NR¹⁹—, —(CH₂)_{q}NR¹⁹C(O)—, —C(O)NR¹⁹(CH₂)_{q}—, —O(CH₂)_{q}C(O)NR¹⁹—, —S(O)ᵥ(CH₂)_{q}C(O)—, —NR¹⁹C(O)(CH₂)_{q}C(O)NR²⁰, —C(O)NR¹⁹(CH₂)_{q}C(O)NR²⁰—, —C(O)NR¹⁹(CH₂)_{q}NR²⁰C(O)—, —NR¹⁹C(O)(CH₂)_{q}NR²⁰C(O)—, O(CH₂)_{q}NR¹⁹—, —S(O)ᵥ(CH₂)_{q}O—, —O(CH₂)_{q}S(O)ᵥ—, —S(O)ᵥ(CH₂)_{q}S(O)ᵥ—, —NR¹⁹(CH₂)_{q}C(O)NR²⁰, —NR¹⁹(CH₂)_{q}—, —NR¹⁹C(O)(CH₂)_{q}—, —NR¹⁹(CH₂)_{q}O—, —O(CH₂)_{q}—, —(CH₂)_{q}CH═CH(CH₂)ᵣ—, —NR¹⁹(CH₂)_{q}CH═CH(CH₂)ᵣ—, NR¹⁹C(O)(CH₂)_{q}CH═CH(CH₂)ᵣ—, —(CH₂)_{q}CH═CH(CH₂)ᵣC(O)NR²⁰—, —(CH₂)_{q}C≡C(CH₂)ᵣ—, —O(CH₂)_{q}CH═CH(CH₂)ᵣ—, —O(CH₂)_{q}CH≡CH(CH₂)ᵣ—, —(CH₂)_{q}CH═CH(CH₂)ᵣO—, —(CH₂)_{q}CH≡CH(CH₂)ᵣO—, —O(CH₂)_{q}CH═CH(CH₂)ᵣO—, —O(CH₂)_{q}CH≡CH(CH₂)ᵣO—, —S(O)ᵥ(CH₂)_{q}CH═CH(CH₂)ᵣ—, S(O)ᵥ(CH₂)_{q}CH≡CH(CH₂)ᵣ—, —(CH₂)_{q}CH═CH(CH₂)ᵣS(O)ᵥ—, (CH₂)_{q}CH≡CH(CH₂)ᵣS(O)ᵥ—, —O(CH₂)_{q}CH═CH(CH₂)ᵣS(O)ᵥ—, —O(CH₂)_{q}CH≡CH(CH₂)ᵣS(O)ᵥ—, —S(O)ᵥ(CH₂)_{q}CH═CH(CH₂)ᵣO—, S(O)ᵥ(CH₂)_{q}CH≡CH(CH₂)ᵣO—, —C(CH₂)_{q}S(CH₂)ᵣ—, and —C(CH₂)_{q}O(CH₂)ᵣ—;

q and r are independently selected from 0 to 10; preferably q and r are each independently 0, 1, 2, 3, 4, 5, 6, 7, 8, 9, or 10;

v is 0, 1 or 2;

L₃ and L₆ may also be absent;

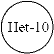

is C₆₋₁₀aryl, C₁₋₉heterocyclyl, C₁₋₉heteroaryl.

In another aspect, the invention features a compound of Formula VIIC, or a pharmaceutically acceptable salt thereof:

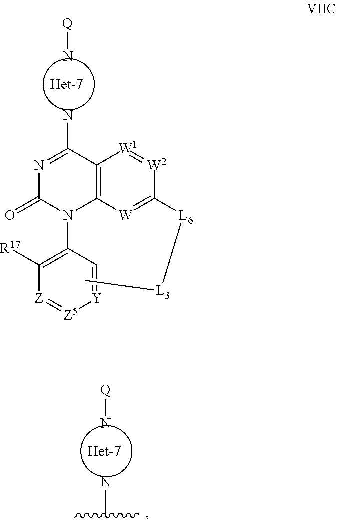

Y, R¹⁷, Z, Z⁵, W, W¹, W², L₃ and L₆ are defined as above;

In another aspect, the invention provided a compound of Formula VIII, or a pharmaceutically acceptable salt thereof:

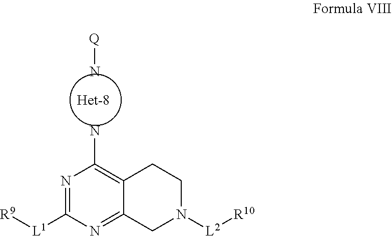

Wherein

Q, L¹, L², R⁹ and R¹⁰ are defined as above;

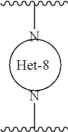

is selected from the following bicyclic and tricyclic moieties:

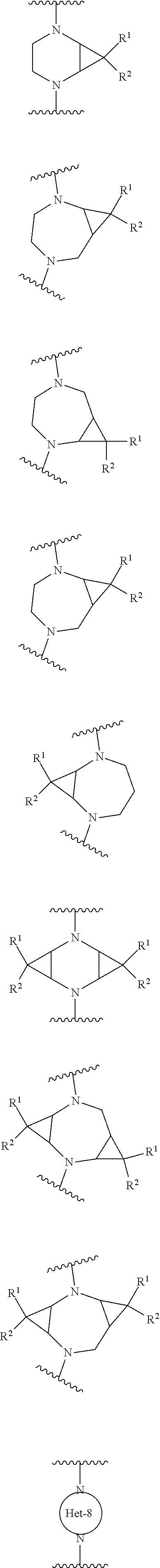

can also be selected the following moieties:

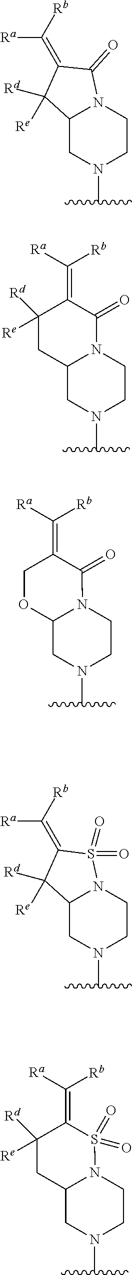

R^{a}, R^{b}, R^{e} and R^{d} are defined as above.

In another aspect, the invention provided a compound of Formula IX, or a pharmaceutically acceptable salt thereof:

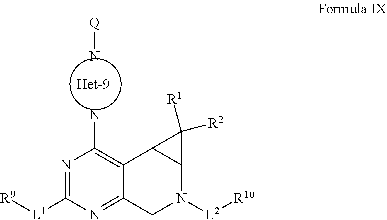

Wherein

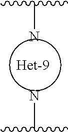

is selected from the following moieties:

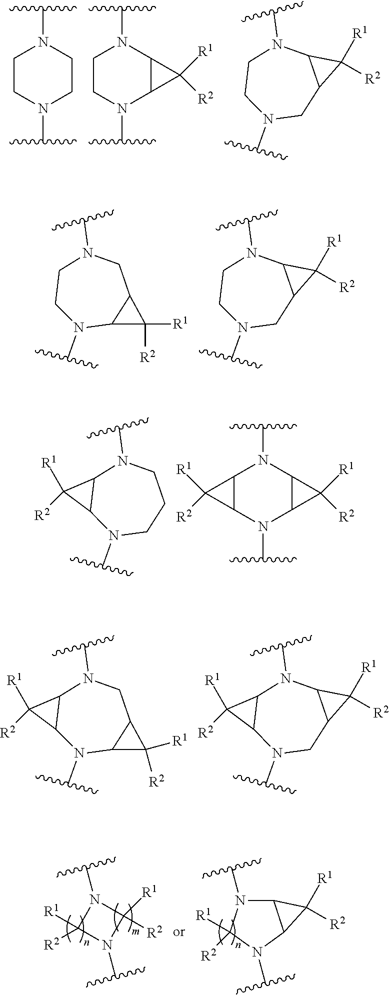

Q, n, m, L¹, L², R¹, R², R⁹ and R¹⁰ are defined as above;

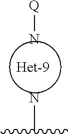

can also be selected from the following moieties:

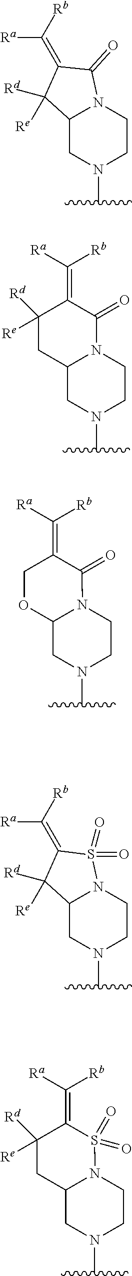

R^{a}, R^{b}, R^{e} and R^{d} are defined as above.

In another aspect, the invention provided a compound of Formula X, or a pharmaceutically acceptable salt thereof:

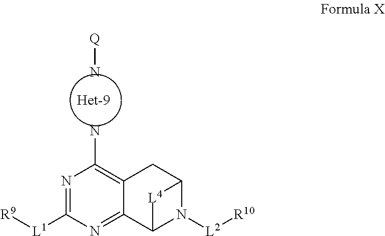

Wherein

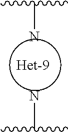

is selected from the following moieties:

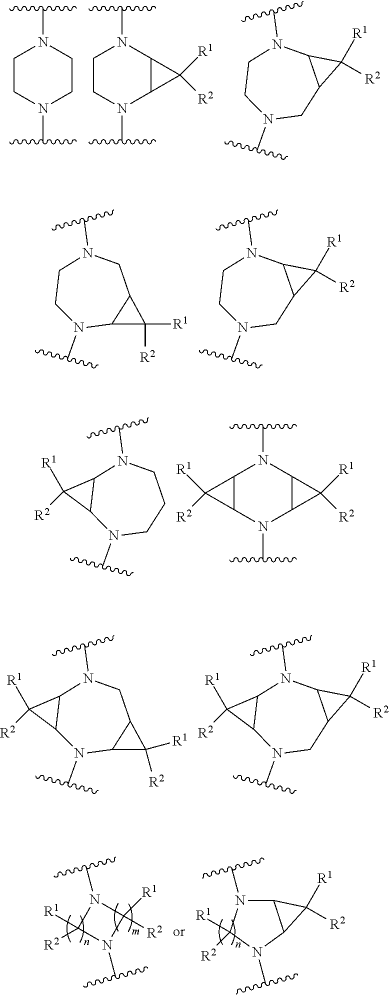

Q, n, m, L¹, L², R¹, R², R⁹ and R¹⁰ are defined as above;

L⁴ is —CR²¹R²²—, —(CR²¹R²²)₂—, O, S, NR²¹, NC(O)NR²¹, or NS(O)₂NR²¹R²².

R²¹ and R²² are independently selected from the group consisting of hydrogen, C₁₋₆alkyl, and C₃₋₆cycloalkyl;

or

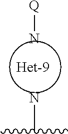

can also be selected from the following moieties:

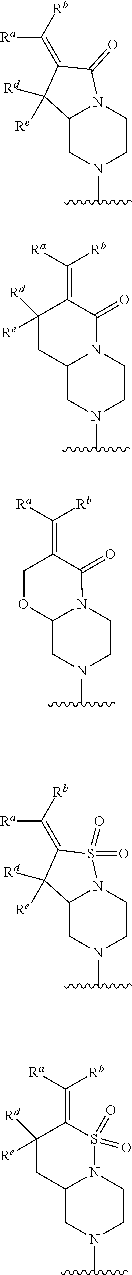

R^{a}, R^{b}, R^{e} and R^{d} are defined as above.

In another aspect, the invention provided a compound of Formula XI, or a pharmaceutically acceptable salt thereof:

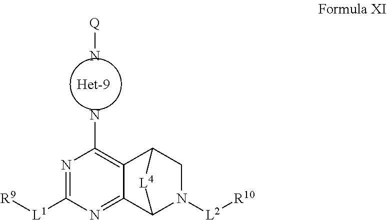

Wherein

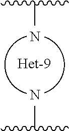

is selected from the following moieties:

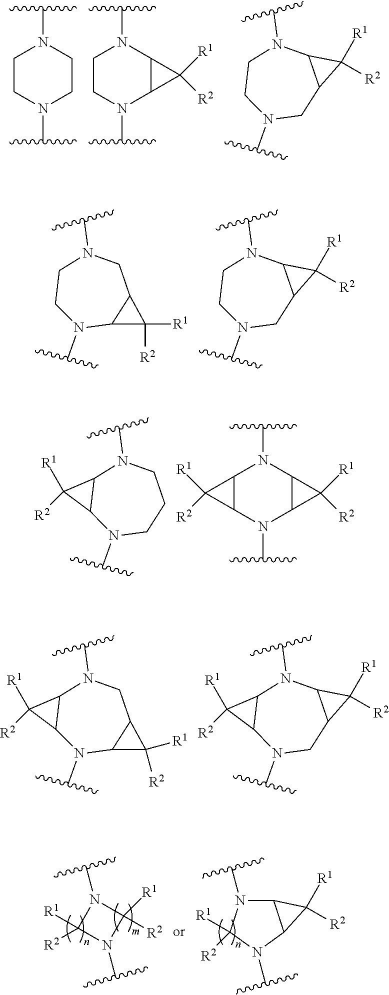

Q, n, m, L, L², R¹, R², R⁹ and R¹⁰ are defined as above;

L⁴ is —CR²¹R²²—, —(CR²¹R²²)₂—, O, S, NR²¹, NC(O)NR²¹, or NS(O)₂NR²¹R²².

R²¹ and R²² are independently selected from hydrogen, C₁₋₆alkyl, C₃₋₆cycloalkyl;

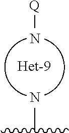

can also be selected from the following moieties:

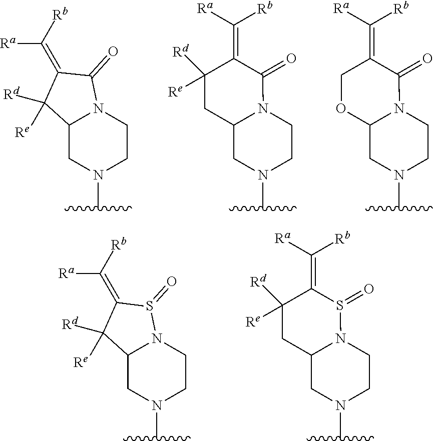

R^{a}, R^{b}, R^{e} and R^{d} are defined as above.

In some embodiments, the invention also provided herein are a stereoisomer, an enantiomer, an atropoisomeric or a pharmaceutically acceptable salt of any of the compound of Formula I to Formula XI described above.

In some embodiments, a compound of Formula I to Formula XI is selected from a stereoisomer, an enantiomer, or an atropoisomeric or a pharmaceutically acceptable salt thereof.

In some embodiments, a compound of Formula I to Formula XI or a stereoisomer, an enantiomer, or an atropisomer of them, or a pharmaceutically acceptable salt thereof is selected from the following compounds:

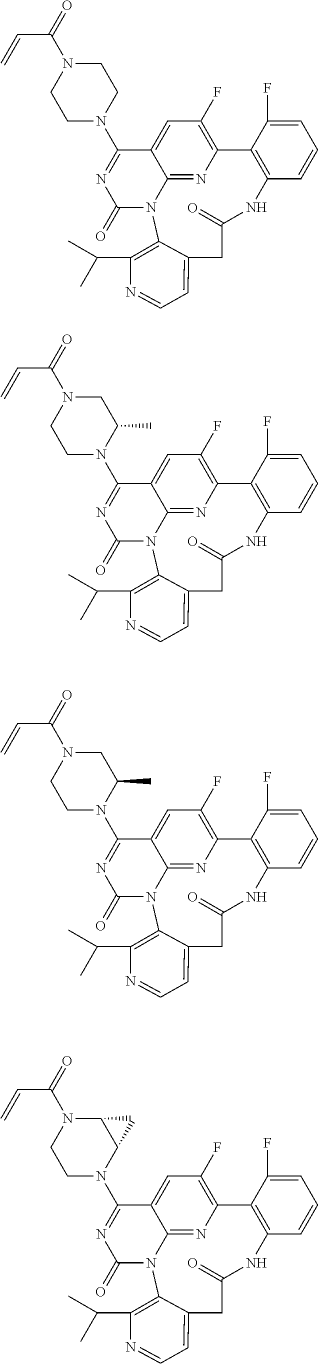 [SMILES: C=CC(=O)N1CCN(c2nc(=O)n3c4nc(c(F)cc24)-c2c(F)cccc2NC(=O)Cc2ccnc(C(C)C)c2-3)CC1 ; C=CC(=O)N1CCN(c2nc(=O)n3c4nc(c(F)cc24)-c2c(F)cccc2NC(=O)Cc2ccnc(C(C)C)c2-3)[C@@H](C)C1 ; C=CC(=O)N1CCN(c2nc(=O)n3c4nc(c(F)cc24)-c2c(F)cccc2NC(=O)Cc2ccnc(C(C)C)c2-3)[C@H](C)C1 ; C=CC(=O)N1CCN(c2nc(=O)n3c4nc(c(F)cc24)-c2c(F)cccc2NC(=O)Cc2ccnc(C(C)C)c2-3)[C@H]2C[C@H]21]

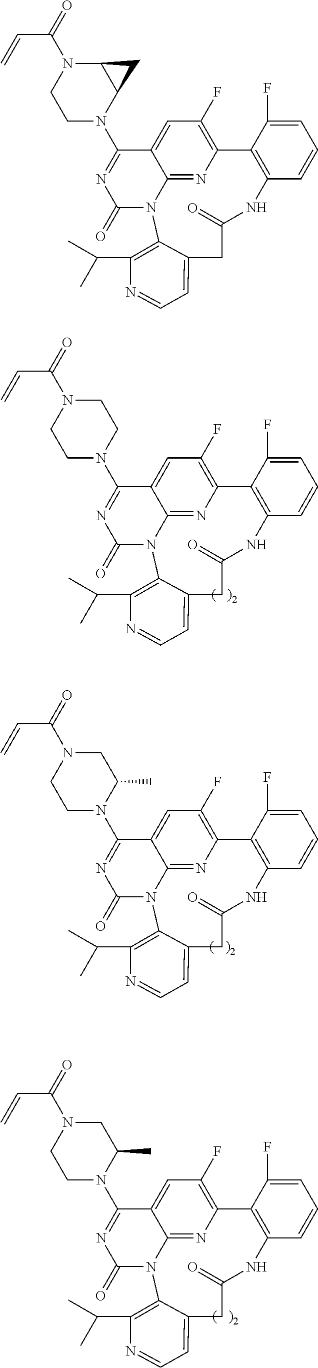

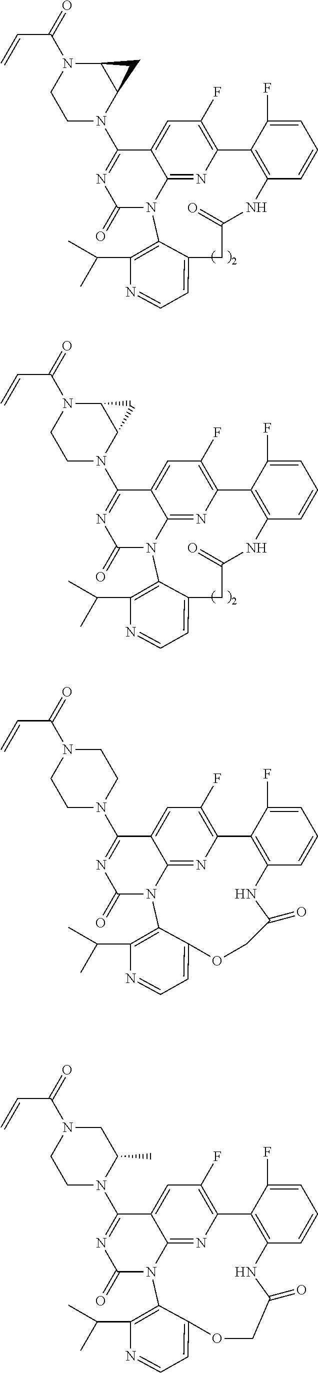

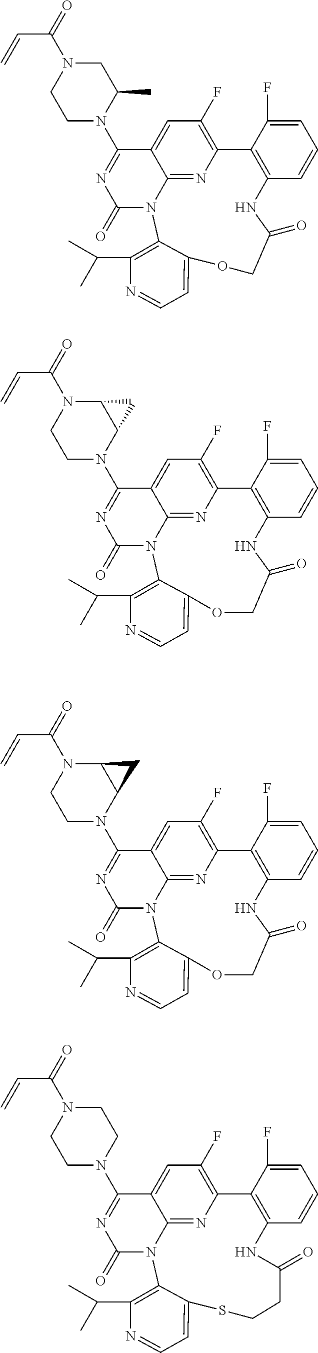 [SMILES: C=CC(=O)N1CCN(c2nc(=O)n3c4nc(c(F)cc24)-c2c(F)cccc2NC(=O)COc2ccnc(C(C)C)c2-3)[C@H](C)C1 ; C=CC(=O)N1CCN(c2nc(=O)n3c4nc(c(F)cc24)-c2c(F)cccc2NC(=O)COc2ccnc(C(C)C)c2-3)[C@H]2C[C@H]21 ; C=CC(=O)N1CCN(c2nc(=O)n3c4nc(c(F)cc24)-c2c(F)cccc2NC(=O)COc2ccnc(C(C)C)c2-3)[C@@H]2C[C@@H]21 ; C=CC(=O)N1CCN(c2nc(=O)n3c4nc(c(F)cc24)-c2c(F)cccc2NC(=O)CCSc2ccnc(C(C)C)c2-3)CC1]

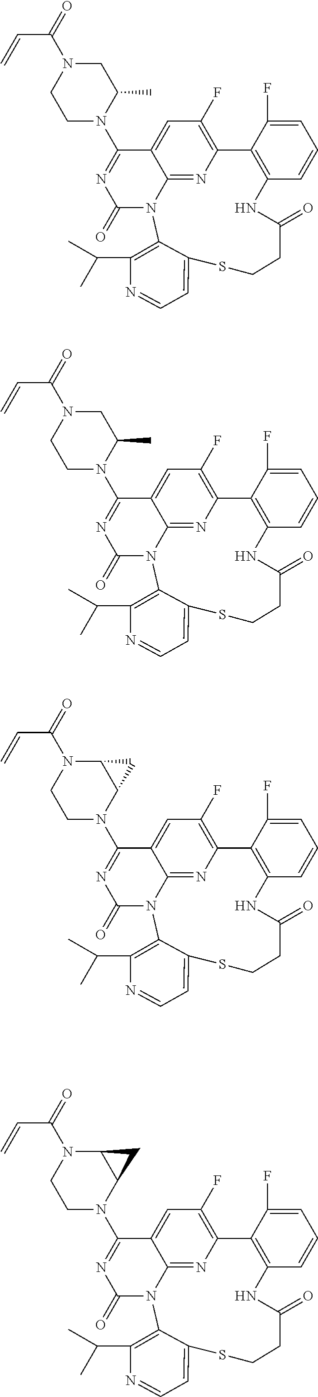 [SMILES: C=CC(=O)N1CCN(c2nc(=O)n3c4nc(c(F)cc24)-c2c(F)cccc2NC(=O)CCSc2ccnc(C(C)C)c2-3)[C@@H](C)C1 ; C=CC(=O)N1CCN(c2nc(=O)n3c4nc(c(F)cc24)-c2c(F)cccc2NC(=O)CCSc2ccnc(C(C)C)c2-3)[C@H](C)C1 ; C=CC(=O)N1CCN(c2nc(=O)n3c4nc(c(F)cc24)-c2c(F)cccc2NC(=O)CCSc2ccnc(C(C)C)c2-3)[C@H]2C[C@H]21 ; C=CC(=O)N1CCN(c2nc(=O)n3c4nc(c(F)cc24)-c2c(F)cccc2NC(=O)CCSc2ccnc(C(C)C)c2-3)[C@@H]2C[C@@H]21]

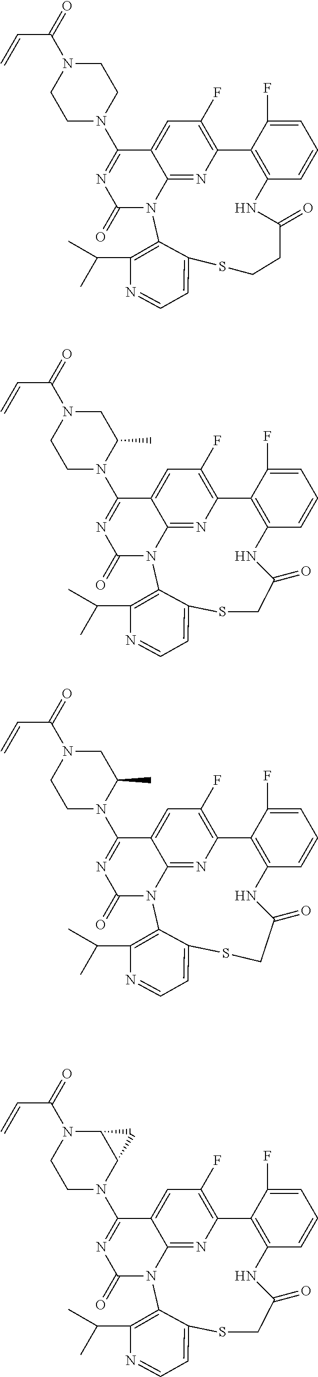 [SMILES: C=CC(=O)N1CCN(c2nc(=O)n3c4nc(c(F)cc24)-c2c(F)cccc2NC(=O)CCSc2ccnc(C(C)C)c2-3)CC1 ; C=CC(=O)N1CCN(c2nc(=O)n3c4nc(c(F)cc24)-c2c(F)cccc2NC(=O)CSc2ccnc(C(C)C)c2-3)[C@@H](C)C1 ; C=CC(=O)N1CCN(c2nc(=O)n3c4nc(c(F)cc24)-c2c(F)cccc2NC(=O)CSc2ccnc(C(C)C)c2-3)[C@H](C)C1 ; C=CC(=O)N1CCN(c2nc(=O)n3c4nc(c(F)cc24)-c2c(F)cccc2NC(=O)CSc2ccnc(C(C)C)c2-3)[C@H]2C[C@H]21]

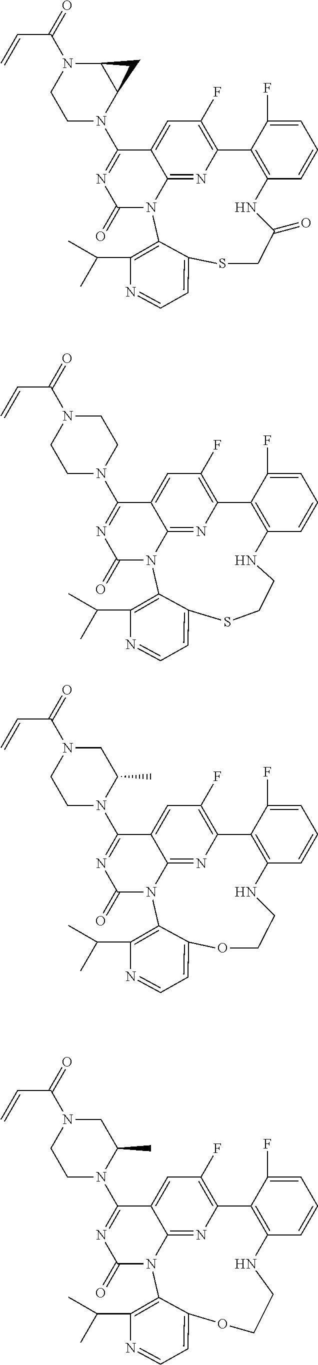 [SMILES: C=CC(=O)N1CCN(c2nc(=O)n3c4nc(c(F)cc24)-c2c(F)cccc2NC(=O)CSc2ccnc(C(C)C)c2-3)[C@@H]2C[C@@H]21 ; C=CC(=O)N1CCN(c2nc(=O)n3c4nc(c(F)cc24)-c2c(F)cccc2NCCSc2ccnc(C(C)C)c2-3)CC1 ; C=CC(=O)N1CCN(c2nc(=O)n3c4nc(c(F)cc24)-c2c(F)cccc2NCCOc2ccnc(C(C)C)c2-3)[C@@H](C)C1 ; C=CC(=O)N1CCN(c2nc(=O)n3c4nc(c(F)cc24)-c2c(F)cccc2NCCOc2ccnc(C(C)C)c2-3)[C@H](C)C1]

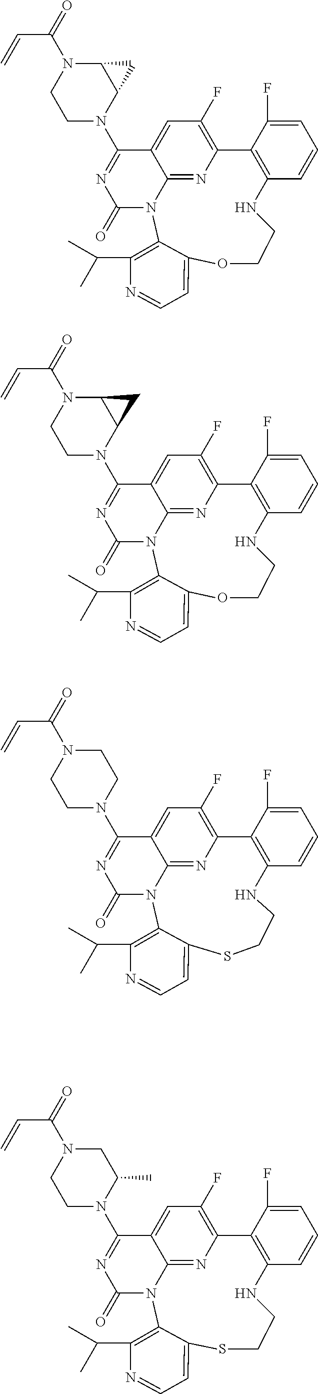 [SMILES: C=CC(=O)N1CCN(c2nc(=O)n3c4nc(c(F)cc24)-c2c(F)cccc2NCCOc2ccnc(C(C)C)c2-3)[C@H]2C[C@H]21 ; C=CC(=O)N1CCN(c2nc(=O)n3c4nc(c(F)cc24)-c2c(F)cccc2NCCOc2ccnc(C(C)C)c2-3)[C@@H]2C[C@@H]21 ; C=CC(=O)N1CCN(c2nc(=O)n3c4nc(c(F)cc24)-c2c(F)cccc2NCCSc2ccnc(C(C)C)c2-3)CC1 ; C=CC(=O)N1CCN(c2nc(=O)n3c4nc(c(F)cc24)-c2c(F)cccc2NCCSc2ccnc(C(C)C)c2-3)[C@@H](C)C1]

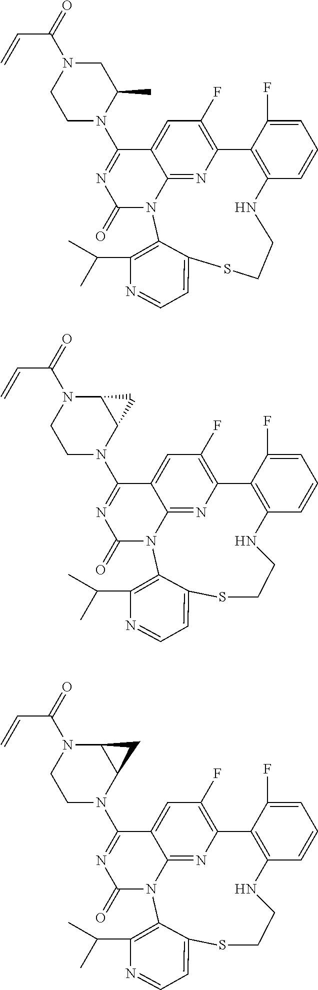 [SMILES: C=CC(=O)N1CCN(c2nc(=O)n3c4nc(c(F)cc24)-c2c(F)cccc2NCCSc2ccnc(C(C)C)c2-3)[C@H](C)C1 ; C=CC(=O)N1CCN(c2nc(=O)n3c4nc(c(F)cc24)-c2c(F)cccc2NCCSc2ccnc(C(C)C)c2-3)[C@H]2C[C@H]21 ; C=CC(=O)N1CCN(c2nc(=O)n3c4nc(c(F)cc24)-c2c(F)cccc2NCCSc2ccnc(C(C)C)c2-3)[C@@H]2C[C@@H]21]

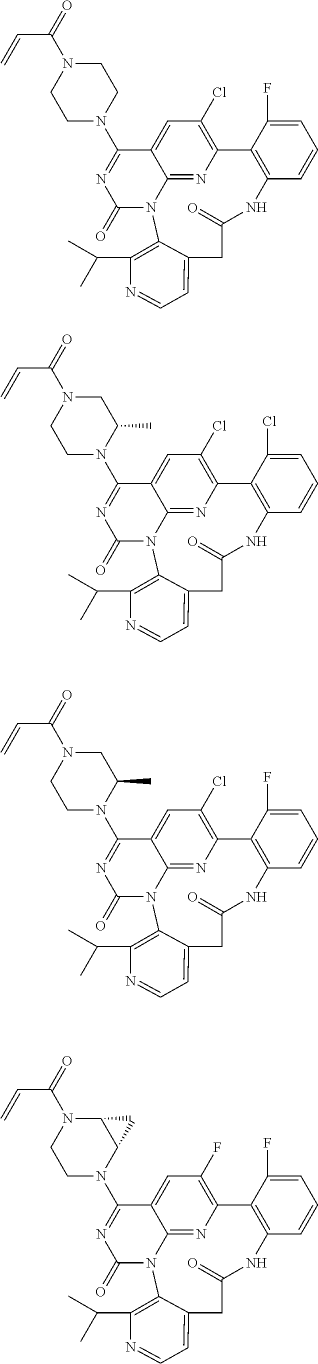 [SMILES: C=CC(=O)N1CCN(c2nc(=O)n3c4nc(c(Cl)cc24)-c2c(F)cccc2NC(=O)Cc2ccnc(C(C)C)c2-3)CC1 ; C=CC(=O)N1CCN(c2nc(=O)n3c4nc(c(Cl)cc24)-c2c(Cl)cccc2NC(=O)Cc2ccnc(C(C)C)c2-3)[C@@H](C)C1 ; C=CC(=O)N1CCN(c2nc(=O)n3c4nc(c(Cl)cc24)-c2c(F)cccc2NC(=O)Cc2ccnc(C(C)C)c2-3)[C@H](C)C1 ; C=CC(=O)N1CCN(c2nc(=O)n3c4nc(c(F)cc24)-c2c(F)cccc2NC(=O)Cc2ccnc(C(C)C)c2-3)[C@H]2C[C@H]21]

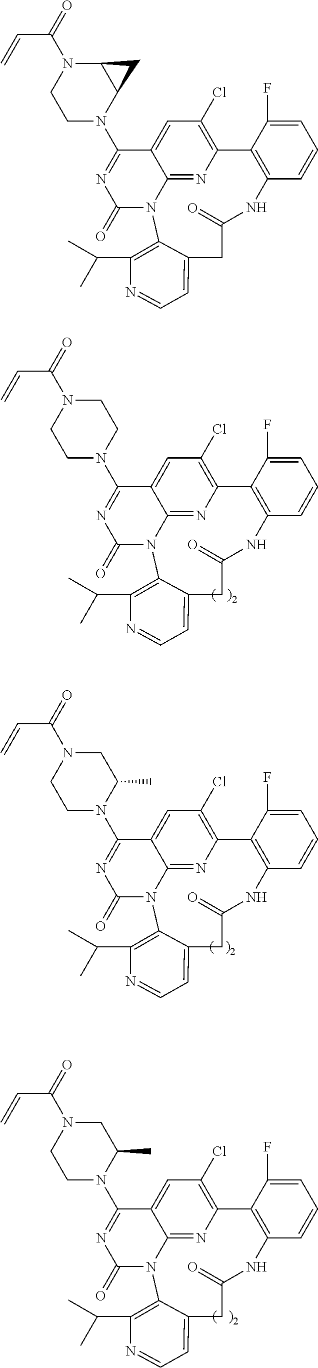

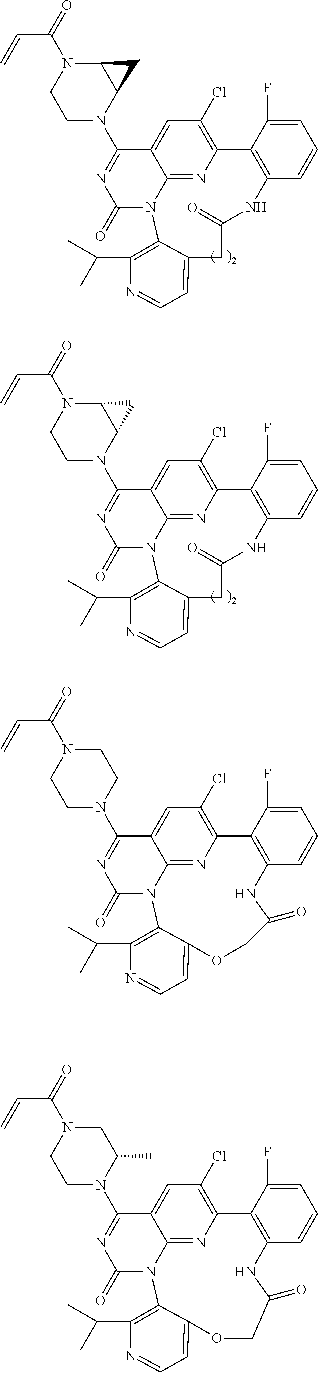

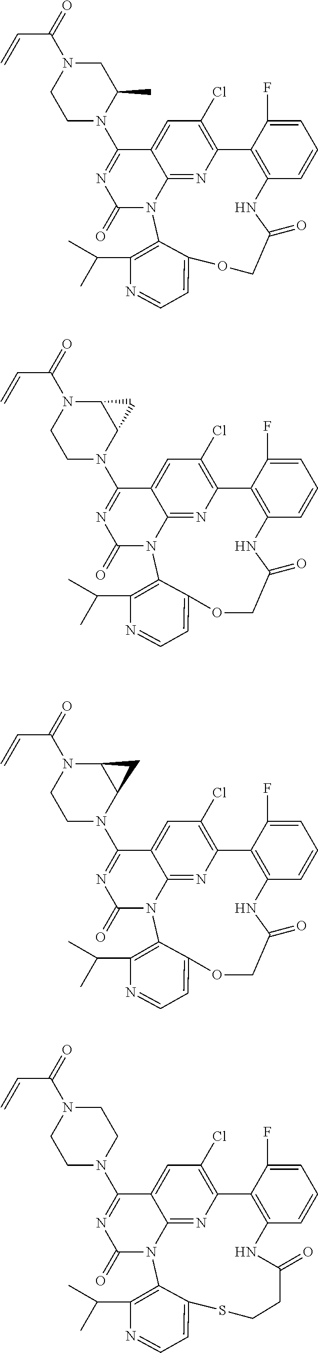 [SMILES: C=CC(=O)N1CCN(c2nc(=O)n3c4nc(c(Cl)cc24)-c2c(F)cccc2NC(=O)COc2ccnc(C(C)C)c2-3)[C@H](C)C1 ; C=CC(=O)N1CCN(c2nc(=O)n3c4nc(c(Cl)cc24)-c2c(F)cccc2NC(=O)COc2ccnc(C(C)C)c2-3)[C@H]2C[C@H]21 ; C=CC(=O)N1CCN(c2nc(=O)n3c4nc(c(Cl)cc24)-c2c(F)cccc2NC(=O)COc2ccnc(C(C)C)c2-3)[C@@H]2C[C@@H]21 ; C=CC(=O)N1CCN(c2nc(=O)n3c4nc(c(Cl)cc24)-c2c(F)cccc2NC(=O)CCSc2ccnc(C(C)C)c2-3)CC1]

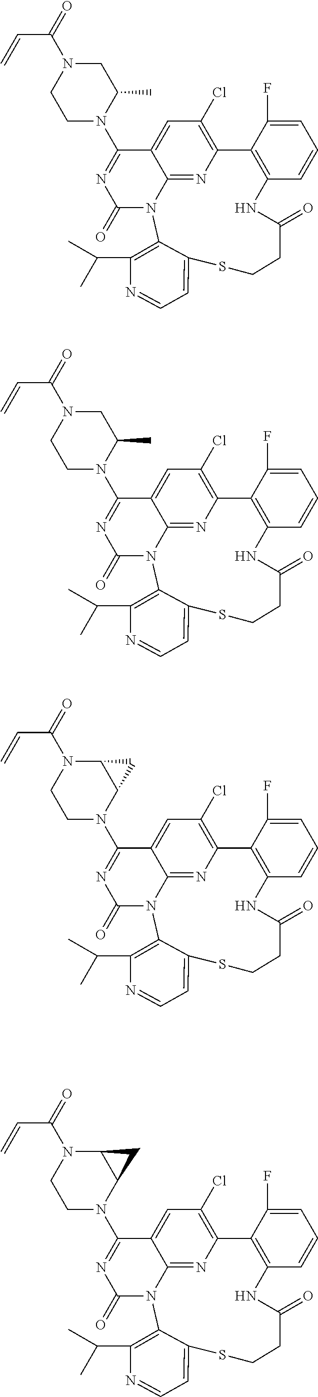 [SMILES: C=CC(=O)N1CCN(c2nc(=O)n3c4nc(c(Cl)cc24)-c2c(F)cccc2NC(=O)CCSc2ccnc(C(C)C)c2-3)[C@@H](C)C1 ; C=CC(=O)N1CCN(c2nc(=O)n3c4nc(c(Cl)cc24)-c2c(F)cccc2NC(=O)CCSc2ccnc(C(C)C)c2-3)[C@H](C)C1 ; C=CC(=O)N1CCN(c2nc(=O)n3c4nc(c(Cl)cc24)-c2c(F)cccc2NC(=O)CCSc2ccnc(C(C)C)c2-3)[C@H]2C[C@H]21 ; C=CC(=O)N1CCN(c2nc(=O)n3c4nc(c(Cl)cc24)-c2c(F)cccc2NC(=O)CCSc2ccnc(C(C)C)c2-3)[C@@H]2C[C@@H]21]

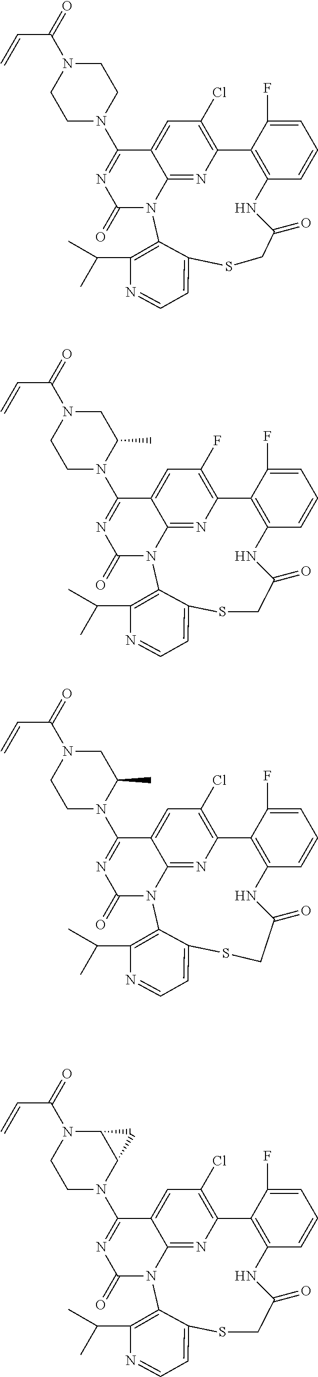 [SMILES: C=CC(=O)N1CCN(c2nc(=O)n3c4nc(c(Cl)cc24)-c2c(F)cccc2NC(=O)CSc2ccnc(C(C)C)c2-3)CC1 ; C=CC(=O)N1CCN(c2nc(=O)n3c4nc(c(F)cc24)-c2c(F)cccc2NC(=O)CSc2ccnc(C(C)C)c2-3)[C@@H](C)C1 ; C=CC(=O)N1CCN(c2nc(=O)n3c4nc(c(Cl)cc24)-c2c(F)cccc2NC(=O)CSc2ccnc(C(C)C)c2-3)[C@H](C)C1 ; C=CC(=O)N1CCN(c2nc(=O)n3c4nc(c(Cl)cc24)-c2c(F)cccc2NC(=O)CSc2ccnc(C(C)C)c2-3)[C@H]2C[C@H]21]

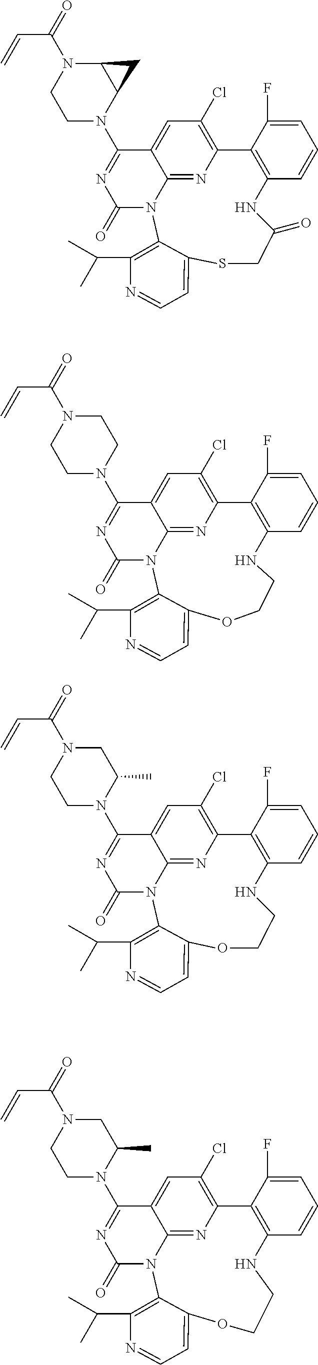 [SMILES: C=CC(=O)N1CCN(c2nc(=O)n3c4nc(c(Cl)cc24)-c2c(F)cccc2NC(=O)CSc2ccnc(C(C)C)c2-3)[C@@H]2C[C@@H]21 ; C=CC(=O)N1CCN(c2nc(=O)n3c4nc(c(Cl)cc24)-c2c(F)cccc2NCCOc2ccnc(C(C)C)c2-3)CC1 ; C=CC(=O)N1CCN(c2nc(=O)n3c4nc(c(Cl)cc24)-c2c(F)cccc2NCCOc2ccnc(C(C)C)c2-3)[C@@H](C)C1 ; C=CC(=O)N1CCN(c2nc(=O)n3c4nc(c(Cl)cc24)-c2c(F)cccc2NCCOc2ccnc(C(C)C)c2-3)[C@H](C)C1]

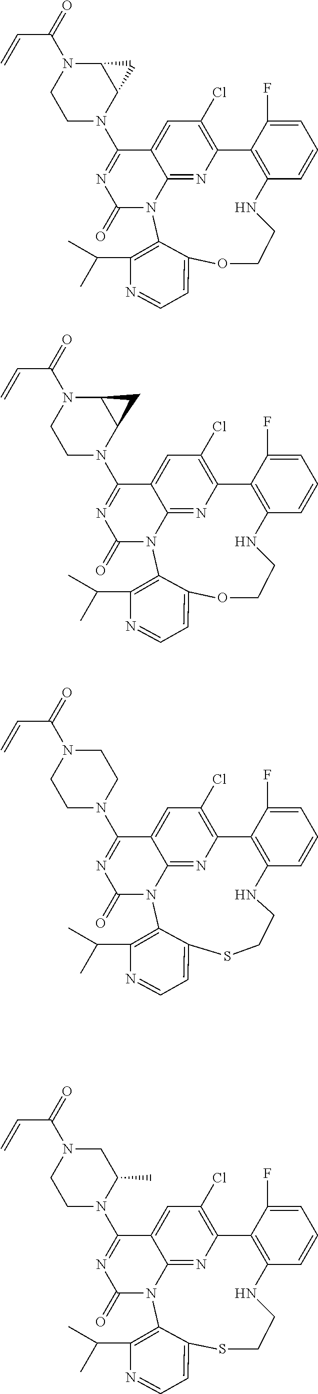 [SMILES: C=CC(=O)N1CCN(c2nc(=O)n3c4nc(c(Cl)cc24)-c2c(F)cccc2NCCOc2ccnc(C(C)C)c2-3)[C@H]2C[C@H]21 ; C=CC(=O)N1CCN(c2nc(=O)n3c4nc(c(Cl)cc24)-c2c(F)cccc2NCCOc2ccnc(C(C)C)c2-3)[C@@H]2C[C@@H]21 ; C=CC(=O)N1CCN(c2nc(=O)n3c4nc(c(Cl)cc24)-c2c(F)cccc2NCCSc2ccnc(C(C)C)c2-3)CC1 ; C=CC(=O)N1CCN(c2nc(=O)n3c4nc(c(Cl)cc24)-c2c(F)cccc2NCCSc2ccnc(C(C)C)c2-3)[C@@H](C)C1]

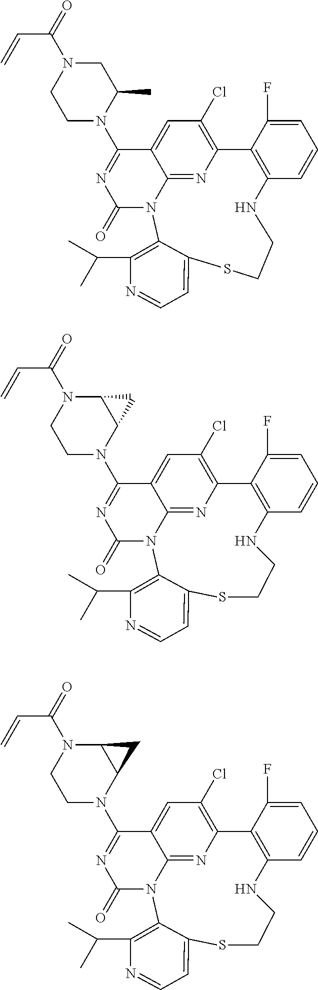 [SMILES: C=CC(=O)N1CCN(c2nc(=O)n3c4nc(c(Cl)cc24)-c2c(F)cccc2NCCSc2ccnc(C(C)C)c2-3)[C@H](C)C1 ; C=CC(=O)N1CCN(c2nc(=O)n3c4nc(c(Cl)cc24)-c2c(F)cccc2NCCSc2ccnc(C(C)C)c2-3)[C@H]2C[C@H]21 ; C=CC(=O)N1CCN(c2nc(=O)n3c4nc(c(Cl)cc24)-c2c(F)cccc2NCCSc2ccnc(C(C)C)c2-3)[C@@H]2C[C@@H]21]

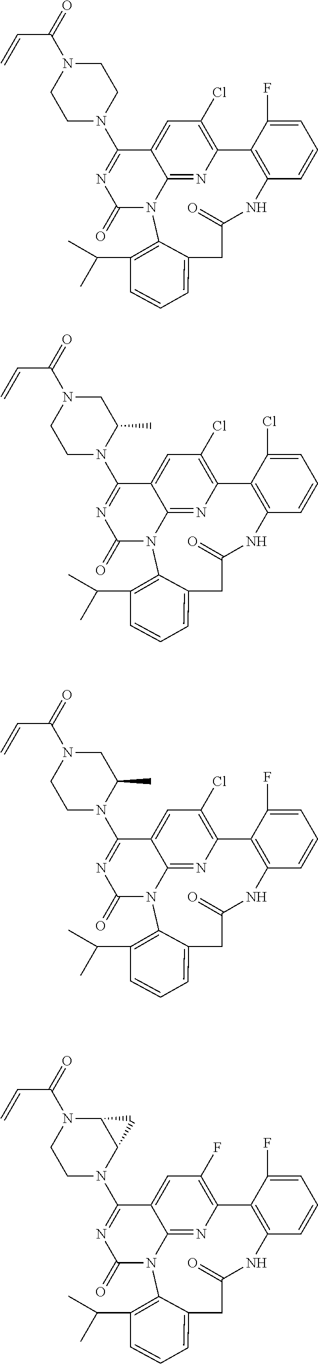 [SMILES: C=CC(=O)N1CCN(c2nc(=O)n3c4nc(c(Cl)cc24)-c2c(F)cccc2NC(=O)Cc2cccc(C(C)C)c2-3)CC1 ; C=CC(=O)N1CCN(c2nc(=O)n3c4nc(c(Cl)cc24)-c2c(Cl)cccc2NC(=O)Cc2cccc(C(C)C)c2-3)[C@@H](C)C1 ; C=CC(=O)N1CCN(c2nc(=O)n3c4nc(c(Cl)cc24)-c2c(F)cccc2NC(=O)Cc2cccc(C(C)C)c2-3)[C@H](C)C1 ; C=CC(=O)N1CCN(c2nc(=O)n3c4nc(c(F)cc24)-c2c(F)cccc2NC(=O)Cc2cccc(C(C)C)c2-3)[C@H]2C[C@H]21]

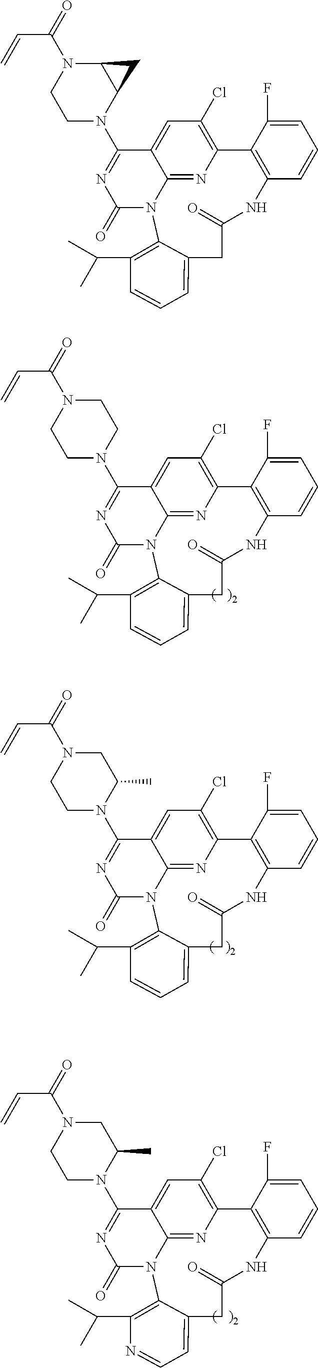

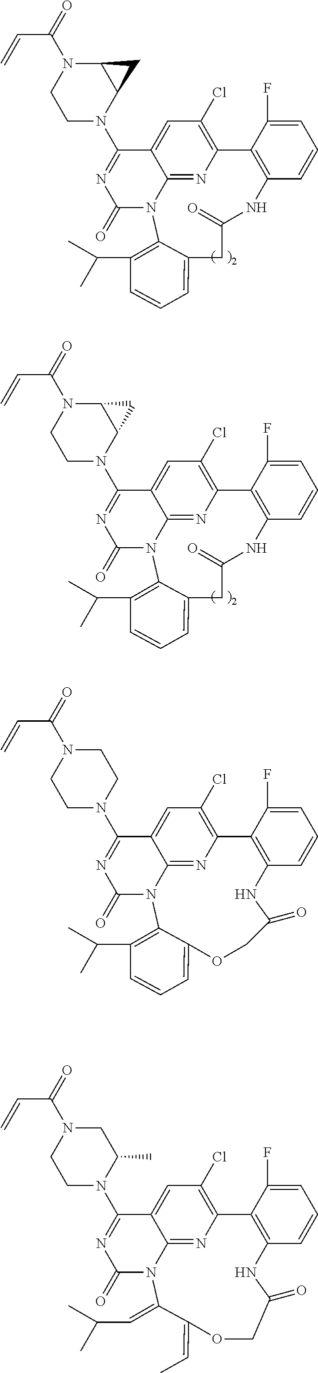

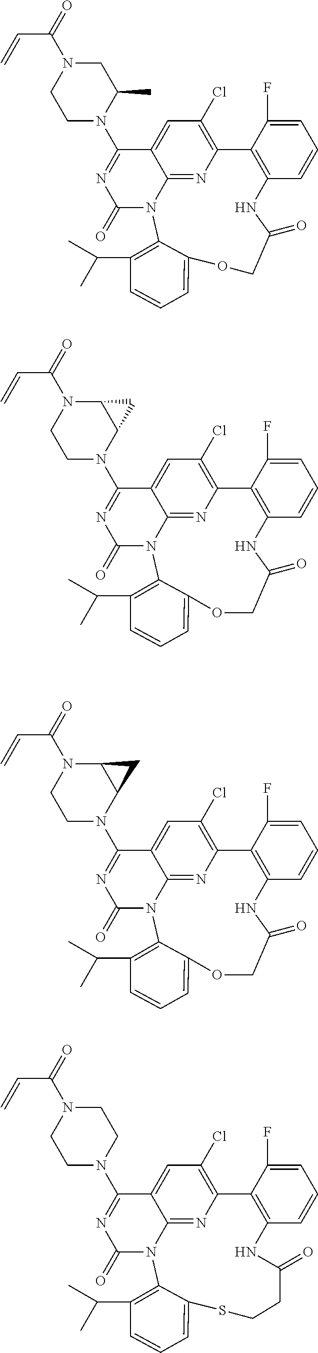 [SMILES: C=CC(=O)N1CCN(c2nc(=O)n3c4nc(c(Cl)cc24)-c2c(F)cccc2NC(=O)COc2cccc(C(C)C)c2-3)[C@H](C)C1 ; C=CC(=O)N1CCN(c2nc(=O)n3c4nc(c(Cl)cc24)-c2c(F)cccc2NC(=O)COc2cccc(C(C)C)c2-3)[C@H]2C[C@H]21 ; C=CC(=O)N1CCN(c2nc(=O)n3c4nc(c(Cl)cc24)-c2c(F)cccc2NC(=O)COc2cccc(C(C)C)c2-3)[C@@H]2C[C@@H]21 ; C=CC(=O)N1CCN(c2nc(=O)n3c4nc(c(Cl)cc24)-c2c(F)cccc2NC(=O)CCSc2cccc(C(C)C)c2-3)CC1]

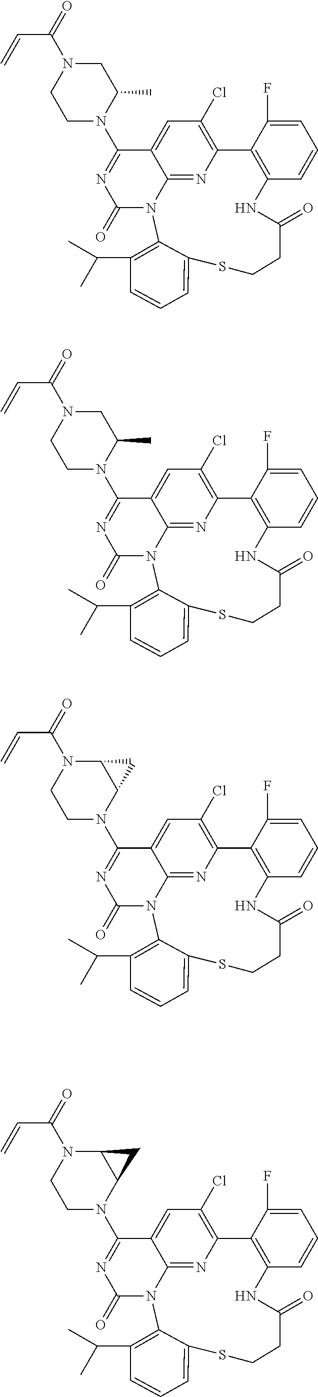 [SMILES: C=CC(=O)N1CCN(c2nc(=O)n3c4nc(c(Cl)cc24)-c2c(F)cccc2NC(=O)CCSc2cccc(C(C)C)c2-3)[C@@H](C)C1 ; C=CC(=O)N1CCN(c2nc(=O)n3c4nc(c(Cl)cc24)-c2c(F)cccc2NC(=O)CCSc2cccc(C(C)C)c2-3)[C@H](C)C1 ; C=CC(=O)N1CCN(c2nc(=O)n3c4nc(c(Cl)cc24)-c2c(F)cccc2NC(=O)CCSc2cccc(C(C)C)c2-3)[C@H]2C[C@H]21 ; C=CC(=O)N1CCN(c2nc(=O)n3c4nc(c(Cl)cc24)-c2c(F)cccc2NC(=O)CCSc2cccc(C(C)C)c2-3)[C@@H]2C[C@@H]21]

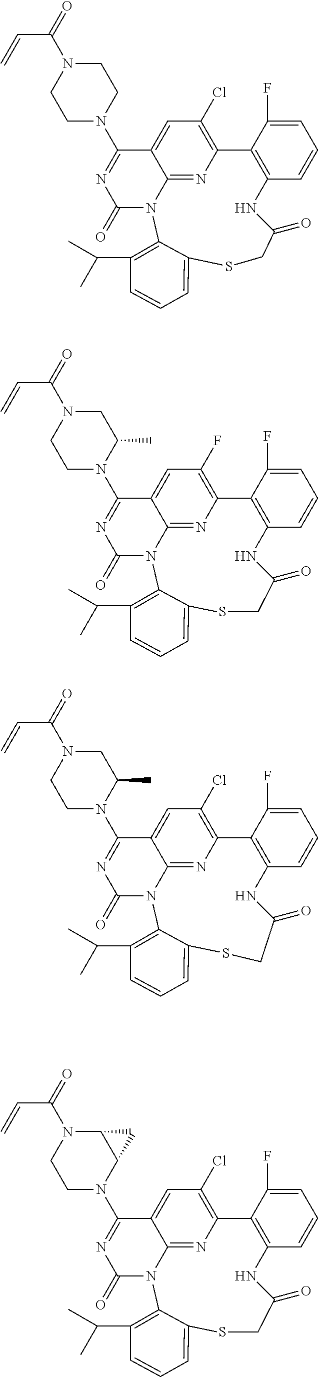 [SMILES: C=CC(=O)N1CCN(c2nc(=O)n3c4nc(c(Cl)cc24)-c2c(F)cccc2NC(=O)CSc2cccc(C(C)C)c2-3)CC1 ; C=CC(=O)N1CCN(c2nc(=O)n3c4nc(c(F)cc24)-c2c(F)cccc2NC(=O)CSc2cccc(C(C)C)c2-3)[C@@H](C)C1 ; C=CC(=O)N1CCN(c2nc(=O)n3c4nc(c(Cl)cc24)-c2c(F)cccc2NC(=O)CSc2cccc(C(C)C)c2-3)[C@H](C)C1 ; C=CC(=O)N1CCN(c2nc(=O)n3c4nc(c(Cl)cc24)-c2c(F)cccc2NC(=O)CSc2cccc(C(C)C)c2-3)[C@H]2C[C@H]21]

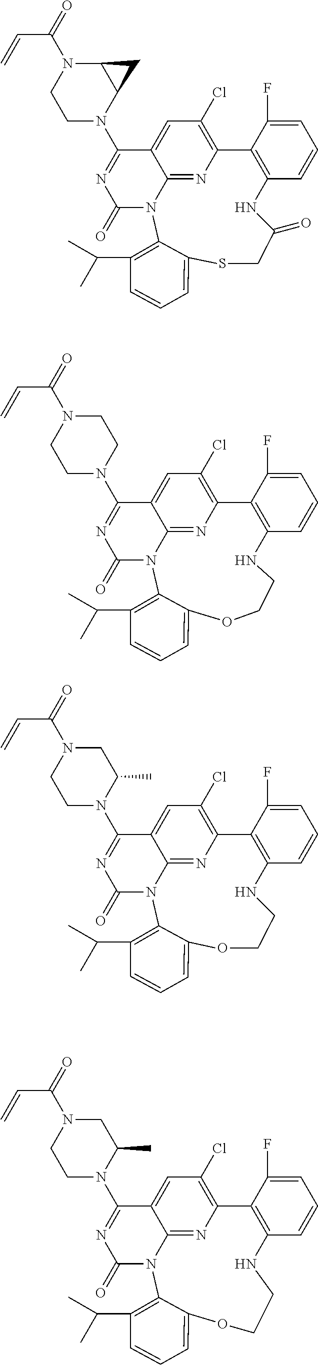 [SMILES: C=CC(=O)N1CCN(c2nc(=O)n3c4nc(c(Cl)cc24)-c2c(F)cccc2NC(=O)CSc2cccc(C(C)C)c2-3)[C@@H]2C[C@@H]21 ; C=CC(=O)N1CCN(c2nc(=O)n3c4nc(c(Cl)cc24)-c2c(F)cccc2NCCOc2cccc(C(C)C)c2-3)CC1 ; C=CC(=O)N1CCN(c2nc(=O)n3c4nc(c(Cl)cc24)-c2c(F)cccc2NCCOc2cccc(C(C)C)c2-3)[C@@H](C)C1 ; C=CC(=O)N1CCN(c2nc(=O)n3c4nc(c(Cl)cc24)-c2c(F)cccc2NCCOc2cccc(C(C)C)c2-3)[C@H](C)C1]

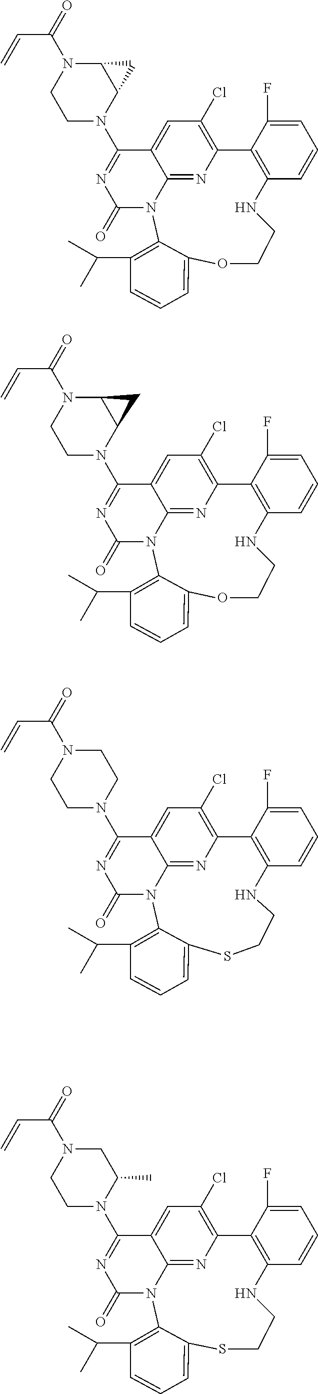 [SMILES: C=CC(=O)N1CCN(c2nc(=O)n3c4nc(c(Cl)cc24)-c2c(F)cccc2NCCOc2cccc(C(C)C)c2-3)[C@H]2C[C@H]21 ; C=CC(=O)N1CCN(c2nc(=O)n3c4nc(c(Cl)cc24)-c2c(F)cccc2NCCOc2cccc(C(C)C)c2-3)[C@@H]2C[C@@H]21 ; C=CC(=O)N1CCN(c2nc(=O)n3c4nc(c(Cl)cc24)-c2c(F)cccc2NCCSc2cccc(C(C)C)c2-3)CC1 ; C=CC(=O)N1CCN(c2nc(=O)n3c4nc(c(Cl)cc24)-c2c(F)cccc2NCCSc2cccc(C(C)C)c2-3)[C@@H](C)C1]

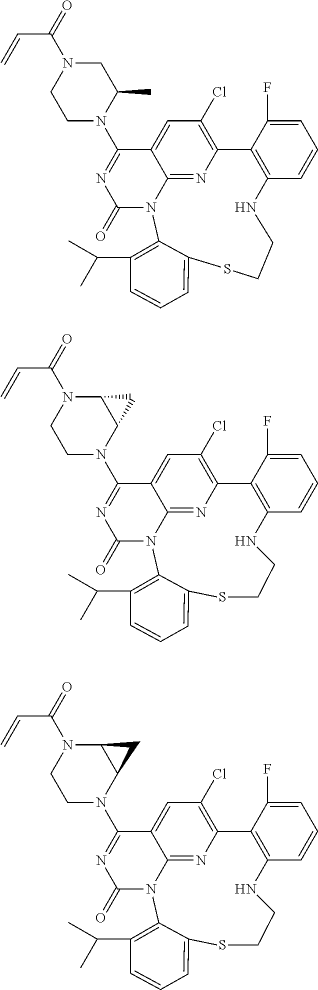 [SMILES: C=CC(=O)N1CCN(c2nc(=O)n3c4nc(c(Cl)cc24)-c2c(F)cccc2NCCSc2cccc(C(C)C)c2-3)[C@H](C)C1 ; C=CC(=O)N1CCN(c2nc(=O)n3c4nc(c(Cl)cc24)-c2c(F)cccc2NCCSc2cccc(C(C)C)c2-3)[C@H]2C[C@H]21 ; C=CC(=O)N1CCN(c2nc(=O)n3c4nc(c(Cl)cc24)-c2c(F)cccc2NCCSc2cccc(C(C)C)c2-3)[C@@H]2C[C@@H]21]

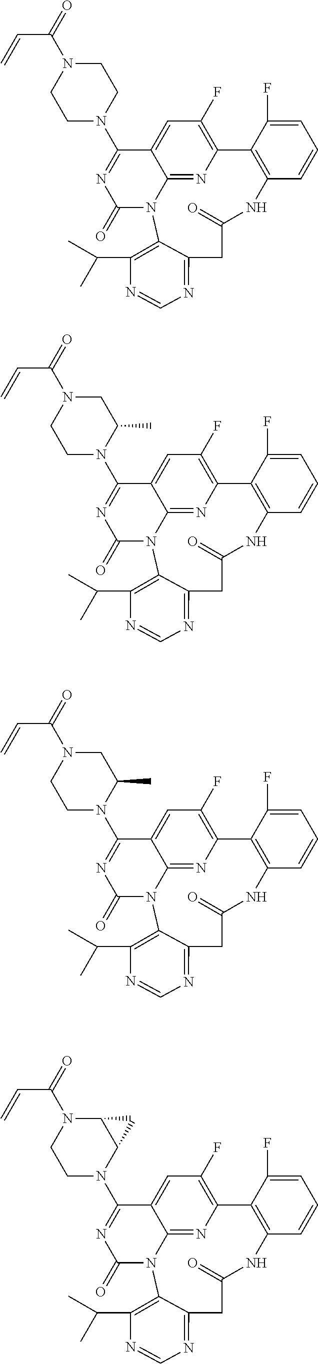 [SMILES: C=CC(=O)N1CCN(c2nc(=O)n3c4nc(c(F)cc24)-c2c(F)cccc2NC(=O)Cc2ncnc(C(C)C)c2-3)CC1 ; C=CC(=O)N1CCN(c2nc(=O)n3c4nc(c(F)cc24)-c2c(F)cccc2NC(=O)Cc2ncnc(C(C)C)c2-3)[C@@H](C)C1 ; C=CC(=O)N1CCN(c2nc(=O)n3c4nc(c(F)cc24)-c2c(F)cccc2NC(=O)Cc2ncnc(C(C)C)c2-3)[C@H](C)C1 ; C=CC(=O)N1CCN(c2nc(=O)n3c4nc(c(F)cc24)-c2c(F)cccc2NC(=O)Cc2ncnc(C(C)C)c2-3)[C@H]2C[C@H]21]

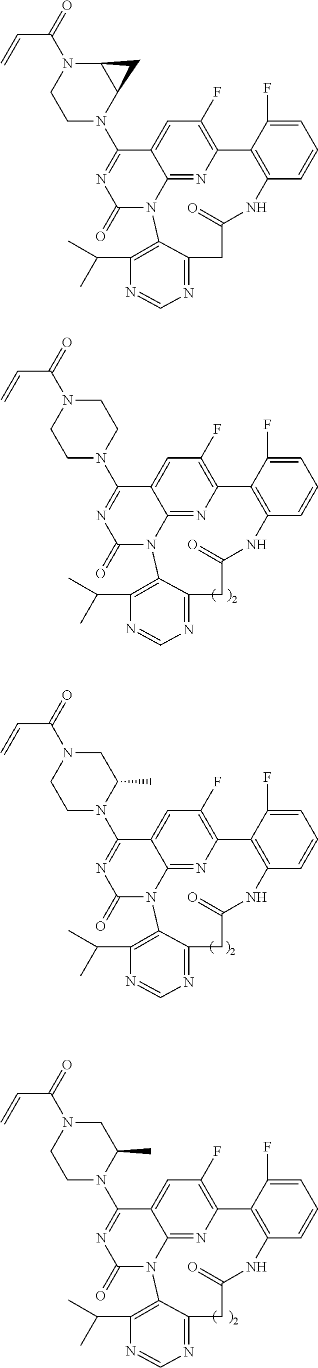

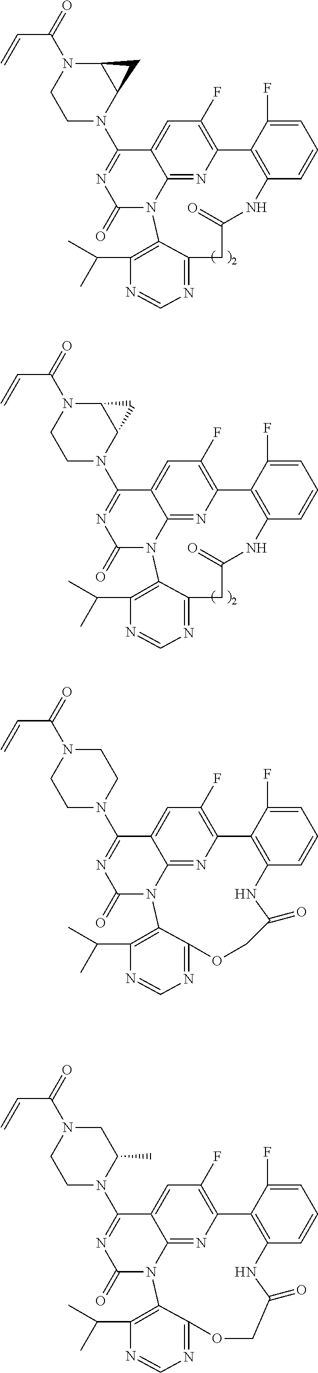

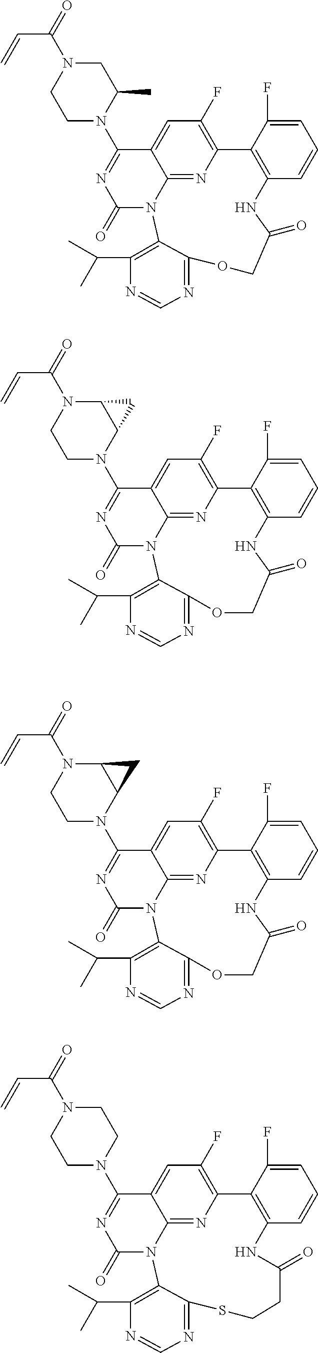 [SMILES: C=CC(=O)N1CCN(c2nc(=O)n3c4nc(c(F)cc24)-c2c(F)cccc2NC(=O)COc2ncnc(C(C)C)c2-3)[C@H](C)C1 ; C=CC(=O)N1CCN(c2nc(=O)n3c4nc(c(F)cc24)-c2c(F)cccc2NC(=O)COc2ncnc(C(C)C)c2-3)[C@H]2C[C@H]21 ; C=CC(=O)N1CCN(c2nc(=O)n3c4nc(c(F)cc24)-c2c(F)cccc2NC(=O)COc2ncnc(C(C)C)c2-3)[C@@H]2C[C@@H]21 ; C=CC(=O)N1CCN(c2nc(=O)n3c4nc(c(F)cc24)-c2c(F)cccc2NC(=O)CCSc2ncnc(C(C)C)c2-3)CC1]

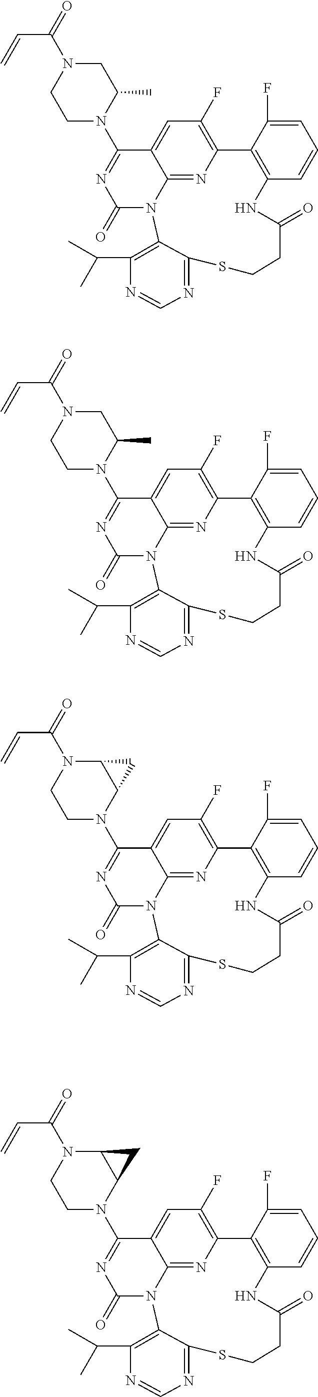 [SMILES: C=CC(=O)N1CCN(c2nc(=O)n3c4nc(c(F)cc24)-c2c(F)cccc2NC(=O)CCSc2ncnc(C(C)C)c2-3)[C@@H](C)C1 ; C=CC(=O)N1CCN(c2nc(=O)n3c4nc(c(F)cc24)-c2c(F)cccc2NC(=O)CCSc2ncnc(C(C)C)c2-3)[C@H](C)C1 ; C=CC(=O)N1CCN(c2nc(=O)n3c4nc(c(F)cc24)-c2c(F)cccc2NC(=O)CCSc2ncnc(C(C)C)c2-3)[C@H]2C[C@H]21 ; C=CC(=O)N1CCN(c2nc(=O)n3c4nc(c(F)cc24)-c2c(F)cccc2NC(=O)CCSc2ncnc(C(C)C)c2-3)[C@@H]2C[C@@H]21]

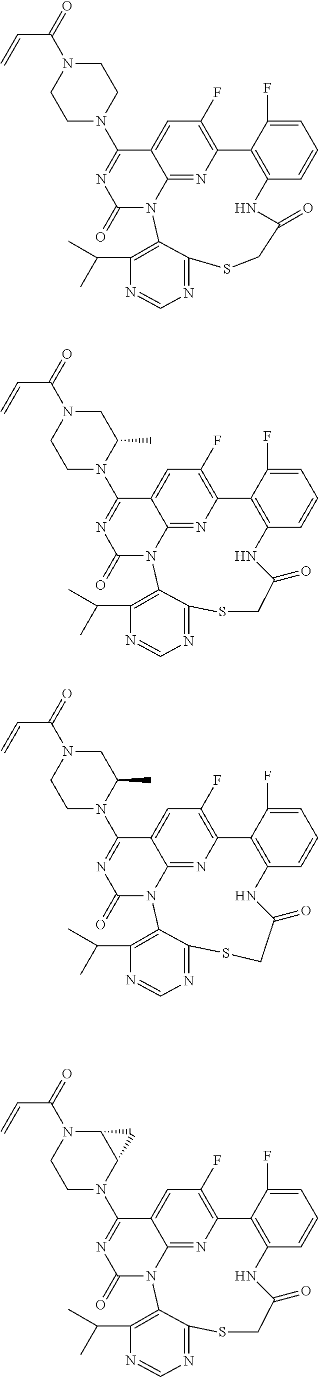 [SMILES: C=CC(=O)N1CCN(c2nc(=O)n3c4nc(c(F)cc24)-c2c(F)cccc2NC(=O)CSc2ncnc(C(C)C)c2-3)CC1 ; C=CC(=O)N1CCN(c2nc(=O)n3c4nc(c(F)cc24)-c2c(F)cccc2NC(=O)CSc2ncnc(C(C)C)c2-3)[C@@H](C)C1 ; C=CC(=O)N1CCN(c2nc(=O)n3c4nc(c(F)cc24)-c2c(F)cccc2NC(=O)CSc2ncnc(C(C)C)c2-3)[C@H](C)C1 ; C=CC(=O)N1CCN(c2nc(=O)n3c4nc(c(F)cc24)-c2c(F)cccc2NC(=O)CSc2ncnc(C(C)C)c2-3)[C@H]2C[C@H]21]

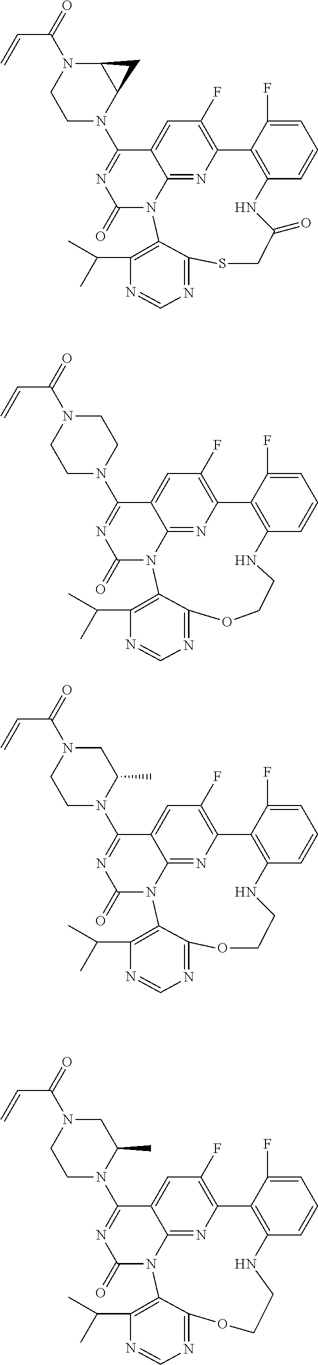 [SMILES: C=CC(=O)N1CCN(c2nc(=O)n3c4nc(c(F)cc24)-c2c(F)cccc2NC(=O)CSc2ncnc(C(C)C)c2-3)[C@@H]2C[C@@H]21 ; C=CC(=O)N1CCN(c2nc(=O)n3c4nc(c(F)cc24)-c2c(F)cccc2NCCOc2ncnc(C(C)C)c2-3)CC1 ; C=CC(=O)N1CCN(c2nc(=O)n3c4nc(c(F)cc24)-c2c(F)cccc2NCCOc2ncnc(C(C)C)c2-3)[C@@H](C)C1 ; C=CC(=O)N1CCN(c2nc(=O)n3c4nc(c(F)cc24)-c2c(F)cccc2NCCOc2ncnc(C(C)C)c2-3)[C@H](C)C1]

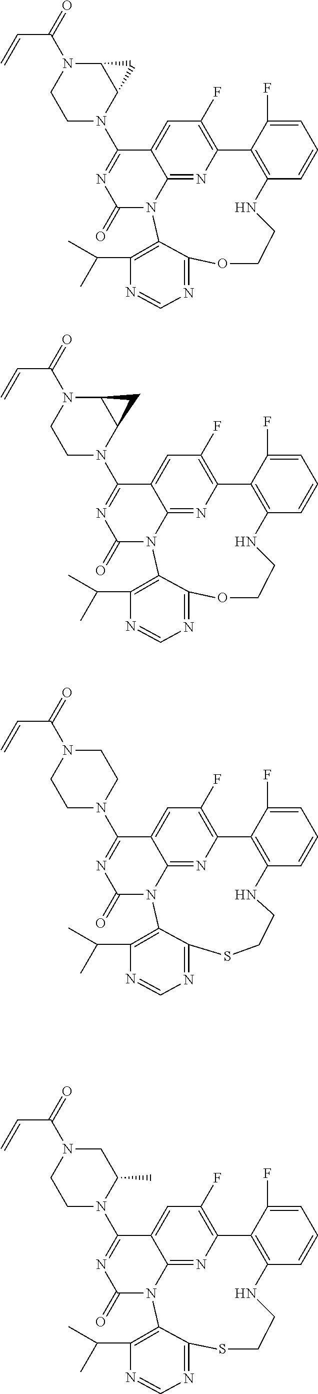 [SMILES: C=CC(=O)N1CCN(c2nc(=O)n3c4nc(c(F)cc24)-c2c(F)cccc2NCCOc2ncnc(C(C)C)c2-3)[C@H]2C[C@H]21 ; C=CC(=O)N1CCN(c2nc(=O)n3c4nc(c(F)cc24)-c2c(F)cccc2NCCOc2ncnc(C(C)C)c2-3)[C@@H]2C[C@@H]21 ; C=CC(=O)N1CCN(c2nc(=O)n3c4nc(c(F)cc24)-c2c(F)cccc2NCCSc2ncnc(C(C)C)c2-3)CC1 ; C=CC(=O)N1CCN(c2nc(=O)n3c4nc(c(F)cc24)-c2c(F)cccc2NCCSc2ncnc(C(C)C)c2-3)[C@@H](C)C1]

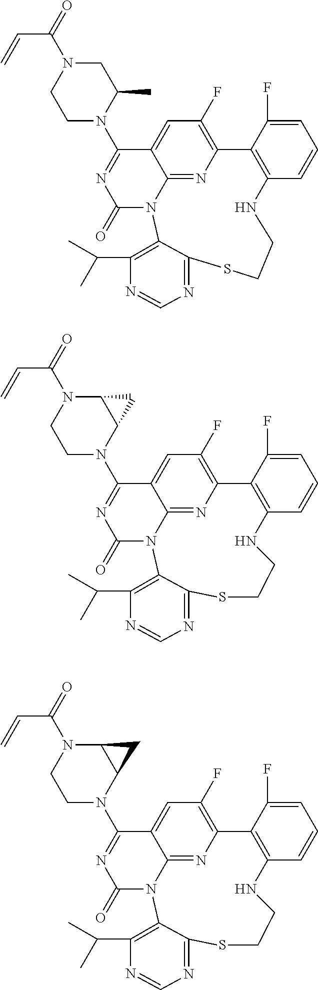 [SMILES: C=CC(=O)N1CCN(c2nc(=O)n3c4nc(c(F)cc24)-c2c(F)cccc2NCCSc2ncnc(C(C)C)c2-3)[C@H](C)C1 ; C=CC(=O)N1CCN(c2nc(=O)n3c4nc(c(F)cc24)-c2c(F)cccc2NCCSc2ncnc(C(C)C)c2-3)[C@H]2C[C@H]21 ; C=CC(=O)N1CCN(c2nc(=O)n3c4nc(c(F)cc24)-c2c(F)cccc2NCCSc2ncnc(C(C)C)c2-3)[C@@H]2C[C@@H]21]

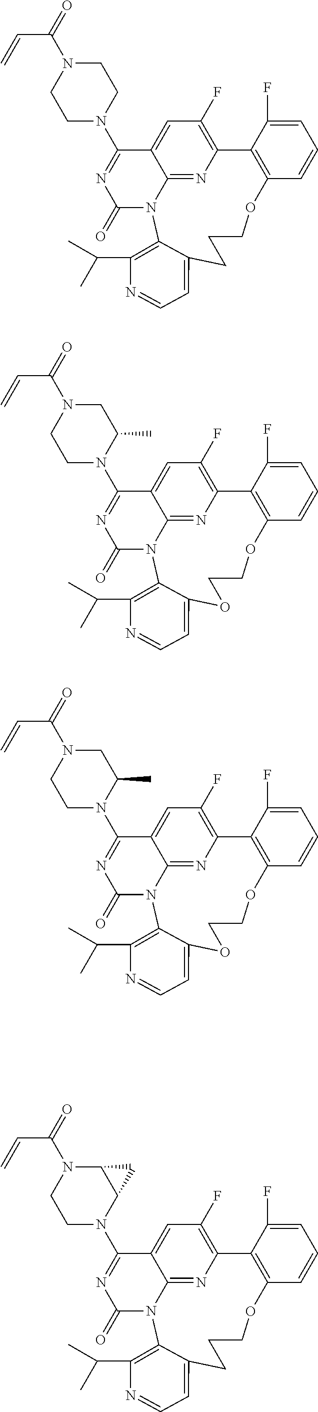 [SMILES: C=CC(=O)N1CCN(c2nc(=O)n3c4nc(c(F)cc24)-c2c(F)cccc2OCCCc2ccnc(C(C)C)c2-3)CC1 ; C=CC(=O)N1CCN(c2nc(=O)n3c4nc(c(F)cc24)-c2c(F)cccc2OCCOc2ccnc(C(C)C)c2-3)[C@@H](C)C1 ; C=CC(=O)N1CCN(c2nc(=O)n3c4nc(c(F)cc24)-c2c(F)cccc2OCCOc2ccnc(C(C)C)c2-3)[C@H](C)C1 ; C=CC(=O)N1CCN(c2nc(=O)n3c4nc(c(F)cc24)-c2c(F)cccc2OCCCc2ccnc(C(C)C)c2-3)[C@H]2C[C@H]21]

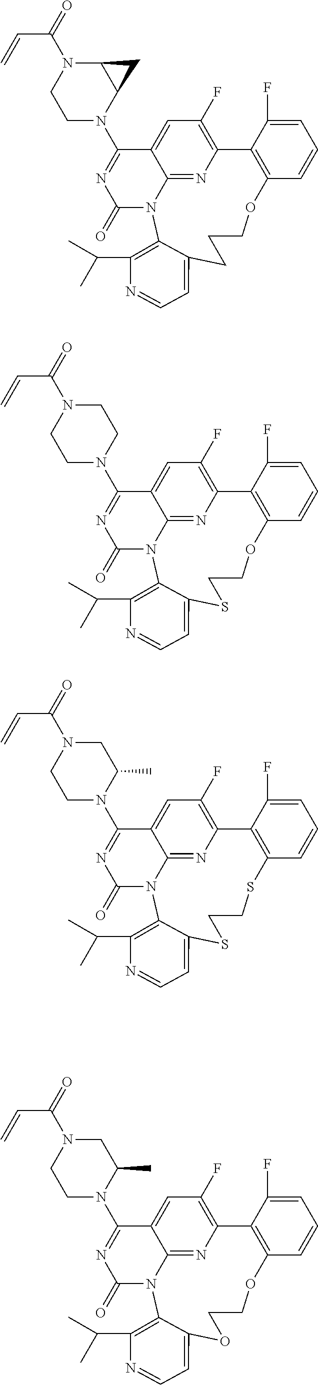 [SMILES: C=CC(=O)N1CCN(c2nc(=O)n3c4nc(c(F)cc24)-c2c(F)cccc2OCCCc2ccnc(C(C)C)c2-3)[C@@H]2C[C@@H]21 ; C=CC(=O)N1CCN(c2nc(=O)n3c4nc(c(F)cc24)-c2c(F)cccc2OCCSc2ccnc(C(C)C)c2-3)CC1 ; C=CC(=O)N1CCN(c2nc(=O)n3c4nc(c(F)cc24)-c2c(F)cccc2SCCSc2ccnc(C(C)C)c2-3)[C@@H](C)C1 ; C=CC(=O)N1CCN(c2nc(=O)n3c4nc(c(F)cc24)-c2c(F)cccc2OCCOc2ccnc(C(C)C)c2-3)[C@H](C)C1]

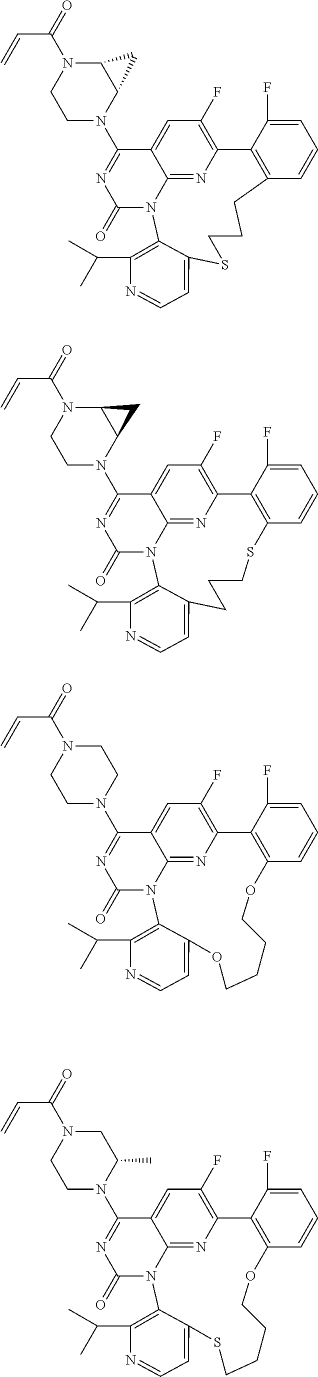 [SMILES: C=CC(=O)N1CCN(c2nc(=O)n3c4nc(c(F)cc24)-c2c(F)cccc2CCCSc2ccnc(C(C)C)c2-3)[C@H]2C[C@H]21 ; C=CC(=O)N1CCN(c2nc(=O)n3c4nc(c(F)cc24)-c2c(F)cccc2SCCCc2ccnc(C(C)C)c2-3)[C@@H]2C[C@@H]21 ; C=CC(=O)N1CCN(c2nc(=O)n3c4nc(c(F)cc24)-c2c(F)cccc2OCCCCOc2ccnc(C(C)C)c2-3)CC1 ; C=CC(=O)N1CCN(c2nc(=O)n3c4nc(c(F)cc24)-c2c(F)cccc2OCCCCSc2ccnc(C(C)C)c2-3)[C@@H](C)C1]

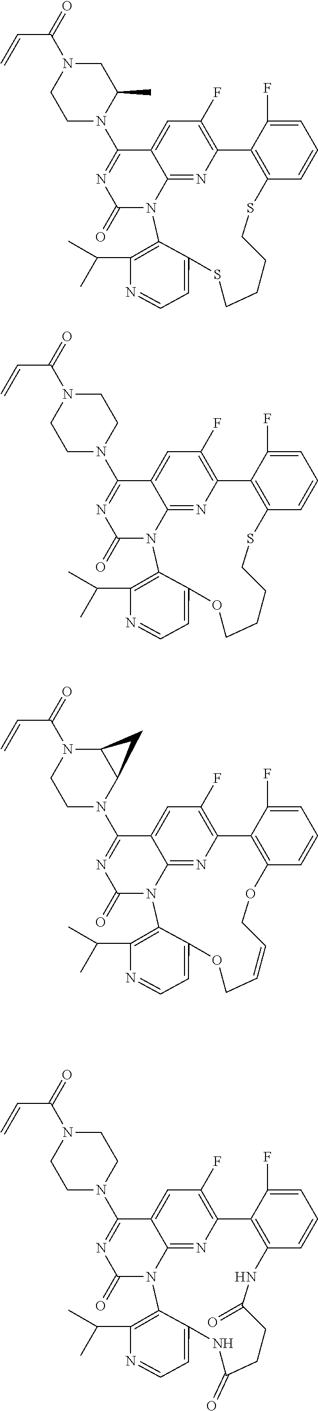 [SMILES: C=CC(=O)N1CCN(c2nc(=O)n3c4nc(c(F)cc24)-c2c(F)cccc2SCCCCSc2ccnc(C(C)C)c2-3)[C@H](C)C1 ; C=CC(=O)N1CCN(c2nc(=O)n3c4nc(c(F)cc24)-c2c(F)cccc2SCCCCOc2ccnc(C(C)C)c2-3)CC1 ; C=CC(=O)N1CCN(c2nc(=O)n3c4nc(c(F)cc24)-c2c(F)cccc2OC/C=C\COc2ccnc(C(C)C)c2-3)[C@@H]2C[C@@H]21 ; C=CC(=O)N1CCN(c2nc(=O)n3c4nc(c(F)cc24)-c2c(F)cccc2NC(=O)CCC(=O)Nc2ccnc(C(C)C)c2-3)CC1]

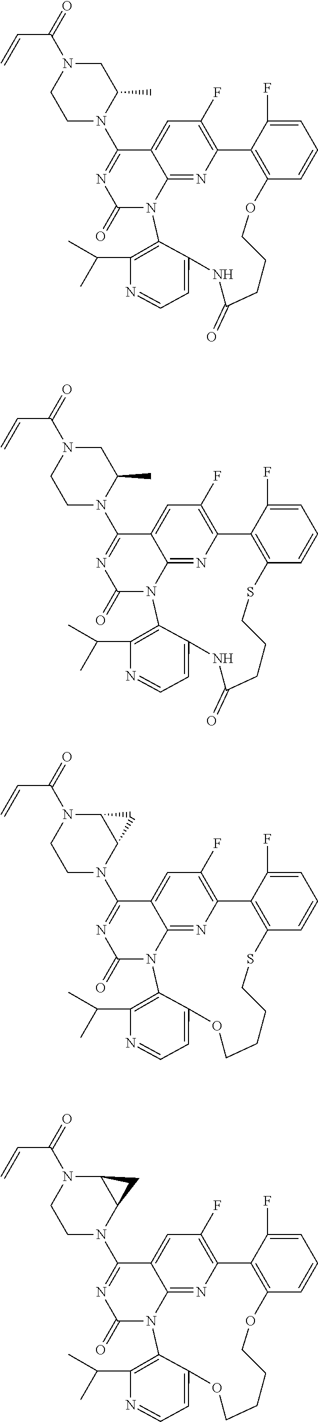 [SMILES: C=CC(=O)N1CCN(c2nc(=O)n3c4nc(c(F)cc24)-c2c(F)cccc2OCCCC(=O)Nc2ccnc(C(C)C)c2-3)[C@@H](C)C1 ; C=CC(=O)N1CCN(c2nc(=O)n3c4nc(c(F)cc24)-c2c(F)cccc2SCCCC(=O)Nc2ccnc(C(C)C)c2-3)[C@H](C)C1 ; C=CC(=O)N1CCN(c2nc(=O)n3c4nc(c(F)cc24)-c2c(F)cccc2SCCCCOc2ccnc(C(C)C)c2-3)[C@H]2C[C@H]21 ; C=CC(=O)N1CCN(c2nc(=O)n3c4nc(c(F)cc24)-c2c(F)cccc2OCCCCOc2ccnc(C(C)C)c2-3)[C@@H]2C[C@@H]21]

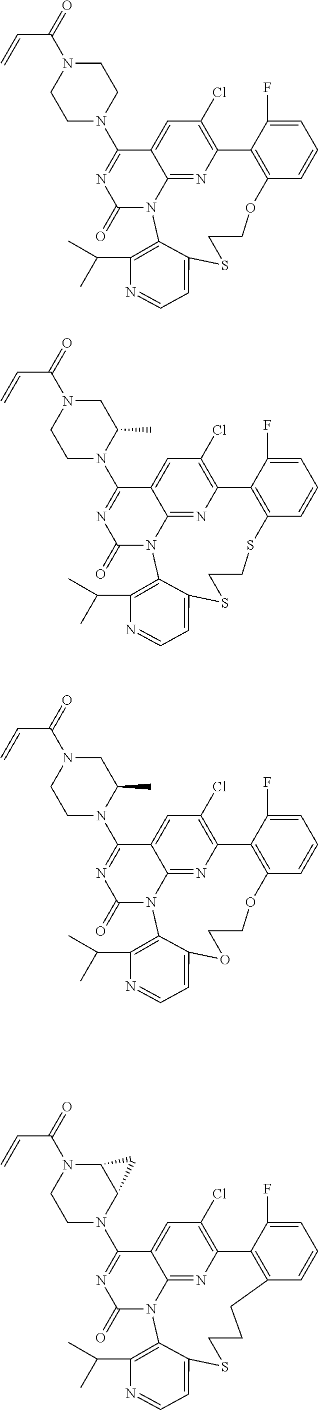 [SMILES: C=CC(=O)N1CCN(c2nc(=O)n3c4nc(c(Cl)cc24)-c2c(F)cccc2OCCSc2ccnc(C(C)C)c2-3)CC1 ; C=CC(=O)N1CCN(c2nc(=O)n3c4nc(c(Cl)cc24)-c2c(F)cccc2SCCSc2ccnc(C(C)C)c2-3)[C@@H](C)C1 ; C=CC(=O)N1CCN(c2nc(=O)n3c4nc(c(Cl)cc24)-c2c(F)cccc2OCCOc2ccnc(C(C)C)c2-3)[C@H](C)C1 ; C=CC(=O)N1CCN(c2nc(=O)n3c4nc(c(Cl)cc24)-c2c(F)cccc2CCCSc2ccnc(C(C)C)c2-3)[C@H]2C[C@H]21]

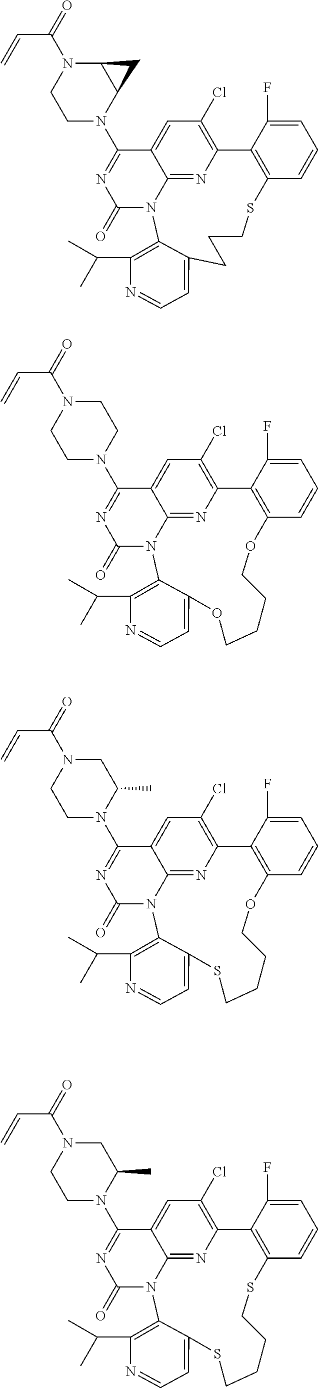 [SMILES: C=CC(=O)N1CCN(c2nc(=O)n3c4nc(c(Cl)cc24)-c2c(F)cccc2SCCCc2ccnc(C(C)C)c2-3)[C@@H]2C[C@@H]21 ; C=CC(=O)N1CCN(c2nc(=O)n3c4nc(c(Cl)cc24)-c2c(F)cccc2OCCCCOc2ccnc(C(C)C)c2-3)CC1 ; C=CC(=O)N1CCN(c2nc(=O)n3c4nc(c(Cl)cc24)-c2c(F)cccc2OCCCCSc2ccnc(C(C)C)c2-3)[C@@H](C)C1 ; C=CC(=O)N1CCN(c2nc(=O)n3c4nc(c(Cl)cc24)-c2c(F)cccc2SCCCCSc2ccnc(C(C)C)c2-3)[C@H](C)C1]

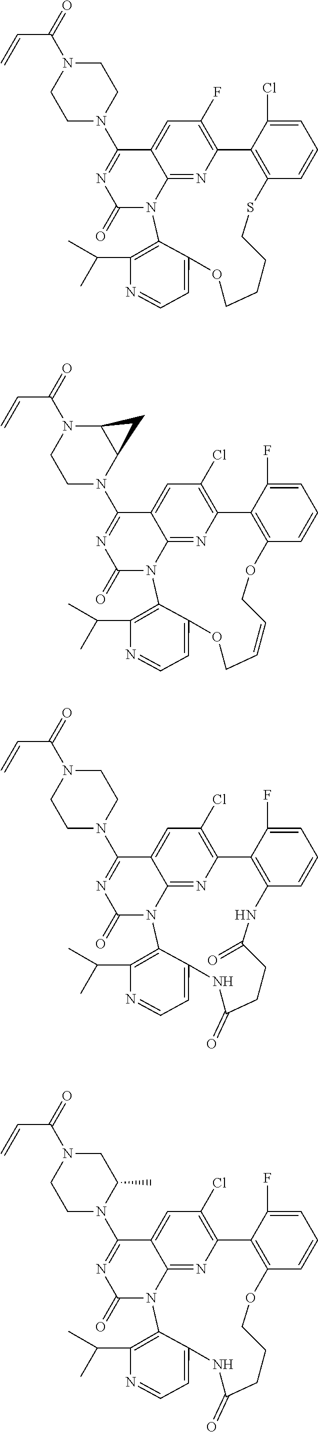 [SMILES: C=CC(=O)N1CCN(c2nc(=O)n3c4nc(c(F)cc24)-c2c(Cl)cccc2SCCCCOc2ccnc(C(C)C)c2-3)CC1 ; C=CC(=O)N1CCN(c2nc(=O)n3c4nc(c(Cl)cc24)-c2c(F)cccc2OC/C=C\COc2ccnc(C(C)C)c2-3)[C@@H]2C[C@@H]21 ; C=CC(=O)N1CCN(c2nc(=O)n3c4nc(c(Cl)cc24)-c2c(F)cccc2NC(=O)CCC(=O)Nc2ccnc(C(C)C)c2-3)CC1 ; C=CC(=O)N1CCN(c2nc(=O)n3c4nc(c(Cl)cc24)-c2c(F)cccc2OCCCC(=O)Nc2ccnc(C(C)C)c2-3)[C@@H](C)C1]

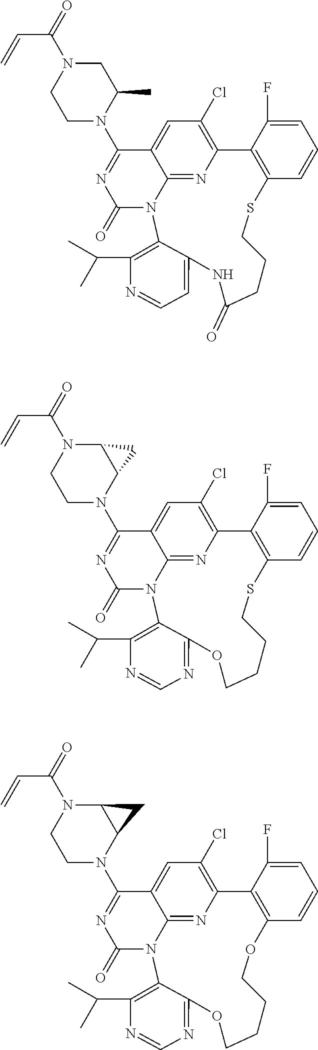 [SMILES: C=CC(=O)N1CCN(c2nc(=O)n3c4nc(c(Cl)cc24)-c2c(F)cccc2SCCCC(=O)Nc2ccnc(C(C)C)c2-3)[C@H](C)C1 ; C=CC(=O)N1CCN(c2nc(=O)n3c4nc(c(Cl)cc24)-c2c(F)cccc2SCCCCOc2ncnc(C(C)C)c2-3)[C@H]2C[C@H]21 ; C=CC(=O)N1CCN(c2nc(=O)n3c4nc(c(Cl)cc24)-c2c(F)cccc2OCCCCOc2ncnc(C(C)C)c2-3)[C@@H]2C[C@@H]21]

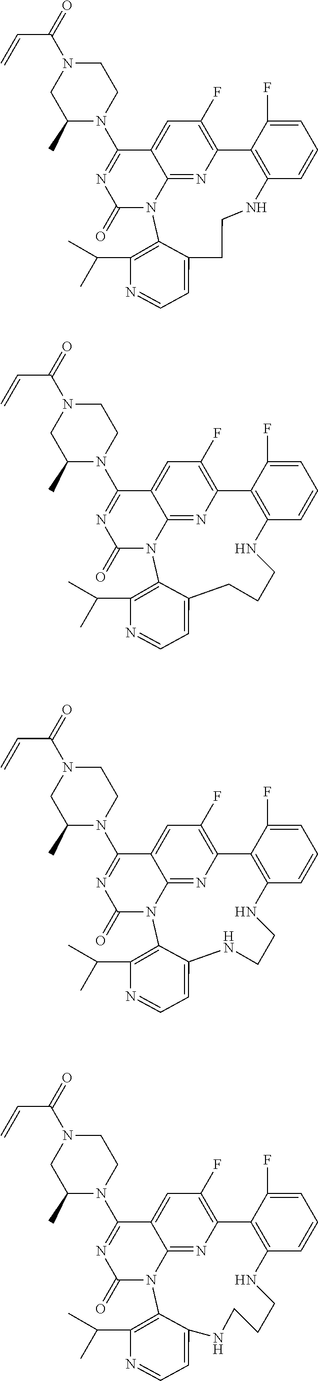 [SMILES: C=CC(=O)N1CCN(c2nc(=O)n3c4nc(c(F)cc24)-c2c(F)cccc2NCCc2ccnc(C(C)C)c2-3)[C@@H](C)C1 ; C=CC(=O)N1CCN(c2nc(=O)n3c4nc(c(F)cc24)-c2c(F)cccc2NCCCc2ccnc(C(C)C)c2-3)[C@@H](C)C1 ; C=CC(=O)N1CCN(c2nc(=O)n3c4nc(c(F)cc24)-c2c(F)cccc2NCCNc2ccnc(C(C)C)c2-3)[C@@H](C)C1 ; C=CC(=O)N1CCN(c2nc(=O)n3c4nc(c(F)cc24)-c2c(F)cccc2NCCCNc2ccnc(C(C)C)c2-3)[C@@H](C)C1]

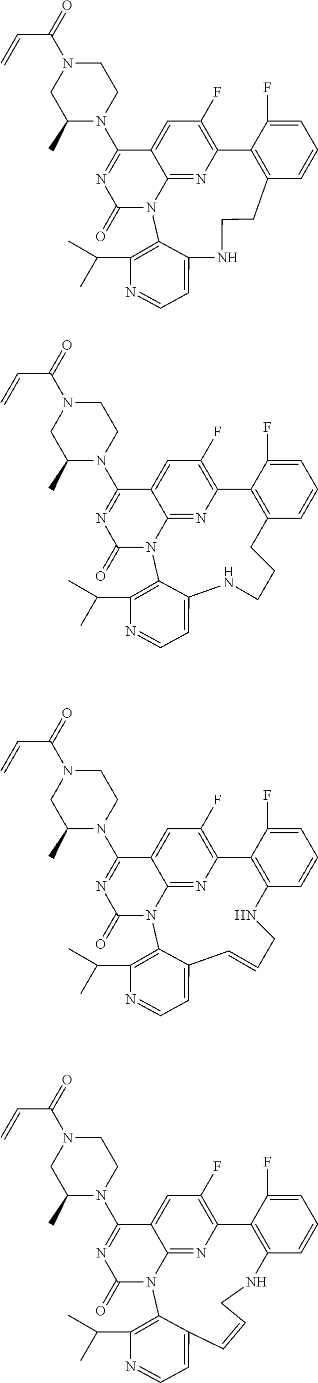 [SMILES: C=CC(=O)N1CCN(c2nc(=O)n3c4nc(c(F)cc24)-c2c(F)cccc2CCNc2ccnc(C(C)C)c2-3)[C@@H](C)C1 ; C=CC(=O)N1CCN(c2nc(=O)n3c4nc(c(F)cc24)-c2c(F)cccc2CCCNc2ccnc(C(C)C)c2-3)[C@@H](C)C1 ; C=CC(=O)N1CCN(c2nc(=O)n3c4nc(c(F)cc24)-c2c(F)cccc2NC/C=C/c2ccnc(C(C)C)c2-3)[C@@H](C)C1 ; C=CC(=O)N1CCN(c2nc(=O)n3c4nc(c(F)cc24)-c2c(F)cccc2NC/C=C\c2ccnc(C(C)C)c2-3)[C@@H](C)C1]

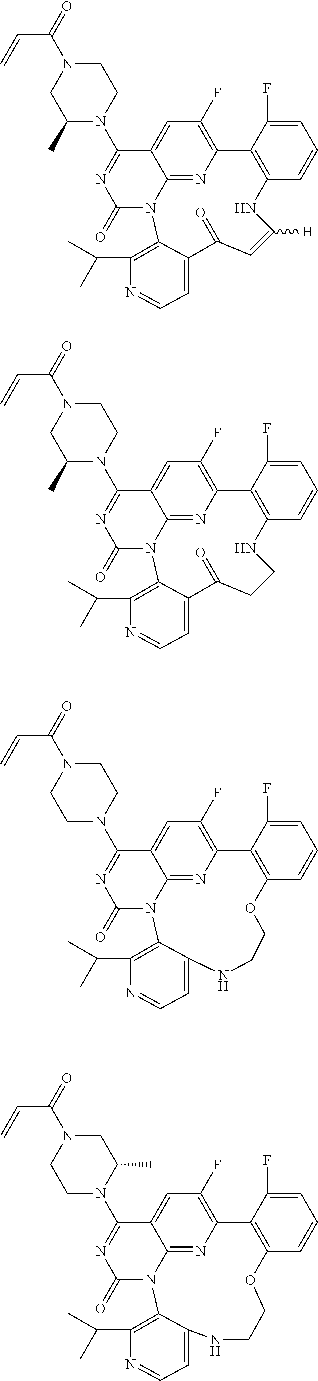 [SMILES: C=CC(=O)N1CCN(c2nc(=O)n3c4nc(c(F)cc24)-c2c(F)cccc2NC=CC(=O)c2ccnc(C(C)C)c2-3)[C@@H](C)C1 ; C=CC(=O)N1CCN(c2nc(=O)n3c4nc(c(F)cc24)-c2c(F)cccc2NCCC(=O)c2ccnc(C(C)C)c2-3)[C@@H](C)C1 ; C=CC(=O)N1CCN(c2nc(=O)n3c4nc(c(F)cc24)-c2c(F)cccc2OCCNc2ccnc(C(C)C)c2-3)CC1 ; C=CC(=O)N1CCN(c2nc(=O)n3c4nc(c(F)cc24)-c2c(F)cccc2OCCNc2ccnc(C(C)C)c2-3)[C@@H](C)C1]

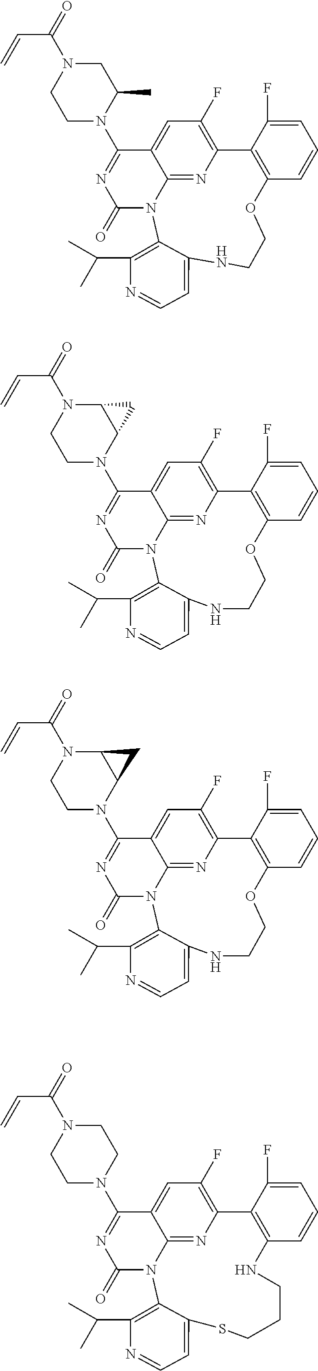 [SMILES: C=CC(=O)N1CCN(c2nc(=O)n3c4nc(c(F)cc24)-c2c(F)cccc2OCCNc2ccnc(C(C)C)c2-3)[C@H](C)C1 ; C=CC(=O)N1CCN(c2nc(=O)n3c4nc(c(F)cc24)-c2c(F)cccc2OCCNc2ccnc(C(C)C)c2-3)[C@H]2C[C@H]21 ; C=CC(=O)N1CCN(c2nc(=O)n3c4nc(c(F)cc24)-c2c(F)cccc2OCCNc2ccnc(C(C)C)c2-3)[C@@H]2C[C@@H]21 ; C=CC(=O)N1CCN(c2nc(=O)n3c4nc(c(F)cc24)-c2c(F)cccc2NCCCSc2ccnc(C(C)C)c2-3)CC1]

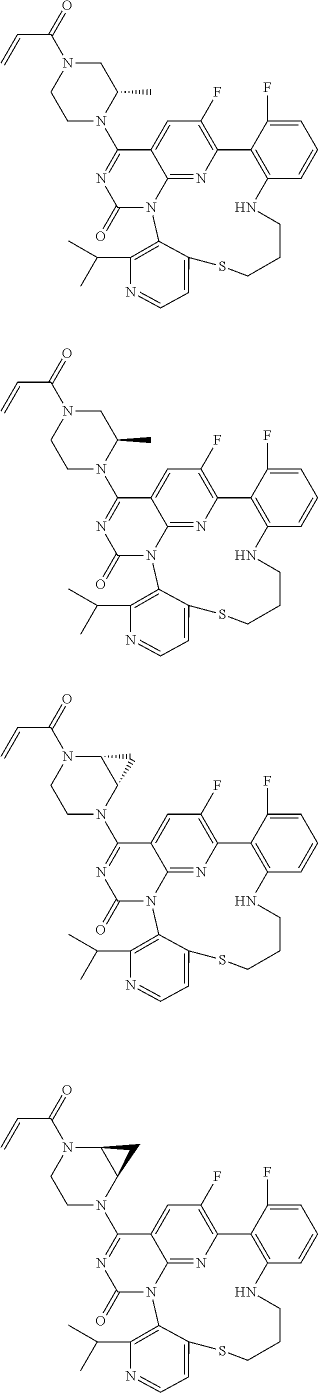 [SMILES: C=CC(=O)N1CCN(c2nc(=O)n3c4nc(c(F)cc24)-c2c(F)cccc2NCCCSc2ccnc(C(C)C)c2-3)[C@@H](C)C1 ; C=CC(=O)N1CCN(c2nc(=O)n3c4nc(c(F)cc24)-c2c(F)cccc2NCCCSc2ccnc(C(C)C)c2-3)[C@H](C)C1 ; C=CC(=O)N1CCN(c2nc(=O)n3c4nc(c(F)cc24)-c2c(F)cccc2NCCCSc2ccnc(C(C)C)c2-3)[C@H]2C[C@H]21 ; C=CC(=O)N1CCN(c2nc(=O)n3c4nc(c(F)cc24)-c2c(F)cccc2NCCCSc2ccnc(C(C)C)c2-3)[C@@H]2C[C@@H]21]

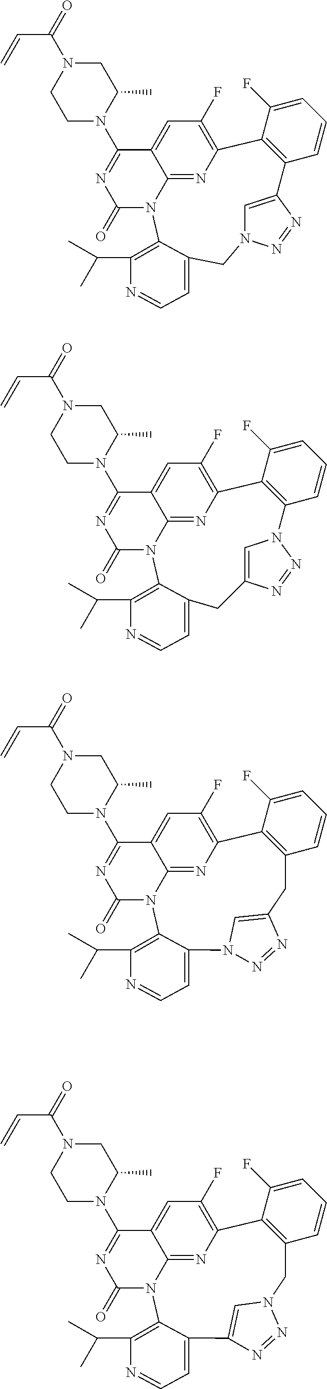 [SMILES: C=CC(=O)N1CCN(c2nc(=O)n3c4nc(c(F)cc24)-c2c(F)cccc2-c2cn(nn2)Cc2ccnc(C(C)C)c2-3)[C@@H](C)C1 ; C=CC(=O)N1CCN(c2nc(=O)n3c4nc(c(F)cc24)-c2c(F)cccc2-n2cc(nn2)Cc2ccnc(C(C)C)c2-3)[C@@H](C)C1 ; C=CC(=O)N1CCN(c2nc(=O)n3c4nc(c(F)cc24)-c2c(F)cccc2Cc2cn(nn2)-c2ccnc(C(C)C)c2-3)[C@@H](C)C1 ; C=CC(=O)N1CCN(c2nc(=O)n3c4nc(c(F)cc24)-c2c(F)cccc2Cn2cc(nn2)-c2ccnc(C(C)C)c2-3)[C@@H](C)C1]

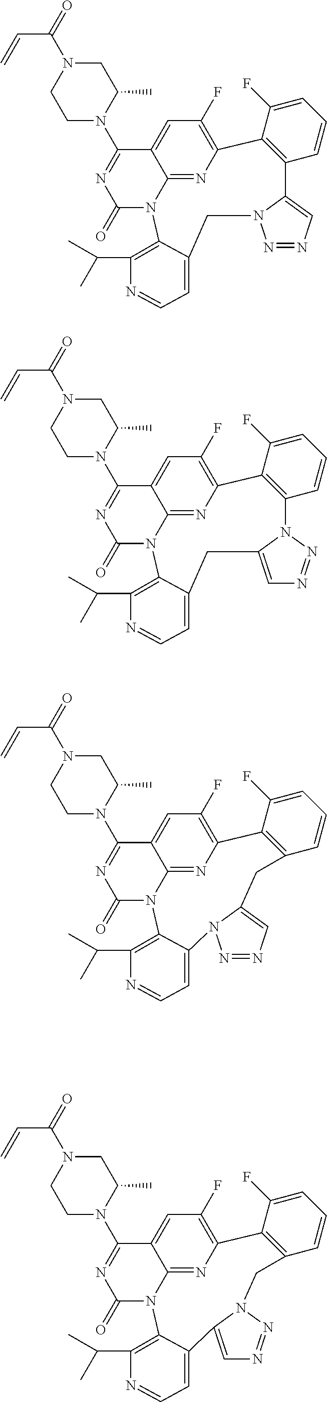 [SMILES: C=CC(=O)N1CCN(c2nc(=O)n3c4nc(c(F)cc24)-c2c(F)cccc2-c2cnnn2Cc2ccnc(C(C)C)c2-3)[C@@H](C)C1 ; C=CC(=O)N1CCN(c2nc(=O)n3c4nc(c(F)cc24)-c2c(F)cccc2-n2nncc2Cc2ccnc(C(C)C)c2-3)[C@@H](C)C1 ; C=CC(=O)N1CCN(c2nc(=O)n3c4nc(c(F)cc24)-c2c(F)cccc2Cc2cnnn2-c2ccnc(C(C)C)c2-3)[C@@H](C)C1 ; C=CC(=O)N1CCN(c2nc(=O)n3c4nc(c(F)cc24)-c2c(F)cccc2Cn2nncc2-c2ccnc(C(C)C)c2-3)[C@@H](C)C1]

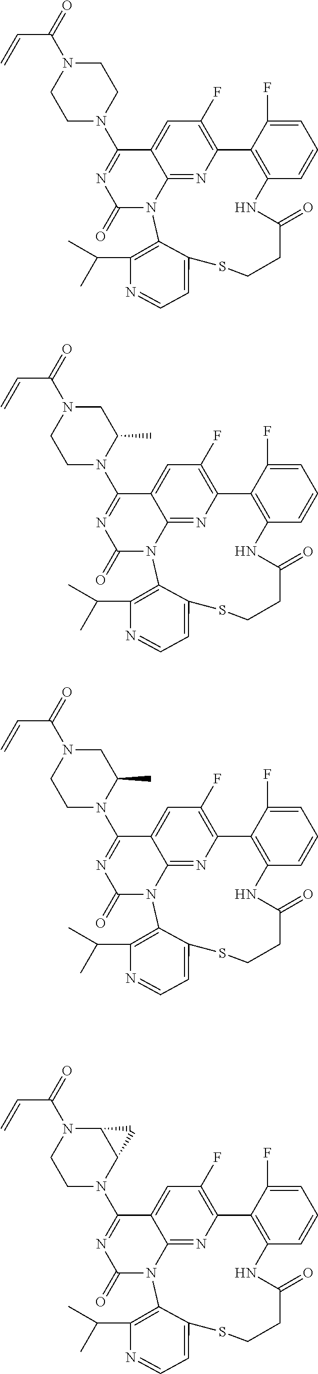 [SMILES: C=CC(=O)N1CCN(c2nc(=O)n3c4nc(c(F)cc24)-c2c(F)cccc2NC(=O)CCSc2ccnc(C(C)C)c2-3)CC1 ; C=CC(=O)N1CCN(c2nc(=O)n3c4nc(c(F)cc24)-c2c(F)cccc2NC(=O)CCSc2ccnc(C(C)C)c2-3)[C@@H](C)C1 ; C=CC(=O)N1CCN(c2nc(=O)n3c4nc(c(F)cc24)-c2c(F)cccc2NC(=O)CCSc2ccnc(C(C)C)c2-3)[C@H](C)C1 ; C=CC(=O)N1CCN(c2nc(=O)n3c4nc(c(F)cc24)-c2c(F)cccc2NC(=O)CCSc2ccnc(C(C)C)c2-3)[C@H]2C[C@H]21]

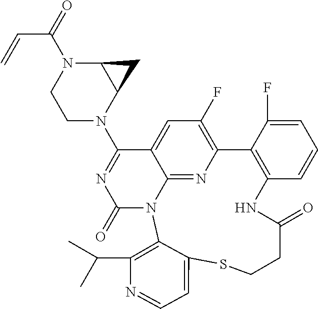 [SMILES: C=CC(=O)N1CCN(c2nc(=O)n3c4nc(c(F)cc24)-c2c(F)cccc2NC(=O)CCSc2ccnc(C(C)C)c2-3)[C@@H]2C[C@@H]21]

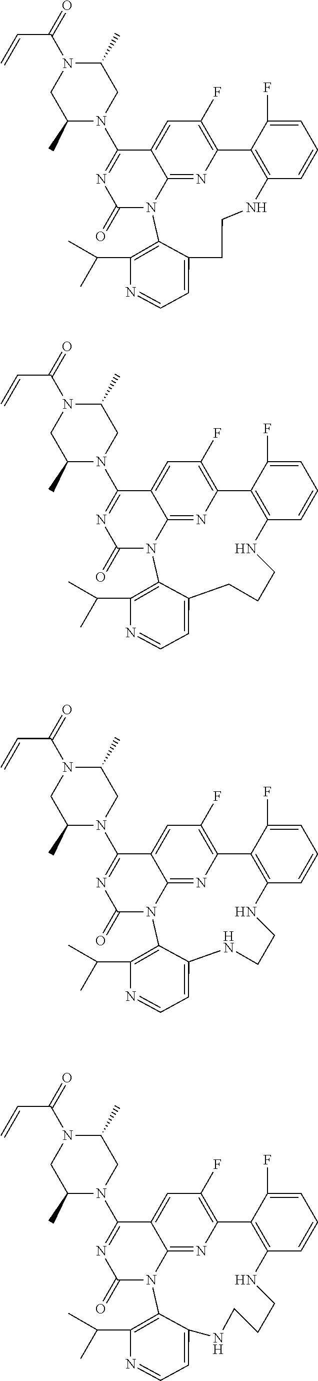 [SMILES: C=CC(=O)N1C[C@H](C)N(c2nc(=O)n3c4nc(c(F)cc24)-c2c(F)cccc2NCCc2ccnc(C(C)C)c2-3)C[C@H]1C ; C=CC(=O)N1C[C@H](C)N(c2nc(=O)n3c4nc(c(F)cc24)-c2c(F)cccc2NCCCc2ccnc(C(C)C)c2-3)C[C@H]1C ; C=CC(=O)N1C[C@H](C)N(c2nc(=O)n3c4nc(c(F)cc24)-c2c(F)cccc2NCCNc2ccnc(C(C)C)c2-3)C[C@H]1C ; C=CC(=O)N1C[C@H](C)N(c2nc(=O)n3c4nc(c(F)cc24)-c2c(F)cccc2NCCCNc2ccnc(C(C)C)c2-3)C[C@H]1C]

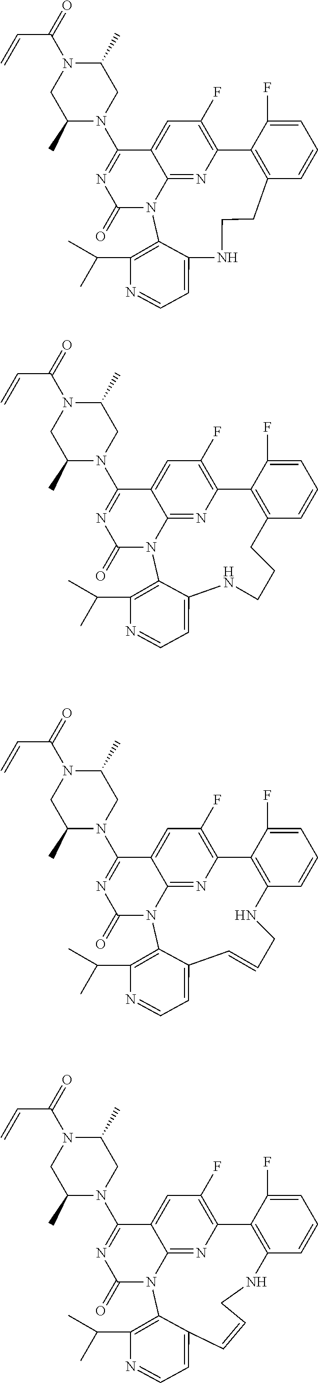 [SMILES: C=CC(=O)N1C[C@H](C)N(c2nc(=O)n3c4nc(c(F)cc24)-c2c(F)cccc2CCNc2ccnc(C(C)C)c2-3)C[C@H]1C ; C=CC(=O)N1C[C@H](C)N(c2nc(=O)n3c4nc(c(F)cc24)-c2c(F)cccc2CCCNc2ccnc(C(C)C)c2-3)C[C@H]1C ; C=CC(=O)N1C[C@H](C)N(c2nc(=O)n3c4nc(c(F)cc24)-c2c(F)cccc2NC/C=C/c2ccnc(C(C)C)c2-3)C[C@H]1C ; C=CC(=O)N1C[C@H](C)N(c2nc(=O)n3c4nc(c(F)cc24)-c2c(F)cccc2NC/C=C\c2ccnc(C(C)C)c2-3)C[C@H]1C]

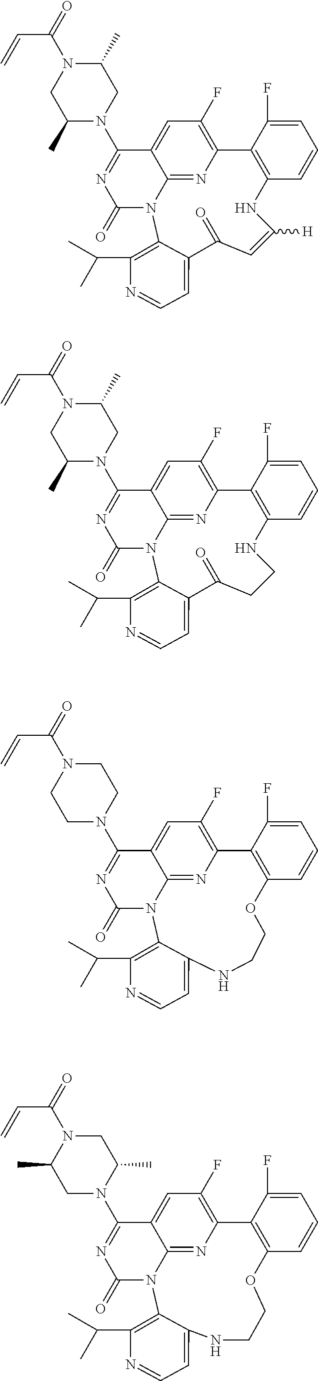 [SMILES: C=CC(=O)N1C[C@H](C)N(c2nc(=O)n3c4nc(c(F)cc24)-c2c(F)cccc2NC=CC(=O)c2ccnc(C(C)C)c2-3)C[C@H]1C ; C=CC(=O)N1C[C@H](C)N(c2nc(=O)n3c4nc(c(F)cc24)-c2c(F)cccc2NCCC(=O)c2ccnc(C(C)C)c2-3)C[C@H]1C ; C=CC(=O)N1CCN(c2nc(=O)n3c4nc(c(F)cc24)-c2c(F)cccc2OCCNc2ccnc(C(C)C)c2-3)CC1 ; C=CC(=O)N1C[C@H](C)N(c2nc(=O)n3c4nc(c(F)cc24)-c2c(F)cccc2OCCNc2ccnc(C(C)C)c2-3)C[C@H]1C]

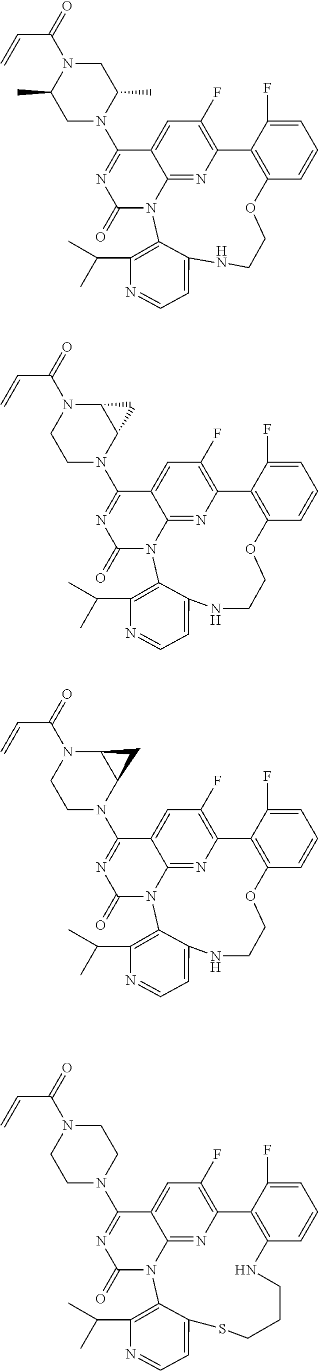 [SMILES: C=CC(=O)N1C[C@H](C)N(c2nc(=O)n3c4nc(c(F)cc24)-c2c(F)cccc2OCCNc2ccnc(C(C)C)c2-3)C[C@H]1C ; C=CC(=O)N1CCN(c2nc(=O)n3c4nc(c(F)cc24)-c2c(F)cccc2OCCNc2ccnc(C(C)C)c2-3)[C@H]2C[C@H]21 ; C=CC(=O)N1CCN(c2nc(=O)n3c4nc(c(F)cc24)-c2c(F)cccc2OCCNc2ccnc(C(C)C)c2-3)[C@@H]2C[C@@H]21 ; C=CC(=O)N1CCN(c2nc(=O)n3c4nc(c(F)cc24)-c2c(F)cccc2NCCCSc2ccnc(C(C)C)c2-3)CC1]

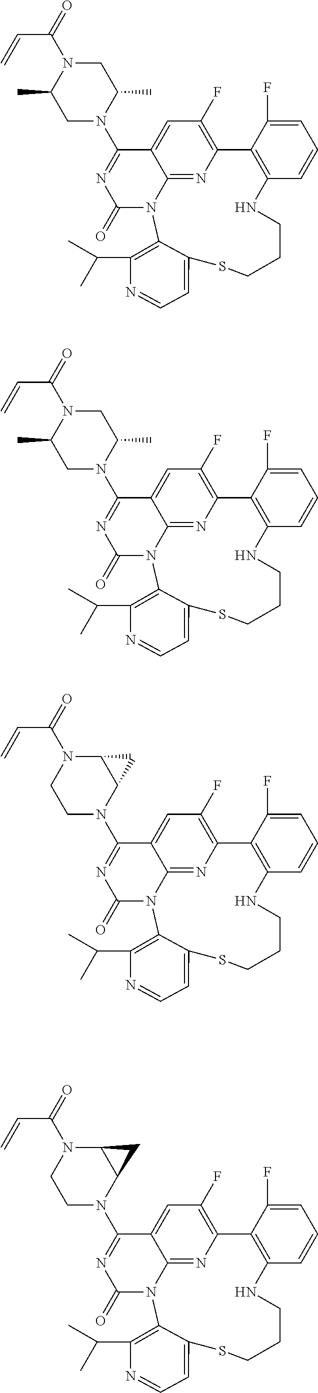 [SMILES: C=CC(=O)N1C[C@H](C)N(c2nc(=O)n3c4nc(c(F)cc24)-c2c(F)cccc2NCCCSc2ccnc(C(C)C)c2-3)C[C@H]1C ; C=CC(=O)N1C[C@H](C)N(c2nc(=O)n3c4nc(c(F)cc24)-c2c(F)cccc2NCCCSc2ccnc(C(C)C)c2-3)C[C@H]1C ; C=CC(=O)N1CCN(c2nc(=O)n3c4nc(c(F)cc24)-c2c(F)cccc2NCCCSc2ccnc(C(C)C)c2-3)[C@H]2C[C@H]21 ; C=CC(=O)N1CCN(c2nc(=O)n3c4nc(c(F)cc24)-c2c(F)cccc2NCCCSc2ccnc(C(C)C)c2-3)[C@@H]2C[C@@H]21]

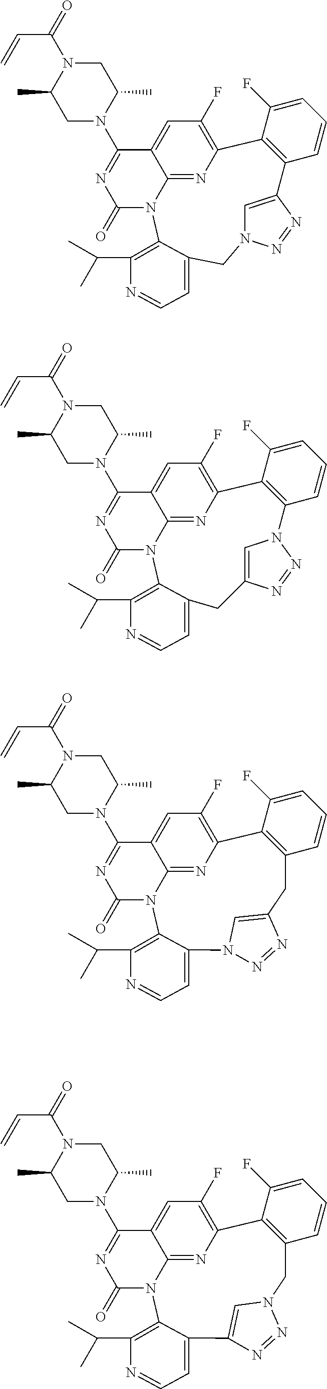 [SMILES: C=CC(=O)N1C[C@H](C)N(c2nc(=O)n3c4nc(c(F)cc24)-c2c(F)cccc2-c2cn(nn2)Cc2ccnc(C(C)C)c2-3)C[C@H]1C ; C=CC(=O)N1C[C@H](C)N(c2nc(=O)n3c4nc(c(F)cc24)-c2c(F)cccc2-n2cc(nn2)Cc2ccnc(C(C)C)c2-3)C[C@H]1C ; C=CC(=O)N1C[C@H](C)N(c2nc(=O)n3c4nc(c(F)cc24)-c2c(F)cccc2Cc2cn(nn2)-c2ccnc(C(C)C)c2-3)C[C@H]1C ; C=CC(=O)N1C[C@H](C)N(c2nc(=O)n3c4nc(c(F)cc24)-c2c(F)cccc2Cn2cc(nn2)-c2ccnc(C(C)C)c2-3)C[C@H]1C]

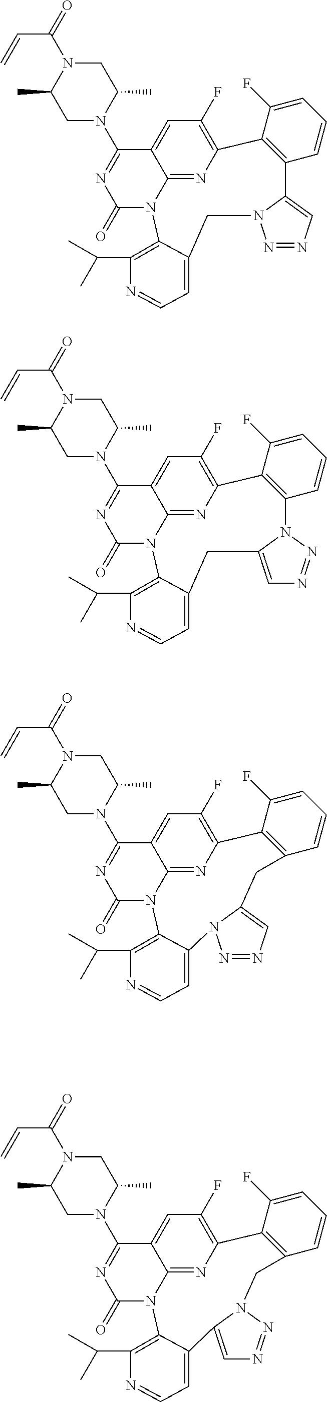 [SMILES: C=CC(=O)N1C[C@H](C)N(c2nc(=O)n3c4nc(c(F)cc24)-c2c(F)cccc2-c2cnnn2Cc2ccnc(C(C)C)c2-3)C[C@H]1C ; C=CC(=O)N1C[C@H](C)N(c2nc(=O)n3c4nc(c(F)cc24)-c2c(F)cccc2-n2nncc2Cc2ccnc(C(C)C)c2-3)C[C@H]1C ; C=CC(=O)N1C[C@H](C)N(c2nc(=O)n3c4nc(c(F)cc24)-c2c(F)cccc2Cc2cnnn2-c2ccnc(C(C)C)c2-3)C[C@H]1C ; C=CC(=O)N1C[C@H](C)N(c2nc(=O)n3c4nc(c(F)cc24)-c2c(F)cccc2Cn2nncc2-c2ccnc(C(C)C)c2-3)C[C@H]1C]

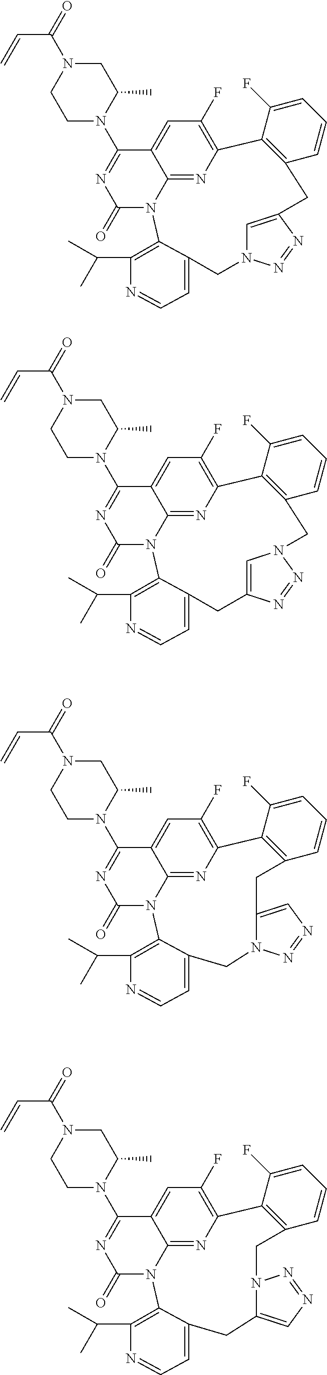 [SMILES: C=CC(=O)N1CCN(c2nc(=O)n3c4nc(c(F)cc24)-c2c(F)cccc2Cc2cn(nn2)Cc2ccnc(C(C)C)c2-3)[C@@H](C)C1 ; C=CC(=O)N1CCN(c2nc(=O)n3c4nc(c(F)cc24)-c2c(F)cccc2Cn2cc(nn2)Cc2ccnc(C(C)C)c2-3)[C@@H](C)C1 ; C=CC(=O)N1CCN(c2nc(=O)n3c4nc(c(F)cc24)-c2c(F)cccc2Cc2cnnn2Cc2ccnc(C(C)C)c2-3)[C@@H](C)C1 ; C=CC(=O)N1CCN(c2nc(=O)n3c4nc(c(F)cc24)-c2c(F)cccc2Cn2nncc2Cc2ccnc(C(C)C)c2-3)[C@@H](C)C1]

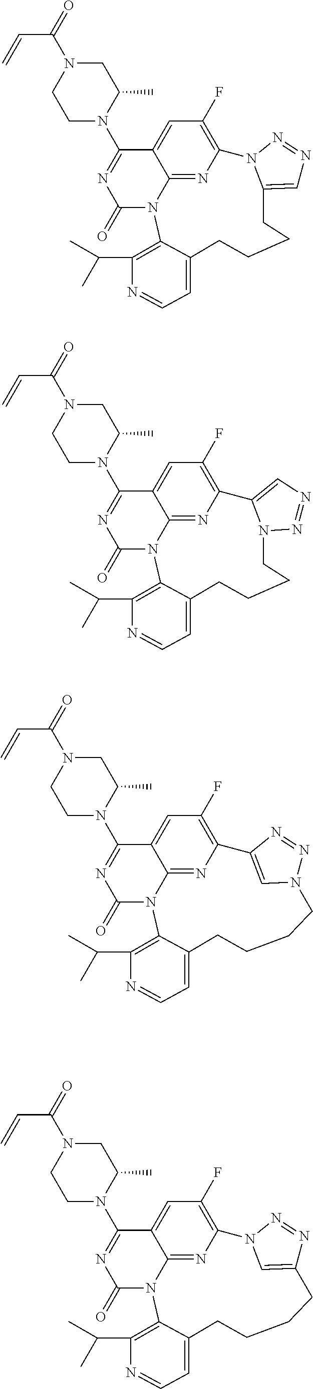 [SMILES: C=CC(=O)N1CCN(c2nc(=O)n3c4nc(c(F)cc24)-n2nncc2CCCCc2ccnc(C(C)C)c2-3)[C@@H](C)C1 ; C=CC(=O)N1CCN(c2nc(=O)n3c4nc(c(F)cc24)-c2cnnn2CCCCc2ccnc(C(C)C)c2-3)[C@@H](C)C1 ; C=CC(=O)N1CCN(c2nc(=O)n3c4nc(c(F)cc24)-c2cn(nn2)CCCCc2ccnc(C(C)C)c2-3)[C@@H](C)C1 ; C=CC(=O)N1CCN(c2nc(=O)n3c4nc(c(F)cc24)-n2cc(nn2)CCCCc2ccnc(C(C)C)c2-3)[C@@H](C)C1]

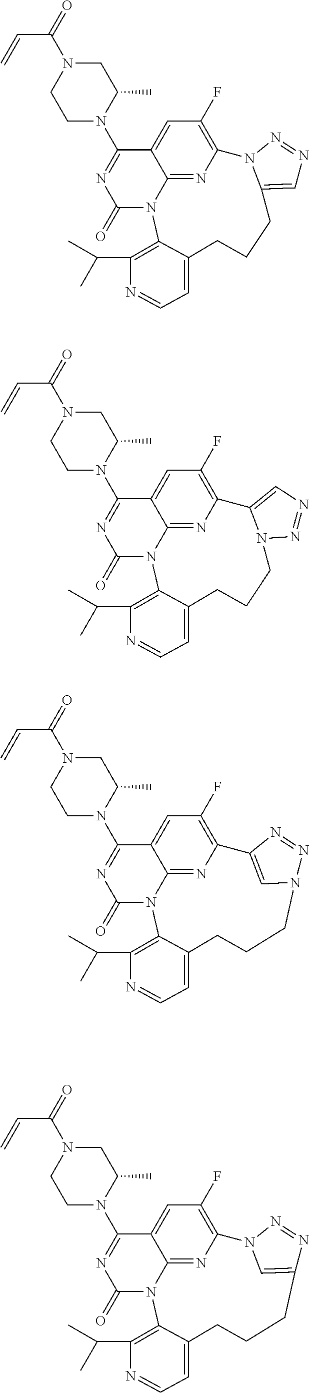 [SMILES: C=CC(=O)N1CCN(c2nc(=O)n3c4nc(c(F)cc24)-n2nncc2CCCc2ccnc(C(C)C)c2-3)[C@@H](C)C1 ; C=CC(=O)N1CCN(c2nc(=O)n3c4nc(c(F)cc24)-c2cnnn2CCCc2ccnc(C(C)C)c2-3)[C@@H](C)C1 ; C=CC(=O)N1CCN(c2nc(=O)n3c4nc(c(F)cc24)-c2cn(nn2)CCCc2ccnc(C(C)C)c2-3)[C@@H](C)C1 ; C=CC(=O)N1CCN(c2nc(=O)n3c4nc(c(F)cc24)-n2cc(nn2)CCCc2ccnc(C(C)C)c2-3)[C@@H](C)C1]

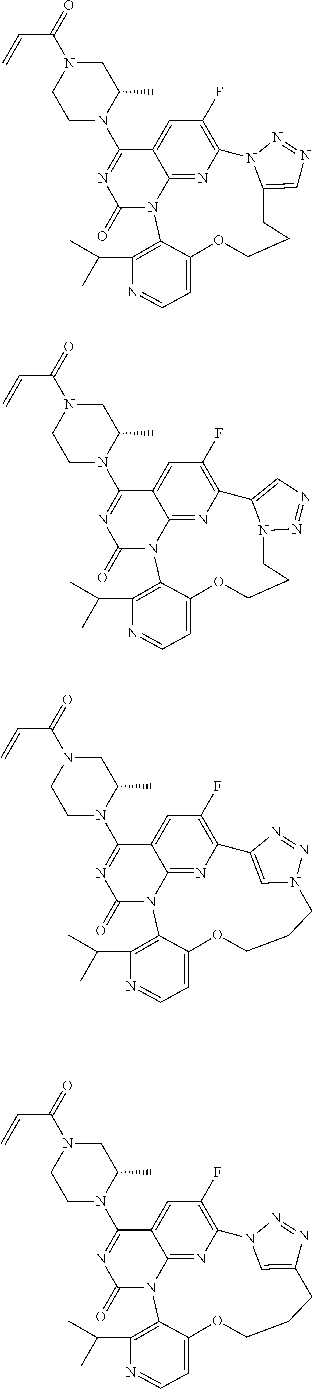 [SMILES: C=CC(=O)N1CCN(c2nc(=O)n3c4nc(c(F)cc24)-n2nncc2CCCOc2ccnc(C(C)C)c2-3)[C@@H](C)C1 ; C=CC(=O)N1CCN(c2nc(=O)n3c4nc(c(F)cc24)-c2cnnn2CCCOc2ccnc(C(C)C)c2-3)[C@@H](C)C1 ; C=CC(=O)N1CCN(c2nc(=O)n3c4nc(c(F)cc24)-c2cn(nn2)CCCOc2ccnc(C(C)C)c2-3)[C@@H](C)C1 ; C=CC(=O)N1CCN(c2nc(=O)n3c4nc(c(F)cc24)-n2cc(nn2)CCCOc2ccnc(C(C)C)c2-3)[C@@H](C)C1]

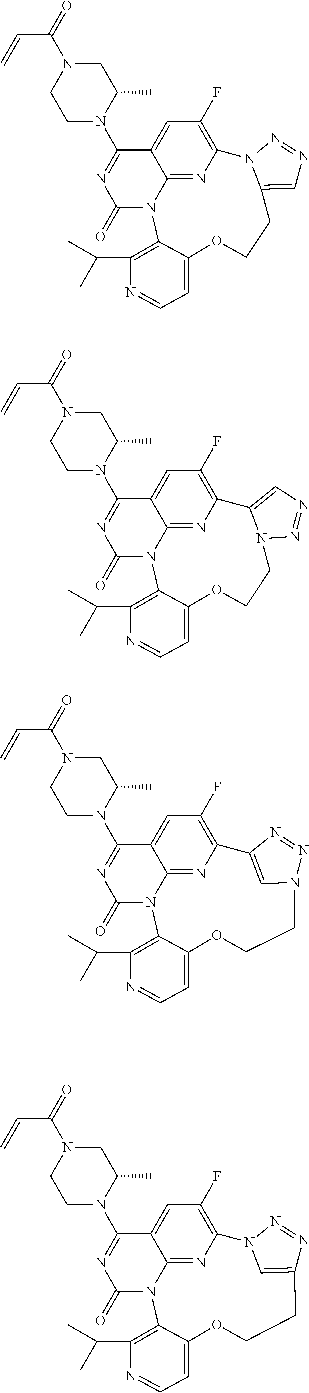 [SMILES: C=CC(=O)N1CCN(c2nc(=O)n3c4nc(c(F)cc24)-n2nncc2CCOc2ccnc(C(C)C)c2-3)[C@@H](C)C1 ; C=CC(=O)N1CCN(c2nc(=O)n3c4nc(c(F)cc24)-c2cnnn2CCOc2ccnc(C(C)C)c2-3)[C@@H](C)C1 ; C=CC(=O)N1CCN(c2nc(=O)n3c4nc(c(F)cc24)-c2cn(nn2)CCOc2ccnc(C(C)C)c2-3)[C@@H](C)C1 ; C=CC(=O)N1CCN(c2nc(=O)n3c4nc(c(F)cc24)-n2cc(nn2)CCOc2ccnc(C(C)C)c2-3)[C@@H](C)C1]

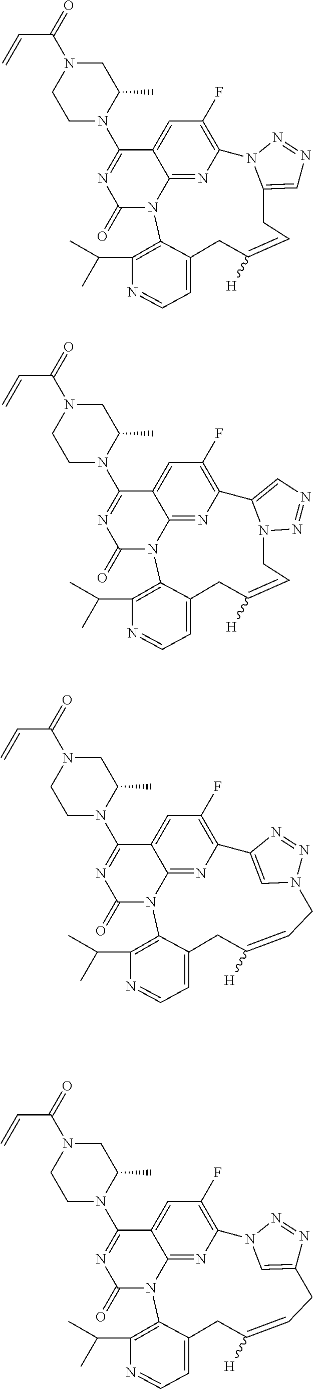 [SMILES: C=CC(=O)N1CCN(c2nc(=O)n3c4nc(c(F)cc24)-n2nncc2CC=CCc2ccnc(C(C)C)c2-3)[C@@H](C)C1 ; C=CC(=O)N1CCN(c2nc(=O)n3c4nc(c(F)cc24)-c2cnnn2CC=CCc2ccnc(C(C)C)c2-3)[C@@H](C)C1 ; C=CC(=O)N1CCN(c2nc(=O)n3c4nc(c(F)cc24)-c2cn(nn2)CC=CCc2ccnc(C(C)C)c2-3)[C@@H](C)C1 ; C=CC(=O)N1CCN(c2nc(=O)n3c4nc(c(F)cc24)-n2cc(nn2)CC=CCc2ccnc(C(C)C)c2-3)[C@@H](C)C1]

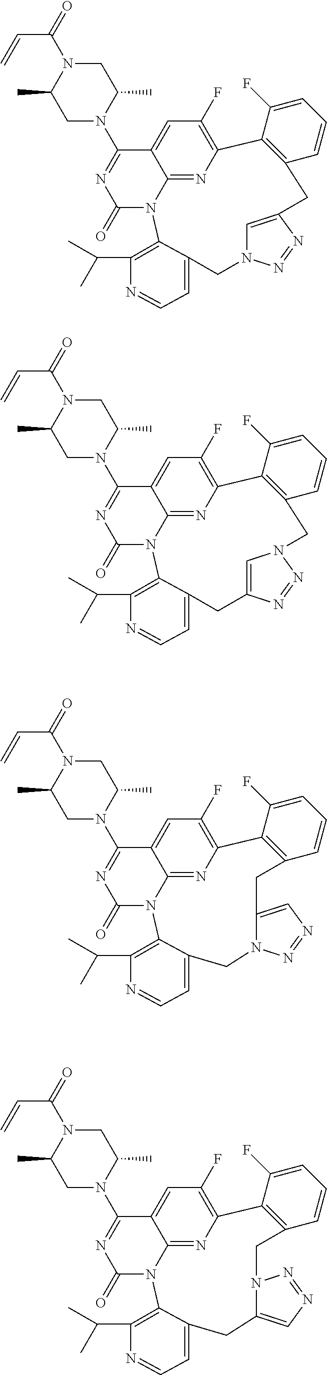 [SMILES: C=CC(=O)N1C[C@H](C)N(c2nc(=O)n3c4nc(c(F)cc24)-c2c(F)cccc2Cc2cn(nn2)Cc2ccnc(C(C)C)c2-3)C[C@H]1C ; C=CC(=O)N1C[C@H](C)N(c2nc(=O)n3c4nc(c(F)cc24)-c2c(F)cccc2Cn2cc(nn2)Cc2ccnc(C(C)C)c2-3)C[C@H]1C ; C=CC(=O)N1C[C@H](C)N(c2nc(=O)n3c4nc(c(F)cc24)-c2c(F)cccc2Cc2cnnn2Cc2ccnc(C(C)C)c2-3)C[C@H]1C ; C=CC(=O)N1C[C@H](C)N(c2nc(=O)n3c4nc(c(F)cc24)-c2c(F)cccc2Cn2nncc2Cc2ccnc(C(C)C)c2-3)C[C@H]1C]

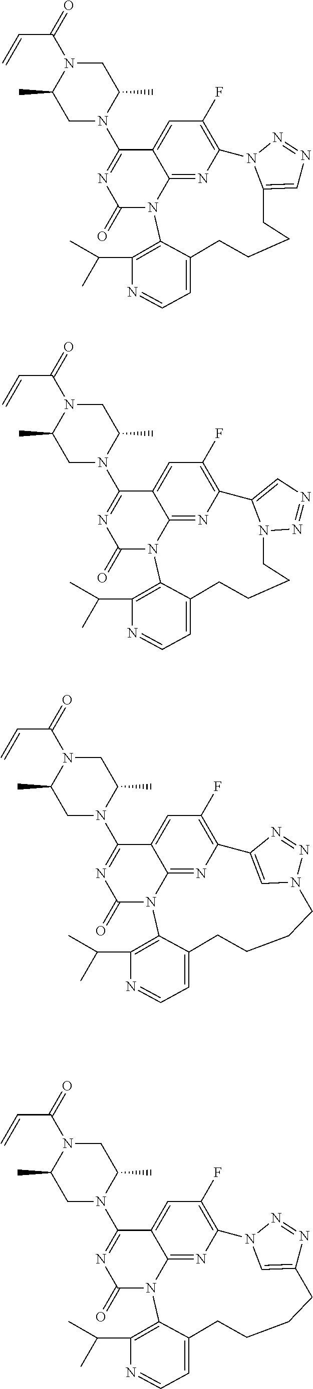 [SMILES: C=CC(=O)N1C[C@H](C)N(c2nc(=O)n3c4nc(c(F)cc24)-n2nncc2CCCCc2ccnc(C(C)C)c2-3)C[C@H]1C ; C=CC(=O)N1C[C@H](C)N(c2nc(=O)n3c4nc(c(F)cc24)-c2cnnn2CCCCc2ccnc(C(C)C)c2-3)C[C@H]1C ; C=CC(=O)N1C[C@H](C)N(c2nc(=O)n3c4nc(c(F)cc24)-c2cn(nn2)CCCCc2ccnc(C(C)C)c2-3)C[C@H]1C ; C=CC(=O)N1C[C@H](C)N(c2nc(=O)n3c4nc(c(F)cc24)-n2cc(nn2)CCCCc2ccnc(C(C)C)c2-3)C[C@H]1C]

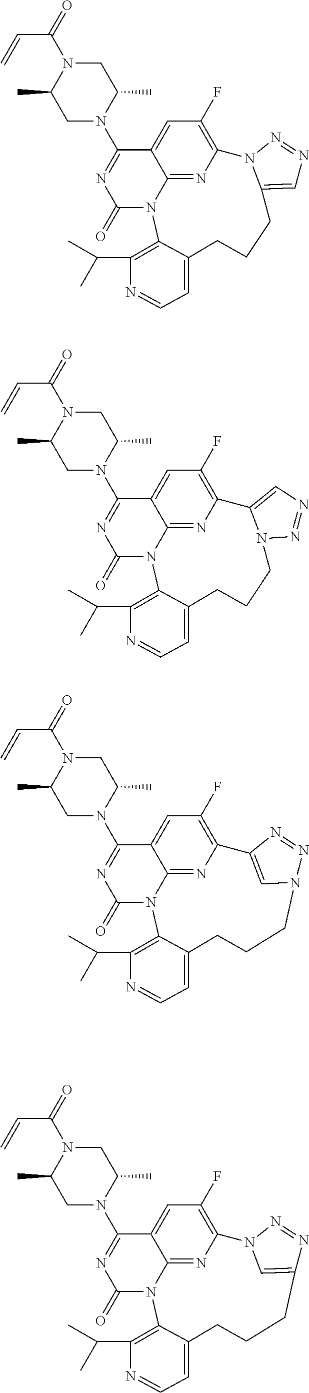 [SMILES: C=CC(=O)N1C[C@H](C)N(c2nc(=O)n3c4nc(c(F)cc24)-n2nncc2CCCc2ccnc(C(C)C)c2-3)C[C@H]1C ; C=CC(=O)N1C[C@H](C)N(c2nc(=O)n3c4nc(c(F)cc24)-c2cnnn2CCCc2ccnc(C(C)C)c2-3)C[C@H]1C ; C=CC(=O)N1C[C@H](C)N(c2nc(=O)n3c4nc(c(F)cc24)-c2cn(nn2)CCCc2ccnc(C(C)C)c2-3)C[C@H]1C ; C=CC(=O)N1C[C@H](C)N(c2nc(=O)n3c4nc(c(F)cc24)-n2cc(nn2)CCCc2ccnc(C(C)C)c2-3)C[C@H]1C]

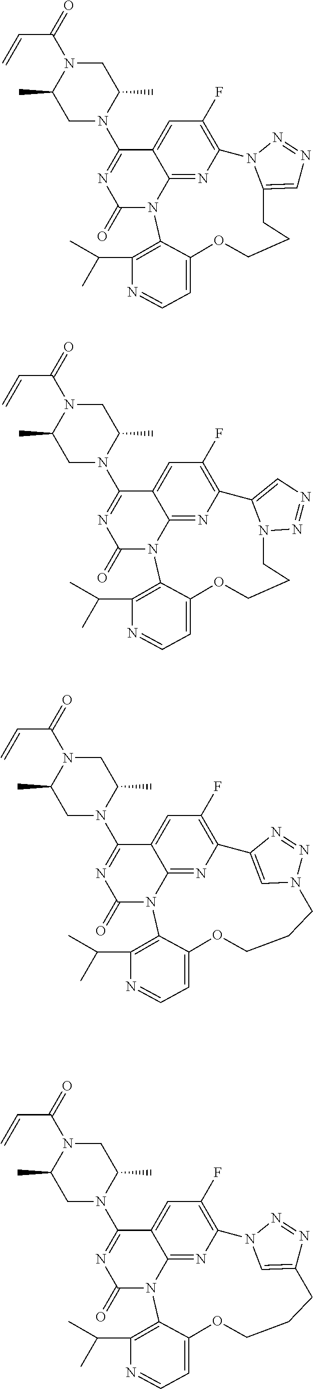 [SMILES: C=CC(=O)N1C[C@H](C)N(c2nc(=O)n3c4nc(c(F)cc24)-n2nncc2CCCOc2ccnc(C(C)C)c2-3)C[C@H]1C ; C=CC(=O)N1C[C@H](C)N(c2nc(=O)n3c4nc(c(F)cc24)-c2cnnn2CCCOc2ccnc(C(C)C)c2-3)C[C@H]1C ; C=CC(=O)N1C[C@H](C)N(c2nc(=O)n3c4nc(c(F)cc24)-c2cn(nn2)CCCOc2ccnc(C(C)C)c2-3)C[C@H]1C ; C=CC(=O)N1C[C@H](C)N(c2nc(=O)n3c4nc(c(F)cc24)-n2cc(nn2)CCCOc2ccnc(C(C)C)c2-3)C[C@H]1C]

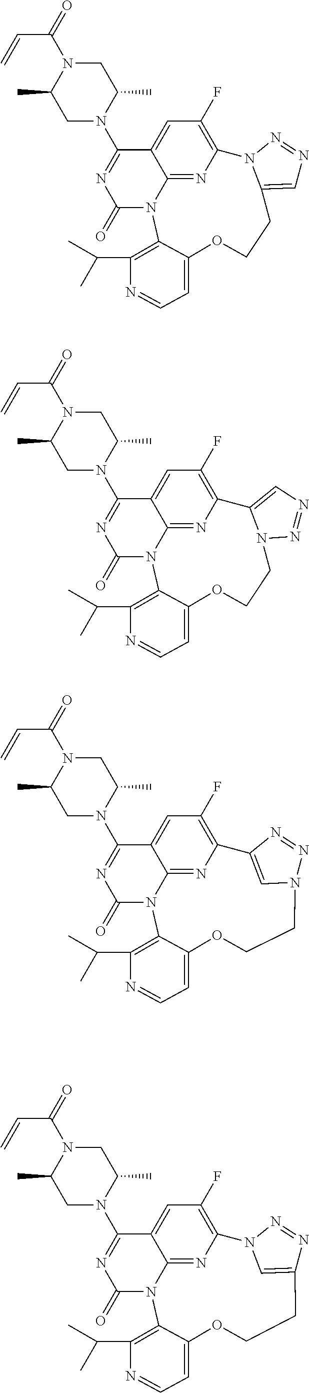 [SMILES: C=CC(=O)N1C[C@H](C)N(c2nc(=O)n3c4nc(c(F)cc24)-n2nncc2CCOc2ccnc(C(C)C)c2-3)C[C@H]1C ; C=CC(=O)N1C[C@H](C)N(c2nc(=O)n3c4nc(c(F)cc24)-c2cnnn2CCOc2ccnc(C(C)C)c2-3)C[C@H]1C ; C=CC(=O)N1C[C@H](C)N(c2nc(=O)n3c4nc(c(F)cc24)-c2cn(nn2)CCOc2ccnc(C(C)C)c2-3)C[C@H]1C ; C=CC(=O)N1C[C@H](C)N(c2nc(=O)n3c4nc(c(F)cc24)-n2cc(nn2)CCOc2ccnc(C(C)C)c2-3)C[C@H]1C]

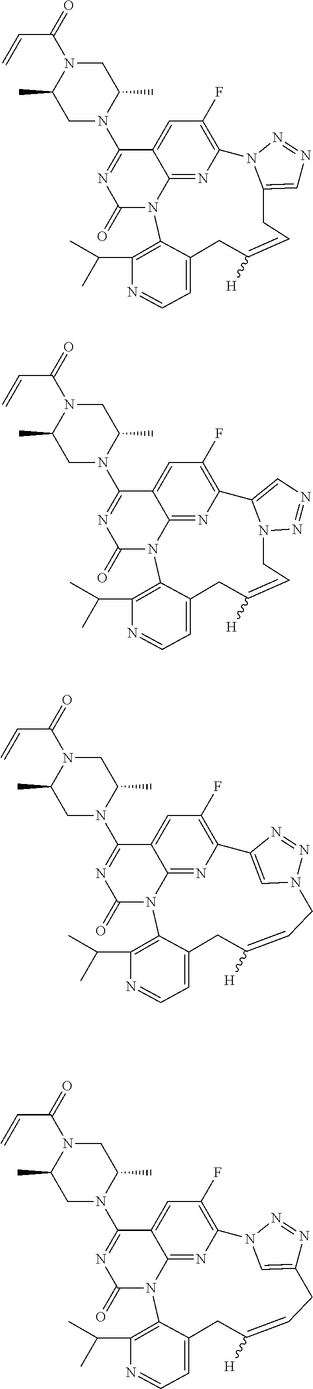 [SMILES: C=CC(=O)N1C[C@H](C)N(c2nc(=O)n3c4nc(c(F)cc24)-n2nncc2CC=CCc2ccnc(C(C)C)c2-3)C[C@H]1C ; C=CC(=O)N1C[C@H](C)N(c2nc(=O)n3c4nc(c(F)cc24)-c2cnnn2CC=CCc2ccnc(C(C)C)c2-3)C[C@H]1C ; C=CC(=O)N1C[C@H](C)N(c2nc(=O)n3c4nc(c(F)cc24)-c2cn(nn2)CC=CCc2ccnc(C(C)C)c2-3)C[C@H]1C ; C=CC(=O)N1C[C@H](C)N(c2nc(=O)n3c4nc(c(F)cc24)-n2cc(nn2)CC=CCc2ccnc(C(C)C)c2-3)C[C@H]1C]

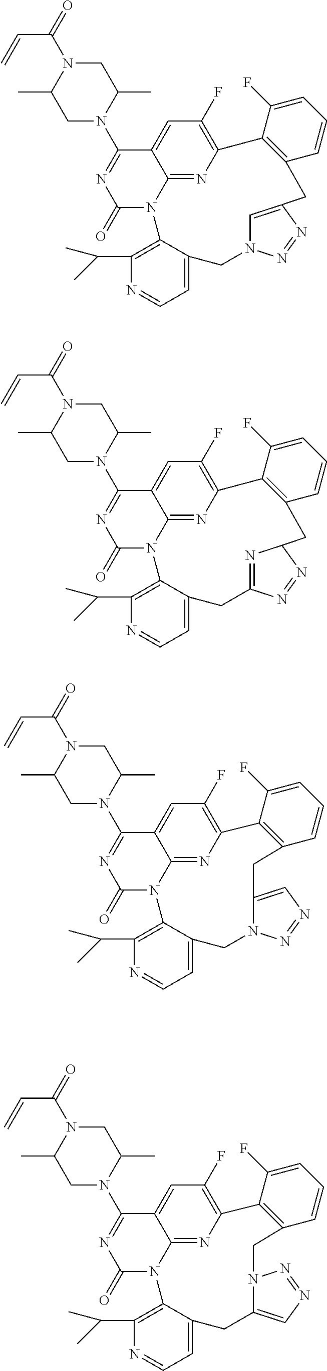 [SMILES: C=CC(=O)N1CC(C)N(c2nc(=O)n3c4nc(c(F)cc24)-c2c(F)cccc2Cc2cn(nn2)Cc2ccnc(C(C)C)c2-3)CC1C ; C=CC(=O)N1CC(C)N(c2nc(=O)n3c4nc(c(F)cc24)-c2c(F)cccc2CC2N=NC(=N2)Cc2ccnc(C(C)C)c2-3)CC1C ; C=CC(=O)N1CC(C)N(c2nc(=O)n3c4nc(c(F)cc24)-c2c(F)cccc2Cc2cnnn2Cc2ccnc(C(C)C)c2-3)CC1C ; C=CC(=O)N1CC(C)N(c2nc(=O)n3c4nc(c(F)cc24)-c2c(F)cccc2Cn2nncc2Cc2ccnc(C(C)C)c2-3)CC1C]

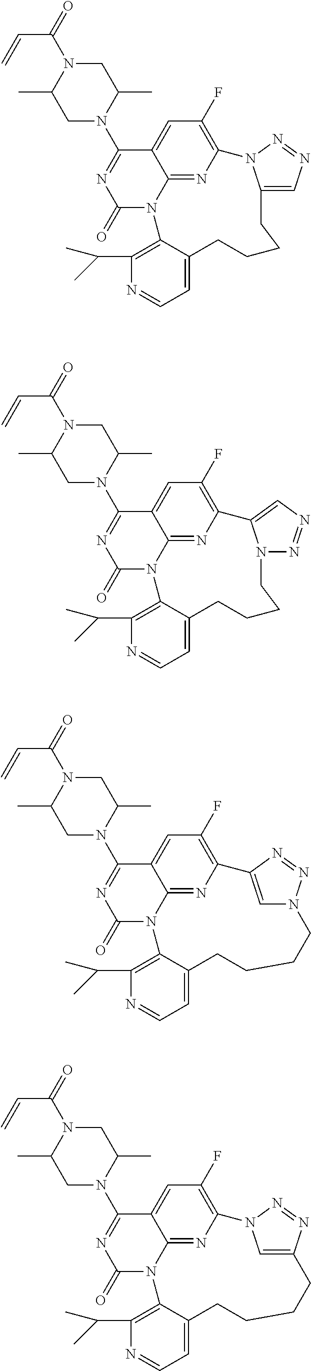 [SMILES: C=CC(=O)N1CC(C)N(c2nc(=O)n3c4nc(c(F)cc24)-n2nncc2CCCCc2ccnc(C(C)C)c2-3)CC1C ; C=CC(=O)N1CC(C)N(c2nc(=O)n3c4nc(c(F)cc24)-c2cnnn2CCCCc2ccnc(C(C)C)c2-3)CC1C ; C=CC(=O)N1CC(C)N(c2nc(=O)n3c4nc(c(F)cc24)-c2cn(nn2)CCCCc2ccnc(C(C)C)c2-3)CC1C ; C=CC(=O)N1CC(C)N(c2nc(=O)n3c4nc(c(F)cc24)-n2cc(nn2)CCCCc2ccnc(C(C)C)c2-3)CC1C]

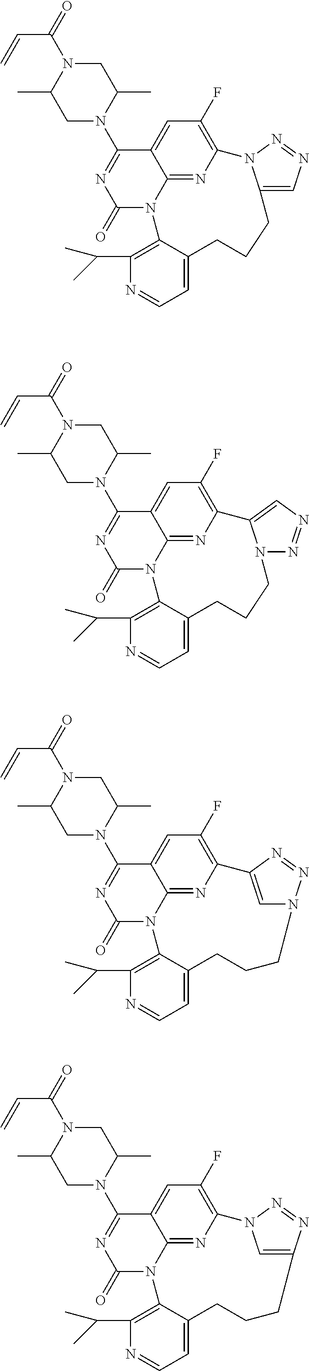 [SMILES: C=CC(=O)N1CC(C)N(c2nc(=O)n3c4nc(c(F)cc24)-n2nncc2CCCc2ccnc(C(C)C)c2-3)CC1C ; C=CC(=O)N1CC(C)N(c2nc(=O)n3c4nc(c(F)cc24)-c2cnnn2CCCc2ccnc(C(C)C)c2-3)CC1C ; C=CC(=O)N1CC(C)N(c2nc(=O)n3c4nc(c(F)cc24)-c2cn(nn2)CCCc2ccnc(C(C)C)c2-3)CC1C ; C=CC(=O)N1CC(C)N(c2nc(=O)n3c4nc(c(F)cc24)-n2cc(nn2)CCCc2ccnc(C(C)C)c2-3)CC1C]

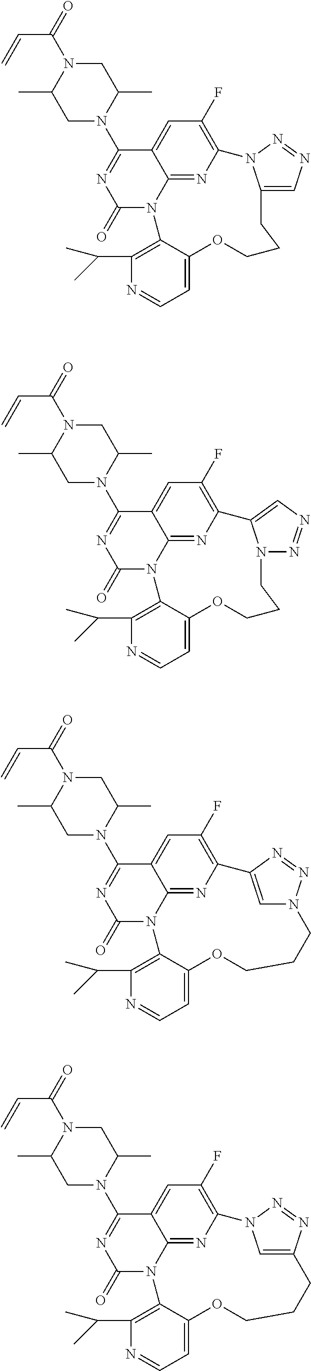 [SMILES: C=CC(=O)N1CC(C)N(c2nc(=O)n3c4nc(c(F)cc24)-n2nncc2CCCOc2ccnc(C(C)C)c2-3)CC1C ; C=CC(=O)N1CC(C)N(c2nc(=O)n3c4nc(c(F)cc24)-c2cnnn2CCCOc2ccnc(C(C)C)c2-3)CC1C ; C=CC(=O)N1CC(C)N(c2nc(=O)n3c4nc(c(F)cc24)-c2cn(nn2)CCCOc2ccnc(C(C)C)c2-3)CC1C ; C=CC(=O)N1CC(C)N(c2nc(=O)n3c4nc(c(F)cc24)-n2cc(nn2)CCCOc2ccnc(C(C)C)c2-3)CC1C]

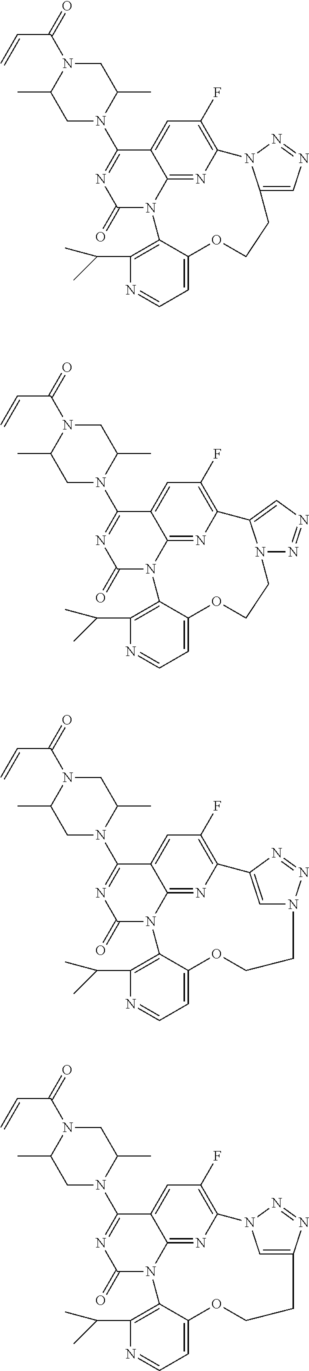 [SMILES: C=CC(=O)N1CC(C)N(c2nc(=O)n3c4nc(c(F)cc24)-n2nncc2CCOc2ccnc(C(C)C)c2-3)CC1C ; C=CC(=O)N1CC(C)N(c2nc(=O)n3c4nc(c(F)cc24)-c2cnnn2CCOc2ccnc(C(C)C)c2-3)CC1C ; C=CC(=O)N1CC(C)N(c2nc(=O)n3c4nc(c(F)cc24)-c2cn(nn2)CCOc2ccnc(C(C)C)c2-3)CC1C ; C=CC(=O)N1CC(C)N(c2nc(=O)n3c4nc(c(F)cc24)-n2cc(nn2)CCOc2ccnc(C(C)C)c2-3)CC1C]

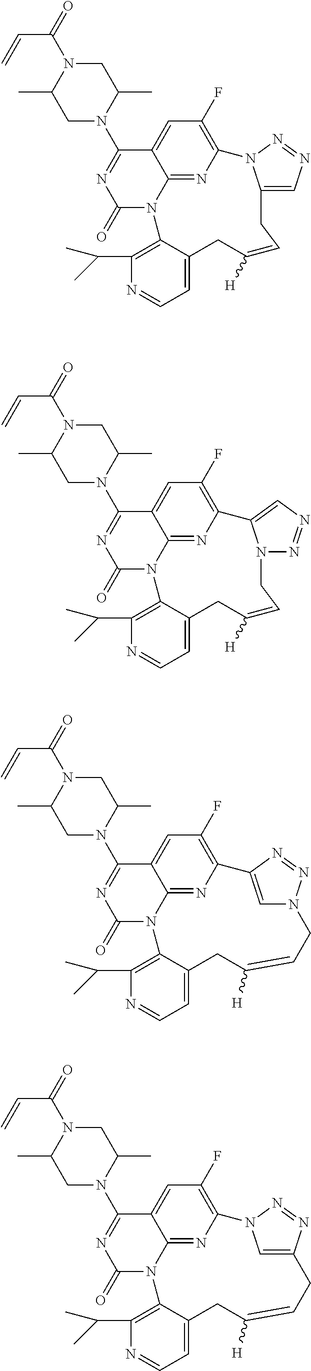 [SMILES: C=CC(=O)N1CC(C)N(c2nc(=O)n3c4nc(c(F)cc24)-n2nncc2CC=CCc2ccnc(C(C)C)c2-3)CC1C ; C=CC(=O)N1CC(C)N(c2nc(=O)n3c4nc(c(F)cc24)-c2cnnn2CC=CCc2ccnc(C(C)C)c2-3)CC1C ; C=CC(=O)N1CC(C)N(c2nc(=O)n3c4nc(c(F)cc24)-c2cn(nn2)CC=CCc2ccnc(C(C)C)c2-3)CC1C ; C=CC(=O)N1CC(C)N(c2nc(=O)n3c4nc(c(F)cc24)-n2cc(nn2)CC=CCc2ccnc(C(C)C)c2-3)CC1C]

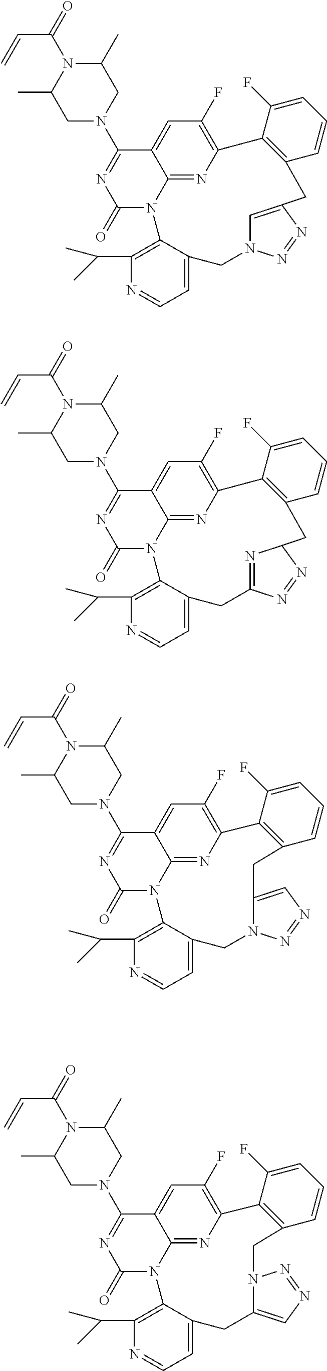 [SMILES: C=CC(=O)N1C(C)CN(c2nc(=O)n3c4nc(c(F)cc24)-c2c(F)cccc2Cc2cn(nn2)Cc2ccnc(C(C)C)c2-3)CC1C ; C=CC(=O)N1C(C)CN(c2nc(=O)n3c4nc(c(F)cc24)-c2c(F)cccc2CC2N=NC(=N2)Cc2ccnc(C(C)C)c2-3)CC1C ; C=CC(=O)N1C(C)CN(c2nc(=O)n3c4nc(c(F)cc24)-c2c(F)cccc2Cc2cnnn2Cc2ccnc(C(C)C)c2-3)CC1C ; C=CC(=O)N1C(C)CN(c2nc(=O)n3c4nc(c(F)cc24)-c2c(F)cccc2Cn2nncc2Cc2ccnc(C(C)C)c2-3)CC1C]

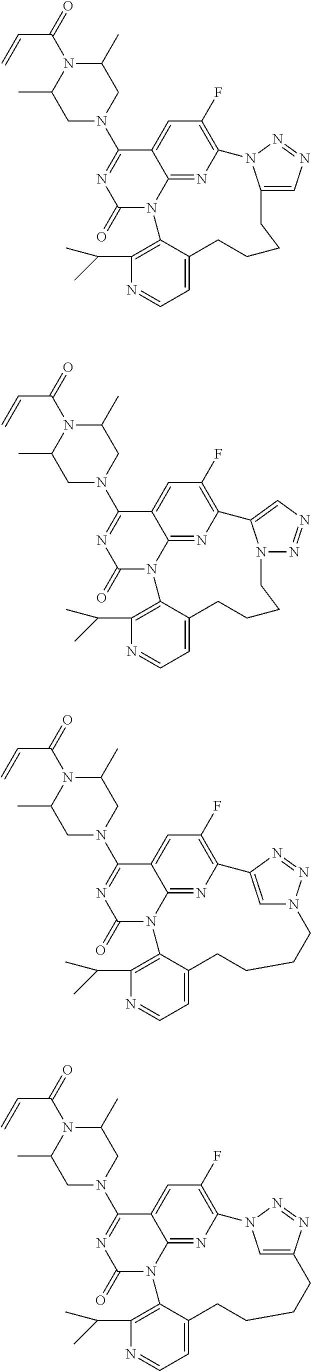 [SMILES: C=CC(=O)N1C(C)CN(c2nc(=O)n3c4nc(c(F)cc24)-n2nncc2CCCCc2ccnc(C(C)C)c2-3)CC1C ; C=CC(=O)N1C(C)CN(c2nc(=O)n3c4nc(c(F)cc24)-c2cnnn2CCCCc2ccnc(C(C)C)c2-3)CC1C ; C=CC(=O)N1C(C)CN(c2nc(=O)n3c4nc(c(F)cc24)-c2cn(nn2)CCCCc2ccnc(C(C)C)c2-3)CC1C ; C=CC(=O)N1C(C)CN(c2nc(=O)n3c4nc(c(F)cc24)-n2cc(nn2)CCCCc2ccnc(C(C)C)c2-3)CC1C]

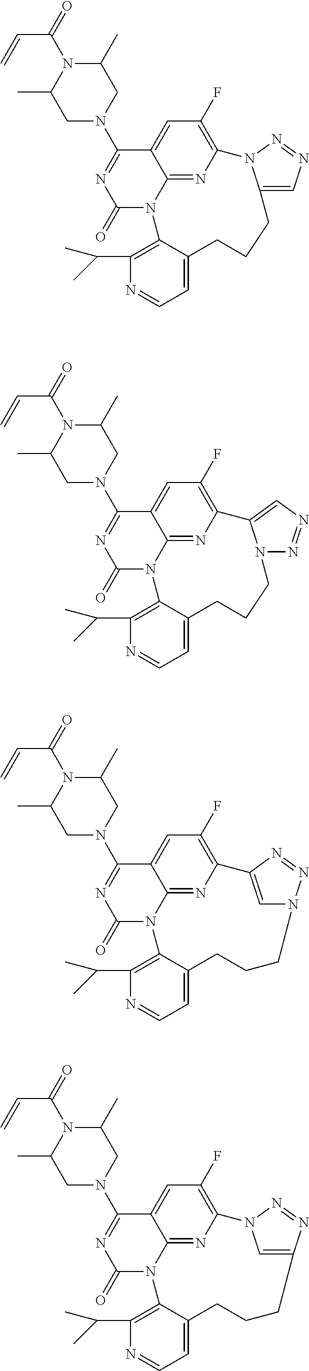 [SMILES: C=CC(=O)N1C(C)CN(c2nc(=O)n3c4nc(c(F)cc24)-n2nncc2CCCc2ccnc(C(C)C)c2-3)CC1C ; C=CC(=O)N1C(C)CN(c2nc(=O)n3c4nc(c(F)cc24)-c2cnnn2CCCc2ccnc(C(C)C)c2-3)CC1C ; C=CC(=O)N1C(C)CN(c2nc(=O)n3c4nc(c(F)cc24)-c2cn(nn2)CCCc2ccnc(C(C)C)c2-3)CC1C ; C=CC(=O)N1C(C)CN(c2nc(=O)n3c4nc(c(F)cc24)-n2cc(nn2)CCCc2ccnc(C(C)C)c2-3)CC1C]

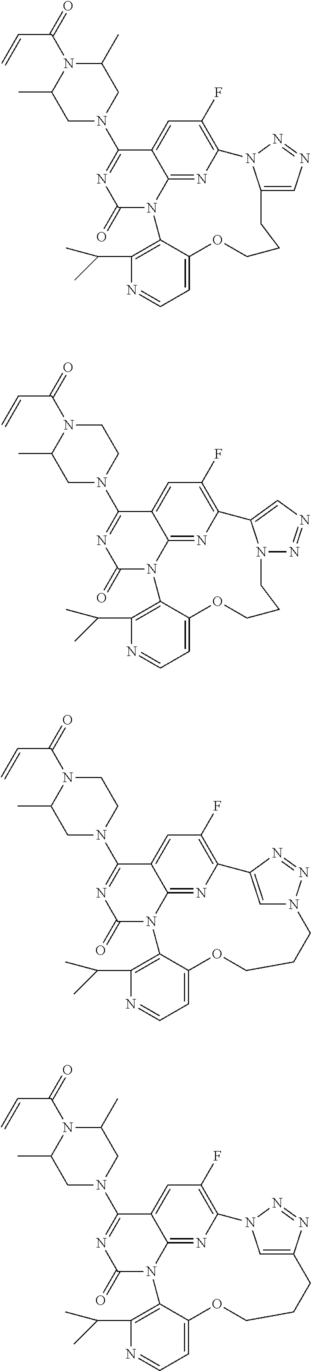 [SMILES: C=CC(=O)N1C(C)CN(c2nc(=O)n3c4nc(c(F)cc24)-n2nncc2CCCOc2ccnc(C(C)C)c2-3)CC1C ; C=CC(=O)N1CCN(c2nc(=O)n3c4nc(c(F)cc24)-c2cnnn2CCCOc2ccnc(C(C)C)c2-3)CC1C ; C=CC(=O)N1CCN(c2nc(=O)n3c4nc(c(F)cc24)-c2cn(nn2)CCCOc2ccnc(C(C)C)c2-3)CC1C ; C=CC(=O)N1C(C)CN(c2nc(=O)n3c4nc(c(F)cc24)-n2cc(nn2)CCCOc2ccnc(C(C)C)c2-3)CC1C]

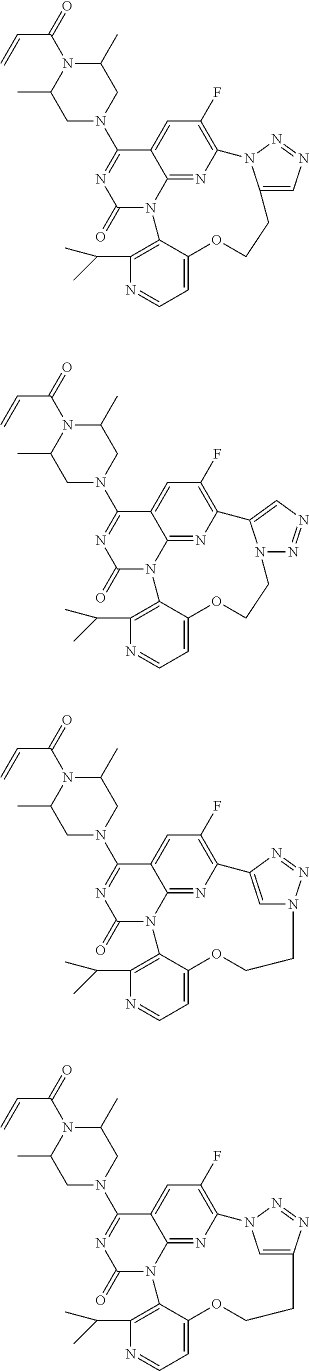 [SMILES: C=CC(=O)N1C(C)CN(c2nc(=O)n3c4nc(c(F)cc24)-n2nncc2CCOc2ccnc(C(C)C)c2-3)CC1C ; C=CC(=O)N1C(C)CN(c2nc(=O)n3c4nc(c(F)cc24)-c2cnnn2CCOc2ccnc(C(C)C)c2-3)CC1C ; C=CC(=O)N1C(C)CN(c2nc(=O)n3c4nc(c(F)cc24)-c2cn(nn2)CCOc2ccnc(C(C)C)c2-3)CC1C ; C=CC(=O)N1C(C)CN(c2nc(=O)n3c4nc(c(F)cc24)-n2cc(nn2)CCOc2ccnc(C(C)C)c2-3)CC1C]

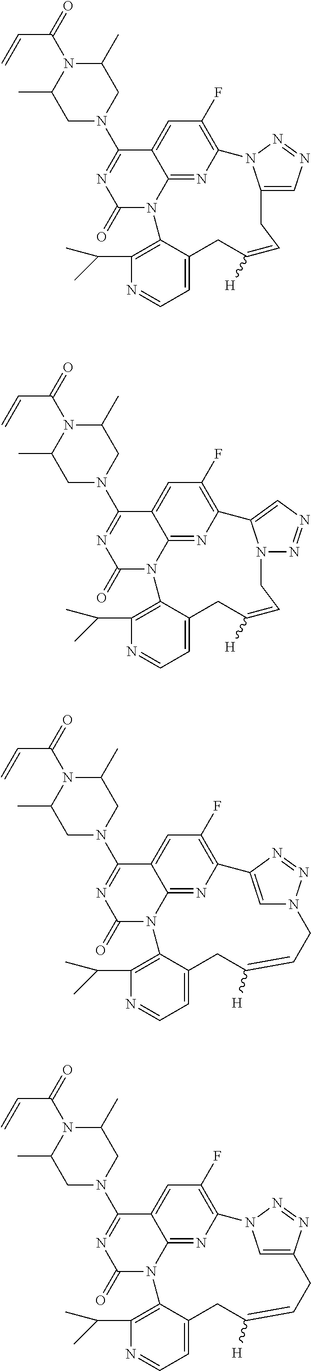 [SMILES: C=CC(=O)N1C(C)CN(c2nc(=O)n3c4nc(c(F)cc24)-n2nncc2CC=CCc2ccnc(C(C)C)c2-3)CC1C ; C=CC(=O)N1C(C)CN(c2nc(=O)n3c4nc(c(F)cc24)-c2cnnn2CC=CCc2ccnc(C(C)C)c2-3)CC1C ; C=CC(=O)N1C(C)CN(c2nc(=O)n3c4nc(c(F)cc24)-c2cn(nn2)CC=CCc2ccnc(C(C)C)c2-3)CC1C ; C=CC(=O)N1C(C)CN(c2nc(=O)n3c4nc(c(F)cc24)-n2cc(nn2)CC=CCc2ccnc(C(C)C)c2-3)CC1C]

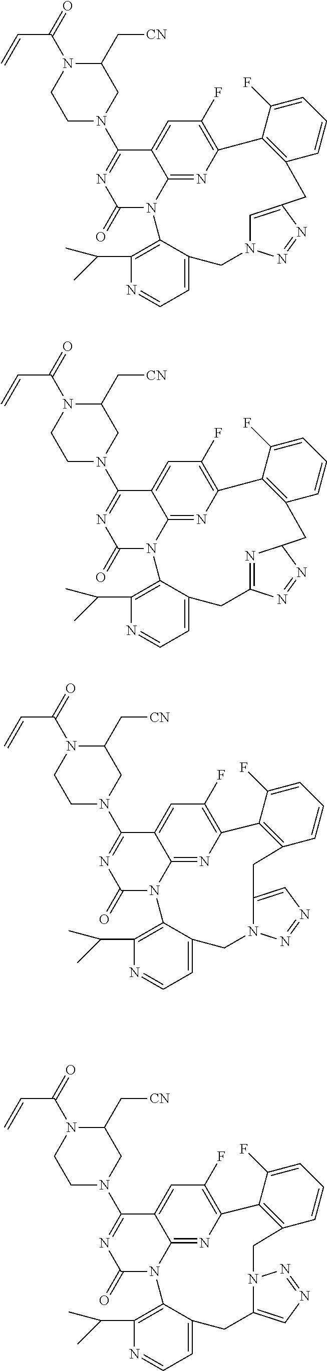 [SMILES: C=CC(=O)N1CCN(c2nc(=O)n3c4nc(c(F)cc24)-c2c(F)cccc2Cc2cn(nn2)Cc2ccnc(C(C)C)c2-3)CC1CC#N ; C=CC(=O)N1CCN(c2nc(=O)n3c4nc(c(F)cc24)-c2c(F)cccc2CC2N=NC(=N2)Cc2ccnc(C(C)C)c2-3)CC1CC#N ; C=CC(=O)N1CCN(c2nc(=O)n3c4nc(c(F)cc24)-c2c(F)cccc2Cc2cnnn2Cc2ccnc(C(C)C)c2-3)CC1CC#N ; C=CC(=O)N1CCN(c2nc(=O)n3c4nc(c(F)cc24)-c2c(F)cccc2Cn2nncc2Cc2ccnc(C(C)C)c2-3)CC1CC#N]

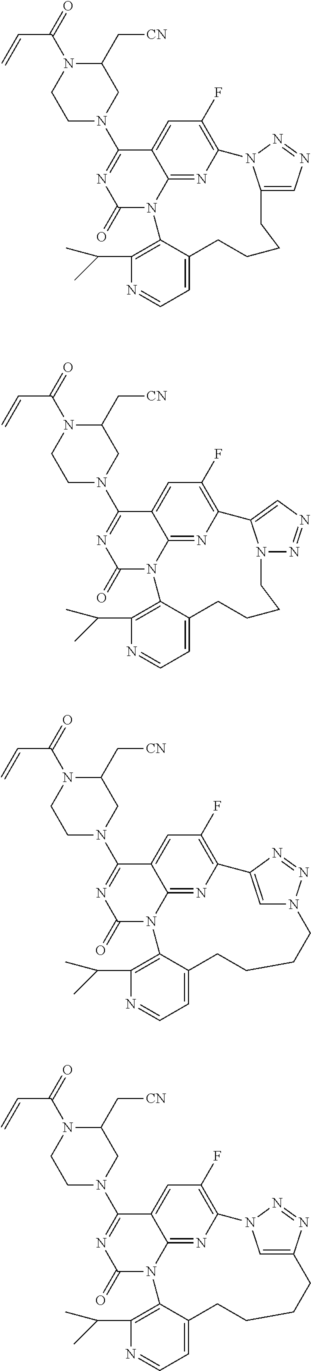 [SMILES: C=CC(=O)N1CCN(c2nc(=O)n3c4nc(c(F)cc24)-n2nncc2CCCCc2ccnc(C(C)C)c2-3)CC1CC#N ; C=CC(=O)N1CCN(c2nc(=O)n3c4nc(c(F)cc24)-c2cnnn2CCCCc2ccnc(C(C)C)c2-3)CC1CC#N ; C=CC(=O)N1CCN(c2nc(=O)n3c4nc(c(F)cc24)-c2cn(nn2)CCCCc2ccnc(C(C)C)c2-3)CC1CC#N ; C=CC(=O)N1CCN(c2nc(=O)n3c4nc(c(F)cc24)-n2cc(nn2)CCCCc2ccnc(C(C)C)c2-3)CC1CC#N]

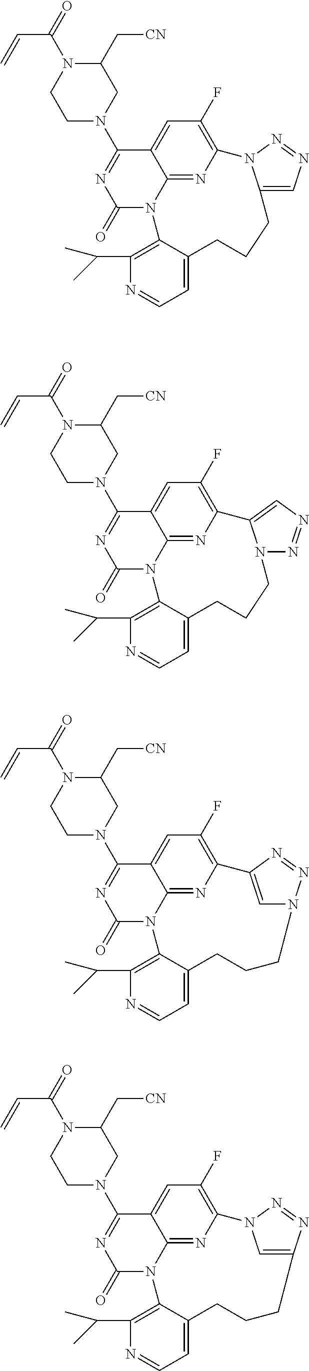 [SMILES: C=CC(=O)N1CCN(c2nc(=O)n3c4nc(c(F)cc24)-n2nncc2CCCc2ccnc(C(C)C)c2-3)CC1CC#N ; C=CC(=O)N1CCN(c2nc(=O)n3c4nc(c(F)cc24)-c2cnnn2CCCc2ccnc(C(C)C)c2-3)CC1CC#N ; C=CC(=O)N1CCN(c2nc(=O)n3c4nc(c(F)cc24)-c2cn(nn2)CCCc2ccnc(C(C)C)c2-3)CC1CC#N ; C=CC(=O)N1CCN(c2nc(=O)n3c4nc(c(F)cc24)-n2cc(nn2)CCCc2ccnc(C(C)C)c2-3)CC1CC#N]

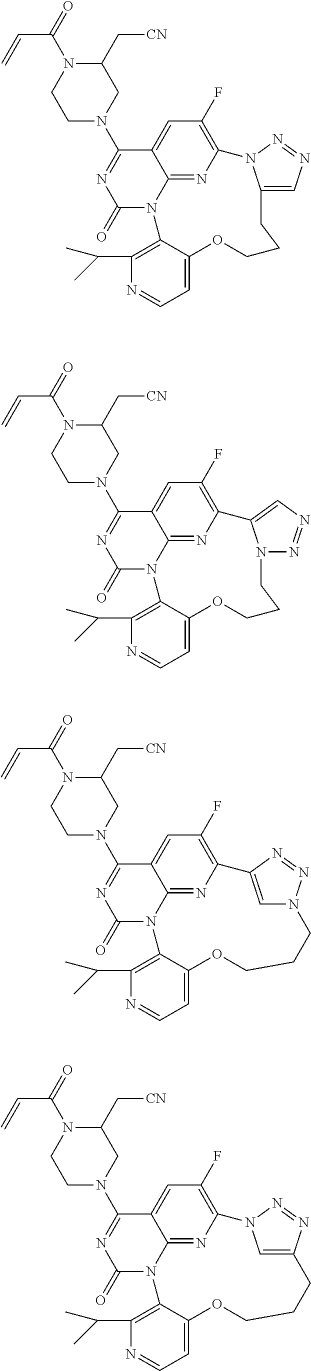 [SMILES: C=CC(=O)N1CCN(c2nc(=O)n3c4nc(c(F)cc24)-n2nncc2CCCOc2ccnc(C(C)C)c2-3)CC1CC#N ; C=CC(=O)N1CCN(c2nc(=O)n3c4nc(c(F)cc24)-c2cnnn2CCCOc2ccnc(C(C)C)c2-3)CC1CC#N ; C=CC(=O)N1CCN(c2nc(=O)n3c4nc(c(F)cc24)-c2cn(nn2)CCCOc2ccnc(C(C)C)c2-3)CC1CC#N ; C=CC(=O)N1CCN(c2nc(=O)n3c4nc(c(F)cc24)-n2cc(nn2)CCCOc2ccnc(C(C)C)c2-3)CC1CC#N]

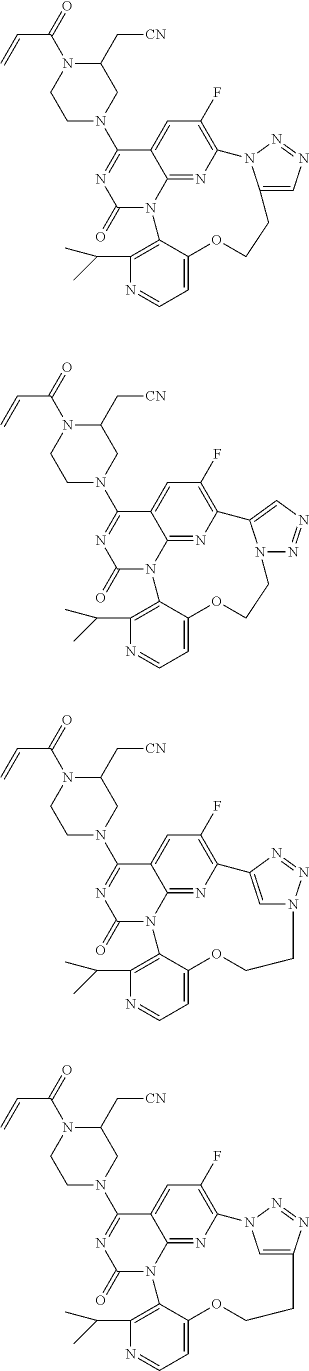 [SMILES: C=CC(=O)N1CCN(c2nc(=O)n3c4nc(c(F)cc24)-n2nncc2CCOc2ccnc(C(C)C)c2-3)CC1CC#N ; C=CC(=O)N1CCN(c2nc(=O)n3c4nc(c(F)cc24)-c2cnnn2CCOc2ccnc(C(C)C)c2-3)CC1CC#N ; C=CC(=O)N1CCN(c2nc(=O)n3c4nc(c(F)cc24)-c2cn(nn2)CCOc2ccnc(C(C)C)c2-3)CC1CC#N ; C=CC(=O)N1CCN(c2nc(=O)n3c4nc(c(F)cc24)-n2cc(nn2)CCOc2ccnc(C(C)C)c2-3)CC1CC#N]

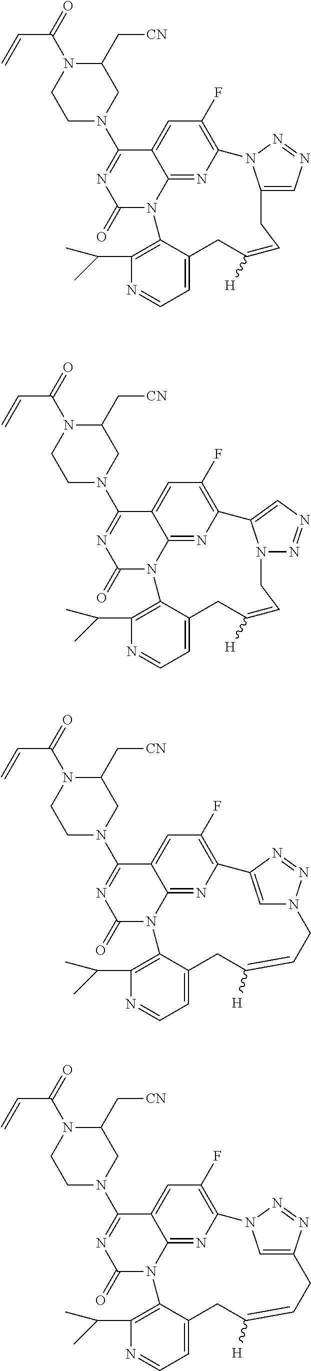 [SMILES: C=CC(=O)N1CCN(c2nc(=O)n3c4nc(c(F)cc24)-n2nncc2CC=CCc2ccnc(C(C)C)c2-3)CC1CC#N ; C=CC(=O)N1CCN(c2nc(=O)n3c4nc(c(F)cc24)-c2cnnn2CC=CCc2ccnc(C(C)C)c2-3)CC1CC#N ; C=CC(=O)N1CCN(c2nc(=O)n3c4nc(c(F)cc24)-c2cn(nn2)CC=CCc2ccnc(C(C)C)c2-3)CC1CC#N ; C=CC(=O)N1CCN(c2nc(=O)n3c4nc(c(F)cc24)-n2cc(nn2)CC=CCc2ccnc(C(C)C)c2-3)CC1CC#N]

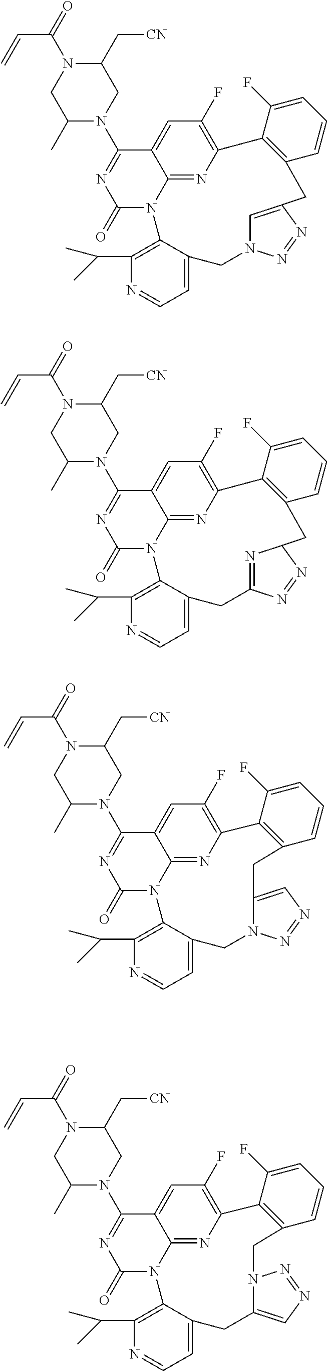 [SMILES: C=CC(=O)N1CC(C)N(c2nc(=O)n3c4nc(c(F)cc24)-c2c(F)cccc2Cc2cn(nn2)Cc2ccnc(C(C)C)c2-3)CC1CC#N ; C=CC(=O)N1CC(C)N(c2nc(=O)n3c4nc(c(F)cc24)-c2c(F)cccc2CC2N=NC(=N2)Cc2ccnc(C(C)C)c2-3)CC1CC#N ; C=CC(=O)N1CC(C)N(c2nc(=O)n3c4nc(c(F)cc24)-c2c(F)cccc2Cc2cnnn2Cc2ccnc(C(C)C)c2-3)CC1CC#N ; C=CC(=O)N1CC(C)N(c2nc(=O)n3c4nc(c(F)cc24)-c2c(F)cccc2Cn2nncc2Cc2ccnc(C(C)C)c2-3)CC1CC#N]

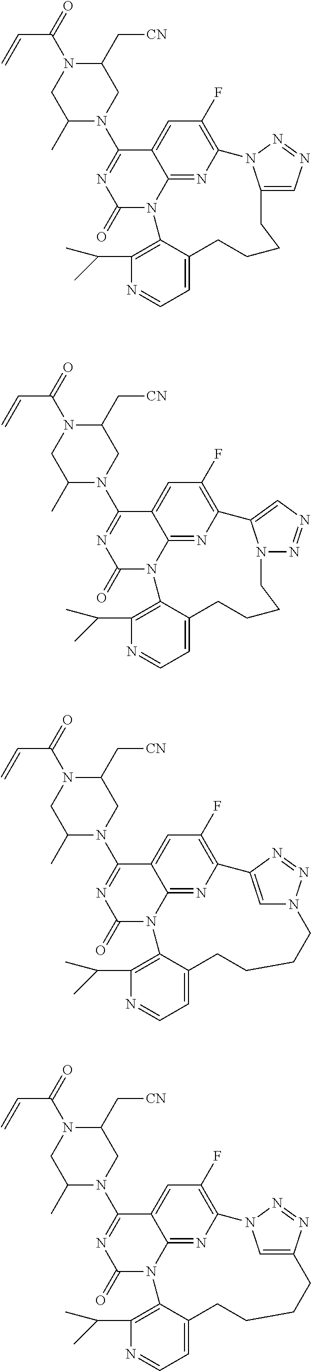 [SMILES: C=CC(=O)N1CC(C)N(c2nc(=O)n3c4nc(c(F)cc24)-n2nncc2CCCCc2ccnc(C(C)C)c2-3)CC1CC#N ; C=CC(=O)N1CC(C)N(c2nc(=O)n3c4nc(c(F)cc24)-c2cnnn2CCCCc2ccnc(C(C)C)c2-3)CC1CC#N ; C=CC(=O)N1CC(C)N(c2nc(=O)n3c4nc(c(F)cc24)-c2cn(nn2)CCCCc2ccnc(C(C)C)c2-3)CC1CC#N ; C=CC(=O)N1CC(C)N(c2nc(=O)n3c4nc(c(F)cc24)-n2cc(nn2)CCCCc2ccnc(C(C)C)c2-3)CC1CC#N]

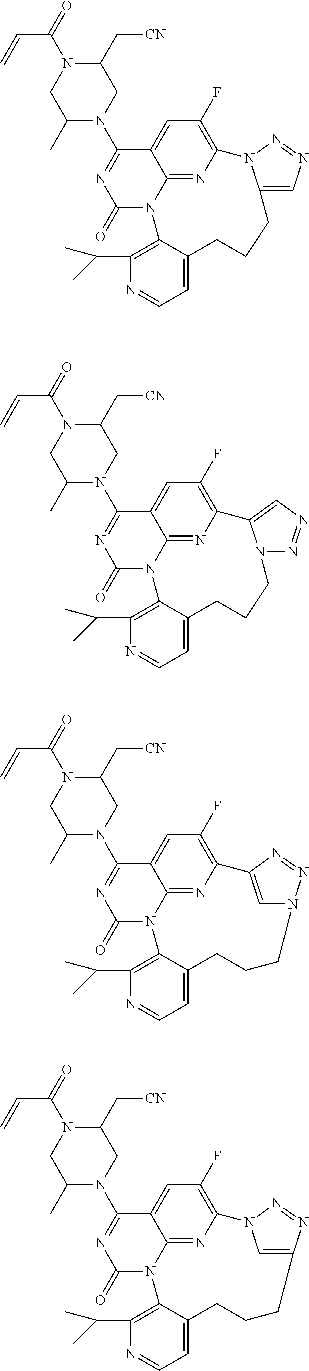 [SMILES: C=CC(=O)N1CC(C)N(c2nc(=O)n3c4nc(c(F)cc24)-n2nncc2CCCc2ccnc(C(C)C)c2-3)CC1CC#N ; C=CC(=O)N1CC(C)N(c2nc(=O)n3c4nc(c(F)cc24)-c2cnnn2CCCc2ccnc(C(C)C)c2-3)CC1CC#N ; C=CC(=O)N1CC(C)N(c2nc(=O)n3c4nc(c(F)cc24)-c2cn(nn2)CCCc2ccnc(C(C)C)c2-3)CC1CC#N ; C=CC(=O)N1CC(C)N(c2nc(=O)n3c4nc(c(F)cc24)-n2cc(nn2)CCCc2ccnc(C(C)C)c2-3)CC1CC#N]

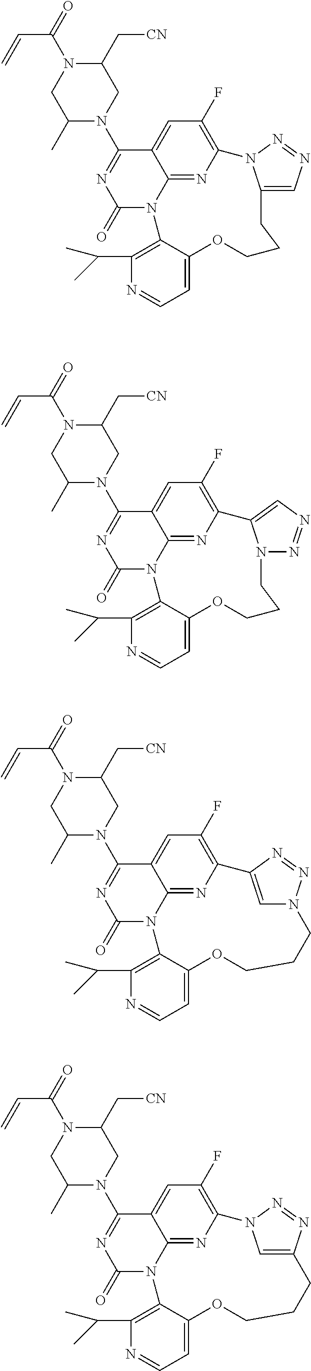 [SMILES: C=CC(=O)N1CC(C)N(c2nc(=O)n3c4nc(c(F)cc24)-n2nncc2CCCOc2ccnc(C(C)C)c2-3)CC1CC#N ; C=CC(=O)N1CC(C)N(c2nc(=O)n3c4nc(c(F)cc24)-c2cnnn2CCCOc2ccnc(C(C)C)c2-3)CC1CC#N ; C=CC(=O)N1CC(C)N(c2nc(=O)n3c4nc(c(F)cc24)-c2cn(nn2)CCCOc2ccnc(C(C)C)c2-3)CC1CC#N ; C=CC(=O)N1CC(C)N(c2nc(=O)n3c4nc(c(F)cc24)-n2cc(nn2)CCCOc2ccnc(C(C)C)c2-3)CC1CC#N]

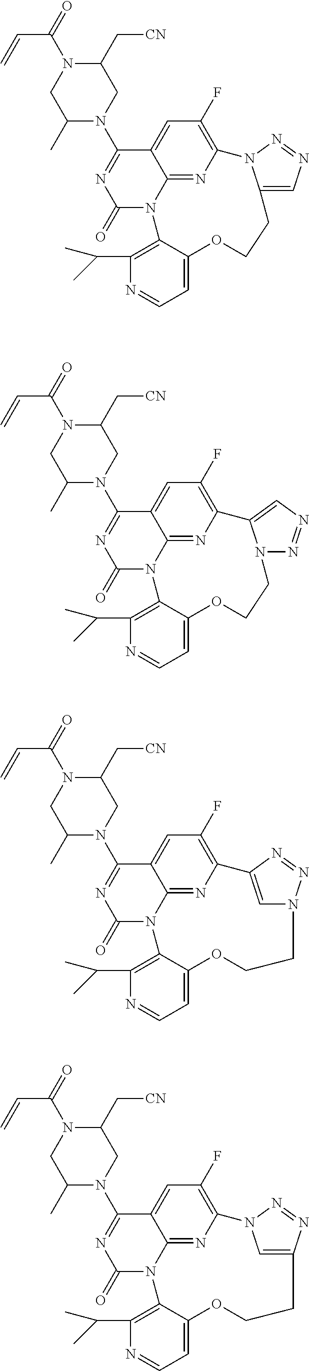 [SMILES: C=CC(=O)N1CC(C)N(c2nc(=O)n3c4nc(c(F)cc24)-n2nncc2CCOc2ccnc(C(C)C)c2-3)CC1CC#N ; C=CC(=O)N1CC(C)N(c2nc(=O)n3c4nc(c(F)cc24)-c2cnnn2CCOc2ccnc(C(C)C)c2-3)CC1CC#N ; C=CC(=O)N1CC(C)N(c2nc(=O)n3c4nc(c(F)cc24)-c2cn(nn2)CCOc2ccnc(C(C)C)c2-3)CC1CC#N ; C=CC(=O)N1CC(C)N(c2nc(=O)n3c4nc(c(F)cc24)-n2cc(nn2)CCOc2ccnc(C(C)C)c2-3)CC1CC#N]

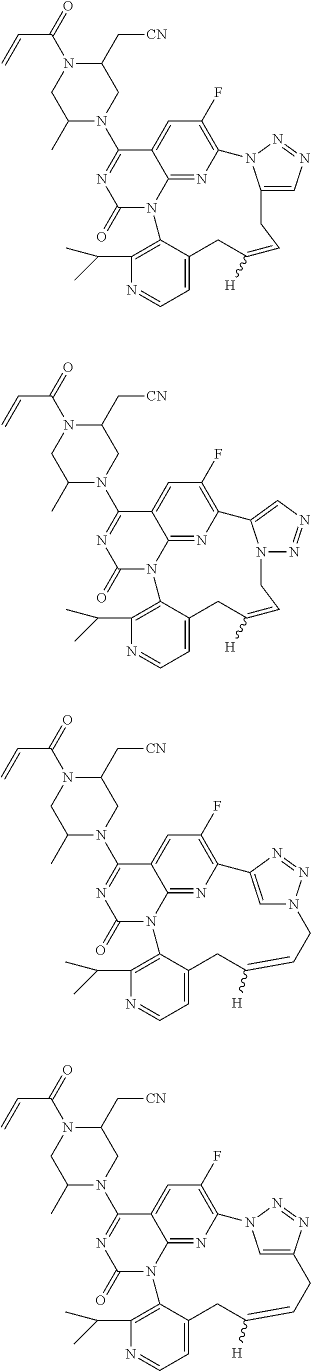 [SMILES: C=CC(=O)N1CC(C)N(c2nc(=O)n3c4nc(c(F)cc24)-n2nncc2CC=CCc2ccnc(C(C)C)c2-3)CC1CC#N ; C=CC(=O)N1CC(C)N(c2nc(=O)n3c4nc(c(F)cc24)-c2cnnn2CC=CCc2ccnc(C(C)C)c2-3)CC1CC#N ; C=CC(=O)N1CC(C)N(c2nc(=O)n3c4nc(c(F)cc24)-c2cn(nn2)CC=CCc2ccnc(C(C)C)c2-3)CC1CC#N ; C=CC(=O)N1CC(C)N(c2nc(=O)n3c4nc(c(F)cc24)-n2cc(nn2)CC=CCc2ccnc(C(C)C)c2-3)CC1CC#N]

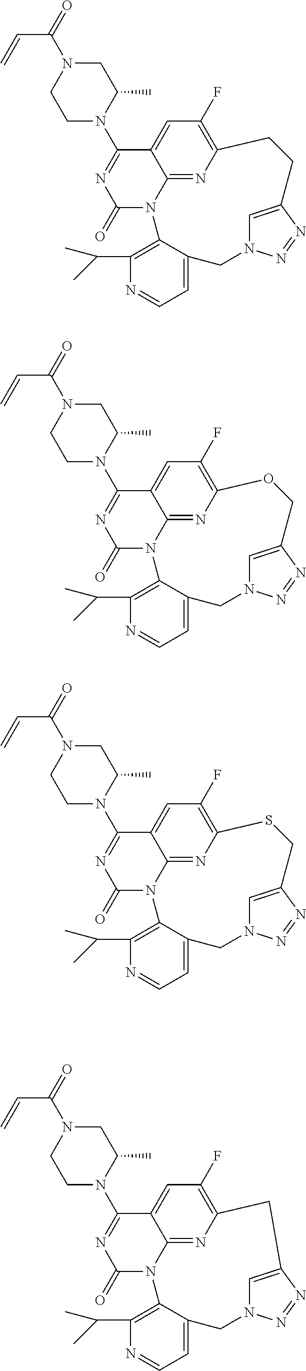 [SMILES: C=CC(=O)N1CCN(c2nc(=O)n3c4nc(c(F)cc24)CCc2cn(nn2)Cc2ccnc(C(C)C)c2-3)[C@@H](C)C1 ; C=CC(=O)N1CCN(c2nc(=O)n3c4nc(c(F)cc24)OCc2cn(nn2)Cc2ccnc(C(C)C)c2-3)[C@@H](C)C1 ; C=CC(=O)N1CCN(c2nc(=O)n3c4nc(c(F)cc24)SCc2cn(nn2)Cc2ccnc(C(C)C)c2-3)[C@@H](C)C1 ; C=CC(=O)N1CCN(c2nc(=O)n3c4nc(c(F)cc24)Cc2cn(nn2)Cc2ccnc(C(C)C)c2-3)[C@@H](C)C1]

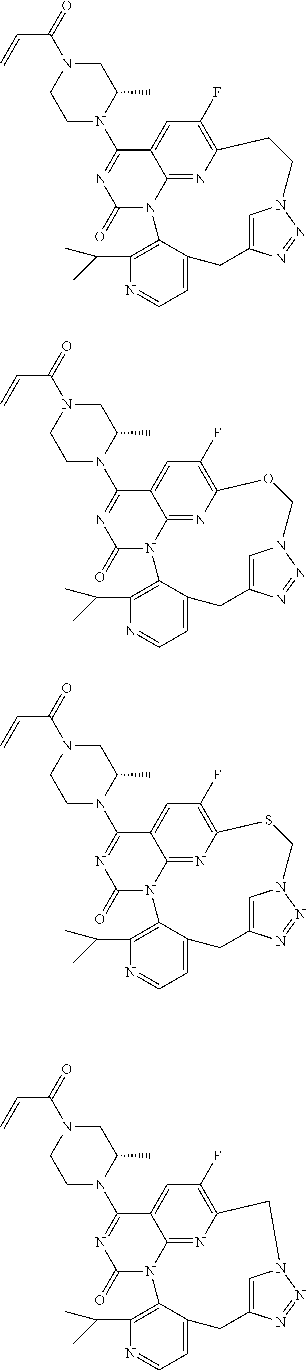 [SMILES: C=CC(=O)N1CCN(c2nc(=O)n3c4nc(c(F)cc24)CCn2cc(nn2)Cc2ccnc(C(C)C)c2-3)[C@@H](C)C1 ; C=CC(=O)N1CCN(c2nc(=O)n3c4nc(c(F)cc24)OCn2cc(nn2)Cc2ccnc(C(C)C)c2-3)[C@@H](C)C1 ; C=CC(=O)N1CCN(c2nc(=O)n3c4nc(c(F)cc24)SCn2cc(nn2)Cc2ccnc(C(C)C)c2-3)[C@@H](C)C1 ; C=CC(=O)N1CCN(c2nc(=O)n3c4nc(c(F)cc24)Cn2cc(nn2)Cc2ccnc(C(C)C)c2-3)[C@@H](C)C1]

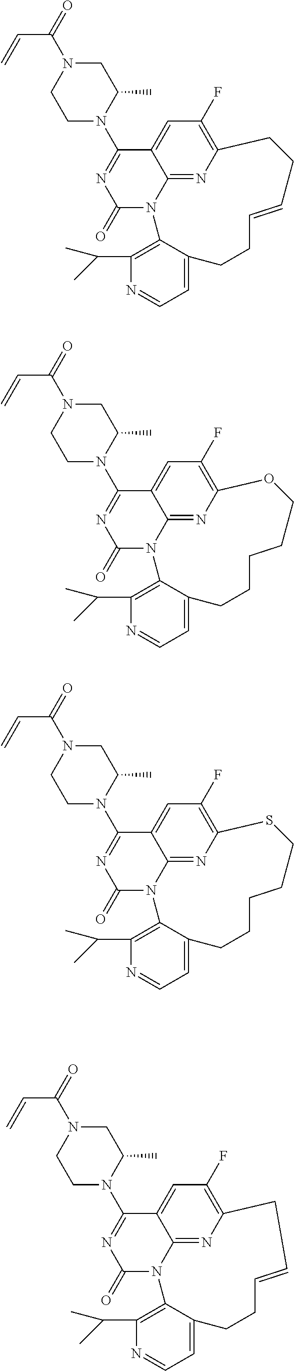 [SMILES: C=CC(=O)N1CCN(c2nc(=O)n3c4nc(c(F)cc24)CC/C=C/CCc2ccnc(C(C)C)c2-3)[C@@H](C)C1 ; C=CC(=O)N1CCN(c2nc(=O)n3c4nc(c(F)cc24)OCCCCCc2ccnc(C(C)C)c2-3)[C@@H](C)C1 ; C=CC(=O)N1CCN(c2nc(=O)n3c4nc(c(F)cc24)SCCCCCc2ccnc(C(C)C)c2-3)[C@@H](C)C1 ; C=CC(=O)N1CCN(c2nc(=O)n3c4nc(c(F)cc24)C/C=C/CCc2ccnc(C(C)C)c2-3)[C@@H](C)C1]

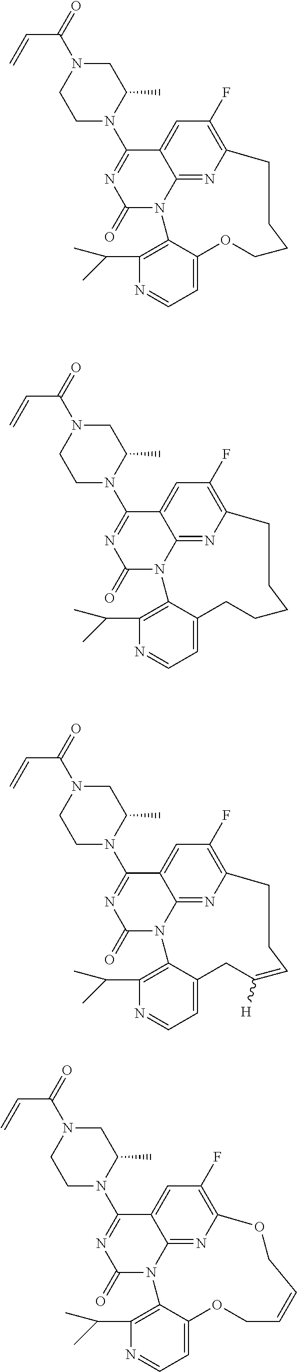 [SMILES: C=CC(=O)N1CCN(c2nc(=O)n3c4nc(c(F)cc24)CCCCOc2ccnc(C(C)C)c2-3)[C@@H](C)C1 ; C=CC(=O)N1CCN(c2nc(=O)n3c4nc(c(F)cc24)CCCCCc2ccnc(C(C)C)c2-3)[C@@H](C)C1 ; C=CC(=O)N1CCN(c2nc(=O)n3c4nc(c(F)cc24)CCC=CCc2ccnc(C(C)C)c2-3)[C@@H](C)C1 ; C=CC(=O)N1CCN(c2nc(=O)n3c4nc(c(F)cc24)OC/C=C\COc2ccnc(C(C)C)c2-3)[C@@H](C)C1]

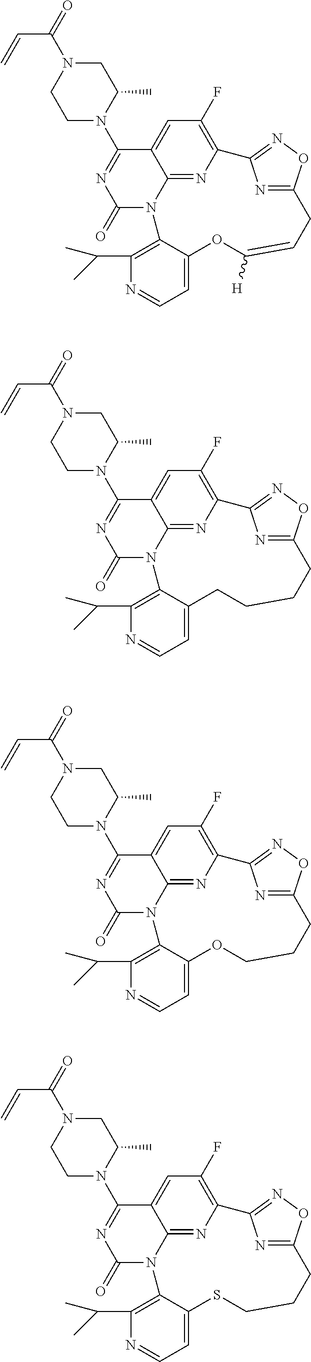 [SMILES: C=CC(=O)N1CCN(c2nc(=O)n3c4nc(c(F)cc24)-c2noc(n2)CC=COc2ccnc(C(C)C)c2-3)[C@@H](C)C1 ; C=CC(=O)N1CCN(c2nc(=O)n3c4nc(c(F)cc24)-c2noc(n2)CCCCc2ccnc(C(C)C)c2-3)[C@@H](C)C1 ; C=CC(=O)N1CCN(c2nc(=O)n3c4nc(c(F)cc24)-c2noc(n2)CCCOc2ccnc(C(C)C)c2-3)[C@@H](C)C1 ; C=CC(=O)N1CCN(c2nc(=O)n3c4nc(c(F)cc24)-c2noc(n2)CCCSc2ccnc(C(C)C)c2-3)[C@@H](C)C1]

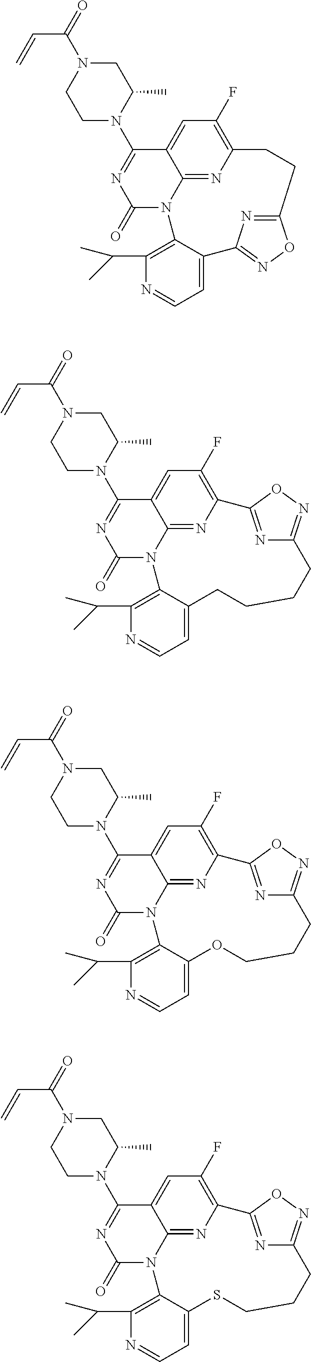 [SMILES: C=CC(=O)N1CCN(c2nc(=O)n3c4nc(c(F)cc24)CCc2nc(no2)-c2ccnc(C(C)C)c2-3)[C@@H](C)C1 ; C=CC(=O)N1CCN(c2nc(=O)n3c4nc(c(F)cc24)-c2nc(no2)CCCCc2ccnc(C(C)C)c2-3)[C@@H](C)C1 ; C=CC(=O)N1CCN(c2nc(=O)n3c4nc(c(F)cc24)-c2nc(no2)CCCOc2ccnc(C(C)C)c2-3)[C@@H](C)C1 ; C=CC(=O)N1CCN(c2nc(=O)n3c4nc(c(F)cc24)-c2nc(no2)CCCSc2ccnc(C(C)C)c2-3)[C@@H](C)C1]

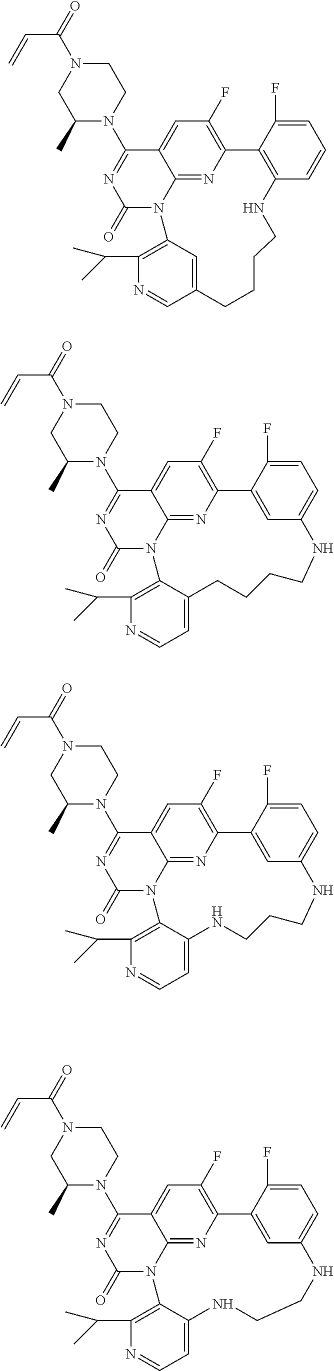 [SMILES: C=CC(=O)N1CCN(c2nc(=O)n3c4nc(c(F)cc24)-c2c(F)cccc2NCCCCc2cnc(C(C)C)c-3c2)[C@@H](C)C1 ; C=CC(=O)N1CCN(c2nc(=O)n3c4nc(c(F)cc24)-c2cc(ccc2F)NCCCCc2ccnc(C(C)C)c2-3)[C@@H](C)C1 ; C=CC(=O)N1CCN(c2nc(=O)n3c4nc(c(F)cc24)-c2cc(ccc2F)NCCCNc2ccnc(C(C)C)c2-3)[C@@H](C)C1 ; C=CC(=O)N1CCN(c2nc(=O)n3c4nc(c(F)cc24)-c2cc(ccc2F)NCCNc2ccnc(C(C)C)c2-3)[C@@H](C)C1]

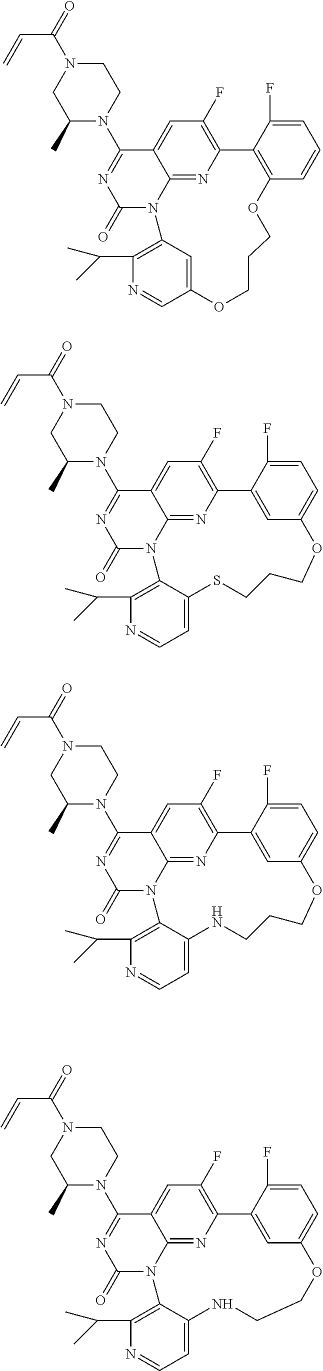 [SMILES: C=CC(=O)N1CCN(c2nc(=O)n3c4nc(c(F)cc24)-c2c(F)cccc2OCCCOc2cnc(C(C)C)c-3c2)[C@@H](C)C1 ; C=CC(=O)N1CCN(c2nc(=O)n3c4nc(c(F)cc24)-c2cc(ccc2F)OCCCSc2ccnc(C(C)C)c2-3)[C@@H](C)C1 ; C=CC(=O)N1CCN(c2nc(=O)n3c4nc(c(F)cc24)-c2cc(ccc2F)OCCCNc2ccnc(C(C)C)c2-3)[C@@H](C)C1 ; C=CC(=O)N1CCN(c2nc(=O)n3c4nc(c(F)cc24)-c2cc(ccc2F)OCCNc2ccnc(C(C)C)c2-3)[C@@H](C)C1]

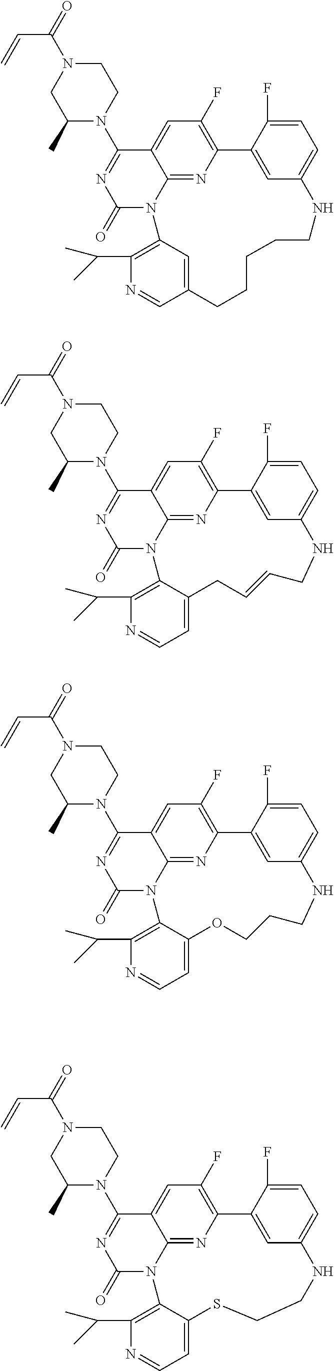 [SMILES: C=CC(=O)N1CCN(c2nc(=O)n3c4nc(c(F)cc24)-c2cc(ccc2F)NCCCCCc2cnc(C(C)C)c-3c2)[C@@H](C)C1 ; C=CC(=O)N1CCN(c2nc(=O)n3c4nc(c(F)cc24)-c2cc(ccc2F)NC/C=C/Cc2ccnc(C(C)C)c2-3)[C@@H](C)C1 ; C=CC(=O)N1CCN(c2nc(=O)n3c4nc(c(F)cc24)-c2cc(ccc2F)NCCCOc2ccnc(C(C)C)c2-3)[C@@H](C)C1 ; C=CC(=O)N1CCN(c2nc(=O)n3c4nc(c(F)cc24)-c2cc(ccc2F)NCCSc2ccnc(C(C)C)c2-3)[C@@H](C)C1]

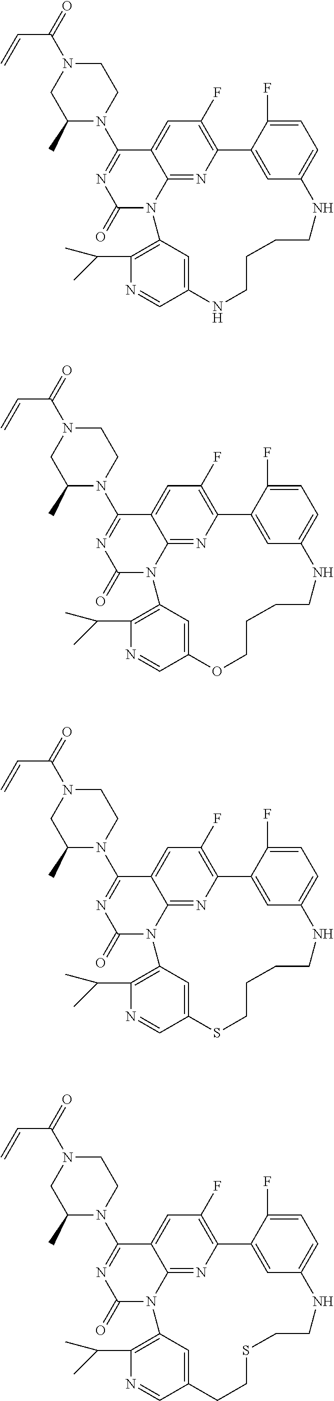 [SMILES: C=CC(=O)N1CCN(c2nc(=O)n3c4nc(c(F)cc24)-c2cc(ccc2F)NCCCCNc2cnc(C(C)C)c-3c2)[C@@H](C)C1 ; C=CC(=O)N1CCN(c2nc(=O)n3c4nc(c(F)cc24)-c2cc(ccc2F)NCCCCOc2cnc(C(C)C)c-3c2)[C@@H](C)C1 ; C=CC(=O)N1CCN(c2nc(=O)n3c4nc(c(F)cc24)-c2cc(ccc2F)NCCCCSc2cnc(C(C)C)c-3c2)[C@@H](C)C1 ; C=CC(=O)N1CCN(c2nc(=O)n3c4nc(c(F)cc24)-c2cc(ccc2F)NCCSCCc2cnc(C(C)C)c-3c2)[C@@H](C)C1]

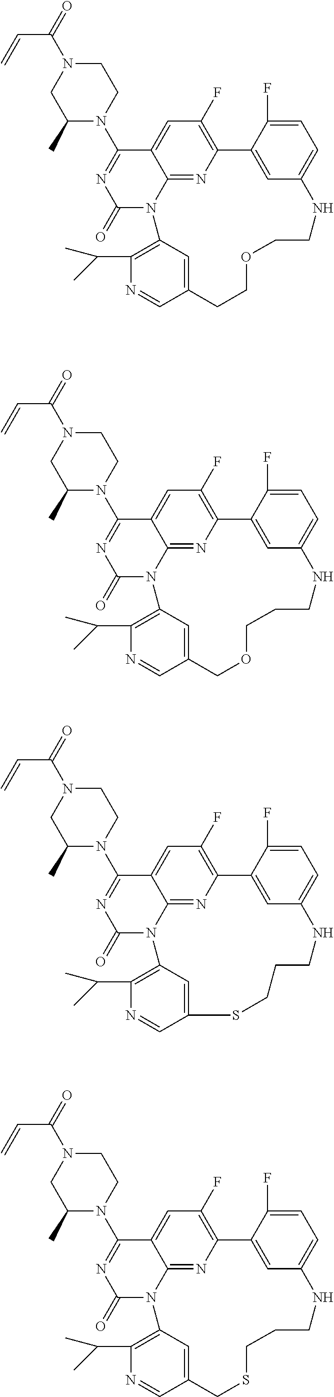 [SMILES: C=CC(=O)N1CCN(c2nc(=O)n3c4nc(c(F)cc24)-c2cc(ccc2F)NCCOCCc2cnc(C(C)C)c-3c2)[C@@H](C)C1 ; C=CC(=O)N1CCN(c2nc(=O)n3c4nc(c(F)cc24)-c2cc(ccc2F)NCCCOCc2cnc(C(C)C)c-3c2)[C@@H](C)C1 ; C=CC(=O)N1CCN(c2nc(=O)n3c4nc(c(F)cc24)-c2cc(ccc2F)NCCCSc2cnc(C(C)C)c-3c2)[C@@H](C)C1 ; C=CC(=O)N1CCN(c2nc(=O)n3c4nc(c(F)cc24)-c2cc(ccc2F)NCCCSCc2cnc(C(C)C)c-3c2)[C@@H](C)C1]

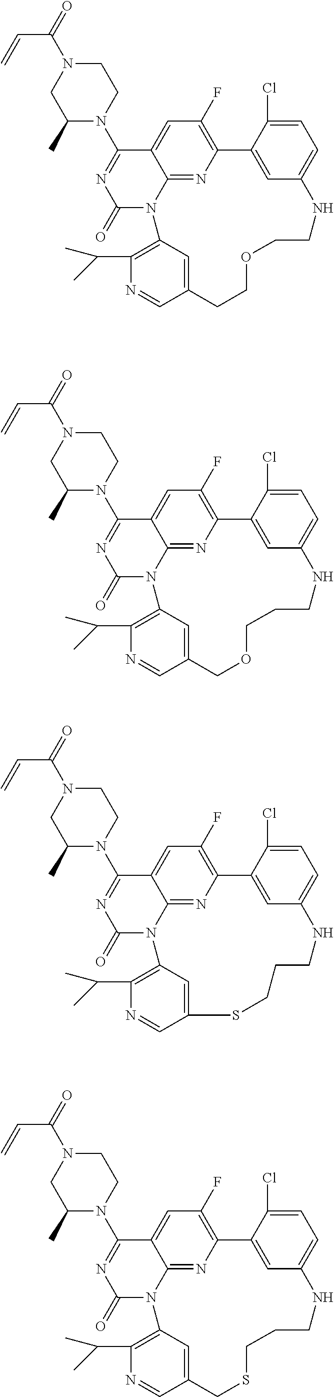 [SMILES: C=CC(=O)N1CCN(c2nc(=O)n3c4nc(c(F)cc24)-c2cc(ccc2Cl)NCCOCCc2cnc(C(C)C)c-3c2)[C@@H](C)C1 ; C=CC(=O)N1CCN(c2nc(=O)n3c4nc(c(F)cc24)-c2cc(ccc2Cl)NCCCOCc2cnc(C(C)C)c-3c2)[C@@H](C)C1 ; C=CC(=O)N1CCN(c2nc(=O)n3c4nc(c(F)cc24)-c2cc(ccc2Cl)NCCCSc2cnc(C(C)C)c-3c2)[C@@H](C)C1 ; C=CC(=O)N1CCN(c2nc(=O)n3c4nc(c(F)cc24)-c2cc(ccc2Cl)NCCCSCc2cnc(C(C)C)c-3c2)[C@@H](C)C1]

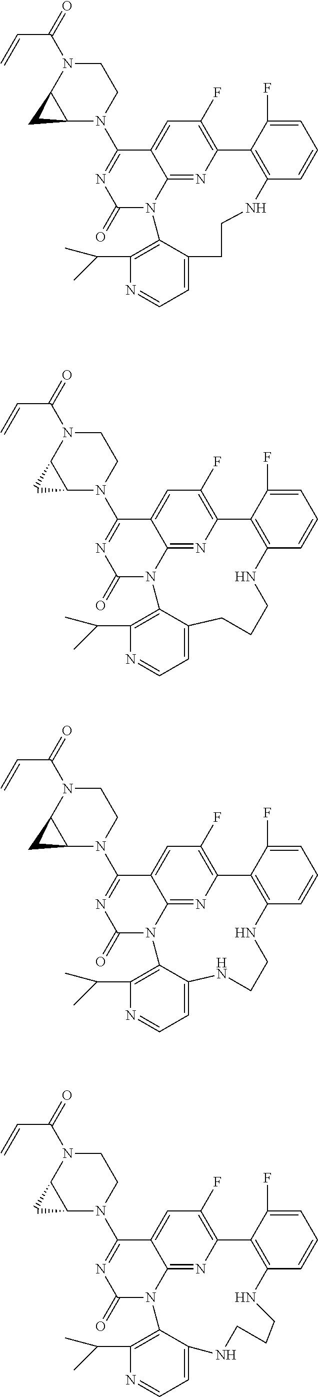 [SMILES: C=CC(=O)N1CCN(c2nc(=O)n3c4nc(c(F)cc24)-c2c(F)cccc2NCCc2ccnc(C(C)C)c2-3)[C@H]2C[C@H]21 ; C=CC(=O)N1CCN(c2nc(=O)n3c4nc(c(F)cc24)-c2c(F)cccc2NCCCc2ccnc(C(C)C)c2-3)[C@@H]2C[C@@H]21 ; C=CC(=O)N1CCN(c2nc(=O)n3c4nc(c(F)cc24)-c2c(F)cccc2NCCNc2ccnc(C(C)C)c2-3)[C@H]2C[C@H]21 ; C=CC(=O)N1CCN(c2nc(=O)n3c4nc(c(F)cc24)-c2c(F)cccc2NCCCNc2ccnc(C(C)C)c2-3)[C@@H]2C[C@@H]21]

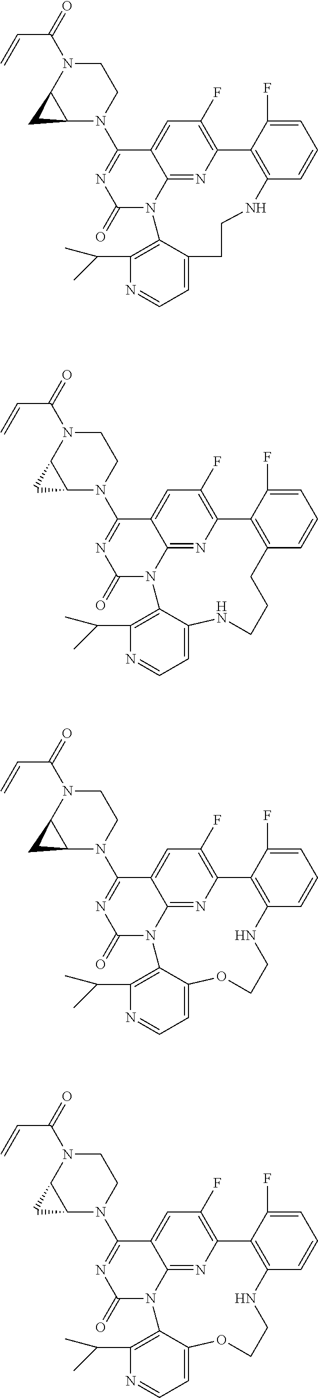 [SMILES: C=CC(=O)N1CCN(c2nc(=O)n3c4nc(c(F)cc24)-c2c(F)cccc2NCCc2ccnc(C(C)C)c2-3)[C@H]2C[C@H]21 ; C=CC(=O)N1CCN(c2nc(=O)n3c4nc(c(F)cc24)-c2c(F)cccc2CCCNc2ccnc(C(C)C)c2-3)[C@@H]2C[C@@H]21 ; C=CC(=O)N1CCN(c2nc(=O)n3c4nc(c(F)cc24)-c2c(F)cccc2NCCOc2ccnc(C(C)C)c2-3)[C@H]2C[C@H]21 ; C=CC(=O)N1CCN(c2nc(=O)n3c4nc(c(F)cc24)-c2c(F)cccc2NCCOc2ccnc(C(C)C)c2-3)[C@@H]2C[C@@H]21]

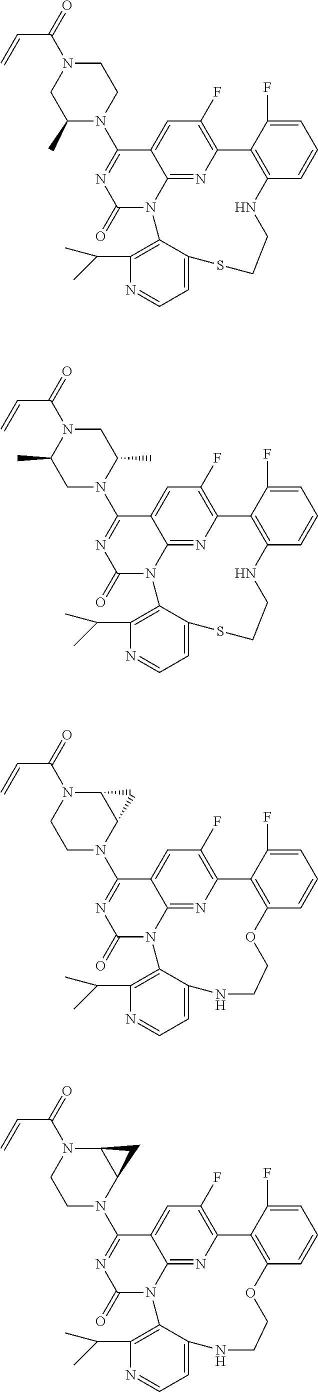 [SMILES: C=CC(=O)N1CCN(c2nc(=O)n3c4nc(c(F)cc24)-c2c(F)cccc2NCCSc2ccnc(C(C)C)c2-3)[C@@H](C)C1 ; C=CC(=O)N1C[C@H](C)N(c2nc(=O)n3c4nc(c(F)cc24)-c2c(F)cccc2NCCSc2ccnc(C(C)C)c2-3)C[C@H]1C ; C=CC(=O)N1CCN(c2nc(=O)n3c4nc(c(F)cc24)-c2c(F)cccc2OCCNc2ccnc(C(C)C)c2-3)[C@H]2C[C@H]21 ; C=CC(=O)N1CCN(c2nc(=O)n3c4nc(c(F)cc24)-c2c(F)cccc2OCCNc2ccnc(C(C)C)c2-3)[C@@H]2C[C@@H]21]

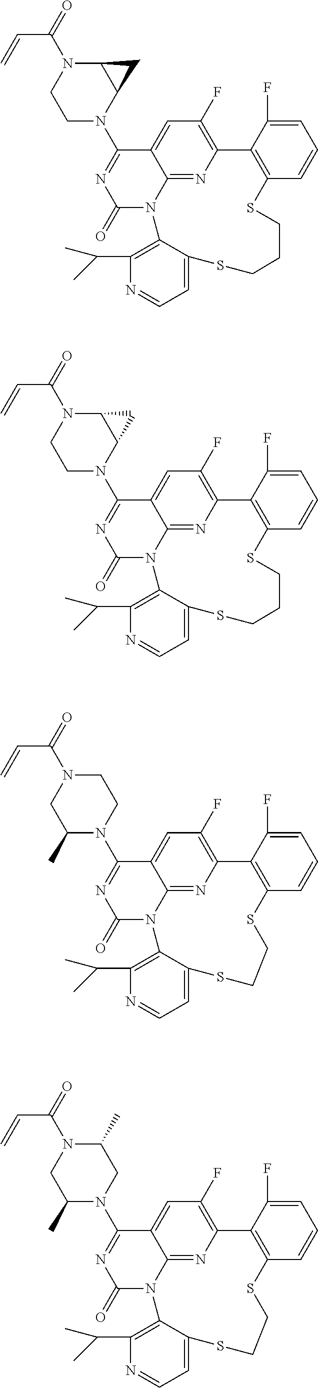 [SMILES: C=CC(=O)N1CCN(c2nc(=O)n3c4nc(c(F)cc24)-c2c(F)cccc2SCCCSc2ccnc(C(C)C)c2-3)[C@@H]2C[C@@H]21 ; C=CC(=O)N1CCN(c2nc(=O)n3c4nc(c(F)cc24)-c2c(F)cccc2SCCCSc2ccnc(C(C)C)c2-3)[C@H]2C[C@H]21 ; C=CC(=O)N1CCN(c2nc(=O)n3c4nc(c(F)cc24)-c2c(F)cccc2SCCSc2ccnc(C(C)C)c2-3)[C@@H](C)C1 ; C=CC(=O)N1C[C@H](C)N(c2nc(=O)n3c4nc(c(F)cc24)-c2c(F)cccc2SCCSc2ccnc(C(C)C)c2-3)C[C@H]1C]

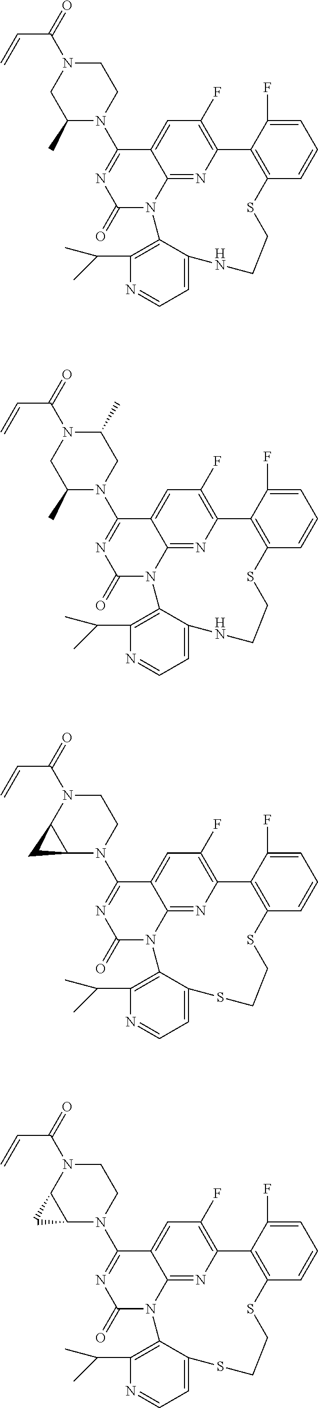 [SMILES: C=CC(=O)N1CCN(c2nc(=O)n3c4nc(c(F)cc24)-c2c(F)cccc2SCCNc2ccnc(C(C)C)c2-3)[C@@H](C)C1 ; C=CC(=O)N1C[C@H](C)N(c2nc(=O)n3c4nc(c(F)cc24)-c2c(F)cccc2SCCNc2ccnc(C(C)C)c2-3)C[C@H]1C ; C=CC(=O)N1CCN(c2nc(=O)n3c4nc(c(F)cc24)-c2c(F)cccc2SCCSc2ccnc(C(C)C)c2-3)[C@H]2C[C@H]21 ; C=CC(=O)N1CCN(c2nc(=O)n3c4nc(c(F)cc24)-c2c(F)cccc2SCCSc2ccnc(C(C)C)c2-3)[C@@H]2C[C@@H]21]

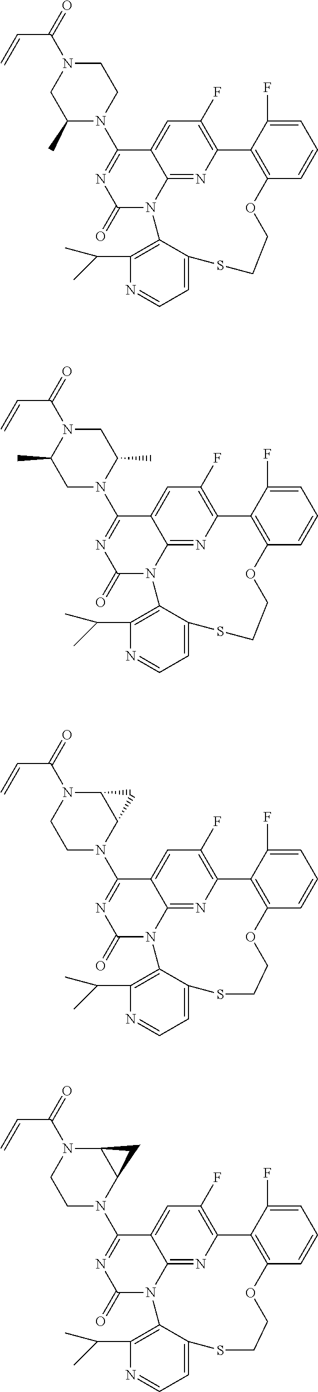 [SMILES: C=CC(=O)N1CCN(c2nc(=O)n3c4nc(c(F)cc24)-c2c(F)cccc2OCCSc2ccnc(C(C)C)c2-3)[C@@H](C)C1 ; C=CC(=O)N1C[C@H](C)N(c2nc(=O)n3c4nc(c(F)cc24)-c2c(F)cccc2OCCSc2ccnc(C(C)C)c2-3)C[C@H]1C ; C=CC(=O)N1CCN(c2nc(=O)n3c4nc(c(F)cc24)-c2c(F)cccc2OCCSc2ccnc(C(C)C)c2-3)[C@H]2C[C@H]21 ; C=CC(=O)N1CCN(c2nc(=O)n3c4nc(c(F)cc24)-c2c(F)cccc2OCCSc2ccnc(C(C)C)c2-3)[C@@H]2C[C@@H]21]

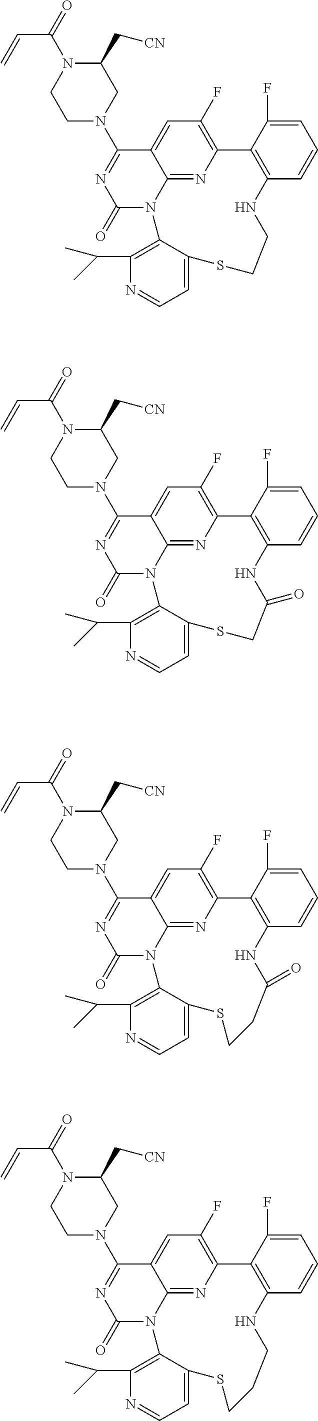 [SMILES: C=CC(=O)N1CCN(c2nc(=O)n3c4nc(c(F)cc24)-c2c(F)cccc2NCCSc2ccnc(C(C)C)c2-3)C[C@@H]1CC#N ; C=CC(=O)N1CCN(c2nc(=O)n3c4nc(c(F)cc24)-c2c(F)cccc2NC(=O)CSc2ccnc(C(C)C)c2-3)C[C@@H]1CC#N ; C=CC(=O)N1CCN(c2nc(=O)n3c4nc(c(F)cc24)-c2c(F)cccc2NC(=O)CCSc2ccnc(C(C)C)c2-3)C[C@@H]1CC#N ; C=CC(=O)N1CCN(c2nc(=O)n3c4nc(c(F)cc24)-c2c(F)cccc2NCCCSc2ccnc(C(C)C)c2-3)C[C@@H]1CC#N]

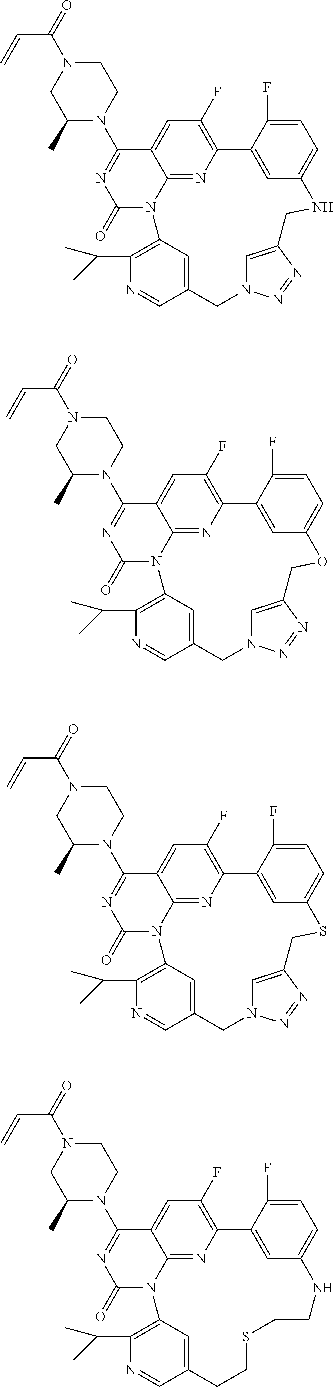 [SMILES: C=CC(=O)N1CCN(c2nc(=O)n3c4nc(c(F)cc24)-c2cc(ccc2F)NCc2cn(nn2)Cc2cnc(C(C)C)c-3c2)[C@@H](C)C1 ; C=CC(=O)N1CCN(c2nc(=O)n3c4nc(c(F)cc24)-c2cc(ccc2F)OCc2cn(nn2)Cc2cnc(C(C)C)c-3c2)[C@@H](C)C1 ; C=CC(=O)N1CCN(c2nc(=O)n3c4nc(c(F)cc24)-c2cc(ccc2F)SCc2cn(nn2)Cc2cnc(C(C)C)c-3c2)[C@@H](C)C1 ; C=CC(=O)N1CCN(c2nc(=O)n3c4nc(c(F)cc24)-c2cc(ccc2F)NCCSCCc2cnc(C(C)C)c-3c2)[C@@H](C)C1]

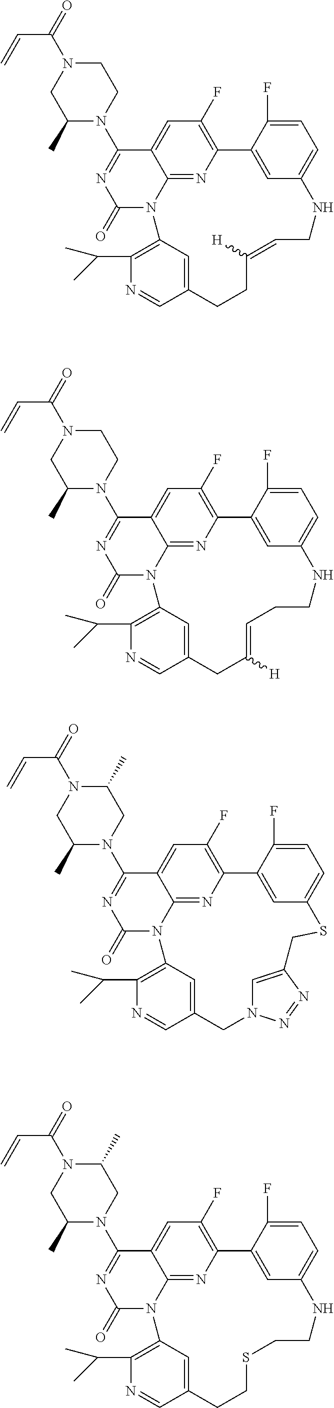 [SMILES: C=CC(=O)N1CCN(c2nc(=O)n3c4nc(c(F)cc24)-c2cc(ccc2F)NCC=CCCc2cnc(C(C)C)c-3c2)[C@@H](C)C1 ; C=CC(=O)N1CCN(c2nc(=O)n3c4nc(c(F)cc24)-c2cc(ccc2F)NCCC=CCc2cnc(C(C)C)c-3c2)[C@@H](C)C1 ; C=CC(=O)N1C[C@H](C)N(c2nc(=O)n3c4nc(c(F)cc24)-c2cc(ccc2F)SCc2cn(nn2)Cc2cnc(C(C)C)c-3c2)C[C@H]1C ; C=CC(=O)N1C[C@H](C)N(c2nc(=O)n3c4nc(c(F)cc24)-c2cc(ccc2F)NCCSCCc2cnc(C(C)C)c-3c2)C[C@H]1C]

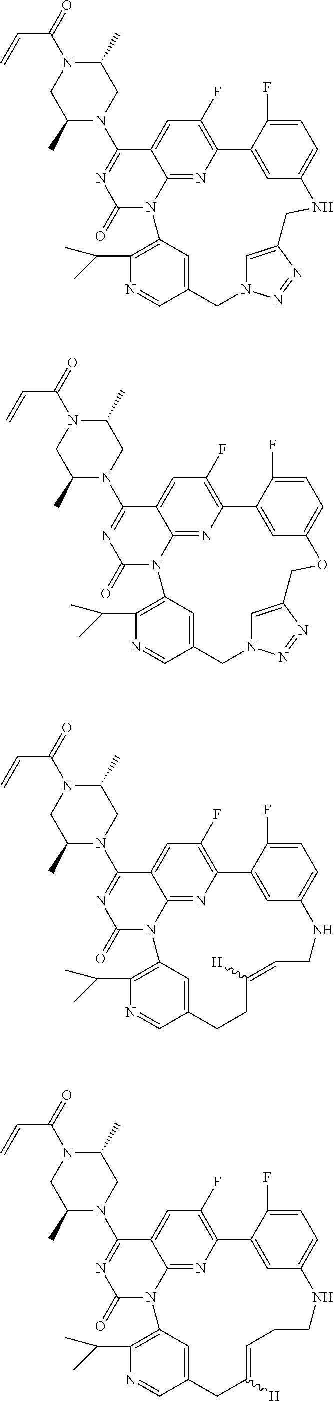 [SMILES: C=CC(=O)N1C[C@H](C)N(c2nc(=O)n3c4nc(c(F)cc24)-c2cc(ccc2F)NCc2cn(nn2)Cc2cnc(C(C)C)c-3c2)C[C@H]1C ; C=CC(=O)N1C[C@H](C)N(c2nc(=O)n3c4nc(c(F)cc24)-c2cc(ccc2F)OCc2cn(nn2)Cc2cnc(C(C)C)c-3c2)C[C@H]1C ; C=CC(=O)N1C[C@H](C)N(c2nc(=O)n3c4nc(c(F)cc24)-c2cc(ccc2F)NCC=CCCc2cnc(C(C)C)c-3c2)C[C@H]1C ; C=CC(=O)N1C[C@H](C)N(c2nc(=O)n3c4nc(c(F)cc24)-c2cc(ccc2F)NCCC=CCc2cnc(C(C)C)c-3c2)C[C@H]1C]

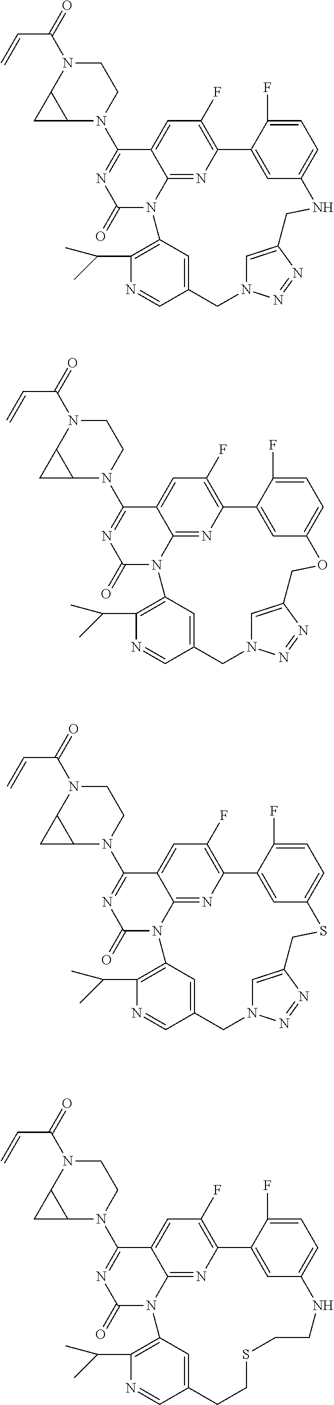 [SMILES: C=CC(=O)N1CCN(c2nc(=O)n3c4nc(c(F)cc24)-c2cc(ccc2F)NCc2cn(nn2)Cc2cnc(C(C)C)c-3c2)C2CC21 ; C=CC(=O)N1CCN(c2nc(=O)n3c4nc(c(F)cc24)-c2cc(ccc2F)OCc2cn(nn2)Cc2cnc(C(C)C)c-3c2)C2CC21 ; C=CC(=O)N1CCN(c2nc(=O)n3c4nc(c(F)cc24)-c2cc(ccc2F)SCc2cn(nn2)Cc2cnc(C(C)C)c-3c2)C2CC21 ; C=CC(=O)N1CCN(c2nc(=O)n3c4nc(c(F)cc24)-c2cc(ccc2F)NCCSCCc2cnc(C(C)C)c-3c2)C2CC21]

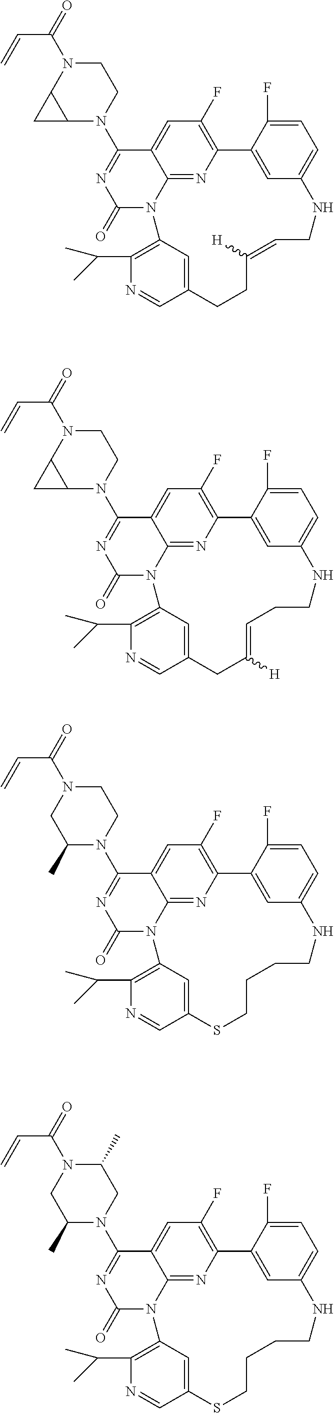 [SMILES: C=CC(=O)N1CCN(c2nc(=O)n3c4nc(c(F)cc24)-c2cc(ccc2F)NCC=CCCc2cnc(C(C)C)c-3c2)C2CC21 ; C=CC(=O)N1CCN(c2nc(=O)n3c4nc(c(F)cc24)-c2cc(ccc2F)NCCC=CCc2cnc(C(C)C)c-3c2)C2CC21 ; C=CC(=O)N1CCN(c2nc(=O)n3c4nc(c(F)cc24)-c2cc(ccc2F)NCCCCSc2cnc(C(C)C)c-3c2)[C@@H](C)C1 ; C=CC(=O)N1C[C@H](C)N(c2nc(=O)n3c4nc(c(F)cc24)-c2cc(ccc2F)NCCCCSc2cnc(C(C)C)c-3c2)C[C@H]1C]

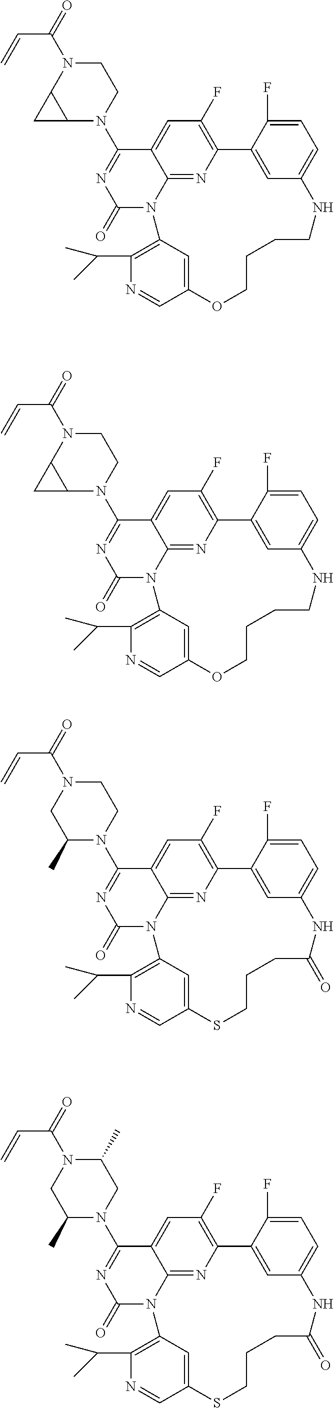 [SMILES: C=CC(=O)N1CCN(c2nc(=O)n3c4nc(c(F)cc24)-c2cc(ccc2F)NCCCCOc2cnc(C(C)C)c-3c2)C2CC21 ; C=CC(=O)N1CCN(c2nc(=O)n3c4nc(c(F)cc24)-c2cc(ccc2F)NCCCCOc2cnc(C(C)C)c-3c2)C2CC21 ; C=CC(=O)N1CCN(c2nc(=O)n3c4nc(c(F)cc24)-c2cc(ccc2F)NC(=O)CCCSc2cnc(C(C)C)c-3c2)[C@@H](C)C1 ; C=CC(=O)N1C[C@H](C)N(c2nc(=O)n3c4nc(c(F)cc24)-c2cc(ccc2F)NC(=O)CCCSc2cnc(C(C)C)c-3c2)C[C@H]1C]

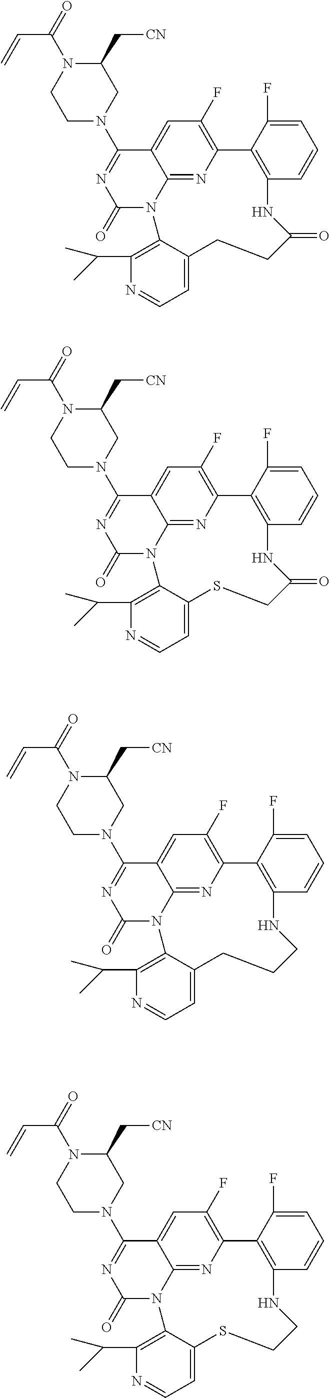 [SMILES: C=CC(=O)N1CCN(c2nc(=O)n3c4nc(c(F)cc24)-c2c(F)cccc2NC(=O)CCc2ccnc(C(C)C)c2-3)C[C@@H]1CC#N ; C=CC(=O)N1CCN(c2nc(=O)n3c4nc(c(F)cc24)-c2c(F)cccc2NC(=O)CSc2ccnc(C(C)C)c2-3)C[C@@H]1CC#N ; C=CC(=O)N1CCN(c2nc(=O)n3c4nc(c(F)cc24)-c2c(F)cccc2NCCCc2ccnc(C(C)C)c2-3)C[C@@H]1CC#N ; C=CC(=O)N1CCN(c2nc(=O)n3c4nc(c(F)cc24)-c2c(F)cccc2NCCSc2ccnc(C(C)C)c2-3)C[C@@H]1CC#N]

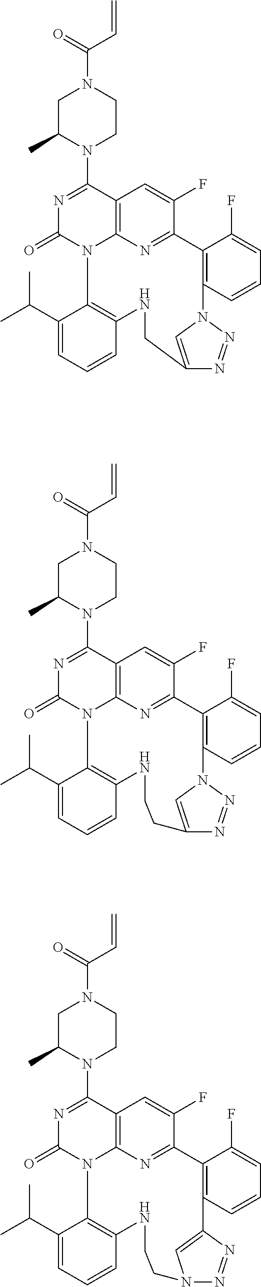 [SMILES: C=CC(=O)N1CCN(c2nc(=O)n3c4nc(c(F)cc24)-c2c(F)cccc2-n2cc(nn2)CNc2cccc(C(C)C)c2-3)[C@@H](C)C1 ; C=CC(=O)N1CCN(c2nc(=O)n3c4nc(c(F)cc24)-c2c(F)cccc2-n2cc(nn2)CCNc2cccc(C(C)C)c2-3)[C@@H](C)C1 ; C=CC(=O)N1CCN(c2nc(=O)n3c4nc(c(F)cc24)-c2c(F)cccc2-c2cn(nn2)CCNc2cccc(C(C)C)c2-3)[C@@H](C)C1]

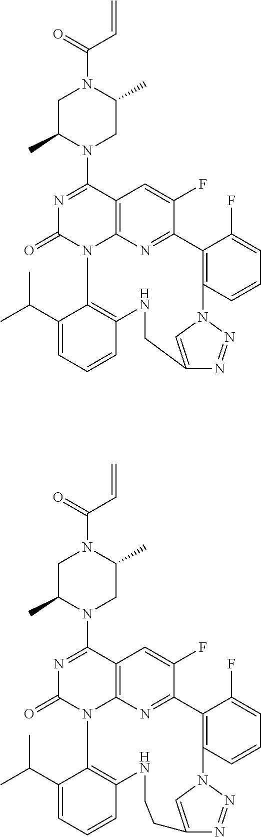 [SMILES: C=CC(=O)N1C[C@H](C)N(c2nc(=O)n3c4nc(c(F)cc24)-c2c(F)cccc2-n2cc(nn2)CNc2cccc(C(C)C)c2-3)C[C@H]1C ; C=CC(=O)N1C[C@H](C)N(c2nc(=O)n3c4nc(c(F)cc24)-c2c(F)cccc2-n2cc(nn2)CCNc2cccc(C(C)C)c2-3)C[C@H]1C]

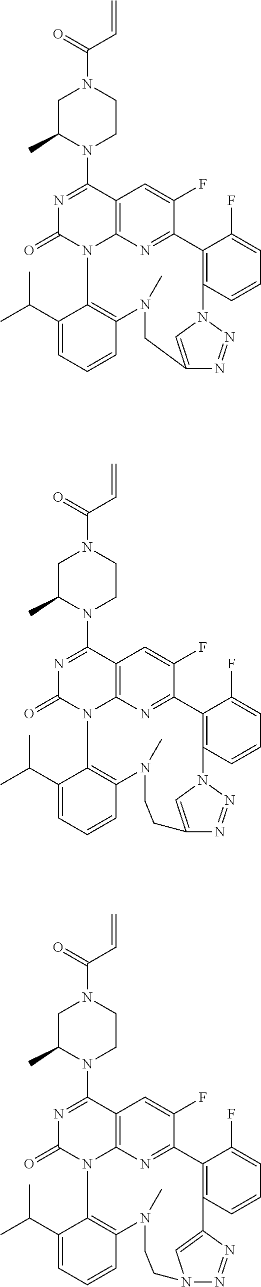 [SMILES: C=CC(=O)N1CCN(c2nc(=O)n3c4nc(c(F)cc24)-c2c(F)cccc2-n2cc(nn2)CN(C)c2cccc(C(C)C)c2-3)[C@@H](C)C1 ; C=CC(=O)N1CCN(c2nc(=O)n3c4nc(c(F)cc24)-c2c(F)cccc2-n2cc(nn2)CCN(C)c2cccc(C(C)C)c2-3)[C@@H](C)C1 ; C=CC(=O)N1CCN(c2nc(=O)n3c4nc(c(F)cc24)-c2c(F)cccc2-c2cn(nn2)CCN(C)c2cccc(C(C)C)c2-3)[C@@H](C)C1]

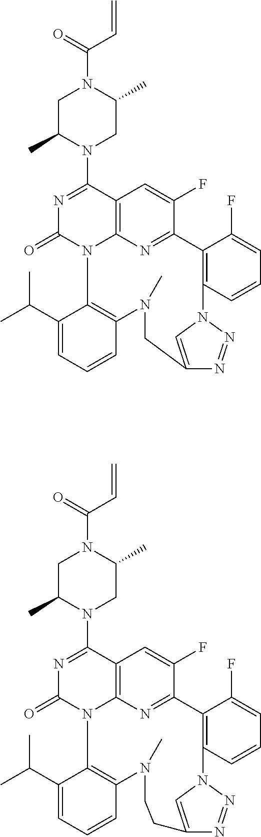 [SMILES: C=CC(=O)N1C[C@H](C)N(c2nc(=O)n3c4nc(c(F)cc24)-c2c(F)cccc2-n2cc(nn2)CN(C)c2cccc(C(C)C)c2-3)C[C@H]1C ; C=CC(=O)N1C[C@H](C)N(c2nc(=O)n3c4nc(c(F)cc24)-c2c(F)cccc2-n2cc(nn2)CCN(C)c2cccc(C(C)C)c2-3)C[C@H]1C]

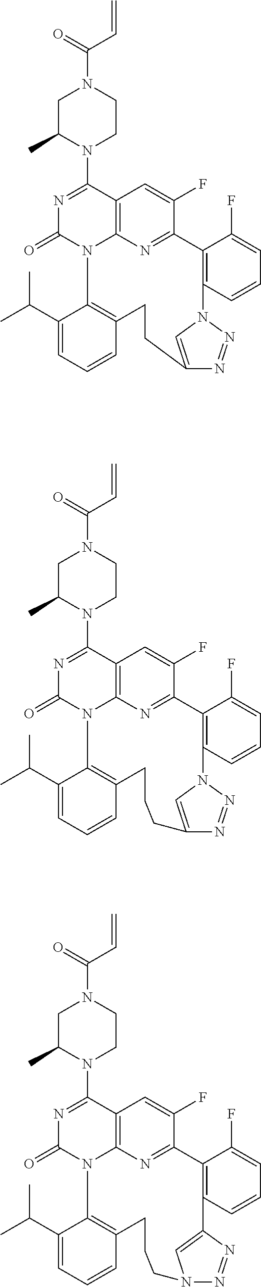 [SMILES: C=CC(=O)N1CCN(c2nc(=O)n3c4nc(c(F)cc24)-c2c(F)cccc2-n2cc(nn2)CCc2cccc(C(C)C)c2-3)[C@@H](C)C1 ; C=CC(=O)N1CCN(c2nc(=O)n3c4nc(c(F)cc24)-c2c(F)cccc2-n2cc(nn2)CCCc2cccc(C(C)C)c2-3)[C@@H](C)C1 ; C=CC(=O)N1CCN(c2nc(=O)n3c4nc(c(F)cc24)-c2c(F)cccc2-c2cn(nn2)CCCc2cccc(C(C)C)c2-3)[C@@H](C)C1]

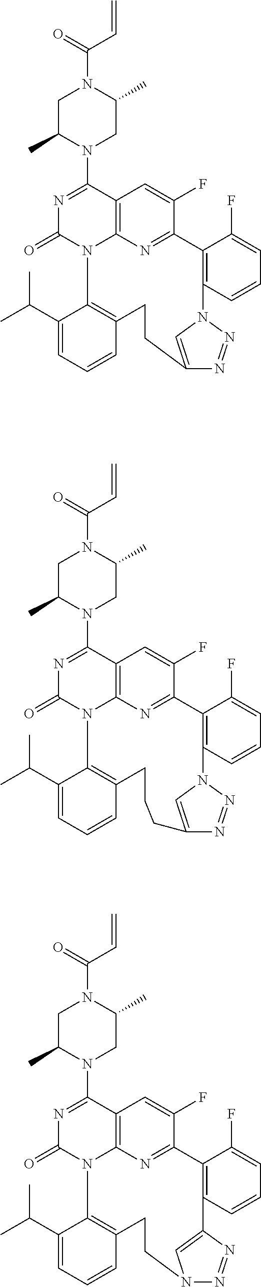 [SMILES: C=CC(=O)N1C[C@H](C)N(c2nc(=O)n3c4nc(c(F)cc24)-c2c(F)cccc2-n2cc(nn2)CCc2cccc(C(C)C)c2-3)C[C@H]1C ; C=CC(=O)N1C[C@H](C)N(c2nc(=O)n3c4nc(c(F)cc24)-c2c(F)cccc2-n2cc(nn2)CCCc2cccc(C(C)C)c2-3)C[C@H]1C ; C=CC(=O)N1C[C@H](C)N(c2nc(=O)n3c4nc(c(F)cc24)-c2c(F)cccc2-c2cn(nn2)CCc2cccc(C(C)C)c2-3)C[C@H]1C]

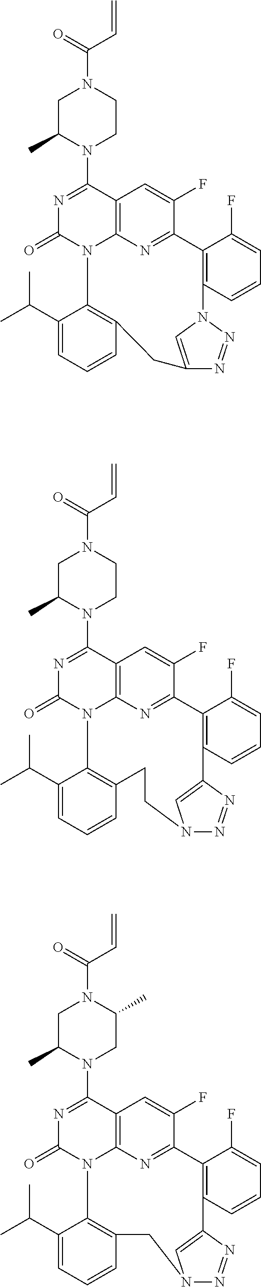 [SMILES: C=CC(=O)N1CCN(c2nc(=O)n3c4nc(c(F)cc24)-c2c(F)cccc2-n2cc(nn2)Cc2cccc(C(C)C)c2-3)[C@@H](C)C1 ; C=CC(=O)N1CCN(c2nc(=O)n3c4nc(c(F)cc24)-c2c(F)cccc2-c2cn(nn2)CCc2cccc(C(C)C)c2-3)[C@@H](C)C1 ; C=CC(=O)N1C[C@H](C)N(c2nc(=O)n3c4nc(c(F)cc24)-c2c(F)cccc2-c2cn(nn2)Cc2cccc(C(C)C)c2-3)C[C@H]1C]

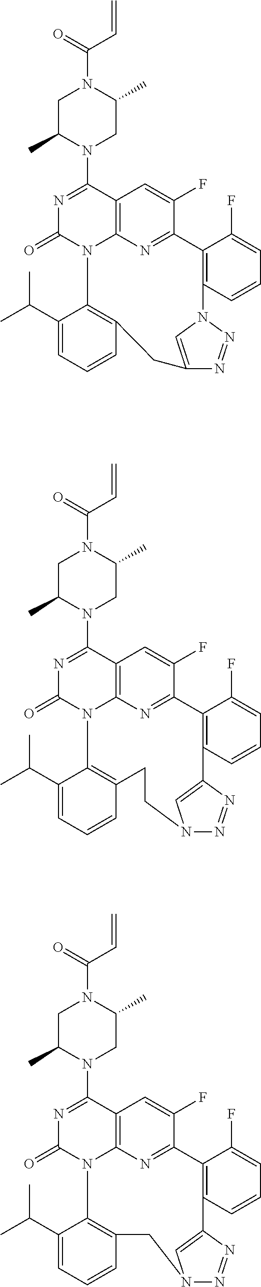 [SMILES: C=CC(=O)N1C[C@H](C)N(c2nc(=O)n3c4nc(c(F)cc24)-c2c(F)cccc2-n2cc(nn2)Cc2cccc(C(C)C)c2-3)C[C@H]1C ; C=CC(=O)N1C[C@H](C)N(c2nc(=O)n3c4nc(c(F)cc24)-c2c(F)cccc2-c2cn(nn2)CCc2cccc(C(C)C)c2-3)C[C@H]1C ; C=CC(=O)N1C[C@H](C)N(c2nc(=O)n3c4nc(c(F)cc24)-c2c(F)cccc2-c2cn(nn2)Cc2cccc(C(C)C)c2-3)C[C@H]1C]

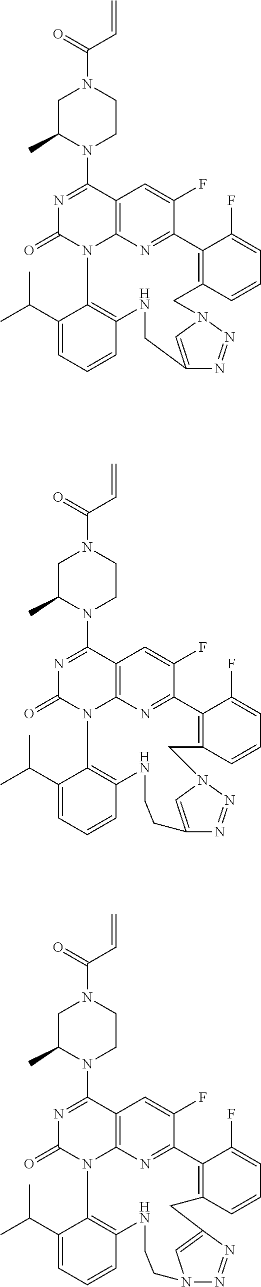 [SMILES: C=CC(=O)N1CCN(c2nc(=O)n3c4nc(c(F)cc24)-c2c(F)cccc2Cn2cc(nn2)CNc2cccc(C(C)C)c2-3)[C@@H](C)C1 ; C=CC(=O)N1CCN(c2nc(=O)n3c4nc(c(F)cc24)-c2c(F)cccc2Cn2cc(nn2)CCNc2cccc(C(C)C)c2-3)[C@@H](C)C1 ; C=CC(=O)N1CCN(c2nc(=O)n3c4nc(c(F)cc24)-c2c(F)cccc2Cc2cn(nn2)CCNc2cccc(C(C)C)c2-3)[C@@H](C)C1]

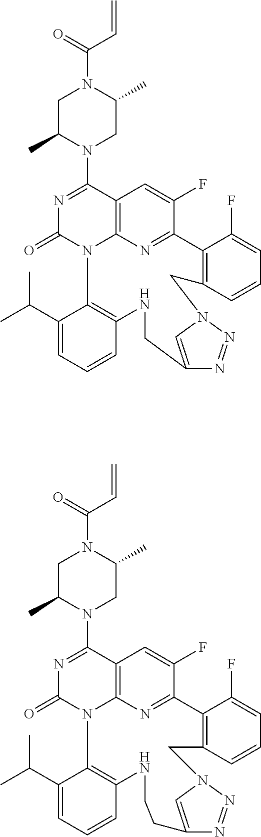 [SMILES: C=CC(=O)N1C[C@H](C)N(c2nc(=O)n3c4nc(c(F)cc24)-c2c(F)cccc2Cn2cc(nn2)CNc2cccc(C(C)C)c2-3)C[C@H]1C ; C=CC(=O)N1C[C@H](C)N(c2nc(=O)n3c4nc(c(F)cc24)-c2c(F)cccc2Cn2cc(nn2)CCNc2cccc(C(C)C)c2-3)C[C@H]1C]

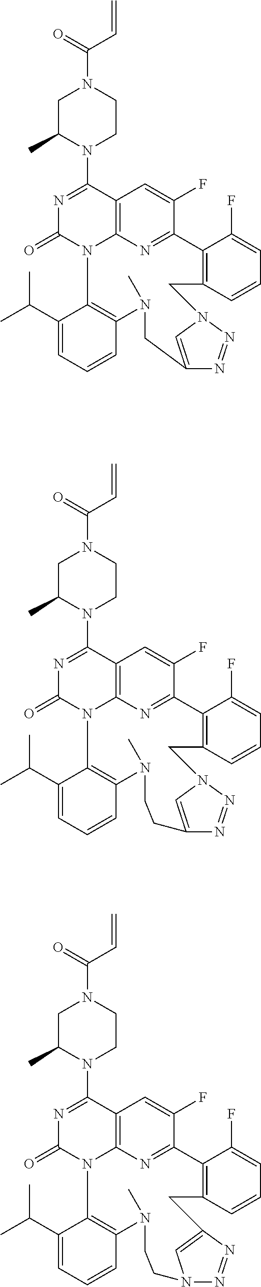 [SMILES: C=CC(=O)N1CCN(c2nc(=O)n3c4nc(c(F)cc24)-c2c(F)cccc2Cn2cc(nn2)CN(C)c2cccc(C(C)C)c2-3)[C@@H](C)C1 ; C=CC(=O)N1CCN(c2nc(=O)n3c4nc(c(F)cc24)-c2c(F)cccc2Cn2cc(nn2)CCN(C)c2cccc(C(C)C)c2-3)[C@@H](C)C1 ; C=CC(=O)N1CCN(c2nc(=O)n3c4nc(c(F)cc24)-c2c(F)cccc2Cc2cn(nn2)CCN(C)c2cccc(C(C)C)c2-3)[C@@H](C)C1]

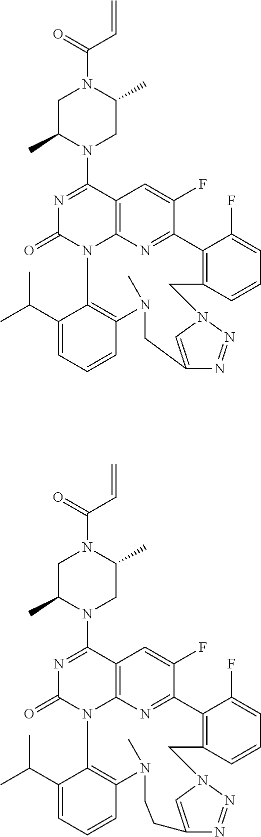 [SMILES: C=CC(=O)N1C[C@H](C)N(c2nc(=O)n3c4nc(c(F)cc24)-c2c(F)cccc2Cn2cc(nn2)CN(C)c2cccc(C(C)C)c2-3)C[C@H]1C ; C=CC(=O)N1C[C@H](C)N(c2nc(=O)n3c4nc(c(F)cc24)-c2c(F)cccc2Cn2cc(nn2)CCN(C)c2cccc(C(C)C)c2-3)C[C@H]1C]

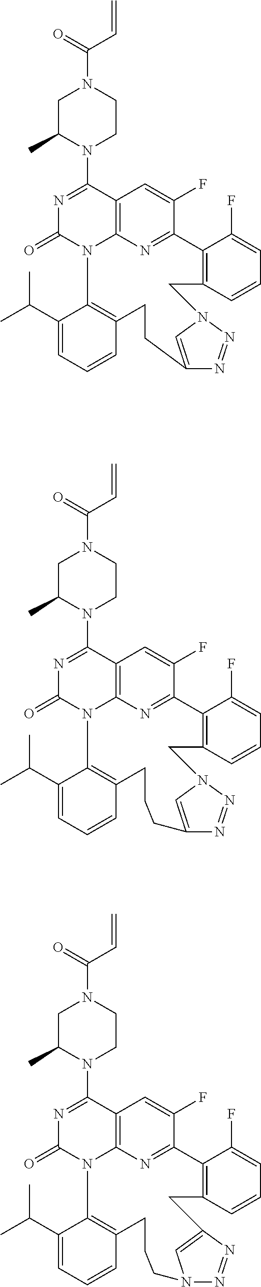 [SMILES: C=CC(=O)N1CCN(c2nc(=O)n3c4nc(c(F)cc24)-c2c(F)cccc2Cn2cc(nn2)CCc2cccc(C(C)C)c2-3)[C@@H](C)C1 ; C=CC(=O)N1CCN(c2nc(=O)n3c4nc(c(F)cc24)-c2c(F)cccc2Cn2cc(nn2)CCCc2cccc(C(C)C)c2-3)[C@@H](C)C1 ; C=CC(=O)N1CCN(c2nc(=O)n3c4nc(c(F)cc24)-c2c(F)cccc2Cc2cn(nn2)CCCc2cccc(C(C)C)c2-3)[C@@H](C)C1]

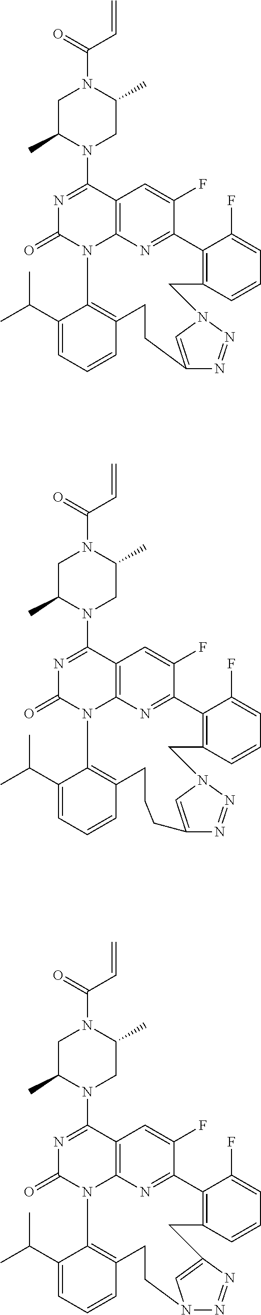 [SMILES: C=CC(=O)N1C[C@H](C)N(c2nc(=O)n3c4nc(c(F)cc24)-c2c(F)cccc2Cn2cc(nn2)CCc2cccc(C(C)C)c2-3)C[C@H]1C ; C=CC(=O)N1C[C@H](C)N(c2nc(=O)n3c4nc(c(F)cc24)-c2c(F)cccc2Cn2cc(nn2)CCCc2cccc(C(C)C)c2-3)C[C@H]1C ; C=CC(=O)N1C[C@H](C)N(c2nc(=O)n3c4nc(c(F)cc24)-c2c(F)cccc2Cc2cn(nn2)CCc2cccc(C(C)C)c2-3)C[C@H]1C]

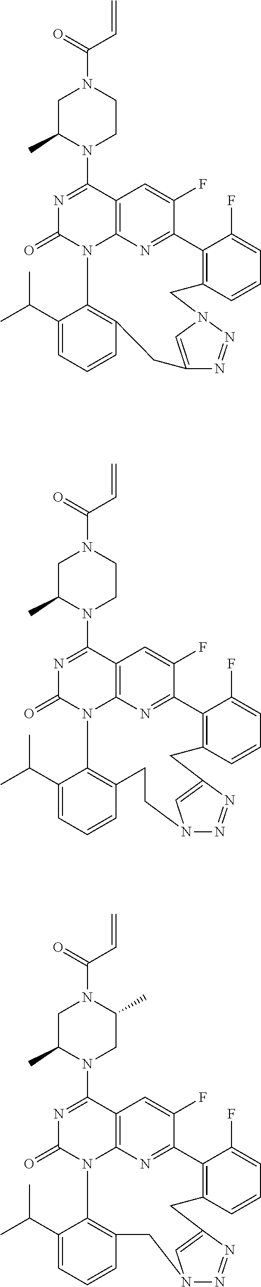 [SMILES: C=CC(=O)N1CCN(c2nc(=O)n3c4nc(c(F)cc24)-c2c(F)cccc2Cn2cc(nn2)Cc2cccc(C(C)C)c2-3)[C@@H](C)C1 ; C=CC(=O)N1CCN(c2nc(=O)n3c4nc(c(F)cc24)-c2c(F)cccc2Cc2cn(nn2)CCc2cccc(C(C)C)c2-3)[C@@H](C)C1 ; C=CC(=O)N1C[C@H](C)N(c2nc(=O)n3c4nc(c(F)cc24)-c2c(F)cccc2Cc2cn(nn2)Cc2cccc(C(C)C)c2-3)C[C@H]1C]

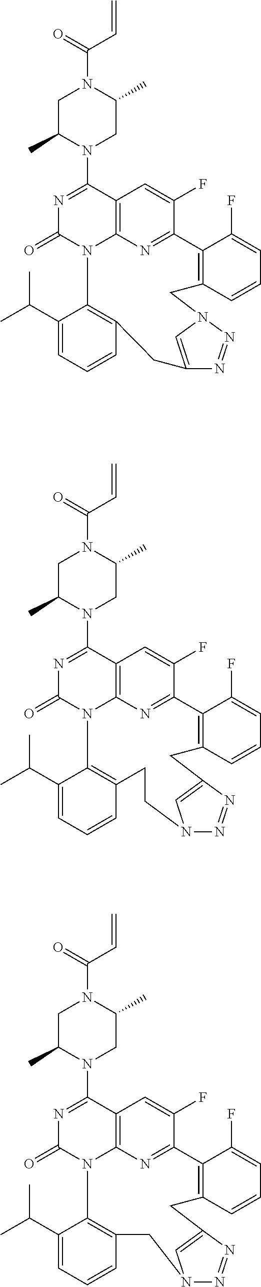 [SMILES: C=CC(=O)N1C[C@H](C)N(c2nc(=O)n3c4nc(c(F)cc24)-c2c(F)cccc2Cn2cc(nn2)Cc2cccc(C(C)C)c2-3)C[C@H]1C ; C=CC(=O)N1C[C@H](C)N(c2nc(=O)n3c4nc(c(F)cc24)-c2c(F)cccc2Cc2cn(nn2)CCc2cccc(C(C)C)c2-3)C[C@H]1C ; C=CC(=O)N1C[C@H](C)N(c2nc(=O)n3c4nc(c(F)cc24)-c2c(F)cccc2Cc2cn(nn2)Cc2cccc(C(C)C)c2-3)C[C@H]1C]

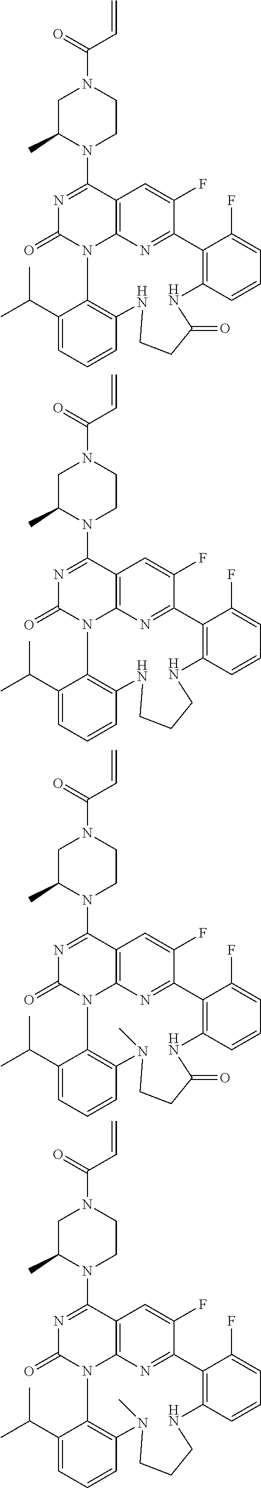

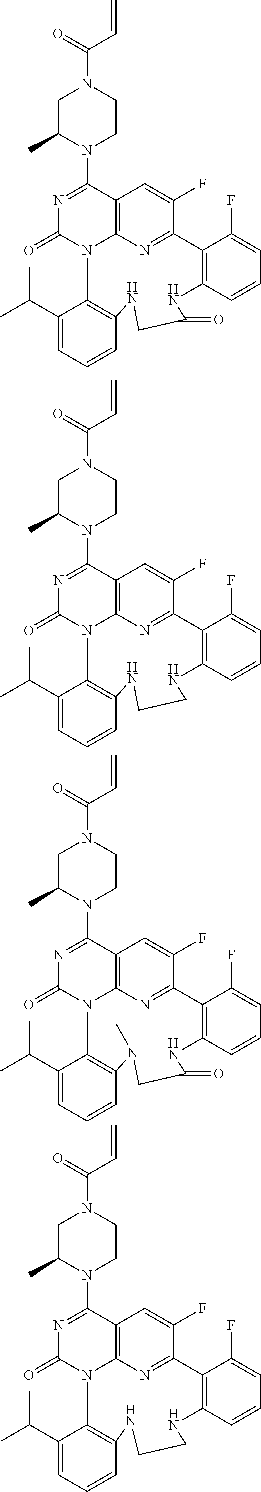

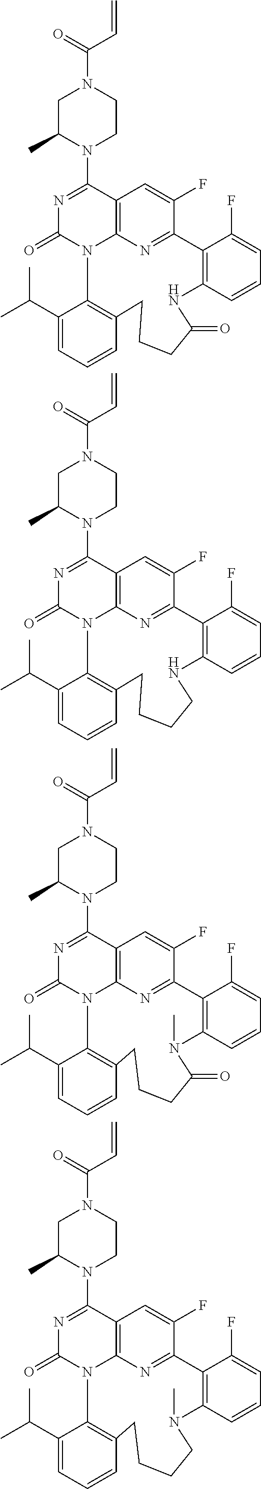

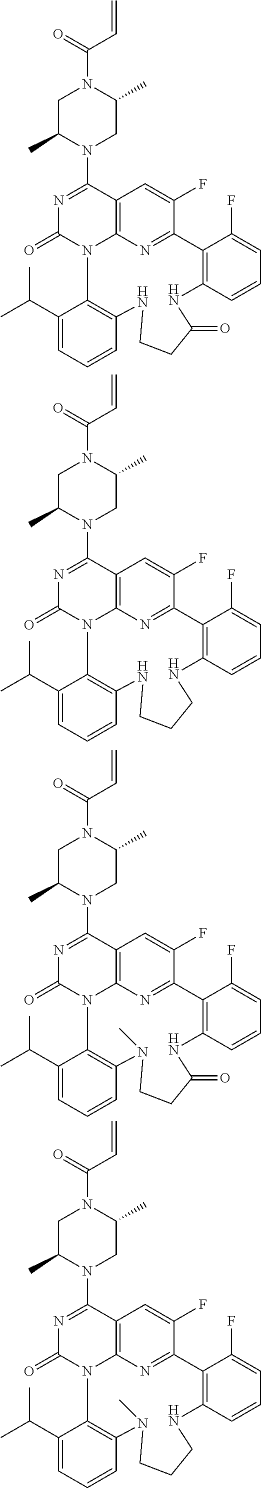

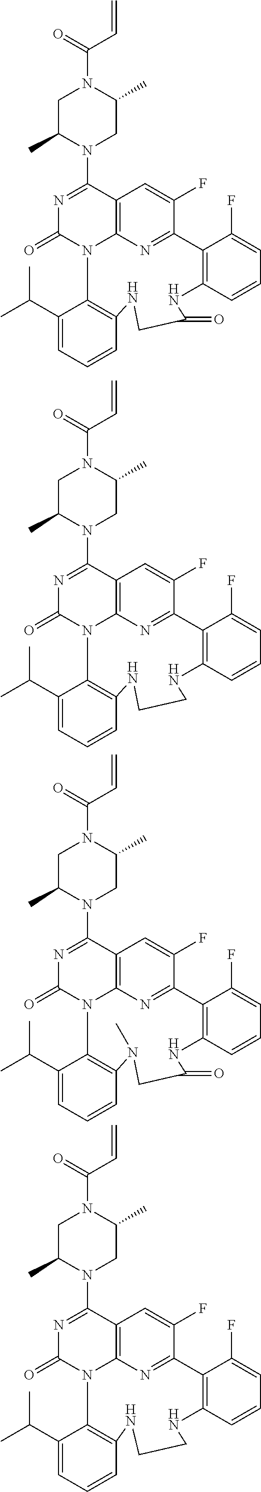

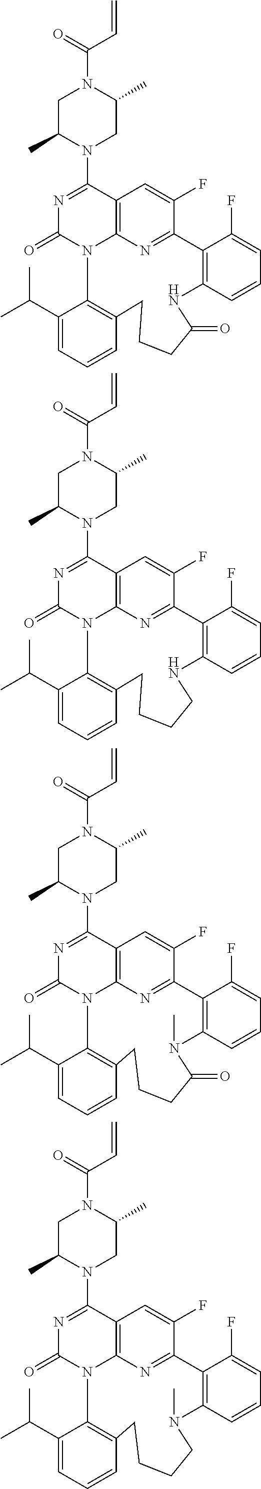

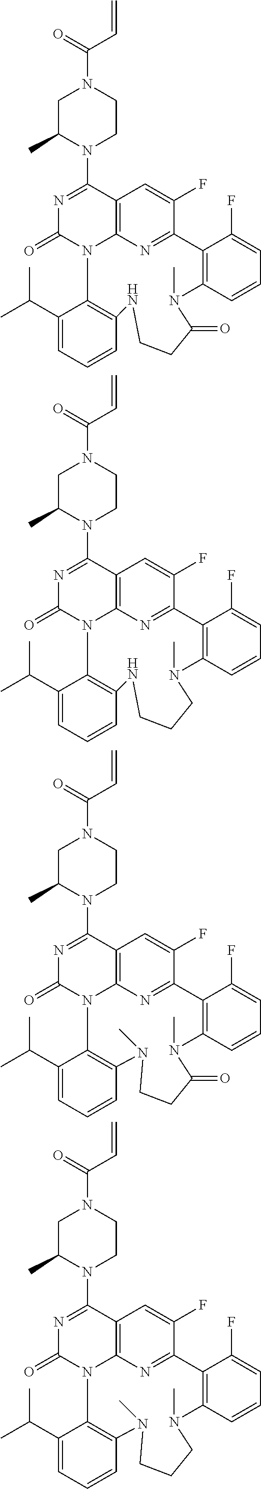

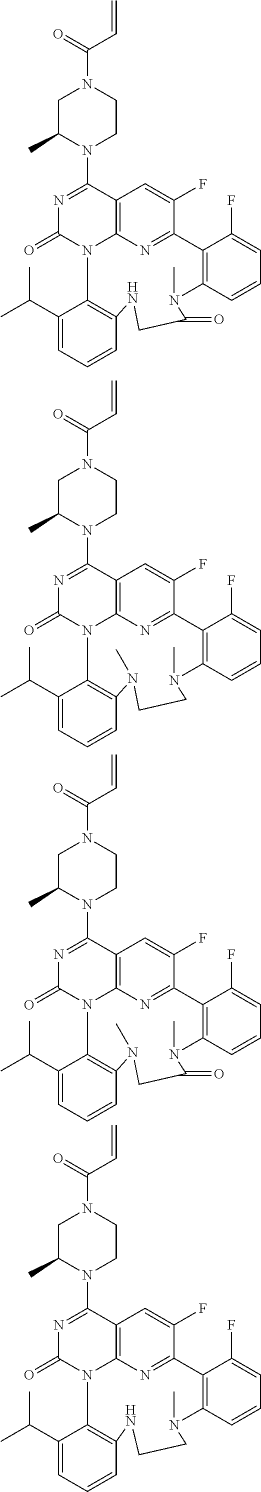

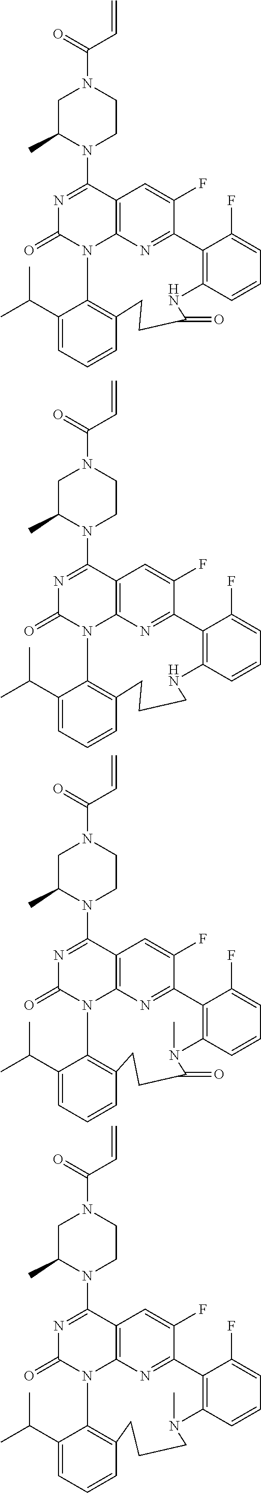

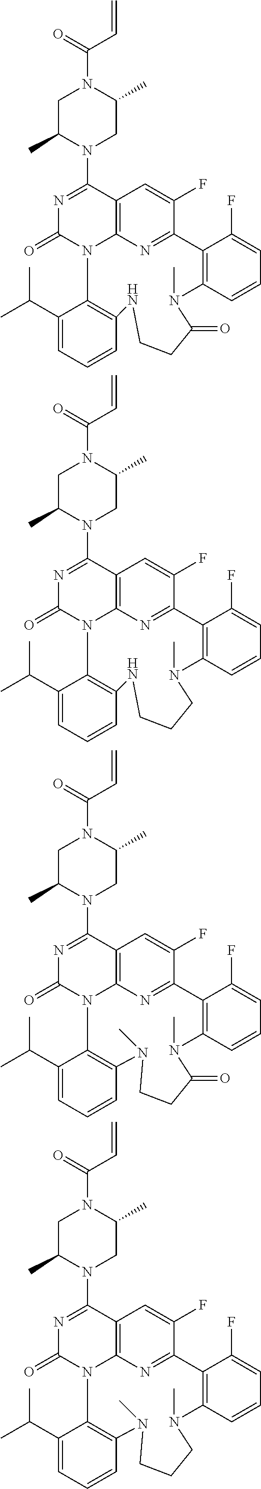

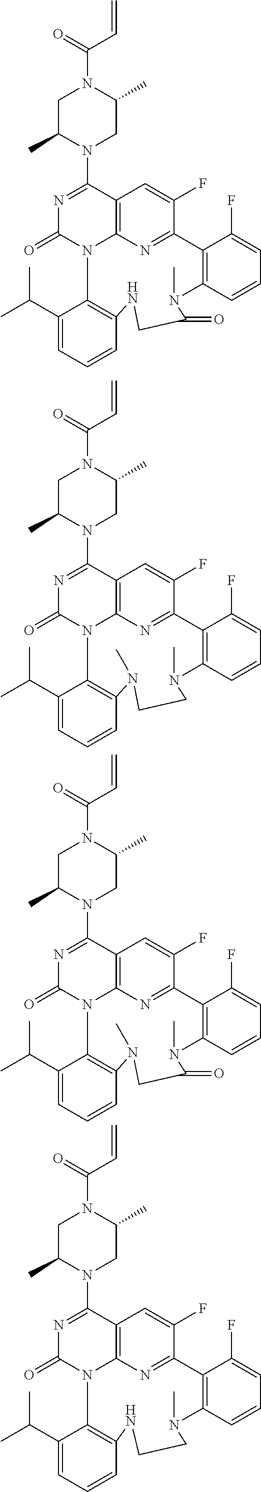

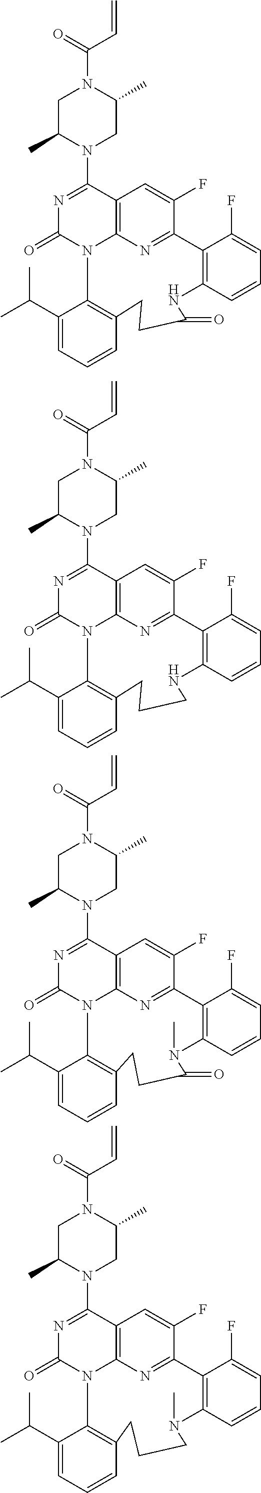

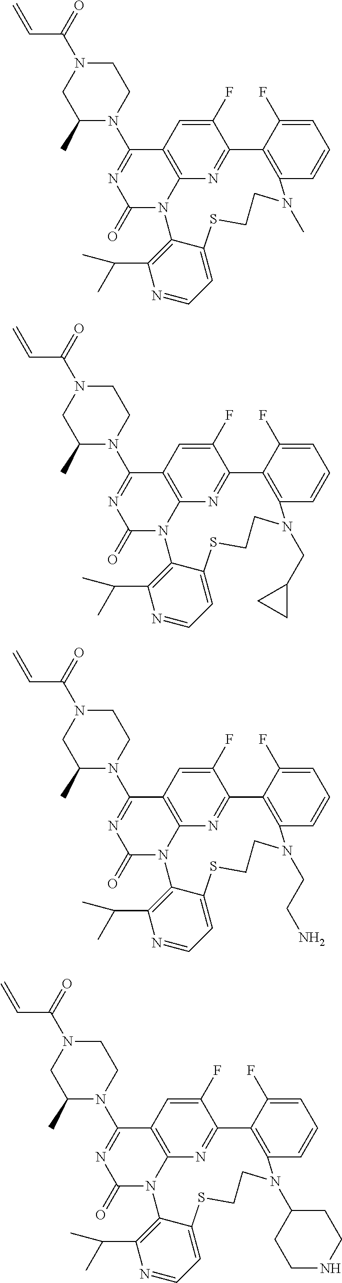

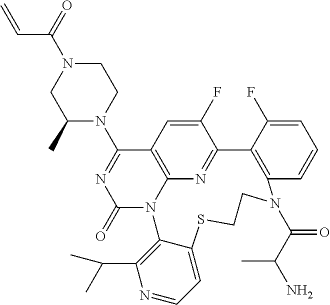 [SMILES: C=CC(=O)N1CCN(c2nc(=O)n3c4nc(c(F)cc24)-c2c(F)cccc2N(C(=O)C(C)N)CCSc2ccnc(C(C)C)c2-3)[C@@H](C)C1]

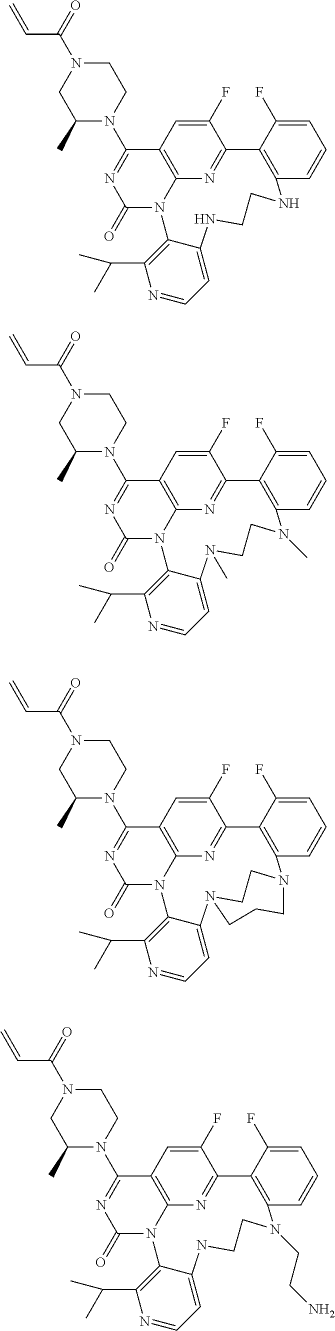 [SMILES: C=CC(=O)N1CCN(c2nc(=O)n3c4nc(c(F)cc24)-c2c(F)cccc2NCCNc2ccnc(C(C)C)c2-3)[C@@H](C)C1 ; C=CC(=O)N1CCN(c2nc(=O)n3c4nc(c(F)cc24)-c2c(F)cccc2N(C)CCN(C)c2ccnc(C(C)C)c2-3)[C@@H](C)C1 ; C=CC(=O)N1CCN(c2nc(=O)n3c4nc(c(F)cc24)-c2c(F)cccc2N2CCCN(CC2)c2ccnc(C(C)C)c2-3)[C@@H](C)C1 ; C=CC(=O)N1CCN(c2nc(=O)n3c4nc(c(F)cc24)-c2c(F)cccc2N(CCN)CCNc2ccnc(C(C)C)c2-3)[C@@H](C)C1]

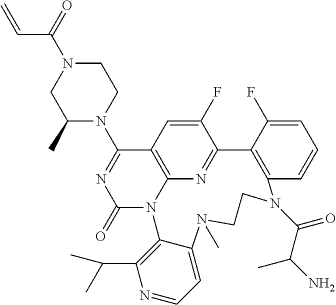 [SMILES: C=CC(=O)N1CCN(c2nc(=O)n3c4nc(c(F)cc24)-c2c(F)cccc2N(C(=O)C(C)N)CCN(C)c2ccnc(C(C)C)c2-3)[C@@H](C)C1]

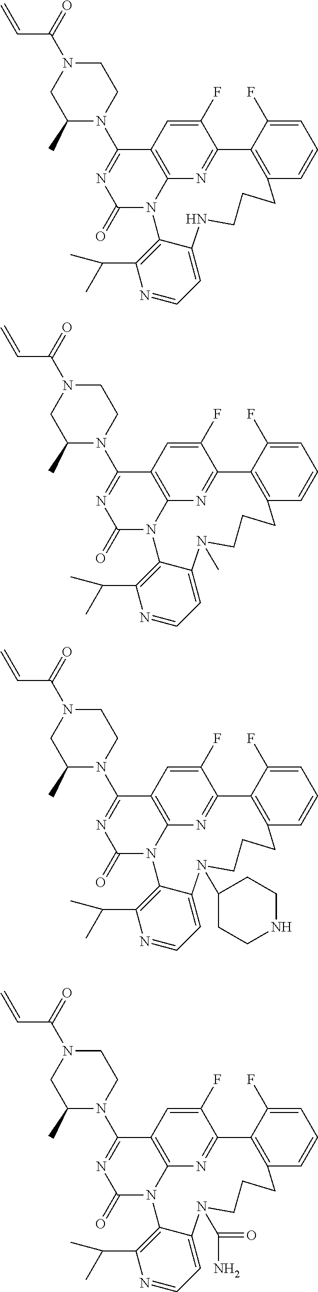

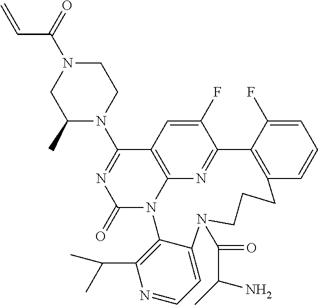 [SMILES: C=CC(=O)N1CCN(c2nc(=O)n3c4nc(c(F)cc24)-c2c(F)cccc2CCCN(C(=O)C(C)N)c2ccnc(C(C)C)c2-3)[C@@H](C)C1]

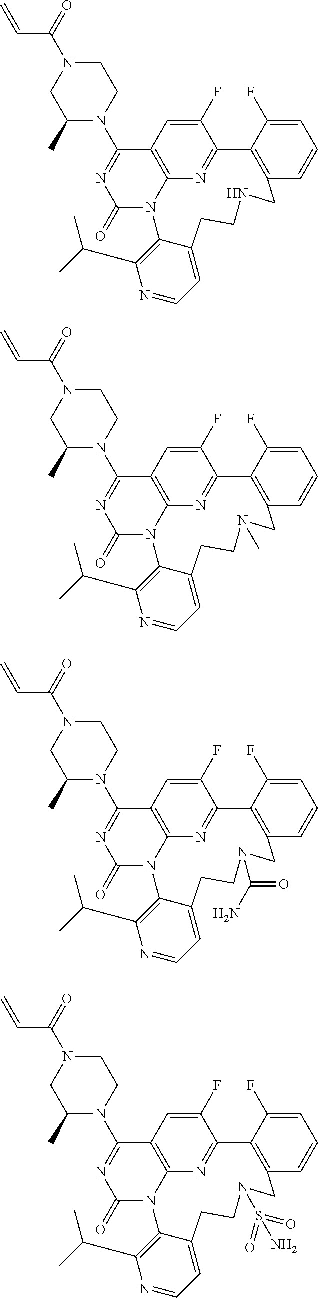 [SMILES: C=CC(=O)N1CCN(c2nc(=O)n3c4nc(c(F)cc24)-c2c(F)cccc2CNCCc2ccnc(C(C)C)c2-3)[C@@H](C)C1 ; C=CC(=O)N1CCN(c2nc(=O)n3c4nc(c(F)cc24)-c2c(F)cccc2CN(C)CCc2ccnc(C(C)C)c2-3)[C@@H](C)C1 ; C=CC(=O)N1CCN(c2nc(=O)n3c4nc(c(F)cc24)-c2c(F)cccc2CN(C(N)=O)CCc2ccnc(C(C)C)c2-3)[C@@H](C)C1 ; C=CC(=O)N1CCN(c2nc(=O)n3c4nc(c(F)cc24)-c2c(F)cccc2CN(S(N)(=O)=O)CCc2ccnc(C(C)C)c2-3)[C@@H](C)C1]

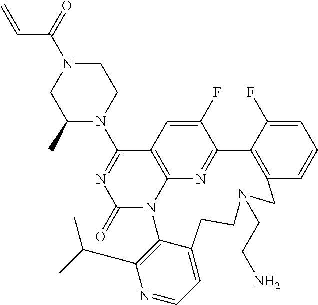 [SMILES: C=CC(=O)N1CCN(c2nc(=O)n3c4nc(c(F)cc24)-c2c(F)cccc2CN(CCN)CCc2ccnc(C(C)C)c2-3)[C@@H](C)C1]

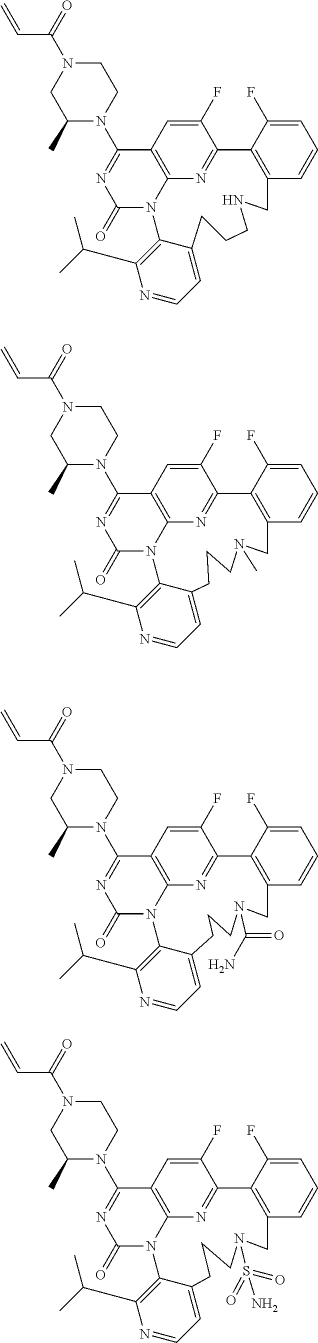 [SMILES: C=CC(=O)N1CCN(c2nc(=O)n3c4nc(c(F)cc24)-c2c(F)cccc2CNCCCc2ccnc(C(C)C)c2-3)[C@@H](C)C1 ; C=CC(=O)N1CCN(c2nc(=O)n3c4nc(c(F)cc24)-c2c(F)cccc2CN(C)CCCc2ccnc(C(C)C)c2-3)[C@@H](C)C1 ; C=CC(=O)N1CCN(c2nc(=O)n3c4nc(c(F)cc24)-c2c(F)cccc2CN(C(N)=O)CCCc2ccnc(C(C)C)c2-3)[C@@H](C)C1 ; C=CC(=O)N1CCN(c2nc(=O)n3c4nc(c(F)cc24)-c2c(F)cccc2CN(S(N)(=O)=O)CCCc2ccnc(C(C)C)c2-3)[C@@H](C)C1]

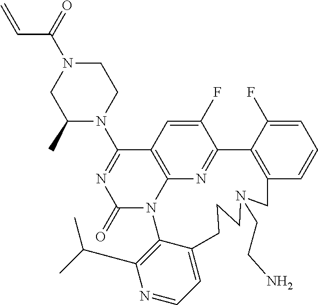 [SMILES: C=CC(=O)N1CCN(c2nc(=O)n3c4nc(c(F)cc24)-c2c(F)cccc2CN(CCN)CCCc2ccnc(C(C)C)c2-3)[C@@H](C)C1]

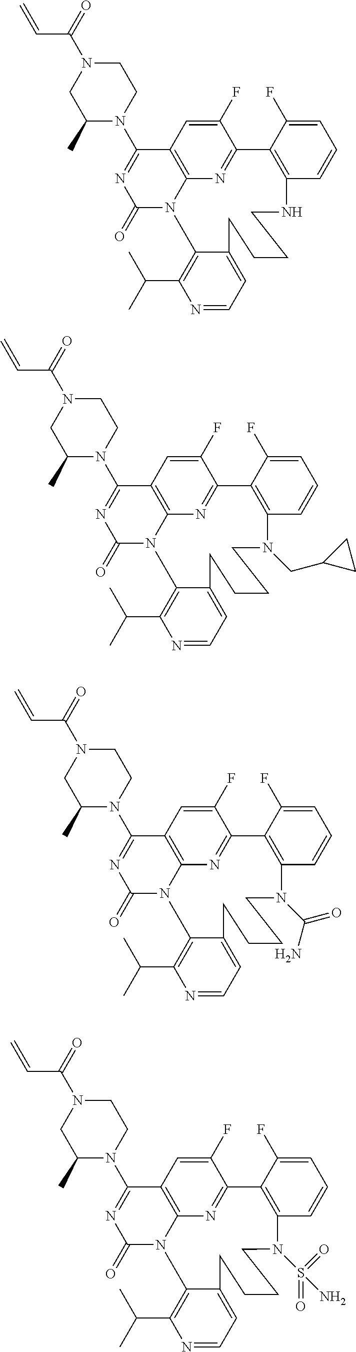 [SMILES: C=CC(=O)N1CCN(c2nc(=O)n3c4nc(c(F)cc24)-c2c(F)cccc2NCCCCc2ccnc(C(C)C)c2-3)[C@@H](C)C1 ; C=CC(=O)N1CCN(c2nc(=O)n3c4nc(c(F)cc24)-c2c(F)cccc2N(CC2CC2)CCCCc2ccnc(C(C)C)c2-3)[C@@H](C)C1 ; C=CC(=O)N1CCN(c2nc(=O)n3c4nc(c(F)cc24)-c2c(F)cccc2N(C(N)=O)CCCCc2ccnc(C(C)C)c2-3)[C@@H](C)C1 ; C=CC(=O)N1CCN(c2nc(=O)n3c4nc(c(F)cc24)-c2c(F)cccc2N(S(N)(=O)=O)CCCCc2ccnc(C(C)C)c2-3)[C@@H](C)C1]

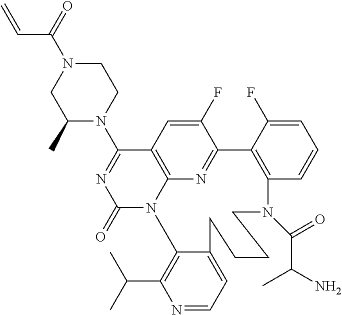 [SMILES: C=CC(=O)N1CCN(c2nc(=O)n3c4nc(c(F)cc24)-c2c(F)cccc2N(C(=O)C(C)N)CCCCc2ccnc(C(C)C)c2-3)[C@@H](C)C1]

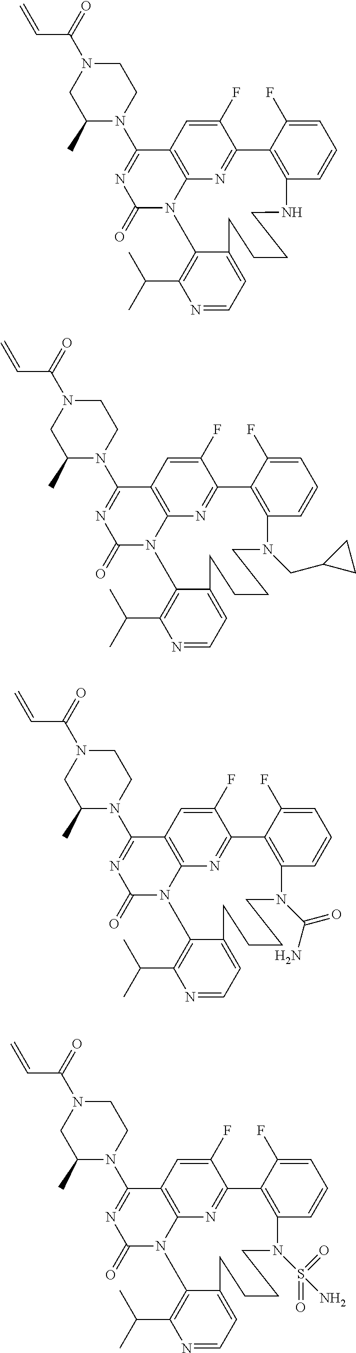 [SMILES: C=CC(=O)N1CCN(c2nc(=O)n3c4nc(c(F)cc24)-c2c(F)cccc2NCCCCc2ccnc(C(C)C)c2-3)[C@@H](C)C1 ; C=CC(=O)N1CCN(c2nc(=O)n3c4nc(c(F)cc24)-c2c(F)cccc2N(CC2CC2)CCCCc2ccnc(C(C)C)c2-3)[C@@H](C)C1 ; C=CC(=O)N1CCN(c2nc(=O)n3c4nc(c(F)cc24)-c2c(F)cccc2N(C(N)=O)CCCCc2ccnc(C(C)C)c2-3)[C@@H](C)C1 ; C=CC(=O)N1CCN(c2nc(=O)n3c4nc(c(F)cc24)-c2c(F)cccc2N(S(N)(=O)=O)CCCCc2ccnc(C(C)C)c2-3)[C@@H](C)C1]

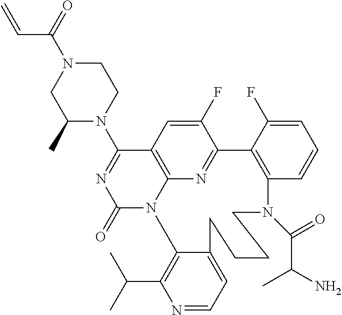 [SMILES: C=CC(=O)N1CCN(c2nc(=O)n3c4nc(c(F)cc24)-c2c(F)cccc2N(C(=O)C(C)N)CCCCc2ccnc(C(C)C)c2-3)[C@@H](C)C1]

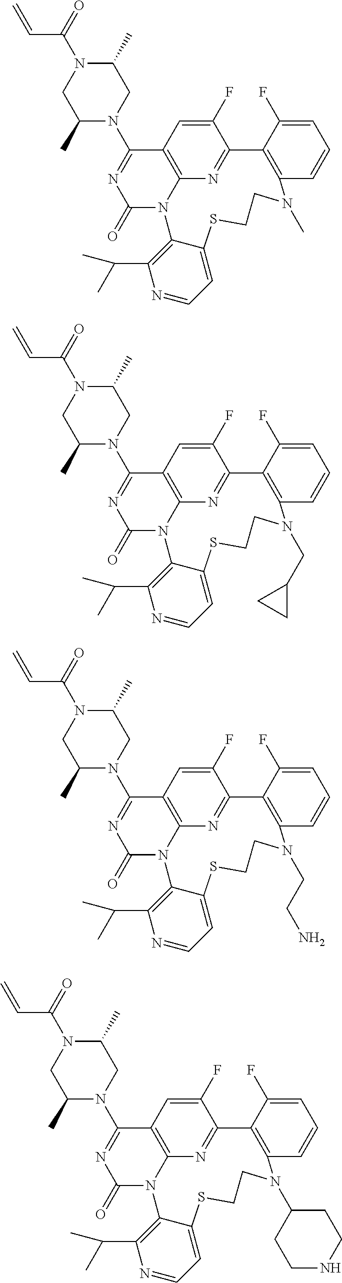

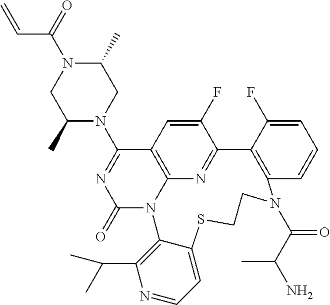 [SMILES: C=CC(=O)N1C[C@H](C)N(c2nc(=O)n3c4nc(c(F)cc24)-c2c(F)cccc2N(C(=O)C(C)N)CCSc2ccnc(C(C)C)c2-3)C[C@H]1C]

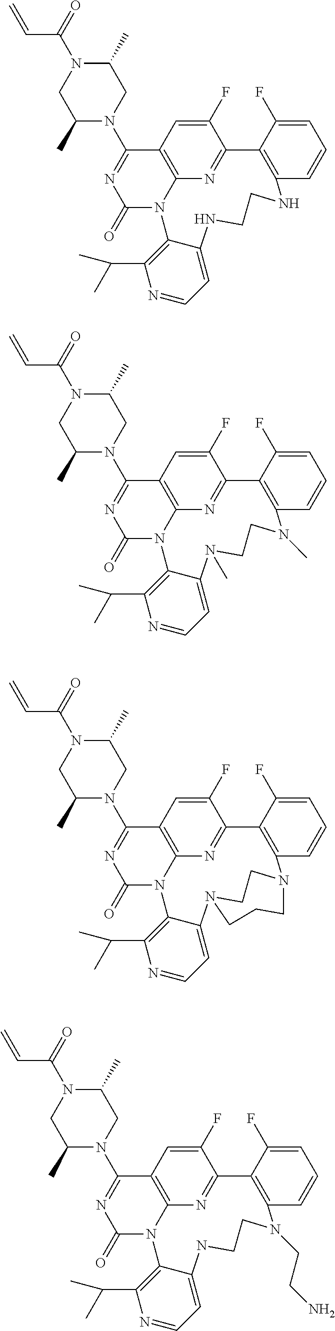 [SMILES: C=CC(=O)N1C[C@H](C)N(c2nc(=O)n3c4nc(c(F)cc24)-c2c(F)cccc2NCCNc2ccnc(C(C)C)c2-3)C[C@H]1C ; C=CC(=O)N1C[C@H](C)N(c2nc(=O)n3c4nc(c(F)cc24)-c2c(F)cccc2N(C)CCN(C)c2ccnc(C(C)C)c2-3)C[C@H]1C ; C=CC(=O)N1C[C@H](C)N(c2nc(=O)n3c4nc(c(F)cc24)-c2c(F)cccc2N2CCCN(CC2)c2ccnc(C(C)C)c2-3)C[C@H]1C ; C=CC(=O)N1C[C@H](C)N(c2nc(=O)n3c4nc(c(F)cc24)-c2c(F)cccc2N(CCN)CCNc2ccnc(C(C)C)c2-3)C[C@H]1C]

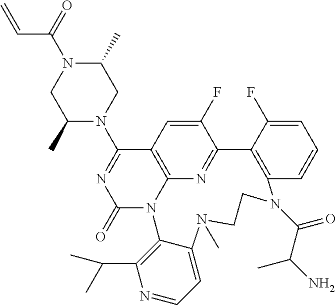 [SMILES: C=CC(=O)N1C[C@H](C)N(c2nc(=O)n3c4nc(c(F)cc24)-c2c(F)cccc2N(C(=O)C(C)N)CCN(C)c2ccnc(C(C)C)c2-3)C[C@H]1C]

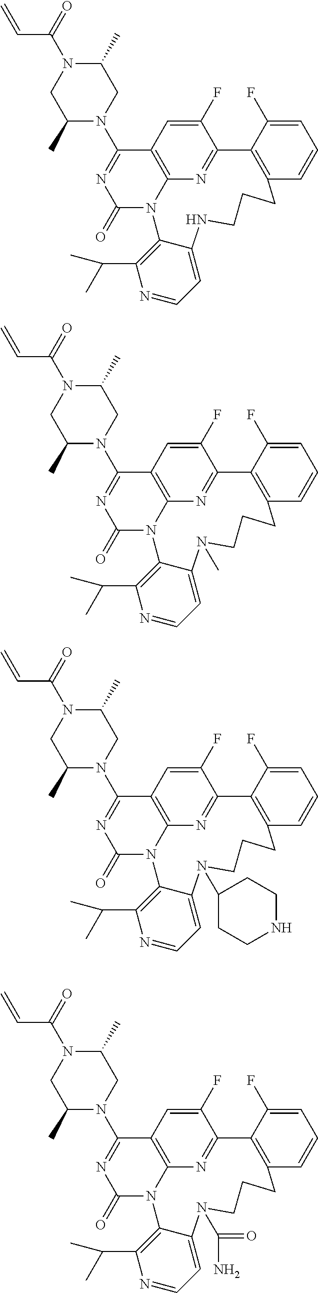

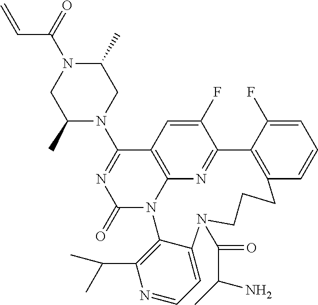 [SMILES: C=CC(=O)N1C[C@H](C)N(c2nc(=O)n3c4nc(c(F)cc24)-c2c(F)cccc2CCCN(C(=O)C(C)N)c2ccnc(C(C)C)c2-3)C[C@H]1C]

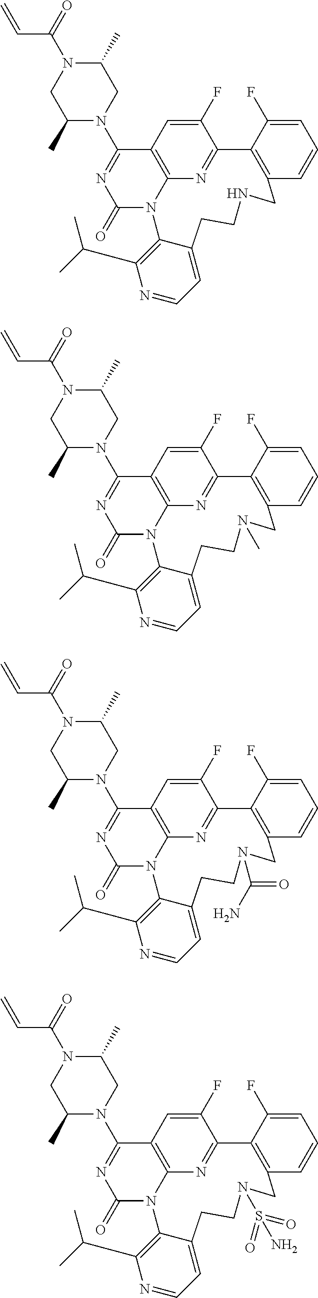 [SMILES: C=CC(=O)N1C[C@H](C)N(c2nc(=O)n3c4nc(c(F)cc24)-c2c(F)cccc2CNCCc2ccnc(C(C)C)c2-3)C[C@H]1C ; C=CC(=O)N1C[C@H](C)N(c2nc(=O)n3c4nc(c(F)cc24)-c2c(F)cccc2CN(C)CCc2ccnc(C(C)C)c2-3)C[C@H]1C ; C=CC(=O)N1C[C@H](C)N(c2nc(=O)n3c4nc(c(F)cc24)-c2c(F)cccc2CN(C(N)=O)CCc2ccnc(C(C)C)c2-3)C[C@H]1C ; C=CC(=O)N1C[C@H](C)N(c2nc(=O)n3c4nc(c(F)cc24)-c2c(F)cccc2CN(S(N)(=O)=O)CCc2ccnc(C(C)C)c2-3)C[C@H]1C]

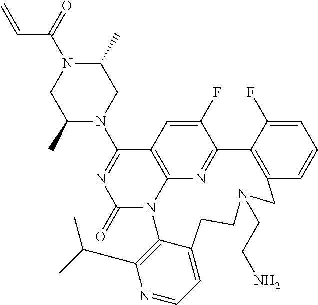 [SMILES: C=CC(=O)N1C[C@H](C)N(c2nc(=O)n3c4nc(c(F)cc24)-c2c(F)cccc2CN(CCN)CCc2ccnc(C(C)C)c2-3)C[C@H]1C]

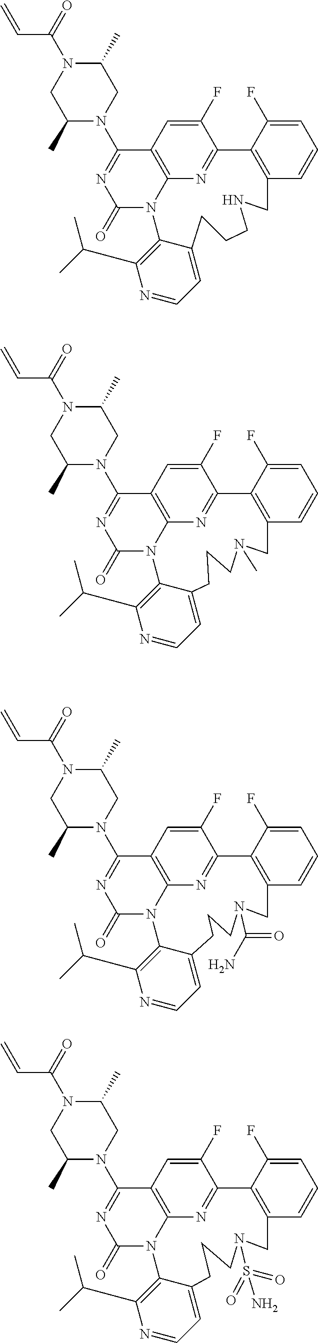 [SMILES: C=CC(=O)N1C[C@H](C)N(c2nc(=O)n3c4nc(c(F)cc24)-c2c(F)cccc2CNCCCc2ccnc(C(C)C)c2-3)C[C@H]1C ; C=CC(=O)N1C[C@H](C)N(c2nc(=O)n3c4nc(c(F)cc24)-c2c(F)cccc2CN(C)CCCc2ccnc(C(C)C)c2-3)C[C@H]1C ; C=CC(=O)N1C[C@H](C)N(c2nc(=O)n3c4nc(c(F)cc24)-c2c(F)cccc2CN(C(N)=O)CCCc2ccnc(C(C)C)c2-3)C[C@H]1C ; C=CC(=O)N1C[C@H](C)N(c2nc(=O)n3c4nc(c(F)cc24)-c2c(F)cccc2CN(S(N)(=O)=O)CCCc2ccnc(C(C)C)c2-3)C[C@H]1C]

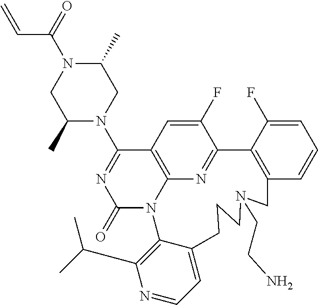 [SMILES: C=CC(=O)N1C[C@H](C)N(c2nc(=O)n3c4nc(c(F)cc24)-c2c(F)cccc2CN(CCN)CCCc2ccnc(C(C)C)c2-3)C[C@H]1C]

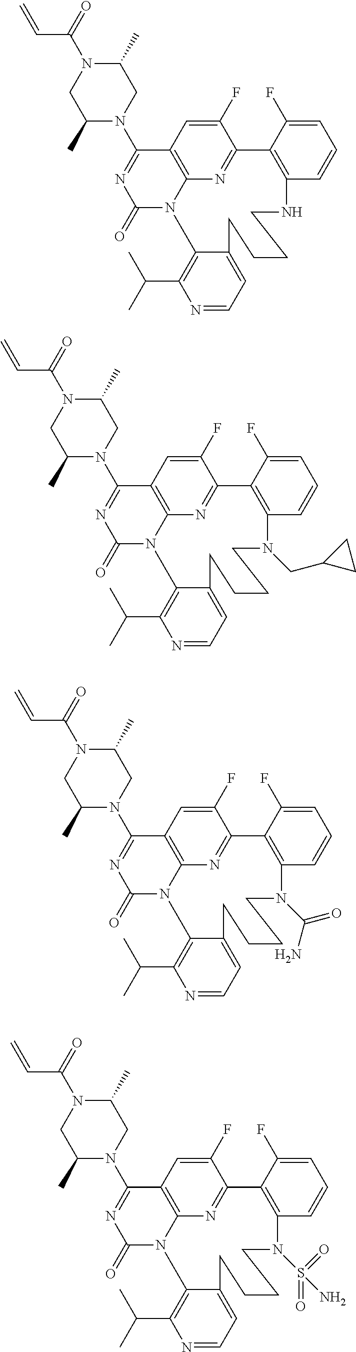 [SMILES: C=CC(=O)N1C[C@H](C)N(c2nc(=O)n3c4nc(c(F)cc24)-c2c(F)cccc2NCCCCc2ccnc(C(C)C)c2-3)C[C@H]1C ; C=CC(=O)N1C[C@H](C)N(c2nc(=O)n3c4nc(c(F)cc24)-c2c(F)cccc2N(CC2CC2)CCCCc2ccnc(C(C)C)c2-3)C[C@H]1C ; C=CC(=O)N1C[C@H](C)N(c2nc(=O)n3c4nc(c(F)cc24)-c2c(F)cccc2N(C(N)=O)CCCCc2ccnc(C(C)C)c2-3)C[C@H]1C ; C=CC(=O)N1C[C@H](C)N(c2nc(=O)n3c4nc(c(F)cc24)-c2c(F)cccc2N(S(N)(=O)=O)CCCCc2ccnc(C(C)C)c2-3)C[C@H]1C]

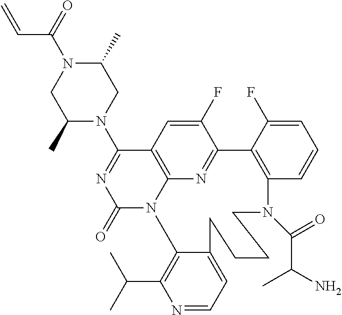 [SMILES: C=CC(=O)N1C[C@H](C)N(c2nc(=O)n3c4nc(c(F)cc24)-c2c(F)cccc2N(C(=O)C(C)N)CCCCc2ccnc(C(C)C)c2-3)C[C@H]1C]

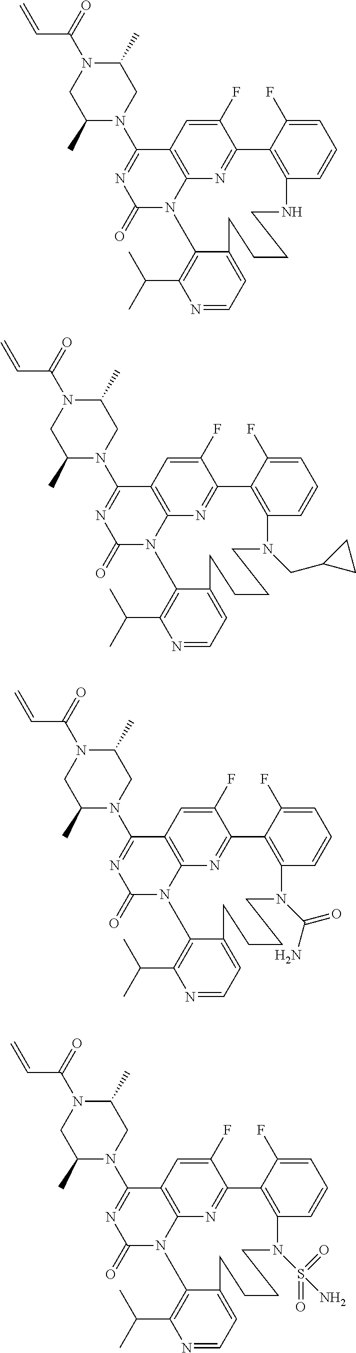 [SMILES: C=CC(=O)N1C[C@H](C)N(c2nc(=O)n3c4nc(c(F)cc24)-c2c(F)cccc2NCCCCc2ccnc(C(C)C)c2-3)C[C@H]1C ; C=CC(=O)N1C[C@H](C)N(c2nc(=O)n3c4nc(c(F)cc24)-c2c(F)cccc2N(CC2CC2)CCCCc2ccnc(C(C)C)c2-3)C[C@H]1C ; C=CC(=O)N1C[C@H](C)N(c2nc(=O)n3c4nc(c(F)cc24)-c2c(F)cccc2N(C(N)=O)CCCCc2ccnc(C(C)C)c2-3)C[C@H]1C ; C=CC(=O)N1C[C@H](C)N(c2nc(=O)n3c4nc(c(F)cc24)-c2c(F)cccc2N(S(N)(=O)=O)CCCCc2ccnc(C(C)C)c2-3)C[C@H]1C]

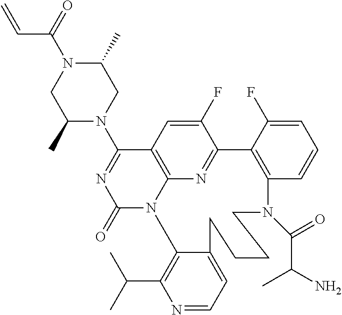 [SMILES: C=CC(=O)N1C[C@H](C)N(c2nc(=O)n3c4nc(c(F)cc24)-c2c(F)cccc2N(C(=O)C(C)N)CCCCc2ccnc(C(C)C)c2-3)C[C@H]1C]

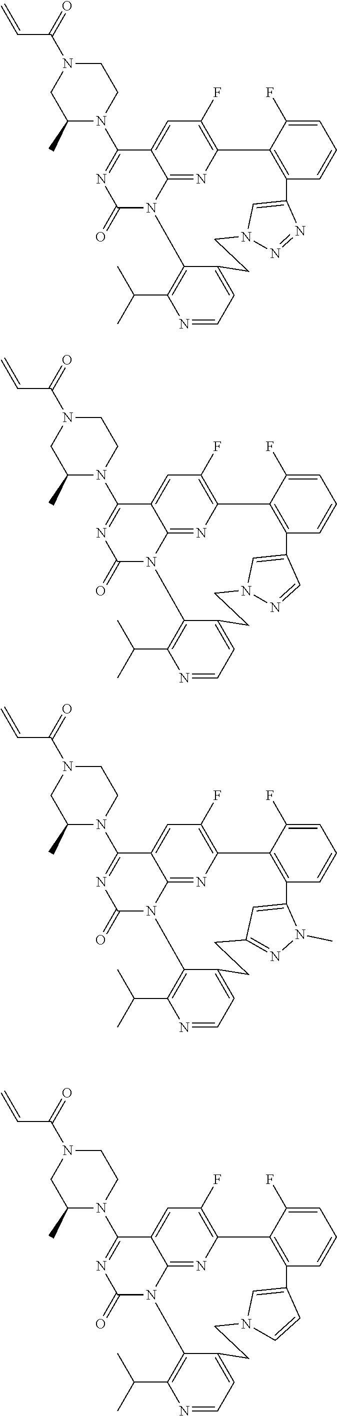 [SMILES: C=CC(=O)N1CCN(c2nc(=O)n3c4nc(c(F)cc24)-c2c(F)cccc2-c2cn(nn2)CCc2ccnc(C(C)C)c2-3)[C@@H](C)C1 ; C=CC(=O)N1CCN(c2nc(=O)n3c4nc(c(F)cc24)-c2c(F)cccc2-c2cnn(c2)CCc2ccnc(C(C)C)c2-3)[C@@H](C)C1 ; C=CC(=O)N1CCN(c2nc(=O)n3c4nc(c(F)cc24)-c2c(F)cccc2-c2cc(nn2C)CCc2ccnc(C(C)C)c2-3)[C@@H](C)C1 ; C=CC(=O)N1CCN(c2nc(=O)n3c4nc(c(F)cc24)-c2c(F)cccc2-c2ccn(c2)CCc2ccnc(C(C)C)c2-3)[C@@H](C)C1]

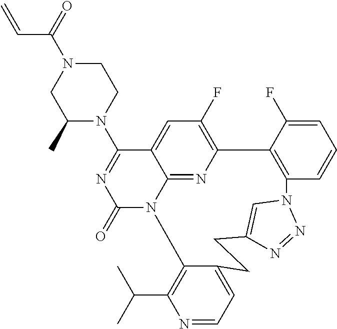 [SMILES: C=CC(=O)N1CCN(c2nc(=O)n3c4nc(c(F)cc24)-c2c(F)cccc2-n2cc(nn2)CCc2ccnc(C(C)C)c2-3)[C@@H](C)C1]

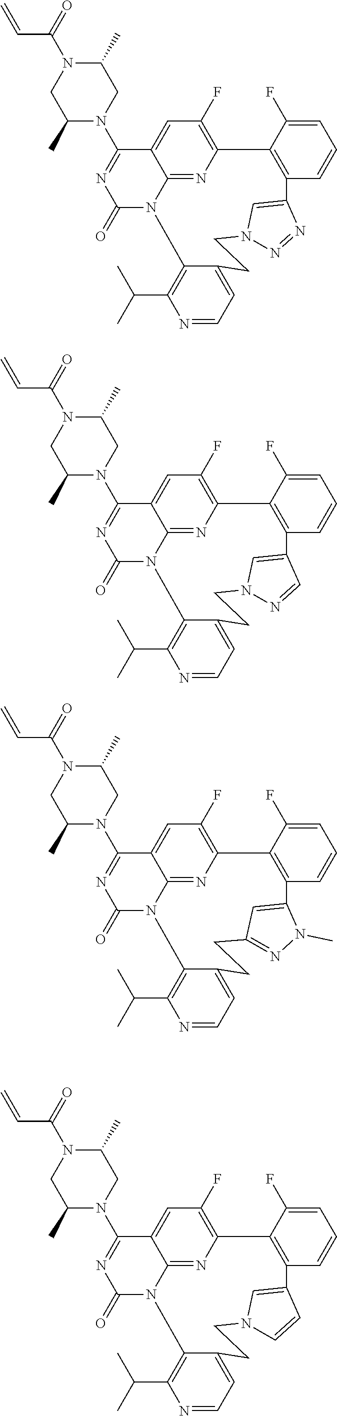 [SMILES: C=CC(=O)N1C[C@H](C)N(c2nc(=O)n3c4nc(c(F)cc24)-c2c(F)cccc2-c2cn(nn2)CCc2ccnc(C(C)C)c2-3)C[C@H]1C ; C=CC(=O)N1C[C@H](C)N(c2nc(=O)n3c4nc(c(F)cc24)-c2c(F)cccc2-c2cnn(c2)CCc2ccnc(C(C)C)c2-3)C[C@H]1C ; C=CC(=O)N1C[C@H](C)N(c2nc(=O)n3c4nc(c(F)cc24)-c2c(F)cccc2-c2cc(nn2C)CCc2ccnc(C(C)C)c2-3)C[C@H]1C ; C=CC(=O)N1C[C@H](C)N(c2nc(=O)n3c4nc(c(F)cc24)-c2c(F)cccc2-c2ccn(c2)CCc2ccnc(C(C)C)c2-3)C[C@H]1C]

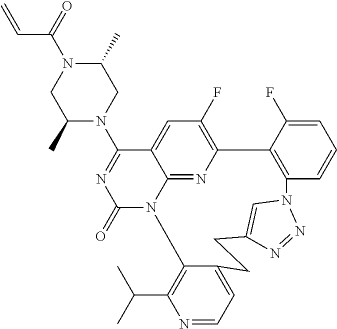 [SMILES: C=CC(=O)N1C[C@H](C)N(c2nc(=O)n3c4nc(c(F)cc24)-c2c(F)cccc2-n2cc(nn2)CCc2ccnc(C(C)C)c2-3)C[C@H]1C]

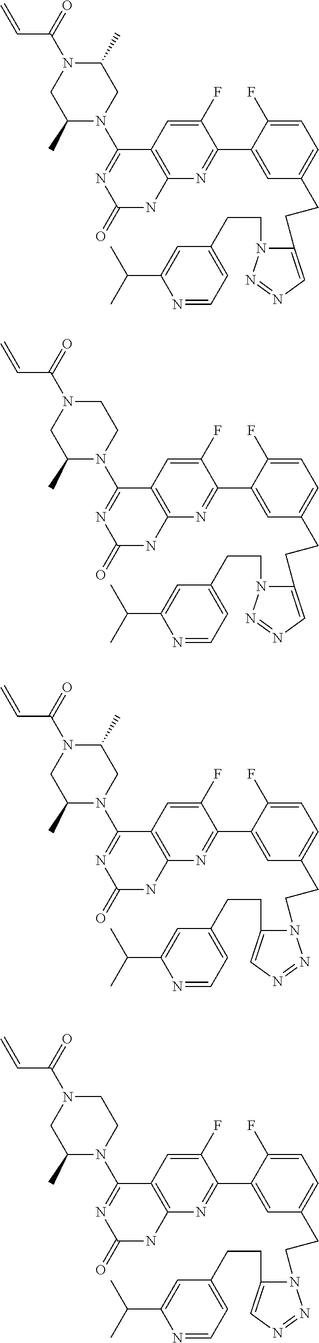 [SMILES: C=CC(=O)N1C[C@H](C)N(c2nc(=O)[nH]c3nc(-c4cc(CCc5cnnn5CCc5ccnc(C(C)C)c5)ccc4F)c(F)cc23)C[C@H]1C ; C=CC(=O)N1CCN(c2nc(=O)[nH]c3nc(-c4cc(CCc5cnnn5CCc5ccnc(C(C)C)c5)ccc4F)c(F)cc23)[C@@H](C)C1 ; C=CC(=O)N1C[C@H](C)N(c2nc(=O)[nH]c3nc(-c4cc(CCn5nncc5CCc5ccnc(C(C)C)c5)ccc4F)c(F)cc23)C[C@H]1C ; C=CC(=O)N1CCN(c2nc(=O)[nH]c3nc(-c4cc(CCn5nncc5CCc5ccnc(C(C)C)c5)ccc4F)c(F)cc23)[C@@H](C)C1]

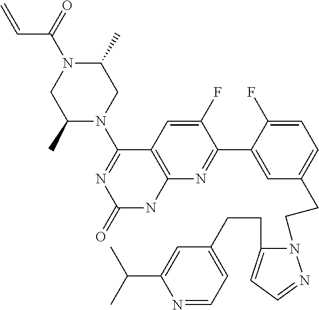 [SMILES: C=CC(=O)N1C[C@H](C)N(c2nc(=O)[nH]c3nc(-c4cc(CCn5nccc5CCc5ccnc(C(C)C)c5)ccc4F)c(F)cc23)C[C@H]1C]

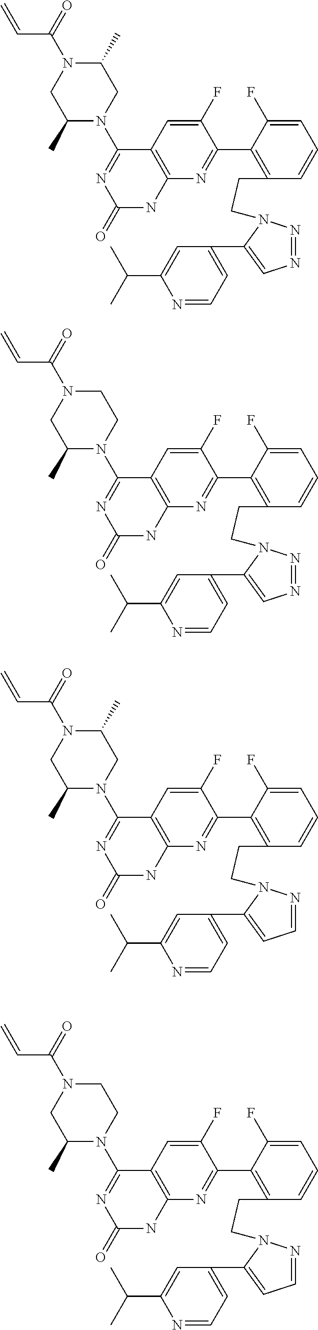 [SMILES: C=CC(=O)N1C[C@H](C)N(c2nc(=O)[nH]c3nc(-c4c(F)cccc4CCn4nncc4-c4ccnc(C(C)C)c4)c(F)cc23)C[C@H]1C ; C=CC(=O)N1CCN(c2nc(=O)[nH]c3nc(-c4c(F)cccc4CCn4nncc4-c4ccnc(C(C)C)c4)c(F)cc23)[C@@H](C)C1 ; C=CC(=O)N1C[C@H](C)N(c2nc(=O)[nH]c3nc(-c4c(F)cccc4CCn4nccc4-c4ccnc(C(C)C)c4)c(F)cc23)C[C@H]1C ; C=CC(=O)N1CCN(c2nc(=O)[nH]c3nc(-c4c(F)cccc4CCn4nccc4-c4ccnc(C(C)C)c4)c(F)cc23)[C@@H](C)C1]

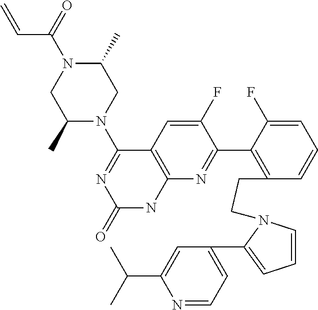 [SMILES: C=CC(=O)N1C[C@H](C)N(c2nc(=O)[nH]c3nc(-c4c(F)cccc4CCn4cccc4-c4ccnc(C(C)C)c4)c(F)cc23)C[C@H]1C]

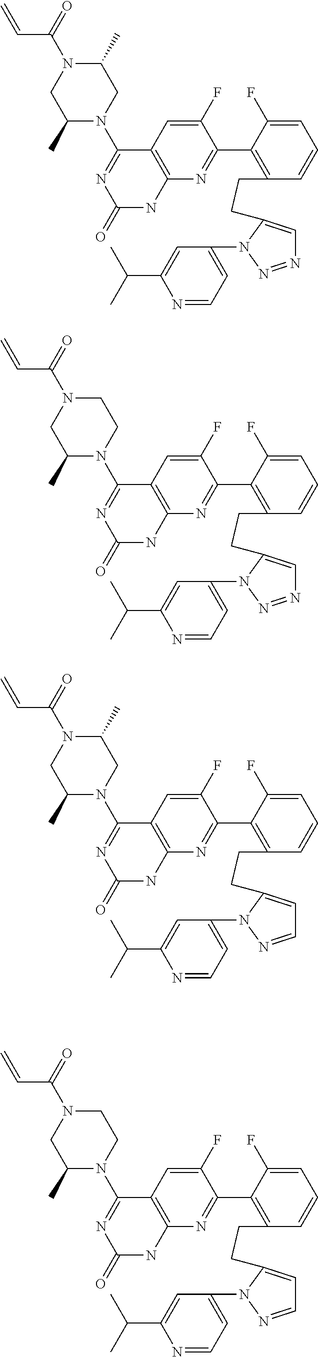 [SMILES: C=CC(=O)N1C[C@H](C)N(c2nc(=O)[nH]c3nc(-c4c(F)cccc4CCc4cnnn4-c4ccnc(C(C)C)c4)c(F)cc23)C[C@H]1C ; C=CC(=O)N1CCN(c2nc(=O)[nH]c3nc(-c4c(F)cccc4CCc4cnnn4-c4ccnc(C(C)C)c4)c(F)cc23)[C@@H](C)C1 ; C=CC(=O)N1C[C@H](C)N(c2nc(=O)[nH]c3nc(-c4c(F)cccc4CCc4ccnn4-c4ccnc(C(C)C)c4)c(F)cc23)C[C@H]1C ; C=CC(=O)N1CCN(c2nc(=O)[nH]c3nc(-c4c(F)cccc4CCc4ccnn4-c4ccnc(C(C)C)c4)c(F)cc23)[C@@H](C)C1]

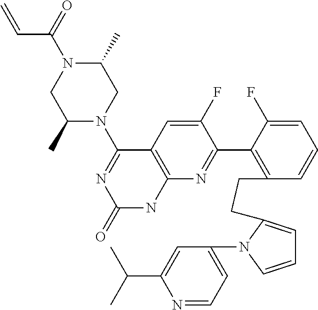 [SMILES: C=CC(=O)N1C[C@H](C)N(c2nc(=O)[nH]c3nc(-c4c(F)cccc4CCc4cccn4-c4ccnc(C(C)C)c4)c(F)cc23)C[C@H]1C]

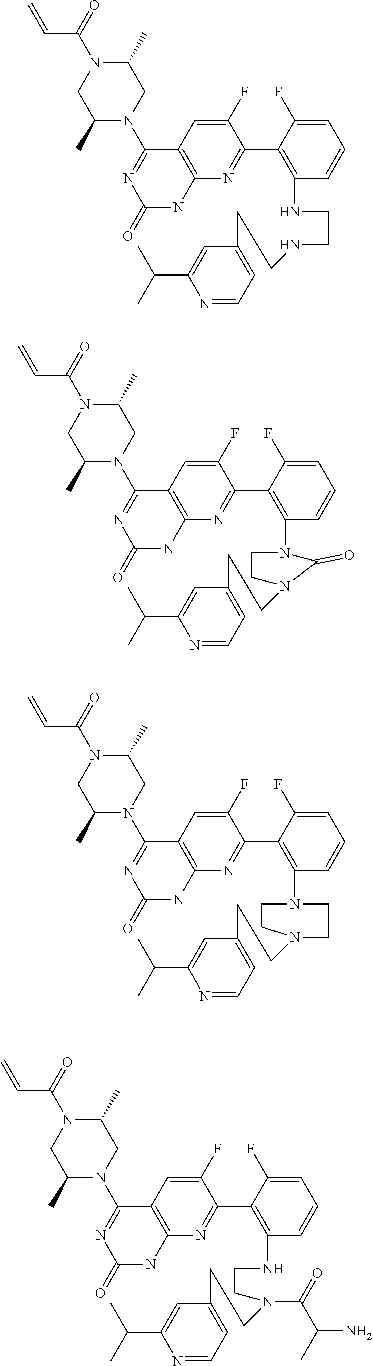 [SMILES: C=CC(=O)N1C[C@H](C)N(c2nc(=O)[nH]c3nc(-c4c(F)cccc4NCCNCCc4ccnc(C(C)C)c4)c(F)cc23)C[C@H]1C ; C=CC(=O)N1C[C@H](C)N(c2nc(=O)[nH]c3nc(-c4c(F)cccc4N4CCN(CCc5ccnc(C(C)C)c5)C4=O)c(F)cc23)C[C@H]1C ; C=CC(=O)N1C[C@H](C)N(c2nc(=O)[nH]c3nc(-c4c(F)cccc4N4CCN(CCc5ccnc(C(C)C)c5)CC4)c(F)cc23)C[C@H]1C ; C=CC(=O)N1C[C@H](C)N(c2nc(=O)[nH]c3nc(-c4c(F)cccc4NCCN(CCc4ccnc(C(C)C)c4)C(=O)C(C)N)c(F)cc23)C[C@H]1C]

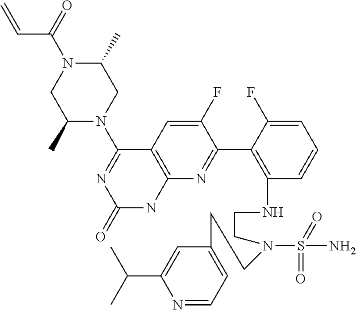 [SMILES: C=CC(=O)N1C[C@H](C)N(c2nc(=O)[nH]c3nc(-c4c(F)cccc4NCCN(CCc4ccnc(C(C)C)c4)S(N)(=O)=O)c(F)cc23)C[C@H]1C]

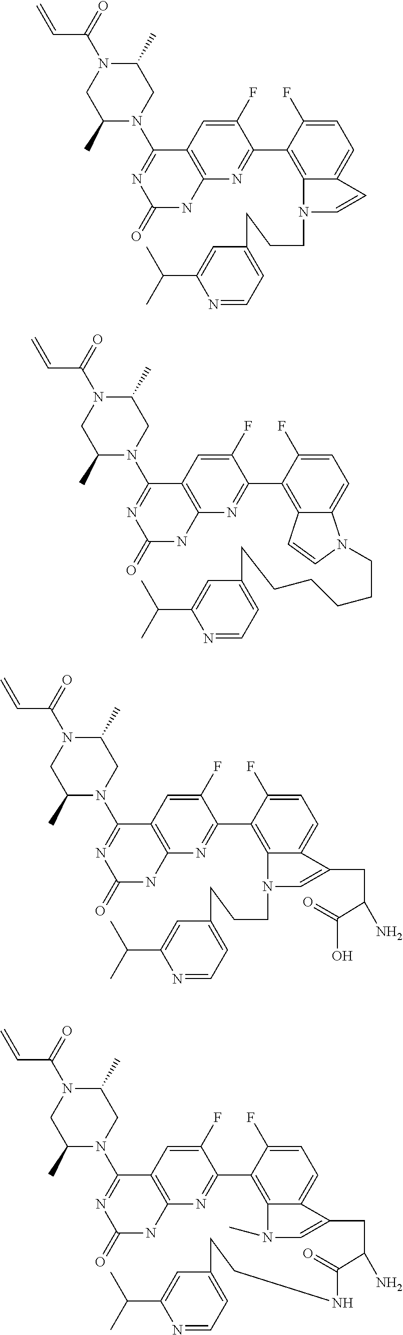 [SMILES: C=CC(=O)N1C[C@H](C)N(c2nc(=O)[nH]c3nc(-c4c(F)ccc5ccn(CCCc6ccnc(C(C)C)c6)c45)c(F)cc23)C[C@H]1C ; C=CC(=O)N1C[C@H](C)N(c2nc(=O)[nH]c3nc(-c4c(F)ccc5c4ccn5CCCCCCc4ccnc(C(C)C)c4)c(F)cc23)C[C@H]1C ; C=CC(=O)N1C[C@H](C)N(c2nc(=O)[nH]c3nc(-c4c(F)ccc5c(CC(N)C(=O)O)cn(CCCc6ccnc(C(C)C)c6)c45)c(F)cc23)C[C@H]1C ; C=CC(=O)N1C[C@H](C)N(c2nc(=O)[nH]c3nc(-c4c(F)ccc5c(CC(N)C(=O)NCCc6ccnc(C(C)C)c6)cn(C)c45)c(F)cc23)C[C@H]1C]

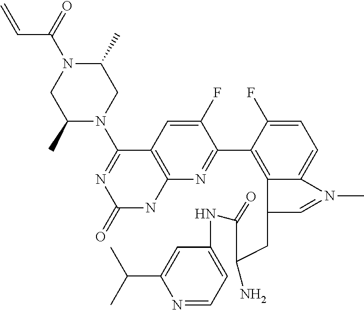

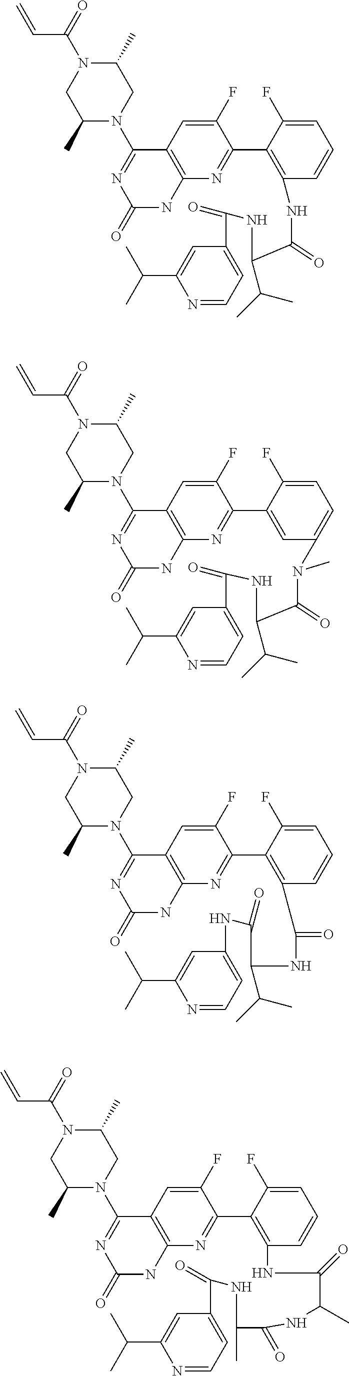 [SMILES: C=CC(=O)N1C[C@H](C)N(c2nc(=O)[nH]c3nc(-c4c(F)cccc4NC(=O)C(NC(=O)c4ccnc(C(C)C)c4)C(C)C)c(F)cc23)C[C@H]1C ; C=CC(=O)N1C[C@H](C)N(c2nc(=O)[nH]c3nc(-c4cc(N(C)C(=O)C(NC(=O)c5ccnc(C(C)C)c5)C(C)C)ccc4F)c(F)cc23)C[C@H]1C ; C=CC(=O)N1C[C@H](C)N(c2nc(=O)[nH]c3nc(-c4c(F)cccc4C(=O)NC(C(=O)Nc4ccnc(C(C)C)c4)C(C)C)c(F)cc23)C[C@H]1C ; C=CC(=O)N1C[C@H](C)N(c2nc(=O)[nH]c3nc(-c4c(F)cccc4NC(=O)C(C)NC(=O)C(C)NC(=O)c4ccnc(C(C)C)c4)c(F)cc23)C[C@H]1C]

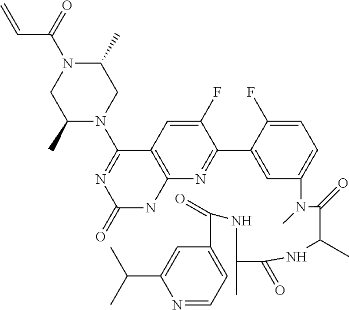 [SMILES: C=CC(=O)N1C[C@H](C)N(c2nc(=O)[nH]c3nc(-c4cc(N(C)C(=O)C(C)NC(=O)C(C)NC(=O)c5ccnc(C(C)C)c5)ccc4F)c(F)cc23)C[C@H]1C]

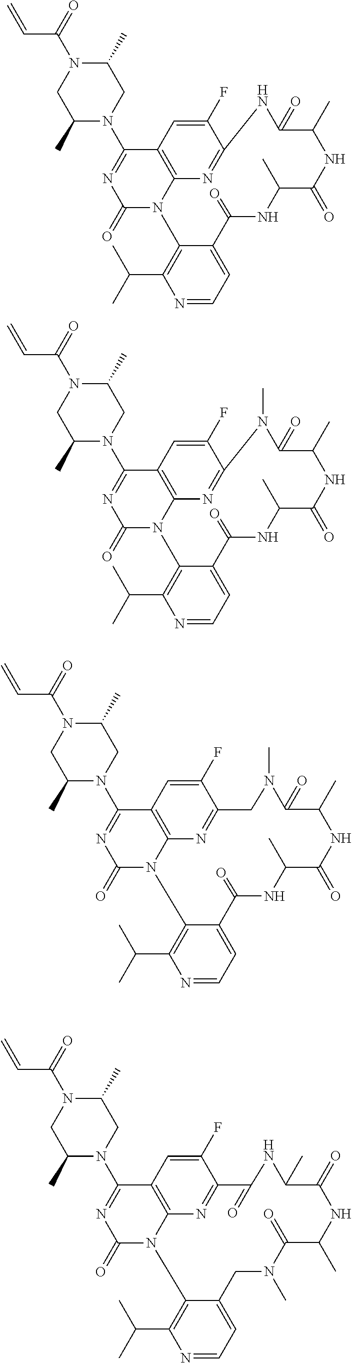

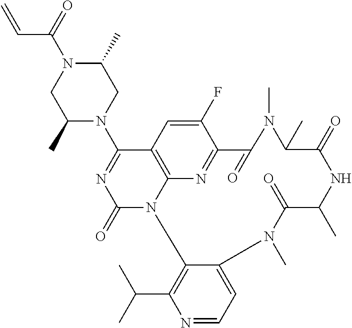 [SMILES: C=CC(=O)N1C[C@H](C)N(c2nc(=O)n3c4nc(c(F)cc24)C(=O)N(C)C(C)C(=O)NC(C)C(=O)N(C)c2ccnc(C(C)C)c2-3)C[C@H]1C]

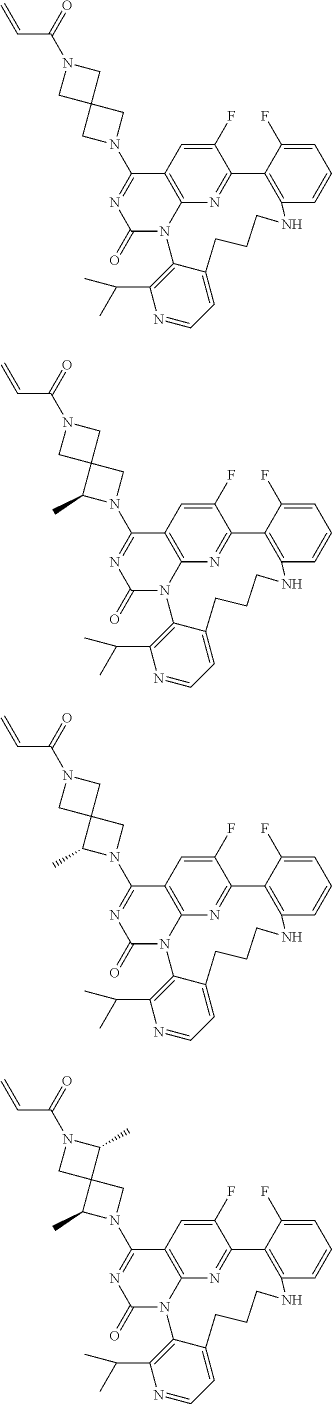 [SMILES: C=CC(=O)N1CC2(C1)CN(c1nc(=O)n3c4nc(c(F)cc14)-c1c(F)cccc1NCCCc1ccnc(C(C)C)c1-3)C2 ; C=CC(=O)N1CC2(C1)CN(c1nc(=O)n3c4nc(c(F)cc14)-c1c(F)cccc1NCCCc1ccnc(C(C)C)c1-3)[C@H]2C ; C=CC(=O)N1CC2(C1)CN(c1nc(=O)n3c4nc(c(F)cc14)-c1c(F)cccc1NCCCc1ccnc(C(C)C)c1-3)[C@@H]2C ; C=CC(=O)N1CC2(CN(c3nc(=O)n4c5nc(c(F)cc35)-c3c(F)cccc3NCCCc3ccnc(C(C)C)c3-4)[C@H]2C)[C@H]1C]

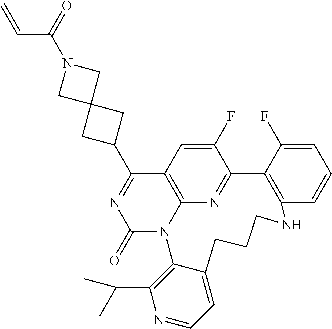 [SMILES: C=CC(=O)N1CC2(CC(c3nc(=O)n4c5nc(c(F)cc35)-c3c(F)cccc3NCCCc3ccnc(C(C)C)c3-4)C2)C1]

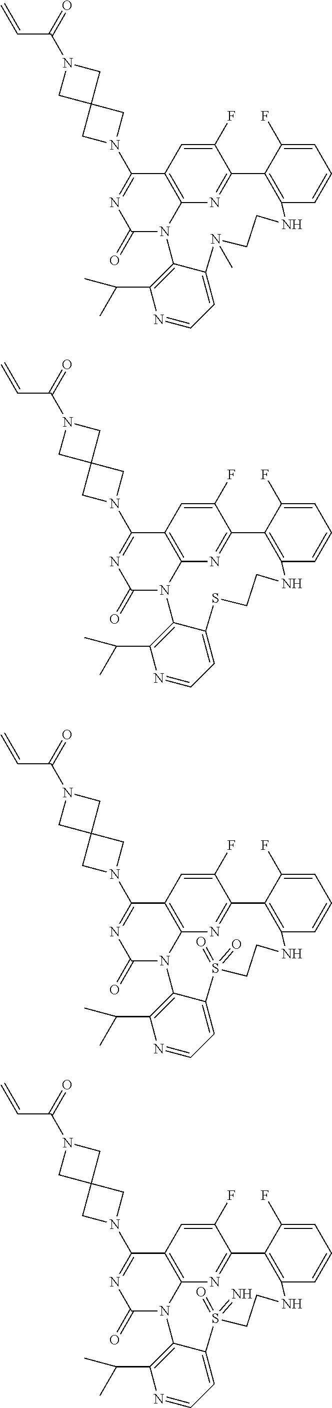 [SMILES: C=CC(=O)N1CC2(C1)CN(c1nc(=O)n3c4nc(c(F)cc14)-c1c(F)cccc1NCCN(C)c1ccnc(C(C)C)c1-3)C2 ; C=CC(=O)N1CC2(C1)CN(c1nc(=O)n3c4nc(c(F)cc14)-c1c(F)cccc1NCCSc1ccnc(C(C)C)c1-3)C2 ; C=CC(=O)N1CC2(C1)CN(c1nc(=O)n3c4nc(c(F)cc14)-c1c(F)cccc1NCCS(=O)(=O)c1ccnc(C(C)C)c1-3)C2 ; C=CC(=O)N1CC2(C1)CN(c1nc(=O)n3c4nc(c(F)cc14)-c1c(F)cccc1NCCS(=N)(=O)c1ccnc(C(C)C)c1-3)C2]

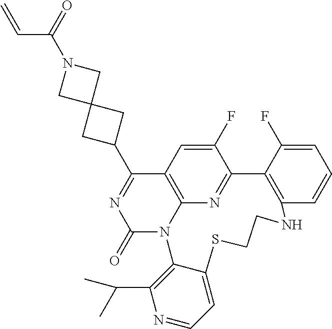 [SMILES: C=CC(=O)N1CC2(CC(c3nc(=O)n4c5nc(c(F)cc35)-c3c(F)cccc3NCCSc3ccnc(C(C)C)c3-4)C2)C1]

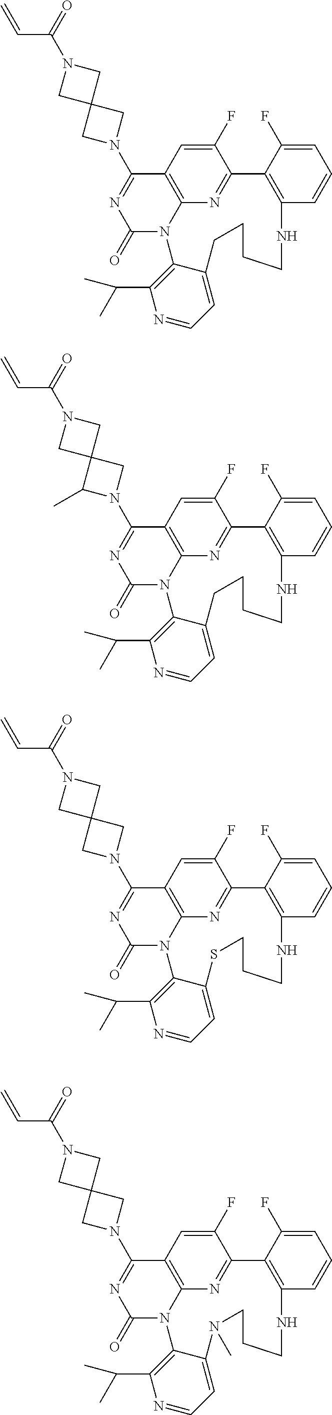 [SMILES: C=CC(=O)N1CC2(C1)CN(c1nc(=O)n3c4nc(c(F)cc14)-c1c(F)cccc1NCCCCc1ccnc(C(C)C)c1-3)C2 ; C=CC(=O)N1CC2(C1)CN(c1nc(=O)n3c4nc(c(F)cc14)-c1c(F)cccc1NCCCCc1ccnc(C(C)C)c1-3)C2C ; C=CC(=O)N1CC2(C1)CN(c1nc(=O)n3c4nc(c(F)cc14)-c1c(F)cccc1NCCCSc1ccnc(C(C)C)c1-3)C2 ; C=CC(=O)N1CC2(C1)CN(c1nc(=O)n3c4nc(c(F)cc14)-c1c(F)cccc1NCCCN(C)c1ccnc(C(C)C)c1-3)C2]

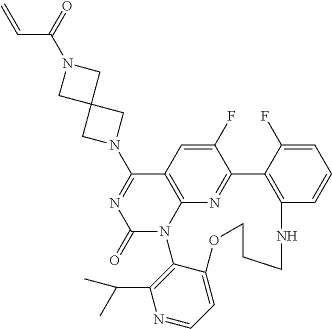 [SMILES: C=CC(=O)N1CC2(C1)CN(c1nc(=O)n3c4nc(c(F)cc14)-c1c(F)cccc1NCCCOc1ccnc(C(C)C)c1-3)C2]

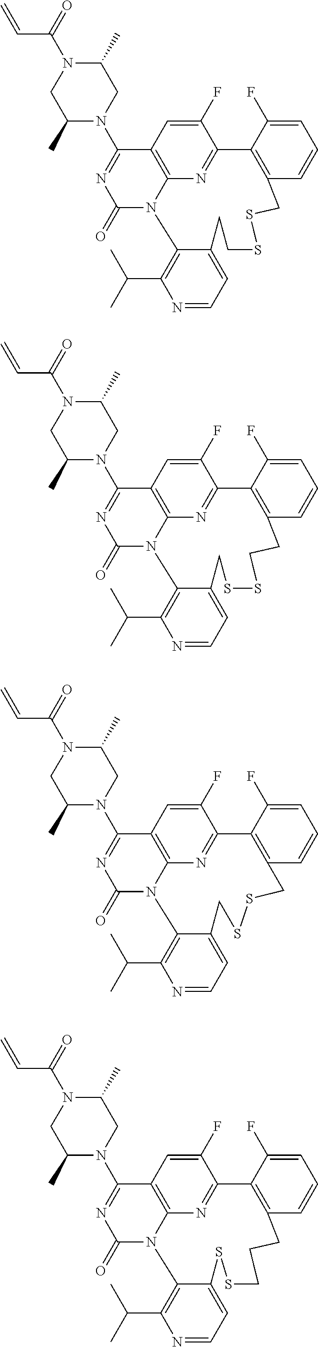 [SMILES: C=CC(=O)N1C[C@H](C)N(c2nc(=O)n3c4nc(c(F)cc24)-c2c(F)cccc2CSSCCc2ccnc(C(C)C)c2-3)C[C@H]1C ; C=CC(=O)N1C[C@H](C)N(c2nc(=O)n3c4nc(c(F)cc24)-c2c(F)cccc2CCSSCc2ccnc(C(C)C)c2-3)C[C@H]1C ; C=CC(=O)N1C[C@H](C)N(c2nc(=O)n3c4nc(c(F)cc24)-c2c(F)cccc2CSSCc2ccnc(C(C)C)c2-3)C[C@H]1C ; C=CC(=O)N1C[C@H](C)N(c2nc(=O)n3c4nc(c(F)cc24)-c2c(F)cccc2CCCSSc2ccnc(C(C)C)c2-3)C[C@H]1C]

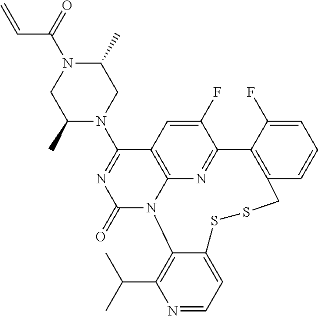 [SMILES: C=CC(=O)N1C[C@H](C)N(c2nc(=O)n3c4nc(c(F)cc24)-c2c(F)cccc2CSSc2ccnc(C(C)C)c2-3)C[C@H]1C]

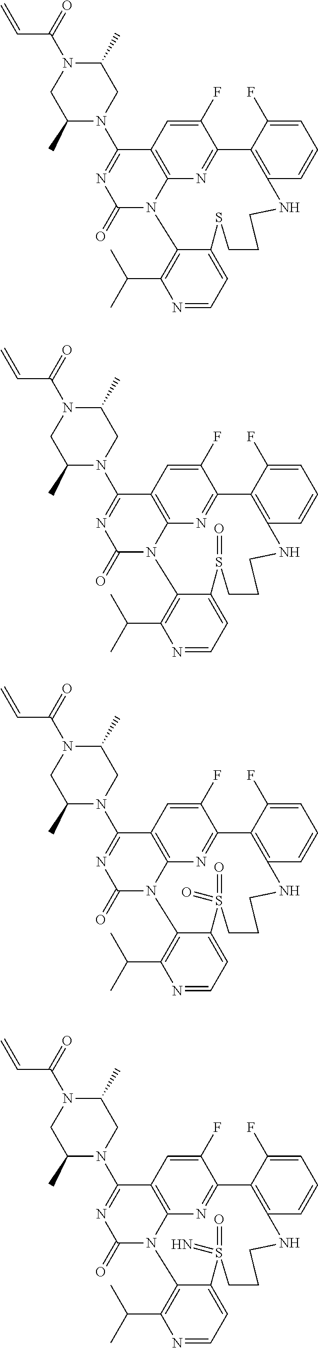 [SMILES: C=CC(=O)N1C[C@H](C)N(c2nc(=O)n3c4nc(c(F)cc24)-c2c(F)cccc2NCCCSc2ccnc(C(C)C)c2-3)C[C@H]1C ; C=CC(=O)N1C[C@H](C)N(c2nc(=O)n3c4nc(c(F)cc24)-c2c(F)cccc2NCCCS(=O)c2ccnc(C(C)C)c2-3)C[C@H]1C ; C=CC(=O)N1C[C@H](C)N(c2nc(=O)n3c4nc(c(F)cc24)-c2c(F)cccc2NCCCS(=O)(=O)c2ccnc(C(C)C)c2-3)C[C@H]1C ; C=CC(=O)N1C[C@H](C)N(c2nc(=O)n3c4nc(c(F)cc24)-c2c(F)cccc2NCCCS(=N)(=O)c2ccnc(C(C)C)c2-3)C[C@H]1C]

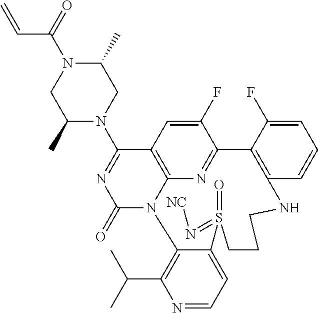 [SMILES: C=CC(=O)N1C[C@H](C)N(c2nc(=O)n3c4nc(c(F)cc24)-c2c(F)cccc2NCCCS(=O)(=NC#N)c2ccnc(C(C)C)c2-3)C[C@H]1C]

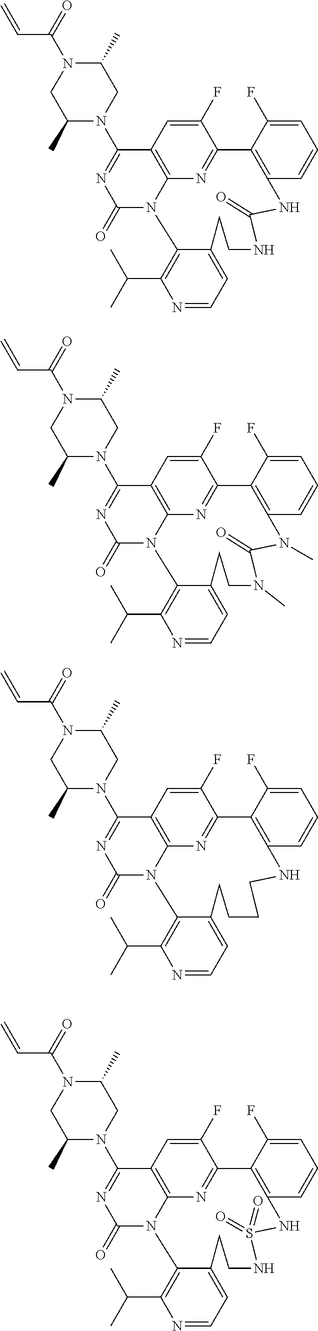 [SMILES: C=CC(=O)N1C[C@H](C)N(c2nc(=O)n3c4nc(c(F)cc24)-c2c(F)cccc2NC(=O)NCCc2ccnc(C(C)C)c2-3)C[C@H]1C ; C=CC(=O)N1C[C@H](C)N(c2nc(=O)n3c4nc(c(F)cc24)-c2c(F)cccc2N(C)C(=O)N(C)CCc2ccnc(C(C)C)c2-3)C[C@H]1C ; C=CC(=O)N1C[C@H](C)N(c2nc(=O)n3c4nc(c(F)cc24)-c2c(F)cccc2NCCCCc2ccnc(C(C)C)c2-3)C[C@H]1C ; C=CC(=O)N1C[C@H](C)N(c2nc(=O)n3c4nc(c(F)cc24)-c2c(F)cccc2NS(=O)(=O)NCCc2ccnc(C(C)C)c2-3)C[C@H]1C]

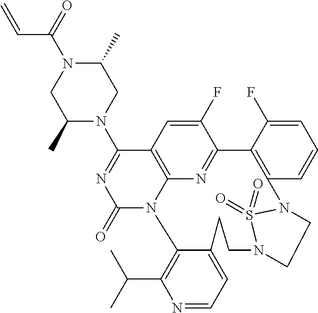 [SMILES: C=CC(=O)N1C[C@H](C)N(c2nc(=O)n3c4nc(c(F)cc24)-c2c(F)cccc2N2CCN(CCc4ccnc(C(C)C)c4-3)S2(=O)=O)C[C@H]1C]

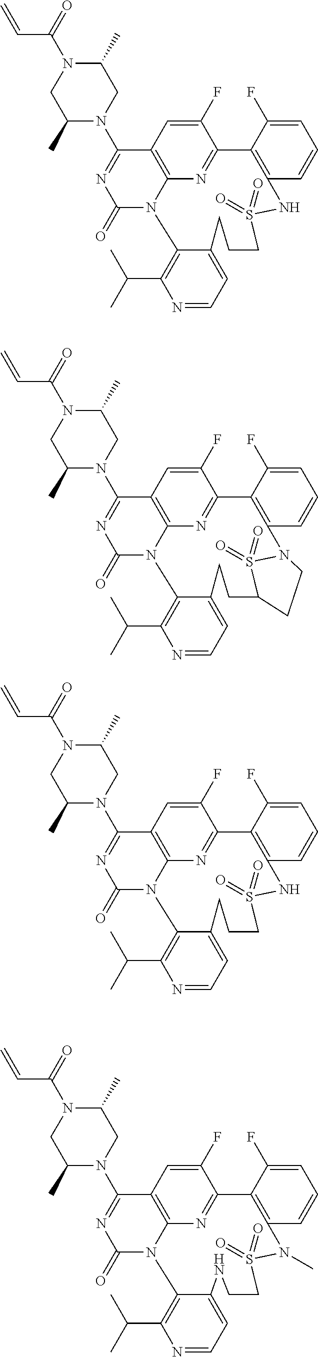 [SMILES: C=CC(=O)N1C[C@H](C)N(c2nc(=O)n3c4nc(c(F)cc24)-c2c(F)cccc2NS(=O)(=O)CCCc2ccnc(C(C)C)c2-3)C[C@H]1C ; C=CC(=O)N1C[C@H](C)N(c2nc(=O)n3c4nc(c(F)cc24)-c2c(F)cccc2N2CCC(CCc4ccnc(C(C)C)c4-3)S2(=O)=O)C[C@H]1C ; C=CC(=O)N1C[C@H](C)N(c2nc(=O)n3c4nc(c(F)cc24)-c2c(F)cccc2NS(=O)(=O)CCCc2ccnc(C(C)C)c2-3)C[C@H]1C ; C=CC(=O)N1C[C@H](C)N(c2nc(=O)n3c4nc(c(F)cc24)-c2c(F)cccc2N(C)S(=O)(=O)CCNc2ccnc(C(C)C)c2-3)C[C@H]1C]

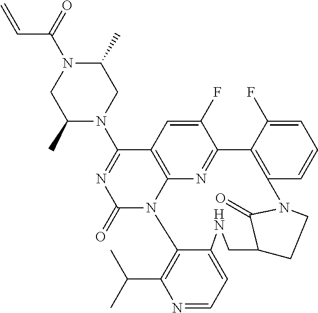 [SMILES: C=CC(=O)N1C[C@H](C)N(c2nc(=O)n3c4nc(c(F)cc24)-c2c(F)cccc2N2CCC(CNc4ccnc(C(C)C)c4-3)C2=O)C[C@H]1C]

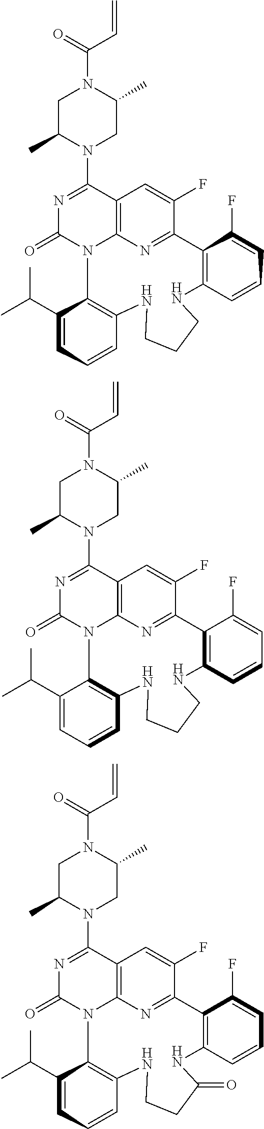

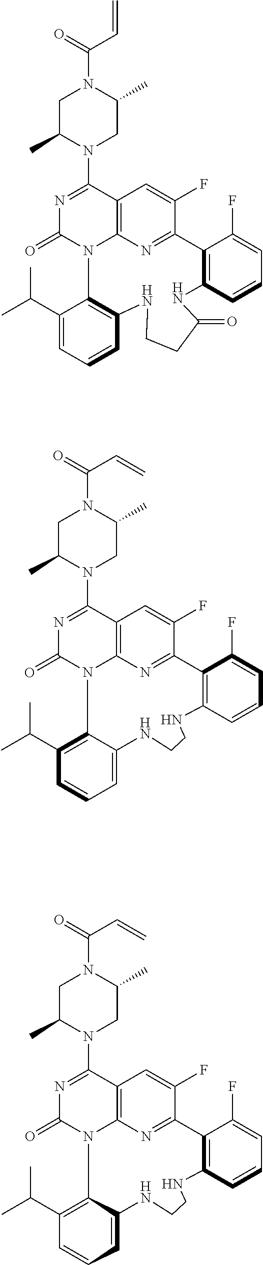

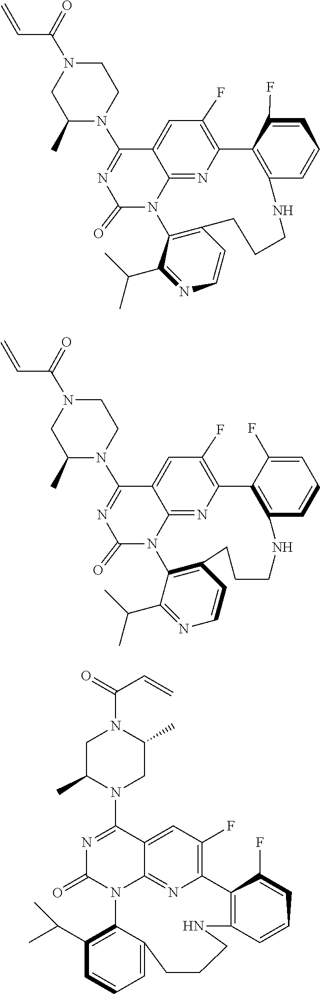

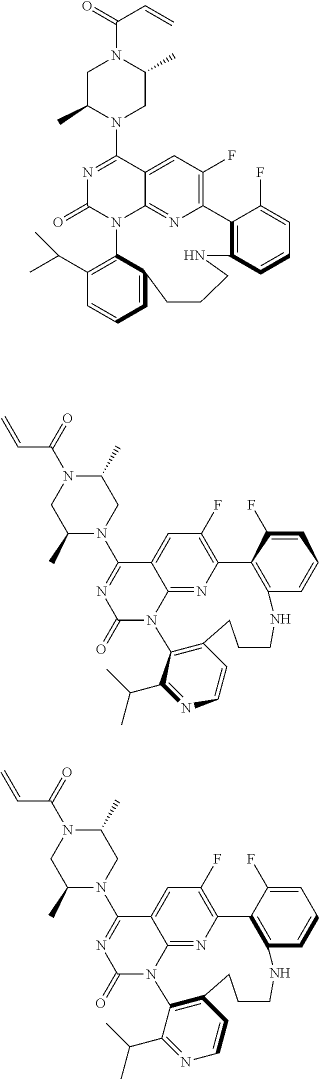

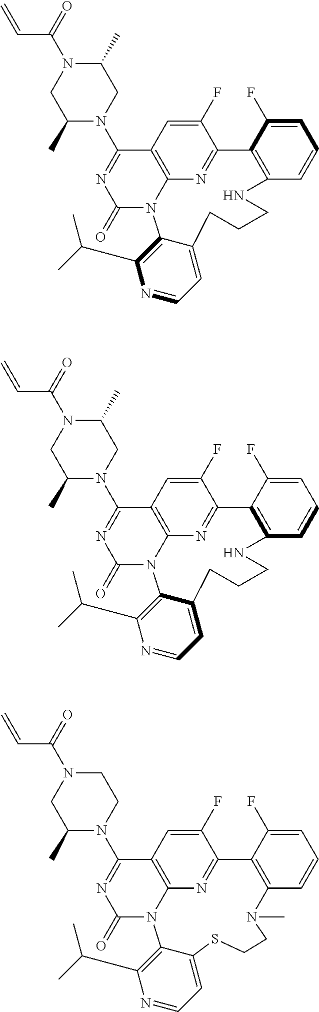

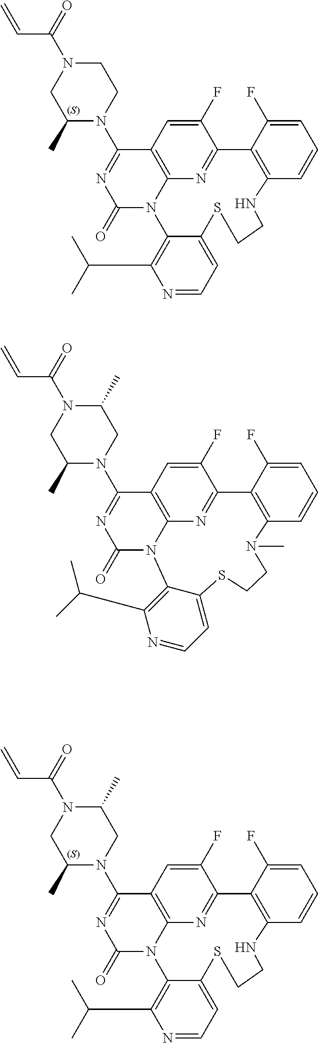

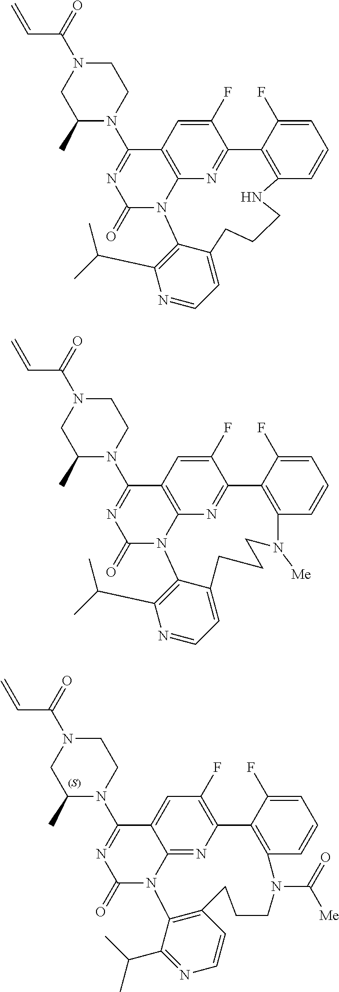

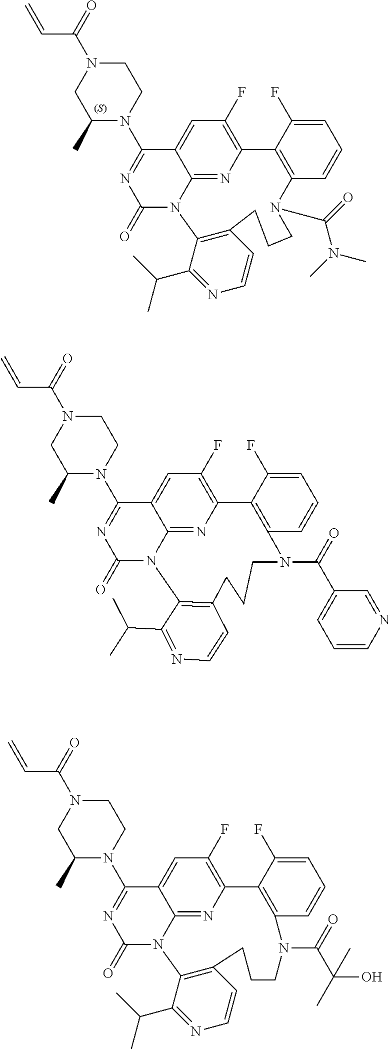

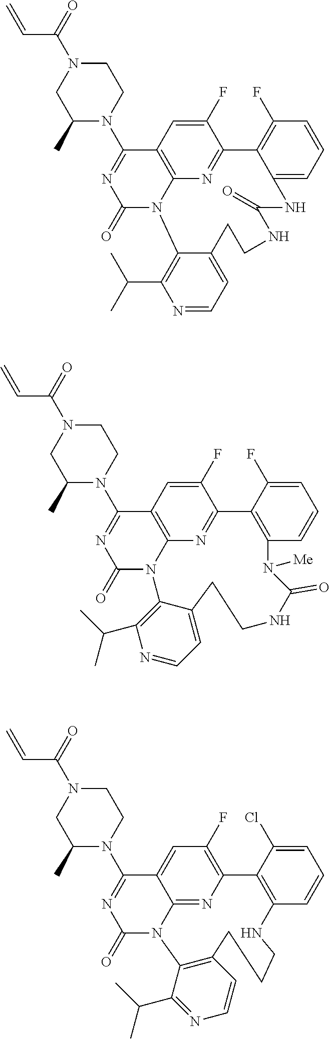 [SMILES: C=CC(=O)N1CCN(c2nc(=O)n3c4nc(c(F)cc24)-c2c(F)cccc2NC(=O)NCCc2ccnc(C(C)C)c2-3)[C@@H](C)C1 ; C=CC(=O)N1CCN(c2nc(=O)n3c4nc(c(F)cc24)-c2c(F)cccc2N(C)C(=O)NCCc2ccnc(C(C)C)c2-3)[C@@H](C)C1 ; C=CC(=O)N1CCN(c2nc(=O)n3c4nc(c(F)cc24)-c2c(Cl)cccc2NCCCc2ccnc(C(C)C)c2-3)[C@@H](C)C1]

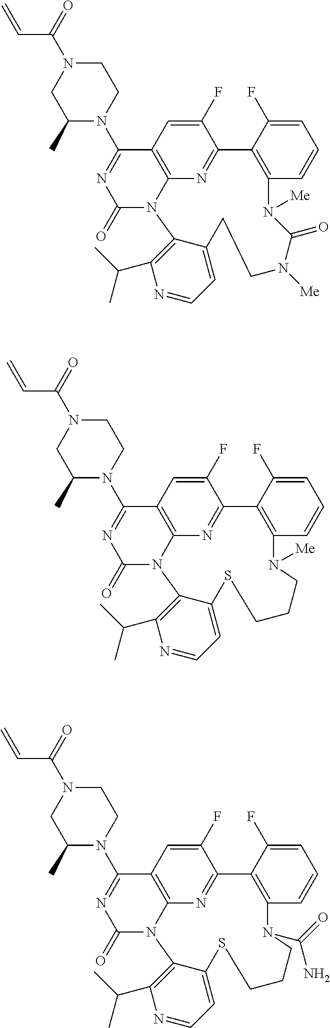 [SMILES: C=CC(=O)N1CCN(c2nc(=O)n3c4nc(c(F)cc24)-c2c(F)cccc2N(C)C(=O)N(C)CCc2ccnc(C(C)C)c2-3)[C@@H](C)C1 ; C=CC(=O)N1CCN(c2nc(=O)n3c4nc(c(F)cc24)-c2c(F)cccc2N(C)CCCSc2ccnc(C(C)C)c2-3)[C@@H](C)C1 ; C=CC(=O)N1CCN(c2nc(=O)n3c4nc(c(F)cc24)-c2c(F)cccc2N(C(N)=O)CCCSc2ccnc(C(C)C)c2-3)[C@@H](C)C1]

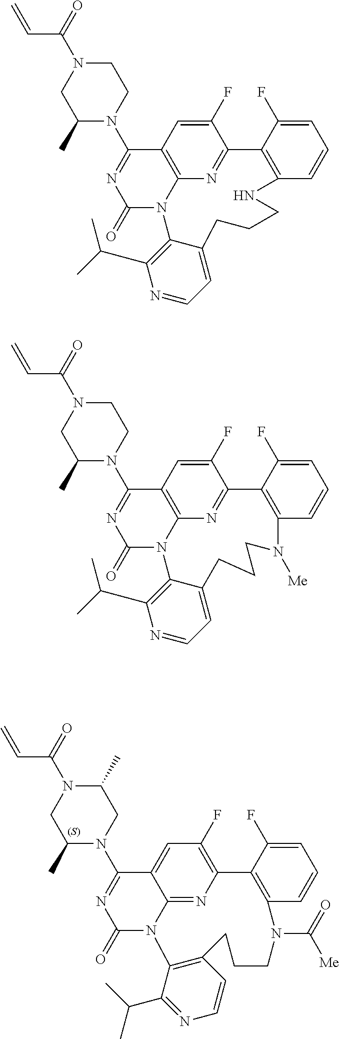

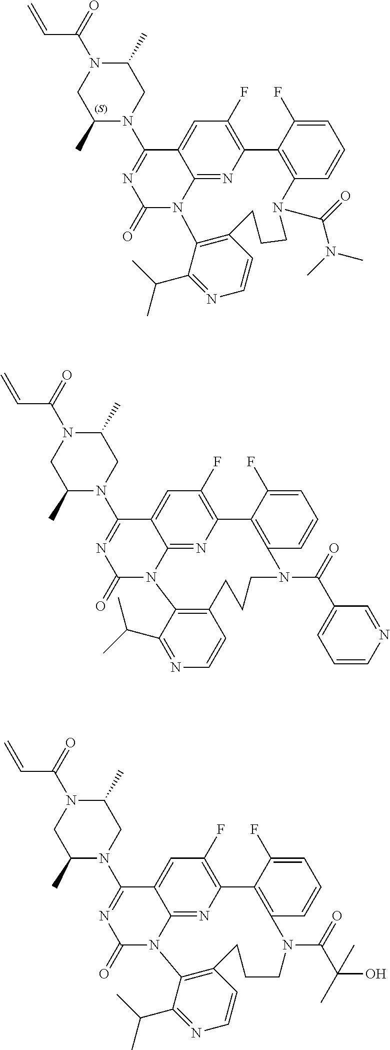

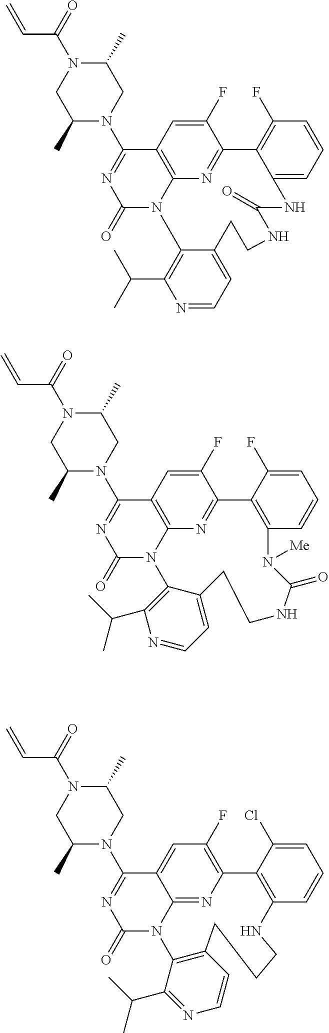 [SMILES: C=CC(=O)N1C[C@H](C)N(c2nc(=O)n3c4nc(c(F)cc24)-c2c(F)cccc2NC(=O)NCCc2ccnc(C(C)C)c2-3)C[C@H]1C ; C=CC(=O)N1C[C@H](C)N(c2nc(=O)n3c4nc(c(F)cc24)-c2c(F)cccc2N(C)C(=O)NCCc2ccnc(C(C)C)c2-3)C[C@H]1C ; C=CC(=O)N1C[C@H](C)N(c2nc(=O)n3c4nc(c(F)cc24)-c2c(Cl)cccc2NCCCc2ccnc(C(C)C)c2-3)C[C@H]1C]

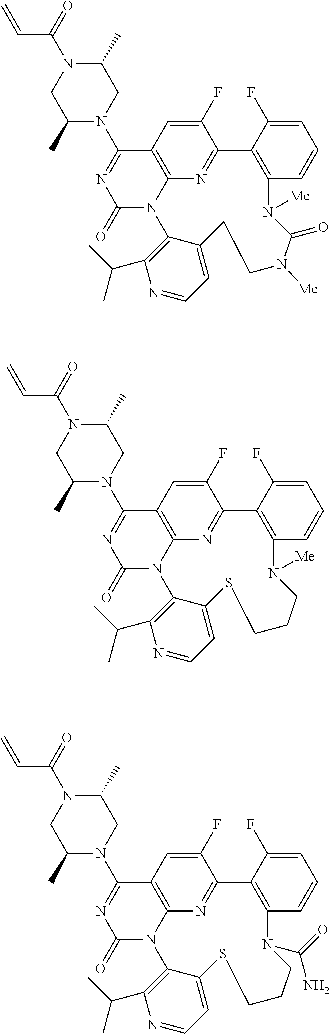 [SMILES: C=CC(=O)N1C[C@H](C)N(c2nc(=O)n3c4nc(c(F)cc24)-c2c(F)cccc2N(C)C(=O)N(C)CCc2ccnc(C(C)C)c2-3)C[C@H]1C ; C=CC(=O)N1C[C@H](C)N(c2nc(=O)n3c4nc(c(F)cc24)-c2c(F)cccc2N(C)CCCSc2ccnc(C(C)C)c2-3)C[C@H]1C ; C=CC(=O)N1C[C@H](C)N(c2nc(=O)n3c4nc(c(F)cc24)-c2c(F)cccc2N(C(N)=O)CCCSc2ccnc(C(C)C)c2-3)C[C@H]1C]

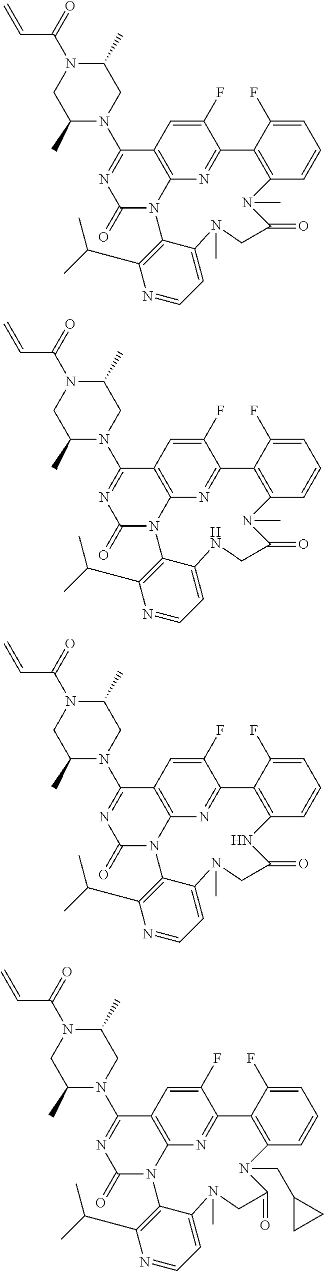 [SMILES: C=CC(=O)N1C[C@H](C)N(c2nc(=O)n3c4nc(c(F)cc24)-c2c(F)cccc2N(C)C(=O)CN(C)c2ccnc(C(C)C)c2-3)C[C@H]1C ; C=CC(=O)N1C[C@H](C)N(c2nc(=O)n3c4nc(c(F)cc24)-c2c(F)cccc2N(C)C(=O)CNc2ccnc(C(C)C)c2-3)C[C@H]1C ; C=CC(=O)N1C[C@H](C)N(c2nc(=O)n3c4nc(c(F)cc24)-c2c(F)cccc2NC(=O)CN(C)c2ccnc(C(C)C)c2-3)C[C@H]1C ; C=CC(=O)N1C[C@H](C)N(c2nc(=O)n3c4nc(c(F)cc24)-c2c(F)cccc2N(CC2CC2)C(=O)CN(C)c2ccnc(C(C)C)c2-3)C[C@H]1C]

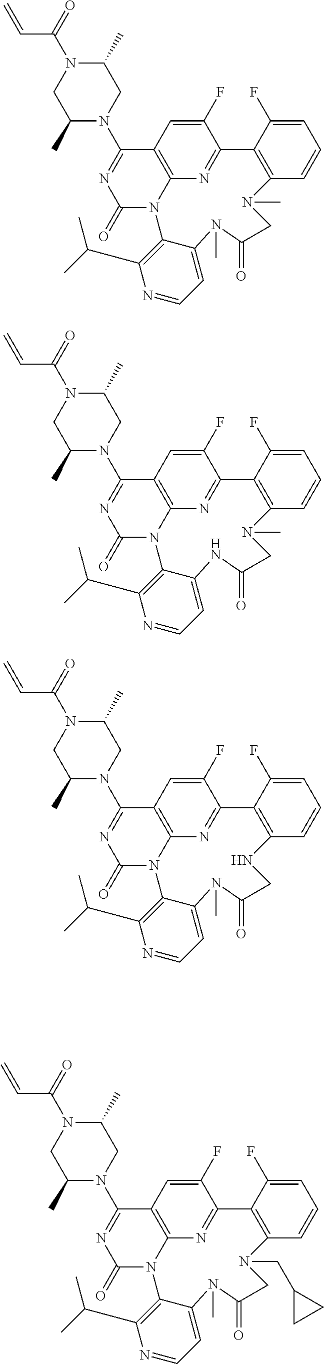 [SMILES: C=CC(=O)N1C[C@H](C)N(c2nc(=O)n3c4nc(c(F)cc24)-c2c(F)cccc2N(C)CC(=O)N(C)c2ccnc(C(C)C)c2-3)C[C@H]1C ; C=CC(=O)N1C[C@H](C)N(c2nc(=O)n3c4nc(c(F)cc24)-c2c(F)cccc2N(C)CC(=O)Nc2ccnc(C(C)C)c2-3)C[C@H]1C ; C=CC(=O)N1C[C@H](C)N(c2nc(=O)n3c4nc(c(F)cc24)-c2c(F)cccc2NCC(=O)N(C)c2ccnc(C(C)C)c2-3)C[C@H]1C ; C=CC(=O)N1C[C@H](C)N(c2nc(=O)n3c4nc(c(F)cc24)-c2c(F)cccc2N(CC2CC2)CC(=O)N(C)c2ccnc(C(C)C)c2-3)C[C@H]1C]

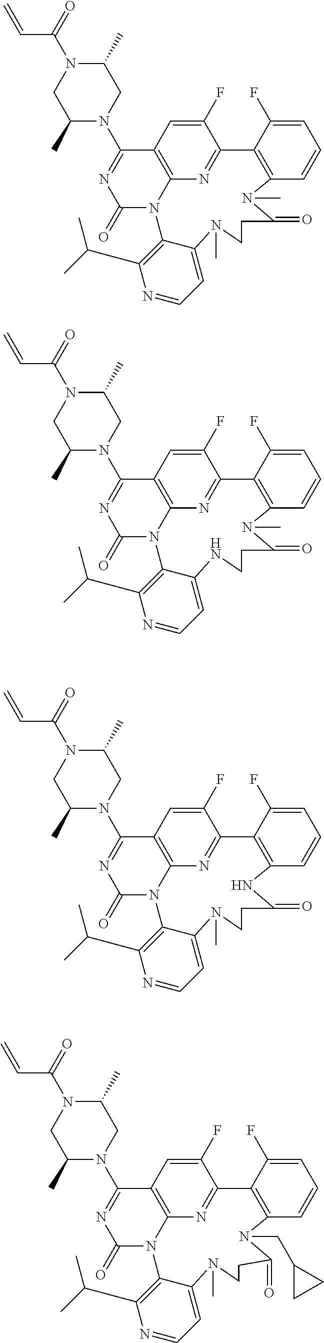 [SMILES: C=CC(=O)N1C[C@H](C)N(c2nc(=O)n3c4nc(c(F)cc24)-c2c(F)cccc2N(C)C(=O)CCN(C)c2ccnc(C(C)C)c2-3)C[C@H]1C ; C=CC(=O)N1C[C@H](C)N(c2nc(=O)n3c4nc(c(F)cc24)-c2c(F)cccc2N(C)C(=O)CCNc2ccnc(C(C)C)c2-3)C[C@H]1C ; C=CC(=O)N1C[C@H](C)N(c2nc(=O)n3c4nc(c(F)cc24)-c2c(F)cccc2NC(=O)CCN(C)c2ccnc(C(C)C)c2-3)C[C@H]1C ; C=CC(=O)N1C[C@H](C)N(c2nc(=O)n3c4nc(c(F)cc24)-c2c(F)cccc2N(CC2CC2)C(=O)CCN(C)c2ccnc(C(C)C)c2-3)C[C@H]1C]

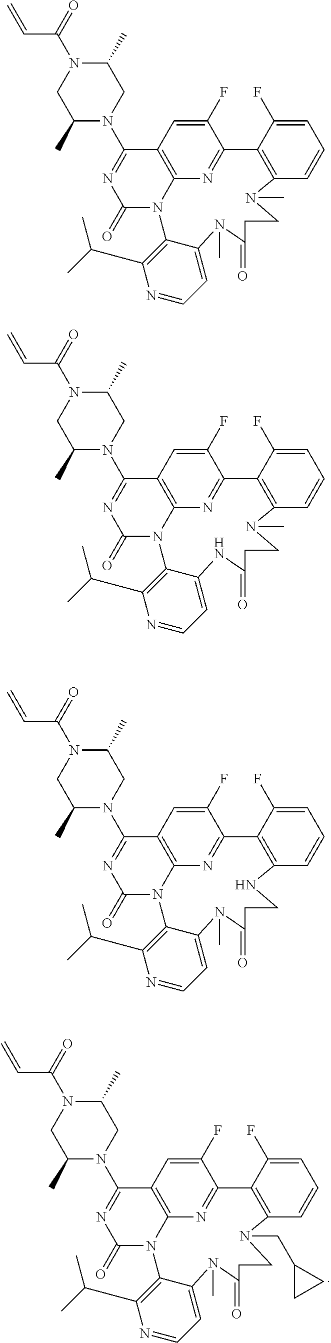 [SMILES: C=CC(=O)N1C[C@H](C)N(c2nc(=O)n3c4nc(c(F)cc24)-c2c(F)cccc2N(C)CCC(=O)N(C)c2ccnc(C(C)C)c2-3)C[C@H]1C ; C=CC(=O)N1C[C@H](C)N(c2nc(=O)n3c4nc(c(F)cc24)-c2c(F)cccc2N(C)CCC(=O)Nc2ccnc(C(C)C)c2-3)C[C@H]1C ; C=CC(=O)N1C[C@H](C)N(c2nc(=O)n3c4nc(c(F)cc24)-c2c(F)cccc2NCCC(=O)N(C)c2ccnc(C(C)C)c2-3)C[C@H]1C ; C=CC(=O)N1C[C@H](C)N(c2nc(=O)n3c4nc(c(F)cc24)-c2c(F)cccc2N(CC2CC2)CCC(=O)N(C)c2ccnc(C(C)C)c2-3)C[C@H]1C]

In another aspect, the invention provided a pharmaceutical composition comprising a pharmaceutically acceptable carrier and a compound disclosed herein or a pharmaceutically acceptable carrier.

In another aspect the invention provided a method for treating a disease mediated by KRAS G12C mutant. The method comprises administering a therapeutically effective amount of a compound disclosed herein to a subject. In another aspect, the invention features a method of treating any of the following conditions by administering a therapeutically effective amount of a compound disclosed herein to a subject: pancreatic cancer, colorectal cancer, hepatocellular carcinoma, breast cancer, ovarian cancer, lung cancer, liver cancer, a sarcoma, or any other forms of cancer.

The invention includes all possible combinations of the embodiments described above and below.

### Definitions

The term “alkyl,” by itself or as part of another substituent, means, unless otherwise stated, a straight (i.e. unbranched) or branched chain, or cyclic hydrocarbon radical, or combination thereof, which may be fully saturated, mono- or polyunsaturated and can include di- and multivalent radicals, having the number of carbon atoms designated (i.e. C₁₋₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl”. The said alkyl is optionally substituted with one or more halogen atom(s).

The term “Halogenated alkyl” means alkyl as defined above wherein one or more hydrogen atoms have been replaced by halogen atoms.

The term “Alkylene” by itself or as part of another substituent means a divalent radical derived from an alkyl, as exemplified, but not limited, by —CH₂CH₂CH₂CH₂—, —CH₂CH═CHCH₂—, —CH₂C≡CCH₂—, —CH₂CH₂CH(CH₂CH₂CH₃)CH₂—. Typically, an alkyl (or alkylene) group has from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The said alkylene is optionally substituted with one or more halogen atom(s).

The term “Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like. The said alkynyl is optionally substituted with one or more halogen atom(s).

The term “Cycloalkyl” means mono- or bicyclic saturated carbocyclic rings, each of which has from 3 to 10 carbon atoms. A “fused analog” of cycloalkyl means a monocyclic ring fused to an aryl or heteroaryl group in which the point of attachment is on the non-aromatic portion. Examples of cycloalkyl and fused analogs thereof include cyclopropyl, cyclobutyl, cyclopentyl, cyclohexyl, cycloheptyl, tetrahydronaphthyl, decahydronaphthyl, indanyl, and the like. The said cycloalkyl is optionally substituted with one or more halogen atom(s).

The term “Alkoxy” means alkoxy groups of a straight or branched having the indicated number of carbon atoms. C₁₋₆alkoxy, for example, includes methoxy, ethoxy, propoxy, isopropoxy, and the like.

The term “Heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The term “cycloalkoxy” means cycloalkyl as defined above bonded to an oxygen atom, such as cyclopropyloxy.

The term “Halogenated alkoxy” means alkoxy as defined above wherein one or more hydrogen atoms have been replaced by halogen atoms.

The term “aryl” means mono- or bicyclic aromatic rings containing only carbon atoms. A “fused analog” of aryl means an aryl group fused to a monocyclic cycloalkyl or monocyclic heterocyclyl group in which the point of attachment is on the aromatic portion. Examples of aryl and fused analogs thereof include phenyl, naphthyl, indanyl, indenyl, tetrahydronaphthyl, 2,3-dihydrobenzofuranyl, dihydrobenzopyranyl, 1,4-benzodioxanyl, and the like.

The term “heteroaryl” means a mono- or bicyclic aromatic ring containing at least one (such 1, 2 or 3) heteroatom selected from N, O and S, with each ring containing 5 to 6 atoms. A “fused analog” of heteroaryl means a heteroaryl group fused to a monocyclic cycloalkyl or monocyclic heterocyclyl group in which the point of attachment is on the aromatic portion. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like.

The said alkyl groups, aryl groups and said heteroaryl groups referred to in the definitions are unsubstituted or are substituted by at least one substituent selected from the group consisting of substituents.

The said substituents are selected from the group consisting of halogen atoms, hydroxyl group, alkyl groups having from 1 to 4 carbon atoms, alkoxy groups having from 1 to 4 carbon atoms, haloalkyl groups having from 1 to 4 carbon atoms, haloalkoxy groups having from 1 to 4 carbon atoms, cyano groups, alkynyl groups having from 2 to 6 carbon atoms, alkanoyl groups having from 1 to 5 carbon atoms, cycloalkyl groups having from 3 to 7 ring atoms, heteroaryl groups, aryl groups, aralkoxy groups having from 7 to 10 carbon atoms, arylcarbonyl groups, two adjacent-x groups are optionally joined together to form an alkylene or an alkenylene chain having 3 or 4 carbon atoms, aminocarbonyl groups, alkenyl groups having from 2 to 5 carbon atoms, alkylthio groups having from 1 to 4 carbon atoms, aminosulfinyl groups, aminosulfonyl groups, hydroxy groups, —SF₅, hydroxyalkyl groups having from 1 to 4 carbon atoms, nitro groups, amino groups, carboxy groups, alkoxycarbonyl groups having from 2 to 5 carbon atoms, alkoxyalkyl groups having from 1 to 4 carbon atoms, alkylsulfonyl groups having from 1 to 4 carbon atoms, alkanoylamino groups having from 1 to 4 carbon atoms, alkanoyl(alkyl)amino groups having from 1 to 6 carbon atoms, alkanoylaminoalkyl groups having from 1 to 6 carbon atoms in both the alkanoyl and alkyl part, alkanoyl(alkyl)aminoalkyl groups having from 1 to 6 carbon atoms in both the alkanoyl and each alkyl part, alkylsulfonylamino groups having from 1 to 4 carbon atoms, mono- or di-alkylaminocarbonyl groups having from 1 to 6 carbon atoms, mono- or di-alkylaminosulfinyl groups having from 1 to 6 carbon atoms, mono- or di alkylaminosulfonyl groups having from 1 to 6 carbon atoms, aminoalkyl groups having from 1 to 4 carbon atoms, mono- or di-alkylamino groups having from 1 to 6 carbon atoms, mono- or di-alkylaminoalkyl groups having from 1 to 6 carbon atoms in each alkyl part, aralkyl groups having from 7 to 10 carbon atoms, heteroarylalkyl groups having from 1 to 4 carbon atoms in the alkyl part, heteroarylalkoxy groups having from 1 to 4 carbon atoms in the alkoxy part and alkylsulfonylamino groups having from 1 to 4 carbon atoms;

The term “heterocyclyl” means mono- or bicyclic saturated rings containing at least one heteroatom selected from N, S and O, each of said ring having from 3 to 10 atoms in which the point of attachment may be carbon or nitrogen. A “fused analog” of heterocyclyl means a monocyclic heterocycle fused to an aryl or heteroaryl group in which the point of attachment is on the non-aromatic portion. Examples of “heterocyclyl” and fused analogs thereof include pyrrolidinyl, piperidinyl, piperazinyl, imidazolidinyl, 2,3-dihydrofuro(2,3-b)pyridyl, benzoxazinyl, tetrahydrohydroquinolinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils).

The terms “halo” or “halogen,” by themselves or as part of another substituent, mean, unless otherwise stated, a fluorine, chlorine, bromine, or iodine atom. Additionally, terms such as “haloalkyl,” or “halogenated alkyl” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrugs may also have improved solubility in pharmaceutical compositions over the parent drug. An example, without limitation, of a prodrug would be a compound of any of Formula I, which is administered as an ester (the “prodrug”) to facilitate transmittal across a cell membrane where water solubility is detrimental to mobility, but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

Optical Isomers-Diastereomers-Atropisomers-Geometric Isomers-Tautomers:

Compounds any of Formula I to Formula XI may contain one or more asymmetric centers/hindered rotation about a single bond, and may thus occur as racemates and racemic mixtures, single enantiomers, single atropisomers, diastereomeric mixtures and individual diastereomers. The present invention is meant to comprehend all such isomeric forms of the compounds of Formula I to Formula XI.

Some of the compounds described herein contain olefinic double bonds, and unless specified otherwise, are meant to include both E and Z geometric isomers.

Some of the compounds of Formula I to Formula XI may contain one or more than one cyclic ring systems and may thus exist in cis- and trans-isomers. The present invention is meant to include all such cis- and trans-isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I to Formula XI.

Compounds of the Formula I to Formula XI may be separated into diastereoisomeric pairs of enantiomers by, for example, HPLC or fractional crystallization from a suitable solvent, for example MeOH or EtOAc or a mixture thereof. The pair of enantiomers thus obtained may be separated into individual stereoisomers by conventional means, for example by the use of an optically active amine or acid as a resolving agent or on a chiral HPLC column.

Alternatively, any enantiomer of a compound of the general Formula I to Formula XI may be obtained by stereospecific synthesis using optically pure starting materials or reagents of known configuration.

Stable Isotope-Labeled Analogs:

One or more than one of the protons in compounds of Formula I to Formula XI can be replaced with deuterium atom(s), thus providing deuterated analogs that may have improved pharmacological activities.

Salts and Formulations

The compounds described herein can be useful as the free base or as a salt.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydramine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is alkaline, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I are meant to also include the pharmaceutically acceptable salts.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients are mixed with water or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethyl-cellulose, methylcellulose, hydroxypropylmethy-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an I atomizer using electrohydrodynamics to produce a fine mist), or nebulizer, with or without the use of a suitable propellant, such as 1, 1,1,2-tetrafluoroethane or 1,1,1,2,3,3,3-heptafluoropropane. For intranasal use, the powder may comprise a bioadhesive agent, for example, chitosan or cyclodextrin.

The pressurized container, pump, spray, atomizer, or nebulizer contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 microns).

This may be achieved by any appropriate comminuting method, such as spiral jet milling, fluid bed jet milling, supercritical fluid processing to form nanoparticles, high pressure homogenization, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomizer using electrohydrodynamics to produce a fine mist may contain from log to 20 mg of the compound of the invention per actuation and the actuation volume may vary from 11 to 1001. A typical formulation may comprise a compound of Formula I to XI propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 fig to 10 mg of the compound of Formula I to XI. The overall daily dose will typically be in the range 1 lag to 10 mg which may be administered in a single dose or, more usually, as divided doses throughout the day.

Compounds of Formula I to XI may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compound of Formula I to Formula XI are employed. (For purposes of this application, topical application shall include mouth washes and gargles.)

Dosage levels of the order of from about 0.01 mg to about 140 mg/kg of body weight per day are useful in the treatment of the above-indicated conditions, or alternatively about 0.5 mg to about 7 g per patient per day. For example, a condition may be effectively treated by the administration of from about 0.01 to 50 mg of the compound per kilogram of body weight per day, or alternatively about 0.5 mg to about 3.5 g per patient per day, preferably 2.5 mg to 1 g per patient per day.

The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition.

Dosage unit forms will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

Indications

Compounds of the present invention may be used to treat diseases with KRAS G12C mutant and the disease is any forms of cancer.

Combination and Targeted Therapy

Administration of the KRAS G12C mutant inhibitors disclosed herein can be combined with other cancer treatments. For example, the inhibitors can be administered in combination with surgical treatments, radiation, or other therapeutic agents such as antibodies, other kinase inhibitors, a target therapy, an inhibitor of MAP kinase signaling pathway, or chemotherapeutics. The inhibitors may also be administered in combination with RNAi therapy, antisense therapy, or immunotherapies. The KRAS G12C mutant inhibitors described herein may be combined with one, two, or more other therapeutic agents. In the examples outlined below, it is understood that “second therapeutic agent” also includes more than one therapeutic agent other than the KRAS G12C mutant inhibitor. For instance, the compounds disclosed herein may be combined with an agent such as sorafenib, a PD-1 antibody or a PD-L1 antibody. A KRAS G12C mutant inhibitor described herein may be administered with one, two, or more other therapeutic agents.

Synthesis

The compounds of the present invention can be prepared according to the following synthetic schemes:

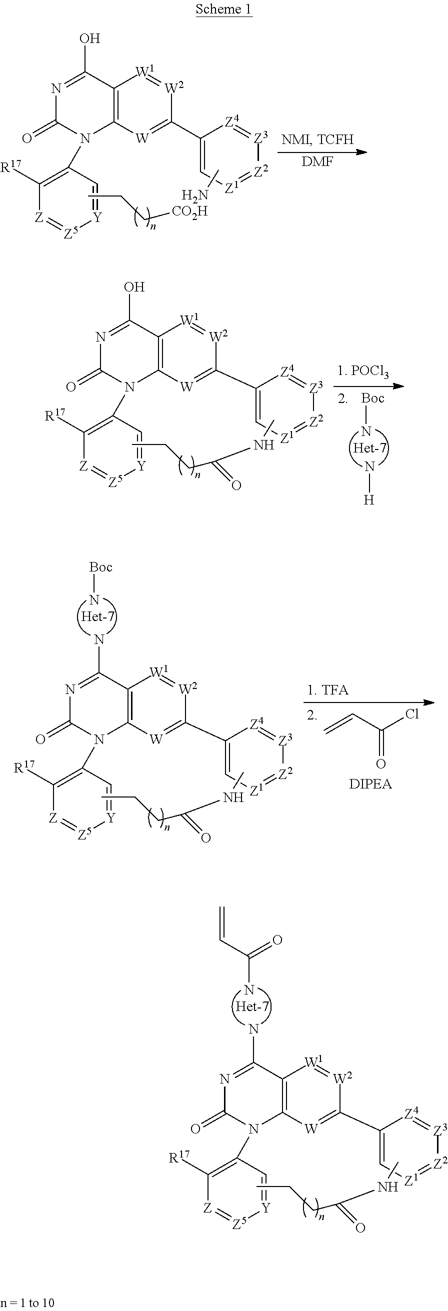

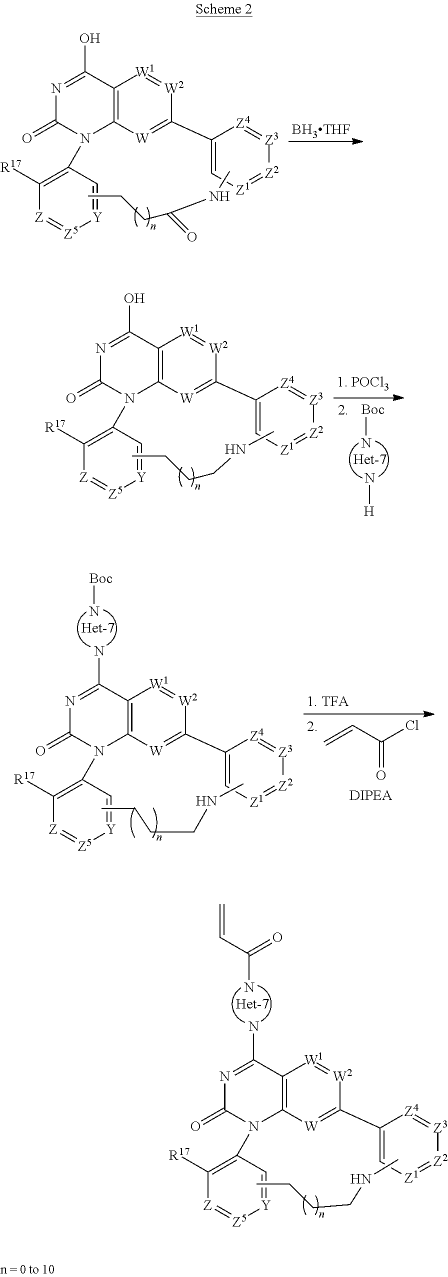

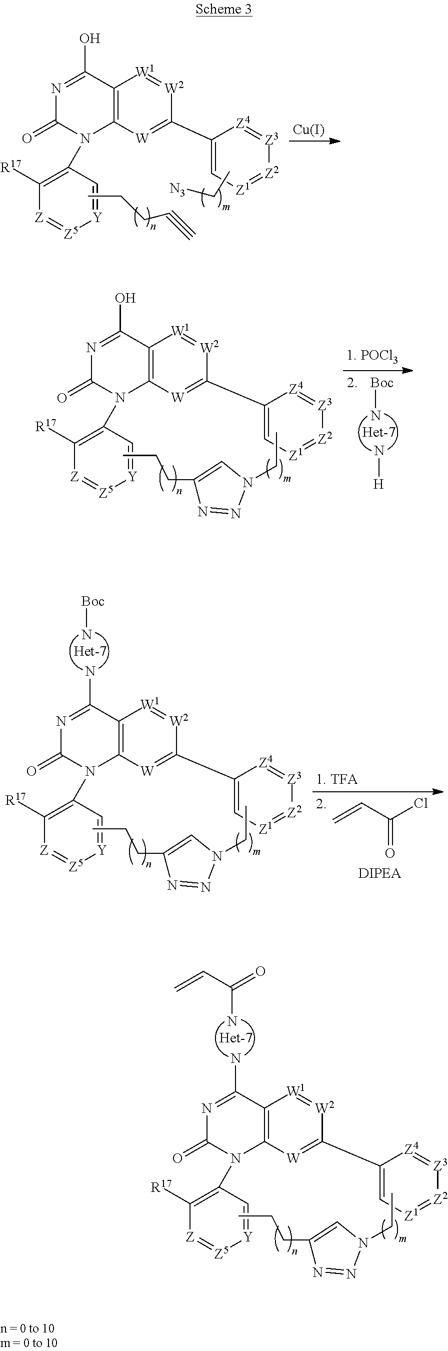

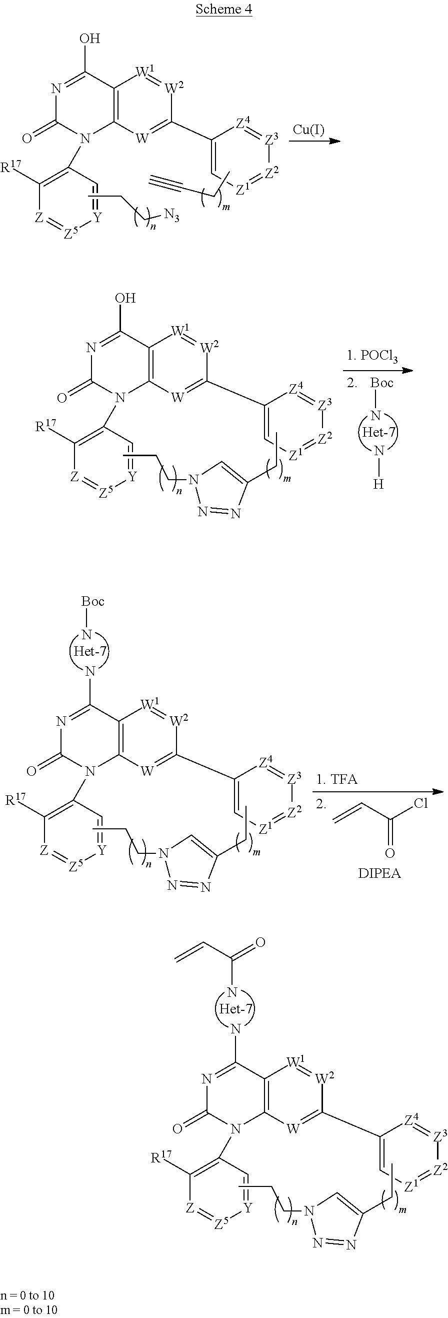

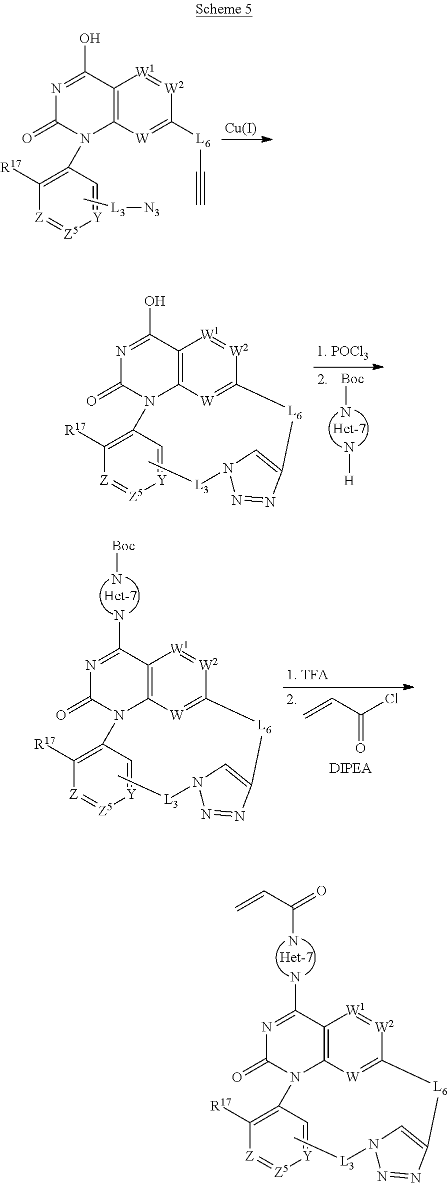

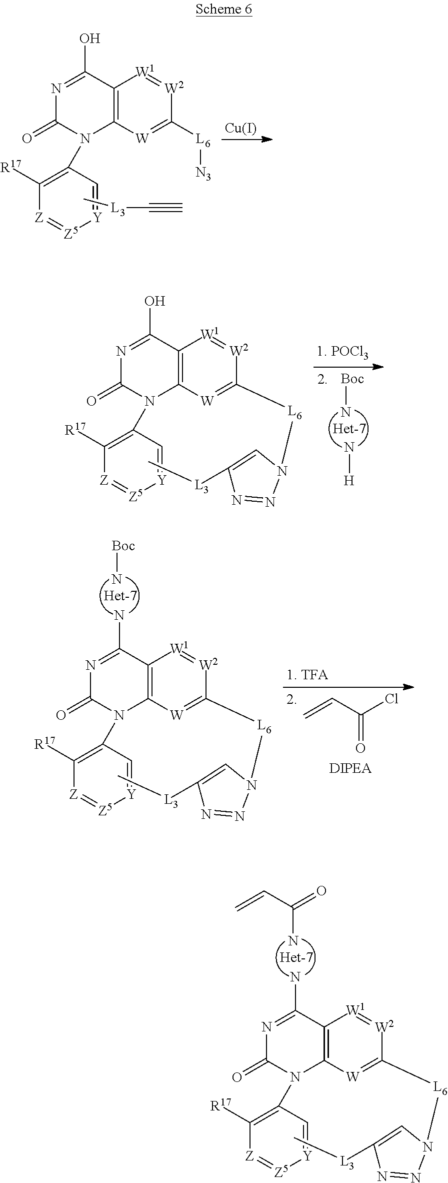

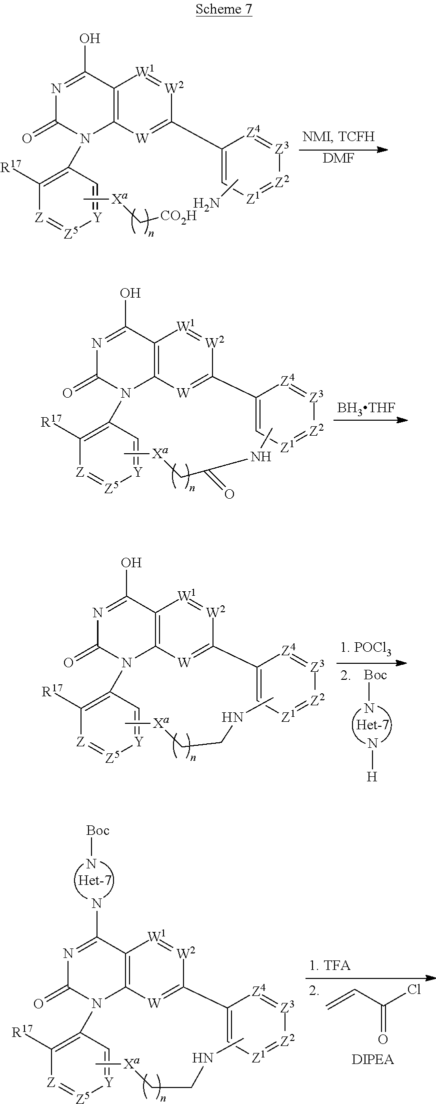

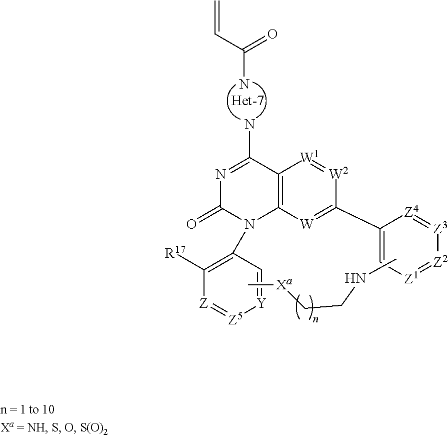

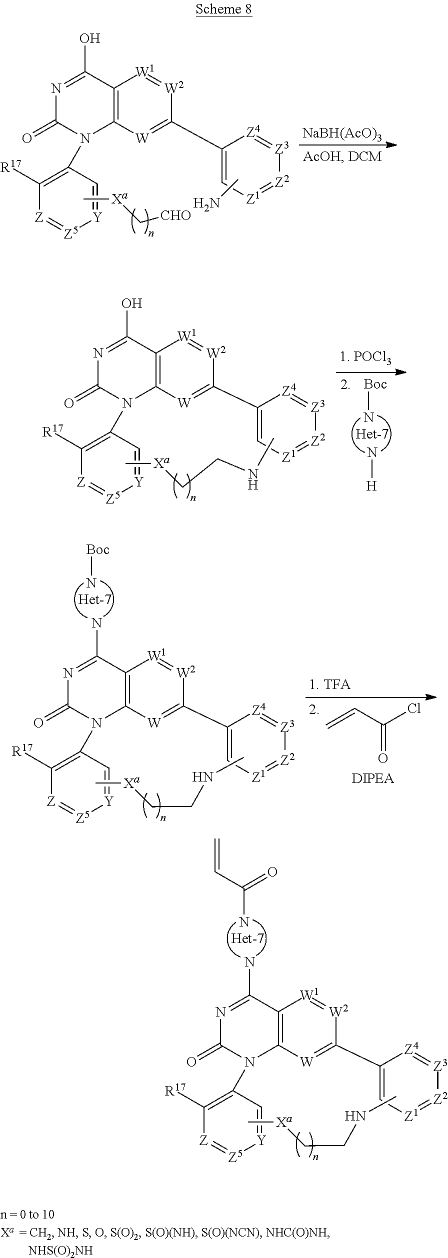

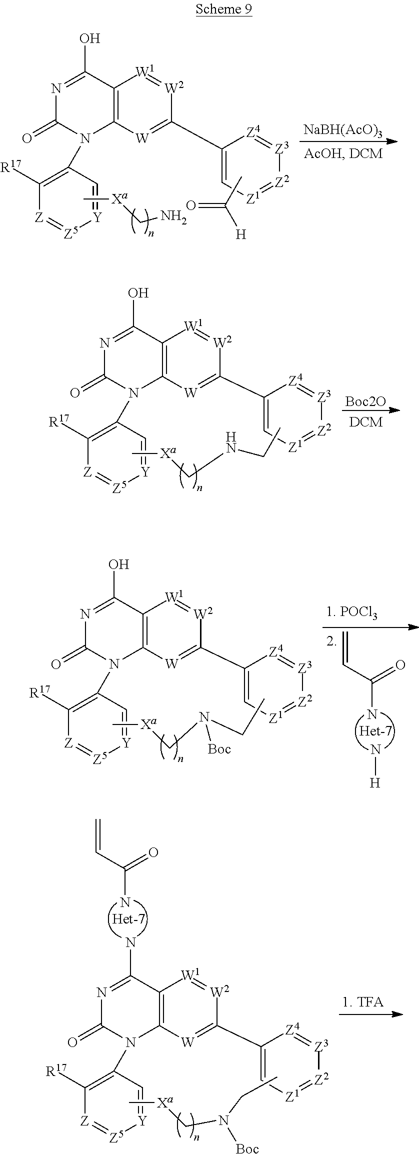

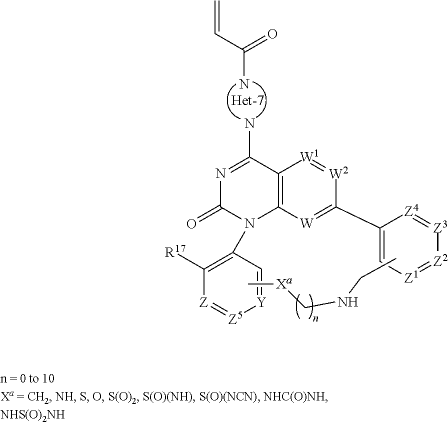

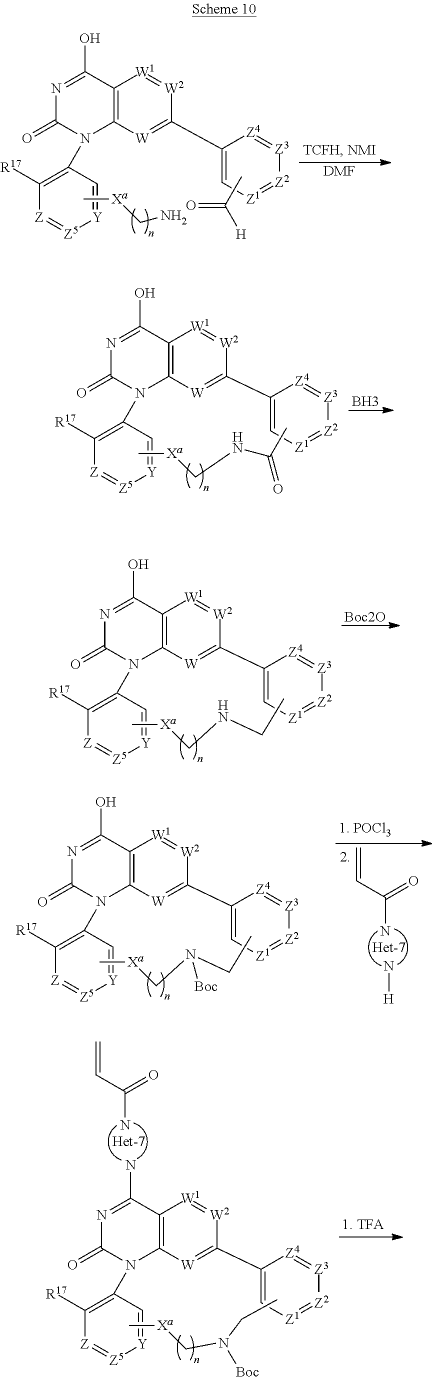

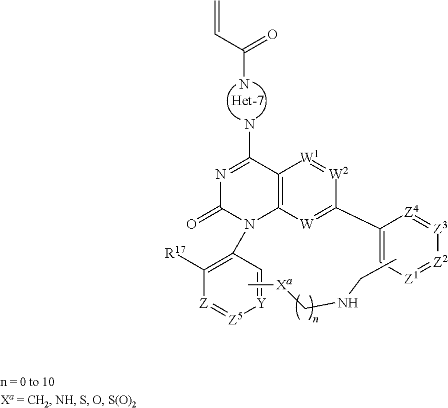

Evaluation of Biological Activity

3D proliferation assay: NCI-H358 (H358, KRAS G12C) and LS513 (KRAS G12D) cancer cell lines were obtained from ATCC (American Type Culture Collection, VA). Cells were plated in 96-well spheroid plate (CORNING INC, NY)) in RPMI-1640 with 10% FBS. Compounds (11-point dilutions) and DMSO were added to the wells and incubated with cells for 4 days at 37° C. Cell viability was then determined by CellTiter-Glo (Promega, WI). IC₅₀ values of compounds were determined as the concentration of 50% inhibition of cell viability compared to DMSO treated cells (A: IC₅₀<0.1 μM; B: IC₅₀ between 0.1 μM and 1 μM; C: IC₅₀ between 1 μM and 10 μM; D: >10 μM; ND: not determined).

Phospho-ERK (pERK) assay: NCI-H358 cells were seeded in 96-well plates (Greiner) in RPMI-1640 with 10% FBS 16 hours prior to compound treatment. Serial dilution of compounds was made and added to wells, and then incubated at 37° C. for 3 hours. After the treatment, cells were fixed with 3.7% formaldehyde (VWR) for 20 min at RT, followed by being permeabilized with ice-cold methanol at −20° C. for 20 min. The methanol was then dumped and replaced with Intercept® (PBS) Blocking Buffer (LiCOR) supplemented with 0.05% Tween-20 and incubated at RT for 1 hour with gentle rocking. The blocking buffer was then replaced with the blocking buffer containing pERK1/2 antibody (Cell Signaling) and incubated at 4° C. overnight with gentle rocking. The plate was washed 5 times with 1×PBS+0.1% Tween-20. The blocking buffer containing LiCOR IRDye 680RD secondary antibody (LiCOR,) was then added and incubated for 1 hour at RT with gentle rocking. After washing 5 times with 1×PBS+0.1% Tween-20, the plate was read using CLARIOstar plate reader (BMG LABTECH GmbH). IC₅₀ values were determined as the concentration of 50% inhibition of the fluorescence signal compared to DMSO treated cells (A: IC₅₀<0.1 μM; B: IC₅₀ between 0.1 μM and 1 μM; C: IC₅₀ between 1 μM and 10 μM; D: >10 μM; ND: not determined).

IC₅₀ of examples

| | 3D proliferation IC₅₀ | | pERK IC₅₀ |
|---|---|---|---|
| Compound | H358 | LS513 | H358 |
| Example 1 | A | D | B |
| Example 2 | A | D | ND |
| Example 2A | A | D | A |
| Example 2B | B | D | B |
| Example 3 | C | ND | ND |
| Example 4 | A | D | A |
| Example 5 | B | ND | ND |
| Example 6A | D | ND | ND |
| Example 6B | B | D | ND |
| Example 7A | B | D | B |
| Example 7B | A | D | A |
| Example 8 | A | D | A |
| Example 9 | A | D | A |
| Example 10 | A | D | A |
| Example 11 | B | D | ND |
| Example 12 | B | D | ND |
| Example 13 | B | ND | |
| Example 14 | C | ND | ND |
| Example 15 | B | D | B |
| Example 16 | C | ND | ND |
| Example 17 | A | C | B |
| Example 18 | B | D | ND |
| Example 19 | A | C | A |
| Example 20 | A | D | A |
| Example 20A | ND | ND | ND |
| Example 20B | ND | ND | ND |
| Example 21 | A | D | ND |
| Example 22 | A | B | B |
| Example 23A | A | D | ND |
| Example 23B | B | C | ND |
| Example 24A | A | D | ND |
| Example 24B | A | D | ND |
| Example 25 | A | D | ND |
| Example 26 | A | D | ND |
| Example 27A | ND | ND | ND |
| Example 27B | ND | ND | ND |
| Example 28 | A | D | B |
| Example 29 | A | D | ND |
| Example 30 | A | D | ND |
| Example 31 | ND | ND | ND |

The following abbreviations have the meanings indicated. CIP means 2-chloro-1,3-dimethylimidazolidinium hexafluorophosphate; EA means ethyl acetate; DBU means 1,8-diazabicyclo[5.4.0]undec-7-ene; DIBAL means diisobutyl aluminum hydride; DIPEA means diisopropyl ethyl amine; DMAP means N,N-dimethylaminopyridine; DME means 1,2-dimethoxyethane; DMF means N,N-dimethylformamide; dmpe means 1,2-bis(dimethylphosphino)ethane; DMSO means dimethylsulfoxide; dppb means 1,4-bis(diphenylphosphino)butane; dppe means 1,2-bis(diphenylphosphino)ethane; dppf means 1,1′-bis(diphenylphosphino)ferrocene; dppm means 1,1′-bis(diphenylphosphino)methane; DIAD means diisopropylazodicarboxylate; EDCI means 1-(3-dimethylaminopropyl)-3-ethylcarbodiimide; HATU means 2-(7-Aza-1H-benzotriazole-1-yl)-1,1,3,3-tetramethyluronium hexafluorophosphate; HMPA means hexamethylphosphorarnide; IPA means isopropyl alcohol; LDA means lithium diisopropylamide; LHMDS means lithium bis(hexamethyldisilylamide); LAH means lithium aluminum hydride; NCS means N-chlorosuccinimide; PE means petroleum ether; PyBOP means benzotriazol-1-yloxytripyrrolidinophosphonium hexafluorophosphate; TDA means tris(2-(2-methoxyethoxy)ethyl)amine; DCM means dichloromethame; TEA means triethylamine; TFA means trifluoroacetic acid; THF means tetrahydrofuran; NCS means N-chlorosuccinimide; NMM means N-methylmorpholine; NMP means N-methylpyrrolidine; NMM means N-methylmorpholine; NMI means M-methylimidazole; PPh3 means triphenylphosphine, RT or rt means room temperature; STAB means sodium triacetoxyboronhydride; TCFH means Chloro-N,N,N′,N′-tetramethylformamidinium hexafluorophosphate; T3P means propylphosphonic anhydride.

HPLC-MS analyses were performed on Waters HPLC 2790 with Waters micromass ZQ 4000 (Model MAA050) as mass detector and Waters 2487 UV as detector. Column used was Phenomemex OOB-4605-EO (5 u-XB-C18-100A, 50×4.6 mm). The mobile phase consists eluent A (water, 0.05% TFA) and eluent B (CH₃CN, 0.05% TFA), and the elution proceeded at 1 mL/min. The initial conditions were 90% A for 1 min, then 90% A to 10% A linearly decreased within 5 min, then from 10% A to 90% A within 1 min. The total run time is 7 minutes.

The present invention will be more readily understood by referring to the following examples which are given to illustrate the invention rather than to limit its scope.

### EXAMPLE 1

### (S)-2⁴-(4-Acryloyl-2-methylpiperazin-1-yl)-2⁶,3⁶-difluoro-1²-isopropyl-2¹,2²-dihydro-4-aza-2(1,7)-pyrido[2,3-d]pyrimidina-1(3,4)-pyridina-3(1,2)-benzenacycloheptaphane-2²,5-dione

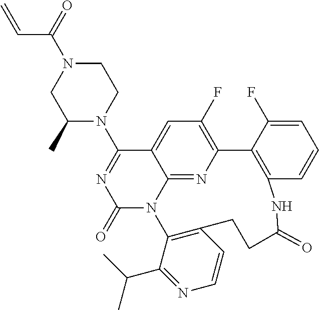 [SMILES: C=CC(=O)N1CCN(c2nc(=O)n3c4nc(c(F)cc24)-c2c(F)cccc2NC(=O)CCc2ccnc(C(C)C)c2-3)[C@@H](C)C1]

### Step 1 tert-butyl (E)-3-(3-amino-2-isopropylpyridin-4-yl)acrylate

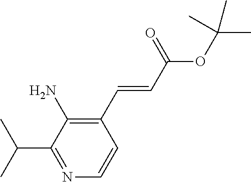 [SMILES: CC(C)c1nccc(/C=C/C(=O)OC(C)(C)C)c1N]

To a stirred solution of 4-iodo-2-isopropylpyridin-3-amine (690 mg, 2.63 mmol), tert-butyl acrylate (505 mg, 3.95 mmol), tri-tolylphosphine (79 mg, 0.26 mmol) and TEA (399 mg, 3.95 mmol) in DMF (10 ml) was added Pd(OAc)₂ (58 mg, 0.26 mmol) under Ar. The resulting mixture was stirred at 100° C. for 3 h to give a black suspension. Water (20 mL) and EtOAc (50 mL) were then added. The organic layer was washed with brine, dried over Na₂SO₄, filtered and concentrated. The residue was purified by silica gel chromatography (PE:EtOAc=1:1) to give 600 mg of the title product as a yellow solid. MS (ES+): 262.8[M+1]⁺.

### Step 2 tert-butyl 3-(3-amino-2-isopropylpyridin-4-yl)propanoate

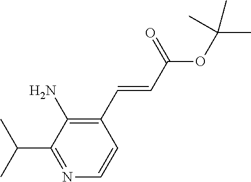 [SMILES: CC(C)c1nccc(/C=C/C(=O)OC(C)(C)C)c1N]

To a stirred solution of tert-butyl (E)-3-(3-amino-2-isopropylpyridin-4-yl)acrylate (600 mg, 2.3 mmol) in MeOH (10 ml) was added 10% Pd/C (100 mg) at room temperature. The reaction vessel was purged three times with H₂ and the resulting mixture was stirred at 30° C. for 16 h. The mixture was then filtered and the filtrate was concentrated to give the title product as yellow oil. MS (ES+): 264.8[M+1]⁺.

### Step 3 tert-butyl 3-(3-(3-(2,6-dichloro-5-fluoronicotinoyl)ureido)-2-isopropylpyridin-4-yl)propanoate

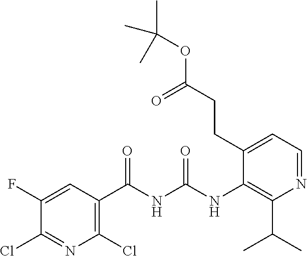 [SMILES: CC(C)c1nccc(CCC(=O)OC(C)(C)C)c1NC(=O)NC(=O)c1cc(F)c(Cl)nc1Cl]

To a stirred solution of 2,6-dichloro-5-fluoronicotinamide (157 mg, 0.754 mmol) in THF (5 ml) was added oxalyl chloride (192 mg, 1.49 mmol) at room temperature under Ar. After the resulting mixture was stirred for 1 h at 80° C., the solvent was removed under reduced pressure. The residue was then diluted with 5 ml of THF and was added dropwise to a stirred solution of the product of Step 2 (100 mg, 0.378 mmol) at 0° C. After stirring for 1 h at 0° C., the reaction mixture was quenched with water and extracted with EtOAc. The organic layer was washed with brine, dried over Na₂SO₄, filtered and concentrated. The residue was purified by Prep-TLC (PE:EtOAc=1:1) to give 168 mg of the title product as a white solid. MS (ES+): 499.0[M+1]⁺.

### Step 4 tert-butyl 3-(3-(7-chloro-6-fluoro-4-hydroxy-2-oxopyrido[2,3-d]pyrimidin-1(2H)-yl)-2-isopropylpyridin-4-yl)propanoate

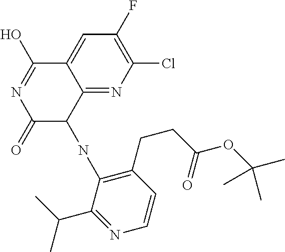 [SMILES: CC(C)c1nccc(CCC(=O)OC(C)(C)C)c1NC1C(=O)N=C(O)c2cc(F)c(Cl)nc21]

To a stirred solution of the product of Step 3 (791 mg, 1.58 mmol) in THF (12 ml) was added KHMDS (1.0 M) (3.5 ml, 3.49 mmol) at room temperature under Ar. After stirring for 1 h at room temperature, the reaction was quenched with water and extracted with ethyl acetate. The organic layer was washed with brine, dried over Na₂SO₄, filtered and concentrated to dryness to give 671 mg of pure product as a white solid. MS (ES+): 463.0[M+1]⁺.

### Step 5 tert-butyl 3-(3-(7-(2-amino-6-fluorophenyl)-6-fluoro-4-hydroxy-2-oxopyrido[2,3-d]pyrimidin-1(2H)-yl)-2-isopropylpyridin-4-yl)propanoate

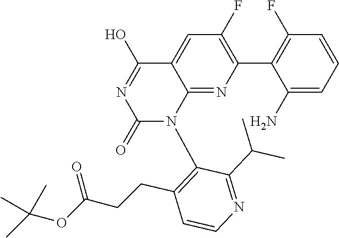 [SMILES: CC(C)c1nccc(CCC(=O)OC(C)(C)C)c1-n1c(=O)nc(O)c2cc(F)c(-c3c(N)cccc3F)nc21]

To a stirred solution the product of Step 4 (210 mg, 0.453 mmol), 3-fluoro-2-(4,4,5,5-tetramethyl-1,3,2-dioxaborolan-2-yl)aniline (215 mg, 0.907 mmol) and KOAc (134 mg, 1.36 mmol) in dioxane (6 ml) and H₂O (2 drops) was added Pd(dppf)Cl₂.DCM (37 mg, 0.045 mmol) at room temperature under Ar. After stirring for 1.5 h at 80° C. to give a blank solution, the reaction mixture treated with water and extracted with EtOAc. The organic layer was washed with brine, dried over Na₂SO₄, filtered and concentrated. The residue was purified by silica gel chromatography (PE:EtOAc=1:2) to give 229 mg of the title product as a white solid. MS (ES+): 537.8[M+1]⁺.

### Step 6 3-(3-(7-(2-amino-6-fluorophenyl)-6-fluoro-4-hydroxy-2-oxopyrido[2,3-d]pyrimidin-1(2H)-yl)-2-isopropylpyridin-4-yl)propanoic acid

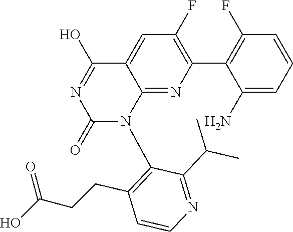 [SMILES: CC(C)c1nccc(CCC(=O)O)c1-n1c(=O)nc(O)c2cc(F)c(-c3c(N)cccc3F)nc21]

To a stirred solution of the product of Step 5 (229 mg, 0.426 mmol) in DCM (3 ml) was added TFA (1 ml) at room temperature under Ar. After stirring for 3 h at 25° C., the reaction mixture was concentrated to dryness to give 360 mg of the title product as yellow oil. MS (ES+): 482.0[M+1]⁺.

### Step 7 2⁶,3⁶-difluoro-2⁴-hydroxy-1²-isopropyl-2¹,2²-dihydro-4-aza-2(1,7)-pyrido[2,3-d]pyrimidina-1(3,4)-pyridina-3(1,2)-benzenacycloheptaphane-2²,5-dione

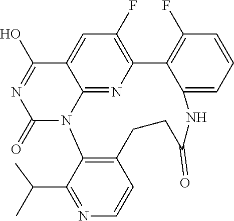 [SMILES: CC(C)c1nccc2c1-n1c(=O)nc(O)c3cc(F)c(nc31)-c1c(F)cccc1NC(=O)CC2]

To a stirred solution of the product of Step 6 (360 mg, 0.74 mmol) and NMI (920 mg, 14.5 mmol) in DMF (20 ml) was added TCFH (628 mg, 3.74 mmol) at room temperature under Ar. The resulting mixture was stirred for 1 h at 25° C. and then quenched with water and extracted with EtOAc. The organic layer was washed with brine, dried over Na₂SO₄, filtered and concentrated. The residue was purified by silica gel chromatography (DCM:MeOH=10:1) to give 160 mg of the title product as a yellow solid. MS (ES+): 463.8[M+1]⁺

### Step 8 tert-butyl (S)-4-(2⁶,3⁶-difluoro-1²-isopropyl-2²,5-dioxo-2¹,2²-dihydro-4-aza-2(1,7)-pyrido[2,3-d]pyrimidina-1(3,4)-pyridina-3(1,2)-benzenacycloheptaphane-2⁴-yl)-3-methylpiperazine-1-carboxylate

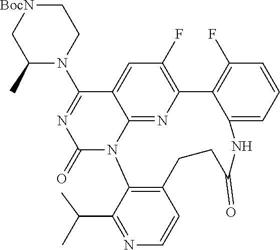 [SMILES: CC(C)c1nccc2c1-n1c(=O)nc(N3CCN(C(=O)OC(C)(C)C)C[C@@H]3C)c3cc(F)c(nc31)-c1c(F)cccc1NC(=O)CC2]

To a stirred solution of the product of Step 8 (160 mg, 0.344 mmol) and DIPEA (446 mg, 3.44 mmol) in CH₃CN (3 ml) was added POCl₃ (318 mg, 2.06 mmol) at room temperature under Ar. The resulting mixture was stirred for 1 h at 80° C. and then concentrated to dryness. The residue was dissolved in 3 ml of DMF and the resulted solution was treated with DIPEA (244 mg, 1.9 mmol) and tert-butyl (S)-3-methylpiperazine-1-carboxylate (140 mg, 0.68 mmol) at room temperature under Ar. After stirring for 2 h at 25° C., the reaction was quenched with water and extracted with EtOAc. The organic layer was washed with brine, dried over Na₂SO₄, filtered and concentrated. The residue was purified by Prep-TLC (EtOAc:MeOH=15:1) to give 33 mg of the title product as a yellow solid. MS (ES+): 646.0[M+1]⁺.

### Step 9 (S)-2⁴-(4-acryloyl-2-methylpiperazin-1-yl)-2⁶,3⁶-difluoro-1²-isopropyl-2¹,2²-dihydro-4-aza-2(1,7)-pyrido[2,3-d]pyrimidina-1(3,4)-pyridina-3(1,2)-benzenacycloheptaphane-2²,5-dione

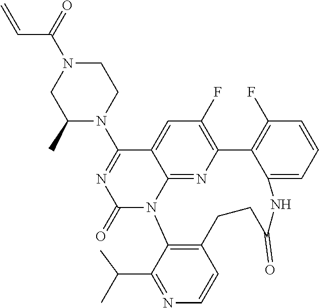 [SMILES: C=CC(=O)N1CCN(c2nc(=O)n3c4nc(c(F)cc24)-c2c(F)cccc2NC(=O)CCc2ccnc(C(C)C)c2-3)[C@@H](C)C1]

To a stirred solution of the product of Step 8 (30 mg, 0.046 mmol) in DCM (2 ml) was added TFA (0.5 ml) at room temperature under Ar. After stirring for 1 h at 25° C., the mixture was concentrated to dryness and diluted with 2 ml of DCM. The resulting solution was treated with DIPEA (24 mg, 0.186 mmol) and acryloyl chloride (5 mg, 0.046 mmol) at room temperature under Ar. The resulted mixture was stirred for 0.5 h at 25° C., the reaction was quenched with water and extracted with EtOAc. The organic layer was washed with brine, dried over Na₂SO₄, filtered and concentrated. The residue was purified by Prep-TLC (EtOAc:MeOH=15:1) to give 5.5 mg of the title product as a yellow solid. MS (ES+): 600.0[M+1]⁺.

¹HNMR Spectrum: (400 MHz, CD₃OD) δ 8.43 (d, 1H), 8.24˜8.21 (m, 1H), 7.42˜7.33 (m, 2H), 7.00˜6.96 (m, 2H), 6.92˜6.90 (m, 1H), 6.2 (d, 1H), 5.72 (d, 1H), 5.22 (m, 1H), 3.98 (m, 3H), 3.52˜3.51 (m, 1H), 2.9 (m, 1H), 2.74˜2.67 (m, 2H), 2.35 (m, 1H), 2.14˜2.06 (m, 1H), 1.9 (m, 1H), 1.3˜1.1 (m, 6H), 0.36˜0.25 (m, 2H).

### EXAMPLE 2

### (S)-2⁴-(4-acryloyl-2-methylpiperazin-1-yl)-2⁶,3⁶-difluoro-1²-isopropyl-2¹,2²-dihydro-4-aza-2(1,7)-pyrido[2,3-d]pyrimidina-1(3,4)-pyridina-3(1,2)-benzenacycloheptaphan-2²-one

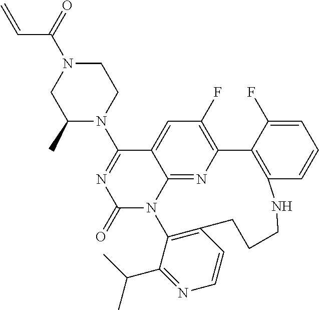 [SMILES: C=CC(=O)N1CCN(c2nc(=O)n3c4nc(c(F)cc24)-c2c(F)cccc2NCCCc2ccnc(C(C)C)c2-3)[C@@H](C)C1]

### Step 1 2⁶,3⁶-difluoro-2⁴-hydroxy-1²-isopropyl-2¹,2²-dihydro-4-aza-2(1,7)-pyrido[2,3-d]pyrimidina-1(3,4)-pyridina-3(1,2)-benzenacycloheptaphan-2²-one

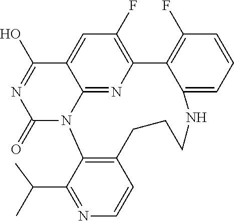 [SMILES: CC(C)c1nccc2c1-n1c(=O)nc(O)c3cc(F)c(nc31)-c1c(F)cccc1NCCC2]

To a stirred solution of the product of Step 7 of Example 1 (6.7 g, 14.5 mmol) in DME (140 ml) was added Borane-methyl sulfide complex (10.0 M, 5.78 ml, 5.78 mmol) at 0° C. under Ar. The reaction mixture was stirred for 2 h at 45° C. and then quenched with MeOH at 0° C. After stirring for 2 h, water was added and the reaction mixture extracted with EtOAc. The organic was washed with brine, dried over Na₂SO₄, filtered and concentrated. The residue was purified by silica gel chromatography (EA:PE=4:1) to give 3.5 g of pure product. MS (ES+): 450.0 [M+1]⁺.

### Step 2 tert-butyl (S)-4-(2⁶,3⁶-difluoro-1²-isopropyl-2²-oxo-2¹,2²-dihydro-4-aza-2(1,7)-pyrido[2,3-d]pyrimidina-1(3,4)-pyridina-3(1,2)-benzenacycloheptaphane-2⁴-yl)-3-methylpiperazine-1-carboxylate

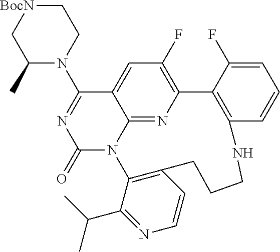 [SMILES: CC(C)c1nccc2c1-n1c(=O)nc(N3CCN(C(=O)OC(C)(C)C)C[C@@H]3C)c3cc(F)c(nc31)-c1c(F)cccc1NCCC2]

To a stirred solution of the product of Step 1 (2 g, 4.3 mmol) and DIPEA (5.74 g, 44.5 mmol) in CH₃CN (20 ml) was added POCl₃ (4.09 g, 26.7 mmol) at rt under Ar. The mixture was stirred for 0.5 h at 80° C., and then was concentrated to dryness. The residue was dissolved in 20 ml of DMF and the resulting solution was treated with DIPEA (2.87 g, 22.3 mmol) and tert-butyl (S)-3-methylpiperazine-1-carboxylate (1.78 g, 8.9 mmol) at 0° C. After stirring for 5 min at 0, the reaction was quenched with water and extracted with EtOAc. The organic layer was washed with brine, dried over Na₂SO₄, filtered and concentrated. The residue was purified by silica gel column chromatography (EtOAc:MeOH=15:1) to give 1.54 g of the title product as a yellow solid. MS (ES+): 632.0 [M+1]⁺.

### Step 3 (S)-2⁴-(4-acryloyl-2-methylpiperazin-1-yl)-2⁶,3⁶-difluoro-1²-isopropyl-2¹,2²-dihydro-4-aza-2(1,7)-pyrido[2,3-d]pyrimidina-1(3,4)-pyridina-3(1,2)-benzenacycloheptaphan-2²-one

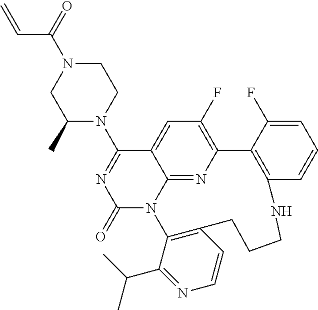 [SMILES: C=CC(=O)N1CCN(c2nc(=O)n3c4nc(c(F)cc24)-c2c(F)cccc2NCCCc2ccnc(C(C)C)c2-3)[C@@H](C)C1]

To a stirred solution of the product of Step 2 (2 g, 3.17 mmol) in DCM (20 ml) was added TFA (20 ml) at rt under Ar. The resulting mixture was stirred for 0.5 h at rt and then was concentrated to dryness. The residue was dissolved in 40 ml of DCM and the resulted solution was treated with DIPEA (4.1 g, 31.7 mmol) and acryloyl chloride (240 mg, 2.69 mmol) at 0° C. After stirring for 5 min at 0° C., the reaction was completed, the reaction was quenched with water and extracted with EtOAc. The organic layer was washed with brine, dried over Na₂SO₄, filtered and concentrated. The residue was purified by silica gel column chromatography (EtOAc:MeOH=15:1) to provide 1.38 g of the title product as a yellow solid. MS (ES+): 586.0 [M+1]⁺

¹HNMR Spectrum: (400 MHz, CDCl3) δ 8.62 (d, 1H), 7.85 (dd, 1H), 7.25-7.21 (m, 2H), 6.59-6.40 (m, 3H), 5.84 (d, 1H), 5.36 (m, 1H), 4.8-3.2 (m, 7H), 3.32 (d, 1H), 3.1-2.79 (m, 3H), 2.38-2.0 (m, 3H), 1.63-1.52 (m, 3H), 1.47-1.25 (m, 5H), 1.03-0.78 (d, 3H).

### EXAMPLE 2A and 2B

Separation of Example 2: Example 2 was separated using Daicel CHIRALPAK® IA 250*20 mm, 5 μm, at room temperature, using Hex:EtOH=60:40 as eluent with a flow rate of 15 mL/min and a UV detector at 214 nm.

Example 2A: retention time: 4.6 min, MS (ESI, m/e): 586 [M+1]⁺.

¹H NMR (400 MHz, DMSO-d₆) δ 8.47 (d, J=5.0 Hz, 1H), 8.25 (d, J=9.6 Hz, 1H), 7.39-7.22 (m, 2H), 6.87 (m, 1H), 6.69 (d, J=8.2 Hz, 1H), 6.62-6.53 (t, 1H), 6.21 (d, J=16.5 Hz, 1H), 5.78 (d, J=10.7 Hz, 1H), 4.84 (m, 2H), 4.51 (d, J=13.9 Hz, 1H), 4.33-3.98 (m, 3H), 3.57 (m, 2H), 3.31-3.11 (m, 2H), 2.80 (m, 2H), 2.42 (m, 1H), 2.20 (m, 1H), 2.04-1.84 (m, 1H), 1.43-1.20 (m, 3H), 1.08 (d, J=6.4 Hz, 3H), 0.88 (d, J=6.7 Hz, 3H).

Example 2B: retention time: 5.7 min, MS (ESI, m/e): 586 [M+1]⁺.

¹H NMR (400 MHz, DMSO-d₆) δ 8.47 (m, 2H), 7.38-7.22 (m, 2H), 6.89 (m, 1H), 6.69 (d, J=8.2 Hz, 1H), 6.63-6.51 (t, 1H), 6.23 (m, 1H), 5.78 (d, J=10.3 Hz, 1H), 5.13 (s, 1H), 4.80 (d, J=6.6 Hz, 1H), 4.31-3.70 (m, 6H), 3.23 (m, 1H), 2.93 (m, 1H), 2.80 (m, 2H), 2.60-2.44 (m, 1H), 2.18 (m, 1H), 1.90 (m, 1H), 1.29-1.18 (m, 3H), 1.12 (d, J=7.0 Hz, 3H), 0.87 (d, J=6.6 Hz, 3H).

### EXAMPLE 3

### (S)-2-(1-acryloyl-4-(2⁶,3⁶-difluoro-1²-isopropyl-2²,5-dioxo-2¹,2²-dihydro-4-aza-2(1,7)-pyrido[2,3-d]pyrimidina-1(3,4)-pyridina-3(1,2)-benzenacycloheptaphane-2⁴-yl)piperazin-2-yl)acetonitrile

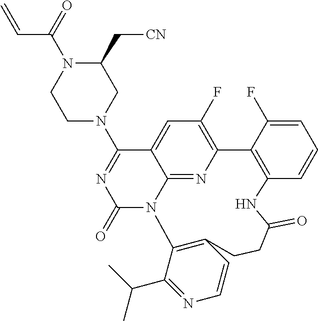 [SMILES: C=CC(=O)N1CCN(c2nc(=O)n3c4nc(c(F)cc24)-c2c(F)cccc2NC(=O)CCc2ccnc(C(C)C)c2-3)C[C@@H]1CC#N]

### Step 1 benzyl (S)-4-(1-(4-(3-(tert-butoxy)-3-oxopropyl)-2-isopropylpyridin-3-yl)-7-chloro-6-fluoro-2-oxo-1,2-dihydropyrido[2,3-d]pyrimidin-4-yl)-2-(cyanomethyl)piperazine-1-carboxylate

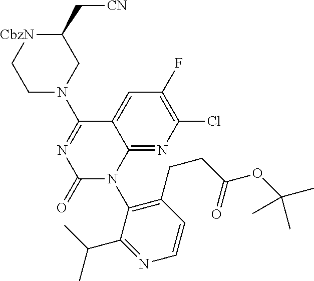 [SMILES: CC(C)c1nccc(CCC(=O)OC(C)(C)C)c1-n1c(=O)nc(N2CCN(C(=O)OCc3ccccc3)[C@@H](CC#N)C2)c2cc(F)c(Cl)nc21]

Starting from the product of Step 4 of Example 1 (150 mg, 0.325 mmol) and (S)-benzyl 2-(cyanomethyl)piperazine-1-carboxylate hydrochloride (192 mg, 0.649 mmol), the title product (256 mg) was obtained by following the conditions described in Step 8 of Example 1. MS (ESI+): 703.9[M+H]⁺

### Step 2 benzyl (2S)-4-(7-(2-amino-6-fluorophenyl)-1-(4-(3-(tert-butoxy)-3-oxopropyl)-2-isopropylpyridin-3-yl)-6-fluoro-2-oxo-1,2-dihydropyrido[2,3-d]pyrimidin-4-yl)-2-(cyanomethyl)piperazine-1-carboxylate

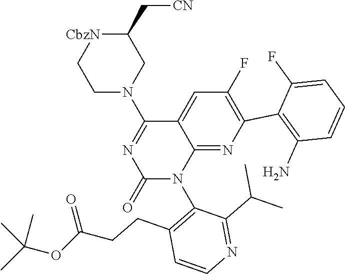 [SMILES: CC(C)c1nccc(CCC(=O)OC(C)(C)C)c1-n1c(=O)nc(N2CCN(C(=O)OCc3ccccc3)[C@@H](CC#N)C2)c2cc(F)c(-c3c(N)cccc3F)nc21]

Starting from the product of Step 1 (217 mg, 0.308 mmol), 3-fluoro-2-(4,4,5,5-tetramethyl-1,3,2-dioxaborolan-2-yl)aniline (146 mg, 0.616 mmol), the title product (158 mg) was obtained as a yellow solid by following the conditions described in Step 5 of Example 1. MS (ESI+): 779.0[M+H]⁺.

### Step 3 3-(3-(7-(2-amino-6-fluorophenyl)-4-((S)-4-((benzyloxy)carbonyl)-3-(cyanomethyl)piperazin-1-yl)-6-fluoro-2-oxopyrido[2,3-d]pyrimidin-1(2H)-yl)-2-isopropylpyridin-4-yl)propanoic acid

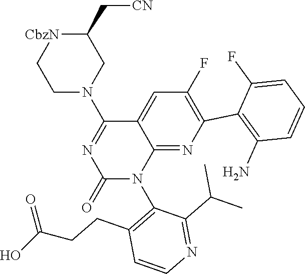 [SMILES: CC(C)c1nccc(CCC(=O)O)c1-n1c(=O)nc(N2CCN(C(=O)OCc3ccccc3)[C@@H](CC#N)C2)c2cc(F)c(-c3c(N)cccc3F)nc21]

Starting from the product of Step 2 (158 mg, 0.203 mmol), the title product (152 mg) was obtained as a yellow solid by following the conditions described in Step 6 of Example 1. MS (ESI+): 722.9[M+H]⁺.

### Step 4 benzyl (S)-2-(cyanomethyl)-4-(2⁶,3⁶-difluoro-1²-isopropyl-2²,5-dioxo-2¹,2²-dihydro-4-aza-2(1,7)-pyrido[2,3-d]pyrimidina-1(3,4)-pyridina-3(1,2)-benzenacycloheptaphane-2⁴-yl)piperazine-1-carboxylate

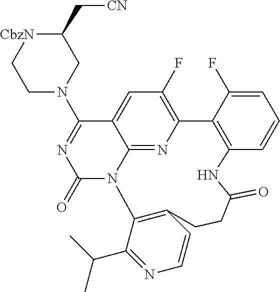 [SMILES: CC(C)c1nccc2c1-n1c(=O)nc(N3CCN(C(=O)OCc4ccccc4)[C@@H](CC#N)C3)c3cc(F)c(nc31)-c1c(F)cccc1NC(=O)CC2]

Starting from the product of Step 3 (152 mg, 0.211 mmol), the title product (96 mg) was obtained as a yellow solid by following the conditions described in Step 7 of Example 1. MS (ES+): 704.9[M+H]⁺.

### Step 5 (S)-2-(4-(2⁶,3⁶-difluoro-1²-isopropyl-2²,5-dioxo-2¹,2²-dihydro-4-aza-2(1,7)-pyrido[2,3-d]pyrimidina-1(3,4)-pyridina-3(1,2)-benzenacycloheptaphane-2⁴-yl)piperazin-2-yl)acetonitrile

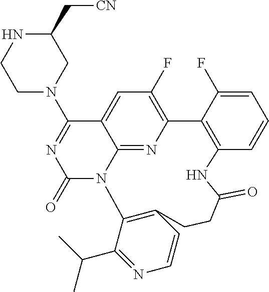 [SMILES: CC(C)c1nccc2c1-n1c(=O)nc(N3CCN[C@@H](CC#N)C3)c3cc(F)c(nc31)-c1c(F)cccc1NC(=O)CC2]

To a stirred solution of the product of Step 4 (70 mg, 0.099 mmol) in MeOH (4 ml) was added 10% Pd/C (70 mg) at rt. The resulting mixture was attired for 1.5 h at 30° C. under H₂. The reaction mixture was filtered and concentrated to give the title product as a yellow solid (48 mg). MS (ES+): 570.9[M+H]⁺.

### Step 6 (S)-2-(1-acryloyl-4-(2⁶,3⁶-difluoro-1²-isopropyl-2²,5-dioxo-2¹,2²-dihydro-4-aza-2(1,7)-pyrido[2,3-d]pyrimidina-1(3,4)-pyridina-3(1,2)-benzenacycloheptaphane-2⁴-yl)piperazin-2-yl)acetonitrile

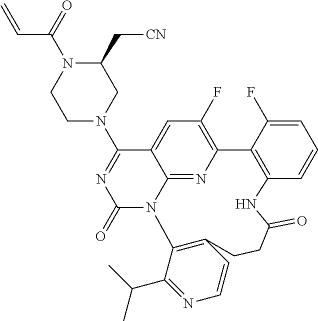 [SMILES: C=CC(=O)N1CCN(c2nc(=O)n3c4nc(c(F)cc24)-c2c(F)cccc2NC(=O)CCc2ccnc(C(C)C)c2-3)C[C@@H]1CC#N]

To a stirred solution of the product of Step 7 (48 mg, 0.084 mmol) and DIPEA (44 mg, 0.341 mmol) in DCM (1 ml) was added acryloyl chloride (8 mg, 0.084 mmol) at 5˜10° C. After stirring for 1 h at rt, the reaction mixture was quenched with water and extracted with EtOAc. The organic layer was washed with brine, dried over Na₂SO₄, filtered and concentrated. The residue was purified by Prep-TLC (EtOAc:MeOH=10:1) to give 9 mg of the title product as a yellow solid. MS (ES+): 625.0[M+H]⁺.

¹HNMR Spectrum: (400 MHz, CDCl₃) δ 8.92 (s, 1H), 8.54˜8.53 (d, J=4.4 Hz, 1H), 7.99˜7.96 (m, 1H), 7.52˜7.21 (m, 3H), 7.01 (m, 1H), 6.62 (m, 1H), 6.48 (m, 1H), 5.90˜5.88 (d, J=11.2 Hz, 1H), 5.0˜3.7 (m, 7H), 3.0˜2.8 (m, 5H), 2.61 (m, 1H), 2.33 (m, 1H), 1.32˜1.25 (m, 3H), 1.08˜1.06 (m, 3H).

### EXAMPLE 4

### (S)-2⁴-(4-acryloyl-2-methylpiperazin-1-yl)-2⁶,3⁶-difluoro-1²-isopropyl-4-methyl-2¹,2²-dihydro-4-aza-2(1,7)-pyrido[2,3-d]pyrimidina-1(3,4)-pyridina-3(1,2)-benzenacycloheptaphan-2²-one

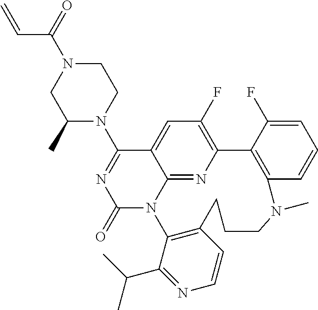 [SMILES: C=CC(=O)N1CCN(c2nc(=O)n3c4nc(c(F)cc24)-c2c(F)cccc2N(C)CCCc2ccnc(C(C)C)c2-3)[C@@H](C)C1]

To a stirred solution of Example 2 (50 mg, 0.085 mmol) and Cs₂CO₃ (111 mg, 0.342 mmol) in DMF (2 ml) was added CH₃I (42 mg, 0.299 mmol) at rt. After stirring for 5 h at 40° C., the reaction mixture was treated with water and extracted with EtOAc. The organic layer was washed with brine, dried over Na₂SO₄, filtered and concentrated. The residue was purified by Prep-TLC (EA:MeOH=15:1) to obtain 15 mg of the title product as a yellow solid. MS (ES+): 599.9 [M+1]⁺.

¹HNMR Spectrum: (400 MHz, CDCl₃) δ 8.54-8.53 (d, J=4 Hz, 1H), 7.88-7.76 (m, 1H), 7.40-7.26 (q, 1H), 7.09-7.05 (m, 2H), 6.93-6.89 (t, 1H), 6.65 (s, 1H), 6.44-6.39 (d, J=20 Hz, 1H), 5.83-5.80 (d, J=12 Hz, 1H), 5.51-4.57 (m, 2H), 4.28-3.17 (m, 4H), 2.95-2.86 (m, 3H), 2.66-2.63 (d, J=12 Hz, 1H), 2.48-2.42 (m, 5H), 1.89 (s, 1H), 1.46-1.44 (d, J=8 Hz, 2H), 1.38-1.22 (t, 5H), 1.15-0.86 (m, 3H).

### EXAMPLE 5

### (S)-2⁴-(4-acryloyl-2-methylpiperazin-1-yl)-2⁶,3⁶-difluoro-1²-isopropyl-2¹,2²-dihydro-4,6-diaza-2(1,7)-pyrido[2,3-d]pyrimidina-1(3,4)-pyridina-3(1,2)-benzenacyclooctaphane-2²,5-dione

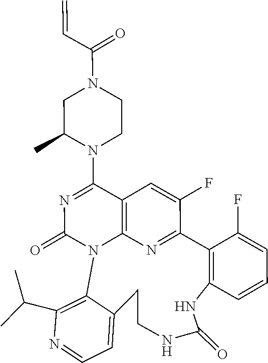 [SMILES: C=CC(=O)N1CCN(c2nc(=O)n3c4nc(c(F)cc24)-c2c(F)cccc2NC(=O)NCCc2ccnc(C(C)C)c2-3)[C@@H](C)C1]

### Step 1 2⁶,3⁶-difluoro-1²-isopropyl-2¹,2²,2³,2⁴-tetrahydro-4,6-diaza-2(1,7)-pyrido[2,3-d]pyrimidina-1(3,4)-pyridina-3(1,2)-benzenacyclooctaphane-2²,2⁴,5-trione

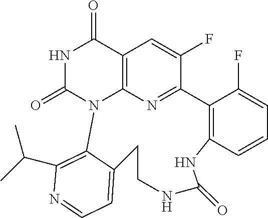 [SMILES: CC(C)c1nccc2c1-n1c(=O)[nH]c(=O)c3cc(F)c(nc31)-c1c(F)cccc1NC(=O)NCC2]

To a stirred solution of the product of Step 6 of Example 1 (408 mg, 0.85 mmol) and TEA (430.06 mg, 4.25 mmol) in toluene (15 ml) was added diphenylphosphoryl azide (701.76 mg, 2.55 mmol). After the mixture was stirred at 75° C. for 1 h, water (5 ml) and EtOAc (3 ml) were added. The mixture was stirred for 0.5 h, and the resulting solid was collected by filtration and dried to get 180 mg of the title product as a yellow solid. MS (ES+): 478.9[M+1]⁺.

### Step 2 (S)-2⁴-(4-acryloyl-2-methylpiperazin-1-yl)-2⁶,3⁶-difluoro-1²-isopropyl-2¹,2²-dihydro-4,6-diaza-2(1,7)-pyrido[2,3-d]pyrimidina-1(3,4)-pyridina-3(1,2)-benzenacyclooctaphane-2²,5-dione

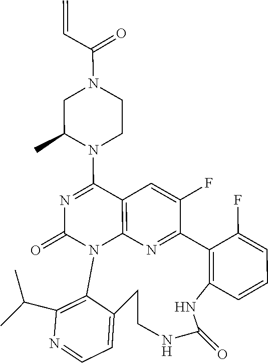 [SMILES: C=CC(=O)N1CCN(c2nc(=O)n3c4nc(c(F)cc24)-c2c(F)cccc2NC(=O)NCCc2ccnc(C(C)C)c2-3)[C@@H](C)C1]

Starting from the product of Step 1, the title product was obtained as a yellow solid by following the reaction conditions described for Step 8 and Step 9 of Example 1. MS (ES+): 614.9[M+1]⁺

¹HNMR Spectrum: (400 MHz, CDCl3) δ 11.23 (s, 1H), 8.59 (s, 1H), 7.89 (s, 1H), 7.43 (s, 1H), 7.19 (s, 1H), 7.03 (s, 1H), 6.63 (s, 1H), 6.44-6.40 (d, J=16, 1H), 5.84-5.81 (d, J=12, 1H), 4.77 (s, 2H), 4.12-3.75 (m, 2H), 3.65 (m, 2H), 3.22 (m, 2H), 2.66 (m, 1H), 1.43 (m, 5H), 1.32 (m, 3H), 1.02 (m, 3H).

### EXAMPLE 6A and 6B

2⁶,3⁶-difluoro-2⁴-((2S,5R)-4-(2-fluoroacryloyl)-2,5-dimethylpiperazin-1-yl)-1²-isopropyl-2¹,2²-dihydro-4-aza-2(1,7)-pyrido[2,3-d]pyrimidina-1(3,4)-pyridina-3(1,2)-benzenacycloheptaphan-2²-one

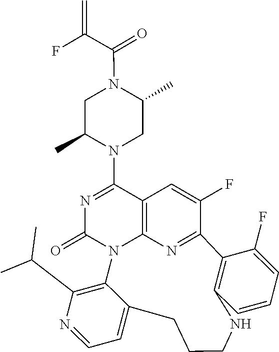 [SMILES: C=C(F)C(=O)N1C[C@H](C)N(c2nc(=O)n3c4nc(c(F)cc24)-c2c(F)cccc2NCCCc2ccnc(C(C)C)c2-3)C[C@H]1C]

### Step 1 tert-butyl (2R,5S)-4-(2⁶,3⁶-difluoro-1²-isopropyl-2²-oxo-2¹,2²-dihydro-4-aza-2(1,7)-pyrido[2,3-d]pyrimidina-1(3,4)-pyridina-3(1,2)-benzenacycloheptaphane-2⁴-yl)-2,5-dimethylpiperazine-1-carboxylate

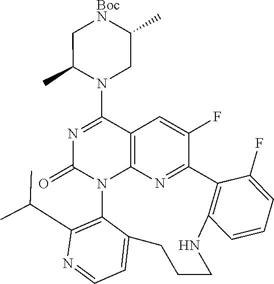 [SMILES: CC(C)c1nccc2c1-n1c(=O)nc(N3C[C@@H](C)N(C(=O)OC(C)(C)C)C[C@@H]3C)c3cc(F)c(nc31)-c1c(F)cccc1NCCC2]

To a stirred solution of the product of Step 1 of Example 2 (120 mg, 0.27 mmol) in CH₃CN (18 ml) were added DIPEA (345 mg, 2.7 mmol) and POCl₃ (414 mg, 2.7 mmol). The mixture was stirred at 80° C. for 1 h. The reaction mixture was concentrated under reduced pressure and the residue was dissolved in DMF (1 ml). The resulted solution was treated with DIEA (172 mg, 1.4 mmol) and (2R,5S)-tert-butyl 2,5-dimethylpiperazine-1-carboxylate (64 mg, 0.3 mmol) at 0° C. After stirring for 10 min at at 0° C., the reaction was quenched with water and extracted with EtOAc. The organic layer was washed with brine, dried over Na₂SO₄, filtered and concentrated to get 182 mg of the title compound as a yellow solid. MS (ES+): 645.9[M+1]⁺

### Step 2 24-((2S,5R)-2,5-dimethylpiperazin-1-yl)-2⁶,3⁶-difluoro-1²-isopropyl-2¹,2²-dihydro-4-aza-2(1,7)-pyrido[2,3-d]pyrimidina-1(3,4)-pyridina-3(1,2)-benzenacycloheptaphan-2²-one

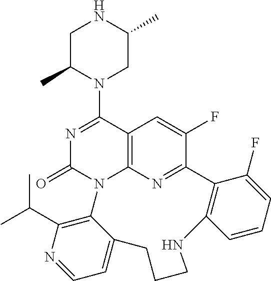 [SMILES: CC(C)c1nccc2c1-n1c(=O)nc(N3C[C@@H](C)NC[C@@H]3C)c3cc(F)c(nc31)-c1c(F)cccc1NCCC2]

To a stirred solution of the product of the Step 1(182 mg, 0.28 mmol) in DCM (1 ml) was added TFA (1 ml). After stirring at rt for 1 h, the reaction mixture was concentrated, basified to pH 7-8 with NaHCO₃(aq.) and extracted with EA. The organic layer was washed with brine, dried over Na₂SO₄, filtered and concentrated to get 141 mg of the target compound as a yellow solid. MS (ES+): 545.9[M+1]⁺.

### Step 3 2⁶,3⁶-difluoro-2⁴-((2S,5R)-4-(2-fluoroacryloyl)-2,5-dimethylpiperazin-1-yl)-1²-isopropyl-2¹,2²-dihydro-4-aza-2(1,7)-pyrido[2,3-d]pyrimidina-1(3,4)-pyridina-3(1,2)-benzenacycloheptaphan-2²-one

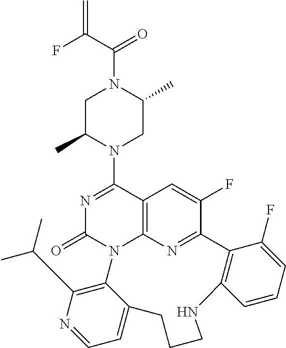 [SMILES: C=C(F)C(=O)N1C[C@H](C)N(c2nc(=O)n3c4nc(c(F)cc24)-c2c(F)cccc2NCCCc2ccnc(C(C)C)c2-3)C[C@H]1C]

To a solution of the product of the step 2 (70 mg, 0.13 mmol) in DCM (3 ml) were added 2-fluoroprop-2-enoic acid (9.37 mg, 0.1 mmol), DIPEA (84 mg, 0.65 mmol) and T3P (124 mg, 0.39 mmol). After stirring at rt for 10 min, the reaction was quenched with saturated aqueous NaHCO₃(15 ml) and extracted with DCM. The organic layer was washed with brine, dried over Na₂SO₄, filtered and concentrated. The residue was purified by Prep-TLC (MeOH/EA=5%) to give rise to 15.5 mg of Example 6A (fast eluting) as a yellow solid, MS (ES+): 617.9[M+1]⁺, and 15.2 mg of Example 6B (slow eluting) as a yellow solid, MS (ES+): 617.9[M+1]⁺.

¹HNMR Spectrum (Example 6A): (400 MHz, CDCl₃) δ 8.47-8.46 (d, J=4 Hz, 2H), 7.34-7.26 (m, 2H), 6.72-6.54 (m, 2H), 5.37-5.32 (m, 2H), 4.87-4.88 (m, 2H), 4.75-4.25 (m, 1H), 4.12-4.01 (m, 2H), 3.78-3.63 (m, 1H), 3.17 (s, 1H), 2.84 (m, 1H), 2.76-2.73 (m, 1H), 2.45 (m, 1H), 2.12-1.93 (m, 2H), 1.27-1.09 (m, 10H), 0.84-0.82 (d, J=8 Hz, 3H).

¹HNMR Spectrum (Example 6B): (400 MHz, CDCl₃) δ 8.47-8.45 (d, J=4 Hz, 1H), 8.11 (m, 1H), 7.33-7.24 (m, 2H), 6.71-6.55 (m, 2H), 5.37-5.32 (d, J=20 Hz, 2H), 4.88-4.61 (m, 3H), 4.52-4.11 (m, 1H), 3.88-3.46 (m, 2H), 3.25 (m, 1H), 2.89-2.73 (m, 2H), 2.57 (m, 1H), 2.18-1.91 (m, 2H), 1.50-1.48 (d, J=8 Hz, 2H), 1.38-1.25 (m, 4H), 1.08-1.06 (d, J=8 Hz, 3H), 0.93-0.91 (d, J=8 Hz, 3H).

### EXAMPLE 7A and 7B

### 2⁴-((2S,5R)-4-acryloyl-2,5-dimethylpiperazin-1-yl)-2⁶,3⁶-difluoro-1²-isopropyl-2¹,2²-dihydro-4-aza-2(1,7)-pyrido[2,3-d]pyrimidina-1(3,4)-pyridina-3(1,2)-benzenacycloheptaphan-2²-one

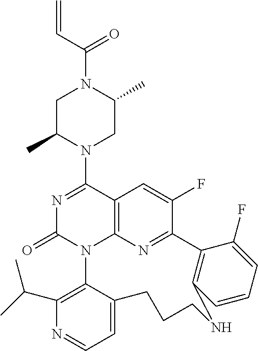 [SMILES: C=CC(=O)N1C[C@H](C)N(c2nc(=O)n3c4nc(c(F)cc24)-c2c(F)cccc2NCCCc2ccnc(C(C)C)c2-3)C[C@H]1C]

To a stirred solution of the product of Step 2 of Example 6A and 6B (0.07 g, 0.128 mmol), acrylic acid (0.007 g, 0.103 mmol) and DIPEA (0.083 g, 0.642 mmol) in DCM (3 ml) was added T3P (0.123 g, 0.385 mmol). After stirring at rt for 0.5 h under Ar, the reaction was quenched with NaHCO₃(aq.) and extracted with DCM, the organic layer was washed with brine, dried over Na₂SO₄, filtered and concentrated. The residue was purified by Prep-TLC (MeOH:EA=7.5%) to give 9.8 mg of Example 7A (fast eluting) as a yellow solid, MS (ES+): 599.9[M+1]⁺, and 9.8 mg of Example 7B (slow eluting) as a yellow solid, MS (ES+): 599.9[M+1]⁺.

¹HNMR Spectrum (Example 7A): (400 MHz, CDCl₃) δ 8.49 (m, 2H), 7.34-7.26 (m, 1H), 6.86-6.70 (m, 2H), 6.59-6.55 (m, 1H), 6.22-6.17 (m, 1H), 5.77-5.74 (m, 1H), 4.97-4.87 (m, 2H), 4.75-4.25 (m, 1H), 4.07-3.86 (m, 4H), 2.84-2.66 (m, 3H), 2.13-1.78 (m, 3H), 1.36-1.24 (d, 2H), 1.18 (d, 1H), 1.17-1.06 (m, 3H), 0.88-0.78 (d, 4H).

¹HNMR Spectrum (Example 7B): (400 MHz, CDCl₃) δ 8.46-8.45 (d, J=4 Hz, 1H), 8.12-8.06 (m, 1H), 7.33-7.26 (m, 2H), 6.87-6.55 (m, 3H), 6.21-6.16 (m, 1H), 5.78-5.75 (m, 1H), 4.89-4.47 (m, 4H), 4.27-3.56 (m, 3H), 2.84-2.78 (m, 2H), 2.46-1.87 (m, 2H), 1.48-1.45 (m, 3H), 1.32-1.28 (m, 3H), 1.10-1.06 (m, 3H), 0.92-0.78 (m, 3H).

### EXAMPLE 8

### (S)-2⁴-(4-acryloyl-2-methylpiperazin-1-yl)-2⁶,3⁶-difluoro-1²-isopropyl-2¹,2²-dihydro-8-thia-4-aza-2(1,7)-pyrido[2,3-d]pyrimidina-1(3,4)-pyridina-3(1,2)-benzenacyclooctaphan-2²-one

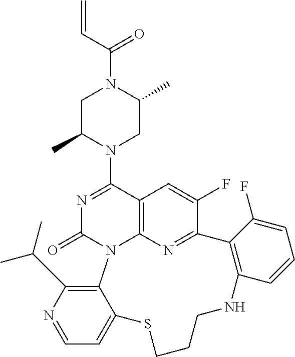 [SMILES: C=CC(=O)N1C[C@H](C)N(c2nc(=O)n3c4nc(c(F)cc24)-c2c(F)cccc2NCCCSc2ccnc(C(C)C)c2-3)C[C@H]1C]

### Step 1 methyl 3-(3-amino-2-isopropylpyridin-4-ylthio)propanoate

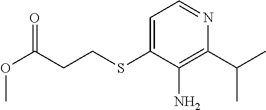 [SMILES: COC(=O)CCSc1ccnc(C(C)C)c1N]

The mixture of 4-iodo-2-isopropylpyridin-3-amine (625 mg, 2.385 mmol), methyl 3-sulfanylpropanoate (716 mg, 5.964 mmol), DIPEA (1.231 g, 9.542 mmol), Pd₂(dba)₃ (437 mg, 0.477 mmol) and Xantphos (552 mg, 0.954 mmol) in 1,4-Dioxane (15 ml) was stirred for 2 h at 80° C. under Ar. The reaction mixture was treated with water and extracted with EtOAc. The organic layer was washed with brine, dried over Na₂SO₄, filtered and concentrated. The residue was purified by silica gel column chromatography (EA:PE=1:1) to give 582 mg of the title product as a yellow solid. MS (ES+): 255 [M+1]⁺.

### Step 2 methyl 3-(3-(3-(2,6-dichloro-5-fluoronicotinoyl)ureido)-2-isopropylpyridin-4-ylthio)propanoate

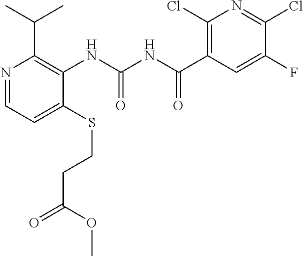 [SMILES: COC(=O)CCSc1ccnc(C(C)C)c1NC(=O)NC(=O)c1cc(F)c(Cl)nc1Cl]

Starting with 2,6-dichloro-5-fluoronicotinamide and the product of Step 1, the title product was obtained as a yellow solid by following the procedure described in Step 3 of Example 1. MS (ES+): 489 [M+1]⁺

### Step 3 methyl 3-(3-(7-chloro-6-fluoro-4-hydroxy-2-oxopyrido[2,3-d]pyrimidin-1(2H)-yl)-2-isopropylpyridin-4-ylthio)propanoate

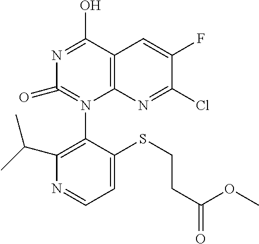

The mixture of the product of Step 2 (900 mg, 1.844 mmol) and K₂CO₃ (509 mg, 3.689 mmol) in DMF (15 ml) was stirred for 18 h at rt. Water was added and the mixture was extracted with EtOAc. The organic layer was washed with brine, dried over Na₂SO₄, filtered and concentrated to give 804 mg of the title product as a yellow solid. MS (ES+): 453 [M+1]⁺.

### Step 4 methyl 3-(3-(7-(2-amino-6-fluorophenyl)-6-fluoro-2,4-dioxo-3,4-dihydropyrido[2,3-d]pyrimidin-1(2H)-yl)-2-isopropylpyridin-4-ylthio)propanoate

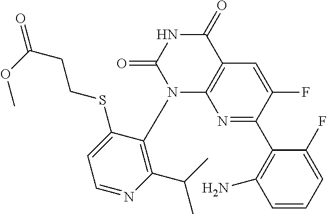 [SMILES: COC(=O)CCSc1ccnc(C(C)C)c1-n1c(=O)[nH]c(=O)c2cc(F)c(-c3c(N)cccc3F)nc21]

Starting with the product of Step 3 and 3-fluoro-2-(4,4,5,5-tetramethyl-1,3,2-dioxaborolan-2-yl)aniline, the title product was obtained as a yellow solid by following the procedure described in Step 5 of Example 1. MS (ES+): 527.8 [M+1]⁺

### Step 5 3-(3-(7-(2-amino-6-fluorophenyl)-6-fluoro-2,4-dioxo-3,4-dihydropyrido[2,3-d]pyrimidin-1(2H)-yl)-2-isopropylpyridin-4-ylthio)propanoic acid

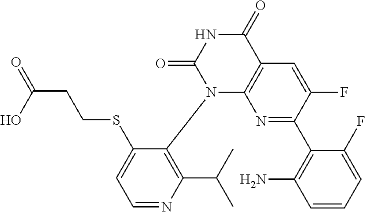 [SMILES: CC(C)c1nccc(SCCC(=O)O)c1-n1c(=O)[nH]c(=O)c2cc(F)c(-c3c(N)cccc3F)nc21]

A mixture of the product of Step 4 (300 mg, 28.8 mmol) in 6 N HCl/THF (30 ml/5 ml) was stirred overnight at rt. The pH of reaction mixture was adjusted to 5-6 with NaHCO₃(aq.) and extracted with EtOAc. The organic layer was washed with brine, dried over Na₂SO₄, filtered and concentrated to give 360 mg of the title product as a brown solid. MS (ES+): 513.8 [M+1]⁺.

### Step 6 2⁶,3⁶-difluoro-12-isopropyl-2¹,2²,2³,2⁴-tetrahydro-8-thia-4-aza-2(1,7)-pyrido[2,3-d]pyrimidina-1(3,4)-pyridina-3(1,2)-benzenacyclooctaphane-2²,2⁴,5-trione

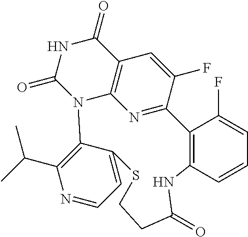 [SMILES: CC(C)c1nccc2c1-n1c(=O)[nH]c(=O)c3cc(F)c(nc31)-c1c(F)cccc1NC(=O)CCS2]

To a stirred solution of the product of Step 5 (360 mg, 0.70 mmol) in DCE (12 ml) was added T3P in EtOAc (50% wt, 2.2 g, 3.5 mmol) at rt under Ar. The mixture was stirred overnight at 55° C. The pH of reaction mixture was adjusted to 5-6 with NaHCO₃(aq.) and extracted with EtOAc. The organic layer was washed with brine, dried over Na₂SO₄, filtered and concentrated. The residue was purified by Prep-TLC (EA:MeOH=15:1) to give 130 mg of the title product as a yellow solid. MS (ES+): 495.8 [M+1]⁺

### Step 7 2⁶,3⁶-difluoro-1²-isopropyl-2¹,2²,2³,2⁴-tetrahydro-8-thia-4-aza-2(1,7)-pyrido[2,3-d]pyrimidina-1(3,4)-pyridina-3(1,2)-benzenacyclooctaphane-2²,2⁴-dione

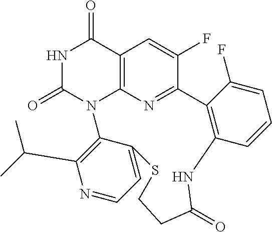 [SMILES: CC(C)c1nccc2c1-n1c(=O)[nH]c(=O)c3cc(F)c(nc31)-c1c(F)cccc1NC(=O)CCS2]

To a stirred the product of Step 6 (130 mg, 0.26 mmol) in THF (7 ml) was added borane-tetrahydrofuran complex in THF (1.0 M, 1.3 ml, 1.3 mmol) at 0° C. under Ar. After stirring for 1 h at rt, additional borane-tetrahydrofuran complex in THF (1.0 M, 0.8 ml, 0.8 mmol) was added at 0° C. under Ar. The mixture was stirred for another 1 h at rt, and then quenched with water and extracted with EtOAc. The organic layer was washed with brine, dried over Na₂SO₄, filtered and concentrated. The residue was purified by Prep-TLC (EA:MeOH=15:1) to give 69 mg of the title product as a white solid. MS (ES+): 481.9 [M+1]⁺.

### Step 8 tert-butyl (S)-4-(2⁶,3⁶-difluoro-1²-isopropyl-2²-oxo-2¹,2²-dihydro-8-thia-4-aza-2(1,7)-pyrido[2,3-d]pyrimidina-1(3,4)-pyridina-3(1,2)-benzenacyclooctaphane-2⁴-yl)-3-methylpiperazine-1-carboxylate

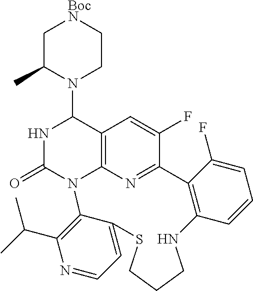 [SMILES: CC(C)c1nccc2c1N1C(=O)NC(N3CCN(C(=O)OC(C)(C)C)C[C@@H]3C)c3cc(F)c(nc31)-c1c(F)cccc1NCCCS2]

Starting with the product of Step 7 and 3 tert-butyl (S)-3-methylpiperazine-1-carboxylate, the title product was obtained as a yellow solid by following the procedure described in Step 2 of Example 2. MS (ES+): 663.8 [M+1]⁺.

### Step 9 (S)-2⁴-(4-acryloyl-2-methylpiperazin-1-yl)-2⁶,3⁶-difluoro-1²-isopropyl-2¹,2²-dihydro-8-thia-4-aza-2(1,7)-pyrido[2,3-d]pyrimidina-1(3,4)-pyridina-3(1,2)-benzenacyclooctaphan-2²-one

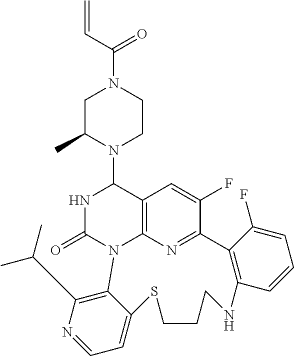 [SMILES: C=CC(=O)N1CCN(C2NC(=O)N3c4nc(c(F)cc42)-c2c(F)cccc2NCCCSc2ccnc(C(C)C)c23)[C@@H](C)C1]

Starting with the product of Step 8, the title product was obtained as a yellow solid by following the procedure described in Step 3 of Example 2. MS (ES+): 617.8 [M+1]⁺.

¹HNMR Spectrum: (400 MHz, CDCl₃) δ 8.53-8.52 (d, J=4 Hz, 1H), 7.83 (m, 1H), 7.26-7.21 (m, 1H), 7.15-7.13 (d, J=8 Hz, 1H), 6.64 (s, 2H), 6.42-6.38 (m, 3H), 5.82-5.80 (d, J=8 Hz, 1H), 4.98-4.41 (m, 2H), 4.38-3.61 (m, 3H), 3.47-3.32 (m, 3H), 3.14-3.02 (m, 3H), 2.78 (m, 1H), 2.22 (m, 1H), 2.07-1.68 (m, 3H), 1.64-1.55 (m, 2H), 1.39-1.37 (d, J=8 Hz, 2H), 1.01-0.99 (d, J=8 Hz, 3H).

### EXAMPLE 9

### (S)-2⁴-(4-acryloyl-2-methylpiperazin-1-yl)-2⁶,3⁶-difluoro-1²-isopropyl-2¹,2²-dihydro-7-thia-4-aza-2(1,7)-pyrido[2,3-d]pyrimidina-1(3,4)-pyridina-3(1,2)-benzenacycloheptaphan-2²-one

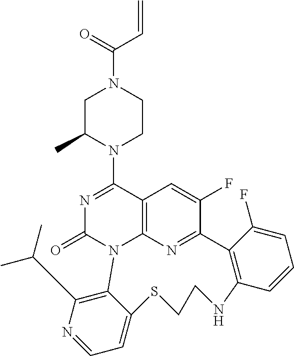 [SMILES: C=CC(=O)N1CCN(c2nc(=O)n3c4nc(c(F)cc24)-c2c(F)cccc2NCCSc2ccnc(C(C)C)c2-3)[C@@H](C)C1]

### Step 1 2-isopropyl-4-((4-methoxybenzyl)thio)pyridin-3-amine

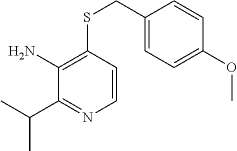 [SMILES: COc1ccc(CSc2ccnc(C(C)C)c2N)cc1]

A mixture of 4-iodo-2-isopropylpyridin-3-amine (320 mg, 1.2 mmol), (4-methoxyphenyl)methanethiol (376 mg, 2.4 mmol), DIPEA (630 mg, 4.9 mmol), Xantphos (283 mg, 0.49 mmol) and Pd₂(dba)₃ (224 mg, 0.24 mmol) in dioxane (10 ml) was stirred at 90° C. for 2 h. The reaction was quenched with water at rt and extracted with EA. The organic layer was washed with brine, dried over Na₂SO₄, filtered and concentrated. The residue was purified by silica gel column chromatography (PE:EA=5:1) to give 382 mg of the title product as a yellow solid. MS (ES+): 288.8 [M+1]⁺.

### Step 2 7-chloro-6-fluoro-4-hydroxy-1-(2-isopropyl-4-((4-methoxybenzyl)thio)pyridin-3-yl)pyrido[2,3-d]pyrimidin-2(1H)-one

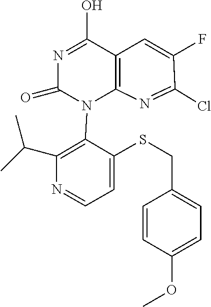 [SMILES: COc1ccc(CSc2ccnc(C(C)C)c2-n2c(=O)nc(O)c3cc(F)c(Cl)nc32)cc1]

Starting from the product of Step 1 and 2,6-dichloro-5-fluoronicotinamide, the title product was obtained as a white solid by following the procedures described in Step 3 and Step 4 of Example 1. MS (ES+): 486.9[M+1]⁺.

### Step 3 7-chloro-6-fluoro-4-hydroxy-1-(2-isopropyl-4-mercaptopyridin-3-yl)pyrido[2,3-d]pyrimidin-2(1H)-one

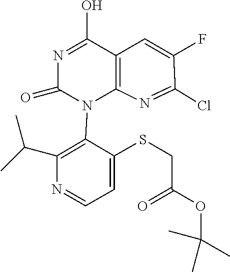 [SMILES: CC(C)c1nccc(SCC(=O)OC(C)(C)C)c1-n1c(=O)nc(O)c2cc(F)c(Cl)nc21]

To a stirred solution of the product of Step 2 (538 mg, 1.1 mmol) in TFA (12 ml) was added trifluoromethanesulfonic anhydride (620.7 mg, 2.2 mmol, dissolved in 1 ml of TFA) at rt under Ar. The mixture was stirred for 2.5 h at 80° C. and was concentrated to dryness. The residue was dissolved in DMF (12 ml) and the resulted solution was treated with TEA (2.0 g, 19.8 mmol) and tert-Butyl bromoacetate (171.6 mg, 0.88 mmol, dissolved in 1 ml of DMF). After stirring for 30 min, water was added and the mixture was extracted with EtOAc. The organic layer was washed with brine, dried over Na₂SO₄, filtered and concentrated. The residue was purified by silica gel column chromatography (PE:EA=1:1) to obtain 144 mg of the title product as a yellow solid. MS (ES+): 480.9[M+1]⁺.

### Step 4 2⁶,3⁶-difluoro-2⁴-hydroxy-1²-isopropyl-2¹,2²-dihydro-7-thia-4-aza-2(1,7)-pyrido[2,3-d]pyrimidina-1(3,4)-pyridina-3(1,2)-benzenacycloheptaphane-2²,5-dione

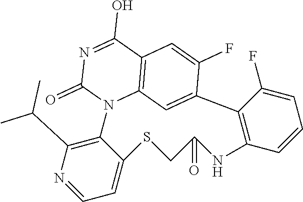 [SMILES: CC(C)c1nccc2c1-n1c(=O)nc(O)c3cc(F)c(cc31)-c1c(F)cccc1NC(=O)CS2]

Starting with the product of Step 3 and 3-fluoro-2-(tetramethyl-1,3,2-dioxaborolan-2-yl)aniline, the title product was obtained as a white solid by following the procedures described in Step 5 and Step 6 and Step 7 of Example 1. MS (ES+): 481.8[M+1]⁺.

### Step 5 (S)-2⁴-(4-acryloyl-2-methylpiperazin-1-yl)-2⁶,3⁶-difluoro-1²-isopropyl-2¹,2²-dihydro-7-thia-4-aza-2(1,7)-pyrido[2,3-d]pyrimidina-1(3,4)-pyridina-3(1,2)-benzenacycloheptaphan-2²-one

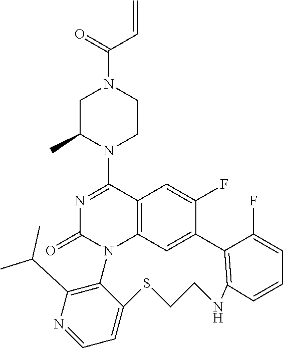 [SMILES: C=CC(=O)N1CCN(c2nc(=O)n3c4cc(c(F)cc24)-c2c(F)cccc2NCCSc2ccnc(C(C)C)c2-3)[C@@H](C)C1]

Starting from the product of Step 4, the title product was obtained as a yellow solid by following the procedures described in Step 7, Step 8 and Step 9 of Example 8. MS (ES+): 604.1 [M+1]⁺.

¹HNMR Spectrum: (400 MHz, DMSO-d₆) δ 8.54-8.50 (m, 1H), 8.48-8.47 (d, J=4 Hz, 1H), 8.34-8.32 (m, 1H), 7.62-7.60 (m, 1H), 7.46-7.42 (m, 1H), 7.35-7.25 (m, 2H), 6.88-6.84 (m, 1H), 6.70-6.68 (m, 1H), 6.65-6.60 (t, 1H), 6.23-6.19 (d, J=16 Hz, 1H), 5.79-5.76 (m, 1H), 4.24 (m, 1H), 4.31-4.20 (m, 1H), 3.99-3.95 (m, 2H), 3.63-3.60 (m, 2H), 3.01-2.93 (m, 3H), 2.91-2.80 (m, 1H), 2.57-2.54 (m, 1H), 1.43 (m, 1H), 1.24 (m, 3H), 1.16-1.13 (m, 2H), 1.12-1.11 (m, 3H), 0.91-0.89 (d, J=8 Hz, 3H).

### EXAMPLE 10

### (S)-2⁴-(4-acryloyl-2-methylpiperazin-1-yl)-3⁶-chloro-2⁶-fluoro-1²-isopropyl-2¹,2²-dihydro-4-aza-2(1,7)-pyrido[2,3-d]pyrimidina-1(3,4)-pyridina-3(1,2)-benzenacycloheptaphan-2²-one

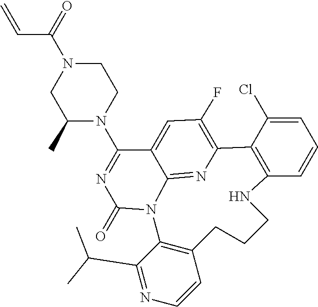 [SMILES: C=CC(=O)N1CCN(c2nc(=O)n3c4nc(c(F)cc24)-c2c(Cl)cccc2NCCCc2ccnc(C(C)C)c2-3)[C@@H](C)C1]

### Step 13-chloro-2-(4,4,5,5-tetramethyl-1,3,2-dioxaborolan-2-yl)aniline

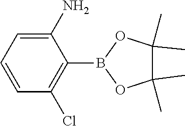 [SMILES: CC1(C)OB(c2c(N)cccc2Cl)OC1(C)C]

A mixture of 2-bromo-3-chloroaniline (10 g, 48.43 mmol), 4,4,5,5-tetramethyl-2-(tetramethyl-1,3,2-dioxaborolan-2-yl)-1,3,2-dioxaborolane (15.99 g, 62.96 mmol), potassium acetate (14.26 g, 145.29 mmol) and [1,1′-Bis(diphenylphosphino)ferrocene]dichloropalladium(II) (3.54 g, 4.84 mmol) in 1,4-dioxane (150 ml) was stirred for 14 h at 105° C. under Ar. The mixture was cooled to rt and filtered through a layer of celite, the filter cake was washed with EtOAc. The filtrate was washed with brine, dried over Na₂SO₄, filtered and concentrated to get 17.51 g of crude product as black oil.

### Step 2 tert-butyl 3-(3-(7-(2-amino-6-chlorophenyl)-6-fluoro-2,4-dioxo-3,4-dihydropyrido[2,3-d]pyrimidin-1(2H)-yl)-2-isopropylpyridin-4-yl)propanoate

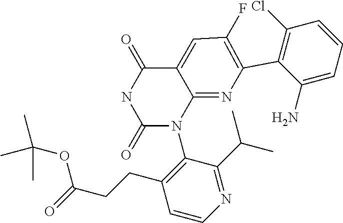 [SMILES: CC(C)c1nccc(CCC(=O)OC(C)(C)C)c1-n1c(=O)[nH]c(=O)c2cc(F)c(-c3c(N)cccc3Cl)nc21]

To a stirred solution of the product of Step 4 of Example 1 and potassium acetate (0.64 g, 6.48 mmol) in 1,4-dioxane (40 ml) were added [1,1′-Bis(diphenylphosphino)ferrocene]dichloropalladium(II) complex with dichloromethane (0.18 g, 0.22 mmol) and H₂O (1 ml). The resulting mixture was stirred for 2 h at 80° C. under Ar. Water was added and the reaction mixture was extracted with EtOAc. The organic layer was washed with brine, dried over Na₂SO₄, filtered and concentrated. The residue was purified by silica gel column chromatography (EA:PE=1:1) to give 2.39 g of the title product as yellow oil. MS (ES+): 553.8 [M+1]⁺.

### Step 3 3-(3-(7-(2-amino-6-chlorophenyl)-6-fluoro-2,4-dioxo-3,4-dihydropyrido[2,3-d]pyrimidin-1(2H)-yl)-2-isopropylpyridin-4-yl)propanoic acid

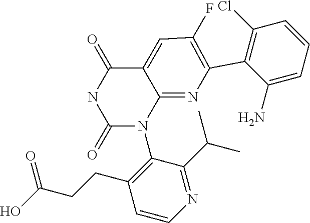 [SMILES: CC(C)c1nccc(CCC(=O)O)c1-n1c(=O)[nH]c(=O)c2cc(F)c(-c3c(N)cccc3Cl)nc21]

A mixture of the product of Step 2 (600 mg, 1.085 mmol) in TFA (6 ml) was stirred for 0.5 h at rt. The reaction mixture was concentrated to dryness to give 859 mg of the title product as brown oil. MS (ES+): 497.8[M+1]⁺.

### Step 4 3⁶-chloro-2⁶-fluoro-1²-isopropyl-2¹,2²,2³,2⁴-tetrahydro-4-aza-2(1,7)-pyrido[2,3-d]pyrimidina-1(3,4)-pyridina-3(1,2)-benzenacycloheptaphane-2²,2⁴,5-trione

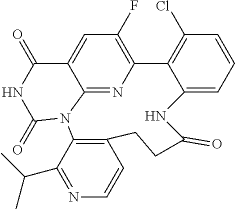 [SMILES: CC(C)c1nccc2c1-n1c(=O)[nH]c(=O)c3cc(F)c(nc31)-c1c(Cl)cccc1NC(=O)CC2]

To a stirred solution of the product of Step 3 (590 mg, 1.18 mmol) in DCE (30 ml) was added T3P (3.78 g, 5.93 mmol) at rt under Ar. After stirring for 1 h at 50° C., the reaction was quenched with sat. NaHCO₃(aq.) and extracted with EtOAc. The organic layer was washed with brine, dried over Na₂SO₄, filtered and concentrated. The residue was purified by silica gel column chromatography (MeOH/EA=2%) to give 165 mg of the title product as a yellow solid. MS (ES+): 479.8 [M+1]⁺.

### Step 5 3⁶-chloro-2⁶-fluoro-1²-isopropyl-2¹,2²,2³,2⁴-tetrahydro-4-aza-2(1,7)-pyrido[2,3-d]pyrimidina-1(3,4)-pyridina-3(1,2)-benzenacycloheptaphane-2²,2⁴-dione

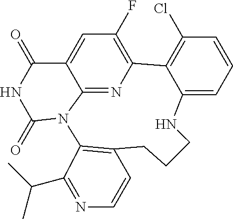 [SMILES: CC(C)c1nccc2c1-n1c(=O)[nH]c(=O)c3cc(F)c(nc31)-c1c(Cl)cccc1NCCC2]

Starting from the product of Step 4, the title compound was obtained as a yellow solid by following the reaction conditions described in Step 1 of Example 2. MS (ES+): 465.8 [M+1]⁺.

### Step 6 (S)-2⁴-(4-acryloyl-2-methylpiperazin-1-yl)-3⁶-chloro-2⁶-fluoro-1²-isopropyl-2¹,2²-dihydro-4-aza-2(1,7)-pyrido[2,3-d]pyrimidina-1(3,4)-pyridina-3(1,2)-benzenacycloheptaphan-2²-one

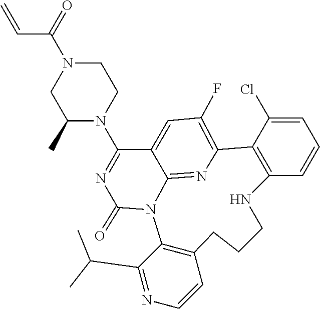 [SMILES: C=CC(=O)N1CCN(c2nc(=O)n3c4nc(c(F)cc24)-c2c(Cl)cccc2NCCCc2ccnc(C(C)C)c2-3)[C@@H](C)C1]

Starting from the product of Step 5, the title product was obtained as a yellow solid by following the procedures described in Step 2, and Step 3 of Example 2. MS (ES+): 601.8 [M+1]⁺.

¹HNMR Spectrum: (400 MHz, CDCl₃) δ 8.58-8.57 (d, J=4 Hz, 1H), 7.93-7.87 (m, 1H), 7.26-7.15 (m, 2H), 6.88-6.86 (d, J=8 Hz, 1H), 6.73-6.71 (d, J=8 Hz, 1H), 6.60 (s, 1H), 6.44-6.40 (m, 1H), 5.84-5.81 (d, J=12 Hz, 1H), 5.46-5.30 (m, 1H), 4.78-4.23 (m, 4H), 3.85-3.39 (m, 3H), 2.91-2.71 (m, 3H), 2.30-1.94 (m, 2H), 1.68-1.58 (m, 3H), 1.39-1.37 (d, J=8 Hz, 2H), 1.29-1.26 (m, 3H), 1.06-1.04 (d, J=8 Hz, 3H).

### EXAMPLE 11

### (S)-2⁶,3⁶-difluoro-2⁴-(4-(2-fluoroacryloyl)-2-methylpiperazin-1-yl)-1²-isopropyl-2¹,2²-dihydro-4-aza-2(1,7)-pyrido[2,3-d]pyrimidina-1(3,4)-pyridina-3(1,2)-benzenacycloheptaphan-2²-one

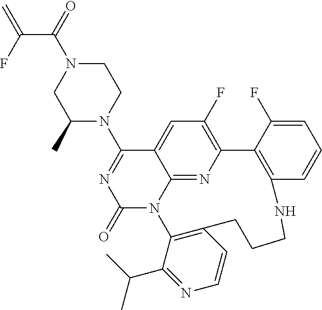 [SMILES: C=C(F)C(=O)N1CCN(c2nc(=O)n3c4nc(c(F)cc24)-c2c(F)cccc2NCCCc2ccnc(C(C)C)c2-3)[C@@H](C)C1]

To a stirred solution of the product of Step 2 od Example 2 and 2-fluoroacrylic acid, the title compound was obtained as a yellow solid by following the reaction conditions described in Step 2 and Step 3 of Example 6A and 6B. MS (ES+): 603.9[M+1]⁺.

¹HNMR Spectrum: (400 MHz, DMSO-d₆) δ 8.47-8.45 (m, 2H), 7.73-7.25 (m, 2H), 6.70-6.55 (m, 2H), 5.41-5.36 (m, 3H), 4.8-4.79 (m, 1H), 4.50-3.45 (m, 6H), 2.80-2.78 (m, 2H), 2.51-2.44 (m, 2H), 2.23-2.22 (m, 1H), 1.98-1.88 (m, 1H), 1.40-0.08 (m, 11H).

### EXAMPLE 12

### (S)-4-acetyl-2⁴-(4-acryloyl-2-methylpiperazin-1-yl)-2⁶,3⁶-difluoro-1²-isopropyl-2¹,2²-dihydro-4-aza-2(1,7)-pyrido[2,3-d]pyrimidina-1(3,4)-pyridina-3(1,2)-benzenacycloheptaphan-2²-one

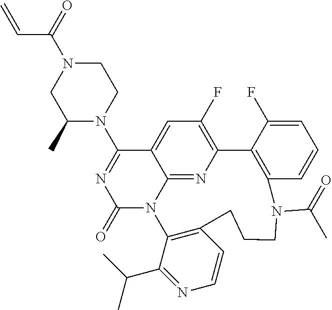 [SMILES: C=CC(=O)N1CCN(c2nc(=O)n3c4nc(c(F)cc24)-c2c(F)cccc2N(C(C)=O)CCCc2ccnc(C(C)C)c2-3)[C@@H](C)C1]

To a stirred solution of Example 2 (50 mg, 0.085 mmol) and DIPEA (58 mg, 0.45 mmol) in DCM (2 ml) was added acetyl chloride (17.6 mg, 0.22 mmol) at rt. After stirring for 2 h at 40° C., the reaction was quenched with sat.NaHCO₃(aq.) and extracted with EtOAc. The organic layer was washed with brine, dried over Na₂SO₄, filtered and concentrated. The residue was purified by Prep-TLC (EA:MeOH=5:1) to obtain 21 mg of the title product as a yellow solid. MS (ES+): 627.9 [M+1]⁺.

¹HNMR Spectrum: (400 MHz, CDCl3) δ 8.49-8.48 (d, J=4 Hz, 1H), 7.88-7.83 (t, 1H), 7.47 (q, 1H), 7.18 (t, 1H), 6.99 (t, 2H), 6.62 (s, 1H), 6.43 (d, 1H), 5.84-5.81 (d, J=12 Hz, 1H), 5.13-4.37 (m, 2H), 4.26-3.28 (m, 5H), 3.26-2.66 (m, 3H), 2.65-2.36 (m, 2H), 2.04 (s, 1H), 1.43-1.41 (d, J=8 Hz, 2H), 1.31-1.18 (m, 8H), 1.01-0.99 (d, J=8 Hz, 3H).

### EXAMPLE 13

### (S)-2⁴-(4-acryloyl-2-methylpiperazin-1-yl)-2⁶,3⁶-difluoro-1²-isopropyl-N,N-dimethyl-2²-oxo-2¹,2²-dihydro-4-aza-2(1,7)-pyrido[2,3-d]pyrimidina-1(3,4)-pyridina-3(1,2)-benzenacycloheptaphane-4-carboxamide

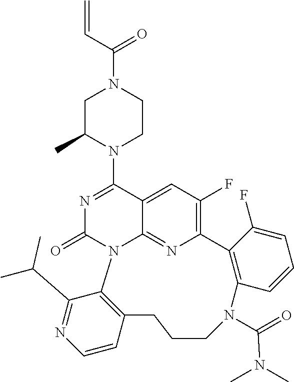 [SMILES: C=CC(=O)N1CCN(c2nc(=O)n3c4nc(c(F)cc24)-c2c(F)cccc2N(C(=O)N(C)C)CCCc2ccnc(C(C)C)c2-3)[C@@H](C)C1]

To a stirred solution of Example 2 (50 mg, 0.085 mmol) in DCE (3 ml) were added dimethylcarbamic chloride (18 mg, 0.171 mmol), DIEA (33 mg, 0.256 mmol) and DMAP (10 mg, 0.085 mmol). The resulting mixture was stirred overnight at 80° C. and then quenched with water, and extracted with EtOAc. The organic layer was washed with brine, dried over Na₂SO₄, filtered and concentrated. The residue was purified by Prep-TLC (MeOH/EA=1:9) to get 24.5 mg of the title product as a yellow solid. MS (ES+): 656.9 [M+1]⁺.

¹HNMR Spectrum: (400 MHz, CDCl₃) δ 8.52 (s, 1H), 7.88-7.86 (d, J=8 Hz, 1H), 7.36 (s, 1H), 7.21 (s, 1H), 7.07-7.05 (d, J=8 Hz, 2H), 6.64-6.63 (d, J=4 Hz, 1H), 6.44-6.40 (d, J=16 Hz, 1H), 5.84-5.81 (m, 1H), 4.82-4.80 (m, 1H), 4.78-3.56 (m, 4H), 3.20 (s, 1H), 2.89 (s, 1H), 2.80-2.76 (d, J=16 Hz, 2H), 2.49 (s, 3H), 1.46 (m, 3H), 1.40-1.25 (m, 9H), 1.08-0.97 (m, 5H).

### EXAMPLE 14

### (S)-2⁴-(4-acryloyl-2-methylpiperazin-1-yl)-2⁶,3⁶-difluoro-1²-isopropyl-4-nicotinoyl-2¹,2²-dihydro-4-aza-2(1,7)-pyrido[2,3-d]pyrimidina-1(3,4)-pyridina-3(1,2)-benzenacycloheptaphan-2²-one

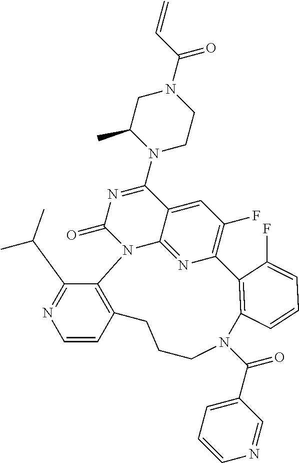 [SMILES: C=CC(=O)N1CCN(c2nc(=O)n3c4nc(c(F)cc24)-c2c(F)cccc2N(C(=O)c2cccnc2)CCCc2ccnc(C(C)C)c2-3)[C@@H](C)C1]

To a stirred solution of Example 2 (35 mg, 0.060 mmol) in NMP (3 ml) were added pyridine-3-carboxylic acid (29 mg, 0.239 mmol), 2-Chloro-1,3-dimethylimidazolidinium hexafluorophosphate (99 mg, 0.359 mmol) and DIPEA (46 mg, 0.359 mmol) at rt. After stirring at 80° C. overnight, the reaction mixture was quenched with water and extracted with EtOAc. The organic layer was washed with brine, dried over Na₂SO₄, filtered and concentrated. The residue was purified by Prep-TLC (MeOH/EA=1:9) to get 5.8 mg of the title product as a yellow solid. MS (ES+): 690.8 [M+1]⁺

¹HNMR Spectrum: (400 MHz, CDCl₃) δ 8.52-8.48 (m, 2H), 8.34 (s, 1H), 7.90-7.85 (m, 1H), 7.44-7.42 (d, J=8 Hz, 1H), 7.11 (s, 1H), 7.07-7.03 (m, 3H), 6.78 (s, 1H), 6.68 (s, 1H), 6.62-6.58 (m, 1H), 6.44-6.40 (d, J=16 Hz, 1H), 5.85-5.82 (d, J=12 Hz, 1H), 5.34-4.81 (m, 1H), 4.81-4.16 (m, 2H), 4.11-3.93 (m, 3H), 3.75-3.32 (m, 3H), 2.81-2.64 (m, 3H), 2.29-2.02 (m, 2H), 1.15-0.73 (m, 9H).

### EXAMPLE 15

### (S)-2⁴-(4-acryloyl-2-methylpiperazin-1-yl)-2⁶,3⁶-difluoro-4-(2-hydroxy-2-methylpropanoyl)-1²-isopropyl-2¹,2²-dihydro-4-aza-2(1,7)-pyrido[2,3-d]pyrimidina-1(3,4)-pyridina-3(1,2)-benzenacycloheptaphan-2²-one

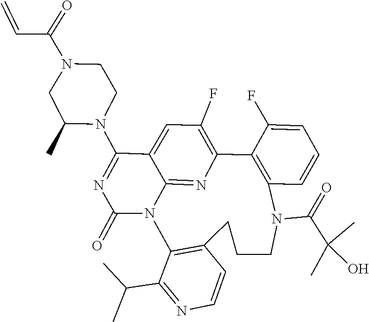 [SMILES: C=CC(=O)N1CCN(c2nc(=O)n3c4nc(c(F)cc24)-c2c(F)cccc2N(C(=O)C(C)(C)O)CCCc2ccnc(C(C)C)c2-3)[C@@H](C)C1]

To a stirred solution of Example 2 (40 mg, 0.0816 mmol), 2-hydroxy-2-methylpropanoic acid (25.6 mg, 0.245 mmol) and DIPEA (8064 mg, 0.49 mmol) in NMP (2 ml) was added CIP (45.6 mg, 0.164 mmol) at rt. The resulting mixture was stirred for 4 h at 50° C. and then quenched with water. The reaction mixture was extracted with EtOAc. The organic layer was washed with brine, dried over Na₂SO₄, filtered and concentrated. The residue was purified by Prep-TLC (EA:MeOH=5:1) to obtain 9 mg of the title product as a yellow solid. MS (ES+): 671.8 [M+1]⁺.

¹HNMR Spectrum: (400 MHz, CDCl₃) δ 8.52-8.50 (d, J=8 Hz, 1H), 7.85 (m, 1H), 7.44 (m, 1H), 7.20 (m, 1H), 7.16-7.04 (m, 2H), 6.62-6.55 (dd, 1H), 6.44-6.39 (d, J=20 Hz, 1H), 5.82-5.74 (m, 1H), 4.74 (s, 1H), 4.55-4.15 (m, 1H), 4.03-3.82 (m, 1H), 3.82-3.48 (m, 2H), 3.49 (s, 1H), 2.82 (m, 1H), 2.54-2.37 (m, 2H), 1.81 (s, 1H), 1.65 (s, 1H), 1.54-1.41 (m, 8H), 0.99-0.97 (d, J=8 Hz, 3H), 0.90-0.76 (m, 8H).

### EXAMPLE 16

### (S)-2⁴-(4-acryloyl-2-methylpiperazin-1-yl)-2⁶,3⁶-difluoro-1²-isopropyl-4-methyl-2¹,2²-dihydro-4,6-diaza-2(1,7)-pyrido[2,3-d]pyrimidina-1(3,4)-pyridina-3(1,2)-benzenacyclooctaphane-2²,5-dione

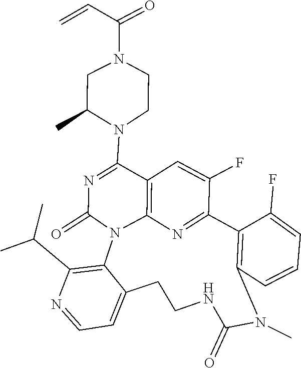 [SMILES: C=CC(=O)N1CCN(c2nc(=O)n3c4nc(c(F)cc24)-c2c(F)cccc2N(C)C(=O)NCCc2ccnc(C(C)C)c2-3)[C@@H](C)C1]

To a stirred solution of the product of example 5 (5.2 mg, 0.007 mmol) and cesium carbonate (27.5 mg, 0.084 mmol) in DMF (0.9 ml) was added iodomethane (48.0 mg, 0.336 mmol). The mixture was stirred at rt overnight, and then was quenched with water and extracted with EA. The organic layer was washed with brine, dried over Na₂SO₄, filtered and concentrated to get 3.7 mg of the title product as a yellow solid. MS (ES+): 628.8[M+1]⁺.

¹HNMR Spectrum: (400 MHz, CDCl₃) δ 8.57-8.56 (d, J=4 Hz, 1H), 7.85-7.83 (d, J=8 Hz, 1H), 7.52 (m, 1H), 7.16-7.11 (m, 3H), 6.65 (s, 1H), 6.44-6.40 (m, 1H), 5.84-5.81 (d, J=12 Hz, 1H), 5.36-5.33 (m, 1H), 4.78-4.46 (m, 3H), 4.21 (m, 1H), 4.01-3.64 (m, 1H), 3.58-3.20 (m, 1H), 3.12 (m, 4H), 2.85 (m, 1H), 2.62 (m, 1H), 2.21 (m, 1H), 2.01 (m, 1H), 1.47-1.41 (m, 3H), 1.29-1.25 (m, 4H), 1.01 (m, 3H).

### EXAMPLE 17

### (S)-2⁴-(4-acryloyl-2-methylpiperazin-1-yl)-2⁶,3⁶-difluoro-1⁶-isopropyl-2¹,2²-dihydro-4-aza-2(1,7)-pyrido[2,3-d]pyrimidina-1,3(1,2)-dibenzenacycloheptaphan-2²-one

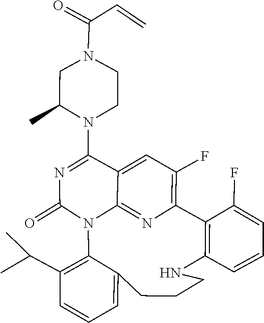 [SMILES: C=CC(=O)N1CCN(c2nc(=O)n3c4nc(c(F)cc24)-c2c(F)cccc2NCCCc2cccc(C(C)C)c2-3)[C@@H](C)C1]

### Step 1 2-bromo-6-isopropylaniline

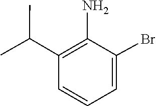

To a solution of 2-isopropylaniline (1.00 g, 7.4 mmol) in benzene (40.0 mL) was added NBS (1.31 g, 7.4 mmol) all at once. After stirring at rt overnight. The volume of the reaction mixture was deduced by one quarter under reduced pressure and the solid was removed by filtration. The filtrate was concentrated and pentane was added with ice followed by 0.4 mL acetic anhydride. After about 15 min, solid was filtered off. Pentane solution was separated from filtrate, washed with NH₄OH, and concentrated. The residue was distilled at 83° C. under 0.2 mmHg to give title compound (600.0 mg).

¹H NMR (400 MHz, DMSO-d₆): δ 7.22 (d, 1H), 7.04 (d, 1H), 6.53-6.49 (t, 1H), 4.98 (s, 2H), 3.07-3.03 (m, 1H), 6.15 (d, 6H).

### Step 2 tert-butyl (E)-3-(2-amino-3-isopropylphenyl)acrylate

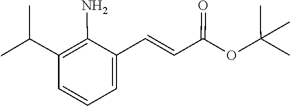 [SMILES: CC(C)c1cccc(/C=C/C(=O)OC(C)(C)C)c1N]

A solution of 2-bromo-6-isoproplyaniline (50.0 mg, 4.70 mmol), tert-butyl acrylate (59.6 mg, 0.467 mmol), K₂CO₃ (64.0 mg, 0.46 mmol) and Pd(OAc)₂ (10.0 mg, 0.045 mmol), tri-o-tolylphosphane (28.0 mg, 0.093 mmol) in DMF (15 mL) was heated at 100° C. under Ar atmosphere for 2 h. The reaction was quenched with water and extracted with EtOAc (10 mL×2). The combined organic phase was dried over Na₂SO₄, and concentrated. The residue was purified by silica gel chromatography eluted with a gradient of EtOAc/Hexane (0-10%) to afford title compound (40 mg). MS (ESI+): 262.1 [M+1]⁺.

¹H NMR (400 MHz, DMSO-d₆): δ 7.86 (d, 1H), 7.27 (d, 1H), 7.07 (d, 1H), 6.58-6.54 (t, 1H), 6.21 (d, 1H), 5.23 (s, 2H), 3.06-3.00 (m, 1H), 1.48 (s, 9H), 1.13 (d, 6H).

### Step 3 tert-butyl-(E)-3-(2-(3-(2,6-dichloro-5-fluoronicotinoyl)ureido)-3-isopropylphenyl)acrylate

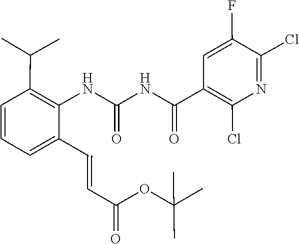 [SMILES: CC(C)c1cccc(/C=C/C(=O)OC(C)(C)C)c1NC(=O)NC(=O)c1cc(F)c(Cl)nc1Cl]

Starting with 2,6-dichloro-5-fluoronicotinamide and the product of Step 2, the title product was obtained as a yellow solid by following the procedure described in Step 3 of Example 1. MS (ESI+): 496.1 [M+1]⁺.

¹H NMR (400 MHz, DMSO-d₆): δ 11.37 (s, 1H), 9.60 (s, 1H), 8.52 (d, 1H), 7.72-7.66 (m, 2H), 7.44 (d, 1H), 7.38-7.34 (t, 1H), 6.47 (d, 1H), 3.31-3.12 (m, 1H), 1.47 (s, 9H), 1.17 (d, 6H).

### Step 4 tert-butyl-(E)-3-(2-(7-chloro-6-fluoro-2,4-dioxo-3,4-dihydropyrido[2,3-d]pyrimidin-1(2H)-yl)-3-isopropylphenyl)acrylate

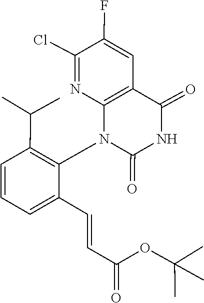 [SMILES: CC(C)c1cccc(/C=C/C(=O)OC(C)(C)C)c1-n1c(=O)[nH]c(=O)c2cc(F)c(Cl)nc21]

Starting with the product od Step 3, the title product was obtained as a yellow solid by following the procedure described in Step 3 of Example 8.

¹HNMR (400 MHz, CDCl3-d1): δ 8.55 (s, 1H), 8.24 (d, 1H), 7.64-7.62 (dd, 1H), 7.55-7.50 (m, 2H), 7.25 (d, 1H), 6.36 (d, 1H), 2.58-2.54 (m, 1H), 1.45 (s, 9H), 1.18 (d, 3H), 1.07 (d, 3H).

### Step 5 2-fluoro-6-nitrophenyl trifluoromethanesulfonate

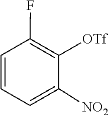 [SMILES: O=[N+]([O-])c1cccc(F)c1OS(=O)(=O)C(F)(F)F]

To a solution of 2-fluoro-6-nitrophenol (10.0 g, 63.69 mmol) in DCM (200.0 mL) cooled at 0° C., were added pyridine (6.15 mL, 76.43 mmol) and Tf₂O (12.86 mL, 76.43 mmol). After stirring for 2 h, the reaction was quenched with NaHCO₃(aq) and extracted with DCM. The DCM layer was washed with 1N HCl, dried over MgSO₄ and concentrated to give the crude title compound (17.0 g).

¹HNMR (400 MHz, CDCl₃): δ 8.00-7.97 (m, 1H), 7.64-7.54 (m, 2H).

### Step 6 2-(2-fluoro-6-nitrophenyl)-4,4,5,5-tetramethyl-1,3,2-dioxaborolane

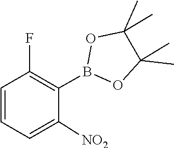 [SMILES: CC1(C)OB(c2c(F)cccc2[N+](=O)[O-])OC1(C)C]

To a solution of the product of Step 5 (290.0 mg, 1.0 mmol) in dioxane (3.0 mL), was added 4,4,4′,4′,5,5,5′,5′-octamethyl-2,2′-bi(1,3,2-dioxaborolane) (381.0 mg, 1.50 mmol), KOAc (295.0 mg, 3.0 mmol) and Pd(dppf)Cl₂ (82.0 mg, 0.1 mmol) and 4 drops of water. After stirring at 80° C. for 2.5 h, the reaction mixture was filtered through a pad of celite. The filtrate was concentrated and the residue was purified by silica gel chromatography eluted with a gradient of EtOAc/Hexane (0-20%) to afford the title compound (250.0 mg).

¹HNMR (400 MHz, CDCl3): δ 8.03 (d, 1H), 7.56-7.50 (m, 1H), 7.39-7.35 (t, 1H), 1.45 (s, 12H).

### Step 7 3-fluoro-2-(4,4,5,5-tetramethyl-1,3,2-dioxaborolan-2-yl)aniline

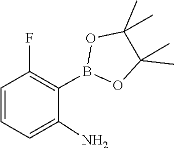 [SMILES: CC1(C)OB(c2c(N)cccc2F)OC1(C)C]

To a solution of 2-(2-fluoro-6-nitrophenyl)-4,4,5,5-tetramethyl-1,3,2-dioxaborolane (2.49 g, 9.32 mmol) in EtOAc (40 mL) was added 10% Pd/C (992 mg, 0.93 mmol) under nitrogen atmosphere. The mixture was then stirred under H₂ atmosphere at rt for 16.5 h. The solution was filtered through a pad of celite, and the pad was washed with ethyl acetate. The filtrate was concentrated to give the title compound (2.2 g). MS (ESI+): 238.1 [M+1]⁺.

### Step 8 tert-butyl-(E)-3-(2-(7-(2-amino-6-fluorophenyl)-6-fluoro-2,4-dioxo-3,4-dihydropyrido[2,3-d]pyrimidin-1(2H)-yl)-3-isopropylphenyl)acrylate

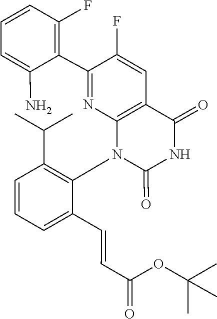 [SMILES: CC(C)c1cccc(/C=C/C(=O)OC(C)(C)C)c1-n1c(=O)[nH]c(=O)c2cc(F)c(-c3c(N)cccc3F)nc21]

To a solution of the product of Step 4 (100.0 mg, 0.218 mmol) and Step 7 (155.0 mg, 0.654 mmol) in dioxane (3.0 mL), were added K₂CO₃ (60.0 mg, 0.436 mmol) and Pd(dppf)Cl₂ (18.0 mg, 0.022 mmol) under Ar atmosphere. After stirring at 80° C. for 2 h, the reaction was quenched with water (10 mL) and extracted with EtOAc (10 mL×2). The combined extracts were dried over Na₂SO₄, filtrated and concentrated. The residue was purified by silica gel chromatography eluted with a gradient of EtOAc/Hexane (0-20%) to afford the title compound (120 mg). MS (ESI+): 479.1 [M−55]⁺.

### Step 9 tert-butyl-3-(2-(7-(2-amino-6-fluorophenyl)-6-fluoro-2,4-dioxo-3,4-dihydropyrido[2,3-d]pyrimidin-1(2H)-yl)-3-isopropylphenyl)propanoate

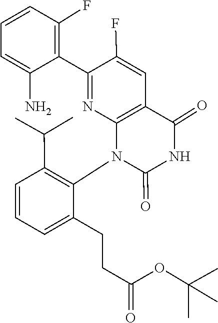 [SMILES: CC(C)c1cccc(CCC(=O)OC(C)(C)C)c1-n1c(=O)[nH]c(=O)c2cc(F)c(-c3c(N)cccc3F)nc21]

To a solution of the product of Step 8 (120.0 mg, 0.225 mmol) in MeOH (5 mL) and 7M NH₃/MeOH (0.05 mL) was added Pd/C (24 mg, 0.023 mmol). The mixture was stirred at rt for 2 hour and then filtered through a pad of celite and the filtrate was concentrated to afford the title compound (118.0 mg). MS (ESI+): 481.1 [M−55]⁺.

### Step 10 3-(2-(7-(2-amino-6-fluorophenyl)-6-fluoro-2,4-dioxo-3,4-dihydropyrido[2,3-d]pyrimidin-1(2H)-yl)-3-isopropylphenyl)propanoic acid

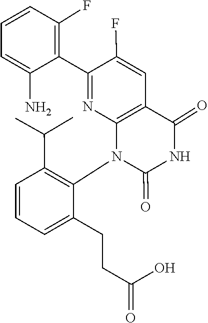 [SMILES: CC(C)c1cccc(CCC(=O)O)c1-n1c(=O)[nH]c(=O)c2cc(F)c(-c3c(N)cccc3F)nc21]

To a solution of 4N HCl/dioxane (4.0 mL) was the product of Step 9 (118.0 mg, 0.22 mmol) at 0° C. After stirring at 0° C. for 10 min, the reaction mixture was kept at rt. for 2 h. The reaction mixture was concentrated to dryness for to afford the crude title compound (100.0 mg). MS (ESI+): 481.1 [M+1]⁺.

### Step 11 2⁶,3⁶-difluoro-1⁶-isopropyl-2¹,2²,2³,2⁴-tetrahydro-4-aza-2(1,7)-pyrido[2,3-d]pyrimidina-1,3(1,2)-dibenzenacycloheptaphane-2²,2⁴,5-trione

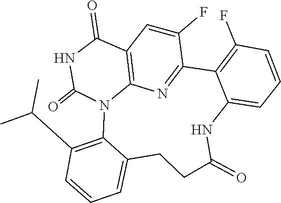 [SMILES: CC(C)c1cccc2c1-n1c(=O)[nH]c(=O)c3cc(F)c(nc31)-c1c(F)cccc1NC(=O)CC2]

Starting with the product of Step 10, the title product was obtained as a yellow solid by following the procedure described in Step 7 of Example 1. MS (ESI+): 463.1 [M+1]⁺.

### Step 122⁶,3⁶-difluoro-1⁶-isopropyl-2¹,2²,2³,2⁴-tetrahydro-4-aza-2(1,7)-pyrido[2,3-d]pyrimidina-1,3(1,2)-dibenzenacycloheptaphane-2²,2⁴-dione

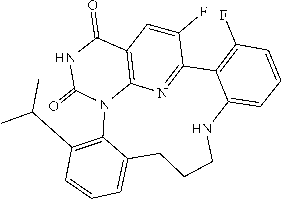 [SMILES: CC(C)c1cccc2c1-n1c(=O)[nH]c(=O)c3cc(F)c(nc31)-c1c(F)cccc1NCCC2]

Starting with the product of Step 11, the title product was obtained as a yellow solid by following the procedure described in Step 1 of Example 2. MS (ESI+): 449.1 [M+1]⁺.

### Step 13(S)-2⁴-(4-acryloyl-2-methylpiperazin-1-yl)-2⁶,3⁶-difluoro-1⁶-isopropyl-2¹,2²-dihydro-4-aza-2(1,7)-pyrido[2,3-d]pyrimidina-1,3(1,2)-dibenzenacycloheptaphan-2²-one

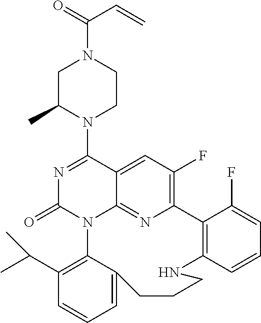 [SMILES: C=CC(=O)N1CCN(c2nc(=O)n3c4nc(c(F)cc24)-c2c(F)cccc2NCCCc2cccc(C(C)C)c2-3)[C@@H](C)C1]

To a solution of the product Step 12 (10.0 mg, 0.022 mmol) and DIPEA (28.4 mg, 0.22 mmol) in CH₃CN (1.0 mL) was added POCl₃ (20 mg, 0.134 mmoL) at rt under Ar. After stirring at 80° C. for 1 hour, the reaction mixture was then concentrated to dryness. The residue was dissolved in DMF (1.0 mL) and the resulted solution was treated with DIPEA (28.4 mg, 0.22 mmol) and Intermediate 2 (13.0 mg, 0.044 mmol). After stirring at rt overnight, EtOAc (10 mL) was added and the EtOAc layer was washed with water (8 mL×3), brine (8 mL×2), dried over Na₂SO₄, and concentrated. The residue was purified by silica gel chromatography eluted with a gradient of MeOH/DCM (0-5%) to afford the title compound (6.0 mg). MS (ESI+): 585.1 [M+1]⁺.

¹HNMR (400 MHz, CDCl₃): δ 7.88-7.81 (m, 1H), 7.41-7.37 (t, 1H), 7.29-7.20 (m, 3H), 7.69-7.59 (m, 1H), 6.55-6.47 (m, 2H), 6.43-6.39 (m, 1H), 5.82 (d, 1H), 4.87-4.73 (m, 1H), 4.68-4.56 (m, 1H), 4.18-4.13 (m, 1H), 3.97-3.87 (m, 1H), 3.78-3.68 (m, 1H), 3.56-3.36 (m, 1H), 3.30-3.23 (m, 1H), 3.00-2.94 (m, 1H), 2.88-2.84 (m, 1H), 2.67-2.61 (m, 1H), 2.41-2.31 (m, 2H), 2.03-1.99 (m, 2H), 1.23-1.22 (m, 3H), 1.21-1.20 (m, 3H), 0.95-0.94 (m, 3H).

### EXAMPLE 18

### (S)-2⁴-(4-acryloyl-2-methylpiperazin-1-yl)-2⁶,3⁶-difluoro-1⁶-isopropyl-2¹,2²-dihydro-4-aza-2(1,7)-pyrido[2,3-d]pyrimidina-1,3(1,2)-dibenzenacycloheptaphane-2²,5-dione

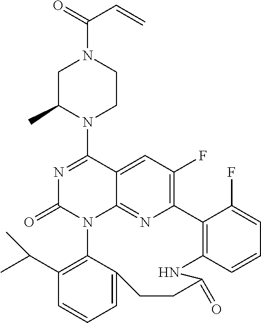 [SMILES: C=CC(=O)N1CCN(c2nc(=O)n3c4nc(c(F)cc24)-c2c(F)cccc2NC(=O)CCc2cccc(C(C)C)c2-3)[C@@H](C)C1]

Starting with the product of Step 11 of Example 17 and Intermediate 2, the title product was obtained as a yellow solid by following the procedure described in Step 13 of Example 17. MS (ESI+): 599.1 [M+1]⁺.

¹HNMR (400 MHz, CDCl3-d1): δ 7.89-7.80 (m, 1H), 7.73-7.70 (m, 1H), 7.53-7.33 (m, 4H), 7.16-7.10 (m, 1H), 7.03-6.97 (m, 1H), 6.86-6.77 (m, 1H), 6.69-6.51 (m, 1H), 6.45-6.38 (m, 1H), 5.84-5.81 (m, 1H), 4.33-3.98 (m, 4H), 3.77-3.60 (m, 2H), 3.09-2.98 (m, 1H), 2.93-2.85 (m, 1H), 2.82-2.76 (m, 1H), 2.63-2.57 (m, 1H), 2.37-2.33 (m, 1H), 2.22-2.20 (m, 1H), 1.22-1.06 (m, 6H), 0.90-0.89 (m, 3H).

### EXAMPLE 19

### (S)-2⁴-(4-acryloyl-2-methylpiperazin-1-yl)-2⁶,3⁶-difluoro-1⁶-isopropyl-4-methyl-2¹,2²-dihydro-4-aza-2(1,7)-pyrido[2,3-d]pyrimidina-1,3(1,2)-dibenzenacycloheptaphan-2²-one

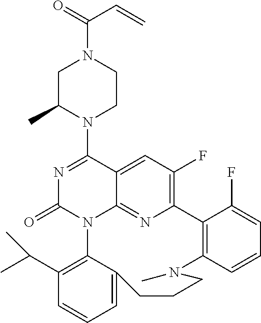 [SMILES: C=CC(=O)N1CCN(c2nc(=O)n3c4nc(c(F)cc24)-c2c(F)cccc2N(C)CCCc2cccc(C(C)C)c2-3)[C@@H](C)C1]

To a solution of the product of Example 17 (25 mg, 0.043 mmol) in DMF (1.0 mL) were added K₂CO₃ (11.8 mg, 0.086 mmol) and CH₃I (18.2 mg, 0.13 mmol). After the reaction mixture was stirred at 40° C. for 3 h, and additional CH₃I (36.4 mg, 0.26 mmol) was added and the reaction mixture was stirred at rt overnight. Water (5 mL) was added and the mixture was extracted with EtOAc (10 mL). The organic layer was washed with water (5 mL×3), brine (5 mL), dried over Na₂SO₄, and filtered. The filtrate was concentrated and the residue was purified by silica gel chromatography eluted with a gradient of MeOH/DCM (0-3%) to afford the title compound (10 mg, 39% yield). MS (ESI+): 599.1 [M+1]⁺.

¹HNMR (400 MHz, CDCl3-d1): δ 7.75-7.73 (m, 1H), 7.34-26 (m, 3H), 7.14-7.12 (m, 1H), 7.02-7.00 (m, 1H), 6.88-6.83 (m, 1H), 6.68-6.54 (m, 1H), 6.41 (d, 1H), 5.81 (d, 1H), 5.40-5.33 (m, 1H), 4.90-4.75 (m, 1H), 4.68-4.50 (m, 1H), 4.19-4.14 (m, 1H), 3.98-3.86 (m, 1H), 3.81-3.71 (m, 1H), 3.59-3.50 (m, 1H), 3.46-3.38 (m, 1H), 3.26-3.12 (m, 1H), 3.06-3.00 (m, 1H), 2.81-2.74 (m, 1H), 2.59-2.54 (m, 1H), 2.44-2.42 (m, 4H), 1.94-1.85 (m, 1H), 1.25-1.23 (m, 3H), 0.98-0.96 (m, 3H).

### EXAMPLE 20

### 2⁴-((2S,5R)-4-acryloyl-2,5-dimethylpiperazin-1-yl)-2⁶,3⁶-difluoro-1⁶-isopropyl-2¹,2²-dihydro-4-aza-2(1,7)-pyrido[2,3-d]pyrimidina-1,3(1,2)-dibenzenacycloheptaphan-2²-one

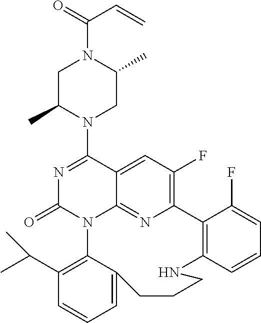 [SMILES: C=CC(=O)N1C[C@H](C)N(c2nc(=O)n3c4nc(c(F)cc24)-c2c(F)cccc2NCCCc2cccc(C(C)C)c2-3)C[C@H]1C]

### Step 1 2⁴-((2S,5R)-4-(3-chloropropanoyl)-2,5-dimethylpiperazin-1-yl)-2⁶,3⁶-difluoro-1⁶-isopropyl-2¹,2²-dihydro-4-aza-2(1,7)-pyrido[2,3-d]pyrimidina-1,3(1,2)-dibenzenacycloheptaphan-2²-one

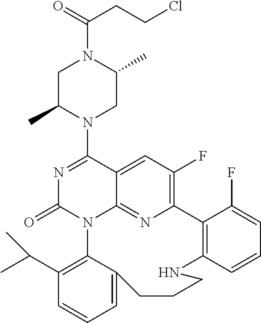 [SMILES: CC(C)c1cccc2c1-n1c(=O)nc(N3C[C@@H](C)N(C(=O)CCCl)C[C@@H]3C)c3cc(F)c(nc31)-c1c(F)cccc1NCCC2]

Starting from the product of Step 12 of Example 17 (25 mg) and Intermediate 4 (46 mg), the title product was obtained as a yellow solid by following the procedure described in Step 13 of Example 17. MS (ESI+): 635.1 [M+1]⁺.

### Step 2 2⁴-((2S,5R)-4-acryloyl-2,5-dimethylpiperazin-1-yl)-2⁶,3⁶-difluoro-1⁶-isopropyl-2¹,2²-dihydro-4-aza-2(1,7)-pyrido[2,3-d]pyrimidina-1,3(1,2)-dibenzenacycloheptaphan-2²-one

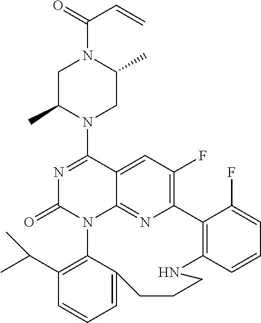 [SMILES: C=CC(=O)N1C[C@H](C)N(c2nc(=O)n3c4nc(c(F)cc24)-c2c(F)cccc2NCCCc2cccc(C(C)C)c2-3)C[C@H]1C]

To a solution of the product of Step 1 (20 mg) in CH₃CN (1.0 mL) was added Et₃N (112.7 mg). After stirring at 80° C. for 12 h, the reaction mixture was diluted with EtOAc (10 mL) and washed with water (8 mL×2), brine (8 mL), and dried over Na₂SO₄. The solution was concentrated to afford the title compound (20 mg). MS (ESI+): 599.1 [M+1]⁺.

### EXAMPLE 20A and 20B

Example 20 (˜20 mg) was separated by silica gel chromatography eluted with a gradient of MeOH/DCM (0-5%) to afford Example 20A (8 mg, fast eluted) and Example 20B (7.0 mg, slow eluted) as yellow solid.

Example 20A: ¹HNMR (400 MHz, CDCl): δ 7.83-7.78 (m, 1H), 7.41-7.38 (m, 1H), 7.28-7.20 (m, 3H), 6.61-6.47 (m, 3H), 6.44-6.35 (t, 1H), 5.81-5.78 (m, 1H), 5.12-5.10 (m, 1H), 5.01-4.97 (m, 1H), 4.41-4.30 (m, 1H), 4.15-4.08 (m, 1H), 4.00-3.00 (m, 2H), 3.72-3.64 (m, 1H), 3.32-3.29 (m, 1H), 3.01-2.96 (m, 1H), 2.89-2.82 (m, 1H), 2.66-2.58 (m, 1H), 2.44-2.32 (m, 2H), 2.02-1.99 (m, 1H), 1.40-1.39 (m, 3H), 1.22-1.17 (m, 6H), 0.91-0.89 (m, 3H).

MS (ESI+): 599.1 [M+1]⁺.

Example 20B: ¹HNMR (400 MHz, CDCl): δ 7.89-7.82 (m, 1H), 7.40-7.36 (m, 1H), 7.28-7.19 (m, 3H), 6.66-6.55 (m, 1H), 6.53-6.40 (m, 3H), 5.83-5.78 (m, 1H), 5.20-5.13 (m, 1H), 4.94-4.88 (m, 1H), 4.80-4.74 (m, 1H), 4.54-4.46 (m, 1H), 4.44-4.39 (m, 1H), 3.74-3.63 (m, 2H), 3.58-3.53 (m, 1H), 3.31-3.26 (m, 1H), 3.22-3.19 (m, 1H), 3.00-2.95 (m, 1H), 2.89-2.84 (m, 1H), 2.64-2.58 (m, 1H), 2.38-2.33 (m, 1H), 2.00-1.97 (m, 1H), 1.64-1.62 (m, 3H), 1.50-1.48 (m, 3H), 1.22-1.17 (m, 3H), 1.00-0.96 (m, 3H).

MS (ESI+): 599.1 [M+1]⁺.

### EXAMPLE 21

### (S)-2⁴-(4-acryloyl-2-methylpiperazin-1-yl)-2⁶,3⁶-difluoro-1⁶-isopropyl-2¹,2²-dihydro-4,8-diaza-2(1,7)-pyrido[2,3-d]pyrimidina-1,3(1,2)-dibenzenacyclooctaphane-2²,5-dione

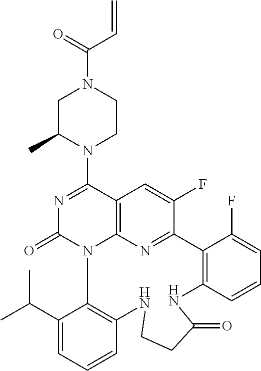 [SMILES: C=CC(=O)N1CCN(c2nc(=O)n3c4nc(c(F)cc24)-c2c(F)cccc2NC(=O)CCNc2cccc(C(C)C)c2-3)[C@@H](C)C1]

### Step 1 tert-butyl 3-((3-bromo-2-nitrophenyl)amino)propanoate

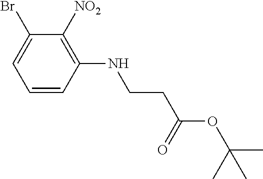 [SMILES: CC(C)(C)OC(=O)CCNc1cccc(Br)c1[N+](=O)[O-]]

A solution of 1-bromo-3-fluoro-2-nitrobenzene (0.6918 g, 3.145 mmol), tert-butyl 3-aminopropanoate hydrochloride (0.6436 g, 3.543 mmol) and DIPEA (1.55 mL, 9.38 mmol) in 15 mL DMA was stirred for 23 h at 80° C. Water (80 mL) was added and the mixture was extracted with EtOAc (4×20 mL). The combined organic layer was washed with water (3×20 mL), brine, dried over Na₂SO₄, filtered, concentrated to give the title compound as a yellow oil (1.0 g).

¹HNMR (400 MHz, CDCl₃) δ 7.15 (t, J=8.2 Hz, 1H), 6.96 (dd, J=7.8, 0.9 Hz, 1H), 6.77 (d, J=8.4 Hz, 1H), 5.84 (s, 1H), 3.46 (dd, J=12.3, 6.3 Hz, 2H), 2.55 (t, J=6.5 Hz, 2H), 1.47 (s, 9H).

### Step 2 tert-butyl 3-((3-bromo-2-nitrophenyl)(tert-butoxycarbonyl)amino)propanoate

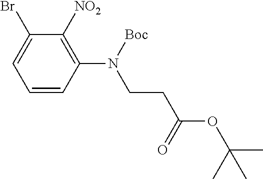 [SMILES: CC(C)(C)OC(=O)CCN(C(=O)OC(C)(C)C)c1cccc(Br)c1[N+](=O)[O-]]

To a solution of the product od Step 1 (0.4405 g, 1.276 mmol), 4-dimethylaminopyridine (0.1614 g, 1.321 mmol) and DIPEA (0.86 mL, 5.22 mmol) in 5 mL dry DMF was added di-tert-butyl dicarbonate (1.20 mL, 5.22 mmol) at rt. The reaction mixture was stirred for 23 h at 80° C.

Water (80 mL) was added and the reaction mixture was extracted with EtOAc (4×20 mL). The combined organic layer was washed with water (3×20 mL), brine, dried over Na₂SO₄, filtered, concentrated. The residue was purified by silica gel column chromatography (eluent: EtOAc:Hexanes/0:100 to 10:90) to give the title compound (0.3688 g) as a brown oil. MS (ESI+): 467.1, 469.1 [M+23]⁺.

### Step 3 tert-butyl 3-((tert-butoxycarbonyl)(2-nitro-3-(prop-1-en-2-yl)phenyl)amino)propanoate

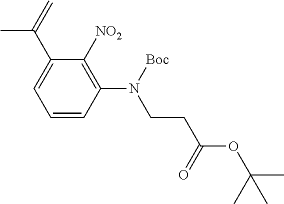 [SMILES: C=C(C)c1cccc(N(CCC(=O)OC(C)(C)C)C(=O)OC(C)(C)C)c1[N+](=O)[O-]]

A mixture of the product of Step 2 (0.36 g, 1.04 mmol), isopropenylboronic acid pinacol ester (0.45 mL, 2.39 mmol), tetrakis(triphenylphosphine)palladium (0.0956 g, 0.0827 mmol) and K₂CO₃ (0.4138 g, 2.994 mmol) in 20 mL 1,4-dioxane and 4 mL H₂O was stirred for 3 h at 80° C. under Argon. EtOAc (20 mL) and 20 mL brine were added. The organic layer was dried over Na₂SO₄, filtered, concentrated and purified by silica gel column chromatography eluted with a gradient of petroleum ether/EtOAc (100:0 to 90:10) to give the title compound (0.2838 g) as a red oil. MS (ESI+): 429.2 [M+23]⁺.

### Step 4 tert-butyl 3-((2-amino-3-isopropylphenyl)(tert-butoxycarbonyl)amino)propanoate

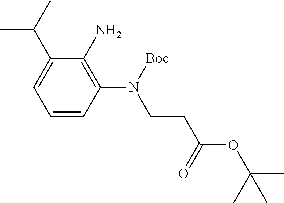 [SMILES: CC(C)c1cccc(N(CCC(=O)OC(C)(C)C)C(=O)OC(C)(C)C)c1N]

A mixture of the product of Step 3 (0.28 g, 0.69 mmol) and 10% Pd/C (0.164 g, 0.155 mmol) in 25 mL EtOAc was stirred for 1 h at rt with a balloon of H₂. The reaction mixture was filtered through a Celite pad, and the filtrate was concentrated. To the residue was dissolved in 20 mL Methanol and treated with Raney Ni (3.1 g). The reaction mixture was stirred for 2 h at 30° C. with a balloon of H₂. The reaction mixture was filtered through a Celite pad, and the filtrate was concentrated to give the title compound (0.2514 g) as a colorless oil. MS (ESI+): 379.2 [M+1]⁺.

### Step 5 tert-butyl 3-((tert-butoxycarbonyl)(2-(3-(2,6-dichloro-5-fluoronicotinoyl)ureido)-3-isopropylphenyl)amino)propanoate

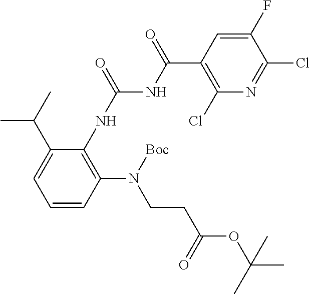 [SMILES: CC(C)c1cccc(N(CCC(=O)OC(C)(C)C)C(=O)OC(C)(C)C)c1NC(=O)NC(=O)c1cc(F)c(Cl)nc1Cl]

Starting with 2,6-dichloro-5-fluoronicotinamide (3.67 g) and the product of Step 4 (3.58 g), the title product was obtained as a white solid (3.8 g) by following the procedure described in Step 3 of Example 1. MS (ESI+): 611.2 [M−1]⁺.

### Step 6 tert-butyl 3-((tert-butoxycarbonyl)(2-(7-chloro-6-fluoro-2,4-dioxo-3,4-dihydropyrido[2,3-d]pyrimidin-1(2H)-yl)-3-isopropylphenyl)amino)propanoate

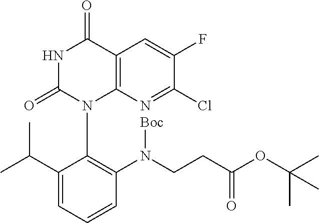 [SMILES: CC(C)c1cccc(N(CCC(=O)OC(C)(C)C)C(=O)OC(C)(C)C)c1-n1c(=O)[nH]c(=O)c2cc(F)c(Cl)nc21]

Starting with 2,6-dichloro-5-fluoronicotinamide (3.67 g) and the product of Step 5 (3.35 g), the title product was obtained as a white solid (1.94 g) by following the procedure described in Step 3 of Example 8. MS (ESI+): 575.1 [M−1]⁺.

### Step 7 3-((2-(7-chloro-6-fluoro-2,4-dioxo-3,4-dihydropyrido[2,3-d]pyrimidin-1(2H)-yl)-3-isopropylphenyl)amino)propanoic acid

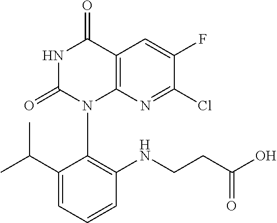 [SMILES: CC(C)c1cccc(NCCC(=O)O)c1-n1c(=O)[nH]c(=O)c2cc(F)c(Cl)nc21]

A solution of the product of Step 6 (0.8295 g, 1.437 mmol) in TFA (10.0 mL) and dichloromethane (10 mL) was stirred for 5 h at rt. Toluene (10 mL) was added and the mixture was concentrated to give the title compound as a grey solid (0.80 g). MS (ESI+): 421.1 [M+1]⁺.

### Step 8 3-((2-(7-(2-amino-6-fluorophenyl)-6-fluoro-2,4-dioxo-3,4-dihydropyrido[2,3-d]pyrimidin-1(2H)-yl)-3-isopropylphenyl)amino)propanoic acid

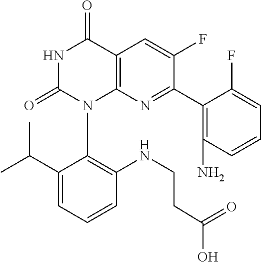 [SMILES: CC(C)c1cccc(NCCC(=O)O)c1-n1c(=O)[nH]c(=O)c2cc(F)c(-c3c(N)cccc3F)nc21]

Starting with the product of Step 7 (0.6 g) and 3-fluoro-2-(4,4,5,5-tetramethyl-1,3,2-dioxaborolan-2-yl)aniline (0.50 g), the title product was obtained as a white solid (0.66 g) by following the procedure described in Step 8 of Example 17. MS (ESI+): 496.2 [M+1]⁺.

### Step 9 2⁶,3⁶-difluoro-1⁶-isopropyl-2¹,2²,2³,2⁴-tetrahydro-4,8-diaza-2(1,7)-pyrido[2,3-d]pyrimidina-1,3(1,2)-dibenzenacyclooctaphane-2²,2⁴,5-trione

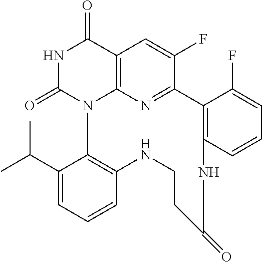 [SMILES: CC(C)c1cccc2c1-n1c(=O)[nH]c(=O)c3cc(F)c(nc31)-c1c(F)cccc1NC(=O)CCN2]

Starting with the product of Step 8 (0.24 g), the title product was obtained as a grey solid (0.11 g) by following the procedure described in Step 7 of Example 1. MS (ESI+): 632.0 [M+1]⁺.

### Step 10 (S)-2⁴-(4-acryloyl-2-methylpiperazin-1-yl)-2⁶,3⁶-difluoro-1⁶-isopropyl-2¹,2²-dihydro-4,8-diaza-2(1,7)-pyrido[2,3-d]pyrimidina-1,3(1,2)-dibenzenacyclooctaphane-2²,5-dione

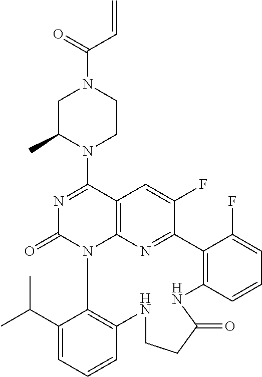 [SMILES: C=CC(=O)N1CCN(c2nc(=O)n3c4nc(c(F)cc24)-c2c(F)cccc2NC(=O)CCNc2cccc(C(C)C)c2-3)[C@@H](C)C1]

Starting with the product of Step 9 (44 mg) and Intermediate 1 (69 mg), the title product was obtained as a white solid (12 mg) by following the procedure described in Step 13 of Example 17, except the crude product was purified by reverse phase column chromatography (C-18, grain size: 40 m, pore size distribution: 120 A) eluted with CH₃CN/H₂O (10-80%). MS (ESI+): 614.3 [M+1]⁺.

### EXAMPLE 22

### (S)-2⁴-(4-acryloyl-2-methylpiperazin-1-yl)-2⁶,3⁶-difluoro-1⁶-isopropyl-2¹,2²-dihydro-4,8-diaza-2(1,7)-pyrido[2,3-d]pyrimidina-1,3(1,2)-dibenzenacyclooctaphan-2²-one

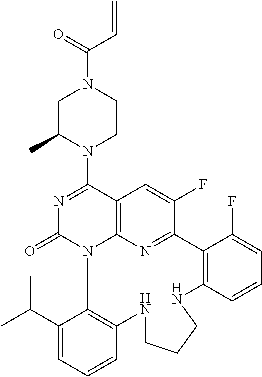 [SMILES: C=CC(=O)N1CCN(c2nc(=O)n3c4nc(c(F)cc24)-c2c(F)cccc2NCCCNc2cccc(C(C)C)c2-3)[C@@H](C)C1]

### Step 1 2⁶,3⁶-difluoro-1⁶-isopropyl-2¹,2²,2³,2⁴-tetrahydro-4,8-diaza-2(1,7)-pyrido[2,3-d]pyrimidina-1,3(1,2)-dibenzenacyclooctaphane-2²,2⁴-dione

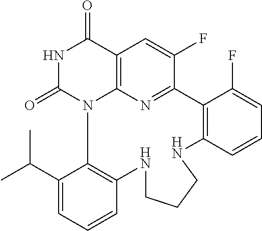 [SMILES: CC(C)c1cccc2c1-n1c(=O)[nH]c(=O)c3cc(F)c(nc31)-c1c(F)cccc1NCCCN2]

Starting with the product of Step 9 of Example 21 (190 mg), the title product was obtained as a yellow solid (70 mg) by following the procedure described in Step 7 of Example 1. MS (ESI+): 464.2 [M+1]⁺.

### Step 2 (S)-2⁴-(4-acryloyl-2-methylpiperazin-1-yl)-2⁶,3⁶-difluoro-1⁶-isopropyl-2¹,2²-dihydro-4,8-diaza-2(1,7)-pyrido[2,3-d]pyrimidina-1,3(1,2)-dibenzenacyclooctaphan-2²-one

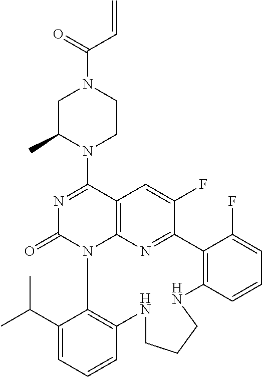 [SMILES: C=CC(=O)N1CCN(c2nc(=O)n3c4nc(c(F)cc24)-c2c(F)cccc2NCCCNc2cccc(C(C)C)c2-3)[C@@H](C)C1]

Starting with the product of Step 1 (50 mg) and Intermediate 1 (101 mg), the title product was obtained as a white solid (2.3 mg) by following the procedure described in Step 13 of Example 17, except the crude product was purified by reverse phase column chromatography (C-18, grain size: 40 m, pore size distribution: 120 A) eluted with CH₃CN/H₂O (10-80%). MS (ESI+): 600.3 [M+1]⁺.

### EXAMPLE 23A and 23B

### 2⁴-((2S,5R)-4-acryloyl-2,5-dimethylpiperazin-1-yl)-2⁶,3⁶-difluoro-1⁶-isopropyl-2¹,2²-dihydro-4,8-diaza-2(1,7)-pyrido[2,3-d]pyrimidina-1,3(1,2)-dibenzenacyclooctaphan-2²-one

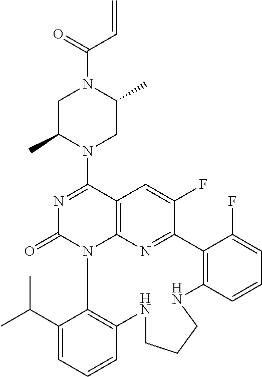 [SMILES: C=CC(=O)N1C[C@H](C)N(c2nc(=O)n3c4nc(c(F)cc24)-c2c(F)cccc2NCCCNc2cccc(C(C)C)c2-3)C[C@H]1C]

To a solution of the product of Step 1 of Example 22 (0.0598 g, 0.125 mmol) and DIPEA (0.22 mL, 1.26 mmol) in 5 mL dry MeCN was added POCl₃ (0.0800 mL, 0.858 mmol). The reaction mixture was stirred for 1 hour under reflux. MeCN was evaporated off and the residue was treated with a solution of Intermediate 3 (0.0738 g, 0.262 mmol), DIPEA (0.24 mL, 1.38 mmol) in 5 mL dry MeCN. The reaction mixture was stirred for 1 hour and then concentrated. The residue was first purified by gel silica column chromatography eluted with DCM/MeOH (100:0 to 90:10), then by reverse phase column chromatography (C-18, grain size: 40 m, pore size distribution: 120 Å) eluted with CH₃CN/H₂O (10-80%) to give Example 23A (0.0070 g, fast eluted) and Example 23B (0.0074 g, slow eluted) as yellow solids. MS (ESI+): 614.3 [M+1]⁺.

### EXAMPLE 24A and 24B

### 2⁴-((2S,5R)-4-acryloyl-2,5-dimethylpiperazin-1-yl)-2⁶,3⁶-difluoro-1⁶-isopropyl-2¹,2²-dihydro-4,8-diaza-2(1,7)-pyrido[2,3-d]pyrimidina-1,3(1,2)-dibenzenacyclooctaphane-2²,5-dione

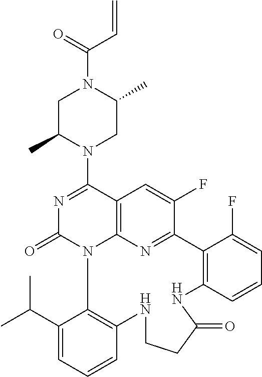 [SMILES: C=CC(=O)N1C[C@H](C)N(c2nc(=O)n3c4nc(c(F)cc24)-c2c(F)cccc2NC(=O)CCNc2cccc(C(C)C)c2-3)C[C@H]1C]

To a solution of the product of Step 9 of Example 21 (0.0470 g, 0.0984 mmol) and DIPEA (0.18 mL, 1.03 mmol) in 5 mL dry MeCN was added POCl₃ (0.0600 mL, 0.644 mmol). The reaction mixture was stirred for 1 hour under reflux. MeCN was evaporated off and the residue was treated with a solution of Intermediate 3 (0.0744 g, 0.264 mmol), DIPEA (0.22 mL, 1.26 mmol) in 5 mL dry MeCN. The reaction mixture was stirred for 1 hour and then was concentrated. The residue was first purified by gel silica column chromatography eluted with DCM/MeOH (100:0 to 90:10), then by reverse phase column chromatography (C-18, grain size: 40 μm, pore size distribution: 120 Å) eluted with CH₃CN/H₂O (10-80%) to give Example 24A (0.0012 g, fast eluted) and Example 24B (0.0018 g, slow eluted) as yellow solids. MS (ESI+): 628.3 [M+1]⁺.

### EXAMPLE 25

### (S)-2⁴-(4-acryloyl-2-methylpiperazin-1-yl)-2⁶,3⁶-difluoro-1⁶-isopropyl-2¹,2²-dihydro-4,7-diaza-2(1,7)-pyrido[2,3-d]pyrimidina-1,3(1,2)-dibenzenacycloheptaphane-2²,5-dione

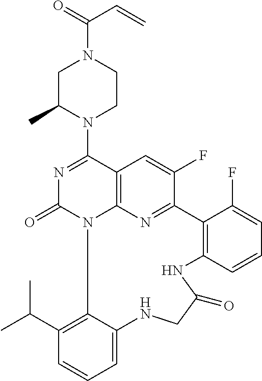 [SMILES: C=CC(=O)N1CCN(c2nc(=O)n3c4nc(c(F)cc24)-c2c(F)cccc2NC(=O)CNc2cccc(C(C)C)c2-3)[C@@H](C)C1]

### Step 1 tert-butyl N-(3-bromo-2-nitrophenyl)-N-(tert-butoxycarbonyl)glycinate

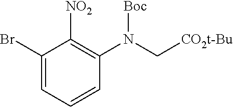 [SMILES: CC(C)(C)OC(=O)CN(C(=O)OC(C)(C)C)c1cccc(Br)c1[N+](=O)[O-]]

To a pre-cooled solution of 3-bromo-2-nitroaniline (5 g, 23.0 mmol) in dry THF (100 mL) was added NaH (2.03 g, 50.7 mmol) in portions in an ice bath under nitrogen atmosphere. After stirring at 0° C. for 0.5 h, Boc₂O (6.42 mL, 27.7 mmol) was added, stirred for another 2 h. BrCH₂CO₂^{t}Bu (4.07 mL, 27.7 mmol) was added, and the reaction mixture was stirred at rt overnight. The reaction mixture was poured into a mixture of water and sat. NH₄Cl (100/100 mL), extracted with ethyl acetate (80 mL×3). The combined extracts were concentrated and purified by silica gel column chromatography (petroleum ether/ethyl acetate: 100/0 to 95/5) to give the title compound (6.664 g) as a yellow oil. MS (ESI): 275.0/277.0 [M+H-isobutylene-Boc]⁺.

¹HNMR (400 MHz, CDCl₃) δ 7.74-7.66 (m, 1H), 7.62 (dd, J=8.2, 1.2 Hz, 1H), 7.42-7.32 (m, 1H), 4.52 (d, J=17.2 Hz, 1H), 3.60 (d, J=17.2 Hz, 1H), 1.51-1.43 (m, 13H), 1.34 (s, 6H).

### Step 2 tert-butyl N-(tert-butoxycarbonyl)-N-(2-nitro-3-(prop-1-en-2-yl)phenyl)glycinate

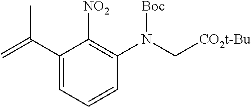 [SMILES: C=C(C)c1cccc(N(CC(=O)OC(C)(C)C)C(=O)OC(C)(C)C)c1[N+](=O)[O-]]

Starting with the product of Step 1 (6.66 g), the title product was obtained as a light-yellow oil (4.26 g) by following the procedure described in Step 3 of Example 21. MS (ESI): 237.1 [M+H-isobutylene-Boc]⁺.

¹HNMR (400 MHz, CDCl₃) δ 7.65-7.56 (m, 1H), 7.48-7.39 (m, 1H), 7.29-7.24 (m, 1H), 5.22-5.16 (m, 1H), 4.99 (s, 1H), 4.55 (d, J=17.7 Hz, 1H), 3.65 (d, J=17.7 Hz, 1H), 2.08 (s, 3H), 1.52-1.44 (m, 13H), 1.33 (s, 6H).

### Step 3 tert-butyl N-(2-amino-3-isopropylphenyl)-N-(tert-butoxycarbonyl)glycinate

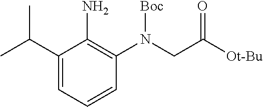 [SMILES: CC(C)c1cccc(N(CC(=O)OC(C)(C)C)C(=O)OC(C)(C)C)c1N]

To the solution of the product of Step 2 (4.45 g, 11.3 mmol) in MeOH (50 mL) were added 7M NH₃ in MeOH (0.5 mL) and 10% Pd/C (1.2 g, 1.13 mmol) under nitrogen atmosphere. The mixture was stirred under H₂ atmosphere at room temperature for 11.5 h. The solution was filtered through Celite, and the Celite pad was washed with EtOAc. The filtrate was concentrated to give the title compound (4.27 g) as a pale-yellow oil. MS (ESI): 265.2 [M+H]⁺.

### Step 4 tert-butyl N-(tert-butoxycarbonyl)-N-(2-(3-(2,6-dichloro-5-fluoronicotinoyl)ureido)-3-isopropylphenyl)glycinate

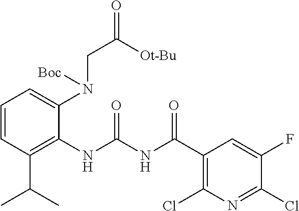 [SMILES: CC(C)c1cccc(N(CC(=O)OC(C)(C)C)C(=O)OC(C)(C)C)c1NC(=O)NC(=O)c1cc(F)c(Cl)nc1Cl]

Starting with 2,6-dichloro-5-fluoronicotinamide (2.34 g) and the product of Step 3 (3.31 g), the title product was obtained as a white solid (4.71 g) by following the procedure described in Step 3 of Example 1. MS (ESI): 597.0 [M−H]⁻.

### Step 5 tert-butyl N-(tert-butoxycarbonyl)-N-(2-(7-chloro-6-fluoro-2,4-dioxo-3,4-dihydropyrido[2,3-d]pyrimidin-1(2H)-yl)-3-isopropylphenyl)glycinate

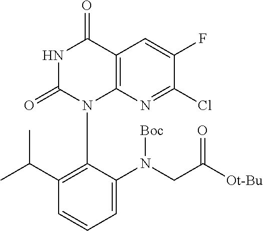 [SMILES: CC(C)c1cccc(N(CC(=O)OC(C)(C)C)C(=O)OC(C)(C)C)c1-n1c(=O)[nH]c(=O)c2cc(F)c(Cl)nc21]

Starting with the product of Step 4 (4.21 g), the title product was obtained as a white solid (2.73 g) by following the procedure described in Step 3 of Example 8. MS (ESI): 564.2 [M+H]⁺.

### Step 6 (2-(7-chloro-6-fluoro-2,4-dioxo-3,4-dihydropyrido[2,3-d]pyrimidin-1(2H)-yl)-3-isopropylphenyl)glycine

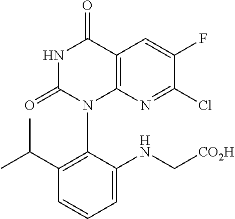 [SMILES: CC(C)c1cccc(NCC(=O)O)c1-n1c(=O)[nH]c(=O)c2cc(F)c(Cl)nc21]

To the solution of the product of Step 5 (600 mg, 1.07 mmol) in DCM (6 mL) was added TFA (4 mL). After stirring at rt for 16 h, 4 mL 1,4-dioxane was added and the solution was concentrated to give a brown oil, which was used for the next step without further purification. MS (ESI): 407.1 [M+H]⁺.

### Step 7 (2-(7-(2-amino-6-fluorophenyl)-6-fluoro-2,4-dioxo-3,4-dihydropyrido[2,3-d]pyrimidin-1(2H)-yl)-3-isopropylphenyl)glycine

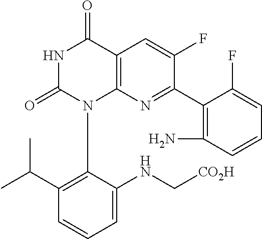 [SMILES: CC(C)c1cccc(NCC(=O)O)c1-n1c(=O)[nH]c(=O)c2cc(F)c(-c3c(N)cccc3F)nc21]

Starting with the product of Step 6 (505.5 mg) and 3-fluoro-2-(4,4,5,5-tetramethyl-1,3,2-dioxaborolan-2-yl)aniline (736.5 mg), the title product was obtained as a white solid (637 mg) by following the procedure described in Step 8 of Example 17. MS (ESI): 482.1 [M+H]⁺.

### Step 8 2⁶,3⁶-difluoro-1⁶-isopropyl-2¹,2²,2³,2⁴-tetrahydro-4,7-diaza-2(1,7)-pyrido[2,3-d]pyrimidina-1,3(1,2)-dibenzenacycloheptaphane-2²,2⁴,5-trione

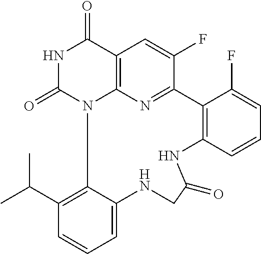 [SMILES: CC(C)c1cccc2c1-n1c(=O)[nH]c(=O)c3cc(F)c(nc31)-c1c(F)cccc1NC(=O)CN2]

To the turbid solution of the product of Step 7 (637 mg, 1.32 mmol) in dry MeCN (10 mL) was added pyridine (320 μL, 3.97 mmol). N-methylimidazole (320 μL, 3.97 mmol) was added, followed by dry DMF (10 mL) and TCFH (562.5 mg, 1.98 mmol). After stirring for 11 h, the reaction mixture was poured into 0.5N HCl solution (12 mL), and extracted with EtOAc (15 mL×4). The organic phase was washed with brine (5 mL×2), and the water phase was extracted with EtOAc (5 mL×2). The combined organic phase was concentrated and the residue was purified by silica gel column chromatography (petroleum ether/ethyl acetate: 100/0 to 50/50) to give the title product as a yellow solid (200 mg). MS (ESI): 464.1 [M+H]⁺.

### Step 9 (S)-2⁴-(4-acryloyl-2-methylpiperazin-1-yl)-2⁶,3⁶-difluoro-1⁶-isopropyl-2¹,2²-dihydro-4,7-diaza-2(1,7)-pyrido[2,3-d]pyrimidina-1,3(1,2)-dibenzenacycloheptaphane-2²,5-dione

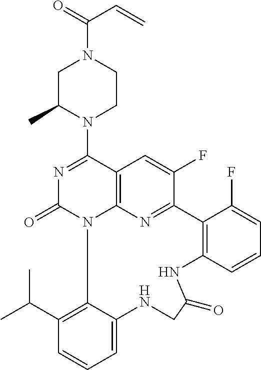 [SMILES: C=CC(=O)N1CCN(c2nc(=O)n3c4nc(c(F)cc24)-c2c(F)cccc2NC(=O)CNc2cccc(C(C)C)c2-3)[C@@H](C)C1]

Starting with the product of Step 8 (60 mg) and Intermediate 2 (69 mg), the title product was obtained as a white solid (2.14 mg) by following the procedure described in Step 13 of Example 17. MS (ESI): 600.3 [M+H]⁺.

### EXAMPLE 26

### 2⁴-((2S,5R)-4-acryloyl-2,5-dimethylpiperazin-1-yl)-2⁶,3⁶-difluoro-1⁶-isopropyl-2¹,2²-dihydro-4,7-diaza-2(1,7)-pyrido[2,3-d]pyrimidina-1,3(1,2)-dibenzenacycloheptaphane-2²,5-dione

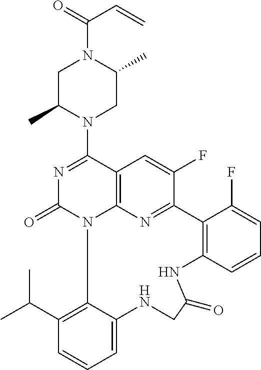 [SMILES: C=CC(=O)N1C[C@H](C)N(c2nc(=O)n3c4nc(c(F)cc24)-c2c(F)cccc2NC(=O)CNc2cccc(C(C)C)c2-3)C[C@H]1C]

Starting with the product of Step 8 (55 mg) and Intermediate 3 (133 mg), the title product was obtained as a yellow solid (7.8 mg) by following the procedure described in Step 13 of Example 17. MS (ESI): 614.3[M+H]⁺.

### EXAMPLE 27A and 27B

### 2⁴-((2S,5R)-4-acryloyl-2,5-dimethylpiperazin-1-yl)-2⁶,3⁶-difluoro-1⁶-isopropyl-2¹,2²-dihydro-4,7-diaza-2(1,7)-pyrido[2,3-d]pyrimidina-1,3(1,2)-dibenzenacycloheptaphan-2²-one

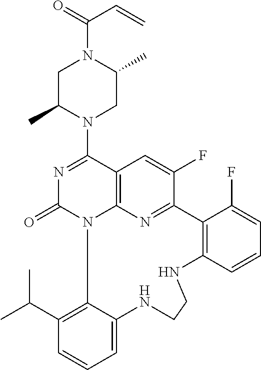 [SMILES: C=CC(=O)N1C[C@H](C)N(c2nc(=O)n3c4nc(c(F)cc24)-c2c(F)cccc2NCCNc2cccc(C(C)C)c2-3)C[C@H]1C]

### Step 1 2⁶,3⁶-difluoro-1⁶-isopropyl-2¹,2²,2³,2⁴-tetrahydro-4,7-diaza-2(1,7)-pyrido[2,3-d]pyrimidina-1,3(1,2)-dibenzenacycloheptaphane-2²,2⁴-dione

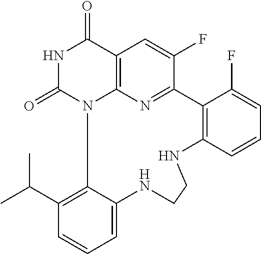 [SMILES: CC(C)c1cccc2c1-n1c(=O)[nH]c(=O)c3cc(F)c(nc31)-c1c(F)cccc1NCCN2]

Starting with the product of Step 8 of Example 25 (100 mg), the title product was obtained as a yellow solid (16 mg) by following the procedure described in Step 7 of Example 1. MS (ESI): 450.1 [M+H]⁺.

### Step 2 2⁴-((2S,5R)-4-acryloyl-2,5-dimethylpiperazin-1-yl)-2⁶,3⁶-difluoro-16-isopropyl-2¹,2²-dihydro-4,7-diaza-2(1,7)-pyrido[2,3-d]pyrimidina-1,3(1,2)-dibenzenacycloheptaphan-2²-one

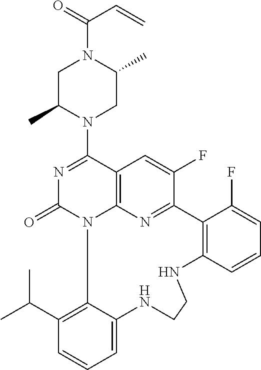 [SMILES: C=CC(=O)N1C[C@H](C)N(c2nc(=O)n3c4nc(c(F)cc24)-c2c(F)cccc2NCCNc2cccc(C(C)C)c2-3)C[C@H]1C]

To a solution of the product of Step 1 (20 mg, 0.04 mmol) in dry MeCN (2 mL) were added DIPEA (74 μL, 0.44 mmol) and POCl₃ (25 μL, 0.27 mmol) under argon atmosphere. After stirring at 80° C. for 1 h the reaction mixture was concentrated. To the residue were added DIEA (74 μL, 0.44 mmol) and a solution of 1-((2R,5S)-2,5-dimethylpiperazin-1-yl)prop-2-en-1-one (Intermediate 3, 75 mg, purity 30%, 0.13 mmol) in dry DMF (1.0 mL). After stirring at rt for 1.5 h, the reaction mixture was poured into water (5 mL), extracted with ethyl acetate (5 mL×4). The combined extracts were washed with sat. NH₄Cl (3 mL), concentrated and the residue was purified by silica gel column chromatography (petroleum ether/ethyl acetate: 80/20 to 0/100 then DCM/EtOAc: 90/10 to 20/80 then DCM/MeOH: 100/0 to 92/8) to provide Example 27A (fast eluted) as a yellow solid (5.4 mg) and a mixture of Example 27A and Example 27B (slow eluted). The mixture was further purified by prep-TLC (petroleum ether:ethyl acetate=1:5, eluted twice) to give another 1.0 mg of Example 27A and Example 27B as a yellow solid (5.6 mg). MS (ESI): 600.3[M+H]⁺.

Example 27A: ¹HNMR (400 MHz, CDCl₃) δ 7.83-7.73 (m, 1H), 7.31-7.26 (m, 1H), 7.26-7.19 (m, 1H), 6.81 (dd, J=17.4, 7.9 Hz, 2H), 6.66-6.54 (m, 1H), 6.54-6.45 (m, 2H), 6.44-6.34 (m, 1H), 5.84-5.75 (m, 1H), 5.38-5.27 (m, 1H), 5.14 (brs, 1H), 5.04-4.94 (m, 1H), 4.40-4.27 (m, 1H), 4.14-4.06 (m, 1H), 4.04-3.97 (m, 1H), 3.97-3.89 (m, 1H), 3.84 (d, J=12.6 Hz, 1H), 3.71-3.61 (m, 1H), 3.33-3.18 (m, 2H), 3.18-3.08 (m, 1H), 2.70-2.58 (m, 1H), 1.39 (d, J=6.8 Hz, 2H), 1.37-1.24 (m, 4H), 1.21 (d, J=6.8 Hz, 2H), 1.16 (d, J=6.8 Hz, 2H), 0.84 (d, J=6.8 Hz, 2H).

Example 27B: ¹HNMR (400 MHz, CDCl₃) δ 7.82 (dd, J=19.6, 9.2 Hz, 1H), 7.30-7.27 (m, 1H), 7.25-7.19 (m, 1H), 6.88-6.74 (m, 2H), 6.67 (dd, J=16.9, 10.9 Hz, 1H), 6.59-6.34 (m, 3H), 5.87-5.75 (m, 1H), 5.21-5.12 (m, 1H), 4.98 (d, J=14.5 Hz, 1H), 4.90-4.71 (m, 1H), 4.61-4.48 (m, 1H), 4.45-4.36 (m, 1H), 3.92-3.81 (m, 1H), 3.79-3.53 (m, 3H), 3.33-3.06 (m, 3H), 2.67-2.56 (m, 1H), 1.64 (d, J=6.8 Hz, 1H), 1.56-1.41 (m, 7H), 1.20 (d, J=6.8 Hz, 2H), 0.99-0.89 (m, 2H).

### EXAMPLE 28

### (S)-2⁴-(4-acryloyl-2-methylpiperazin-1-yl)-2⁶,3⁶-difluoro-1⁶-isopropyl-8-methyl-2¹,2²-dihydro-4,8-diaza-2(1,7)-pyrido[2,3-d]pyrimidina-1,3(1,2)-dibenzenacyclooctaphan-2²-one

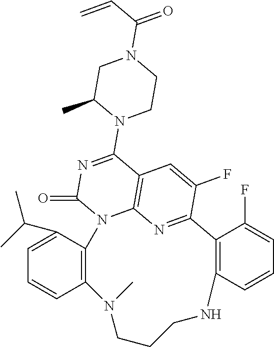 [SMILES: C=CC(=O)N1CCN(c2nc(=O)n3c4nc(c(F)cc24)-c2c(F)cccc2NCCCN(C)c2cccc(C(C)C)c2-3)[C@@H](C)C1]

### Step 1 ethyl 3-((3-bromo-2-nitrophenyl)(methyl)amino)propanoate

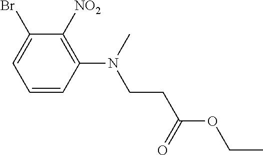 [SMILES: CCOC(=O)CCN(C)c1cccc(Br)c1[N+](=O)[O-]]

Starting with ethyl 3-(methylamino)propanoate and 1-bromo-3-fluoro-2-nitrobenzene, the title product was obtained as a yellow oil by following the procedure described in Step 1 of Example 21. MS: (ESI+): 353.7 [M+23]⁺.

### Step 2 ethyl 3-(methyl(2-nitro-3-(prop-1-en-2-yl)phenyl)amino)propanoate

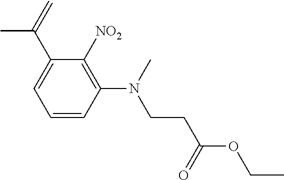 [SMILES: C=C(C)c1cccc(N(C)CCC(=O)OCC)c1[N+](=O)[O-]]

Starting with the product of Step 1 and isopropenylboronic acid pinacol ester, the title product was obtained as a yellow oil by following the procedure described in Step 3 of Example 21. MS: (ESI+): 293.1 [M+1]⁺.

### Step 3 ethyl 3-((2-amino-3-isopropylphenyl)(methyl)amino)propanoate

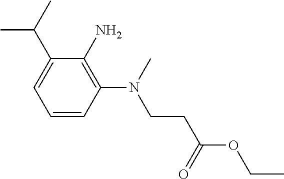 [SMILES: CCOC(=O)CCN(C)c1cccc(C(C)C)c1N]

Starting with the product of Step 2, the title product was obtained as an oil by following the procedure described in Step 4 of Example 21. MS: (ESI+): 265.1 [M+1]⁺.

### Step 4 ethyl 3-((2-(3-(2,6-dichloro-5-fluoronicotinoyl)ureido)-3-isopropylphenyl)(methyl)amino)propanoate

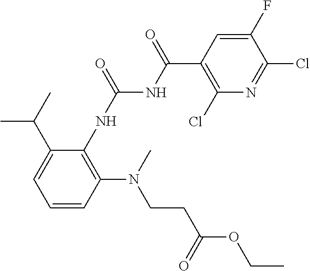 [SMILES: CCOC(=O)CCN(C)c1cccc(C(C)C)c1NC(=O)NC(=O)c1cc(F)c(Cl)nc1Cl]

Starting with the product of Step 3, the title product was obtained as a yellow solid by following the procedure described in Step 5 of Example 21. MS: (ESI+): 499.1 [M+1]⁺.

### Step 5 ethyl 3-((2-(7-chloro-6-fluoro-2,4-dioxo-3,4-dihydropyrido[2,3-d]pyrimidin-1(2H)-yl)-3-isopropylphenyl)(methyl)amino)propanoate

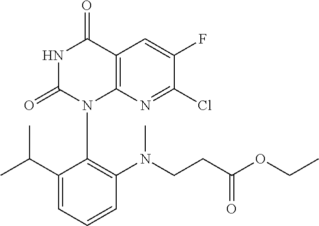 [SMILES: CCOC(=O)CCN(C)c1cccc(C(C)C)c1-n1c(=O)[nH]c(=O)c2cc(F)c(Cl)nc21]

Starting with 2,6-dichloro-5-fluoronicotinamide (2.45 g), the title product was obtained as a brown solid (1.19 g) by following the procedure described in Step 3 of Example 8. MS: (ESI+): 463.2 [M+1]⁺.

### Step 6 ethyl 3-((2-(7-(2-amino-6-fluorophenyl)-6-fluoro-2,4-dioxo-3,4-dihydropyrido[2,3-d]pyrimidin-1(2H)-yl)-3-isopropylphenyl)(methyl)amino)propanoate

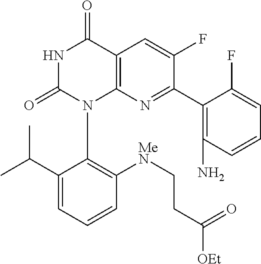 [SMILES: CCOC(=O)CCN(C)c1cccc(C(C)C)c1-n1c(=O)[nH]c(=O)c2cc(F)c(-c3c(N)cccc3F)nc21]

Starting with the product of Step 5 (0.67 g) and 3-fluoro-2-(4,4,5,5-tetramethyl-1,3,2-dioxaborolan-2-yl)aniline (0.75 g), the title product was obtained as a brown solid (0.53 g) by following the procedure described in Step 8 of Example 17. MS: (ESI+): 538.2 [M+1]⁺.

### Step 7 3-((2-(7-(2-amino-6-fluorophenyl)-6-fluoro-2,4-dioxo-3,4-dihydropyrido[2,3-d]pyrimidin-1(2H)-yl)-3-isopropylphenyl)(methyl)amino)propanoic acid

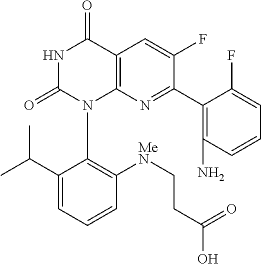 [SMILES: CC(C)c1cccc(N(C)CCC(=O)O)c1-n1c(=O)[nH]c(=O)c2cc(F)c(-c3c(N)cccc3F)nc21]

To a solution of the product of Step 6 (420 mg) in THF (2.5 mL), was added LiOH (141 mg) in H₂O (2.5 mL). After stirring at rt for 2 hours, the reaction mixture was concentrated to dryness and dissolved in water (10 mL), washed with Et₂O (2 mL×5). The water phase was acidified with 1N HCl to pH=7 and concentrated under reduced pressure to dryness. The residue was co-evaporated with toluene (2 mL×3) to obtain the title compound as lithium salt (400 mg, yellow solid). MS: (ESI+): 510.2 [M+1]⁺.

### Step 8 2⁶,3⁶-difluoro-1⁶-isopropyl-8-methyl-2¹,2²,2³,2⁴-tetrahydro-4,8-diaza-2(1,7)-pyrido[2,3-d]pyrimidina-1,3(1,2)-dibenzenacyclooctaphane-2²,2⁴,5-trione

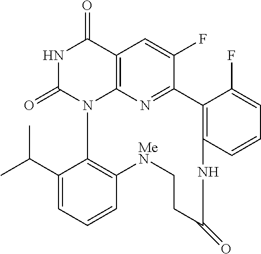 [SMILES: CC(C)c1cccc2c1-n1c(=O)[nH]c(=O)c3cc(F)c(nc31)-c1c(F)cccc1NC(=O)CCN2C]

Starting with the product of Step 7 (0.20 g), the title product was obtained as a yellow solid (0.14 g) by following the procedure described in Step 7 of Example 1. MS: (ESI+): 492.2 [M+1]⁺.

### Step 9 2⁶,3⁶-difluoro-1⁶-isopropyl-8-methyl-2¹,2²,2³,2⁴-tetrahydro-4,8-diaza-2(1,7)-pyrido[2,3-d]pyrimidina-1,3(1,2)-dibenzenacyclooctaphane-2²,2⁴-dione

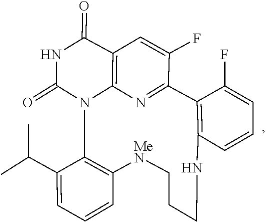 [SMILES: CC(C)c1cccc2c1-n1c(=O)[nH]c(=O)c3cc(F)c(nc31)-c1c(F)cccc1NCCCN2C]

Starting with the product of Step 8 (10 mg), the title product was obtained as a yellow solid (13 mg, crude) by following the procedure described in Step 1 of Example 2. MS: (ESI+): 478.1 [M+1]⁺.

### Step 10 (S)-2⁴-(4-acryloyl-2-methylpiperazin-1-yl)-2⁶,3⁶-difluoro-1⁶-isopropyl-8-methyl-2¹,2²-dihydro-4,8-diaza-2(1,7)-pyrido[2,3-d]pyrimidina-1,3(1,2)-dibenzenacyclooctaphan-2²-one

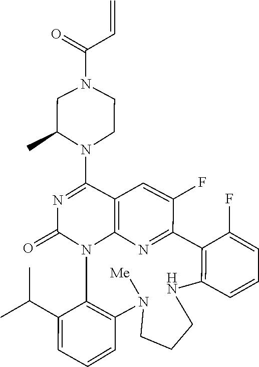 [SMILES: C=CC(=O)N1CCN(c2nc(=O)n3c4nc(c(F)cc24)-c2c(F)cccc2NCCCN(C)c2cccc(C(C)C)c2-3)[C@@H](C)C1]

Starting with the product of Step 9 (10 mg) and Intermediate 2 (47 mg), the title product was obtained as a brown solid (3.23 mg) by following the procedure described in Step 13 of Example 17. MS (ESI+): 614.3 [M+1]⁺.

### EXAMPLE 29

### (S)-2⁴-(4-acryloyl-2-methylpiperazin-1-yl)-2⁶,3⁶-difluoro-1⁶-isopropyl-8-methyl-2¹,2²-dihydro-4,8-diaza-2(1,7)-pyrido[2,3-d]pyrimidina-1,3(1,2)-dibenzenacyclooctaphane-2²,5-dione

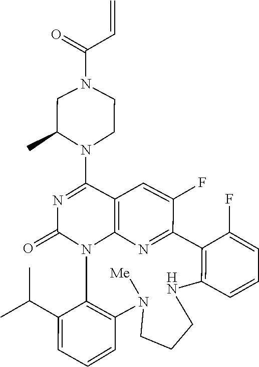 [SMILES: C=CC(=O)N1CCN(c2nc(=O)n3c4nc(c(F)cc24)-c2c(F)cccc2NCCCN(C)c2cccc(C(C)C)c2-3)[C@@H](C)C1]

Starting with the product of Step 8 (15 mg) of Example 28 and Intermediate 2 (30 mg), the title product was obtained as a yellow solid (0.85 mg) by following the procedure described in Step 13 of Example 17. MS (ESI+): 628.3 [M+1]⁺.

### EXAMPLE 30

### 2⁴-((2S,5R)-4-acryloyl-2,5-dimethylpiperazin-1-yl)-2⁶,3⁶-difluoro-1⁶-isopropyl-8-methyl-2¹,2²-dihydro-4,8-diaza-2(1,7)-pyrido[2,3-d]pyrimidina-1,3(1,2)-dibenzenacyclooctaphane-2²,5-dione

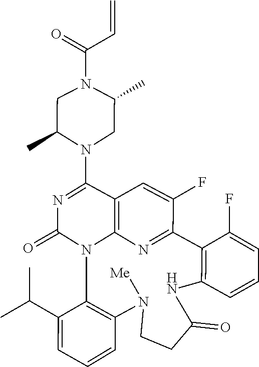 [SMILES: C=CC(=O)N1C[C@H](C)N(c2nc(=O)n3c4nc(c(F)cc24)-c2c(F)cccc2NC(=O)CCN(C)c2cccc(C(C)C)c2-3)C[C@H]1C]

Starting with the product of Step 8 (40 mg) of Example 28 and Intermediate 3 (245 mg), the title product was obtained as a white solid (12 mg) by following the procedure described in Step 13 of Example 17, except the crude was purified by C-18 reverse phase column chromatography eluted with a gradient of aqueous 0.1% formic acid solution/MeCN (100% to 0%) to obtain the title compound (2.9 mg; Yield: 5.68%) as a white solid. MS (ESI+): 642.3 [M+1]⁺.

### EXAMPLE 31

### (S)-2⁴-(4-acryloyl-2-methylpiperazin-1-yl)-2⁶,3⁶-difluoro-1²-isopropyl-4-methyl-2¹,2²-dihydro-7-thia-4-aza-2(1,7)-pyrido[2,3-d]pyrimidina-1(3,4)-pyridina-3(1,2)-benzenacycloheptaphan-2²-one

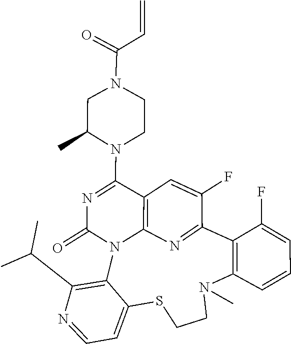 [SMILES: C=CC(=O)N1CCN(c2nc(=O)n3c4nc(c(F)cc24)-c2c(F)cccc2N(C)CCSc2ccnc(C(C)C)c2-3)[C@@H](C)C1]

### Step 1 tert-butyl 2-mercaptoacetate

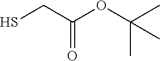

The mixture of tert-butyl 2-bromoacetate (20 g, 0.10 mol) and potassium ethanethioate (18 g, 0.16 mol) in EtOH (200 ml) was stirred for 16 h at rt. The reaction mixture was treated with 2N NaOH (205 ml) and stirred for 1 h. The pH of the mixture was adjusted with 1N HCl to 7 and the reaction mixture was extracted with EA. The organic layer was washed with brine, dried over anhydrous Na₂SO₄, filtered and concentrated to get 8.5 g of crude product as brown oil which was used to the next step without further purification.

### Step 2 tert-butyl 2-((3-amino-2-isopropylpyridin-4-yl)thio)acetate

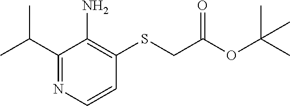 [SMILES: CC(C)c1nccc(SCC(=O)OC(C)(C)C)c1N]

The mixture of Intermediate 5 (6.25 g, 23.86 mmol), tert-butyl 2-mercaptoacetate (8.84 g, 59.64 mmol), DIPEA (12.31 g, 95.42 mmol), Pd₂(dba)₃ (4.37 g, 4.77 mmol) and Xantphos (5.52 g, 9.54 mmol) in 1,4-Dioxane (30 ml) was heat to 90° C. and stirred for 2 h under Ar. After the reaction was completed, the mixture was cool to rt, quenched with water and extracted with EA. The organic layer was washed with brine, dried over anhydrous Na₂SO₄, filtered and concentrated. The residue was purified by silica gel column chromatography (PE:EA=1:1) to give 4.6 g of pure product as a yellow solid. MS (ES+): 283 [M+1]⁺

### Step 3 tert-butyl 2-((3-(3-(2,6-dichloro-5-fluoronicotinoyl)ureido)-2-isopropylpyridin-4-yl)thio)acetate

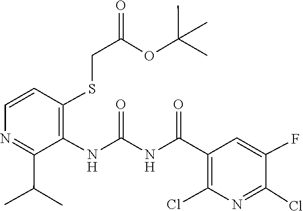 [SMILES: CC(C)c1nccc(SCC(=O)OC(C)(C)C)c1NC(=O)NC(=O)c1cc(F)c(Cl)nc1Cl]

Starting with 2,6-dichloro-5-fluoronicotinamide (6.67 g) and the product of Step 2 (4.5 g), the title product was obtained as a yellow solid (3.1 g) by following the procedure described in Step 3 of Example 1. MS (ES+): 517 [M+1]⁺.

### Step 4 tert-butyl 2-((3-(7-chloro-6-fluoro-4-hydroxy-2-oxopyrido[2,3-d]pyrimidin-1(2H)-yl)-2-isopropylpyridin-4-yl)thio)acetate

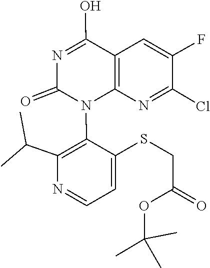 [SMILES: CC(C)c1nccc(SCC(=O)OC(C)(C)C)c1-n1c(=O)nc(O)c2cc(F)c(Cl)nc21]

Starting with the product of Step 3 (3.0 g), the title product was obtained as a white solid (2.1 g) by following the procedure described in Step 3 of Example 8. MS (ES+): 481 [M+1]⁺.

### Step 5 tert-butyl 2-((3-(7-(2-amino-6-fluorophenyl)-6-fluoro-4-hydroxy-2-oxopyrido[2,3-d]pyrimidin-1(2H)-yl)-2-isopropylpyridin-4-yl)thio)acetate

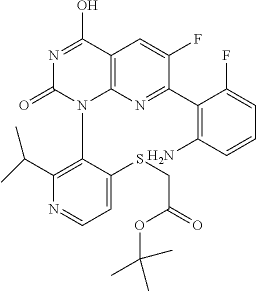 [SMILES: CC(C)c1nccc(SCC(=O)OC(C)(C)C)c1-n1c(=O)nc(O)c2cc(F)c(-c3c(N)cccc3F)nc21]

Starting with the product of Step 4 (700 mg) and 3-fluoro-2-(4,4,5,5-tetramethyl-1,3,2-dioxaborolan-2-yl)aniline (3.45 g), the title product was obtained as a white solid (575 mg) by following the procedure described in Step 8 of Example 17. MS (ES+): 556 [M+1]⁺.

### Step 6 2⁶,3⁶-difluoro-2⁴-hydroxy-1²-isopropyl-2¹,2²-dihydro-7-thia-4-aza-2(1,7)-pyrido[2,3-d]pyrimidina-1(3,4)-pyridina-3(1,2)-benzenacycloheptaphane-2²,5-dione

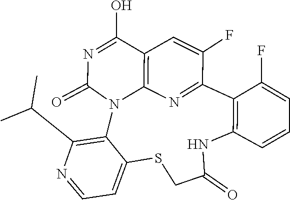 [SMILES: CC(C)c1nccc2c1-n1c(=O)nc(O)c3cc(F)c(nc31)-c1c(F)cccc1NC(=O)CS2]

To a stirred solution of the product of Step 5 (240 mg, 0.43 mmol) in DCM (3 ml) was added TFA (3 ml) at rt. The resulting mixture was stirred for 1 h at rt, and then concentrated. The residue was dissolved in DMF (60 ml). NMI (532 mg, 6.49 mmol) and TCFH (393 mg, 1.30 mmol) were added at rt under Ar. The resulting mixture was stirred for 1 h at rt and then quenched with water and extracted with EA. The organic layer was washed with brine, dried over anhydrous Na₂SO₄, filtered and concentrated. The residue was purified by Prep-TLC (PE:EA=1:1) to get 92 mg of the title product as a yellow solid. MS (ES+): 482 [M+1]⁺.

### Step 7 2⁶,3⁶-difluoro-2⁴-hydroxy-12-isopropyl-2¹,2²-dihydro-7-thia-4-aza-2(1,7)-pyrido[2,3-d]pyrimidina-1(3,4)-pyridina-3(1,2)-benzenacycloheptaphan-2²-one

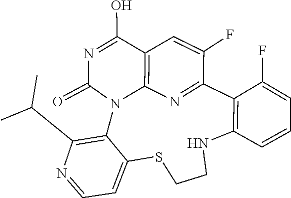 [SMILES: CC(C)c1nccc2c1-n1c(=O)nc(O)c3cc(F)c(nc31)-c1c(F)cccc1NCCS2]

To a stirred solution of the product of Step 6 (92 mg, 0.19 mmol) in THF (1 ml) was added borane-tetrahydrofuran complex (1 M, 0.6 ml, 0.57 mmol) at 0° C. under Ar balloon. The mixture was stirred for 1 h at rt under Ar and then quenched with water and extracted with EA. The organic layer was washed with brine, dried over anhydrous Na₂SO₄, filtered and concentrated. The residue was purified by Prep-TLC (PE:EA=2:1) to get 57 mg of the title product as a yellow solid. MS (ES+): 468 [M+1]⁺.

### Step 8 (S)-2⁴-(4-acryloyl-2-methylpiperazin-1-yl)-2⁶,3⁶-difluoro-12-isopropyl-2¹,2²-dihydro-7-thia-4-aza-2(1,7)-pyrido[2,3-d]pyrimidina-1(3,4)-pyridina-3(1,2)-benzenacycloheptaphan-2²-one

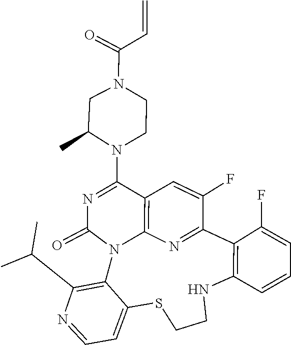 [SMILES: C=CC(=O)N1CCN(c2nc(=O)n3c4nc(c(F)cc24)-c2c(F)cccc2NCCSc2ccnc(C(C)C)c2-3)[C@@H](C)C1]

Starting with the product of Step 7 (57 mg) and Intermediate 2 (79 mg), the title product was obtained as a yellow solid (37 mg) by following the procedure described in Step 8 of Example 17. MS (ES+): 604 [M+1]⁺.

### Step 9 (S)-2⁴-(4-acryloyl-2-methylpiperazin-1-yl)-2⁶,3⁶-difluoro-12-isopropyl-4-methyl-2¹,2²-dihydro-7-thia-4-aza-2(1,7)-pyrido[2,3-d]pyrimidina-1(3,4)-pyridina-3(1,2)-benzenacycloheptaphan-2²-one

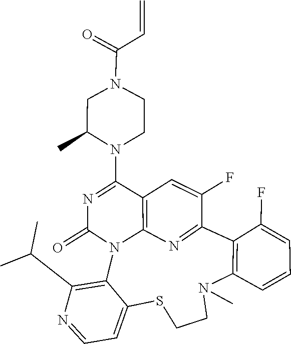 [SMILES: C=CC(=O)N1CCN(c2nc(=O)n3c4nc(c(F)cc24)-c2c(F)cccc2N(C)CCSc2ccnc(C(C)C)c2-3)[C@@H](C)C1]

To a stirred solution of the product of Step 8 (35 mg, 0.06 mmol) in THF (1 ml) were added HCHO (30%) (5 ml) and STAB (121 mg, 0.60 mmol) at rt. After stirring for 1 h, the mixture was quenched with water and extracted with EA. The organic layer was washed with brine, dried over anhydrous Na₂SO₄, filtered and concentrated. The residue was purified by Prep-TLC (EA) to get 17 mg of the title product as a yellow solid. MS (ES+): 618 [M+1]⁺.

### Synthesis of Intermediates

### Intermediate 1

### (S)-1-(3-methylpiperazin-1-yl)prop-2-en-1-one 2,2,2-trifluoroacetate

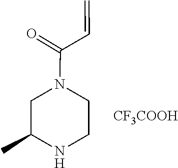

### Step 1 tert-butyl (S)-4-acryloyl-2-methylpiperazine-1-carboxylate

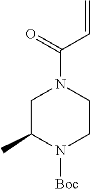 [SMILES: C=CC(=O)N1CCN(C(=O)OC(C)(C)C)[C@@H](C)C1]

To a stirred solution of tert-butyl (S)-2-methylpiperazine-1-carboxylate (4.7971 g, 23.952 mmol) and triethylamine (8.0 mL, 57.4 mmol) in DCM (80 mL) was added acrylyl chloride (2.10 mL, 25.8 mmol) in 15 minutes at 0° C. The reaction mixture was then treated with 20 mL of water and extracted with 200 mL of EtOAc. The organic layer was washed with 60 mL of 2 N HCl (aq), and 5×50 mL water, brine and dried over Na₂SO₄. The solution was filtered, concentrated to give the title compound as a light yellow oil. MS (ESI+): 277.1 [M+23]⁺.

### Step 2 (S)-1-(3-methylpiperazin-1-yl)prop-2-en-1-one 2,2,2-trifluoroacetate

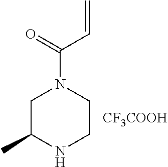

A solution of tert-butyl (S)-4-acryloyl-2-methylpiperazine-1-carboxylate (3.57 g, 14.0 mmol) and TFA (8.0 mL, 107.7 mmol) in DCM (20 mL) was stirred for 5 h at rt. Then 20 mL toluene was added. The resulted mixture was evaporated off to give the title compound (4.24 g) as a light oil. MS (ESI+): 155.1[M+1]⁺.

### Intermediate 2

### (S)-1-(3-methylpiperazin-1-yl)prop-2-en-1-one

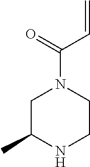

The crude Intermediate 1 (4.24 g) was dissolved in 25 mL NaOH (aq) (2.67 g NaOH). The reaction mixture was saturated with 10 g NaCl and extracted with 3×30 mL of DCM. The combined organic layer was dried over Na₂SO₄ and concentrated to give the Intermediate 2 (2.3 g). MS (ESI+): 155.1[M+1]⁺.

### Intermediate 3

### 1-((2R,5S)-2,5-dimethylpiperazin-1-yl)prop-2-en-1-one 2,2,2-trifluoroacetate

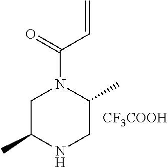

### Step 1 tert-butyl (2S,5R)-4-acryloyl-2,5-dimethylpiperazine-1-carboxylate

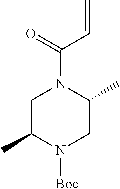

To a stirred solution of tert-butyl (2S,5R)-2,5-dimethylpiperazine-1-carboxylate (3.83 g, 17.9 mmol) and triethylamine (5.2 mL, 37.3 mmol) in DCM (30 mL) was added acrylylchloride (1.70 mL, 20.9 mmol) at 0° C. After stirring for 15 min at 0° C., water (10 mL) was added and the reaction mixture was stirred for additional 1 h at rt. The mixture was extracted with 50 mL EtOAc and the organic layer was washed with 20 mL 2 N HCl (aq), 2×20 mL 0.2 M HCl (aq), 2×20 mL water, brine and dried over Na₂SO₄. The solution was filtered and concentrated to give the title compound as a light oil. MS (ESI+): 291.1 [M+23]⁺.

### Step 2 1-((2R,5S)-2,5-dimethylpiperazin-1-yl)prop-2-en-1-one 2,2,2-trifluoroacetate

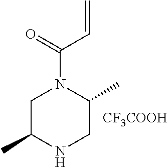

A solution of the product of Step 1 (3.85 g, 14.3 mmol) and TFA (10.0 mL, 134.6 mmol) in DCM (40 mL) was stirred for 1 h at rt. Toluene (40 mL) was added and the reaction mixture was evaporated off to give the title compound (4.0 g) as a light yellow oil. MS (ESI+): 169.2 [M+1]⁺.

### Intermediate 4

### 3-chloro-1-((2R,5S)-2,5-dimethylpiperazin-1-yl)propan-1-one hydrochloride

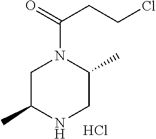

The product of Step 1 of Intermediate 3 (200.0 mg, 0.746 mmol) was added to 4N HCl/dioxane solution (1.0 mL). After stirring at rt for 1 h, the reaction mixture was concentrated to dryness to afford the title compound (150 mg). MS (ESI+): 205.1 [M+1]⁺.

1HNMR (400 MHz, DMSO-d₆): δ 3.82-3.79 (m, 2H), 0.60-3.57 (m, 4H), 3.45-3.35 (m, 2H), 2.99-2.95 (m, 1H), 2.82-2.75 (m, 1H), 1.29-1.21 (m, 6H).

### Intermediate 5

### 2-iodo-4-isopropylpyridin-3-amine

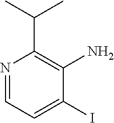

### Step 1 2,4-dichloropyridin-3-amine

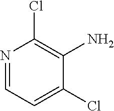

To a stirred solution of 2,4-dichloro-3-nitropyridine (150 g, 0.78 mol) in CH₃COOH (750 ml) was added Fe (140 g, 2.49 mol) at rt. After stirring for 3 h at 40° C., the pH of the reaction mixture was adjusted to 8˜9 by Na₂CO₃ (aq.) and extracted with EA. The organic layer was washed with brine, dried over anhydrous Na₂SO₄, filtered and concentrated to give 120 g of the crude title product as a brown solid. MS (ES+): 162.9 [M+1]⁺.

### Step 2 4-chloro-2-(prop-1-en-2-yl)pyridin-3-amine

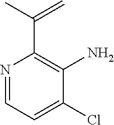

To a stirred solution of 2,4-dichloropyridin-3-amine (85 g, 0.52 mol), 1-(4,4,5,5-tetramethyl-1,3,2-dioxaborolan-2-yl)ethan-1-one (115 g, 0.68 mol), K₃PO₄ (287 g, 1.36 mol) in THF (1.7 L)/H₂O (340 ml) was added Pd(dppf)Cl₂.DCM (32 g, 0.039 mol) at rt under Ar. The mixture was stirred for 7 h at 75° C. and then quenched with water and extracted with EA. The organic layer was washed with brine, dried over anhydrous Na₂SO₄, filtered and concentrated. The residue was purified by silica gel column chromatography (PE:EA=2:1) to give 84 g of the title product as a white solid. MS (ES+): 168.9 [M+1]⁺.

### Step 3 4-iodo-2-isopropylpyridin-3-amine

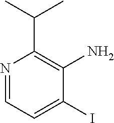

The mixture of 4-chloro-2-(prop-1-en-2-yl)pyridin-3-amine (84 g, 0.5 mol) in HI (aq. 55-58%, 1.26 L) was stirred overnight at 120° C. under Ar. The pH of the reaction mixture was then adjusted to 9-10 with Na₂CO₃ (aq.) and extracted with EA. The organic layer was washed with NaHSO₃ (aq.) and brine, dried over anhydrous Na₂SO₄, filtered and concentrated to give 90 g of crude product as brown oil which was used to the next step without further purification. MS (ES+): 262.8 [M+1]⁺.

All literatures mentioned in the present application are incorporated herein by reference, as though each one is individually incorporated by reference. Additionally, it should be understood that after reading the above teachings, those skilled in the art can make various changes and modifications to the present invention. These equivalents also fall within the scope defined by the appended claims.

1. A compound of Formula VIIA or a pharmaceutically acceptable salt thereof: 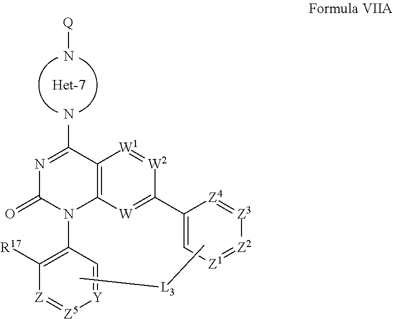
where 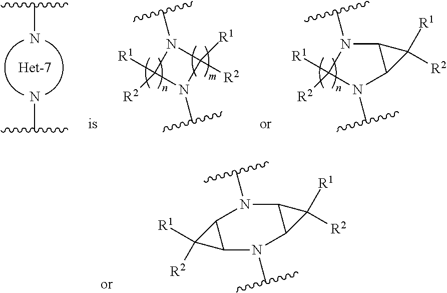
R¹ and R² are independently selected from the group consisting of hydrogen, halo, C₀₋₆alkylene-CN, C₀₋₆alkyleneNR¹⁹R²⁰, C₁₋₆alkoxy, hydroxy, C₀₋₆alkylene-C(O)NH₂, C₀₋₆alkylene-C(O)NHC₁₋₆ alkyl, C₀₋₆alkylene-C(O)N(C₁₋₆ alkyl)₂, C₀₋₆alkylene-S(O)₂—C₁₋₆alkyl, C₀₋₆alkylene-S(O)₂NH₂, C₀₋₆alkylene-S(O)₂NHC₁₋₆alkyl, C₀₋₆alkylene-S(O)₂N(C₁₋₆ alkyl)₂, C₀₋₆alkylene-NHC(O)NH₂, C₀₋₆alkylene-NHC(O)NHC₁₋₆alkyl, C₀₋₆ alkylene-NR¹⁹C(O)N(C₁₋₆ alkyl)₂, C₁₋₆alkyl, C₀₋₆ alkylene-NHC(O)OC₁₋₆alkyl, C₀₋₆ alkylene-C(O)—C₁₋₆ alkyl, C₁₋₆heteroalkyl, C₀₋₆ alkylene-heterocyclyl, and C₀₋₆ alkylene-heterocyclylalkyl; or R¹ and R², together with the carbon atom to which they are attached, can form a 3 to 6 membered carbocyclic ring;
Z and Y are each independently N or CR³;
W is N or CR⁶;
W¹ is N or CR³;
W² is N or CR⁴;
Z¹, Z², Z³, Z⁴ and Z⁵ are each independently N or CR¹⁸;
R³, R⁴ and R⁶ are each independently selected from the group consisting of H, OH, CN or halo, C₁₋₆alkyl, C₃₋₁₀cycloalkyl, C₃₋₁₀heteroalkyl, C₃₋₁₀heterocylcoalkyl, C₁₋₆haloalkyl, C₁₋₆alkoxy, NH—C₁₋₆alkyl, N(C₁₋₆alkyl)₂, C₃₋₈cycloalkyl, C₂₋₄alkenyl, C₂₋₄alkynyl, C₂₋₆heterocyclyl, aryl and heteroaryl;
R¹⁷ and R¹⁸ are each independently selected from the group consisting of halogen, CN, a branched or a linear C₁₋₆ alkyl, C₃₋₆ cycloalkyl, C₃₋₆ heterocycyl, —SC₁₋₆ alkyl, —OC₁₋₆ alkyl. —OC₃₋₆ heterocycyl, —OC₃₋₆ cycyl, NH—C₁₋₆ alkyl, N(C₁₋₆ alkyl)₂, —SC₃₋₆ heterocycyl, —SC₃₋₆ cycyl, —S(O)C₁₋₆ alkyl, —S(O)₂C₁₋₆ alkyl, —S(O)₂NH₂, —S(O)₂NHC₁_₆ alkyl, —S(O)₂N(C₁₋₆ alkyl)₂, —P(O)(C₁₋₆ alkyl)₂, C₂₋₆ heterocyclyl, an C₆₋₁₀ aryl and a C₁₋₈heteroaryl;
L₃ is selected from the group consisting of —(CH₂)_{q}, —(CH₂)_{q}C(O)—, —O(CH₂)_{q}C(O)—, —NR¹⁹(CH₂)_{q}NR²⁰—, —(CH₂)_{q}NR²⁰—, —O(CH₂)_{q}O—, —(CH₂)_{q}C(O)NR¹⁹—, —(CH₂)_{q}C(S)NR¹⁹—, —(CH₂)_{q}CHCF₃NR¹⁹—, —(CH₂)_{q}NR¹⁹C(O)—, —(CH₂)_{q}NR¹⁹CHCF₃—, —C(O)NR¹⁹(CH₂)_{q}—, —CHCF₃NR¹⁹(CH₂)_{q}—, —C(S)NR¹⁹(CH₂)_{q}—, —O(CH₂)_{q}C(O)NR¹⁹—, —O(CH₂)_{q}C(S)NR¹⁹—, —S(O)ᵥ(CH₂)_{q}C(O)—, —O(CH₂)_{q}C(O)NR¹⁹—, —NR¹⁹C(O)(CH₂)_{q}C(O)NR²⁰, —C(O)NR¹⁹(CH₂)_{q}C(O)NR²⁰—, —C(O)NR¹⁹(CH₂)_{q}NR²⁰C(O)—, —NR¹⁹C(O)(CH₂)_{q}NR²⁰C(O)—, O(CH₂)_{q}CNR¹⁹—, —S(O)ᵥ(CH₂)_{q}O—, —O(CH₂)_{q}S(O)ᵥ—, —S(O)ᵥ(CH₂)_{q}—, —(CH₂)_{q}S(O)ᵥ—, —S(O)ᵥ(CH₂)_{q}S(O)ᵥ—, —NR¹⁹(CH₂)_{q}C(O)NR²⁰—, —NR¹⁹(CH₂)_{q}—, —NR¹⁹C(O)(CH₂)_{q}—, —NR¹⁹CHCF₃(CH₂)_{q}—, —NR¹⁹(CH₂)_{q}O—, —(CH₂)ᵣOC(O)(CH₂)_{q}—, —OC(O)(CH₂)_{q}—, —OC(O)(CH₂)_{q}S(O)ᵥ—, —NR¹⁹(CH₂)_{q}CH═CH(CH₂)ᵣ—, NR¹⁹C(O)(CH₂)_{q}CH═CH(CH₂)ᵣ—, —(CH₂)_{q}CH═CH(CH₂)ᵣC(O)NR²⁰—, —(CH₂)_{q}NR¹⁹C(O)NR²⁰(CH₂)ᵣ—, —(CH₂)_{q}NR¹⁹C(S)NR²⁰(CH₂)ᵣ—, —(CH₂)_{q}NR¹⁹S(O)₂NR²⁰(CH₂)ᵣ—, —(CH₂)_{q}S(O)ᵥ(CH₂)ᵣ—, —(CH₂)_{q}S(O)₂NR²⁰(CH₂)ᵣ—, —(CH₂)_{q}NR¹⁹S(O)ᵥ(CH₂)ᵣ—, —(CH₂)_{q}SS(CH₂)ᵣ—, —(CH₂)_{q}S(CH₂)ᵣ—, —(CH₂)_{q}O(CH₂)ᵣ—, —(CH₂)_{q}NR¹⁹(CH₂)ᵣ—, —(CH₂)_{q}C≡C(CH₂)ᵣ—, —O(CH₂)_{q}CH═CH(CH₂)ᵣ—, —O(CH₂)_{q}CH≡CH(CH₂)ᵣ—, —(CH₂)_{q}CH≡CH(CH₂)ᵣO—, —O(CH₂)_{q}CH═CH(CH₂)ᵣO—, —O(CH₂)_{q}CH≡CH(CH₂)ᵣO—, —S(O)ᵥ(CH₂)_{q}CH═CH(CH₂)ᵣ—, S(O)ᵥ(CH₂)_{q}CH≡CH(CH₂)ᵣ—, —(CH₂)_{q}CH═CH(CH₂)ᵣS(O)ᵥ—, (CH₂)_{q}CH≡CH(CH₂)ᵣS(O)ᵥ—, —O(CH₂)_{q}CH═CH(CH₂)ᵣS(O)ᵥ—, —O(CH₂)_{q}CH≡CH(CH₂)ᵣS(O)ᵥ—, —S(O)ᵥ(CH₂)_{q}CH═CH(CH₂)ᵣO—, S(O)ᵥ(CH₂)_{q}CH≡CH(CH₂)ᵣO—, —C(CH₂)_{q}S(CH₂)ᵣ —, —C(CH₂)_{q}O(CH₂)ᵣ—, —C(O)NR¹⁹S(O)₂(CH₂)_{q}—, and —(CH₂)_{q}S(O)₂NR¹⁹C(O)—; or L₃ is L₄-L₅-L₆;
L₄ and L₆ are each independently selected from the group consisting of —(CH₂)_{q}—, —O(CH₂)_{q}—, —S(CH₂)_{q}—, —NR¹⁹(CH₂)_{q}—, —(CH₂)_{q}NR²⁰—, —(CH₂)_{q}O—, —(CH₂)_{q}S—, —(CH₂)_{q}C(O)—, —C(O)(CH₂)_{q}—, —(CH₂)_{q}C(O)NR¹⁹—, —NR¹⁹C(O)(CH₂)_{q}—, —(CH₂)_{q}CH═CH(CH₂)ᵣ—, —O(CH₂)_{q}CH═CH(CH₂)ᵣ—, —(CH₂)_{q}CH═CH(CH₂)ᵣO—, —S(CH₂)_{q}CH═CH(CH₂)ᵣ—, —(CH₂)_{q}CH═CH(CH₂)ᵣS—, —O(CH₂)_{q}CH═CH(CH₂)ᵣS—, and —S(CH₂)_{q}CH═CH(CH₂)ᵣO—;
L⁵ is a C₂₋₆ heterocyclyl, an C₆₋₁₀ aryl or a C₁₋₉ heteroaryl;
each of the oxo group in L₃, L₄, L₅ and L₆ can be independently optionally replaced with a thiocarbonyl group (—C(S)—), an oxetane group, or an imine group (—C(═NR¹⁹)—)
q and r are each independently an integer selected from 0 to 10;
v is 0, 1 or 2;
R¹⁹ and R²⁰ are each independently selected from the group consisting of hydrogen, C₁₋₆ alkyl, C₃₋₁₀ heteroalkyl, C₃₋₆ cycloalkyl, C₆₋₁₀ aryl or a C₁₋₅ heteroaryl, and C₂₋₆ heterocyclyl; or
R¹⁹ and R²⁰ can be connected to form a ring;
Q is a moiety capable of forming a covalent bond with a nucleophile

2. A compound of Formula VIIA in which Q is any of the following moieties:

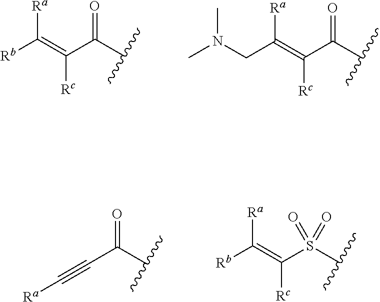

3. The compound or a pharmaceutically acceptable salt thereof of claim 1 wherein the compound is selected from the group consisting of

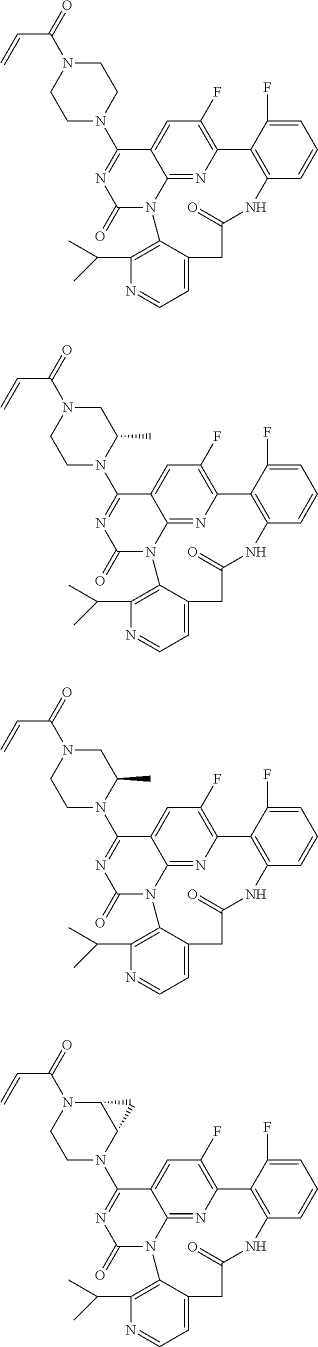 [SMILES: C=CC(=O)N1CCN(c2nc(=O)n3c4nc(c(F)cc24)-c2c(F)cccc2NC(=O)Cc2ccnc(C(C)C)c2-3)CC1 ; C=CC(=O)N1CCN(c2nc(=O)n3c4nc(c(F)cc24)-c2c(F)cccc2NC(=O)Cc2ccnc(C(C)C)c2-3)[C@@H](C)C1 ; C=CC(=O)N1CCN(c2nc(=O)n3c4nc(c(F)cc24)-c2c(F)cccc2NC(=O)Cc2ccnc(C(C)C)c2-3)[C@H](C)C1 ; C=CC(=O)N1CCN(c2nc(=O)n3c4nc(c(F)cc24)-c2c(F)cccc2NC(=O)Cc2ccnc(C(C)C)c2-3)[C@H]2C[C@H]21]

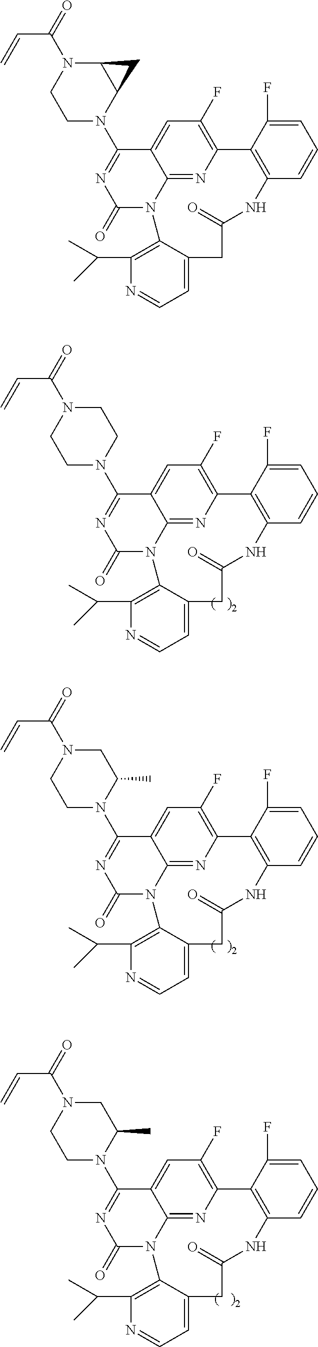

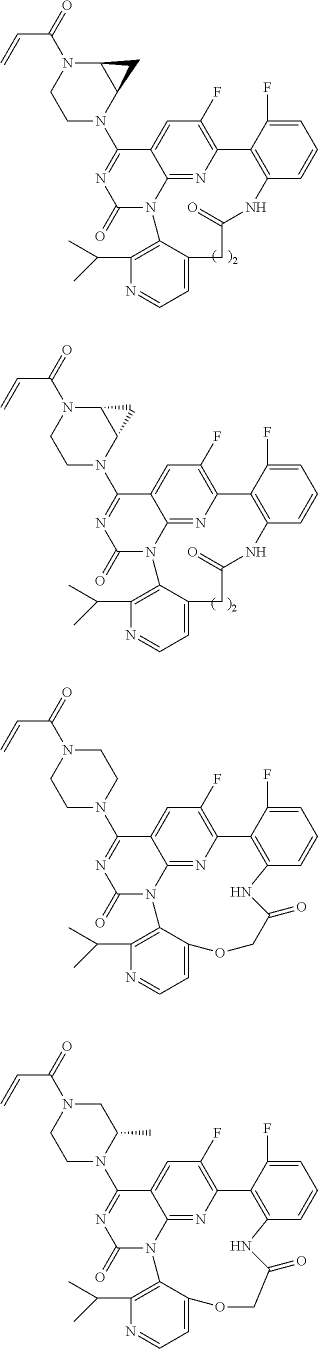

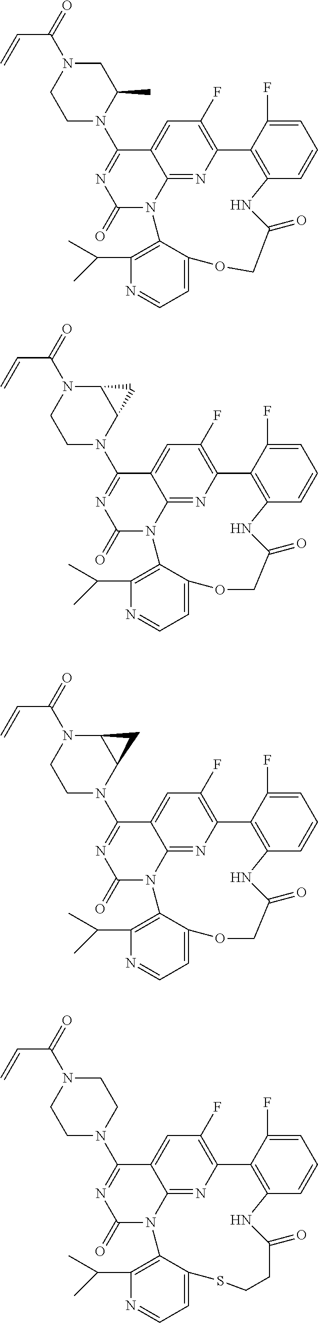 [SMILES: C=CC(=O)N1CCN(c2nc(=O)n3c4nc(c(F)cc24)-c2c(F)cccc2NC(=O)COc2ccnc(C(C)C)c2-3)[C@H](C)C1 ; C=CC(=O)N1CCN(c2nc(=O)n3c4nc(c(F)cc24)-c2c(F)cccc2NC(=O)COc2ccnc(C(C)C)c2-3)[C@H]2C[C@H]21 ; C=CC(=O)N1CCN(c2nc(=O)n3c4nc(c(F)cc24)-c2c(F)cccc2NC(=O)COc2ccnc(C(C)C)c2-3)[C@@H]2C[C@@H]21 ; C=CC(=O)N1CCN(c2nc(=O)n3c4nc(c(F)cc24)-c2c(F)cccc2NC(=O)CCSc2ccnc(C(C)C)c2-3)CC1]

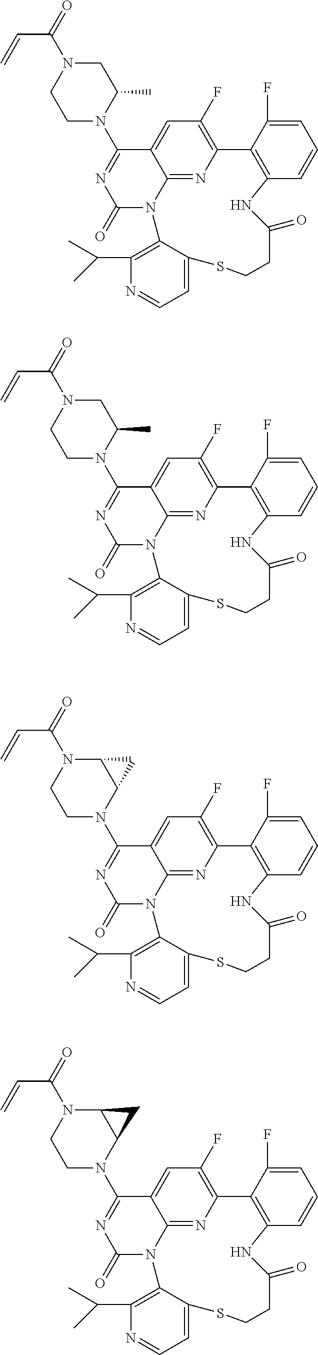 [SMILES: C=CC(=O)N1CCN(c2nc(=O)n3c4nc(c(F)cc24)-c2c(F)cccc2NC(=O)CCSc2ccnc(C(C)C)c2-3)[C@@H](C)C1 ; C=CC(=O)N1CCN(c2nc(=O)n3c4nc(c(F)cc24)-c2c(F)cccc2NC(=O)CCSc2ccnc(C(C)C)c2-3)[C@H](C)C1 ; C=CC(=O)N1CCN(c2nc(=O)n3c4nc(c(F)cc24)-c2c(F)cccc2NC(=O)CCSc2ccnc(C(C)C)c2-3)[C@H]2C[C@H]21 ; C=CC(=O)N1CCN(c2nc(=O)n3c4nc(c(F)cc24)-c2c(F)cccc2NC(=O)CCSc2ccnc(C(C)C)c2-3)[C@@H]2C[C@@H]21]

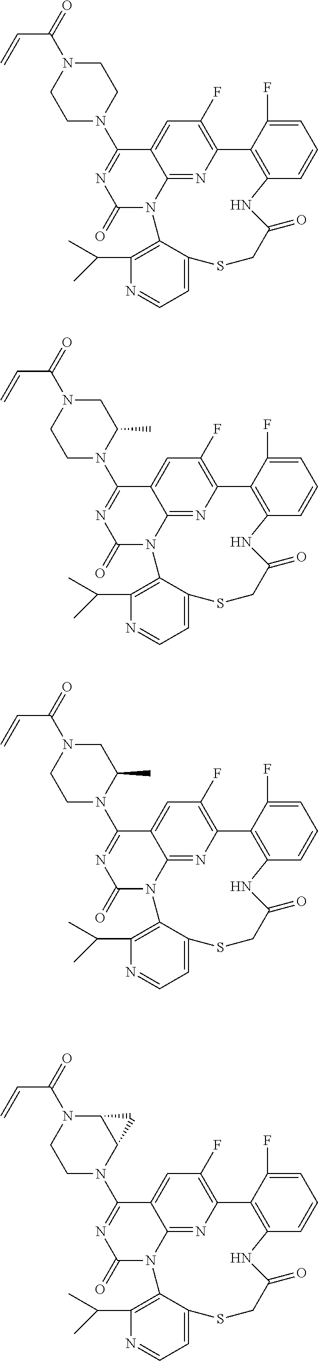 [SMILES: C=CC(=O)N1CCN(c2nc(=O)n3c4nc(c(F)cc24)-c2c(F)cccc2NC(=O)CSc2ccnc(C(C)C)c2-3)CC1 ; C=CC(=O)N1CCN(c2nc(=O)n3c4nc(c(F)cc24)-c2c(F)cccc2NC(=O)CSc2ccnc(C(C)C)c2-3)[C@@H](C)C1 ; C=CC(=O)N1CCN(c2nc(=O)n3c4nc(c(F)cc24)-c2c(F)cccc2NC(=O)CSc2ccnc(C(C)C)c2-3)[C@H](C)C1 ; C=CC(=O)N1CCN(c2nc(=O)n3c4nc(c(F)cc24)-c2c(F)cccc2NC(=O)CSc2ccnc(C(C)C)c2-3)[C@H]2C[C@H]21]

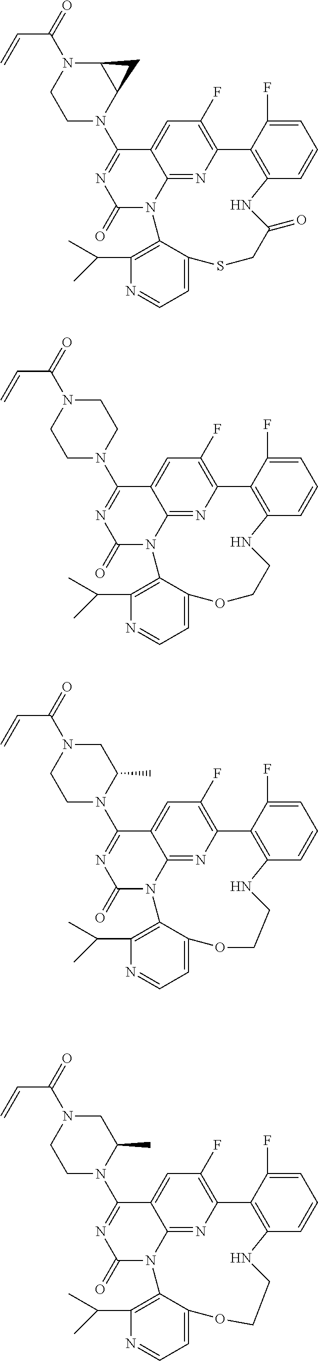 [SMILES: C=CC(=O)N1CCN(c2nc(=O)n3c4nc(c(F)cc24)-c2c(F)cccc2NC(=O)CSc2ccnc(C(C)C)c2-3)[C@@H]2C[C@@H]21 ; C=CC(=O)N1CCN(c2nc(=O)n3c4nc(c(F)cc24)-c2c(F)cccc2NCCOc2ccnc(C(C)C)c2-3)CC1 ; C=CC(=O)N1CCN(c2nc(=O)n3c4nc(c(F)cc24)-c2c(F)cccc2NCCOc2ccnc(C(C)C)c2-3)[C@@H](C)C1 ; C=CC(=O)N1CCN(c2nc(=O)n3c4nc(c(F)cc24)-c2c(F)cccc2NCCOc2ccnc(C(C)C)c2-3)[C@H](C)C1]

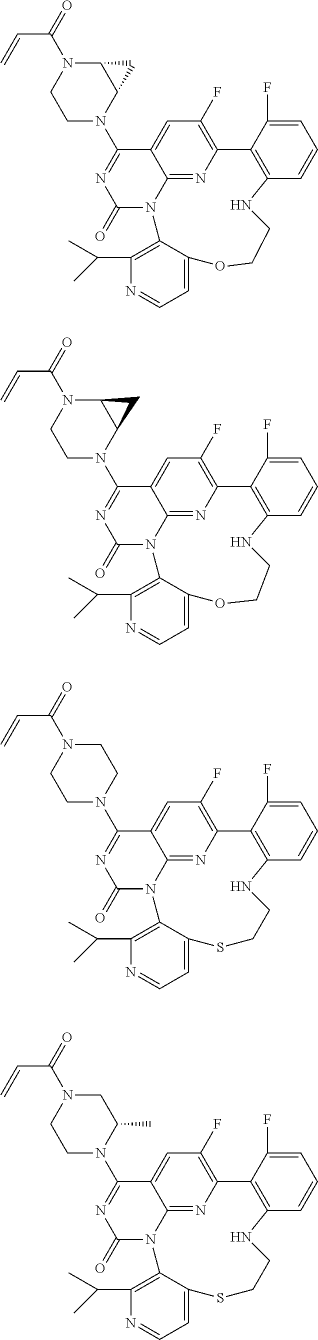 [SMILES: C=CC(=O)N1CCN(c2nc(=O)n3c4nc(c(F)cc24)-c2c(F)cccc2NCCOc2ccnc(C(C)C)c2-3)[C@H]2C[C@H]21 ; C=CC(=O)N1CCN(c2nc(=O)n3c4nc(c(F)cc24)-c2c(F)cccc2NCCOc2ccnc(C(C)C)c2-3)[C@@H]2C[C@@H]21 ; C=CC(=O)N1CCN(c2nc(=O)n3c4nc(c(F)cc24)-c2c(F)cccc2NCCSc2ccnc(C(C)C)c2-3)CC1 ; C=CC(=O)N1CCN(c2nc(=O)n3c4nc(c(F)cc24)-c2c(F)cccc2NCCSc2ccnc(C(C)C)c2-3)[C@@H](C)C1]

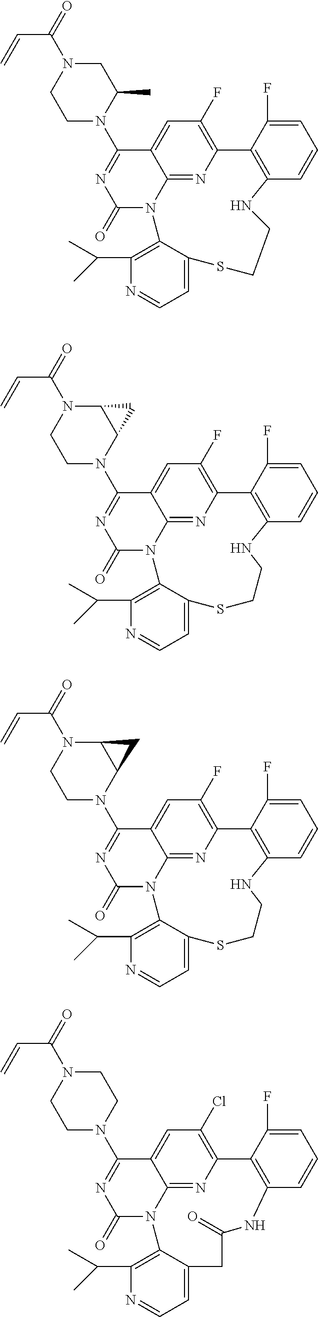 [SMILES: C=CC(=O)N1CCN(c2nc(=O)n3c4nc(c(F)cc24)-c2c(F)cccc2NCCSc2ccnc(C(C)C)c2-3)[C@H](C)C1 ; C=CC(=O)N1CCN(c2nc(=O)n3c4nc(c(F)cc24)-c2c(F)cccc2NCCSc2ccnc(C(C)C)c2-3)[C@H]2C[C@H]21 ; C=CC(=O)N1CCN(c2nc(=O)n3c4nc(c(F)cc24)-c2c(F)cccc2NCCSc2ccnc(C(C)C)c2-3)[C@@H]2C[C@@H]21 ; C=CC(=O)N1CCN(c2nc(=O)n3c4nc(c(Cl)cc24)-c2c(F)cccc2NC(=O)Cc2ccnc(C(C)C)c2-3)CC1]

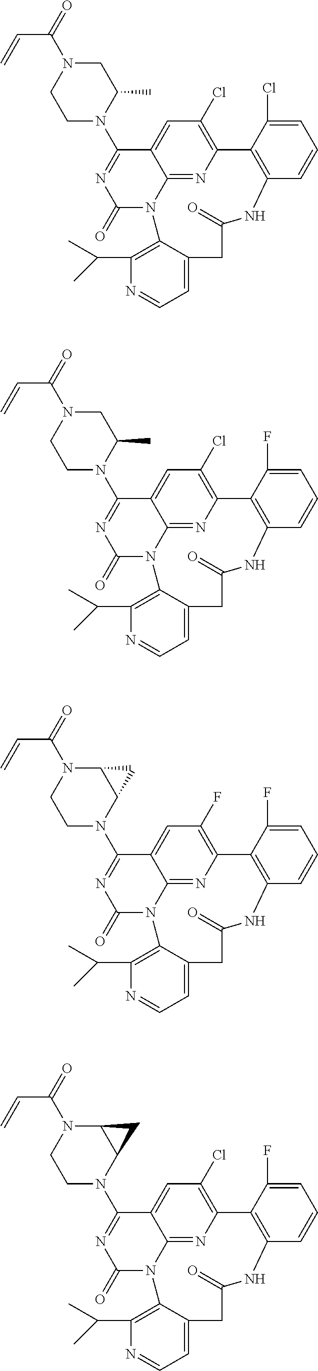 [SMILES: C=CC(=O)N1CCN(c2nc(=O)n3c4nc(c(Cl)cc24)-c2c(Cl)cccc2NC(=O)Cc2ccnc(C(C)C)c2-3)[C@@H](C)C1 ; C=CC(=O)N1CCN(c2nc(=O)n3c4nc(c(Cl)cc24)-c2c(F)cccc2NC(=O)Cc2ccnc(C(C)C)c2-3)[C@H](C)C1 ; C=CC(=O)N1CCN(c2nc(=O)n3c4nc(c(F)cc24)-c2c(F)cccc2NC(=O)Cc2ccnc(C(C)C)c2-3)[C@H]2C[C@H]21 ; C=CC(=O)N1CCN(c2nc(=O)n3c4nc(c(Cl)cc24)-c2c(F)cccc2NC(=O)Cc2ccnc(C(C)C)c2-3)[C@@H]2C[C@@H]21]

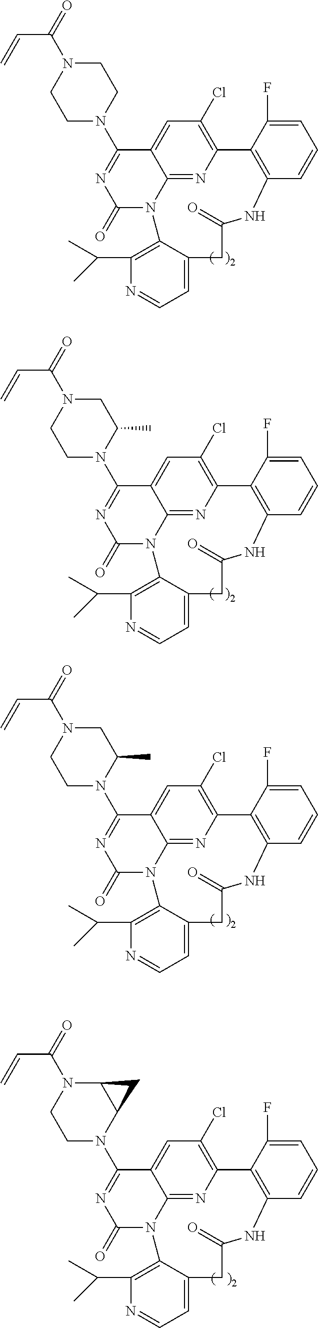

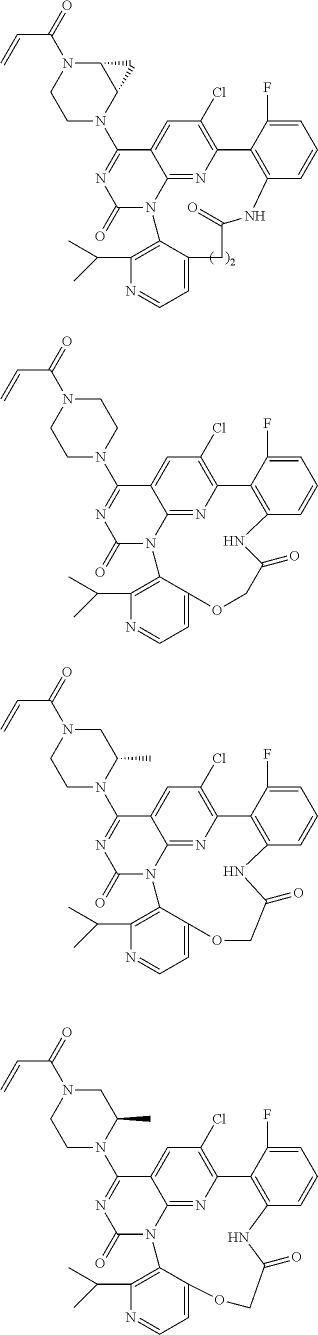

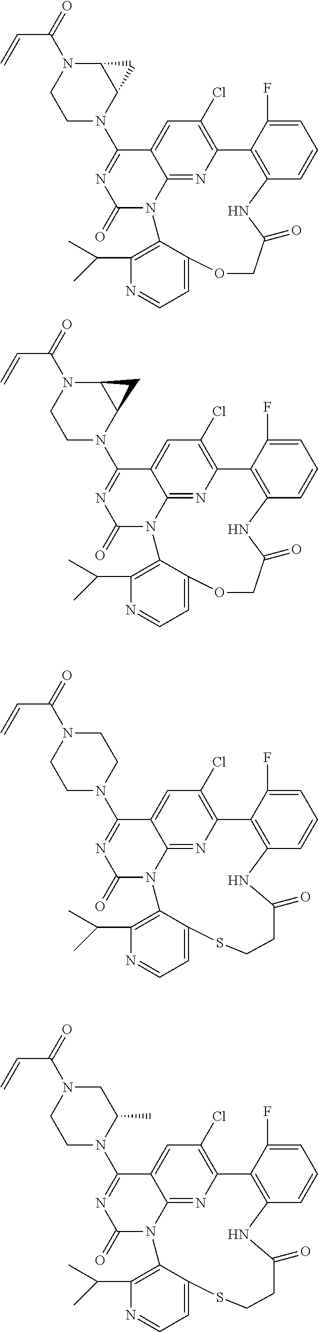

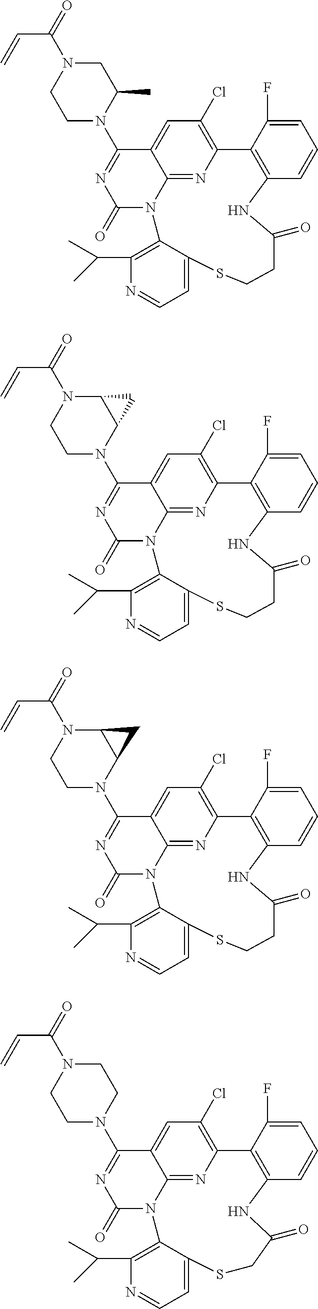 [SMILES: C=CC(=O)N1CCN(c2nc(=O)n3c4nc(c(Cl)cc24)-c2c(F)cccc2NC(=O)CCSc2ccnc(C(C)C)c2-3)[C@H](C)C1 ; C=CC(=O)N1CCN(c2nc(=O)n3c4nc(c(Cl)cc24)-c2c(F)cccc2NC(=O)CCSc2ccnc(C(C)C)c2-3)[C@H]2C[C@H]21 ; C=CC(=O)N1CCN(c2nc(=O)n3c4nc(c(Cl)cc24)-c2c(F)cccc2NC(=O)CCSc2ccnc(C(C)C)c2-3)[C@@H]2C[C@@H]21 ; C=CC(=O)N1CCN(c2nc(=O)n3c4nc(c(Cl)cc24)-c2c(F)cccc2NC(=O)CSc2ccnc(C(C)C)c2-3)CC1]

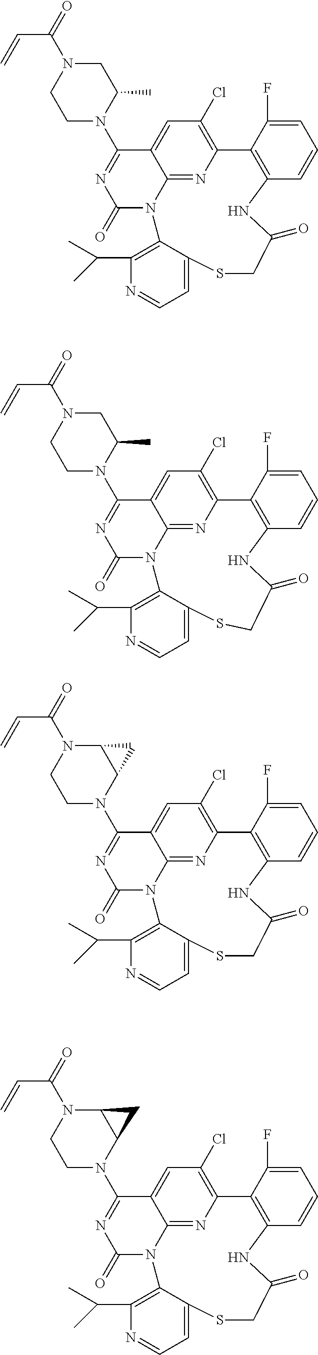 [SMILES: C=CC(=O)N1CCN(c2nc(=O)n3c4nc(c(Cl)cc24)-c2c(F)cccc2NC(=O)CSc2ccnc(C(C)C)c2-3)[C@@H](C)C1 ; C=CC(=O)N1CCN(c2nc(=O)n3c4nc(c(Cl)cc24)-c2c(F)cccc2NC(=O)CSc2ccnc(C(C)C)c2-3)[C@H](C)C1 ; C=CC(=O)N1CCN(c2nc(=O)n3c4nc(c(Cl)cc24)-c2c(F)cccc2NC(=O)CSc2ccnc(C(C)C)c2-3)[C@H]2C[C@H]21 ; C=CC(=O)N1CCN(c2nc(=O)n3c4nc(c(Cl)cc24)-c2c(F)cccc2NC(=O)CSc2ccnc(C(C)C)c2-3)[C@@H]2C[C@@H]21]

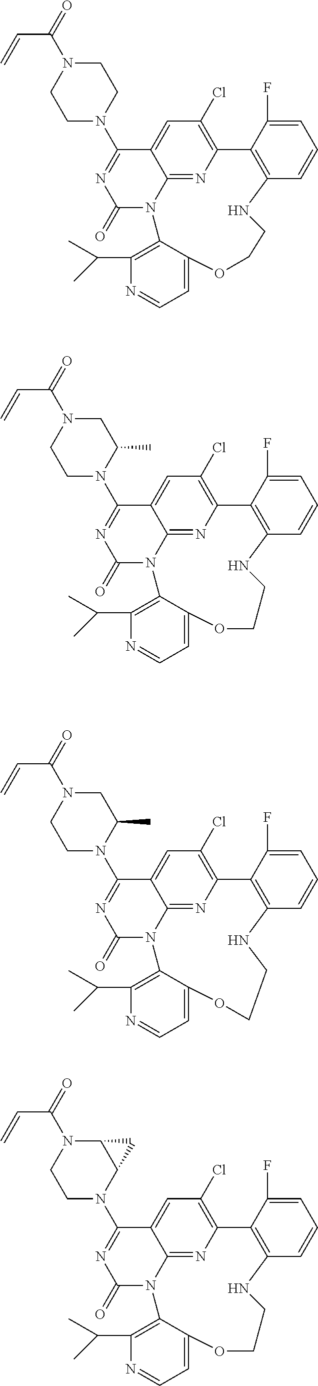 [SMILES: C=CC(=O)N1CCN(c2nc(=O)n3c4nc(c(Cl)cc24)-c2c(F)cccc2NCCOc2ccnc(C(C)C)c2-3)CC1 ; C=CC(=O)N1CCN(c2nc(=O)n3c4nc(c(Cl)cc24)-c2c(F)cccc2NCCOc2ccnc(C(C)C)c2-3)[C@@H](C)C1 ; C=CC(=O)N1CCN(c2nc(=O)n3c4nc(c(Cl)cc24)-c2c(F)cccc2NCCOc2ccnc(C(C)C)c2-3)[C@H](C)C1 ; C=CC(=O)N1CCN(c2nc(=O)n3c4nc(c(Cl)cc24)-c2c(F)cccc2NCCOc2ccnc(C(C)C)c2-3)[C@H]2C[C@H]21]

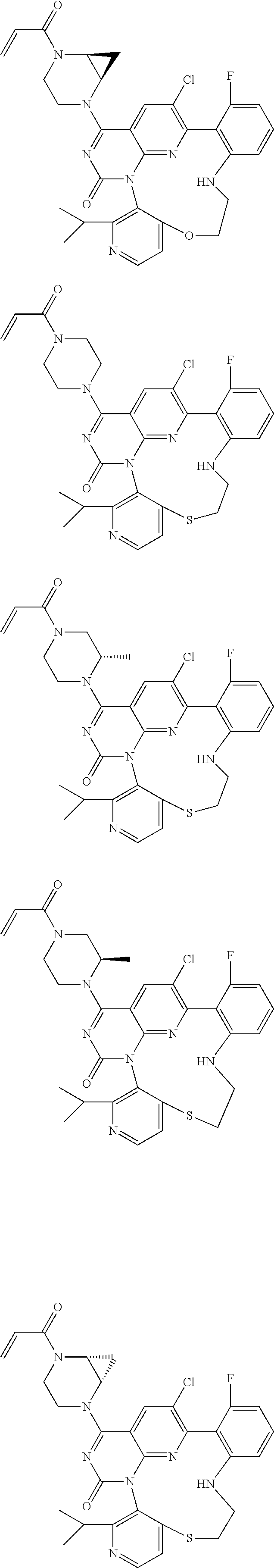 [SMILES: C=CC(=O)N1CCN(c2nc(=O)n3c4nc(c(Cl)cc24)-c2c(F)cccc2NCCOc2ccnc(C(C)C)c2-3)[C@@H]2C[C@@H]21 ; C=CC(=O)N1CCN(c2nc(=O)n3c4nc(c(Cl)cc24)-c2c(F)cccc2NCCSc2ccnc(C(C)C)c2-3)CC1 ; C=CC(=O)N1CCN(c2nc(=O)n3c4nc(c(Cl)cc24)-c2c(F)cccc2NCCSc2ccnc(C(C)C)c2-3)[C@@H](C)C1 ; C=CC(=O)N1CCN(c2nc(=O)n3c4nc(c(Cl)cc24)-c2c(F)cccc2NCCSc2ccnc(C(C)C)c2-3)[C@H](C)C1 ; C=CC(=O)N1CCN(c2nc(=O)n3c4nc(c(Cl)cc24)-c2c(F)cccc2NCCSc2ccnc(C(C)C)c2-3)[C@H]2C[C@H]21]

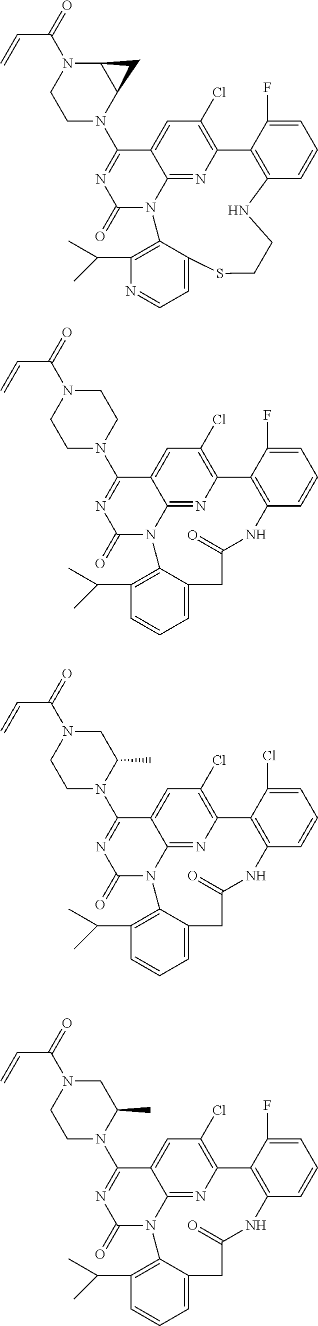 [SMILES: C=CC(=O)N1CCN(c2nc(=O)n3c4nc(c(Cl)cc24)-c2c(F)cccc2NCCSc2ccnc(C(C)C)c2-3)[C@@H]2C[C@@H]21 ; C=CC(=O)N1CCN(c2nc(=O)n3c4nc(c(Cl)cc24)-c2c(F)cccc2NC(=O)Cc2cccc(C(C)C)c2-3)CC1 ; C=CC(=O)N1CCN(c2nc(=O)n3c4nc(c(Cl)cc24)-c2c(Cl)cccc2NC(=O)Cc2cccc(C(C)C)c2-3)[C@@H](C)C1 ; C=CC(=O)N1CCN(c2nc(=O)n3c4nc(c(Cl)cc24)-c2c(F)cccc2NC(=O)Cc2cccc(C(C)C)c2-3)[C@H](C)C1]

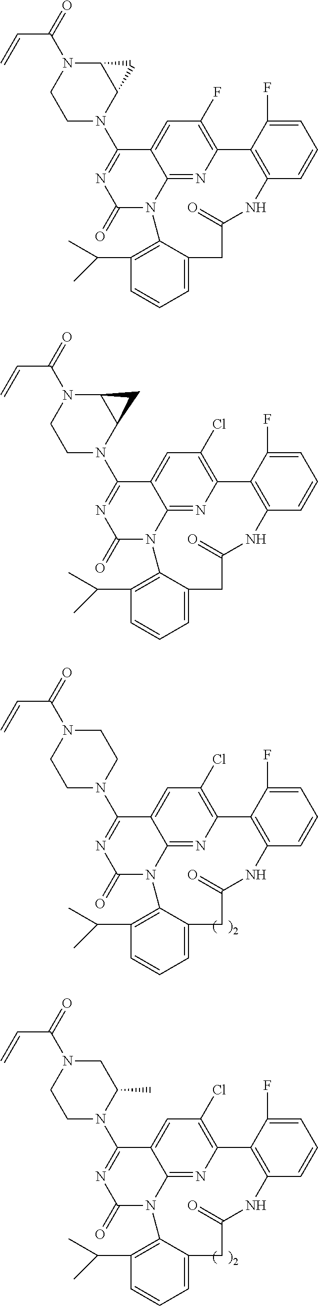

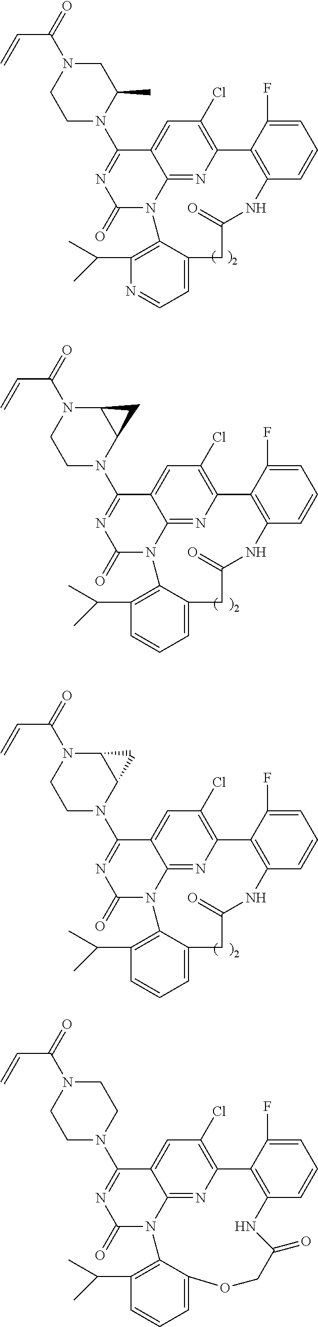

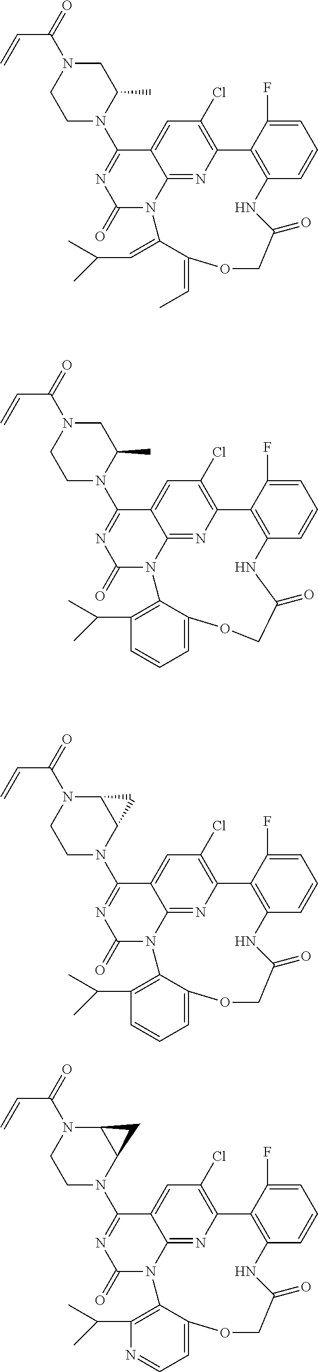 [SMILES: C=CC(=O)N1CCN(c2nc(=O)n3c4nc(c(Cl)cc24)-c2c(F)cccc2NC(=O)COC(=C/C)/C3=C/C(C)C)[C@@H](C)C1 ; C=CC(=O)N1CCN(c2nc(=O)n3c4nc(c(Cl)cc24)-c2c(F)cccc2NC(=O)COc2cccc(C(C)C)c2-3)[C@H](C)C1 ; C=CC(=O)N1CCN(c2nc(=O)n3c4nc(c(Cl)cc24)-c2c(F)cccc2NC(=O)COc2cccc(C(C)C)c2-3)[C@H]2C[C@H]21 ; C=CC(=O)N1CCN(c2nc(=O)n3c4nc(c(Cl)cc24)-c2c(F)cccc2NC(=O)COc2ccnc(C(C)C)c2-3)[C@@H]2C[C@@H]21]

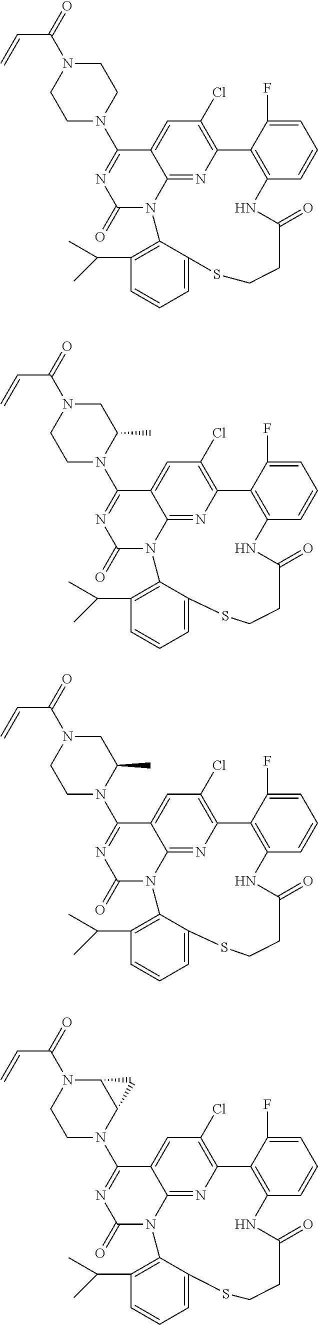 [SMILES: C=CC(=O)N1CCN(c2nc(=O)n3c4nc(c(Cl)cc24)-c2c(F)cccc2NC(=O)CCSc2cccc(C(C)C)c2-3)CC1 ; C=CC(=O)N1CCN(c2nc(=O)n3c4nc(c(Cl)cc24)-c2c(F)cccc2NC(=O)CCSc2cccc(C(C)C)c2-3)[C@@H](C)C1 ; C=CC(=O)N1CCN(c2nc(=O)n3c4nc(c(Cl)cc24)-c2c(F)cccc2NC(=O)CCSc2cccc(C(C)C)c2-3)[C@H](C)C1 ; C=CC(=O)N1CCN(c2nc(=O)n3c4nc(c(Cl)cc24)-c2c(F)cccc2NC(=O)CCSc2cccc(C(C)C)c2-3)[C@H]2C[C@H]21]

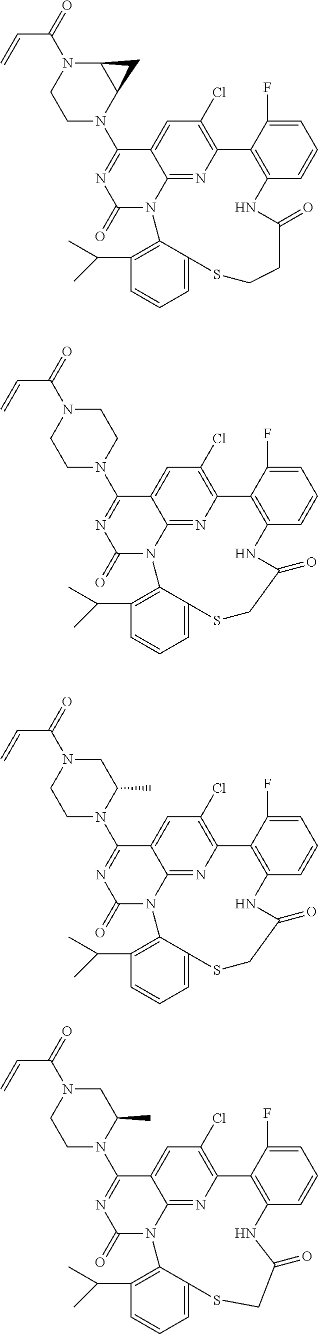 [SMILES: C=CC(=O)N1CCN(c2nc(=O)n3c4nc(c(Cl)cc24)-c2c(F)cccc2NC(=O)CCSc2cccc(C(C)C)c2-3)[C@@H]2C[C@@H]21 ; C=CC(=O)N1CCN(c2nc(=O)n3c4nc(c(Cl)cc24)-c2c(F)cccc2NC(=O)CSc2cccc(C(C)C)c2-3)CC1 ; C=CC(=O)N1CCN(c2nc(=O)n3c4nc(c(Cl)cc24)-c2c(F)cccc2NC(=O)CSc2cccc(C(C)C)c2-3)[C@@H](C)C1 ; C=CC(=O)N1CCN(c2nc(=O)n3c4nc(c(Cl)cc24)-c2c(F)cccc2NC(=O)CSc2cccc(C(C)C)c2-3)[C@H](C)C1]

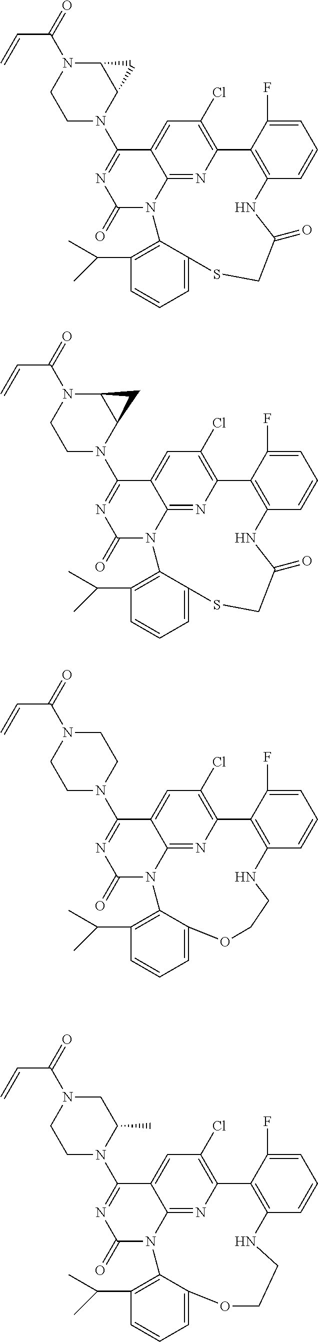

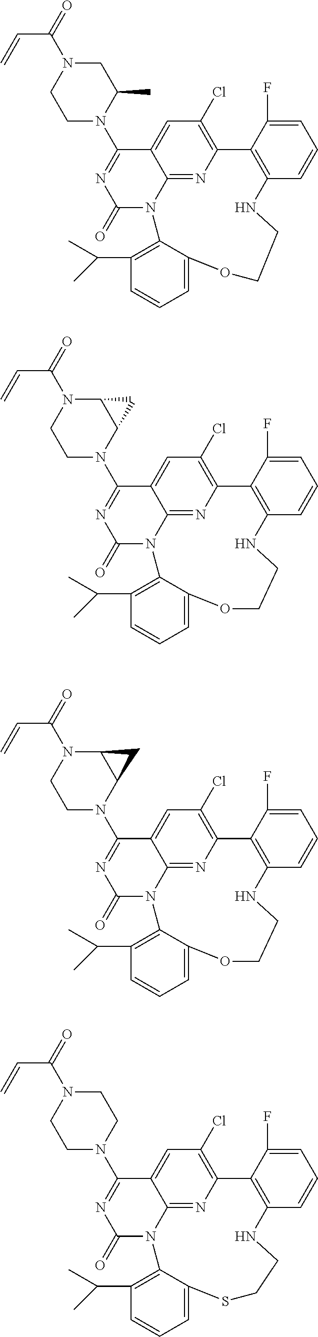 [SMILES: C=CC(=O)N1CCN(c2nc(=O)n3c4nc(c(Cl)cc24)-c2c(F)cccc2NCCOc2cccc(C(C)C)c2-3)[C@H](C)C1 ; C=CC(=O)N1CCN(c2nc(=O)n3c4nc(c(Cl)cc24)-c2c(F)cccc2NCCOc2cccc(C(C)C)c2-3)[C@H]2C[C@H]21 ; C=CC(=O)N1CCN(c2nc(=O)n3c4nc(c(Cl)cc24)-c2c(F)cccc2NCCOc2cccc(C(C)C)c2-3)[C@@H]2C[C@@H]21 ; C=CC(=O)N1CCN(c2nc(=O)n3c4nc(c(Cl)cc24)-c2c(F)cccc2NCCSc2cccc(C(C)C)c2-3)CC1]

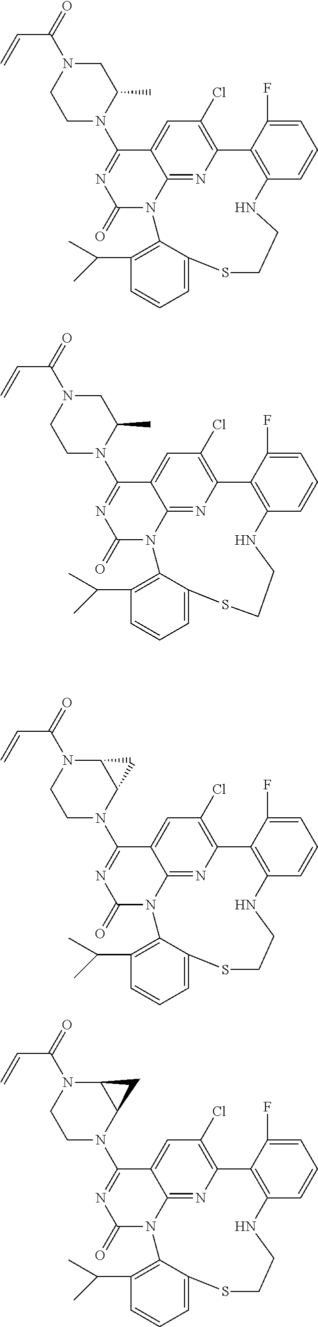 [SMILES: C=CC(=O)N1CCN(c2nc(=O)n3c4nc(c(Cl)cc24)-c2c(F)cccc2NCCSc2cccc(C(C)C)c2-3)[C@@H](C)C1 ; C=CC(=O)N1CCN(c2nc(=O)n3c4nc(c(Cl)cc24)-c2c(F)cccc2NCCSc2cccc(C(C)C)c2-3)[C@H](C)C1 ; C=CC(=O)N1CCN(c2nc(=O)n3c4nc(c(Cl)cc24)-c2c(F)cccc2NCCSc2cccc(C(C)C)c2-3)[C@H]2C[C@H]21 ; C=CC(=O)N1CCN(c2nc(=O)n3c4nc(c(Cl)cc24)-c2c(F)cccc2NCCSc2cccc(C(C)C)c2-3)[C@@H]2C[C@@H]21]

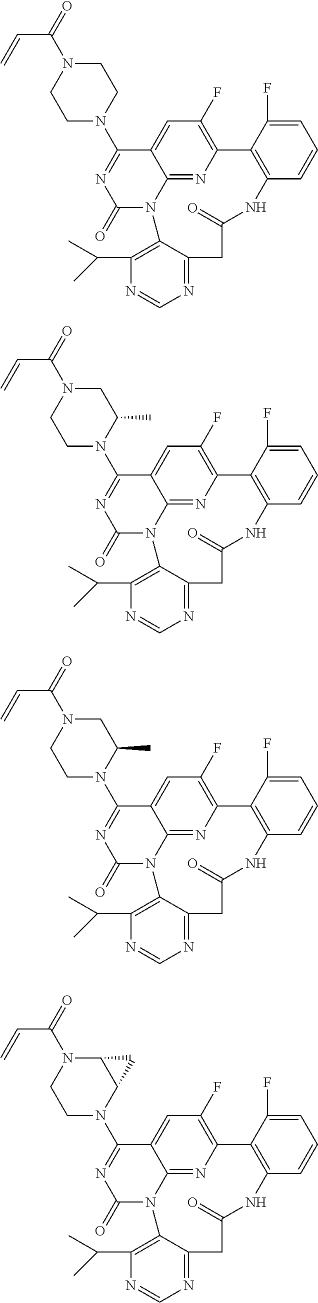 [SMILES: C=CC(=O)N1CCN(c2nc(=O)n3c4nc(c(F)cc24)-c2c(F)cccc2NC(=O)Cc2ncnc(C(C)C)c2-3)CC1 ; C=CC(=O)N1CCN(c2nc(=O)n3c4nc(c(F)cc24)-c2c(F)cccc2NC(=O)Cc2ncnc(C(C)C)c2-3)[C@@H](C)C1 ; C=CC(=O)N1CCN(c2nc(=O)n3c4nc(c(F)cc24)-c2c(F)cccc2NC(=O)Cc2ncnc(C(C)C)c2-3)[C@H](C)C1 ; C=CC(=O)N1CCN(c2nc(=O)n3c4nc(c(F)cc24)-c2c(F)cccc2NC(=O)Cc2ncnc(C(C)C)c2-3)[C@H]2C[C@H]21]

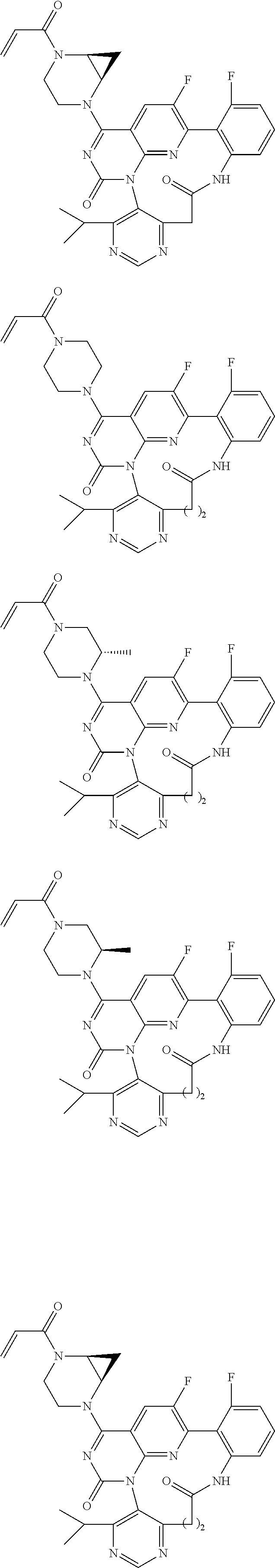

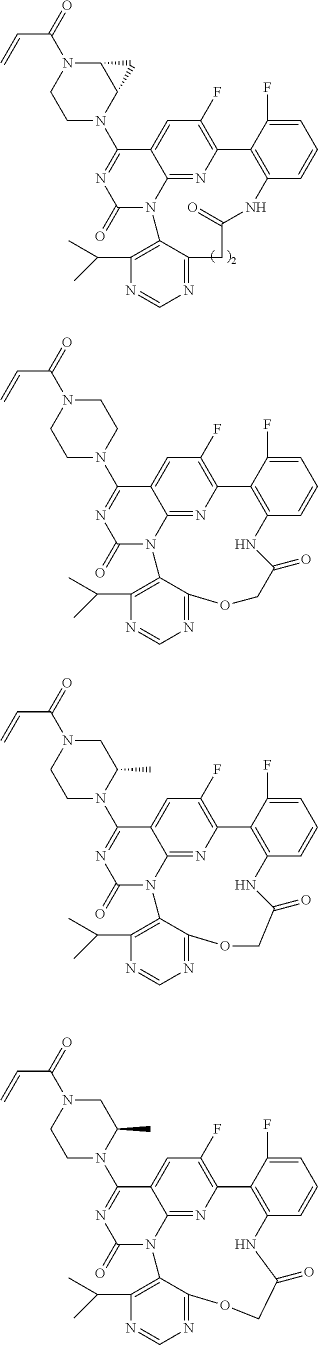

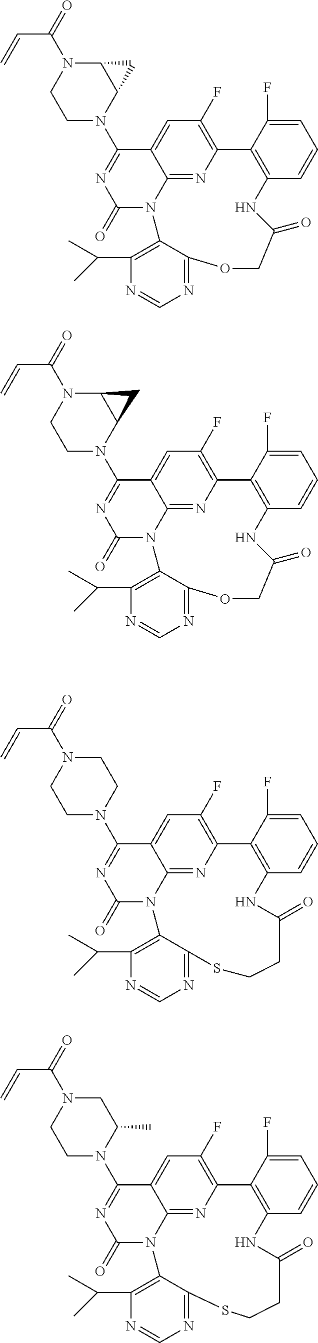 [SMILES: C=CC(=O)N1CCN(c2nc(=O)n3c4nc(c(F)cc24)-c2c(F)cccc2NC(=O)COc2ncnc(C(C)C)c2-3)[C@H]2C[C@H]21 ; C=CC(=O)N1CCN(c2nc(=O)n3c4nc(c(F)cc24)-c2c(F)cccc2NC(=O)COc2ncnc(C(C)C)c2-3)[C@@H]2C[C@@H]21 ; C=CC(=O)N1CCN(c2nc(=O)n3c4nc(c(F)cc24)-c2c(F)cccc2NC(=O)CCSc2ncnc(C(C)C)c2-3)CC1 ; C=CC(=O)N1CCN(c2nc(=O)n3c4nc(c(F)cc24)-c2c(F)cccc2NC(=O)CCSc2ncnc(C(C)C)c2-3)[C@@H](C)C1]

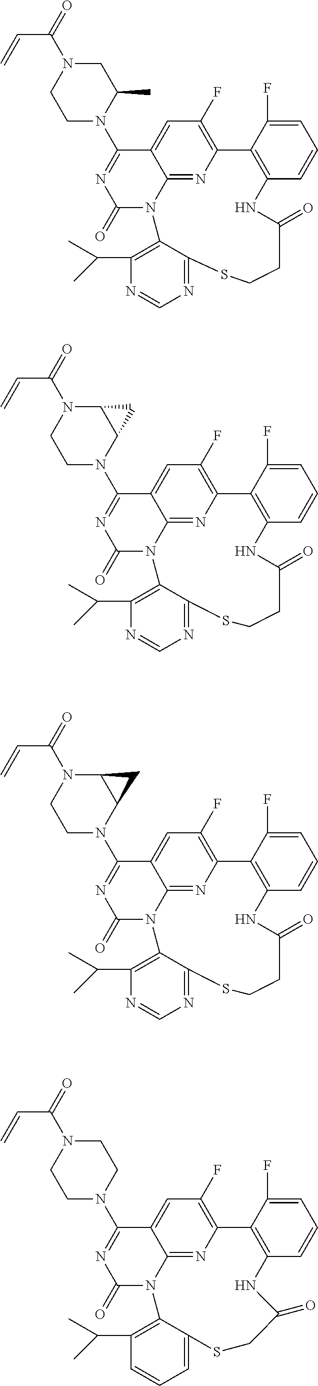 [SMILES: C=CC(=O)N1CCN(c2nc(=O)n3c4nc(c(F)cc24)-c2c(F)cccc2NC(=O)CCSc2ncnc(C(C)C)c2-3)[C@H](C)C1 ; C=CC(=O)N1CCN(c2nc(=O)n3c4nc(c(F)cc24)-c2c(F)cccc2NC(=O)CCSc2ncnc(C(C)C)c2-3)[C@H]2C[C@H]21 ; C=CC(=O)N1CCN(c2nc(=O)n3c4nc(c(F)cc24)-c2c(F)cccc2NC(=O)CCSc2ncnc(C(C)C)c2-3)[C@@H]2C[C@@H]21 ; C=CC(=O)N1CCN(c2nc(=O)n3c4nc(c(F)cc24)-c2c(F)cccc2NC(=O)CSc2cccc(C(C)C)c2-3)CC1]

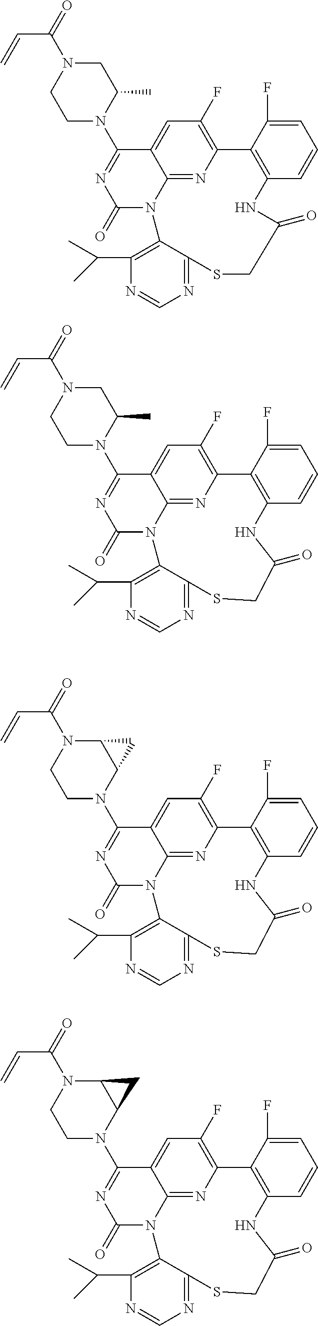 [SMILES: C=CC(=O)N1CCN(c2nc(=O)n3c4nc(c(F)cc24)-c2c(F)cccc2NC(=O)CSc2ncnc(C(C)C)c2-3)[C@@H](C)C1 ; C=CC(=O)N1CCN(c2nc(=O)n3c4nc(c(F)cc24)-c2c(F)cccc2NC(=O)CSc2ncnc(C(C)C)c2-3)[C@H](C)C1 ; C=CC(=O)N1CCN(c2nc(=O)n3c4nc(c(F)cc24)-c2c(F)cccc2NC(=O)CSc2ncnc(C(C)C)c2-3)[C@H]2C[C@H]21 ; C=CC(=O)N1CCN(c2nc(=O)n3c4nc(c(F)cc24)-c2c(F)cccc2NC(=O)CSc2ncnc(C(C)C)c2-3)[C@@H]2C[C@@H]21]

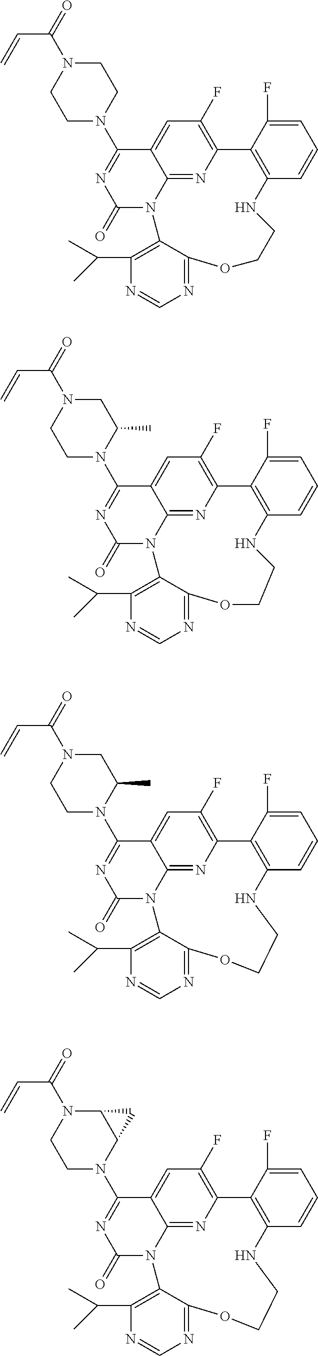 [SMILES: C=CC(=O)N1CCN(c2nc(=O)n3c4nc(c(F)cc24)-c2c(F)cccc2NCCOc2ncnc(C(C)C)c2-3)CC1 ; C=CC(=O)N1CCN(c2nc(=O)n3c4nc(c(F)cc24)-c2c(F)cccc2NCCOc2ncnc(C(C)C)c2-3)[C@@H](C)C1 ; C=CC(=O)N1CCN(c2nc(=O)n3c4nc(c(F)cc24)-c2c(F)cccc2NCCOc2ncnc(C(C)C)c2-3)[C@H](C)C1 ; C=CC(=O)N1CCN(c2nc(=O)n3c4nc(c(F)cc24)-c2c(F)cccc2NCCOc2ncnc(C(C)C)c2-3)[C@H]2C[C@H]21]

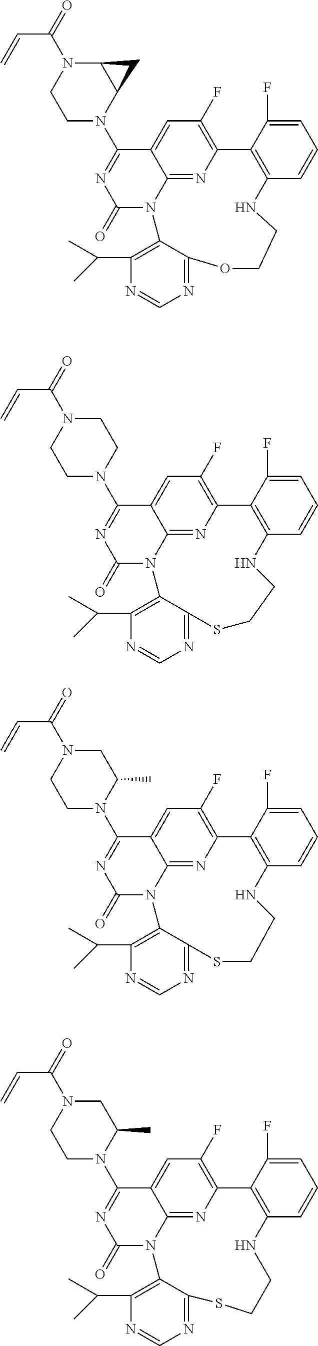 [SMILES: C=CC(=O)N1CCN(c2nc(=O)n3c4nc(c(F)cc24)-c2c(F)cccc2NCCOc2ncnc(C(C)C)c2-3)[C@@H]2C[C@@H]21 ; C=CC(=O)N1CCN(c2nc(=O)n3c4nc(c(F)cc24)-c2c(F)cccc2NCCSc2ncnc(C(C)C)c2-3)CC1 ; C=CC(=O)N1CCN(c2nc(=O)n3c4nc(c(F)cc24)-c2c(F)cccc2NCCSc2ncnc(C(C)C)c2-3)[C@@H](C)C1 ; C=CC(=O)N1CCN(c2nc(=O)n3c4nc(c(F)cc24)-c2c(F)cccc2NCCSc2ncnc(C(C)C)c2-3)[C@H](C)C1]

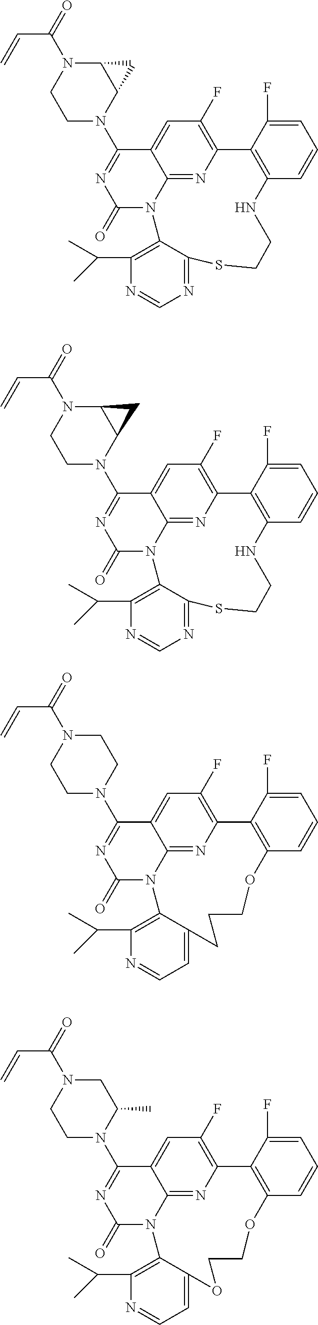 [SMILES: C=CC(=O)N1CCN(c2nc(=O)n3c4nc(c(F)cc24)-c2c(F)cccc2NCCSc2ncnc(C(C)C)c2-3)[C@H]2C[C@H]21 ; C=CC(=O)N1CCN(c2nc(=O)n3c4nc(c(F)cc24)-c2c(F)cccc2NCCSc2ncnc(C(C)C)c2-3)[C@@H]2C[C@@H]21 ; C=CC(=O)N1CCN(c2nc(=O)n3c4nc(c(F)cc24)-c2c(F)cccc2OCCCc2ccnc(C(C)C)c2-3)CC1 ; C=CC(=O)N1CCN(c2nc(=O)n3c4nc(c(F)cc24)-c2c(F)cccc2OCCOc2ccnc(C(C)C)c2-3)[C@@H](C)C1]

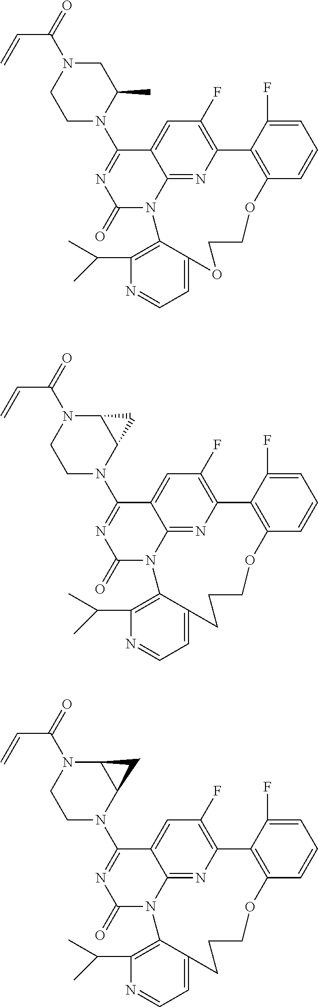 [SMILES: C=CC(=O)N1CCN(c2nc(=O)n3c4nc(c(F)cc24)-c2c(F)cccc2OCCOc2ccnc(C(C)C)c2-3)[C@H](C)C1 ; C=CC(=O)N1CCN(c2nc(=O)n3c4nc(c(F)cc24)-c2c(F)cccc2OCCCc2ccnc(C(C)C)c2-3)[C@H]2C[C@H]21 ; C=CC(=O)N1CCN(c2nc(=O)n3c4nc(c(F)cc24)-c2c(F)cccc2OCCCc2ccnc(C(C)C)c2-3)[C@@H]2C[C@@H]21]

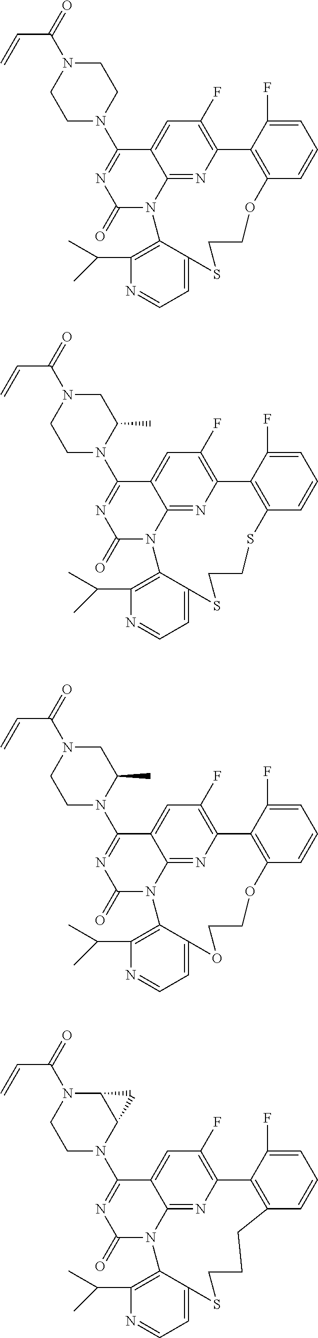 [SMILES: C=CC(=O)N1CCN(c2nc(=O)n3c4nc(c(F)cc24)-c2c(F)cccc2OCCSc2ccnc(C(C)C)c2-3)CC1 ; C=CC(=O)N1CCN(c2nc(=O)n3c4nc(c(F)cc24)-c2c(F)cccc2SCCSc2ccnc(C(C)C)c2-3)[C@@H](C)C1 ; C=CC(=O)N1CCN(c2nc(=O)n3c4nc(c(F)cc24)-c2c(F)cccc2OCCOc2ccnc(C(C)C)c2-3)[C@H](C)C1 ; C=CC(=O)N1CCN(c2nc(=O)n3c4nc(c(F)cc24)-c2c(F)cccc2CCCSc2ccnc(C(C)C)c2-3)[C@H]2C[C@H]21]

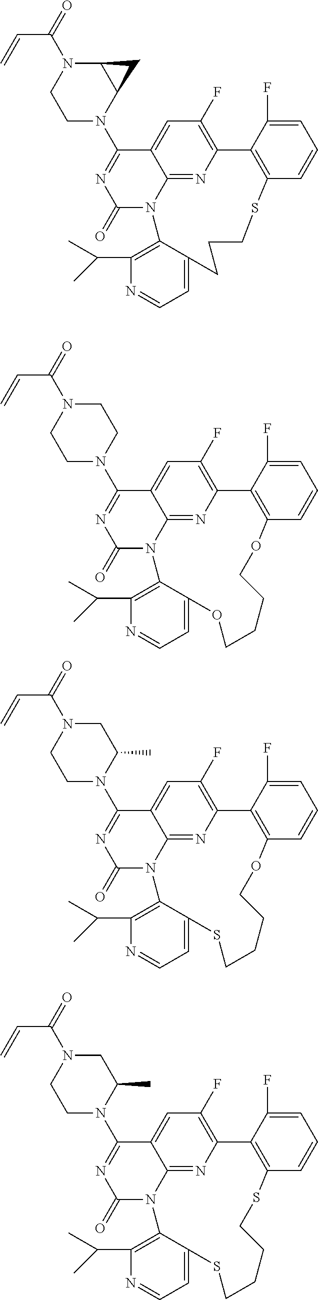

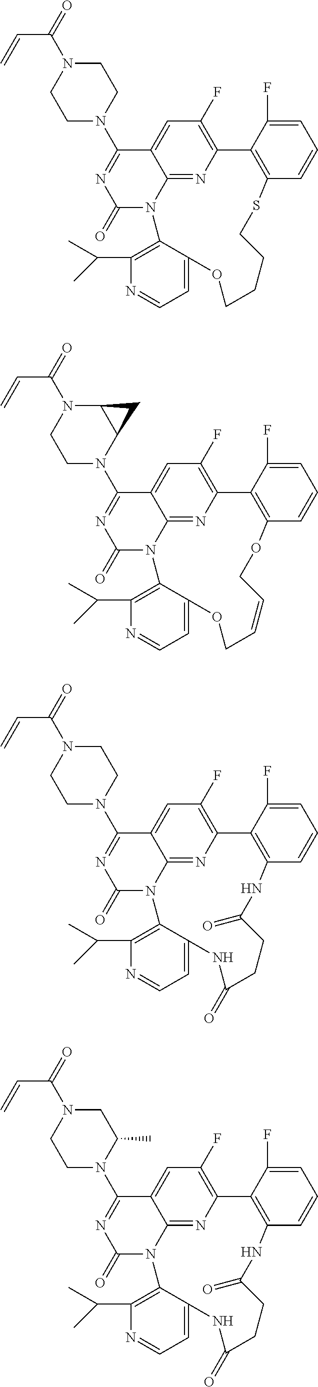 [SMILES: C=CC(=O)N1CCN(c2nc(=O)n3c4nc(c(F)cc24)-c2c(F)cccc2SCCCCOc2ccnc(C(C)C)c2-3)CC1 ; C=CC(=O)N1CCN(c2nc(=O)n3c4nc(c(F)cc24)-c2c(F)cccc2OC/C=C\COc2ccnc(C(C)C)c2-3)[C@@H]2C[C@@H]21 ; C=CC(=O)N1CCN(c2nc(=O)n3c4nc(c(F)cc24)-c2c(F)cccc2NC(=O)CCC(=O)Nc2ccnc(C(C)C)c2-3)CC1 ; C=CC(=O)N1CCN(c2nc(=O)n3c4nc(c(F)cc24)-c2c(F)cccc2NC(=O)CCC(=O)Nc2ccnc(C(C)C)c2-3)[C@@H](C)C1]

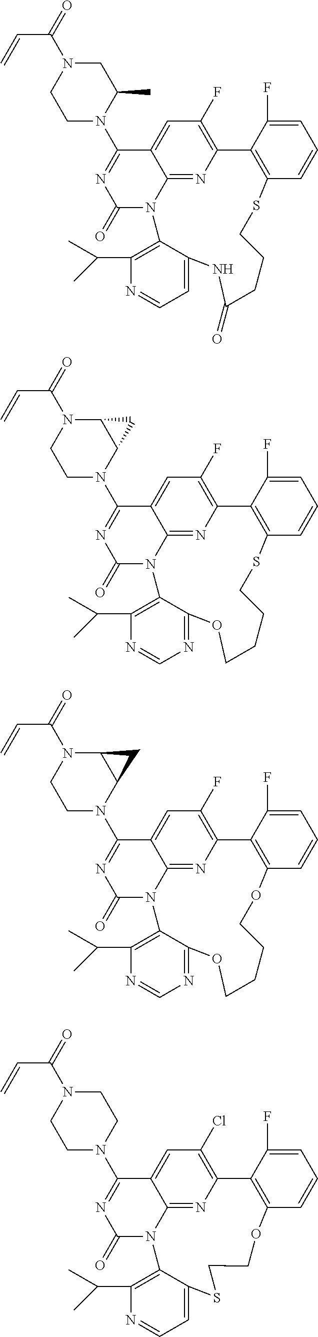 [SMILES: C=CC(=O)N1CCN(c2nc(=O)n3c4nc(c(F)cc24)-c2c(F)cccc2SCCCC(=O)Nc2ccnc(C(C)C)c2-3)[C@H](C)C1 ; C=CC(=O)N1CCN(c2nc(=O)n3c4nc(c(F)cc24)-c2c(F)cccc2SCCCCOc2ncnc(C(C)C)c2-3)[C@H]2C[C@H]21 ; C=CC(=O)N1CCN(c2nc(=O)n3c4nc(c(F)cc24)-c2c(F)cccc2OCCCCOc2ncnc(C(C)C)c2-3)[C@@H]2C[C@@H]21 ; C=CC(=O)N1CCN(c2nc(=O)n3c4nc(c(Cl)cc24)-c2c(F)cccc2OCCSc2ccnc(C(C)C)c2-3)CC1]

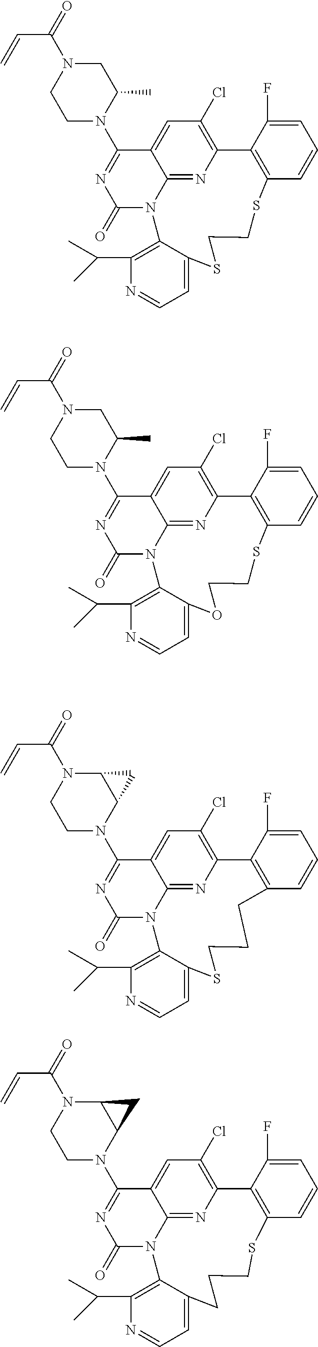 [SMILES: C=CC(=O)N1CCN(c2nc(=O)n3c4nc(c(Cl)cc24)-c2c(F)cccc2SCCSc2ccnc(C(C)C)c2-3)[C@@H](C)C1 ; C=CC(=O)N1CCN(c2nc(=O)n3c4nc(c(Cl)cc24)-c2c(F)cccc2SCCOc2ccnc(C(C)C)c2-3)[C@H](C)C1 ; C=CC(=O)N1CCN(c2nc(=O)n3c4nc(c(Cl)cc24)-c2c(F)cccc2CCCSc2ccnc(C(C)C)c2-3)[C@H]2C[C@H]21 ; C=CC(=O)N1CCN(c2nc(=O)n3c4nc(c(Cl)cc24)-c2c(F)cccc2SCCCc2ccnc(C(C)C)c2-3)[C@@H]2C[C@@H]21]

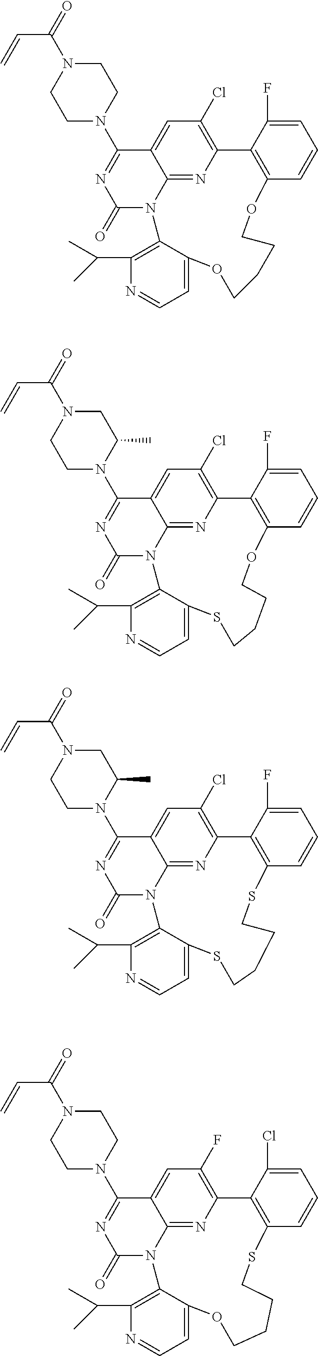 [SMILES: C=CC(=O)N1CCN(c2nc(=O)n3c4nc(c(Cl)cc24)-c2c(F)cccc2OCCCCOc2ccnc(C(C)C)c2-3)CC1 ; C=CC(=O)N1CCN(c2nc(=O)n3c4nc(c(Cl)cc24)-c2c(F)cccc2OCCCCSc2ccnc(C(C)C)c2-3)[C@@H](C)C1 ; C=CC(=O)N1CCN(c2nc(=O)n3c4nc(c(Cl)cc24)-c2c(F)cccc2SCCCCSc2ccnc(C(C)C)c2-3)[C@H](C)C1 ; C=CC(=O)N1CCN(c2nc(=O)n3c4nc(c(F)cc24)-c2c(Cl)cccc2SCCCCOc2ccnc(C(C)C)c2-3)CC1]

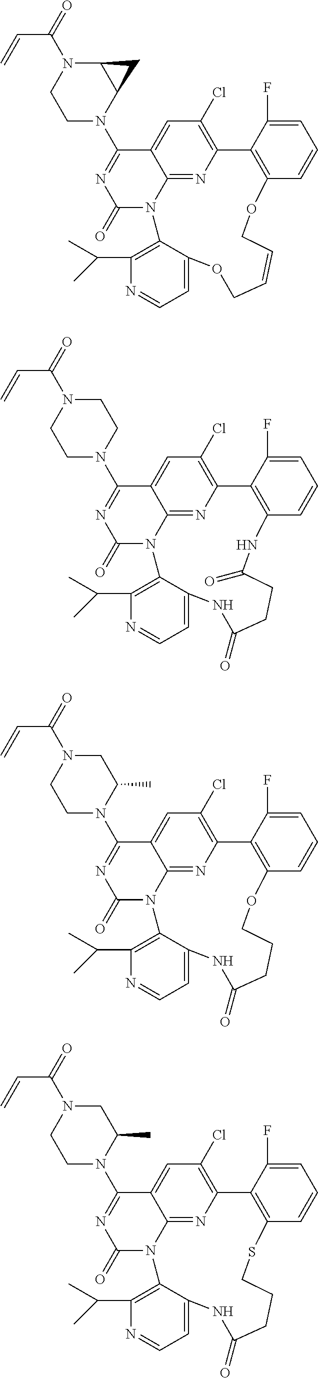 [SMILES: C=CC(=O)N1CCN(c2nc(=O)n3c4nc(c(Cl)cc24)-c2c(F)cccc2OC/C=C\COc2ccnc(C(C)C)c2-3)[C@@H]2C[C@@H]21 ; C=CC(=O)N1CCN(c2nc(=O)n3c4nc(c(Cl)cc24)-c2c(F)cccc2NC(=O)CCC(=O)Nc2ccnc(C(C)C)c2-3)CC1 ; C=CC(=O)N1CCN(c2nc(=O)n3c4nc(c(Cl)cc24)-c2c(F)cccc2OCCCC(=O)Nc2ccnc(C(C)C)c2-3)[C@@H](C)C1 ; C=CC(=O)N1CCN(c2nc(=O)n3c4nc(c(Cl)cc24)-c2c(F)cccc2SCCCC(=O)Nc2ccnc(C(C)C)c2-3)[C@H](C)C1]

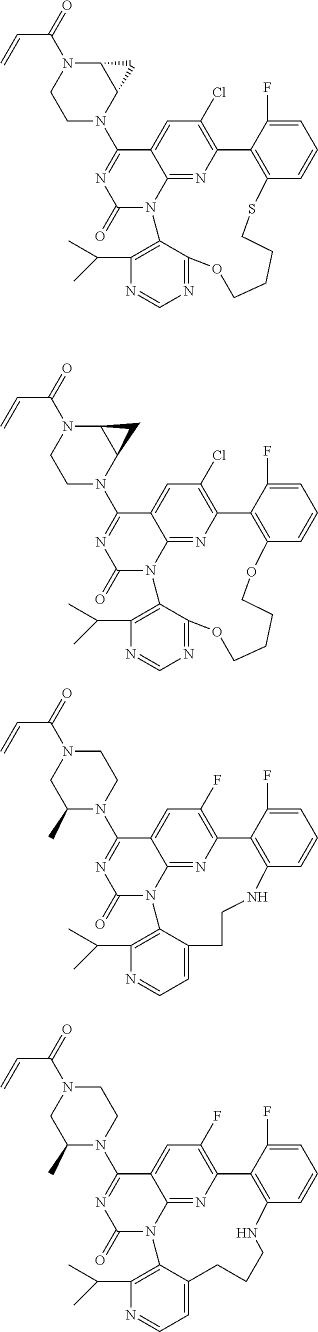 [SMILES: C=CC(=O)N1CCN(c2nc(=O)n3c4nc(c(Cl)cc24)-c2c(F)cccc2SCCCCOc2ncnc(C(C)C)c2-3)[C@H]2C[C@H]21 ; C=CC(=O)N1CCN(c2nc(=O)n3c4nc(c(Cl)cc24)-c2c(F)cccc2OCCCCOc2ncnc(C(C)C)c2-3)[C@@H]2C[C@@H]21 ; C=CC(=O)N1CCN(c2nc(=O)n3c4nc(c(F)cc24)-c2c(F)cccc2NCCc2ccnc(C(C)C)c2-3)[C@@H](C)C1 ; C=CC(=O)N1CCN(c2nc(=O)n3c4nc(c(F)cc24)-c2c(F)cccc2NCCCc2ccnc(C(C)C)c2-3)[C@@H](C)C1]

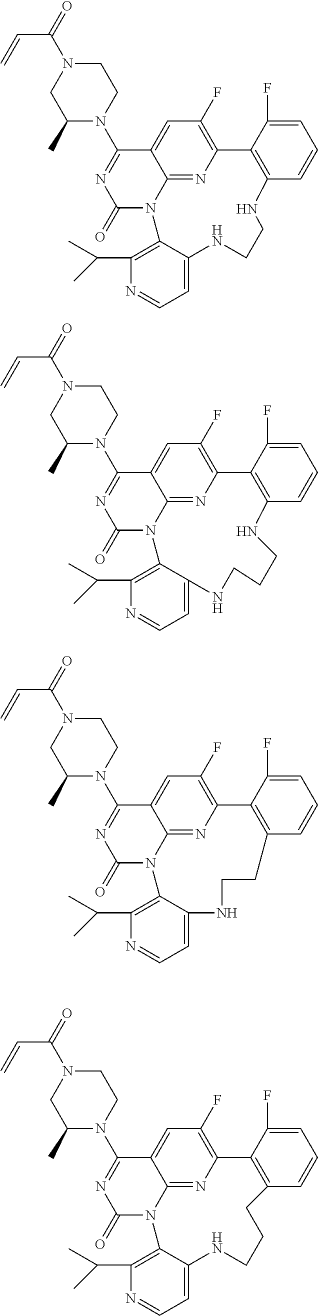 [SMILES: C=CC(=O)N1CCN(c2nc(=O)n3c4nc(c(F)cc24)-c2c(F)cccc2NCCNc2ccnc(C(C)C)c2-3)[C@@H](C)C1 ; C=CC(=O)N1CCN(c2nc(=O)n3c4nc(c(F)cc24)-c2c(F)cccc2NCCCNc2ccnc(C(C)C)c2-3)[C@@H](C)C1 ; C=CC(=O)N1CCN(c2nc(=O)n3c4nc(c(F)cc24)-c2c(F)cccc2CCNc2ccnc(C(C)C)c2-3)[C@@H](C)C1 ; C=CC(=O)N1CCN(c2nc(=O)n3c4nc(c(F)cc24)-c2c(F)cccc2CCCNc2ccnc(C(C)C)c2-3)[C@@H](C)C1]

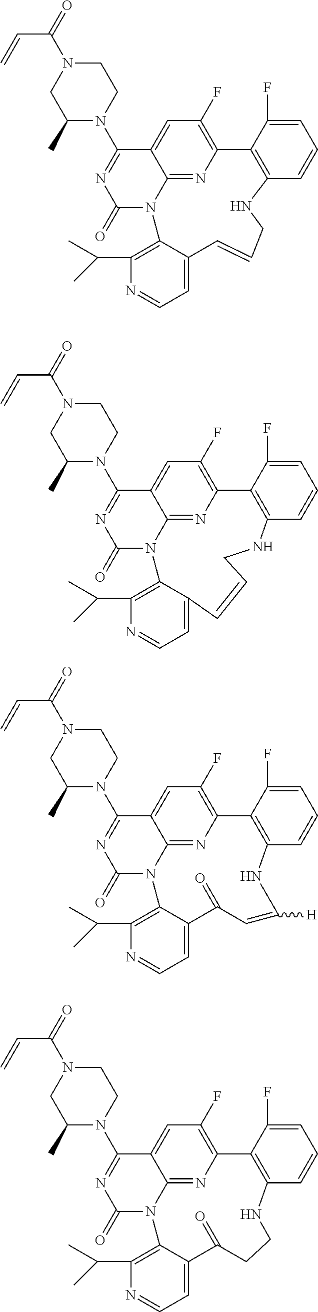 [SMILES: C=CC(=O)N1CCN(c2nc(=O)n3c4nc(c(F)cc24)-c2c(F)cccc2NC/C=C/c2ccnc(C(C)C)c2-3)[C@@H](C)C1 ; C=CC(=O)N1CCN(c2nc(=O)n3c4nc(c(F)cc24)-c2c(F)cccc2NC/C=C\c2ccnc(C(C)C)c2-3)[C@@H](C)C1 ; C=CC(=O)N1CCN(c2nc(=O)n3c4nc(c(F)cc24)-c2c(F)cccc2NC=CC(=O)c2ccnc(C(C)C)c2-3)[C@@H](C)C1 ; C=CC(=O)N1CCN(c2nc(=O)n3c4nc(c(F)cc24)-c2c(F)cccc2NCCC(=O)c2ccnc(C(C)C)c2-3)[C@@H](C)C1]

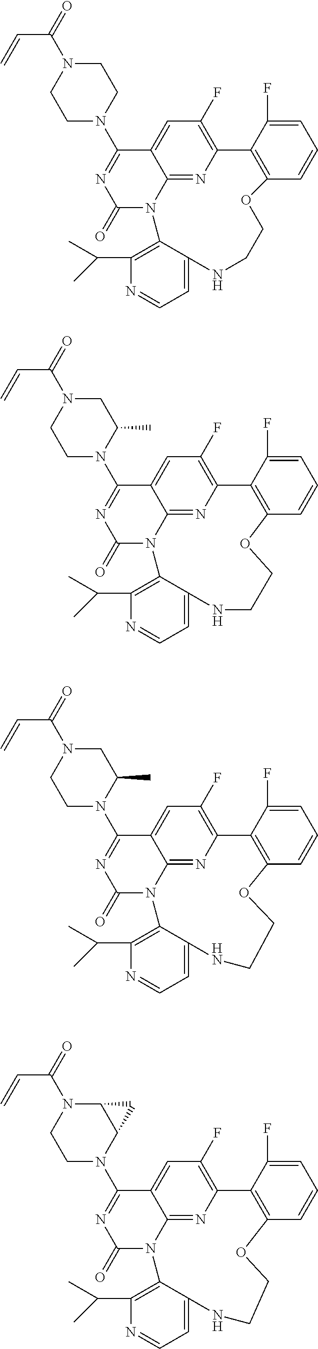 [SMILES: C=CC(=O)N1CCN(c2nc(=O)n3c4nc(c(F)cc24)-c2c(F)cccc2OCCNc2ccnc(C(C)C)c2-3)CC1 ; C=CC(=O)N1CCN(c2nc(=O)n3c4nc(c(F)cc24)-c2c(F)cccc2OCCNc2ccnc(C(C)C)c2-3)[C@@H](C)C1 ; C=CC(=O)N1CCN(c2nc(=O)n3c4nc(c(F)cc24)-c2c(F)cccc2OCCNc2ccnc(C(C)C)c2-3)[C@H](C)C1 ; C=CC(=O)N1CCN(c2nc(=O)n3c4nc(c(F)cc24)-c2c(F)cccc2OCCNc2ccnc(C(C)C)c2-3)[C@H]2C[C@H]21]

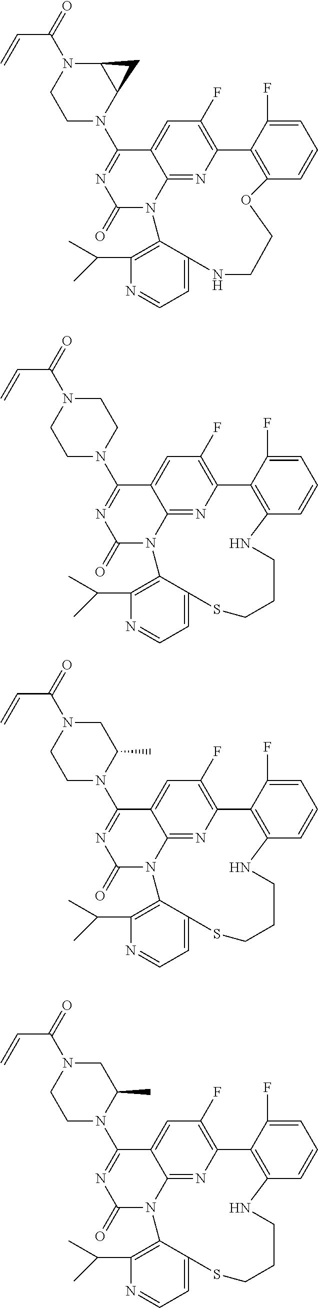 [SMILES: C=CC(=O)N1CCN(c2nc(=O)n3c4nc(c(F)cc24)-c2c(F)cccc2OCCNc2ccnc(C(C)C)c2-3)[C@@H]2C[C@@H]21 ; C=CC(=O)N1CCN(c2nc(=O)n3c4nc(c(F)cc24)-c2c(F)cccc2NCCCSc2ccnc(C(C)C)c2-3)CC1 ; C=CC(=O)N1CCN(c2nc(=O)n3c4nc(c(F)cc24)-c2c(F)cccc2NCCCSc2ccnc(C(C)C)c2-3)[C@@H](C)C1 ; C=CC(=O)N1CCN(c2nc(=O)n3c4nc(c(F)cc24)-c2c(F)cccc2NCCCSc2ccnc(C(C)C)c2-3)[C@H](C)C1]

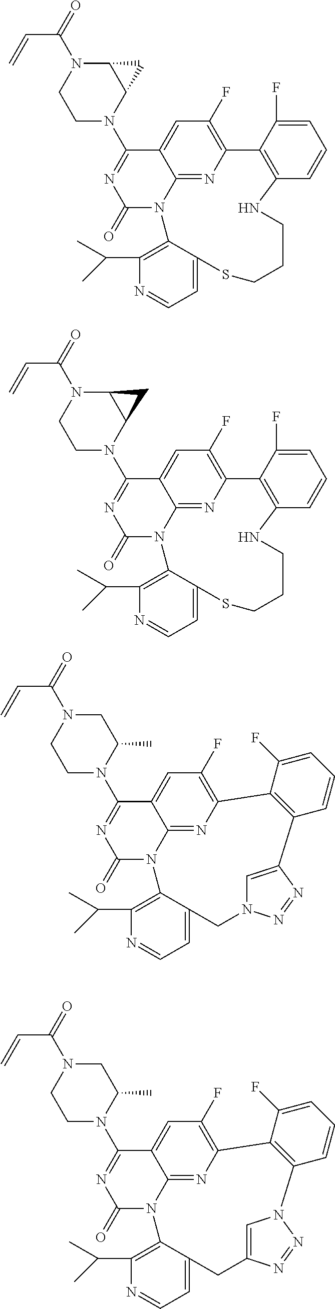 [SMILES: C=CC(=O)N1CCN(c2nc(=O)n3c4nc(c(F)cc24)-c2c(F)cccc2NCCCSc2ccnc(C(C)C)c2-3)[C@H]2C[C@H]21 ; C=CC(=O)N1CCN(c2nc(=O)n3c4nc(c(F)cc24)-c2c(F)cccc2NCCCSc2ccnc(C(C)C)c2-3)[C@@H]2C[C@@H]21 ; C=CC(=O)N1CCN(c2nc(=O)n3c4nc(c(F)cc24)-c2c(F)cccc2-c2cn(nn2)Cc2ccnc(C(C)C)c2-3)[C@@H](C)C1 ; C=CC(=O)N1CCN(c2nc(=O)n3c4nc(c(F)cc24)-c2c(F)cccc2-n2cc(nn2)Cc2ccnc(C(C)C)c2-3)[C@@H](C)C1]

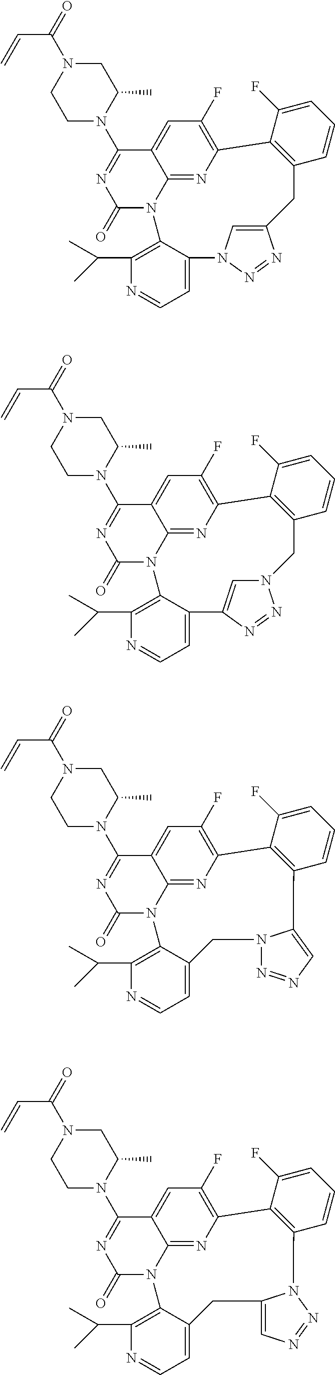 [SMILES: C=CC(=O)N1CCN(c2nc(=O)n3c4nc(c(F)cc24)-c2c(F)cccc2Cc2cn(nn2)-c2ccnc(C(C)C)c2-3)[C@@H](C)C1 ; C=CC(=O)N1CCN(c2nc(=O)n3c4nc(c(F)cc24)-c2c(F)cccc2Cn2cc(nn2)-c2ccnc(C(C)C)c2-3)[C@@H](C)C1 ; C=CC(=O)N1CCN(c2nc(=O)n3c4nc(c(F)cc24)-c2c(F)cccc2-c2cnnn2Cc2ccnc(C(C)C)c2-3)[C@@H](C)C1 ; C=CC(=O)N1CCN(c2nc(=O)n3c4nc(c(F)cc24)-c2c(F)cccc2-n2nncc2Cc2ccnc(C(C)C)c2-3)[C@@H](C)C1]

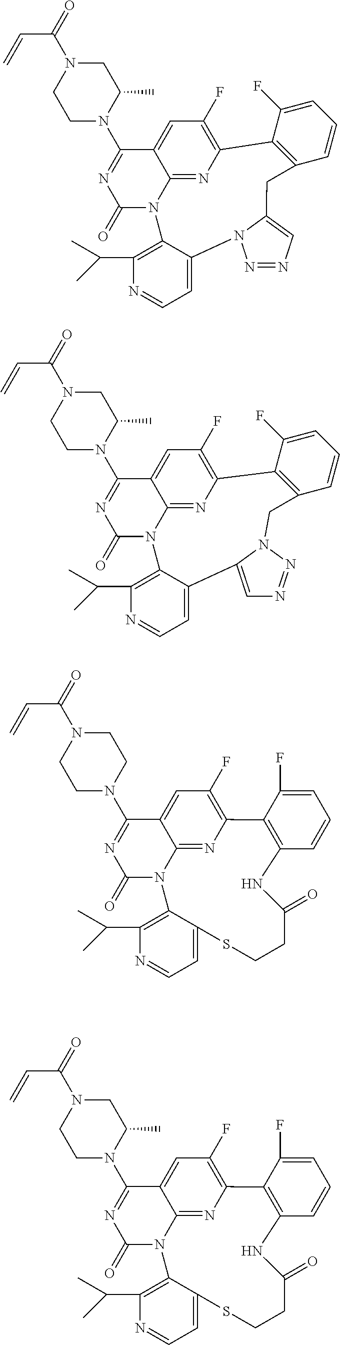 [SMILES: C=CC(=O)N1CCN(c2nc(=O)n3c4nc(c(F)cc24)-c2c(F)cccc2Cc2cnnn2-c2ccnc(C(C)C)c2-3)[C@@H](C)C1 ; C=CC(=O)N1CCN(c2nc(=O)n3c4nc(c(F)cc24)-c2c(F)cccc2Cn2nncc2-c2ccnc(C(C)C)c2-3)[C@@H](C)C1 ; C=CC(=O)N1CCN(c2nc(=O)n3c4nc(c(F)cc24)-c2c(F)cccc2NC(=O)CCSc2ccnc(C(C)C)c2-3)CC1 ; C=CC(=O)N1CCN(c2nc(=O)n3c4nc(c(F)cc24)-c2c(F)cccc2NC(=O)CCSc2ccnc(C(C)C)c2-3)[C@@H](C)C1]

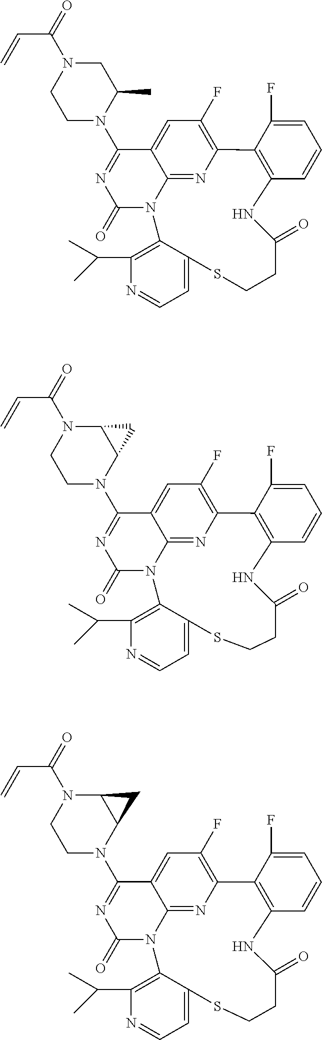 [SMILES: C=CC(=O)N1CCN(c2nc(=O)n3c4nc(c(F)cc24)-c2c(F)cccc2NC(=O)CCSc2ccnc(C(C)C)c2-3)[C@H](C)C1 ; C=CC(=O)N1CCN(c2nc(=O)n3c4nc(c(F)cc24)-c2c(F)cccc2NC(=O)CCSc2ccnc(C(C)C)c2-3)[C@H]2C[C@H]21 ; C=CC(=O)N1CCN(c2nc(=O)n3c4nc(c(F)cc24)-c2c(F)cccc2NC(=O)CCSc2ccnc(C(C)C)c2-3)[C@@H]2C[C@@H]21]

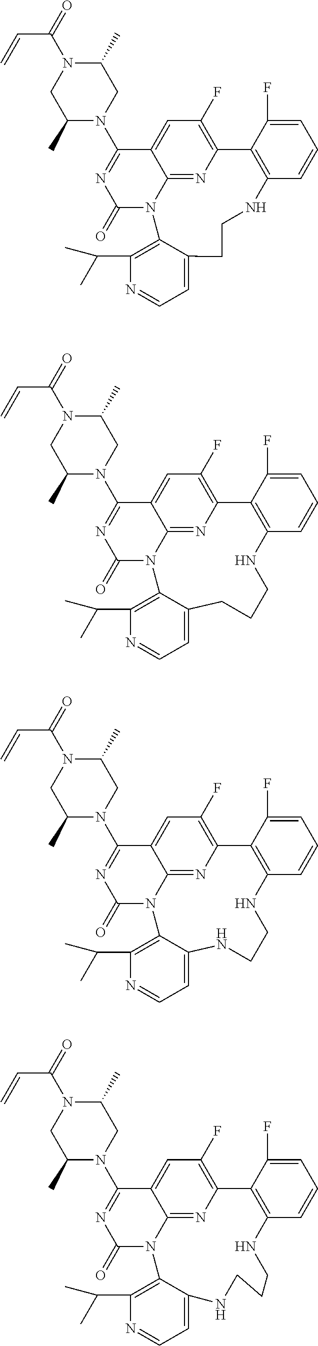 [SMILES: C=CC(=O)N1C[C@H](C)N(c2nc(=O)n3c4nc(c(F)cc24)-c2c(F)cccc2NCCc2ccnc(C(C)C)c2-3)C[C@H]1C ; C=CC(=O)N1C[C@H](C)N(c2nc(=O)n3c4nc(c(F)cc24)-c2c(F)cccc2NCCCc2ccnc(C(C)C)c2-3)C[C@H]1C ; C=CC(=O)N1C[C@H](C)N(c2nc(=O)n3c4nc(c(F)cc24)-c2c(F)cccc2NCCNc2ccnc(C(C)C)c2-3)C[C@H]1C ; C=CC(=O)N1C[C@H](C)N(c2nc(=O)n3c4nc(c(F)cc24)-c2c(F)cccc2NCCCNc2ccnc(C(C)C)c2-3)C[C@H]1C]

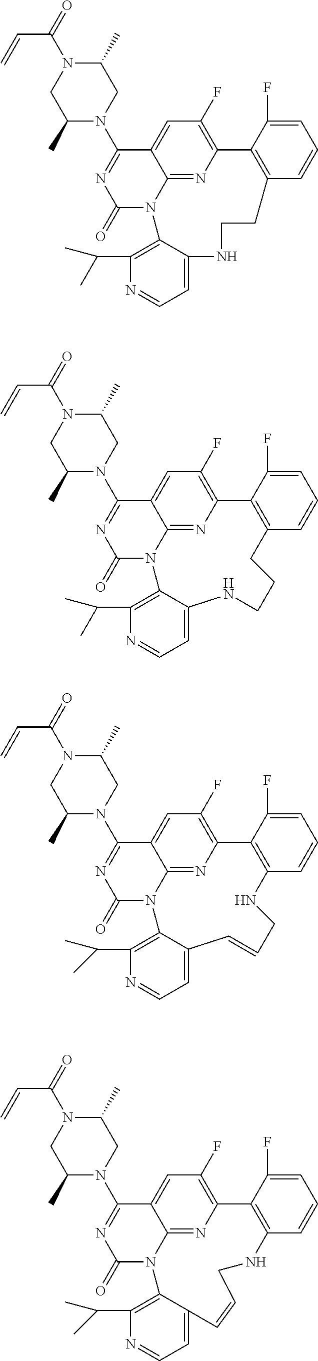 [SMILES: C=CC(=O)N1C[C@H](C)N(c2nc(=O)n3c4nc(c(F)cc24)-c2c(F)cccc2CCNc2ccnc(C(C)C)c2-3)C[C@H]1C ; C=CC(=O)N1C[C@H](C)N(c2nc(=O)n3c4nc(c(F)cc24)-c2c(F)cccc2CCCNc2ccnc(C(C)C)c2-3)C[C@H]1C ; C=CC(=O)N1C[C@H](C)N(c2nc(=O)n3c4nc(c(F)cc24)-c2c(F)cccc2NC/C=C/c2ccnc(C(C)C)c2-3)C[C@H]1C ; C=CC(=O)N1C[C@H](C)N(c2nc(=O)n3c4nc(c(F)cc24)-c2c(F)cccc2NC/C=C\c2ccnc(C(C)C)c2-3)C[C@H]1C]

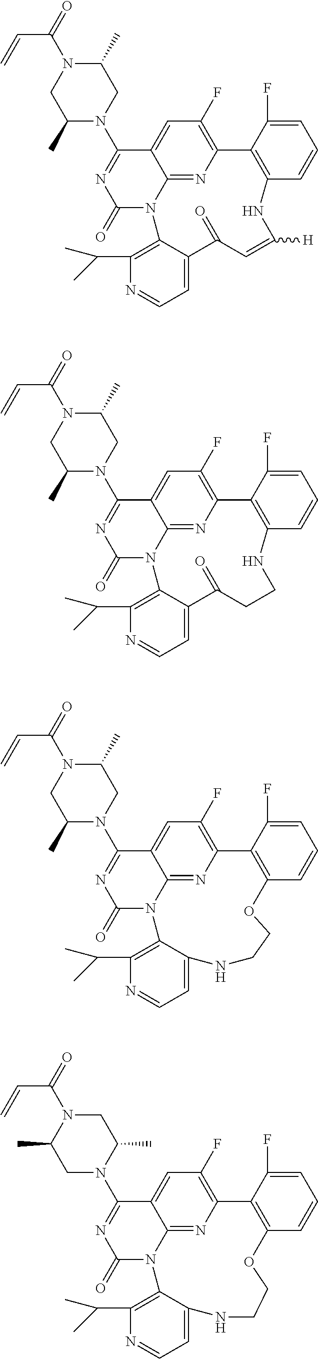 [SMILES: C=CC(=O)N1C[C@H](C)N(c2nc(=O)n3c4nc(c(F)cc24)-c2c(F)cccc2NC=CC(=O)c2ccnc(C(C)C)c2-3)C[C@H]1C ; C=CC(=O)N1C[C@H](C)N(c2nc(=O)n3c4nc(c(F)cc24)-c2c(F)cccc2NCCC(=O)c2ccnc(C(C)C)c2-3)C[C@H]1C ; C=CC(=O)N1C[C@H](C)N(c2nc(=O)n3c4nc(c(F)cc24)-c2c(F)cccc2OCCNc2ccnc(C(C)C)c2-3)C[C@H]1C ; C=CC(=O)N1C[C@H](C)N(c2nc(=O)n3c4nc(c(F)cc24)-c2c(F)cccc2OCCNc2ccnc(C(C)C)c2-3)C[C@H]1C]

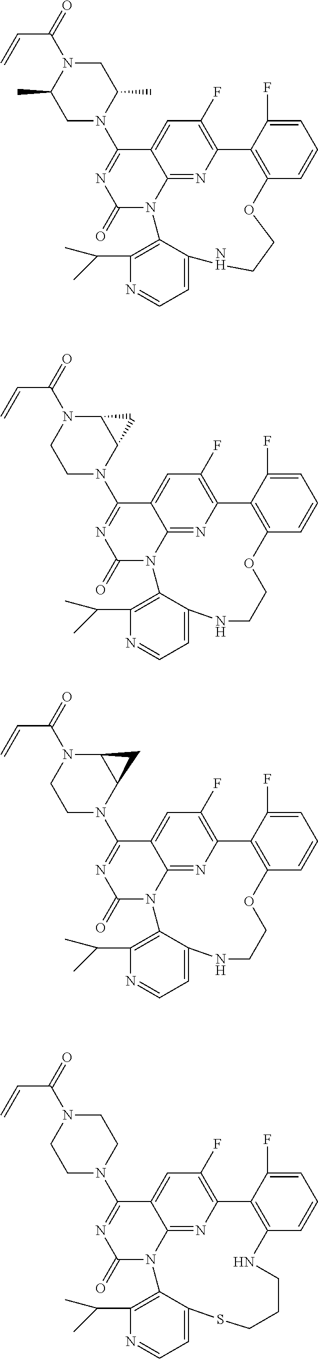 [SMILES: C=CC(=O)N1C[C@H](C)N(c2nc(=O)n3c4nc(c(F)cc24)-c2c(F)cccc2OCCNc2ccnc(C(C)C)c2-3)C[C@H]1C ; C=CC(=O)N1CCN(c2nc(=O)n3c4nc(c(F)cc24)-c2c(F)cccc2OCCNc2ccnc(C(C)C)c2-3)[C@H]2C[C@H]21 ; C=CC(=O)N1CCN(c2nc(=O)n3c4nc(c(F)cc24)-c2c(F)cccc2OCCNc2ccnc(C(C)C)c2-3)[C@@H]2C[C@@H]21 ; C=CC(=O)N1CCN(c2nc(=O)n3c4nc(c(F)cc24)-c2c(F)cccc2NCCCSc2ccnc(C(C)C)c2-3)CC1]

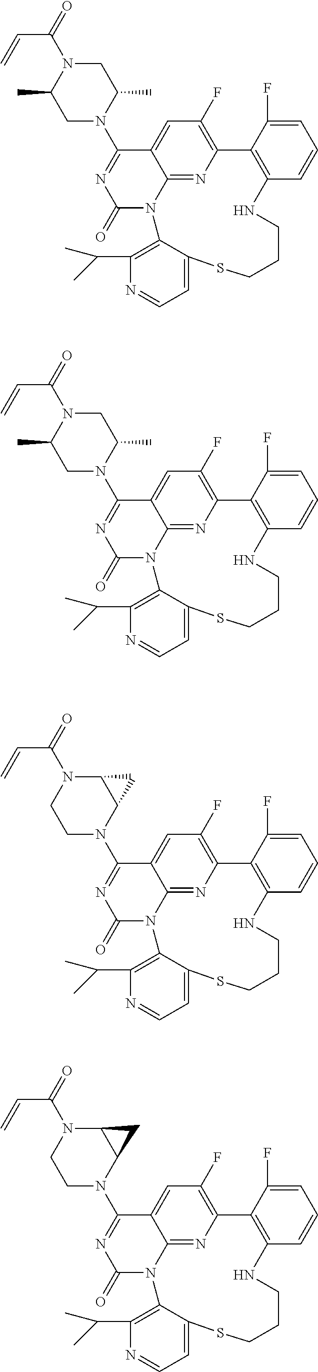 [SMILES: C=CC(=O)N1C[C@H](C)N(c2nc(=O)n3c4nc(c(F)cc24)-c2c(F)cccc2NCCCSc2ccnc(C(C)C)c2-3)C[C@H]1C ; C=CC(=O)N1C[C@H](C)N(c2nc(=O)n3c4nc(c(F)cc24)-c2c(F)cccc2NCCCSc2ccnc(C(C)C)c2-3)C[C@H]1C ; C=CC(=O)N1CCN(c2nc(=O)n3c4nc(c(F)cc24)-c2c(F)cccc2NCCCSc2ccnc(C(C)C)c2-3)[C@H]2C[C@H]21 ; C=CC(=O)N1CCN(c2nc(=O)n3c4nc(c(F)cc24)-c2c(F)cccc2NCCCSc2ccnc(C(C)C)c2-3)[C@@H]2C[C@@H]21]

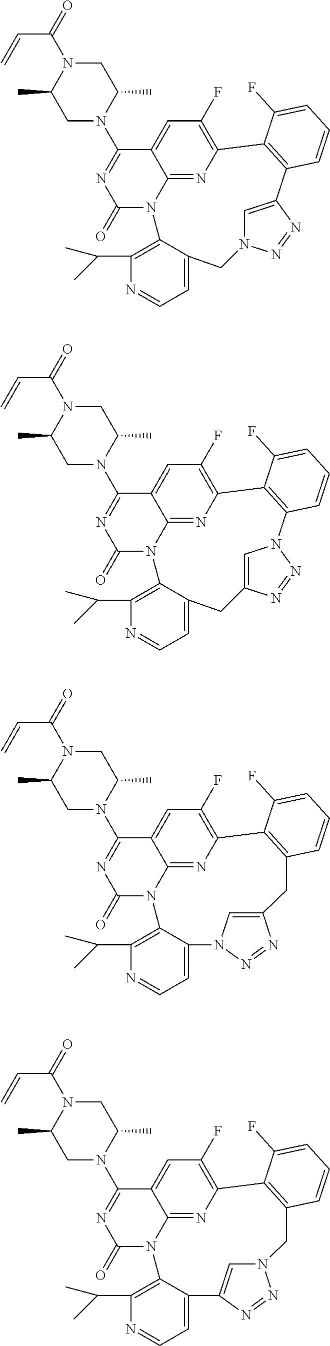 [SMILES: C=CC(=O)N1C[C@H](C)N(c2nc(=O)n3c4nc(c(F)cc24)-c2c(F)cccc2-c2cn(nn2)Cc2ccnc(C(C)C)c2-3)C[C@H]1C ; C=CC(=O)N1C[C@H](C)N(c2nc(=O)n3c4nc(c(F)cc24)-c2c(F)cccc2-n2cc(nn2)Cc2ccnc(C(C)C)c2-3)C[C@H]1C ; C=CC(=O)N1C[C@H](C)N(c2nc(=O)n3c4nc(c(F)cc24)-c2c(F)cccc2Cc2cn(nn2)-c2ccnc(C(C)C)c2-3)C[C@H]1C ; C=CC(=O)N1C[C@H](C)N(c2nc(=O)n3c4nc(c(F)cc24)-c2c(F)cccc2Cn2cc(nn2)-c2ccnc(C(C)C)c2-3)C[C@H]1C]

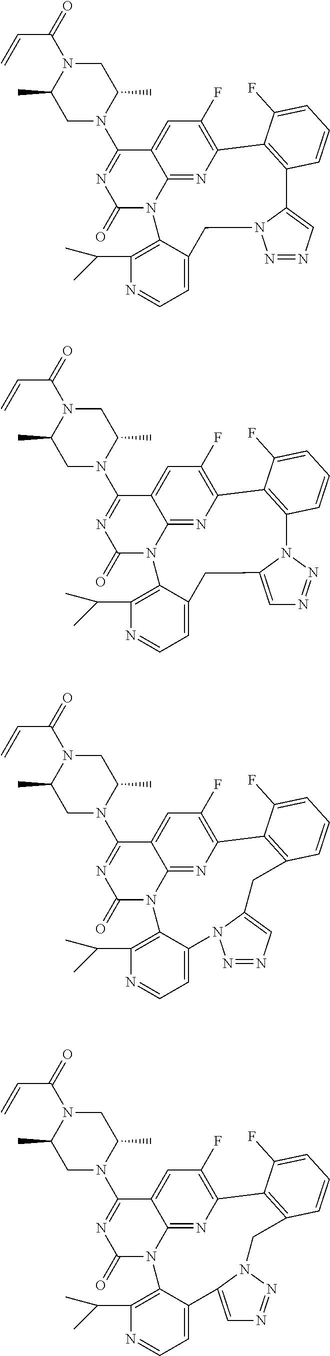 [SMILES: C=CC(=O)N1C[C@H](C)N(c2nc(=O)n3c4nc(c(F)cc24)-c2c(F)cccc2-c2cnnn2Cc2ccnc(C(C)C)c2-3)C[C@H]1C ; C=CC(=O)N1C[C@H](C)N(c2nc(=O)n3c4nc(c(F)cc24)-c2c(F)cccc2-n2nncc2Cc2ccnc(C(C)C)c2-3)C[C@H]1C ; C=CC(=O)N1C[C@H](C)N(c2nc(=O)n3c4nc(c(F)cc24)-c2c(F)cccc2Cc2cnnn2-c2ccnc(C(C)C)c2-3)C[C@H]1C ; C=CC(=O)N1C[C@H](C)N(c2nc(=O)n3c4nc(c(F)cc24)-c2c(F)cccc2Cn2nncc2-c2ccnc(C(C)C)c2-3)C[C@H]1C]

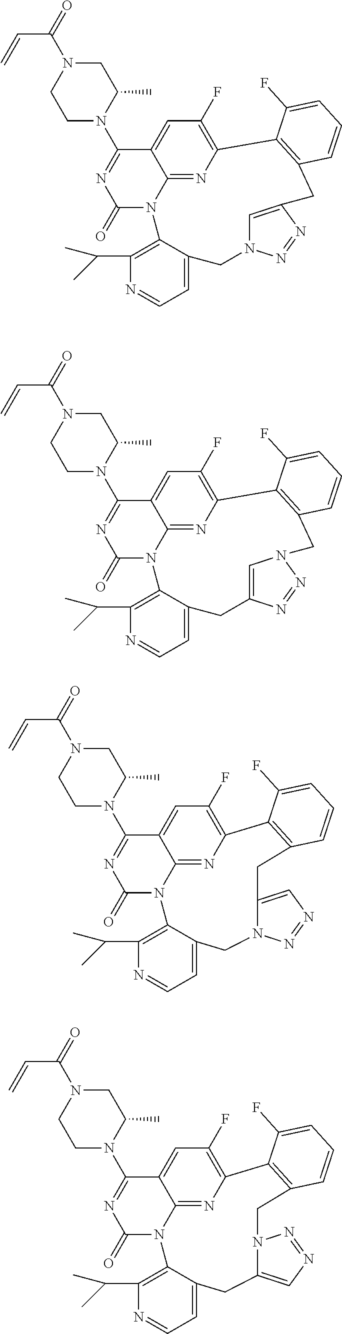 [SMILES: C=CC(=O)N1CCN(c2nc(=O)n3c4nc(c(F)cc24)-c2c(F)cccc2Cc2cn(nn2)Cc2ccnc(C(C)C)c2-3)[C@@H](C)C1 ; C=CC(=O)N1CCN(c2nc(=O)n3c4nc(c(F)cc24)-c2c(F)cccc2Cn2cc(nn2)Cc2ccnc(C(C)C)c2-3)[C@@H](C)C1 ; C=CC(=O)N1CCN(c2nc(=O)n3c4nc(c(F)cc24)-c2c(F)cccc2Cc2cnnn2Cc2ccnc(C(C)C)c2-3)[C@@H](C)C1 ; C=CC(=O)N1CCN(c2nc(=O)n3c4nc(c(F)cc24)-c2c(F)cccc2Cn2nncc2Cc2ccnc(C(C)C)c2-3)[C@@H](C)C1]

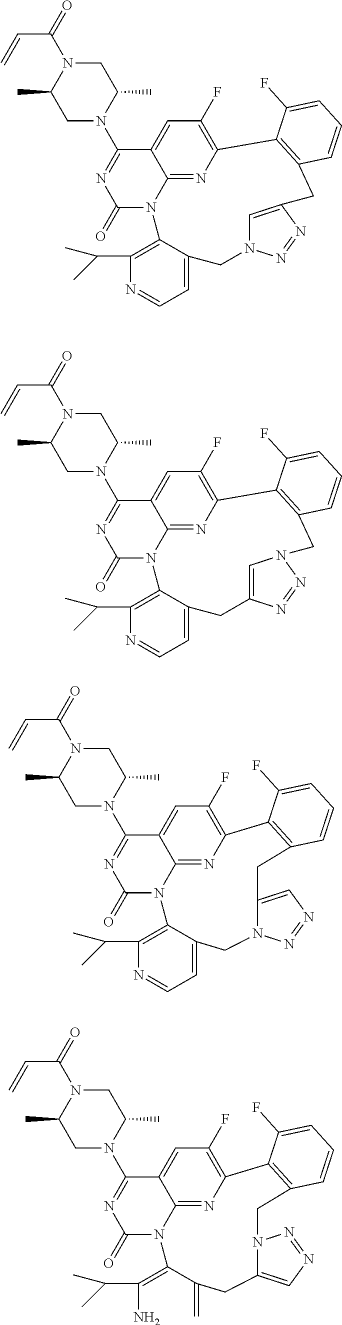 [SMILES: C=CC(=O)N1C[C@H](C)N(c2nc(=O)n3c4nc(c(F)cc24)-c2c(F)cccc2Cc2cn(nn2)Cc2ccnc(C(C)C)c2-3)C[C@H]1C ; C=CC(=O)N1C[C@H](C)N(c2nc(=O)n3c4nc(c(F)cc24)-c2c(F)cccc2Cn2cc(nn2)Cc2ccnc(C(C)C)c2-3)C[C@H]1C ; C=CC(=O)N1C[C@H](C)N(c2nc(=O)n3c4nc(c(F)cc24)-c2c(F)cccc2Cc2cnnn2Cc2ccnc(C(C)C)c2-3)C[C@H]1C ; C=CC(=O)N1C[C@H](C)N(c2nc(=O)n3c4nc(c(F)cc24)-c2c(F)cccc2Cn2nncc2CC(=C)/C3=C(\N)C(C)C)C[C@H]1C]

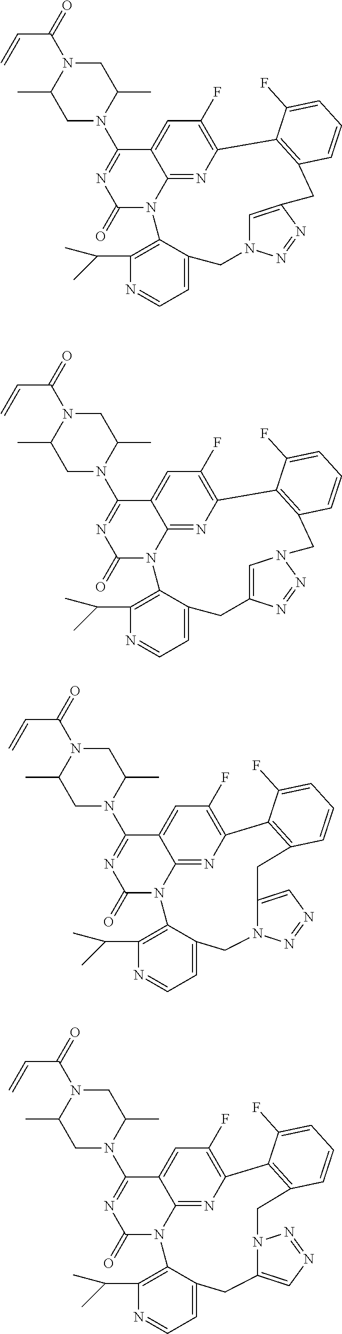 [SMILES: C=CC(=O)N1CC(C)N(c2nc(=O)n3c4nc(c(F)cc24)-c2c(F)cccc2Cc2cn(nn2)Cc2ccnc(C(C)C)c2-3)CC1C ; C=CC(=O)N1CC(C)N(c2nc(=O)n3c4nc(c(F)cc24)-c2c(F)cccc2Cn2cc(nn2)Cc2ccnc(C(C)C)c2-3)CC1C ; C=CC(=O)N1CC(C)N(c2nc(=O)n3c4nc(c(F)cc24)-c2c(F)cccc2Cc2cnnn2Cc2ccnc(C(C)C)c2-3)CC1C ; C=CC(=O)N1CC(C)N(c2nc(=O)n3c4nc(c(F)cc24)-c2c(F)cccc2Cn2nncc2Cc2ccnc(C(C)C)c2-3)CC1C]

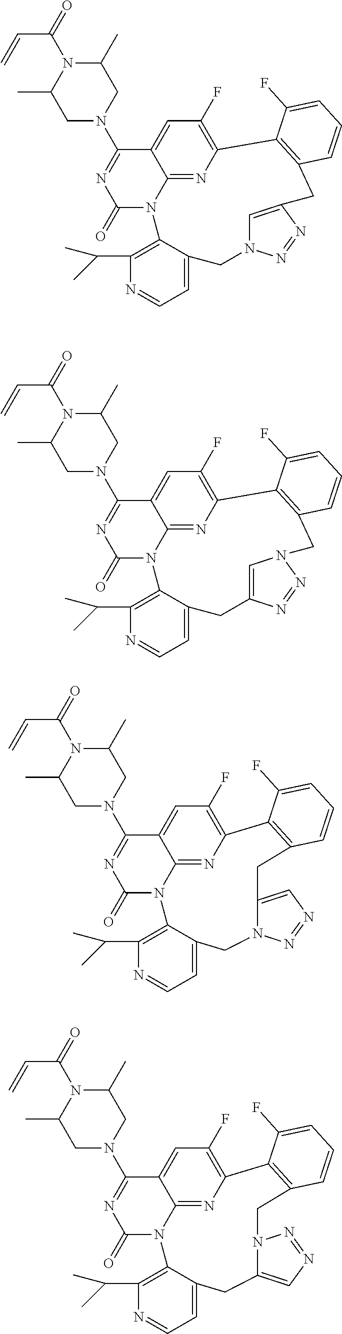 [SMILES: C=CC(=O)N1C(C)CN(c2nc(=O)n3c4nc(c(F)cc24)-c2c(F)cccc2Cc2cn(nn2)Cc2ccnc(C(C)C)c2-3)CC1C ; C=CC(=O)N1C(C)CN(c2nc(=O)n3c4nc(c(F)cc24)-c2c(F)cccc2Cn2cc(nn2)Cc2ccnc(C(C)C)c2-3)CC1C ; C=CC(=O)N1C(C)CN(c2nc(=O)n3c4nc(c(F)cc24)-c2c(F)cccc2Cc2cnnn2Cc2ccnc(C(C)C)c2-3)CC1C ; C=CC(=O)N1C(C)CN(c2nc(=O)n3c4nc(c(F)cc24)-c2c(F)cccc2Cn2nncc2Cc2ccnc(C(C)C)c2-3)CC1C]

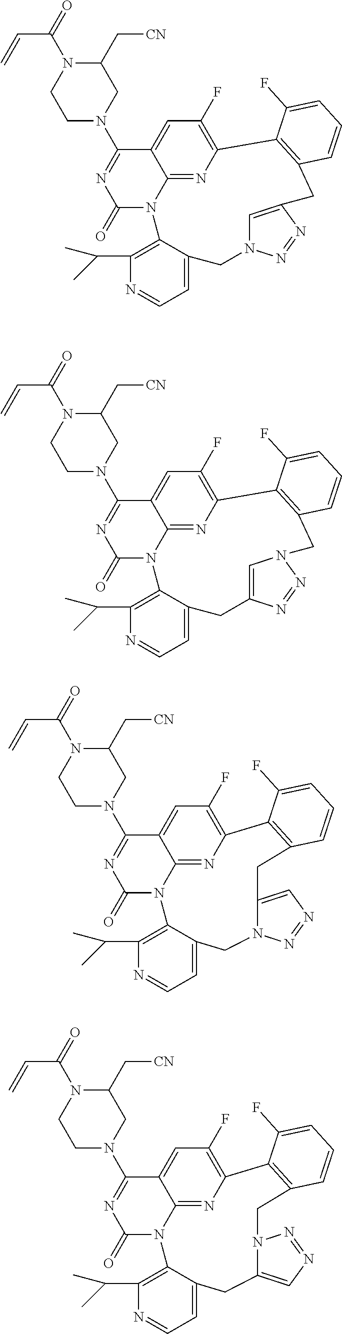 [SMILES: C=CC(=O)N1CCN(c2nc(=O)n3c4nc(c(F)cc24)-c2c(F)cccc2Cc2cn(nn2)Cc2ccnc(C(C)C)c2-3)CC1CC#N ; C=CC(=O)N1CCN(c2nc(=O)n3c4nc(c(F)cc24)-c2c(F)cccc2Cn2cc(nn2)Cc2ccnc(C(C)C)c2-3)CC1CC#N ; C=CC(=O)N1CCN(c2nc(=O)n3c4nc(c(F)cc24)-c2c(F)cccc2Cc2cnnn2Cc2ccnc(C(C)C)c2-3)CC1CC#N ; C=CC(=O)N1CCN(c2nc(=O)n3c4nc(c(F)cc24)-c2c(F)cccc2Cn2nncc2Cc2ccnc(C(C)C)c2-3)CC1CC#N]

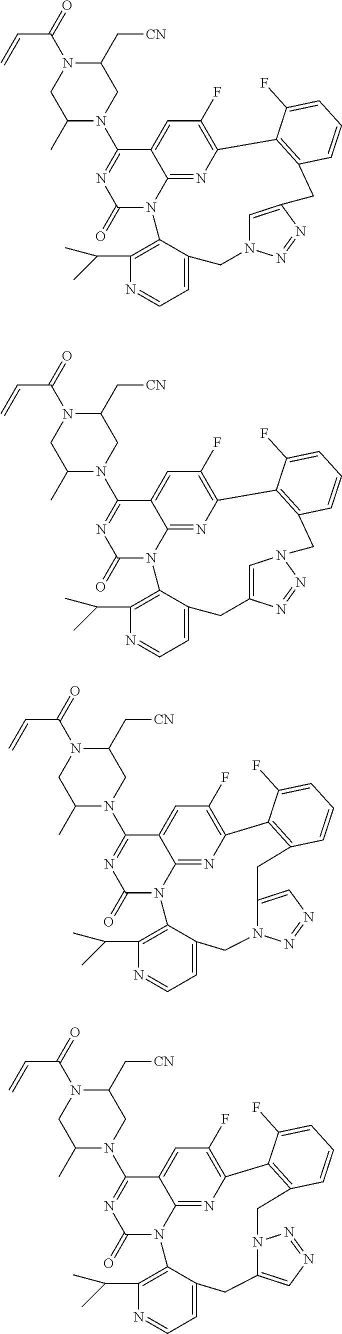 [SMILES: C=CC(=O)N1CC(C)N(c2nc(=O)n3c4nc(c(F)cc24)-c2c(F)cccc2Cc2cn(nn2)Cc2ccnc(C(C)C)c2-3)CC1CC#N ; C=CC(=O)N1CC(C)N(c2nc(=O)n3c4nc(c(F)cc24)-c2c(F)cccc2Cn2cc(nn2)Cc2ccnc(C(C)C)c2-3)CC1CC#N ; C=CC(=O)N1CC(C)N(c2nc(=O)n3c4nc(c(F)cc24)-c2c(F)cccc2Cc2cnnn2Cc2ccnc(C(C)C)c2-3)CC1CC#N ; C=CC(=O)N1CC(C)N(c2nc(=O)n3c4nc(c(F)cc24)-c2c(F)cccc2Cn2nncc2Cc2ccnc(C(C)C)c2-3)CC1CC#N]

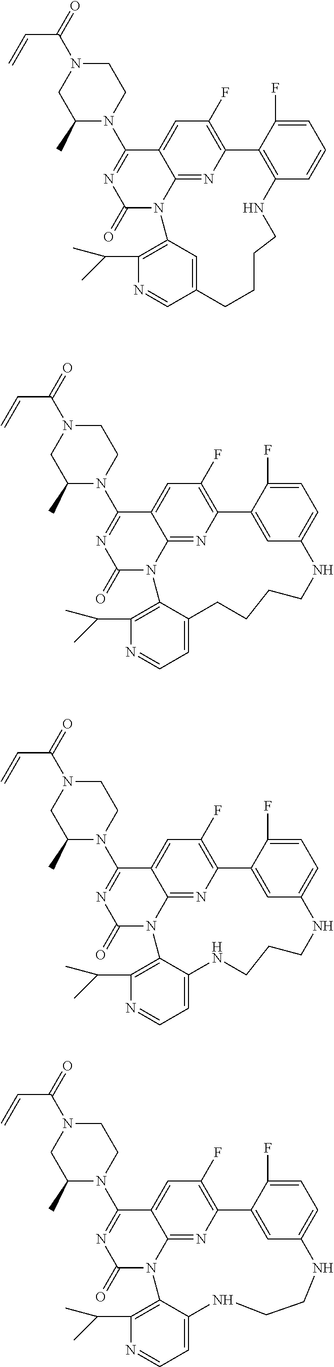 [SMILES: C=CC(=O)N1CCN(c2nc(=O)n3c4nc(c(F)cc24)-c2c(F)cccc2NCCCCc2cnc(C(C)C)c-3c2)[C@@H](C)C1 ; C=CC(=O)N1CCN(c2nc(=O)n3c4nc(c(F)cc24)-c2cc(ccc2F)NCCCCc2ccnc(C(C)C)c2-3)[C@@H](C)C1 ; C=CC(=O)N1CCN(c2nc(=O)n3c4nc(c(F)cc24)-c2cc(ccc2F)NCCCNc2ccnc(C(C)C)c2-3)[C@@H](C)C1 ; C=CC(=O)N1CCN(c2nc(=O)n3c4nc(c(F)cc24)-c2cc(ccc2F)NCCNc2ccnc(C(C)C)c2-3)[C@@H](C)C1]

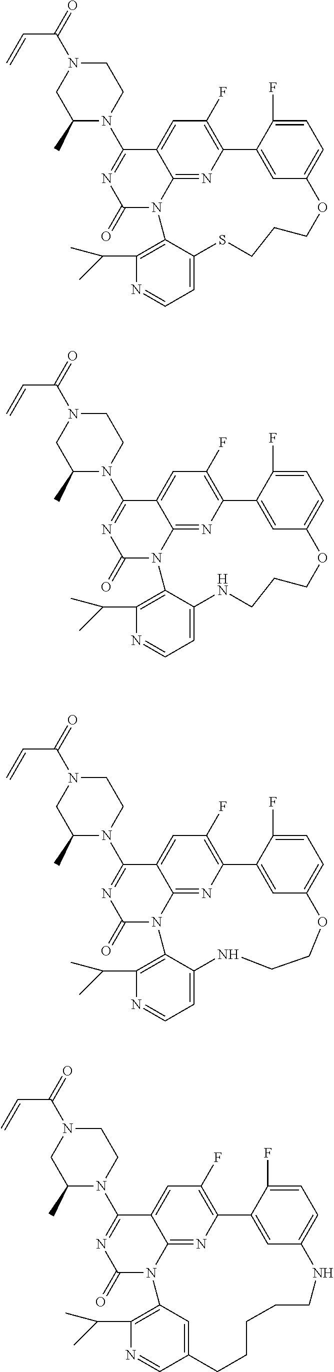 [SMILES: C=CC(=O)N1CCN(c2nc(=O)n3c4nc(c(F)cc24)-c2cc(ccc2F)OCCCSc2ccnc(C(C)C)c2-3)[C@@H](C)C1 ; C=CC(=O)N1CCN(c2nc(=O)n3c4nc(c(F)cc24)-c2cc(ccc2F)OCCCNc2ccnc(C(C)C)c2-3)[C@@H](C)C1 ; C=CC(=O)N1CCN(c2nc(=O)n3c4nc(c(F)cc24)-c2cc(ccc2F)OCCNc2ccnc(C(C)C)c2-3)[C@@H](C)C1 ; C=CC(=O)N1CCN(c2nc(=O)n3c4nc(c(F)cc24)-c2cc(ccc2F)NCCCCCc2cnc(C(C)C)c-3c2)[C@@H](C)C1]

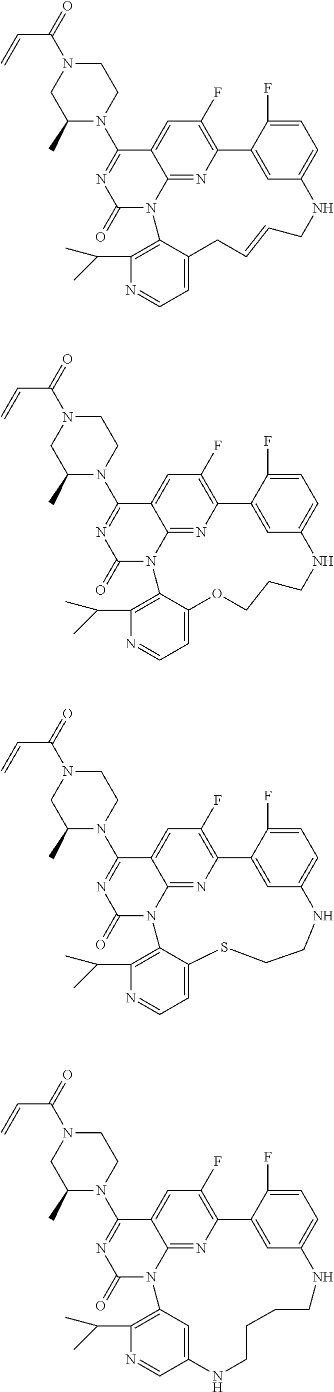 [SMILES: C=CC(=O)N1CCN(c2nc(=O)n3c4nc(c(F)cc24)-c2cc(ccc2F)NC/C=C/Cc2ccnc(C(C)C)c2-3)[C@@H](C)C1 ; C=CC(=O)N1CCN(c2nc(=O)n3c4nc(c(F)cc24)-c2cc(ccc2F)NCCCOc2ccnc(C(C)C)c2-3)[C@@H](C)C1 ; C=CC(=O)N1CCN(c2nc(=O)n3c4nc(c(F)cc24)-c2cc(ccc2F)NCCSc2ccnc(C(C)C)c2-3)[C@@H](C)C1 ; C=CC(=O)N1CCN(c2nc(=O)n3c4nc(c(F)cc24)-c2cc(ccc2F)NCCCCNc2cnc(C(C)C)c-3c2)[C@@H](C)C1]

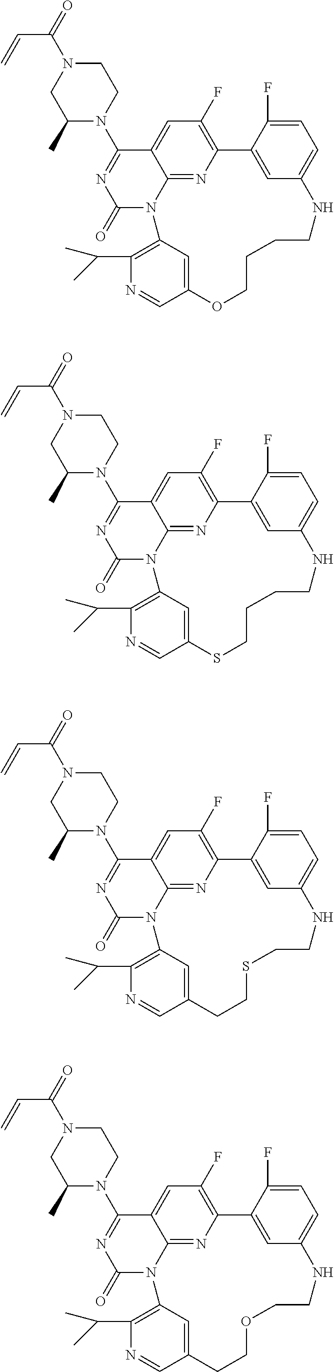 [SMILES: C=CC(=O)N1CCN(c2nc(=O)n3c4nc(c(F)cc24)-c2cc(ccc2F)NCCCCOc2cnc(C(C)C)c-3c2)[C@@H](C)C1 ; C=CC(=O)N1CCN(c2nc(=O)n3c4nc(c(F)cc24)-c2cc(ccc2F)NCCCCSc2cnc(C(C)C)c-3c2)[C@@H](C)C1 ; C=CC(=O)N1CCN(c2nc(=O)n3c4nc(c(F)cc24)-c2cc(ccc2F)NCCSCCc2cnc(C(C)C)c-3c2)[C@@H](C)C1 ; C=CC(=O)N1CCN(c2nc(=O)n3c4nc(c(F)cc24)-c2cc(ccc2F)NCCOCCc2cnc(C(C)C)c-3c2)[C@@H](C)C1]

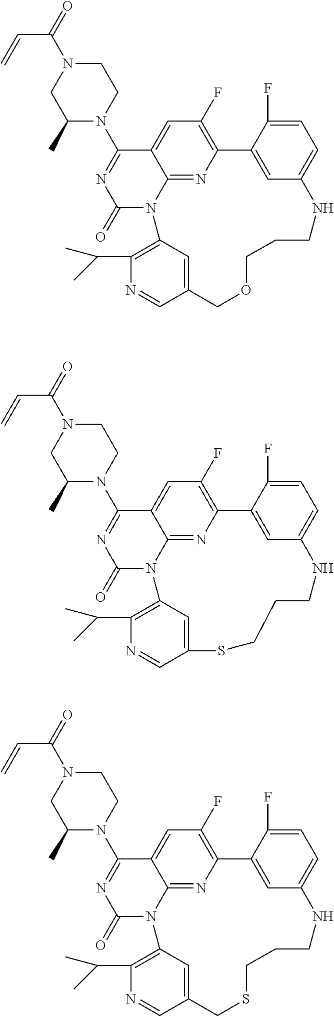 [SMILES: C=CC(=O)N1CCN(c2nc(=O)n3c4nc(c(F)cc24)-c2cc(ccc2F)NCCCOCc2cnc(C(C)C)c-3c2)[C@@H](C)C1 ; C=CC(=O)N1CCN(c2nc(=O)n3c4nc(c(F)cc24)-c2cc(ccc2F)NCCCSc2cnc(C(C)C)c-3c2)[C@@H](C)C1 ; C=CC(=O)N1CCN(c2nc(=O)n3c4nc(c(F)cc24)-c2cc(ccc2F)NCCCSCc2cnc(C(C)C)c-3c2)[C@@H](C)C1]

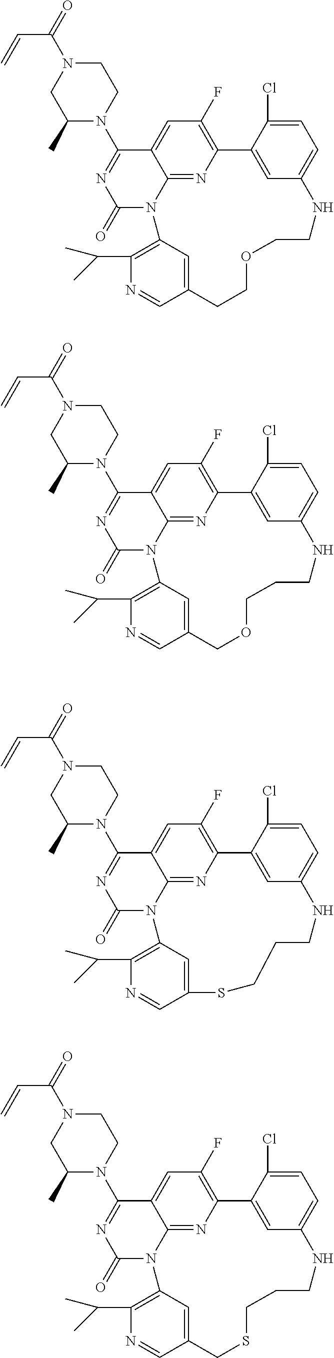 [SMILES: C=CC(=O)N1CCN(c2nc(=O)n3c4nc(c(F)cc24)-c2cc(ccc2Cl)NCCOCCc2cnc(C(C)C)c-3c2)[C@@H](C)C1 ; C=CC(=O)N1CCN(c2nc(=O)n3c4nc(c(F)cc24)-c2cc(ccc2Cl)NCCCOCc2cnc(C(C)C)c-3c2)[C@@H](C)C1 ; C=CC(=O)N1CCN(c2nc(=O)n3c4nc(c(F)cc24)-c2cc(ccc2Cl)NCCCSc2cnc(C(C)C)c-3c2)[C@@H](C)C1 ; C=CC(=O)N1CCN(c2nc(=O)n3c4nc(c(F)cc24)-c2cc(ccc2Cl)NCCCSCc2cnc(C(C)C)c-3c2)[C@@H](C)C1]

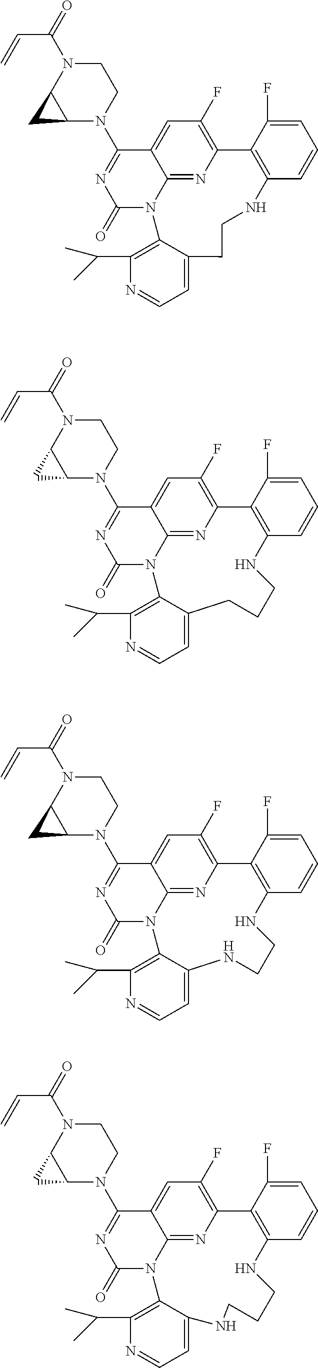 [SMILES: C=CC(=O)N1CCN(c2nc(=O)n3c4nc(c(F)cc24)-c2c(F)cccc2NCCc2ccnc(C(C)C)c2-3)[C@H]2C[C@H]21 ; C=CC(=O)N1CCN(c2nc(=O)n3c4nc(c(F)cc24)-c2c(F)cccc2NCCCc2ccnc(C(C)C)c2-3)[C@@H]2C[C@@H]21 ; C=CC(=O)N1CCN(c2nc(=O)n3c4nc(c(F)cc24)-c2c(F)cccc2NCCNc2ccnc(C(C)C)c2-3)[C@H]2C[C@H]21 ; C=CC(=O)N1CCN(c2nc(=O)n3c4nc(c(F)cc24)-c2c(F)cccc2NCCCNc2ccnc(C(C)C)c2-3)[C@@H]2C[C@@H]21]

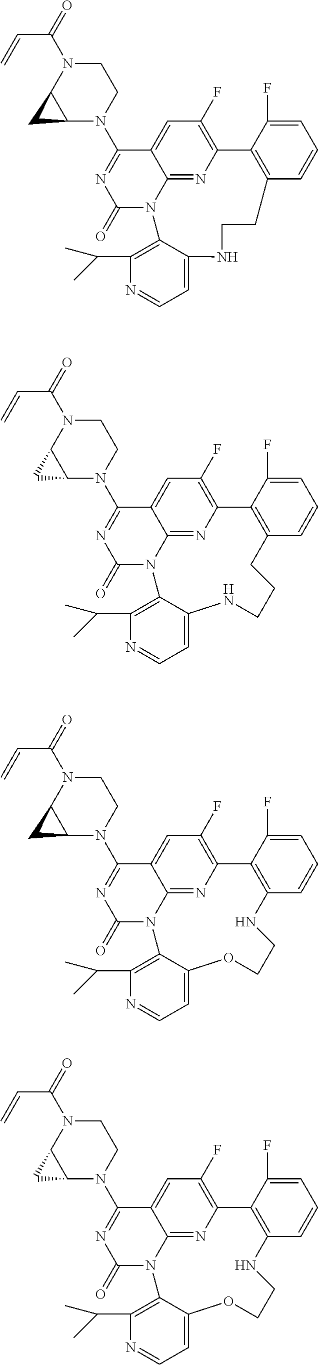 [SMILES: C=CC(=O)N1CCN(c2nc(=O)n3c4nc(c(F)cc24)-c2c(F)cccc2CCNc2ccnc(C(C)C)c2-3)[C@H]2C[C@H]21 ; C=CC(=O)N1CCN(c2nc(=O)n3c4nc(c(F)cc24)-c2c(F)cccc2CCCNc2ccnc(C(C)C)c2-3)[C@@H]2C[C@@H]21 ; C=CC(=O)N1CCN(c2nc(=O)n3c4nc(c(F)cc24)-c2c(F)cccc2NCCOc2ccnc(C(C)C)c2-3)[C@H]2C[C@H]21 ; C=CC(=O)N1CCN(c2nc(=O)n3c4nc(c(F)cc24)-c2c(F)cccc2NCCOc2ccnc(C(C)C)c2-3)[C@@H]2C[C@@H]21]

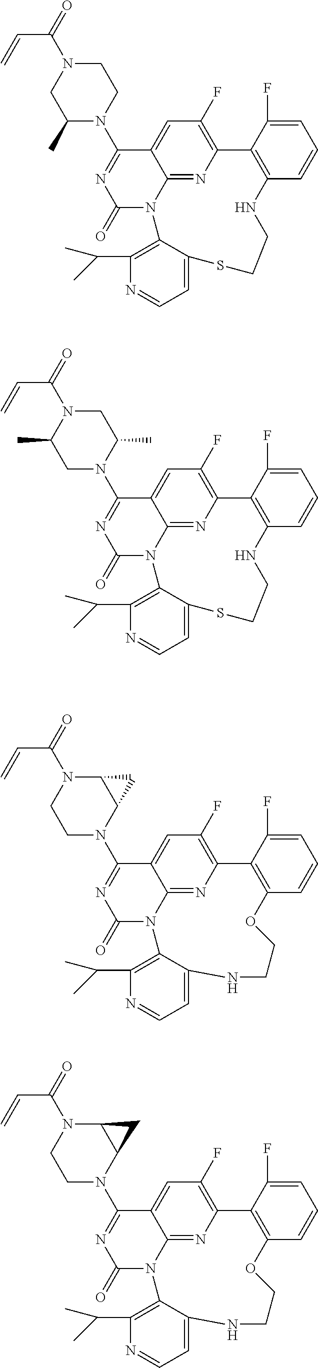 [SMILES: C=CC(=O)N1CCN(c2nc(=O)n3c4nc(c(F)cc24)-c2c(F)cccc2NCCSc2ccnc(C(C)C)c2-3)[C@@H](C)C1 ; C=CC(=O)N1C[C@H](C)N(c2nc(=O)n3c4nc(c(F)cc24)-c2c(F)cccc2NCCSc2ccnc(C(C)C)c2-3)C[C@H]1C ; C=CC(=O)N1CCN(c2nc(=O)n3c4nc(c(F)cc24)-c2c(F)cccc2OCCNc2ccnc(C(C)C)c2-3)[C@H]2C[C@H]21 ; C=CC(=O)N1CCN(c2nc(=O)n3c4nc(c(F)cc24)-c2c(F)cccc2OCCNc2ccnc(C(C)C)c2-3)[C@@H]2C[C@@H]21]

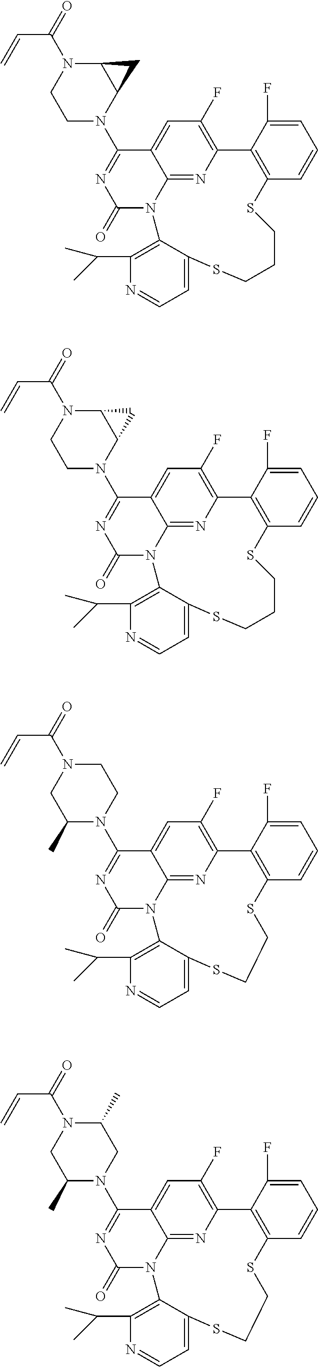 [SMILES: C=CC(=O)N1CCN(c2nc(=O)n3c4nc(c(F)cc24)-c2c(F)cccc2SCCCSc2ccnc(C(C)C)c2-3)[C@@H]2C[C@@H]21 ; C=CC(=O)N1CCN(c2nc(=O)n3c4nc(c(F)cc24)-c2c(F)cccc2SCCCSc2ccnc(C(C)C)c2-3)[C@H]2C[C@H]21 ; C=CC(=O)N1CCN(c2nc(=O)n3c4nc(c(F)cc24)-c2c(F)cccc2SCCSc2ccnc(C(C)C)c2-3)[C@@H](C)C1 ; C=CC(=O)N1C[C@H](C)N(c2nc(=O)n3c4nc(c(F)cc24)-c2c(F)cccc2SCCSc2ccnc(C(C)C)c2-3)C[C@H]1C]

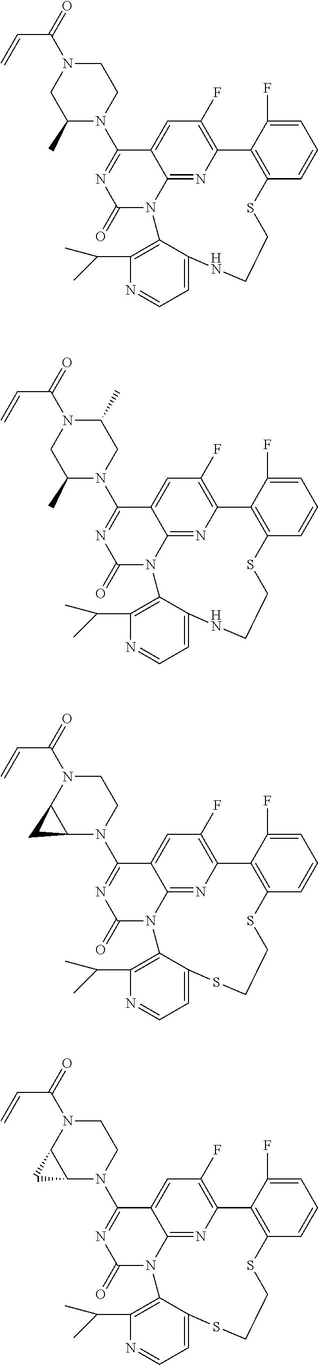 [SMILES: C=CC(=O)N1CCN(c2nc(=O)n3c4nc(c(F)cc24)-c2c(F)cccc2SCCNc2ccnc(C(C)C)c2-3)[C@@H](C)C1 ; C=CC(=O)N1C[C@H](C)N(c2nc(=O)n3c4nc(c(F)cc24)-c2c(F)cccc2SCCNc2ccnc(C(C)C)c2-3)C[C@H]1C ; C=CC(=O)N1CCN(c2nc(=O)n3c4nc(c(F)cc24)-c2c(F)cccc2SCCSc2ccnc(C(C)C)c2-3)[C@H]2C[C@H]21 ; C=CC(=O)N1CCN(c2nc(=O)n3c4nc(c(F)cc24)-c2c(F)cccc2SCCSc2ccnc(C(C)C)c2-3)[C@@H]2C[C@@H]21]

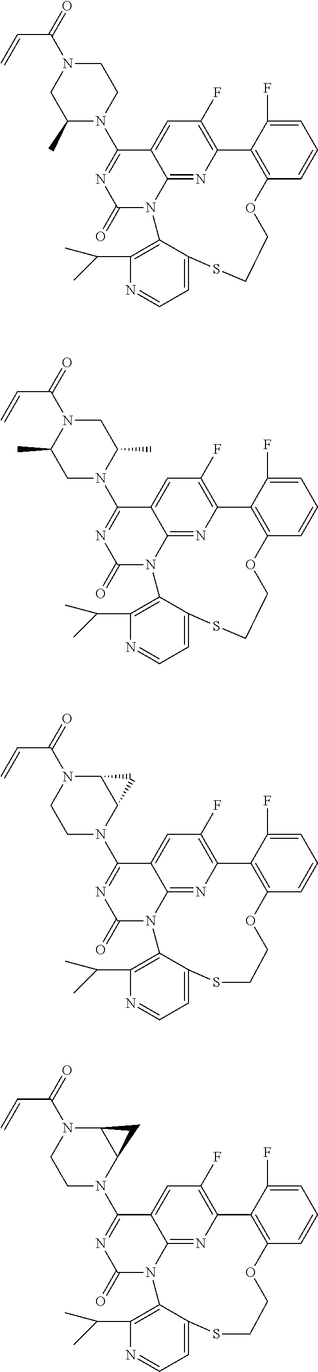 [SMILES: C=CC(=O)N1CCN(c2nc(=O)n3c4nc(c(F)cc24)-c2c(F)cccc2OCCSc2ccnc(C(C)C)c2-3)[C@@H](C)C1 ; C=CC(=O)N1C[C@H](C)N(c2nc(=O)n3c4nc(c(F)cc24)-c2c(F)cccc2OCCSc2ccnc(C(C)C)c2-3)C[C@H]1C ; C=CC(=O)N1CCN(c2nc(=O)n3c4nc(c(F)cc24)-c2c(F)cccc2OCCSc2ccnc(C(C)C)c2-3)[C@H]2C[C@H]21 ; C=CC(=O)N1CCN(c2nc(=O)n3c4nc(c(F)cc24)-c2c(F)cccc2OCCSc2ccnc(C(C)C)c2-3)[C@@H]2C[C@@H]21]

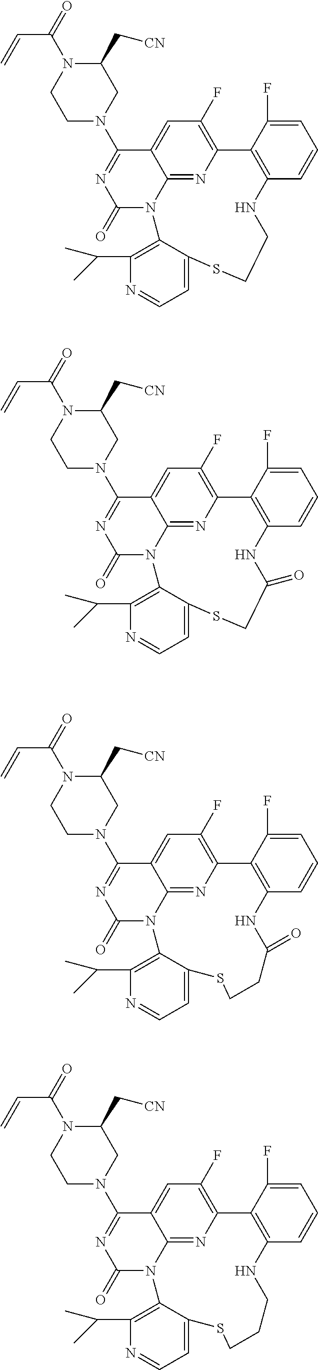 [SMILES: C=CC(=O)N1CCN(c2nc(=O)n3c4nc(c(F)cc24)-c2c(F)cccc2NCCSc2ccnc(C(C)C)c2-3)C[C@@H]1CC#N ; C=CC(=O)N1CCN(c2nc(=O)n3c4nc(c(F)cc24)-c2c(F)cccc2NC(=O)CSc2ccnc(C(C)C)c2-3)C[C@@H]1CC#N ; C=CC(=O)N1CCN(c2nc(=O)n3c4nc(c(F)cc24)-c2c(F)cccc2NC(=O)CCSc2ccnc(C(C)C)c2-3)C[C@@H]1CC#N ; C=CC(=O)N1CCN(c2nc(=O)n3c4nc(c(F)cc24)-c2c(F)cccc2NCCCSc2ccnc(C(C)C)c2-3)C[C@@H]1CC#N]

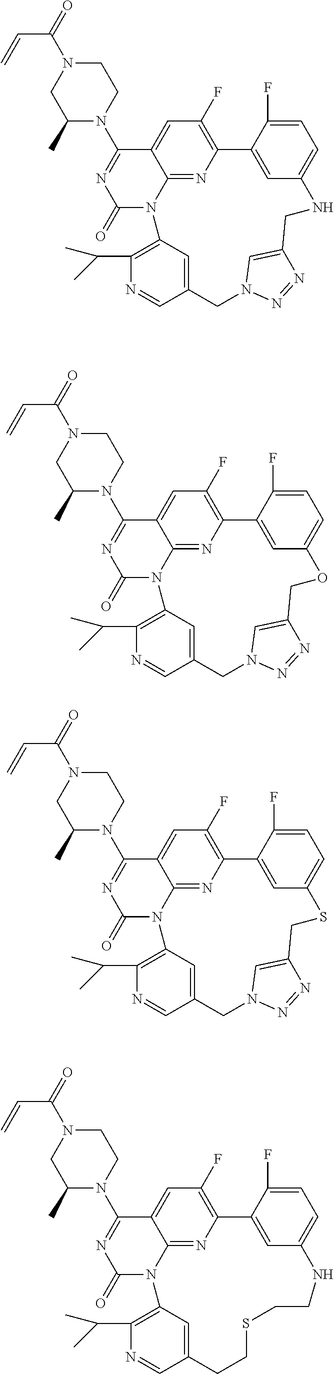 [SMILES: C=CC(=O)N1CCN(c2nc(=O)n3c4nc(c(F)cc24)-c2cc(ccc2F)NCc2cn(nn2)Cc2cnc(C(C)C)c-3c2)[C@@H](C)C1 ; C=CC(=O)N1CCN(c2nc(=O)n3c4nc(c(F)cc24)-c2cc(ccc2F)OCc2cn(nn2)Cc2cnc(C(C)C)c-3c2)[C@@H](C)C1 ; C=CC(=O)N1CCN(c2nc(=O)n3c4nc(c(F)cc24)-c2cc(ccc2F)SCc2cn(nn2)Cc2cnc(C(C)C)c-3c2)[C@@H](C)C1 ; C=CC(=O)N1CCN(c2nc(=O)n3c4nc(c(F)cc24)-c2cc(ccc2F)NCCSCCc2cnc(C(C)C)c-3c2)[C@@H](C)C1]

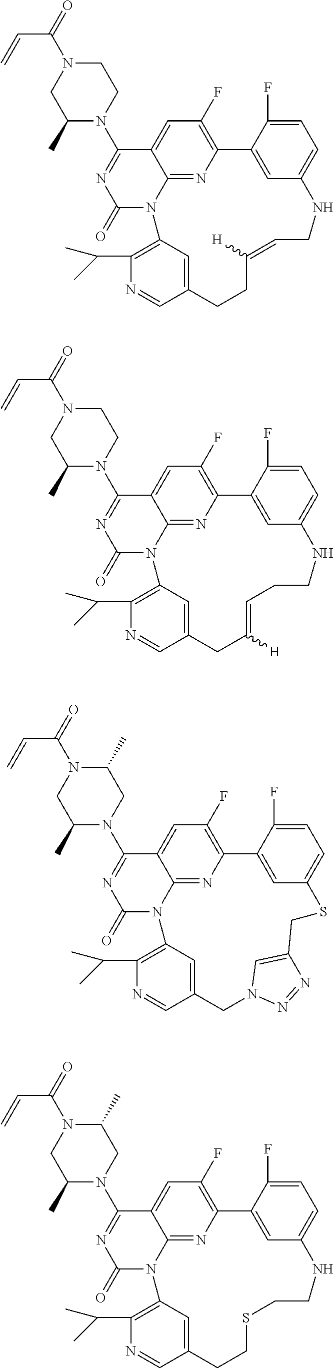 [SMILES: C=CC(=O)N1CCN(c2nc(=O)n3c4nc(c(F)cc24)-c2cc(ccc2F)NCC=CCCc2cnc(C(C)C)c-3c2)[C@@H](C)C1 ; C=CC(=O)N1CCN(c2nc(=O)n3c4nc(c(F)cc24)-c2cc(ccc2F)NCCC=CCc2cnc(C(C)C)c-3c2)[C@@H](C)C1 ; C=CC(=O)N1C[C@H](C)N(c2nc(=O)n3c4nc(c(F)cc24)-c2cc(ccc2F)SCc2cn(nn2)Cc2cnc(C(C)C)c-3c2)C[C@H]1C ; C=CC(=O)N1C[C@H](C)N(c2nc(=O)n3c4nc(c(F)cc24)-c2cc(ccc2F)NCCSCCc2cnc(C(C)C)c-3c2)C[C@H]1C]

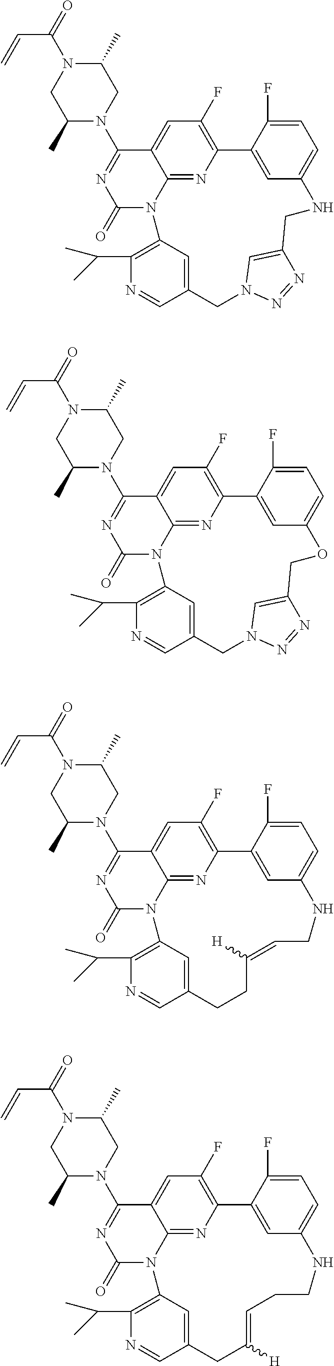 [SMILES: C=CC(=O)N1C[C@H](C)N(c2nc(=O)n3c4nc(c(F)cc24)-c2cc(ccc2F)NCc2cn(nn2)Cc2cnc(C(C)C)c-3c2)C[C@H]1C ; C=CC(=O)N1C[C@H](C)N(c2nc(=O)n3c4nc(c(F)cc24)-c2cc(ccc2F)OCc2cn(nn2)Cc2cnc(C(C)C)c-3c2)C[C@H]1C ; C=CC(=O)N1C[C@H](C)N(c2nc(=O)n3c4nc(c(F)cc24)-c2cc(ccc2F)NCC=CCCc2cnc(C(C)C)c-3c2)C[C@H]1C ; C=CC(=O)N1C[C@H](C)N(c2nc(=O)n3c4nc(c(F)cc24)-c2cc(ccc2F)NCCC=CCc2cnc(C(C)C)c-3c2)C[C@H]1C]

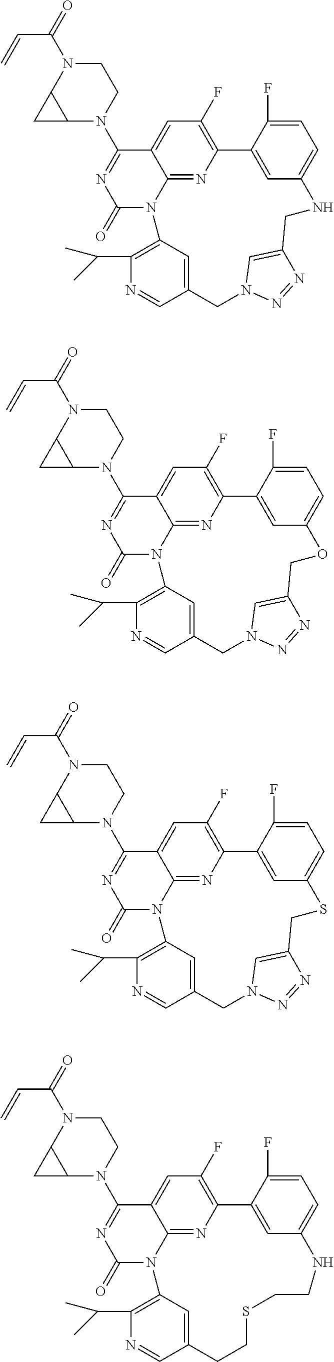 [SMILES: C=CC(=O)N1CCN(c2nc(=O)n3c4nc(c(F)cc24)-c2cc(ccc2F)NCc2cn(nn2)Cc2cnc(C(C)C)c-3c2)C2CC21 ; C=CC(=O)N1CCN(c2nc(=O)n3c4nc(c(F)cc24)-c2cc(ccc2F)OCc2cn(nn2)Cc2cnc(C(C)C)c-3c2)C2CC21 ; C=CC(=O)N1CCN(c2nc(=O)n3c4nc(c(F)cc24)-c2cc(ccc2F)SCc2cn(nn2)Cc2cnc(C(C)C)c-3c2)C2CC21 ; C=CC(=O)N1CCN(c2nc(=O)n3c4nc(c(F)cc24)-c2cc(ccc2F)NCCSCCc2cnc(C(C)C)c-3c2)C2CC21]

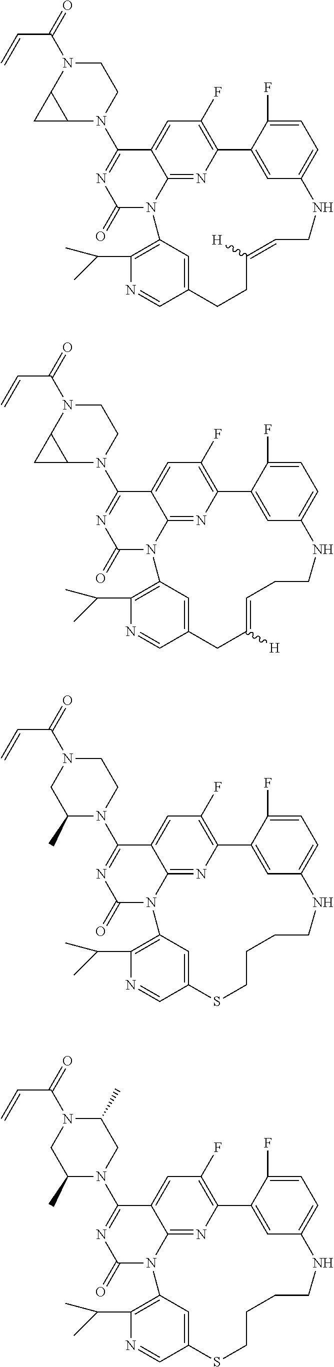 [SMILES: C=CC(=O)N1CCN(c2nc(=O)n3c4nc(c(F)cc24)-c2cc(ccc2F)NCC=CCCc2cnc(C(C)C)c-3c2)C2CC21 ; C=CC(=O)N1CCN(c2nc(=O)n3c4nc(c(F)cc24)-c2cc(ccc2F)NCCC=CCc2cnc(C(C)C)c-3c2)C2CC21 ; C=CC(=O)N1CCN(c2nc(=O)n3c4nc(c(F)cc24)-c2cc(ccc2F)NCCCCSc2cnc(C(C)C)c-3c2)[C@@H](C)C1 ; C=CC(=O)N1C[C@H](C)N(c2nc(=O)n3c4nc(c(F)cc24)-c2cc(ccc2F)NCCCCSc2cnc(C(C)C)c-3c2)C[C@H]1C]

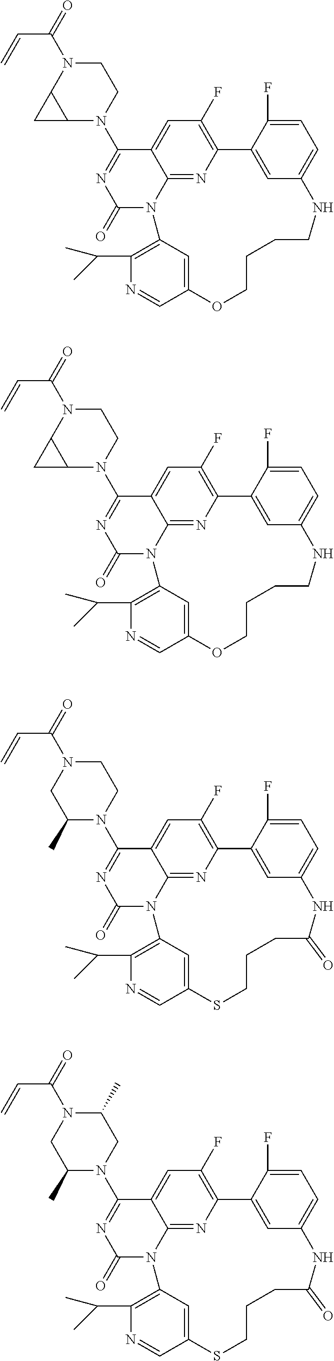 [SMILES: C=CC(=O)N1CCN(c2nc(=O)n3c4nc(c(F)cc24)-c2cc(ccc2F)NCCCCOc2cnc(C(C)C)c-3c2)C2CC21 ; C=CC(=O)N1CCN(c2nc(=O)n3c4nc(c(F)cc24)-c2cc(ccc2F)NCCCCOc2cnc(C(C)C)c-3c2)C2CC21 ; C=CC(=O)N1CCN(c2nc(=O)n3c4nc(c(F)cc24)-c2cc(ccc2F)NC(=O)CCCSc2cnc(C(C)C)c-3c2)[C@@H](C)C1 ; C=CC(=O)N1C[C@H](C)N(c2nc(=O)n3c4nc(c(F)cc24)-c2cc(ccc2F)NC(=O)CCCSc2cnc(C(C)C)c-3c2)C[C@H]1C]

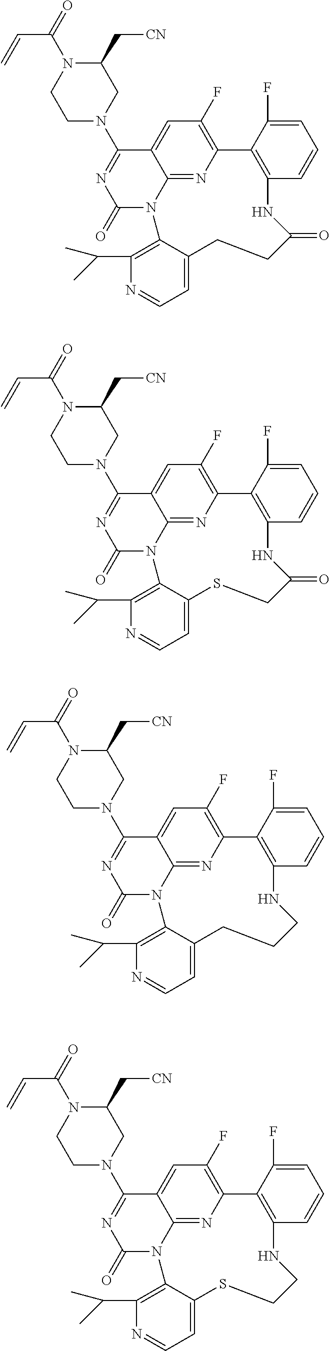 [SMILES: C=CC(=O)N1CCN(c2nc(=O)n3c4nc(c(F)cc24)-c2c(F)cccc2NC(=O)CCc2ccnc(C(C)C)c2-3)C[C@@H]1CC#N ; C=CC(=O)N1CCN(c2nc(=O)n3c4nc(c(F)cc24)-c2c(F)cccc2NC(=O)CSc2ccnc(C(C)C)c2-3)C[C@@H]1CC#N ; C=CC(=O)N1CCN(c2nc(=O)n3c4nc(c(F)cc24)-c2c(F)cccc2NCCCc2ccnc(C(C)C)c2-3)C[C@@H]1CC#N ; C=CC(=O)N1CCN(c2nc(=O)n3c4nc(c(F)cc24)-c2c(F)cccc2NCCSc2ccnc(C(C)C)c2-3)C[C@@H]1CC#N]

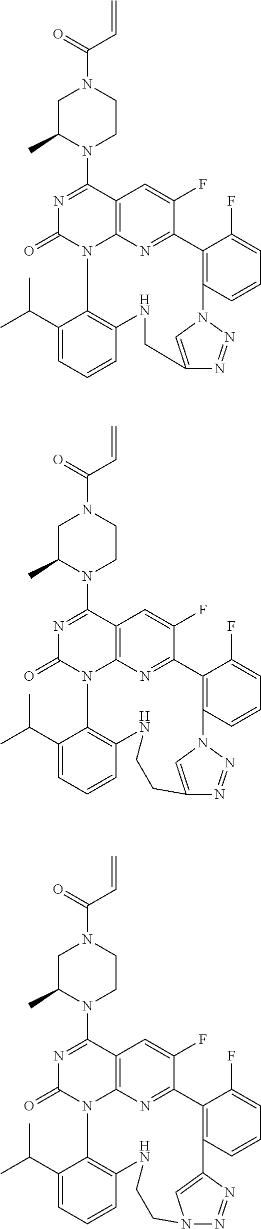 [SMILES: C=CC(=O)N1CCN(c2nc(=O)n3c4nc(c(F)cc24)-c2c(F)cccc2-n2cc(nn2)CNc2cccc(C(C)C)c2-3)[C@@H](C)C1 ; C=CC(=O)N1CCN(c2nc(=O)n3c4nc(c(F)cc24)-c2c(F)cccc2-n2cc(nn2)CCNc2cccc(C(C)C)c2-3)[C@@H](C)C1 ; C=CC(=O)N1CCN(c2nc(=O)n3c4nc(c(F)cc24)-c2c(F)cccc2-c2cn(nn2)CCNc2cccc(C(C)C)c2-3)[C@@H](C)C1]

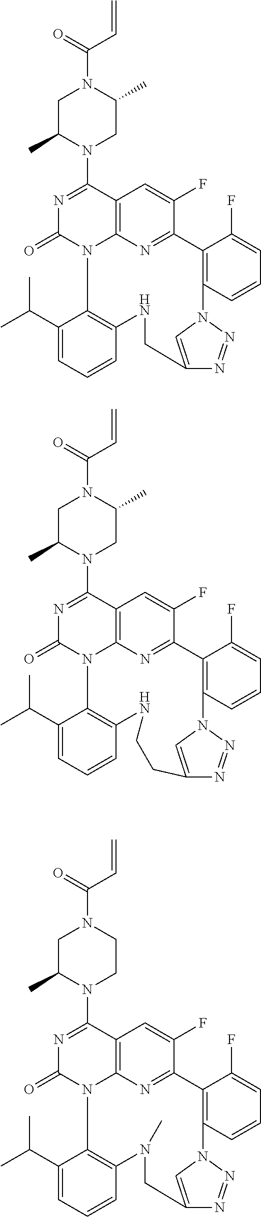

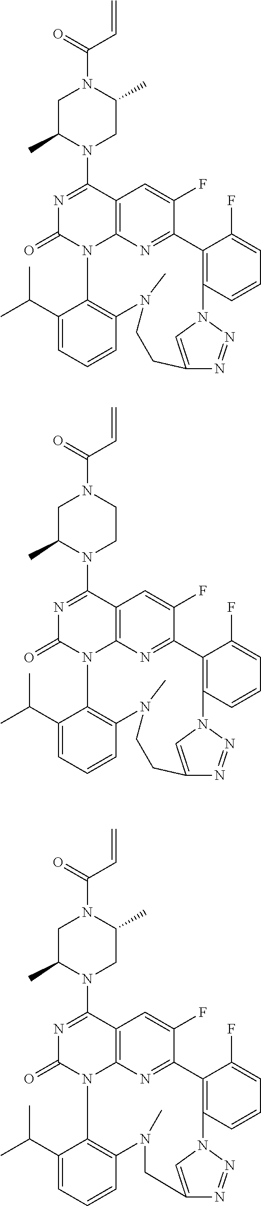 [SMILES: C=CC(=O)N1C[C@H](C)N(c2nc(=O)n3c4nc(c(F)cc24)-c2c(F)cccc2-n2cc(nn2)CCN(C)c2cccc(C(C)C)c2-3)C[C@H]1C ; C=CC(=O)N1CCN(c2nc(=O)n3c4nc(c(F)cc24)-c2c(F)cccc2-n2cc(nn2)CCN(C)c2cccc(C(C)C)c2-3)[C@@H](C)C1 ; C=CC(=O)N1C[C@H](C)N(c2nc(=O)n3c4nc(c(F)cc24)-c2c(F)cccc2-n2cc(nn2)CN(C)c2cccc(C(C)C)c2-3)C[C@H]1C]

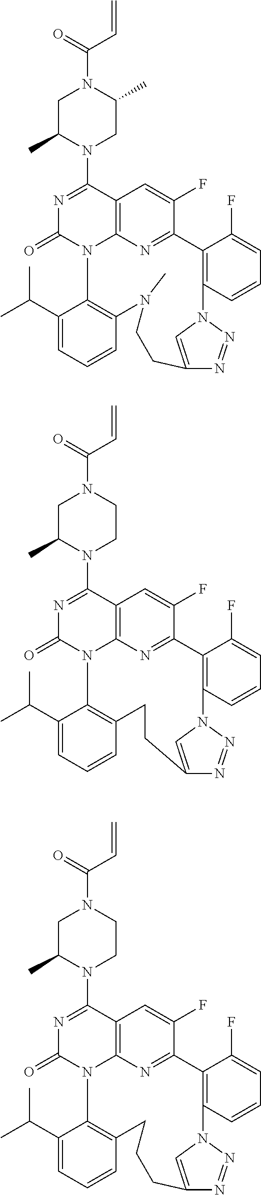 [SMILES: C=CC(=O)N1C[C@H](C)N(c2nc(=O)n3c4nc(c(F)cc24)-c2c(F)cccc2-n2cc(nn2)CCN(C)c2cccc(C(C)C)c2-3)C[C@H]1C ; C=CC(=O)N1CCN(c2nc(=O)n3c4nc(c(F)cc24)-c2c(F)cccc2-n2cc(nn2)CCc2cccc(C(C)C)c2-3)[C@@H](C)C1 ; C=CC(=O)N1CCN(c2nc(=O)n3c4nc(c(F)cc24)-c2c(F)cccc2-n2cc(nn2)CCCc2cccc(C(C)C)c2-3)[C@@H](C)C1]

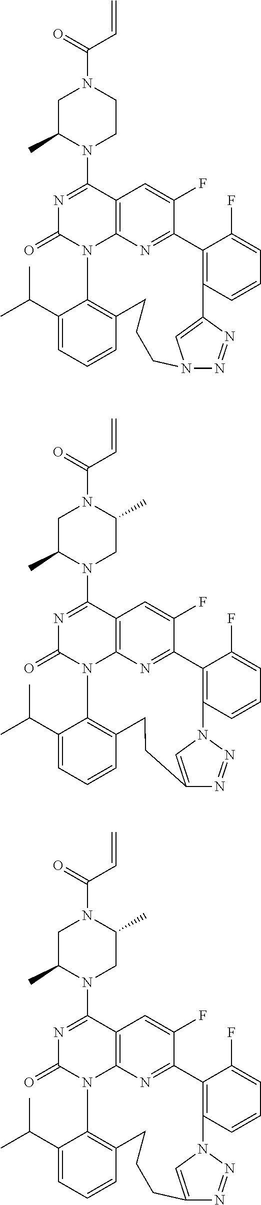 [SMILES: C=CC(=O)N1CCN(c2nc(=O)n3c4nc(c(F)cc24)-c2c(F)cccc2-c2cn(nn2)CCCc2cccc(C(C)C)c2-3)[C@@H](C)C1 ; C=CC(=O)N1C[C@H](C)N(c2nc(=O)n3c4nc(c(F)cc24)-c2c(F)cccc2-n2cc(nn2)CCc2cccc(C(C)C)c2-3)C[C@H]1C ; C=CC(=O)N1C[C@H](C)N(c2nc(=O)n3c4nc(c(F)cc24)-c2c(F)cccc2-n2cc(nn2)CCCc2cccc(C(C)C)c2-3)C[C@H]1C]

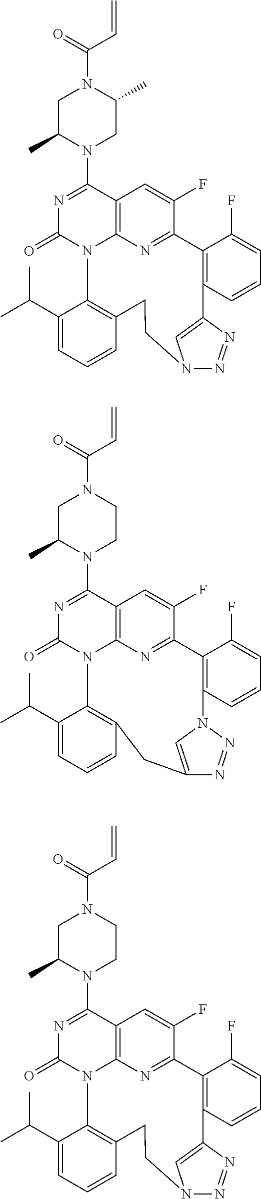 [SMILES: C=CC(=O)N1C[C@H](C)N(c2nc(=O)n3c4nc(c(F)cc24)-c2c(F)cccc2-c2cn(nn2)CCc2cccc(C(C)C)c2-3)C[C@H]1C ; C=CC(=O)N1CCN(c2nc(=O)n3c4nc(c(F)cc24)-c2c(F)cccc2-n2cc(nn2)Cc2cccc(C(C)C)c2-3)[C@@H](C)C1 ; C=CC(=O)N1CCN(c2nc(=O)n3c4nc(c(F)cc24)-c2c(F)cccc2-c2cn(nn2)CCc2cccc(C(C)C)c2-3)[C@@H](C)C1]

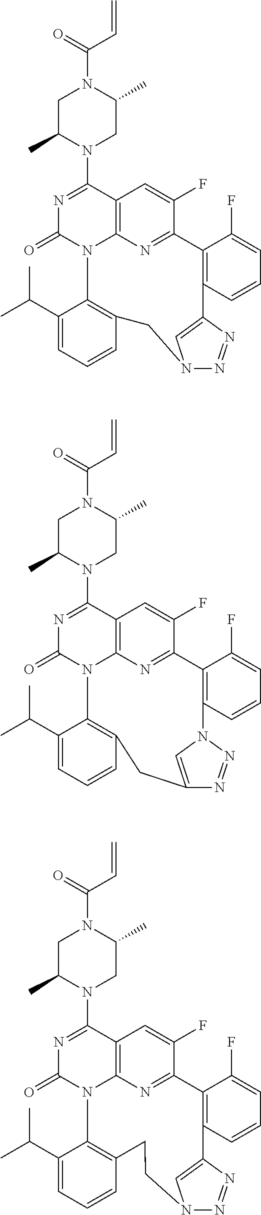 [SMILES: C=CC(=O)N1C[C@H](C)N(c2nc(=O)n3c4nc(c(F)cc24)-c2c(F)cccc2-c2cn(nn2)Cc2cccc(C(C)C)c2-3)C[C@H]1C ; C=CC(=O)N1C[C@H](C)N(c2nc(=O)n3c4nc(c(F)cc24)-c2c(F)cccc2-n2cc(nn2)Cc2cccc(C(C)C)c2-3)C[C@H]1C ; C=CC(=O)N1C[C@H](C)N(c2nc(=O)n3c4nc(c(F)cc24)-c2c(F)cccc2-c2cn(nn2)CCc2cccc(C(C)C)c2-3)C[C@H]1C]

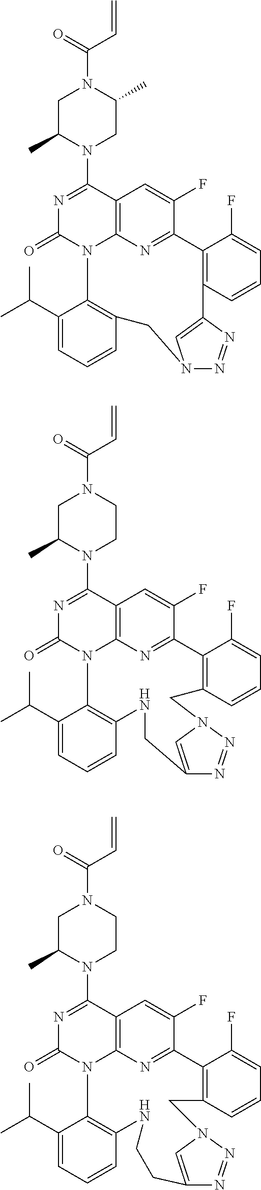 [SMILES: C=CC(=O)N1C[C@H](C)N(c2nc(=O)n3c4nc(c(F)cc24)-c2c(F)cccc2-c2cn(nn2)Cc2cccc(C(C)C)c2-3)C[C@H]1C ; C=CC(=O)N1CCN(c2nc(=O)n3c4nc(c(F)cc24)-c2c(F)cccc2Cn2cc(nn2)CNc2cccc(C(C)C)c2-3)[C@@H](C)C1 ; C=CC(=O)N1CCN(c2nc(=O)n3c4nc(c(F)cc24)-c2c(F)cccc2Cn2cc(nn2)CCNc2cccc(C(C)C)c2-3)[C@@H](C)C1]

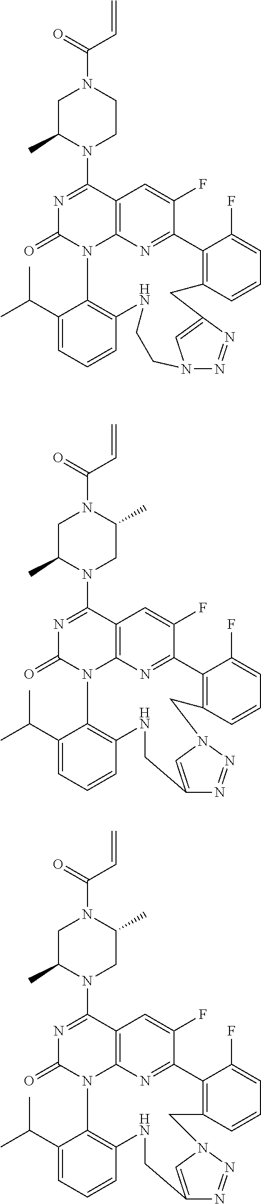 [SMILES: C=CC(=O)N1CCN(c2nc(=O)n3c4nc(c(F)cc24)-c2c(F)cccc2Cc2cn(nn2)CCNc2cccc(C(C)C)c2-3)[C@@H](C)C1 ; C=CC(=O)N1C[C@H](C)N(c2nc(=O)n3c4nc(c(F)cc24)-c2c(F)cccc2Cn2cc(nn2)CNc2cccc(C(C)C)c2-3)C[C@H]1C ; C=CC(=O)N1C[C@H](C)N(c2nc(=O)n3c4nc(c(F)cc24)-c2c(F)cccc2Cn2cc(nn2)CNc2cccc(C(C)C)c2-3)C[C@H]1C]

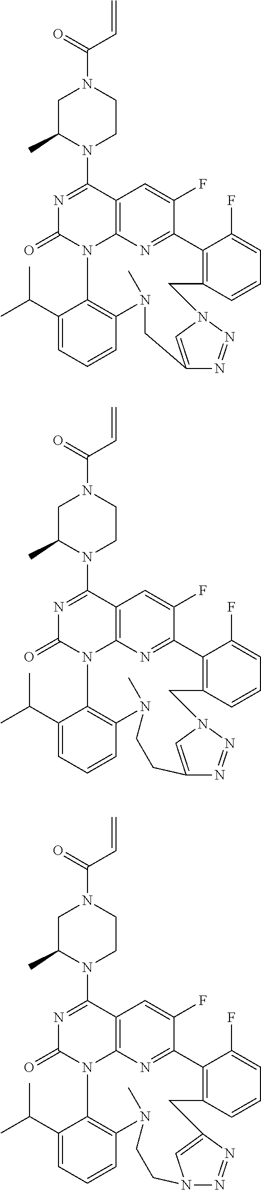 [SMILES: C=CC(=O)N1CCN(c2nc(=O)n3c4nc(c(F)cc24)-c2c(F)cccc2Cn2cc(nn2)CN(C)c2cccc(C(C)C)c2-3)[C@@H](C)C1 ; C=CC(=O)N1CCN(c2nc(=O)n3c4nc(c(F)cc24)-c2c(F)cccc2Cn2cc(nn2)CCN(C)c2cccc(C(C)C)c2-3)[C@@H](C)C1 ; C=CC(=O)N1CCN(c2nc(=O)n3c4nc(c(F)cc24)-c2c(F)cccc2Cc2cn(nn2)CCN(C)c2cccc(C(C)C)c2-3)[C@@H](C)C1]

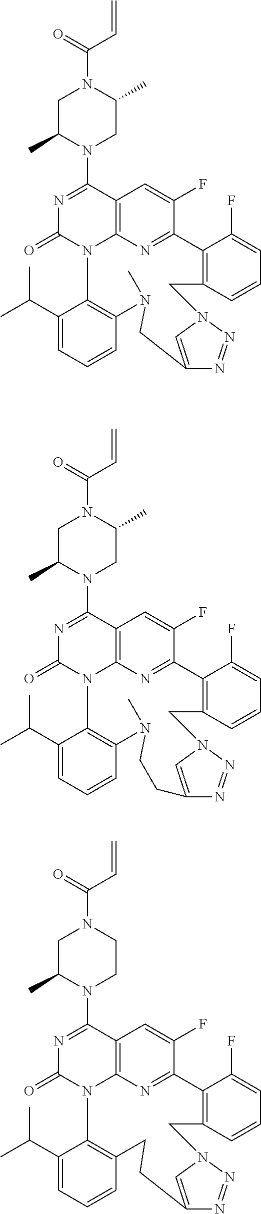 [SMILES: C=CC(=O)N1C[C@H](C)N(c2nc(=O)n3c4nc(c(F)cc24)-c2c(F)cccc2Cn2cc(nn2)CN(C)c2cccc(C(C)C)c2-3)C[C@H]1C ; C=CC(=O)N1C[C@H](C)N(c2nc(=O)n3c4nc(c(F)cc24)-c2c(F)cccc2Cn2cc(nn2)CCN(C)c2cccc(C(C)C)c2-3)C[C@H]1C ; C=CC(=O)N1CCN(c2nc(=O)n3c4nc(c(F)cc24)-c2c(F)cccc2Cn2cc(nn2)CCc2cccc(C(C)C)c2-3)[C@@H](C)C1]

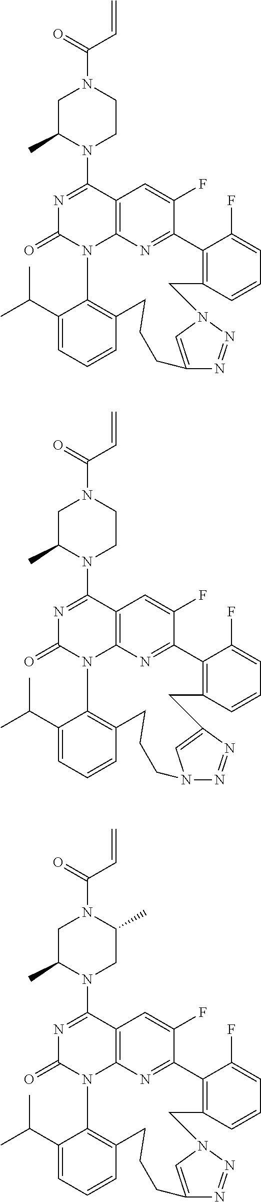 [SMILES: C=CC(=O)N1CCN(c2nc(=O)n3c4nc(c(F)cc24)-c2c(F)cccc2Cn2cc(nn2)CCCc2cccc(C(C)C)c2-3)[C@@H](C)C1 ; C=CC(=O)N1CCN(c2nc(=O)n3c4nc(c(F)cc24)-c2c(F)cccc2Cc2cn(nn2)CCCc2cccc(C(C)C)c2-3)[C@@H](C)C1 ; C=CC(=O)N1C[C@H](C)N(c2nc(=O)n3c4nc(c(F)cc24)-c2c(F)cccc2Cn2cc(nn2)CCCc2cccc(C(C)C)c2-3)C[C@H]1C]

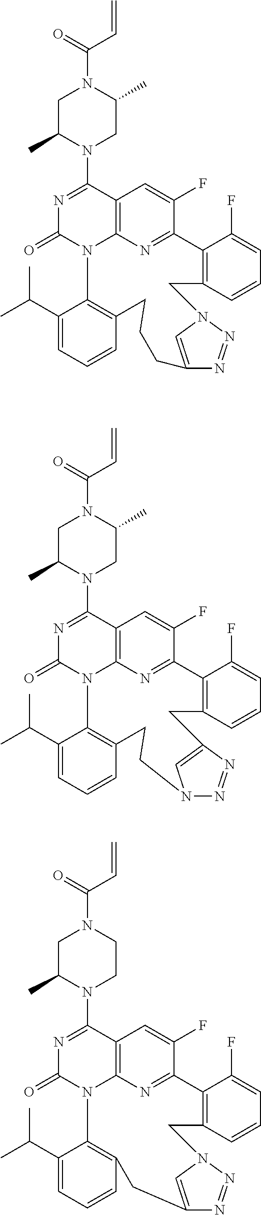 [SMILES: C=CC(=O)N1C[C@H](C)N(c2nc(=O)n3c4nc(c(F)cc24)-c2c(F)cccc2Cn2cc(nn2)CCCc2cccc(C(C)C)c2-3)C[C@H]1C ; C=CC(=O)N1C[C@H](C)N(c2nc(=O)n3c4nc(c(F)cc24)-c2c(F)cccc2Cc2cn(nn2)CCc2cccc(C(C)C)c2-3)C[C@H]1C ; C=CC(=O)N1CCN(c2nc(=O)n3c4nc(c(F)cc24)-c2c(F)cccc2Cn2cc(nn2)Cc2cccc(C(C)C)c2-3)[C@@H](C)C1]

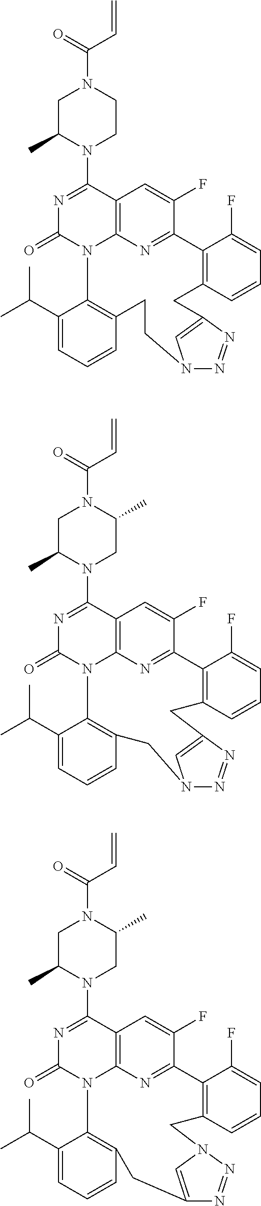 [SMILES: C=CC(=O)N1CCN(c2nc(=O)n3c4nc(c(F)cc24)-c2c(F)cccc2Cc2cn(nn2)CCc2cccc(C(C)C)c2-3)[C@@H](C)C1 ; C=CC(=O)N1C[C@H](C)N(c2nc(=O)n3c4nc(c(F)cc24)-c2c(F)cccc2Cc2cn(nn2)Cc2cccc(C(C)C)c2-3)C[C@H]1C ; C=CC(=O)N1C[C@H](C)N(c2nc(=O)n3c4nc(c(F)cc24)-c2c(F)cccc2Cn2cc(nn2)Cc2cccc(C(C)C)c2-3)C[C@H]1C]

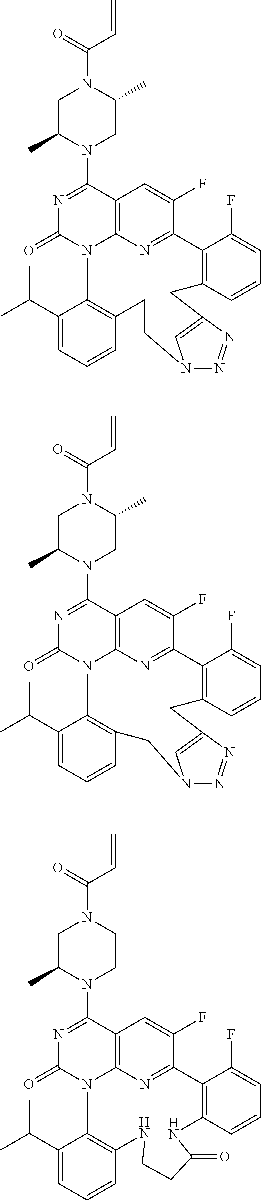 [SMILES: C=CC(=O)N1C[C@H](C)N(c2nc(=O)n3c4nc(c(F)cc24)-c2c(F)cccc2Cc2cn(nn2)CCc2cccc(C(C)C)c2-3)C[C@H]1C ; C=CC(=O)N1C[C@H](C)N(c2nc(=O)n3c4nc(c(F)cc24)-c2c(F)cccc2Cc2cn(nn2)Cc2cccc(C(C)C)c2-3)C[C@H]1C ; C=CC(=O)N1CCN(c2nc(=O)n3c4nc(c(F)cc24)-c2c(F)cccc2NC(=O)CCNc2cccc(C(C)C)c2-3)[C@@H](C)C1]

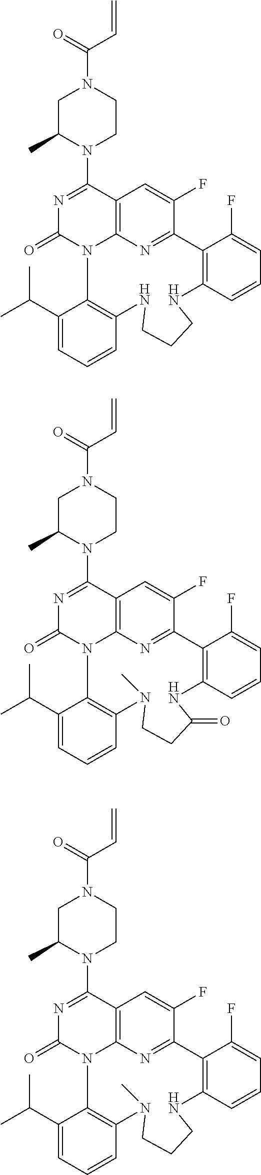

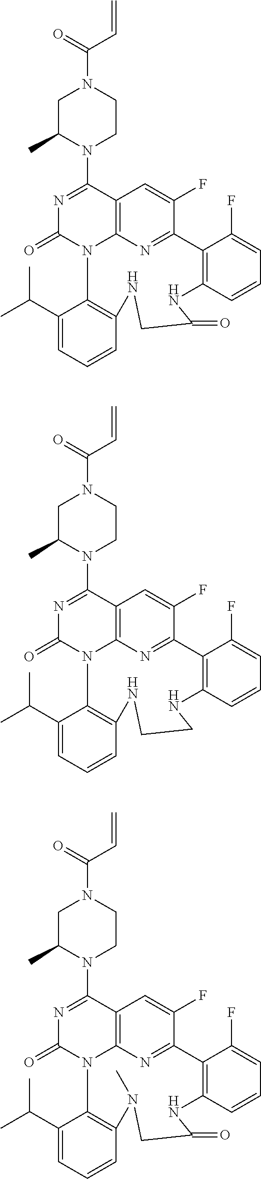 [SMILES: C=CC(=O)N1CCN(c2nc(=O)n3c4nc(c(F)cc24)-c2c(F)cccc2NC(=O)CNc2cccc(C(C)C)c2-3)[C@@H](C)C1 ; C=CC(=O)N1CCN(c2nc(=O)n3c4nc(c(F)cc24)-c2c(F)cccc2NCCNc2cccc(C(C)C)c2-3)[C@@H](C)C1 ; C=CC(=O)N1CCN(c2nc(=O)n3c4nc(c(F)cc24)-c2c(F)cccc2NC(=O)CN(C)c2cccc(C(C)C)c2-3)[C@@H](C)C1]

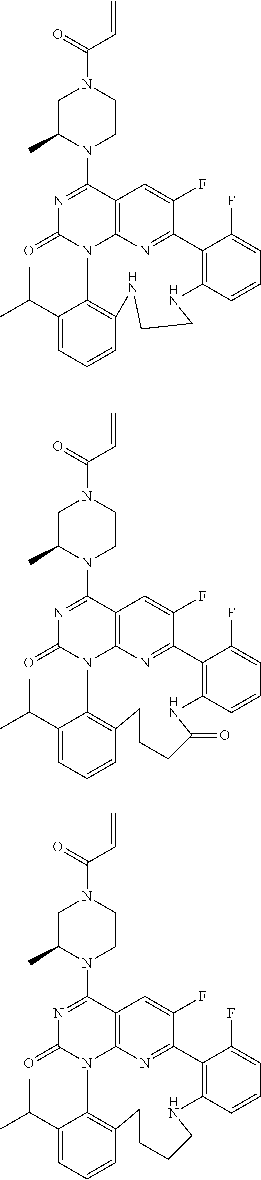

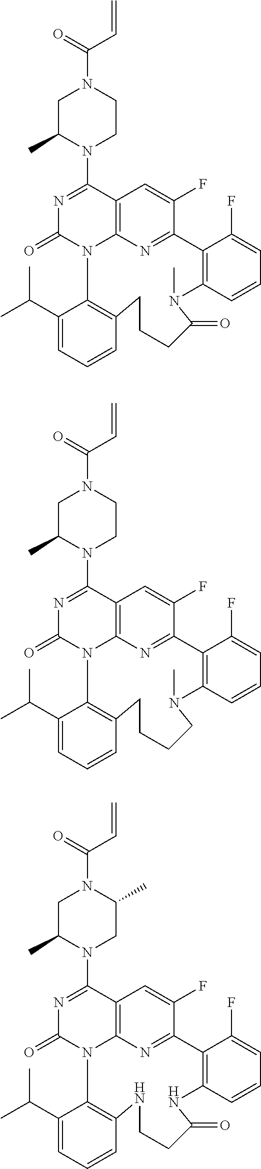

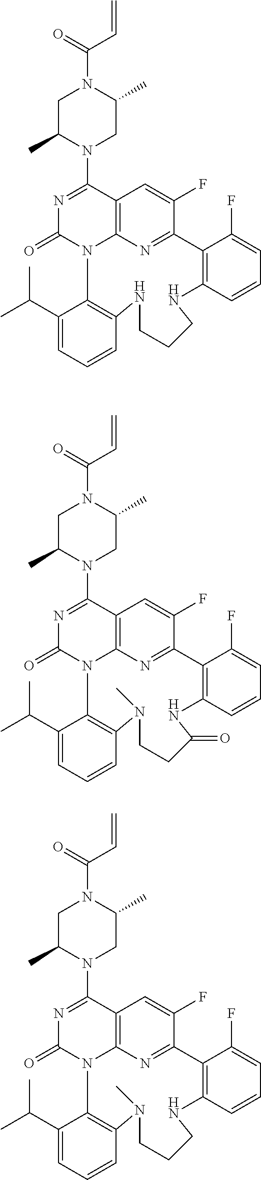

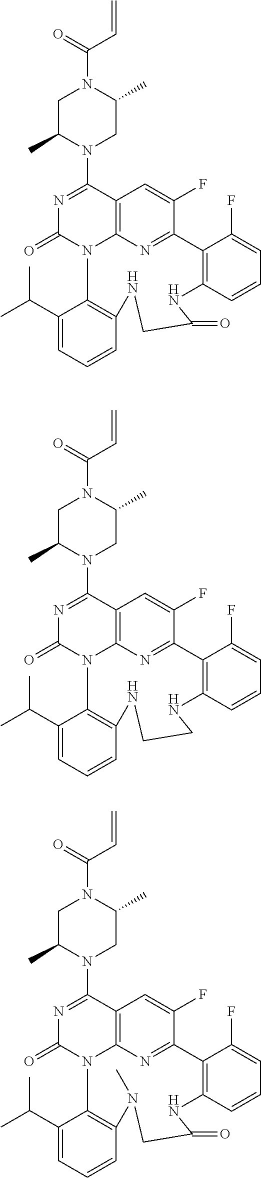

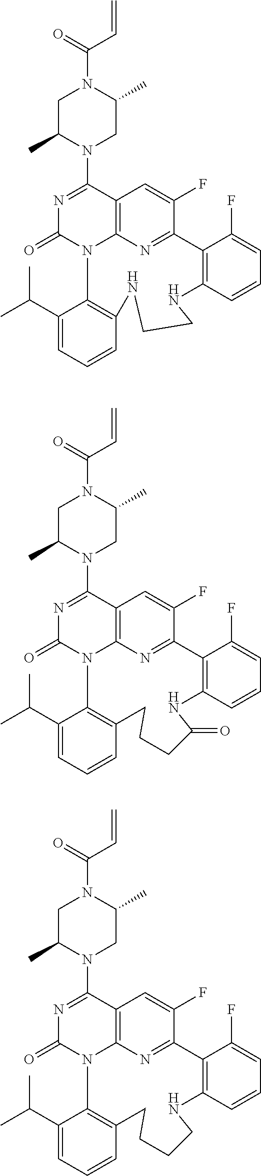

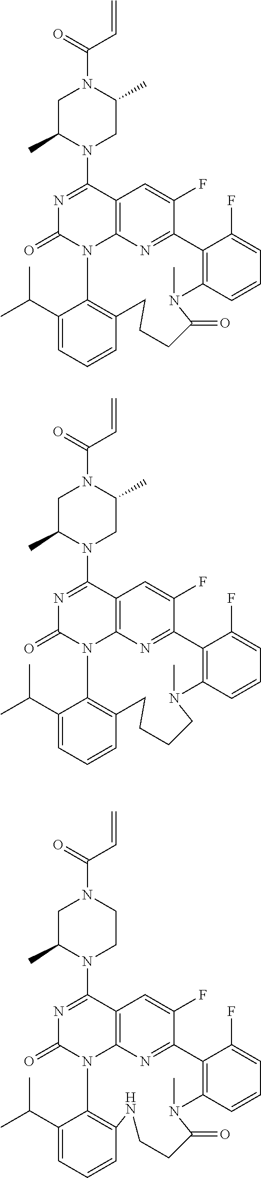

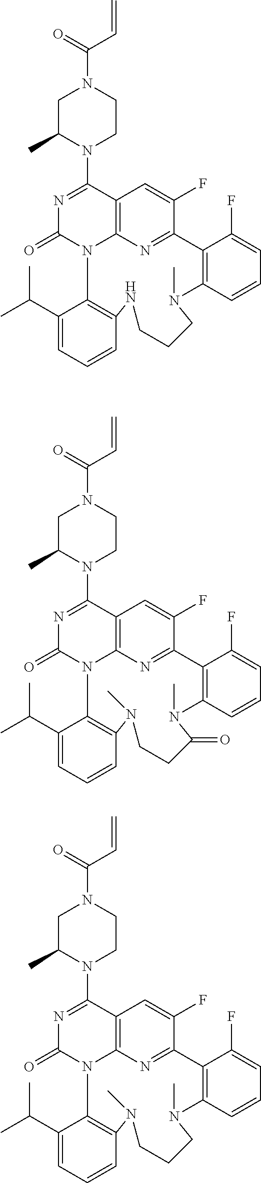

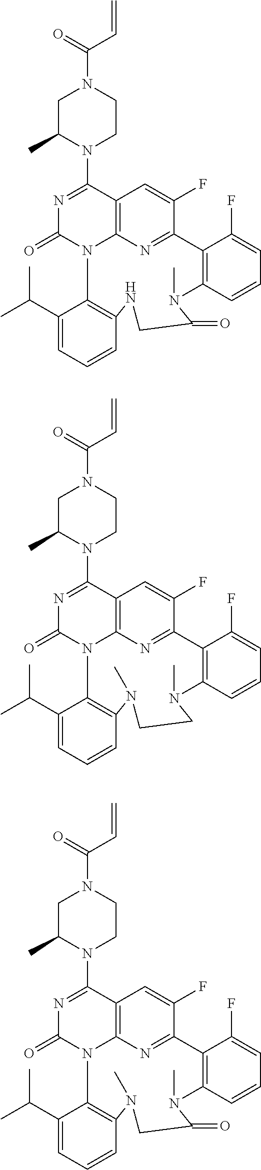

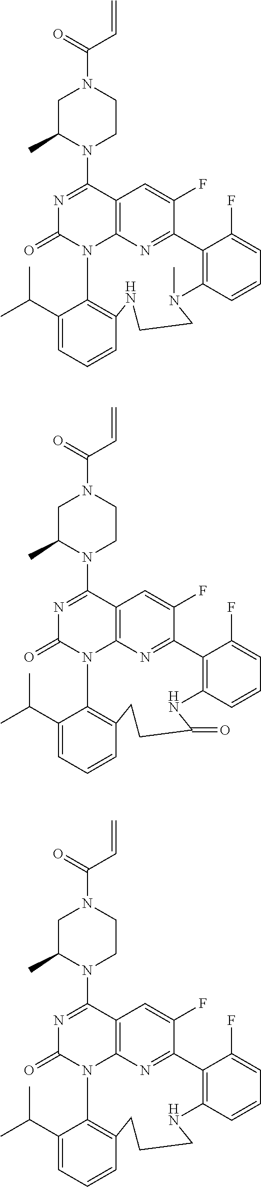 [SMILES: C=CC(=O)N1CCN(c2nc(=O)n3c4nc(c(F)cc24)-c2c(F)cccc2N(C)CCNc2cccc(C(C)C)c2-3)[C@@H](C)C1 ; C=CC(=O)N1CCN(c2nc(=O)n3c4nc(c(F)cc24)-c2c(F)cccc2NC(=O)CCc2cccc(C(C)C)c2-3)[C@@H](C)C1 ; C=CC(=O)N1CCN(c2nc(=O)n3c4nc(c(F)cc24)-c2c(F)cccc2NCCCc2cccc(C(C)C)c2-3)[C@@H](C)C1]

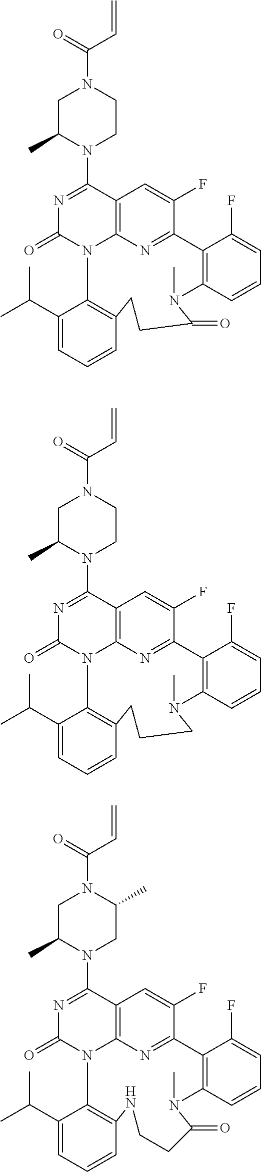

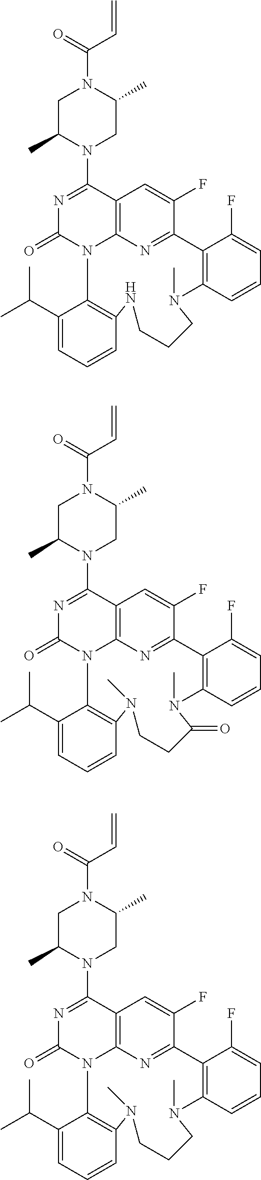 [SMILES: C=CC(=O)N1C[C@H](C)N(c2nc(=O)n3c4nc(c(F)cc24)-c2c(F)cccc2N(C)CCCNc2cccc(C(C)C)c2-3)C[C@H]1C ; C=CC(=O)N1C[C@H](C)N(c2nc(=O)n3c4nc(c(F)cc24)-c2c(F)cccc2N(C)C(=O)CCN(C)c2cccc(C(C)C)c2-3)C[C@H]1C ; C=CC(=O)N1C[C@H](C)N(c2nc(=O)n3c4nc(c(F)cc24)-c2c(F)cccc2N(C)CCCN(C)c2cccc(C(C)C)c2-3)C[C@H]1C]

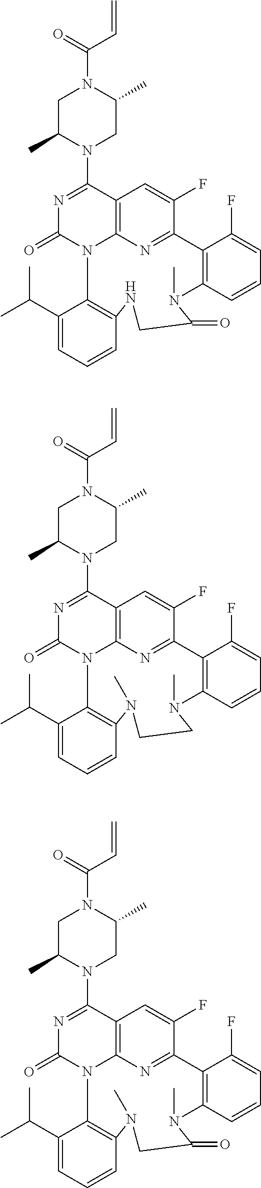 [SMILES: C=CC(=O)N1C[C@H](C)N(c2nc(=O)n3c4nc(c(F)cc24)-c2c(F)cccc2N(C)C(=O)CNc2cccc(C(C)C)c2-3)C[C@H]1C ; C=CC(=O)N1C[C@H](C)N(c2nc(=O)n3c4nc(c(F)cc24)-c2c(F)cccc2N(C)CCN(C)c2cccc(C(C)C)c2-3)C[C@H]1C ; C=CC(=O)N1C[C@H](C)N(c2nc(=O)n3c4nc(c(F)cc24)-c2c(F)cccc2N(C)C(=O)CN(C)c2cccc(C(C)C)c2-3)C[C@H]1C]

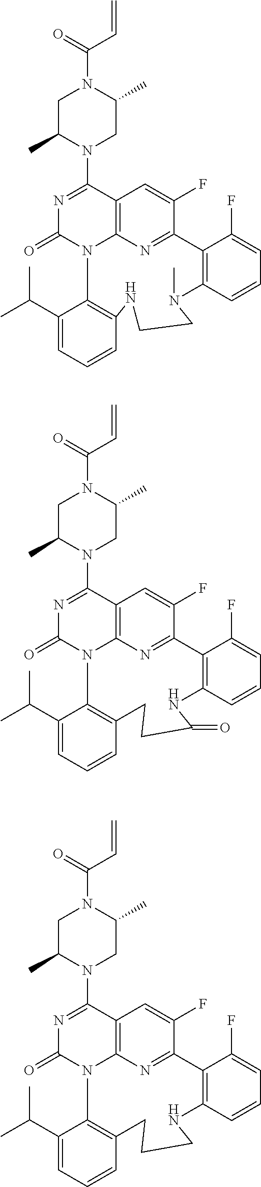 [SMILES: C=CC(=O)N1C[C@H](C)N(c2nc(=O)n3c4nc(c(F)cc24)-c2c(F)cccc2N(C)CCNc2cccc(C(C)C)c2-3)C[C@H]1C ; C=CC(=O)N1C[C@H](C)N(c2nc(=O)n3c4nc(c(F)cc24)-c2c(F)cccc2NC(=O)CCc2cccc(C(C)C)c2-3)C[C@H]1C ; C=CC(=O)N1C[C@H](C)N(c2nc(=O)n3c4nc(c(F)cc24)-c2c(F)cccc2NCCCc2cccc(C(C)C)c2-3)C[C@H]1C]

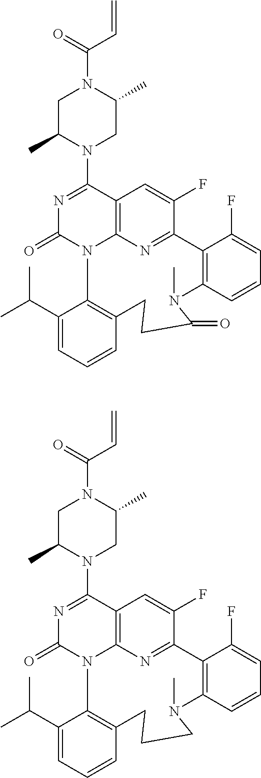 [SMILES: C=CC(=O)N1C[C@H](C)N(c2nc(=O)n3c4nc(c(F)cc24)-c2c(F)cccc2N(C)C(=O)CCc2cccc(C(C)C)c2-3)C[C@H]1C ; C=CC(=O)N1C[C@H](C)N(c2nc(=O)n3c4nc(c(F)cc24)-c2c(F)cccc2N(C)CCCc2cccc(C(C)C)c2-3)C[C@H]1C]

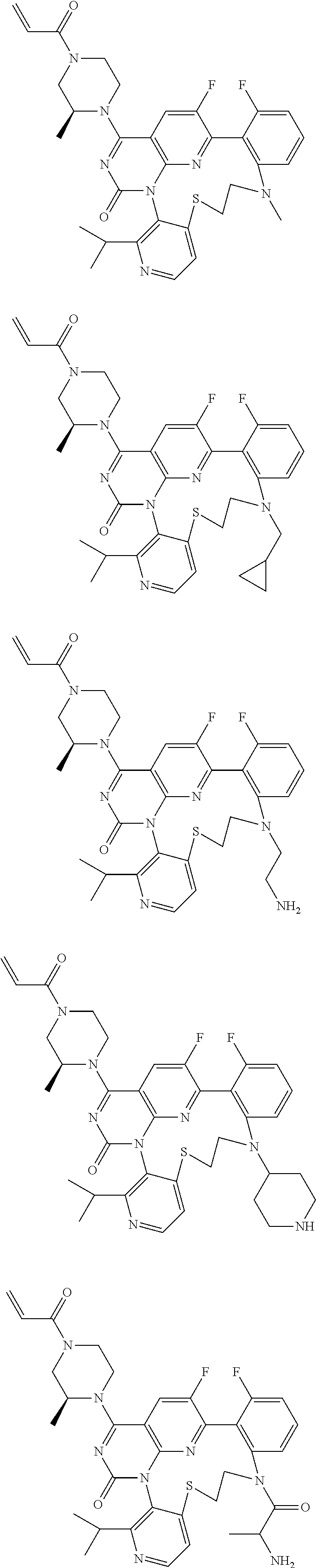 [SMILES: C=CC(=O)N1CCN(c2nc(=O)n3c4nc(c(F)cc24)-c2c(F)cccc2N(C)CCSc2ccnc(C(C)C)c2-3)[C@@H](C)C1 ; C=CC(=O)N1CCN(c2nc(=O)n3c4nc(c(F)cc24)-c2c(F)cccc2N(CC2CC2)CCSc2ccnc(C(C)C)c2-3)[C@@H](C)C1 ; C=CC(=O)N1CCN(c2nc(=O)n3c4nc(c(F)cc24)-c2c(F)cccc2N(CCN)CCSc2ccnc(C(C)C)c2-3)[C@@H](C)C1 ; C=CC(=O)N1CCN(c2nc(=O)n3c4nc(c(F)cc24)-c2c(F)cccc2N(C2CCNCC2)CCSc2ccnc(C(C)C)c2-3)[C@@H](C)C1 ; C=CC(=O)N1CCN(c2nc(=O)n3c4nc(c(F)cc24)-c2c(F)cccc2N(C(=O)C(C)N)CCSc2ccnc(C(C)C)c2-3)[C@@H](C)C1]

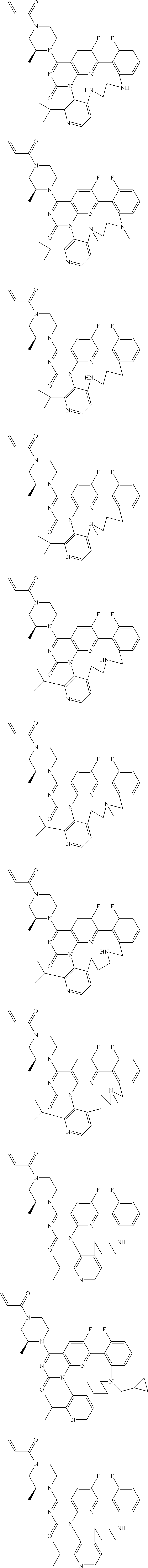

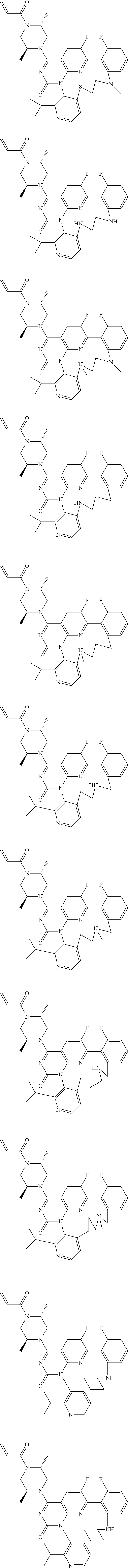

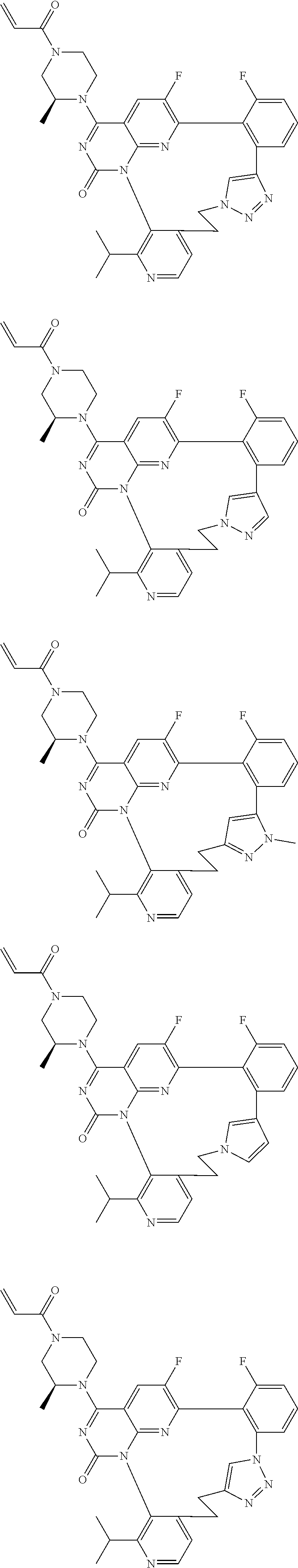 [SMILES: C=CC(=O)N1CCN(c2nc(=O)n3c4nc(c(F)cc24)-c2c(F)cccc2-c2cn(nn2)CCc2ccnc(C(C)C)c2-3)[C@@H](C)C1 ; C=CC(=O)N1CCN(c2nc(=O)n3c4nc(c(F)cc24)-c2c(F)cccc2-c2cnn(c2)CCc2ccnc(C(C)C)c2-3)[C@@H](C)C1 ; C=CC(=O)N1CCN(c2nc(=O)n3c4nc(c(F)cc24)-c2c(F)cccc2-c2cc(nn2C)CCc2ccnc(C(C)C)c2-3)[C@@H](C)C1 ; C=CC(=O)N1CCN(c2nc(=O)n3c4nc(c(F)cc24)-c2c(F)cccc2-c2ccn(c2)CCc2ccnc(C(C)C)c2-3)[C@@H](C)C1 ; C=CC(=O)N1CCN(c2nc(=O)n3c4nc(c(F)cc24)-c2c(F)cccc2-n2cc(nn2)CCc2ccnc(C(C)C)c2-3)[C@@H](C)C1]

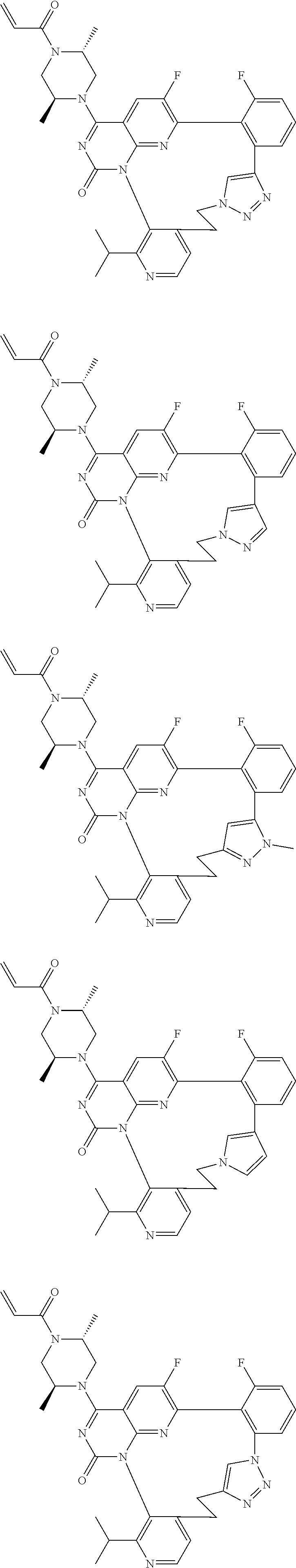 [SMILES: C=CC(=O)N1C[C@H](C)N(c2nc(=O)n3c4nc(c(F)cc24)-c2c(F)cccc2-c2cn(nn2)CCc2ccnc(C(C)C)c2-3)C[C@H]1C ; C=CC(=O)N1C[C@H](C)N(c2nc(=O)n3c4nc(c(F)cc24)-c2c(F)cccc2-c2cnn(c2)CCc2ccnc(C(C)C)c2-3)C[C@H]1C ; C=CC(=O)N1C[C@H](C)N(c2nc(=O)n3c4nc(c(F)cc24)-c2c(F)cccc2-c2cc(nn2C)CCc2ccnc(C(C)C)c2-3)C[C@H]1C ; C=CC(=O)N1C[C@H](C)N(c2nc(=O)n3c4nc(c(F)cc24)-c2c(F)cccc2-c2ccn(c2)CCc2ccnc(C(C)C)c2-3)C[C@H]1C ; C=CC(=O)N1C[C@H](C)N(c2nc(=O)n3c4nc(c(F)cc24)-c2c(F)cccc2-n2cc(nn2)CCc2ccnc(C(C)C)c2-3)C[C@H]1C]

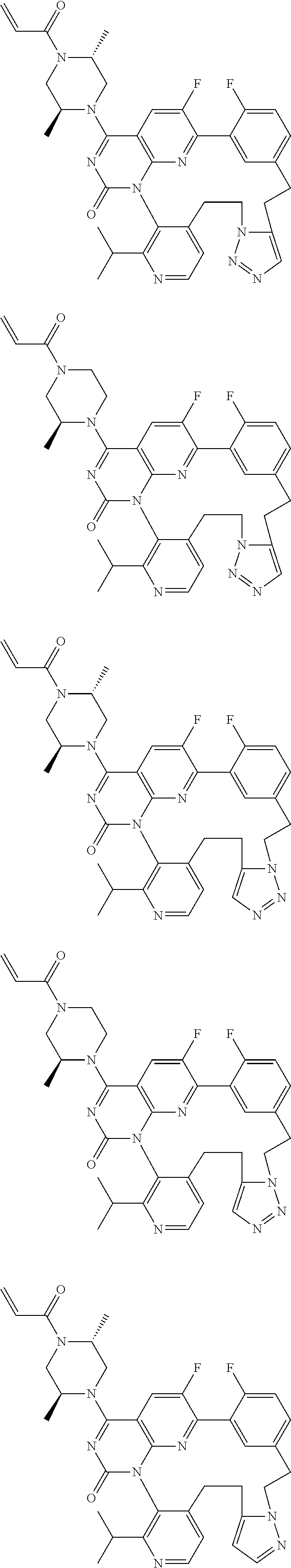 [SMILES: C=CC(=O)N1C[C@H](C)N(c2nc(=O)n3c4nc(c(F)cc24)-c2cc(ccc2F)CCc2cnnn2CCc2ccnc(C(C)C)c2-3)C[C@H]1C ; C=CC(=O)N1CCN(c2nc(=O)n3c4nc(c(F)cc24)-c2cc(ccc2F)CCc2cnnn2CCc2ccnc(C(C)C)c2-3)[C@@H](C)C1 ; C=CC(=O)N1C[C@H](C)N(c2nc(=O)n3c4nc(c(F)cc24)-c2cc(ccc2F)CCn2nncc2CCc2ccnc(C(C)C)c2-3)C[C@H]1C ; C=CC(=O)N1CCN(c2nc(=O)n3c4nc(c(F)cc24)-c2cc(ccc2F)CCn2nncc2CCc2ccnc(C(C)C)c2-3)[C@@H](C)C1 ; C=CC(=O)N1C[C@H](C)N(c2nc(=O)n3c4nc(c(F)cc24)-c2cc(ccc2F)CCn2nccc2CCc2ccnc(C(C)C)c2-3)C[C@H]1C]

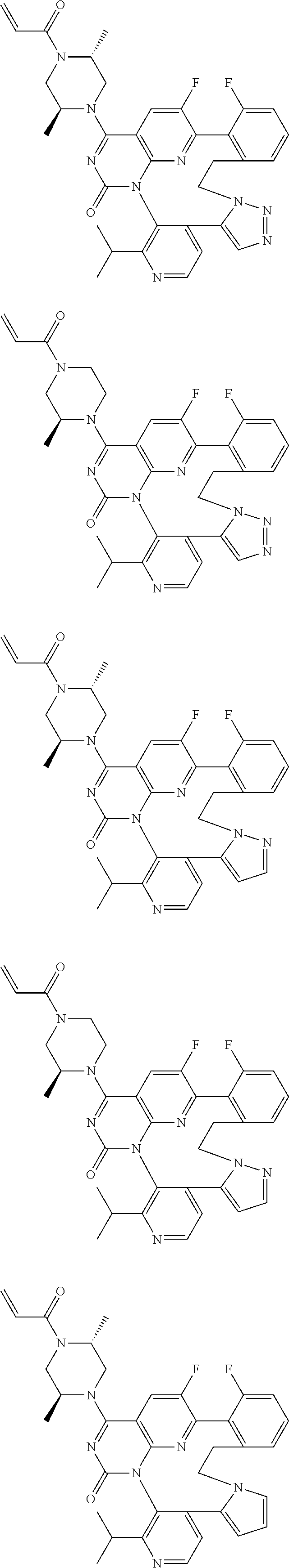 [SMILES: C=CC(=O)N1C[C@H](C)N(c2nc(=O)n3c4nc(c(F)cc24)-c2c(F)cccc2CCn2nncc2-c2ccnc(C(C)C)c2-3)C[C@H]1C ; C=CC(=O)N1CCN(c2nc(=O)n3c4nc(c(F)cc24)-c2c(F)cccc2CCn2nncc2-c2ccnc(C(C)C)c2-3)[C@@H](C)C1 ; C=CC(=O)N1C[C@H](C)N(c2nc(=O)n3c4nc(c(F)cc24)-c2c(F)cccc2CCn2nccc2-c2ccnc(C(C)C)c2-3)C[C@H]1C ; C=CC(=O)N1CCN(c2nc(=O)n3c4nc(c(F)cc24)-c2c(F)cccc2CCn2nccc2-c2ccnc(C(C)C)c2-3)[C@@H](C)C1 ; C=CC(=O)N1C[C@H](C)N(c2nc(=O)n3c4nc(c(F)cc24)-c2c(F)cccc2CCn2cccc2-c2ccnc(C(C)C)c2-3)C[C@H]1C]

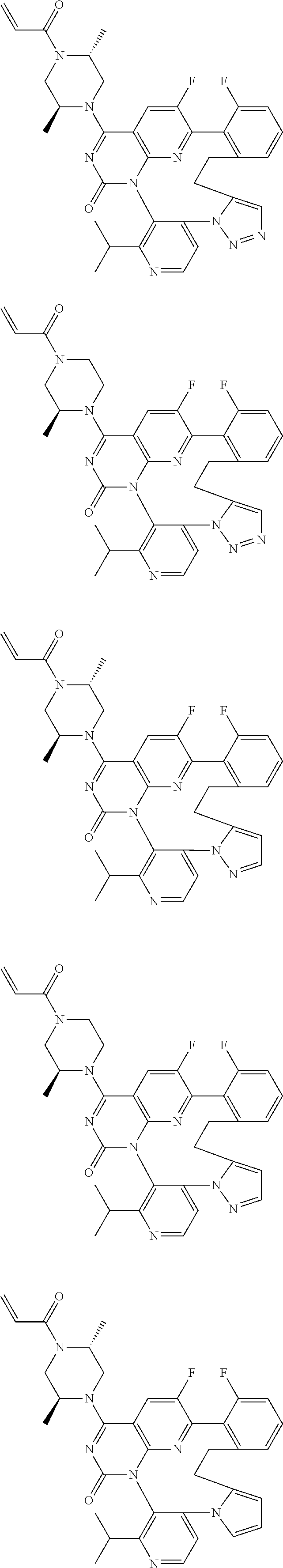 [SMILES: C=CC(=O)N1C[C@H](C)N(c2nc(=O)n3c4nc(c(F)cc24)-c2c(F)cccc2CCc2cnnn2-c2ccnc(C(C)C)c2-3)C[C@H]1C ; C=CC(=O)N1CCN(c2nc(=O)n3c4nc(c(F)cc24)-c2c(F)cccc2CCc2cnnn2-c2ccnc(C(C)C)c2-3)[C@@H](C)C1 ; C=CC(=O)N1C[C@H](C)N(c2nc(=O)n3c4nc(c(F)cc24)-c2c(F)cccc2CCc2ccnn2-c2ccnc(C(C)C)c2-3)C[C@H]1C ; C=CC(=O)N1CCN(c2nc(=O)n3c4nc(c(F)cc24)-c2c(F)cccc2CCc2ccnn2-c2ccnc(C(C)C)c2-3)[C@@H](C)C1 ; C=CC(=O)N1C[C@H](C)N(c2nc(=O)n3c4nc(c(F)cc24)-c2c(F)cccc2CCc2cccn2-c2ccnc(C(C)C)c2-3)C[C@H]1C]

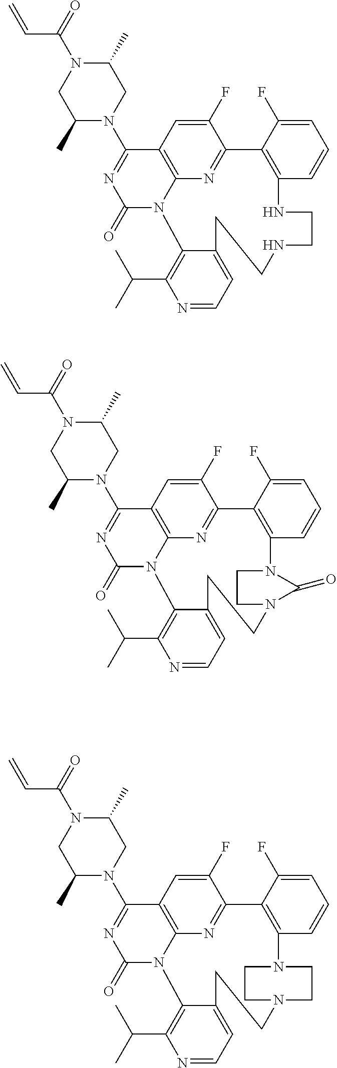 [SMILES: C=CC(=O)N1C[C@H](C)N(c2nc(=O)n3c4nc(c(F)cc24)-c2c(F)cccc2NCCNCCc2ccnc(C(C)C)c2-3)C[C@H]1C ; C=CC(=O)N1C[C@H](C)N(c2nc(=O)n3c4nc(c(F)cc24)-c2c(F)cccc2N2CCN(CCc4ccnc(C(C)C)c4-3)C2=O)C[C@H]1C ; C=CC(=O)N1C[C@H](C)N(c2nc(=O)n3c4nc(c(F)cc24)-c2c(F)cccc2N2CCN(CCc4ccnc(C(C)C)c4-3)CC2)C[C@H]1C]

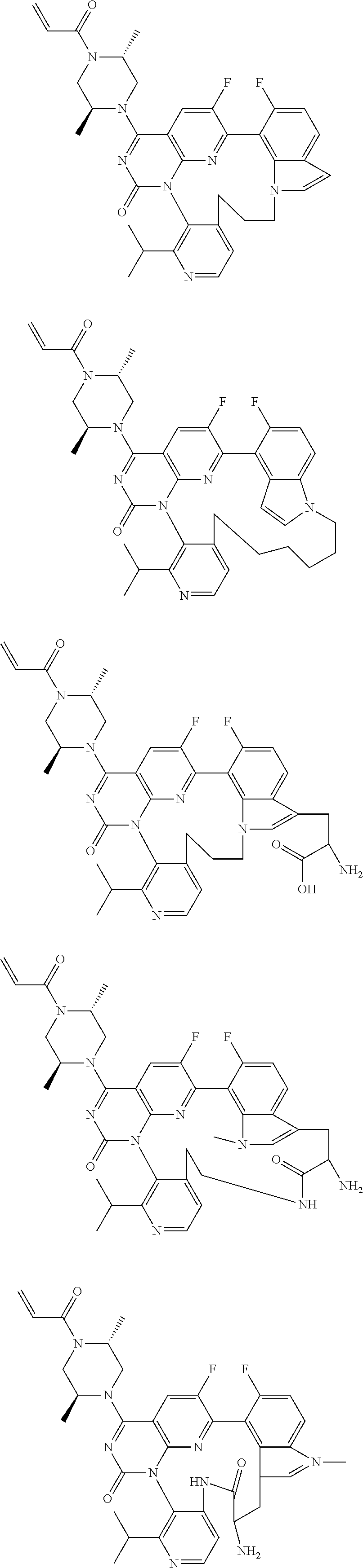

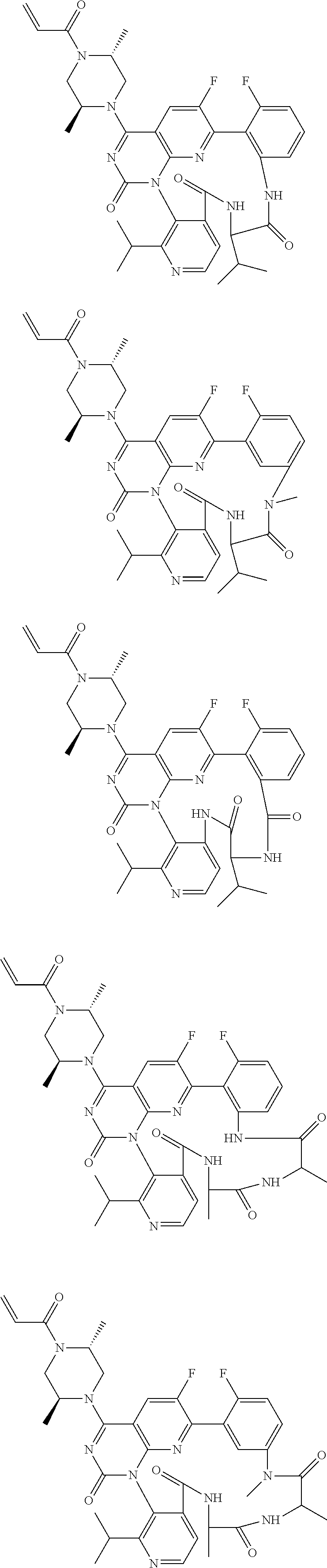

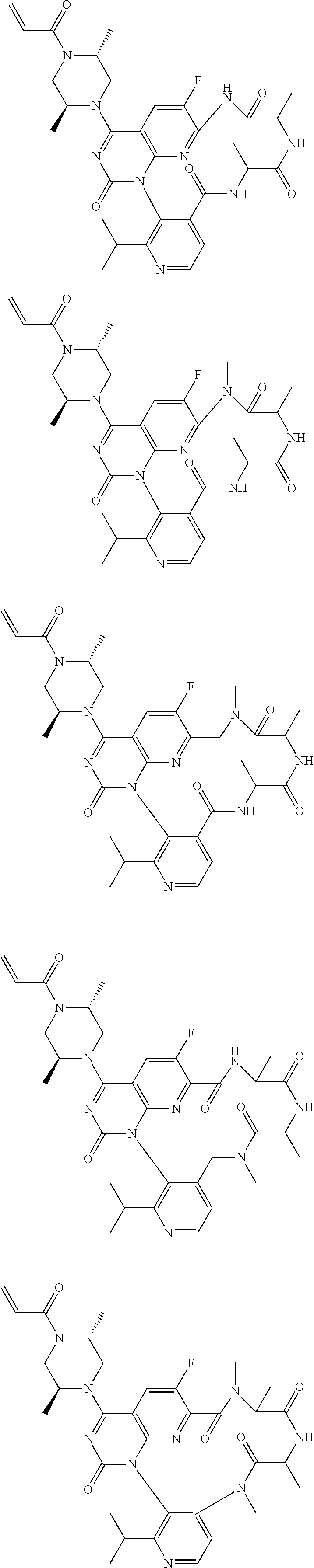

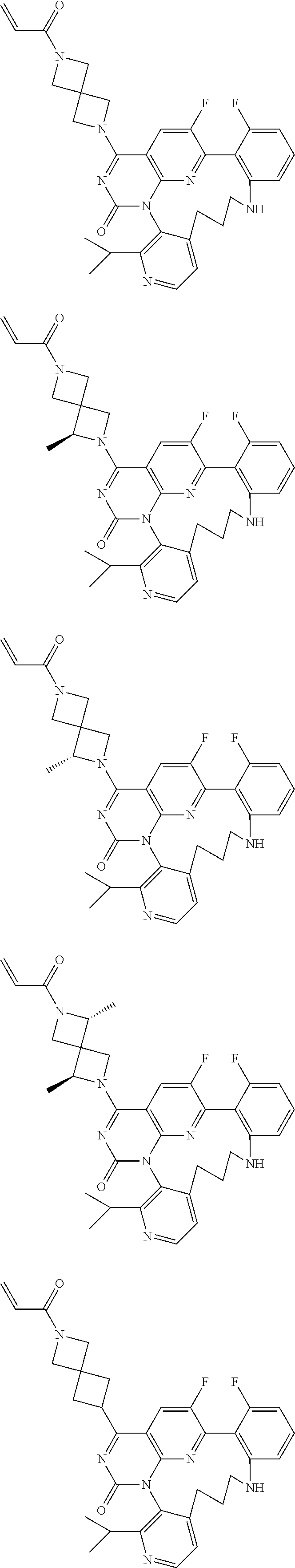 [SMILES: C=CC(=O)N1CC2(C1)CN(c1nc(=O)n3c4nc(c(F)cc14)-c1c(F)cccc1NCCCc1ccnc(C(C)C)c1-3)C2 ; C=CC(=O)N1CC2(C1)CN(c1nc(=O)n3c4nc(c(F)cc14)-c1c(F)cccc1NCCCc1ccnc(C(C)C)c1-3)[C@H]2C ; C=CC(=O)N1CC2(C1)CN(c1nc(=O)n3c4nc(c(F)cc14)-c1c(F)cccc1NCCCc1ccnc(C(C)C)c1-3)[C@@H]2C ; C=CC(=O)N1CC2(CN(c3nc(=O)n4c5nc(c(F)cc35)-c3c(F)cccc3NCCCc3ccnc(C(C)C)c3-4)[C@H]2C)[C@H]1C ; C=CC(=O)N1CC2(CC(c3nc(=O)n4c5nc(c(F)cc35)-c3c(F)cccc3NCCCc3ccnc(C(C)C)c3-4)C2)C1]

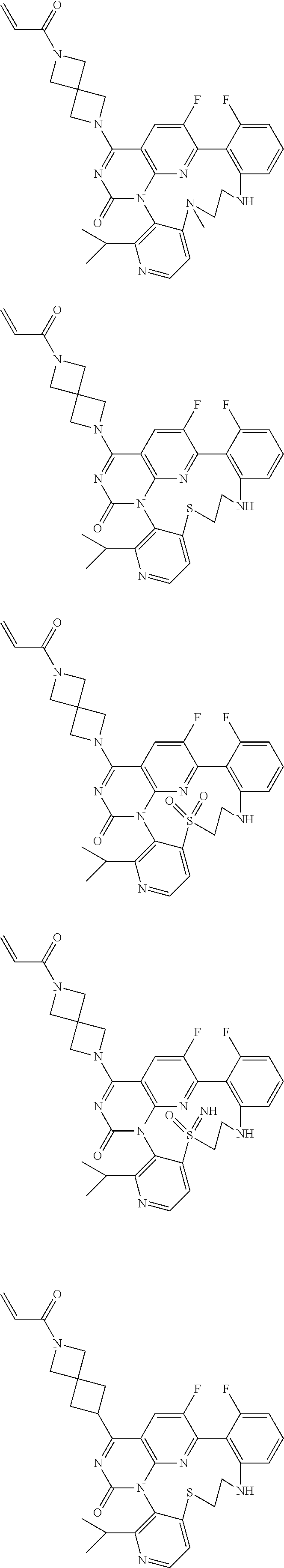 [SMILES: C=CC(=O)N1CC2(C1)CN(c1nc(=O)n3c4nc(c(F)cc14)-c1c(F)cccc1NCCN(C)c1ccnc(C(C)C)c1-3)C2 ; C=CC(=O)N1CC2(C1)CN(c1nc(=O)n3c4nc(c(F)cc14)-c1c(F)cccc1NCCSc1ccnc(C(C)C)c1-3)C2 ; C=CC(=O)N1CC2(C1)CN(c1nc(=O)n3c4nc(c(F)cc14)-c1c(F)cccc1NCCS(=O)(=O)c1ccnc(C(C)C)c1-3)C2 ; C=CC(=O)N1CC2(C1)CN(c1nc(=O)n3c4nc(c(F)cc14)-c1c(F)cccc1NCCS(=N)(=O)c1ccnc(C(C)C)c1-3)C2 ; C=CC(=O)N1CC2(CC(c3nc(=O)n4c5nc(c(F)cc35)-c3c(F)cccc3NCCSc3ccnc(C(C)C)c3-4)C2)C1]

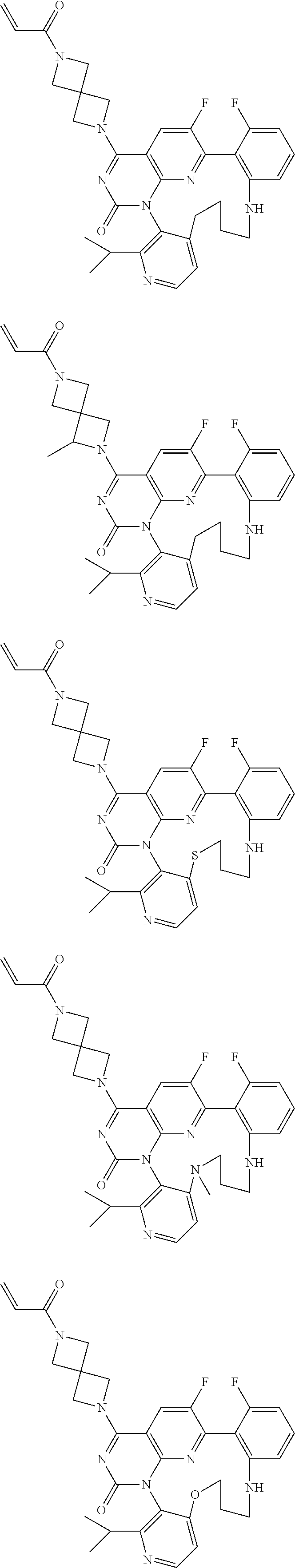 [SMILES: C=CC(=O)N1CC2(C1)CN(c1nc(=O)n3c4nc(c(F)cc14)-c1c(F)cccc1NCCCCc1ccnc(C(C)C)c1-3)C2 ; C=CC(=O)N1CC2(C1)CN(c1nc(=O)n3c4nc(c(F)cc14)-c1c(F)cccc1NCCCCc1ccnc(C(C)C)c1-3)C2C ; C=CC(=O)N1CC2(C1)CN(c1nc(=O)n3c4nc(c(F)cc14)-c1c(F)cccc1NCCCSc1ccnc(C(C)C)c1-3)C2 ; C=CC(=O)N1CC2(C1)CN(c1nc(=O)n3c4nc(c(F)cc14)-c1c(F)cccc1NCCCN(C)c1ccnc(C(C)C)c1-3)C2 ; C=CC(=O)N1CC2(C1)CN(c1nc(=O)n3c4nc(c(F)cc14)-c1c(F)cccc1NCCCOc1ccnc(C(C)C)c1-3)C2]

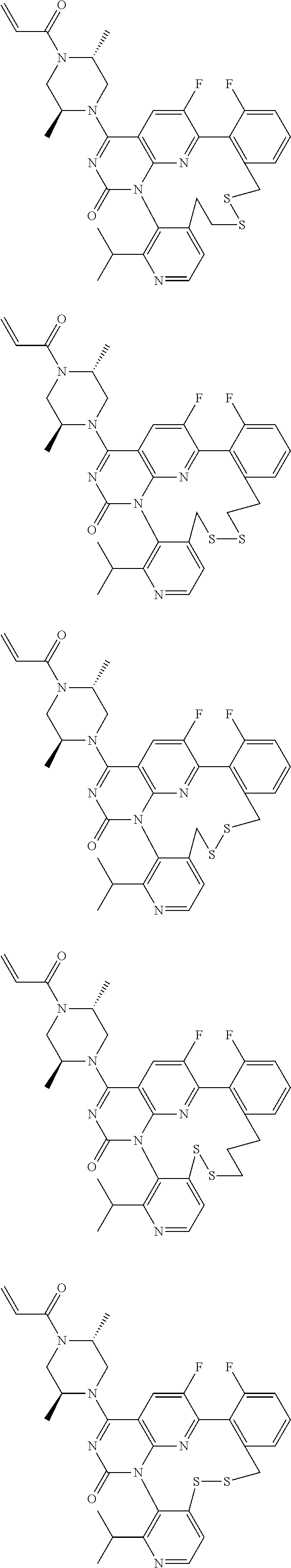 [SMILES: C=CC(=O)N1C[C@H](C)N(c2nc(=O)n3c4nc(c(F)cc24)-c2c(F)cccc2CSSCCc2ccnc(C(C)C)c2-3)C[C@H]1C ; C=CC(=O)N1C[C@H](C)N(c2nc(=O)n3c4nc(c(F)cc24)-c2c(F)cccc2CCSSCc2ccnc(C(C)C)c2-3)C[C@H]1C ; C=CC(=O)N1C[C@H](C)N(c2nc(=O)n3c4nc(c(F)cc24)-c2c(F)cccc2CSSCc2ccnc(C(C)C)c2-3)C[C@H]1C ; C=CC(=O)N1C[C@H](C)N(c2nc(=O)n3c4nc(c(F)cc24)-c2c(F)cccc2CCCSSc2ccnc(C(C)C)c2-3)C[C@H]1C ; C=CC(=O)N1C[C@H](C)N(c2nc(=O)n3c4nc(c(F)cc24)-c2c(F)cccc2CSSc2ccnc(C(C)C)c2-3)C[C@H]1C]

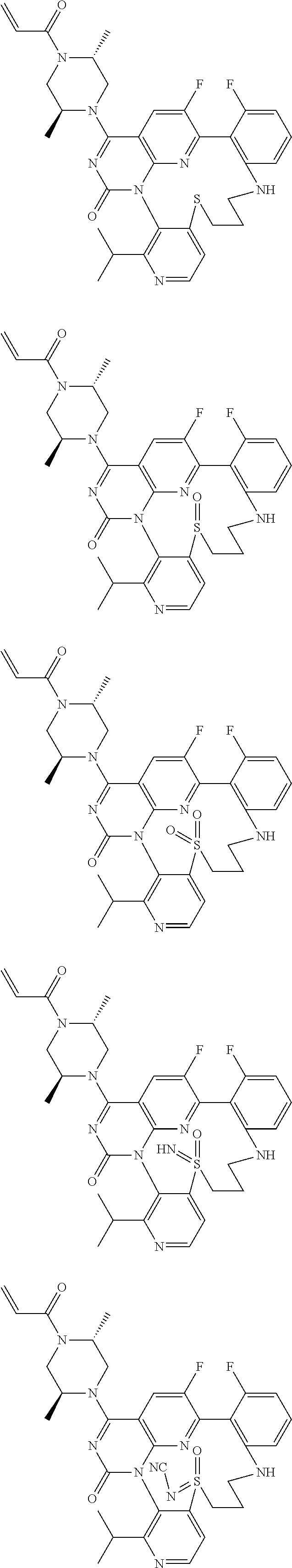 [SMILES: C=CC(=O)N1C[C@H](C)N(c2nc(=O)n3c4nc(c(F)cc24)-c2c(F)cccc2NCCCSc2ccnc(C(C)C)c2-3)C[C@H]1C ; C=CC(=O)N1C[C@H](C)N(c2nc(=O)n3c4nc(c(F)cc24)-c2c(F)cccc2NCCCS(=O)c2ccnc(C(C)C)c2-3)C[C@H]1C ; C=CC(=O)N1C[C@H](C)N(c2nc(=O)n3c4nc(c(F)cc24)-c2c(F)cccc2NCCCS(=O)(=O)c2ccnc(C(C)C)c2-3)C[C@H]1C ; C=CC(=O)N1C[C@H](C)N(c2nc(=O)n3c4nc(c(F)cc24)-c2c(F)cccc2NCCCS(=N)(=O)c2ccnc(C(C)C)c2-3)C[C@H]1C ; C=CC(=O)N1C[C@H](C)N(c2nc(=O)n3c4nc(c(F)cc24)-c2c(F)cccc2NCCCS(=O)(=NC#N)c2ccnc(C(C)C)c2-3)C[C@H]1C]

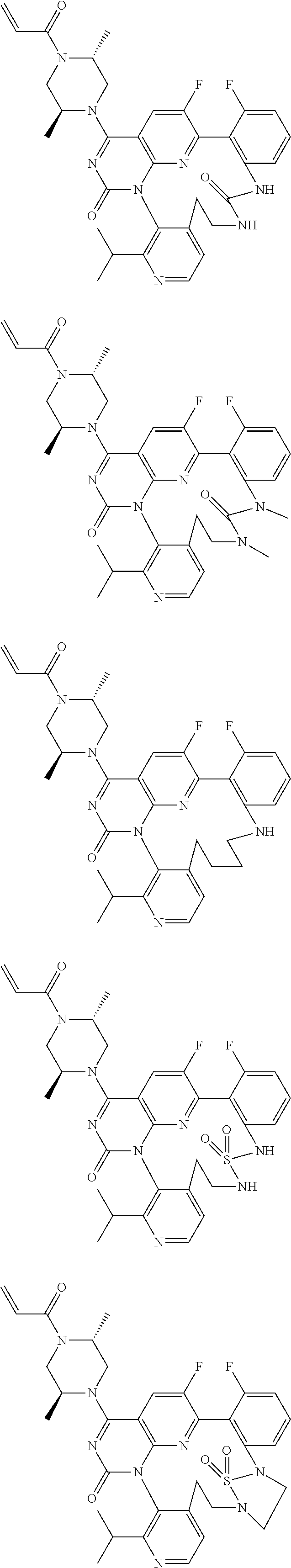 [SMILES: C=CC(=O)N1C[C@H](C)N(c2nc(=O)n3c4nc(c(F)cc24)-c2c(F)cccc2NC(=O)NCCc2ccnc(C(C)C)c2-3)C[C@H]1C ; C=CC(=O)N1C[C@H](C)N(c2nc(=O)n3c4nc(c(F)cc24)-c2c(F)cccc2N(C)C(=O)N(C)CCc2ccnc(C(C)C)c2-3)C[C@H]1C ; C=CC(=O)N1C[C@H](C)N(c2nc(=O)n3c4nc(c(F)cc24)-c2c(F)cccc2NCCCCc2ccnc(C(C)C)c2-3)C[C@H]1C ; C=CC(=O)N1C[C@H](C)N(c2nc(=O)n3c4nc(c(F)cc24)-c2c(F)cccc2NS(=O)(=O)NCCc2ccnc(C(C)C)c2-3)C[C@H]1C ; C=CC(=O)N1C[C@H](C)N(c2nc(=O)n3c4nc(c(F)cc24)-c2c(F)cccc2N2CCN(CCc4ccnc(C(C)C)c4-3)S2(=O)=O)C[C@H]1C]

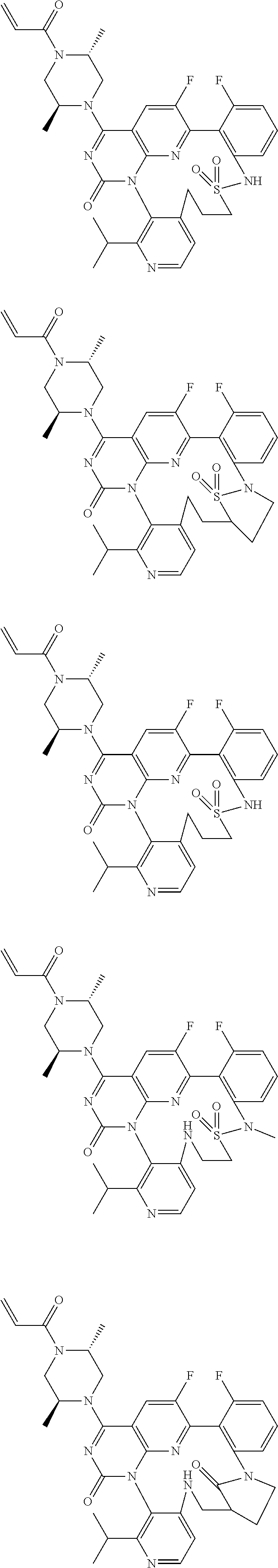 [SMILES: C=CC(=O)N1C[C@H](C)N(c2nc(=O)n3c4nc(c(F)cc24)-c2c(F)cccc2NS(=O)(=O)CCCc2ccnc(C(C)C)c2-3)C[C@H]1C ; C=CC(=O)N1C[C@H](C)N(c2nc(=O)n3c4nc(c(F)cc24)-c2c(F)cccc2N2CCC(CCc4ccnc(C(C)C)c4-3)S2(=O)=O)C[C@H]1C ; C=CC(=O)N1C[C@H](C)N(c2nc(=O)n3c4nc(c(F)cc24)-c2c(F)cccc2NS(=O)(=O)CCCc2ccnc(C(C)C)c2-3)C[C@H]1C ; C=CC(=O)N1C[C@H](C)N(c2nc(=O)n3c4nc(c(F)cc24)-c2c(F)cccc2N(C)S(=O)(=O)CCNc2ccnc(C(C)C)c2-3)C[C@H]1C ; C=CC(=O)N1C[C@H](C)N(c2nc(=O)n3c4nc(c(F)cc24)-c2c(F)cccc2N2CCC(CNc4ccnc(C(C)C)c4-3)C2=O)C[C@H]1C]

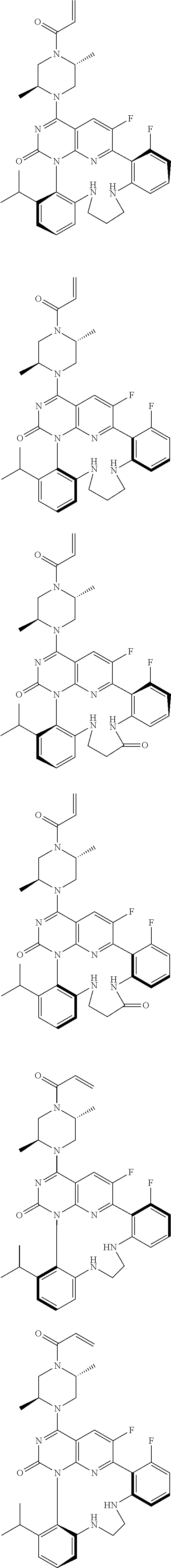

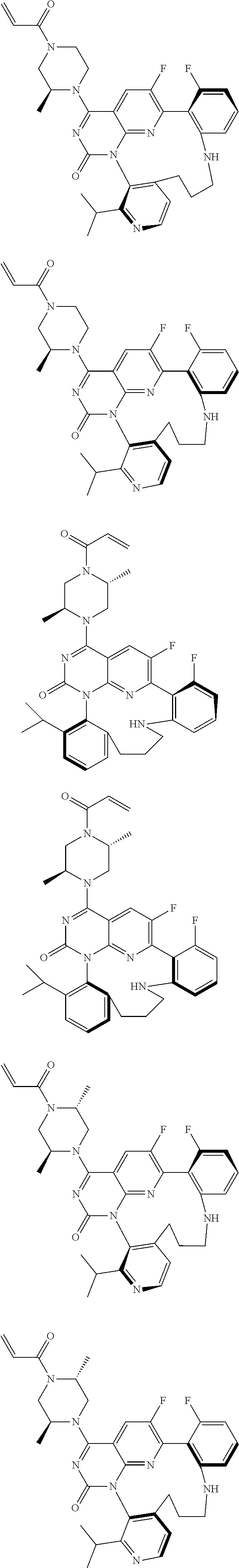

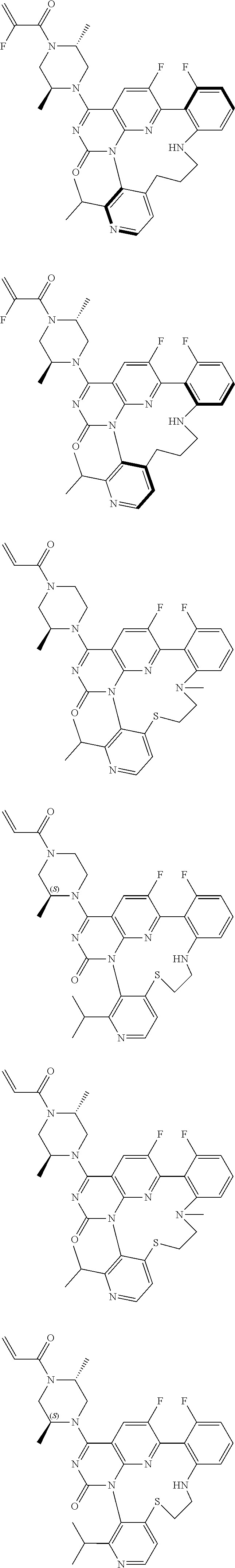

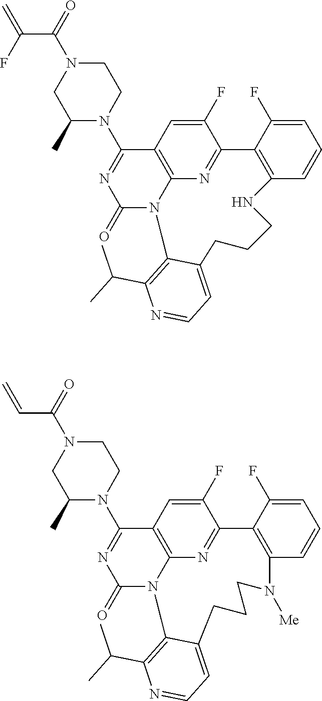 [SMILES: C=C(F)C(=O)N1CCN(c2nc(=O)n3c4nc(c(F)cc24)-c2c(F)cccc2NCCCc2ccnc(C(C)C)c2-3)[C@@H](C)C1 ; C=CC(=O)N1CCN(c2nc(=O)n3c4nc(c(F)cc24)-c2c(F)cccc2N(C)CCCc2ccnc(C(C)C)c2-3)[C@@H](C)C1]

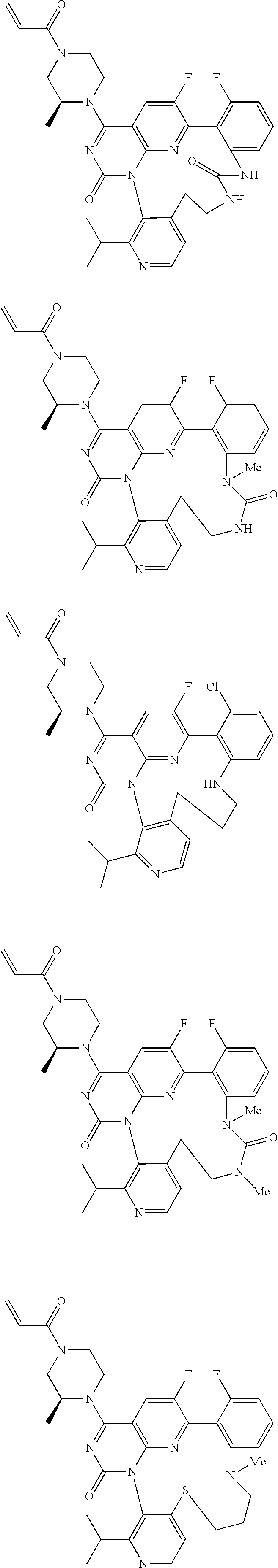 [SMILES: C=CC(=O)N1CCN(c2nc(=O)n3c4nc(c(F)cc24)-c2c(F)cccc2NC(=O)NCCc2ccnc(C(C)C)c2-3)[C@@H](C)C1 ; C=CC(=O)N1CCN(c2nc(=O)n3c4nc(c(F)cc24)-c2c(F)cccc2N(C)C(=O)NCCc2ccnc(C(C)C)c2-3)[C@@H](C)C1 ; C=CC(=O)N1CCN(c2nc(=O)n3c4nc(c(F)cc24)-c2c(Cl)cccc2NCCCc2ccnc(C(C)C)c2-3)[C@@H](C)C1 ; C=CC(=O)N1CCN(c2nc(=O)n3c4nc(c(F)cc24)-c2c(F)cccc2N(C)C(=O)N(C)CCc2ccnc(C(C)C)c2-3)[C@@H](C)C1 ; C=CC(=O)N1CCN(c2nc(=O)n3c4nc(c(F)cc24)-c2c(F)cccc2N(C)CCCSc2ccnc(C(C)C)c2-3)[C@@H](C)C1]

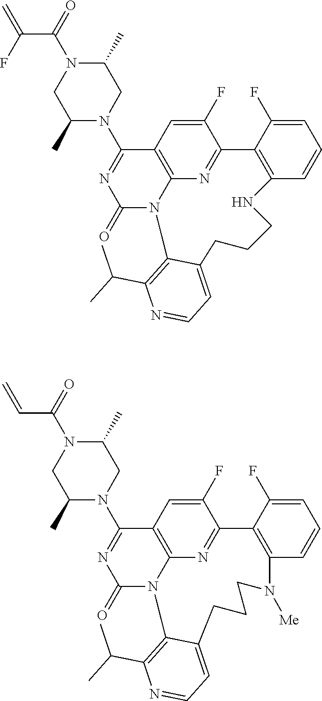 [SMILES: C=C(F)C(=O)N1C[C@H](C)N(c2nc(=O)n3c4nc(c(F)cc24)-c2c(F)cccc2NCCCc2ccnc(C(C)C)c2-3)C[C@H]1C ; C=CC(=O)N1C[C@H](C)N(c2nc(=O)n3c4nc(c(F)cc24)-c2c(F)cccc2N(C)CCCc2ccnc(C(C)C)c2-3)C[C@H]1C]

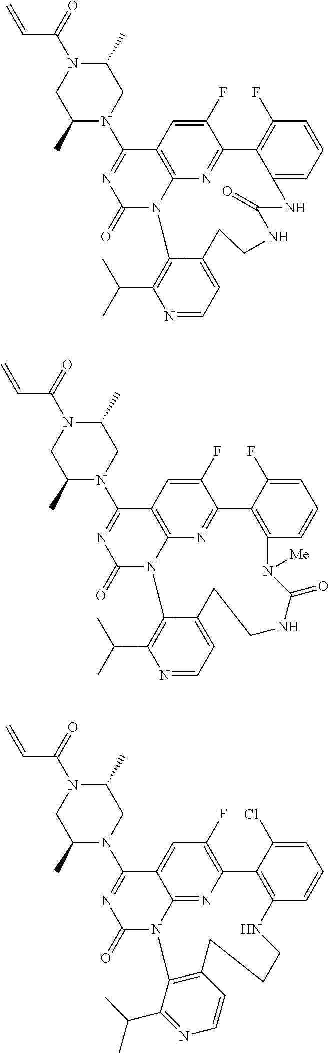 [SMILES: C=CC(=O)N1C[C@H](C)N(c2nc(=O)n3c4nc(c(F)cc24)-c2c(F)cccc2NC(=O)NCCc2ccnc(C(C)C)c2-3)C[C@H]1C ; C=CC(=O)N1C[C@H](C)N(c2nc(=O)n3c4nc(c(F)cc24)-c2c(F)cccc2N(C)C(=O)NCCc2ccnc(C(C)C)c2-3)C[C@H]1C ; C=CC(=O)N1C[C@H](C)N(c2nc(=O)n3c4nc(c(F)cc24)-c2c(Cl)cccc2NCCCc2ccnc(C(C)C)c2-3)C[C@H]1C]

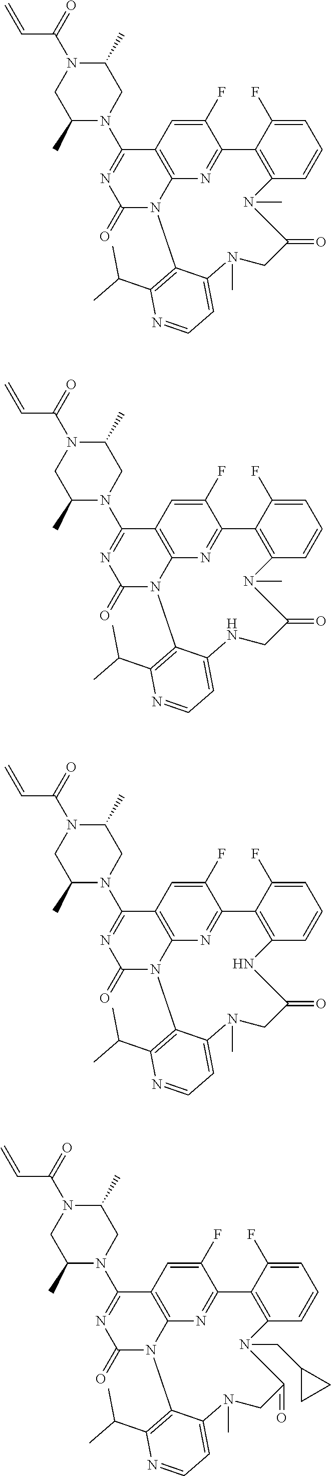 [SMILES: C=CC(=O)N1C[C@H](C)N(c2nc(=O)n3c4nc(c(F)cc24)-c2c(F)cccc2N(C)C(=O)CN(C)c2ccnc(C(C)C)c2-3)C[C@H]1C ; C=CC(=O)N1C[C@H](C)N(c2nc(=O)n3c4nc(c(F)cc24)-c2c(F)cccc2N(C)C(=O)CNc2ccnc(C(C)C)c2-3)C[C@H]1C ; C=CC(=O)N1C[C@H](C)N(c2nc(=O)n3c4nc(c(F)cc24)-c2c(F)cccc2NC(=O)CN(C)c2ccnc(C(C)C)c2-3)C[C@H]1C ; C=CC(=O)N1C[C@H](C)N(c2nc(=O)n3c4nc(c(F)cc24)-c2c(F)cccc2N(CC2CC2)C(=O)CN(C)c2ccnc(C(C)C)c2-3)C[C@H]1C]

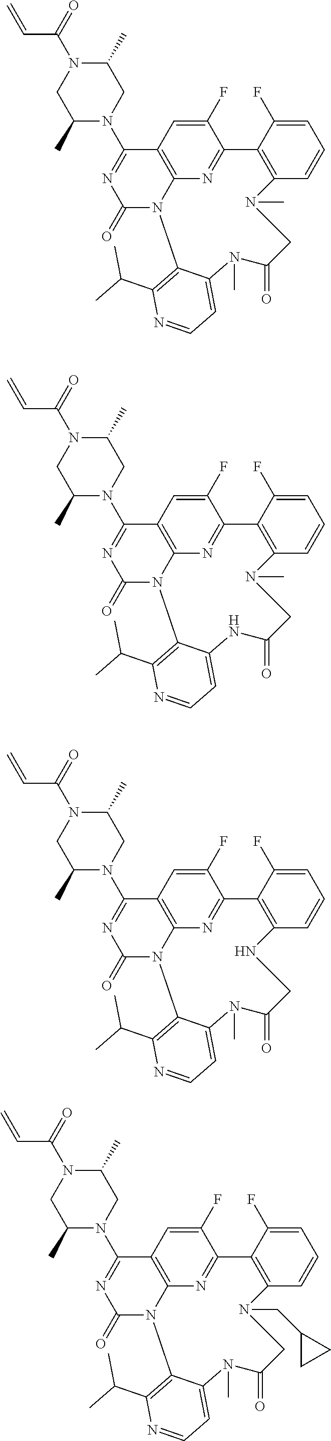 [SMILES: C=CC(=O)N1C[C@H](C)N(c2nc(=O)n3c4nc(c(F)cc24)-c2c(F)cccc2N(C)CC(=O)N(C)c2ccnc(C(C)C)c2-3)C[C@H]1C ; C=CC(=O)N1C[C@H](C)N(c2nc(=O)n3c4nc(c(F)cc24)-c2c(F)cccc2N(C)CC(=O)Nc2ccnc(C(C)C)c2-3)C[C@H]1C ; C=CC(=O)N1C[C@H](C)N(c2nc(=O)n3c4nc(c(F)cc24)-c2c(F)cccc2NCC(=O)N(C)c2ccnc(C(C)C)c2-3)C[C@H]1C ; C=CC(=O)N1C[C@H](C)N(c2nc(=O)n3c4nc(c(F)cc24)-c2c(F)cccc2N(CC2CC2)CC(=O)N(C)c2ccnc(C(C)C)c2-3)C[C@H]1C]

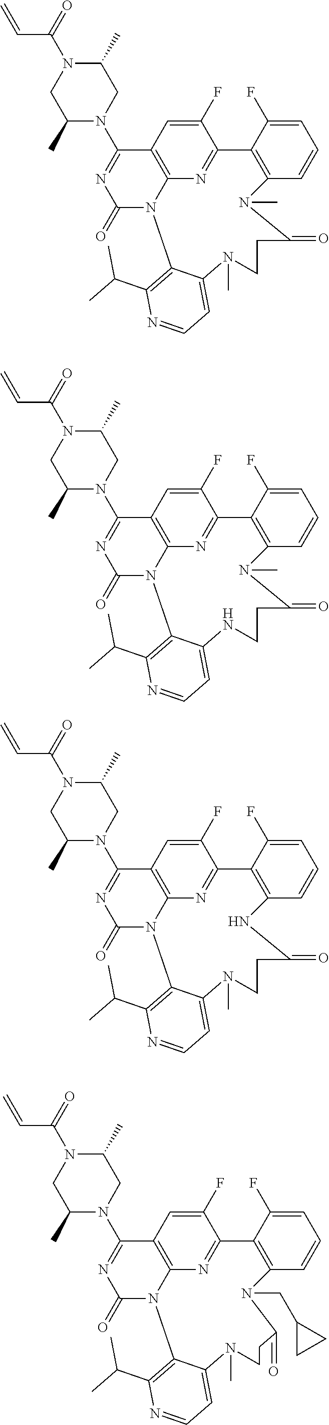 [SMILES: C=CC(=O)N1C[C@H](C)N(c2nc(=O)n3c4nc(c(F)cc24)-c2c(F)cccc2N(C)C(=O)CCN(C)c2ccnc(C(C)C)c2-3)C[C@H]1C ; C=CC(=O)N1C[C@H](C)N(c2nc(=O)n3c4nc(c(F)cc24)-c2c(F)cccc2N(C)C(=O)CCNc2ccnc(C(C)C)c2-3)C[C@H]1C ; C=CC(=O)N1C[C@H](C)N(c2nc(=O)n3c4nc(c(F)cc24)-c2c(F)cccc2NC(=O)CCN(C)c2ccnc(C(C)C)c2-3)C[C@H]1C ; C=CC(=O)N1C[C@H](C)N(c2nc(=O)n3c4nc(c(F)cc24)-c2c(F)cccc2N(CC2CC2)C(=O)CCN(C)c2ccnc(C(C)C)c2-3)C[C@H]1C]

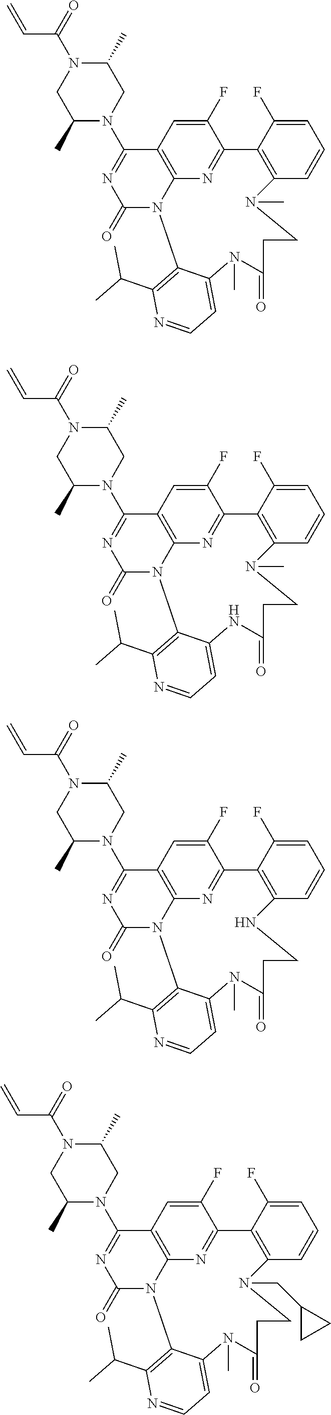 [SMILES: C=CC(=O)N1C[C@H](C)N(c2nc(=O)n3c4nc(c(F)cc24)-c2c(F)cccc2N(C)CCC(=O)N(C)c2ccnc(C(C)C)c2-3)C[C@H]1C ; C=CC(=O)N1C[C@H](C)N(c2nc(=O)n3c4nc(c(F)cc24)-c2c(F)cccc2N(C)CCC(=O)Nc2ccnc(C(C)C)c2-3)C[C@H]1C ; C=CC(=O)N1C[C@H](C)N(c2nc(=O)n3c4nc(c(F)cc24)-c2c(F)cccc2NCCC(=O)N(C)c2ccnc(C(C)C)c2-3)C[C@H]1C ; C=CC(=O)N1C[C@H](C)N(c2nc(=O)n3c4nc(c(F)cc24)-c2c(F)cccc2N(CC2CC2)CCC(=O)N(C)c2ccnc(C(C)C)c2-3)C[C@H]1C]

4. A method of treating cancer in a subject comprising administering to the subject a therapeutically effective amount of the compound of claim 1 or a pharmaceutically acceptable salt thereof alone or together with a therapeutically effective amount of any other anticancer drug.
